(19) United States
(12) Patent Application Publication
(10) Pub. No.: US 2024/0269308 A1
(43) Pub. Date: Aug. 15, 2024

(54) PRODRUGS OF TOPOISOMERASE I INHIBITOR FOR ADC CONJUGATIONS AND METHODS OF USE THEREOF

(71) Applicant: Regeneron Pharmaceuticals, Inc., Tarrytown, NY (US)

(72) Inventor: Amy HAN, Hockessin, DE (US)

(73) Assignee: Regeneron Pharmaceuticals, Inc., Tarrytown, NY (US)

(21) Appl. No.: 18/393,062

(22) Filed: Dec. 21, 2023

Related U.S. Application Data
(60) Provisional application No. 63/434,230, filed on Dec. 21, 2022.
(60) Provisional application No. 63/472,064, filed on Jun. 9, 2023.

(51) Int. Cl. A61K 47/68; A61P 35/00

(52) U.S. Cl. CPC A61K 47/68037; A61K 47/6889; A61P 35/00

(57) ABSTRACT

Described herein are protein-drug conjugates and compositions thereof that are useful, for example, for target-specific delivery of therapeutic moieties, e.g., camptothecin analogs and/or derivatives. In certain embodiments, provided are specific and efficient methods for producing protein-drug constructs (e.g., antibody-drug conjugates) utilizing a combination of transglutaminase and 1,3-cycloaddition techniques. Camptothecin analogs, antibody-drug conjugates, and compositions which comprise glutaminyl-modified antibodies and camptothecin analog payloads and are provided.

### CROSS REFERENCE TO RELATED APPLICATION

This patent application claims the benefit of U.S. Provisional Application No. 63/472,064, filed on Jun. 9, 2023, and 63/434,230, filed on Dec. 21, 2022, the disclosure of each of which is incorporated by reference herein in its entirety.

### FIELD OF THE DISCLOSURE

The present disclosure relates to protein-drug conjugates (e.g., antibody-drug conjugates), pharmaceutical compositions, and methods of treating disease therewith. Also provided are specific and efficient methods for producing protein-drug constructs utilizing a combination of transglutaminase and 1,3-cycloaddition techniques. More specifically, the present disclosure relates to prodrugs of topoisomerase I inhibitor for ADC conjugations and methods of use thereof.

### SEQUENCE LISTING

The instant application contains a Sequence Listing which has been submitted electronically in XML file format and is hereby incorporated by reference in its entirety. Said XML copy, created on Dec. 21, 2023, is named 250298_000607_SL.xml and is 2,619,378 bytes in size.

### BACKGROUND OF THE DISCLOSURE

Proliferative diseases are characterized by uncontrolled growth and spread of abnormal cells. If the spread is not controlled, it can result in death. Abnormal proliferation, for example, cancer, is caused by both external factors (e.g., tobacco, chemicals, radiation and infectious organisms) and internal factors (inherited mutations, immune system conditions, the mutations that occur from metabolism). These causal factors may act together or in sequence to initiate or promote abnormal proliferation. Cancer is treated by surgery, radiation, chemotherapy, hormones and immunotherapy. However, there is a need for more effective anti-proliferation drugs.

The ideal anti-proliferation therapy would enable targeted delivery of highly cytotoxic agents to tumor cells and would leave normal cells unaffected. Conventional chemotherapeutic treatment is limited because of the toxic side-effects that arise from effects of the drug on non-cancerous cells. Various approaches to targeted drug delivery have been tried, including the use of conjugates of tumor targeted probes (such as antibodies or growth factors) with toxins such as pseudomonas or diphtheria toxins, which arrest the synthesis of proteins and cells. However, the side effects include reaction of the immune system due to non-human components of the conjugates. Further, the half-life of the drug conjugates was limited due to elimination from the circulation through renal filtration, and schematic degradation, uptake by the reticuloendothelial system (RES), and accumulation in non-targeted organs and tissues.

Another approach uses passive drug carriers such as polymers, liposomes, and polymeric micelles to take advantage of the hyper-permeability of vascular endothelia of tumor tissue. Polymeric drugs and macromolecules accumulate within solid tumors due to an enhanced permeability and retention mechanism. However, barriers of using such targeted deliveries include fast clearance of foreign particles from the blood, and technological hindrances in obtaining highly standardized, pharmaceutically acceptable drug delivery systems with the necessary specificity and selectivity for binding tumor cells.

Protein conjugates, such as antibody conjugates, utilize the selective binding of a binding agent to deliver a payload to targets within tissues of subjects. The payload can be a therapeutic moiety that is capable of taking action at the target.

Several techniques for conjugating linkers and payloads to antibodies are available. Many conjugates are prepared by non-selective covalent linkage to cysteine or lysine residues in the antibody. This non-selective technique can result in a heterogeneous mixture of products with conjugations at different sites and with different numbers of conjugations per antibody. Thus, there is a need in the art for methods and techniques that provide site-selective antibody conjugation.

There is a need in the art for additional safe and effective anti-tumor targeting agents that can bind to various antigens to provide enhanced the treatment of diseases such as cancer for use in monotherapy and combination therapies. In certain embodiments, the present disclosure meets the needs and provides other advantages.

The foregoing discussion is presented solely to provide a better understanding of the nature of the problems confronting the art and should not be construed in any way as an admission as to prior art nor should the citation of any reference herein be construed as an admission that such reference constitutes “prior art” to the instant application.

### SUMMARY OF THE DISCLOSURE

Various non-limiting aspects and embodiments of the disclosure are described below.

In one aspect, the present disclosure provides an antibody-drug conjugate comprising an antibody or an antigen-binding fragment thereof conjugated to a compound having Formula (I)

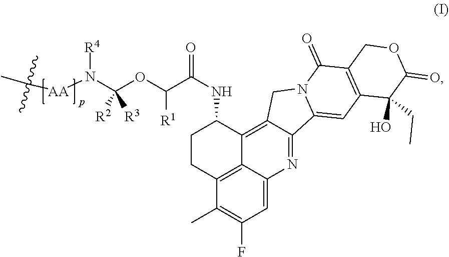

or a pharmaceutically acceptable salt thereof,
wherein R¹, R², R³, and R⁴ are independently hydrogen, a C₁₋₅ alkyl, or aryl;
AA is a natural or a nonnatural amino acid;
p is an integer from 1 to 6, and

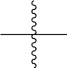

indicates the point of attachment to the antibody or the antigen-binding fragment thereof, directly or via a linker.

In one embodiment, said compound of Formula (I) comprises

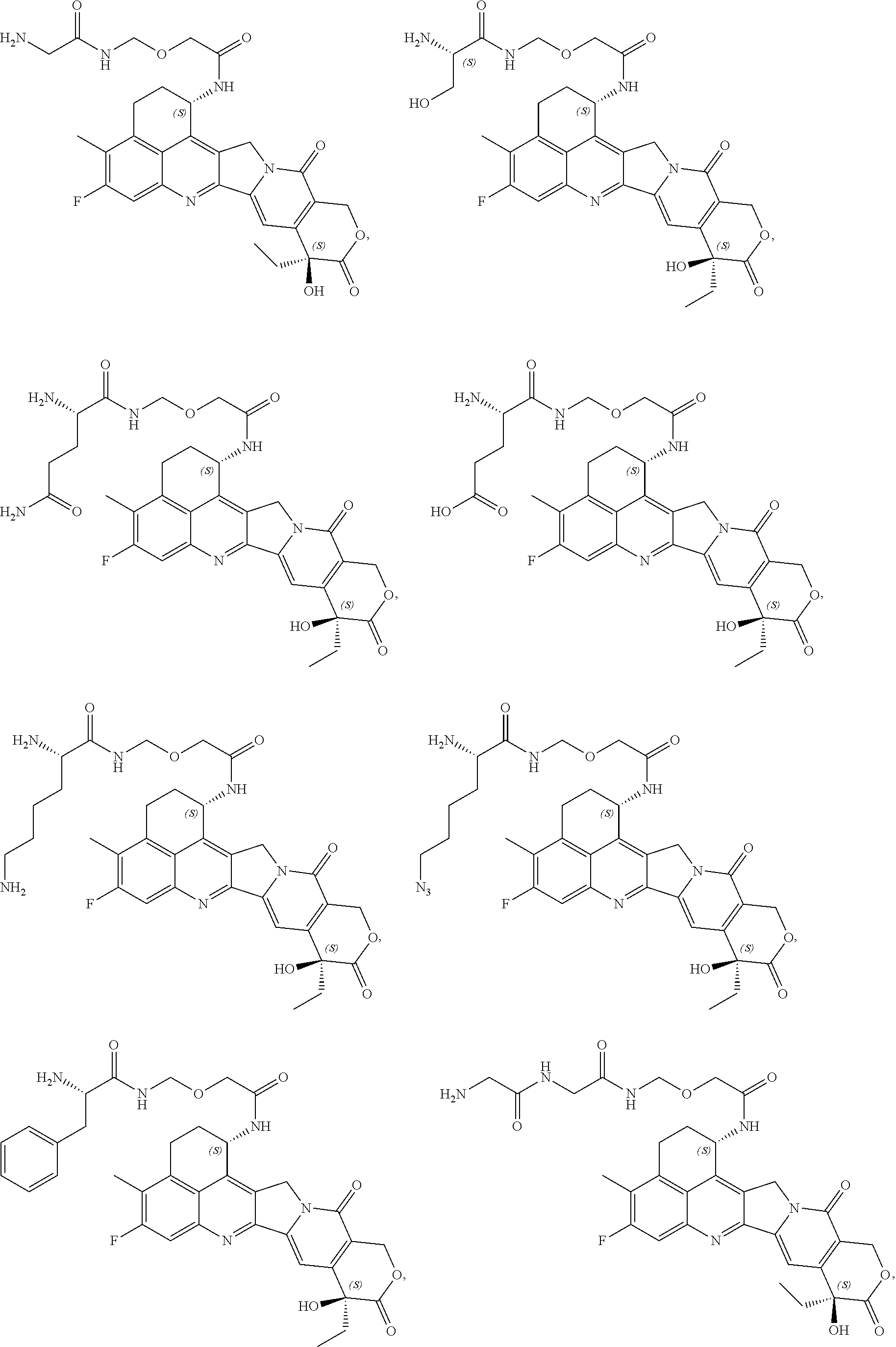

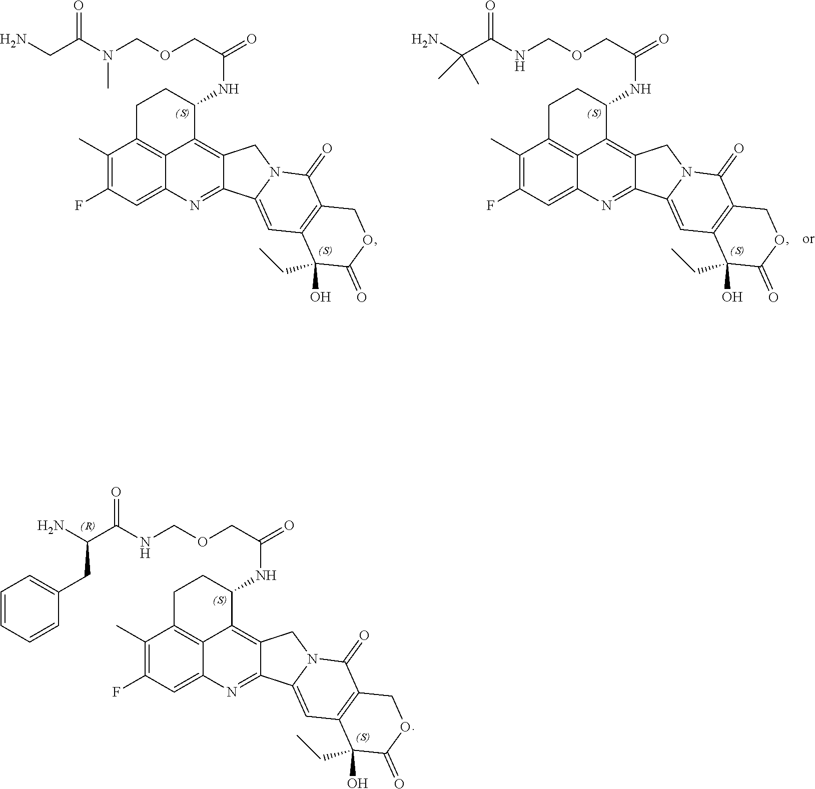

In one embodiment, said antibody or said antigen-binding fragment thereof is conjugated to a compound having a structure according to Formula (II)

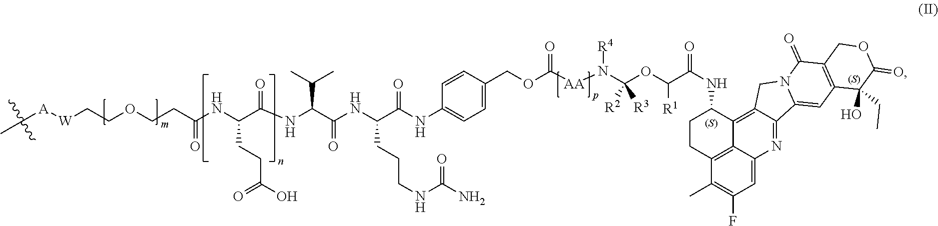

or a pharmaceutically acceptable salt thereof, wherein R¹, R², R³, and R⁴ are independently hydrogen or a C₁₋₅ alkyl;
A is a Click chemistry adduct;
W is NH, O, CO, CH₂, a phenyl, or a combination of two or more thereof;
AA is a natural or a nonnatural amino acid;
m is an integer from 0 to 8;
n is 0 or 1;
p is an integer from 1 to 6, and

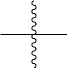

indicates the point of attachment to the antibody or the antigen-binding fragment thereof, directly or via a linker.

In one embodiment, the click chemistry adduct is a product of a copper-free click chemistry reaction selected from: (a) strain-promoted azide/dibenzocyclooctyne-amine (DBCO) click chemistry; (b) inverse electron demand Diels-Alder (IED-DA) tetrazine/trans-cyclooctene (TCO) click chemistry; (c) inverse electron demand Diels-Alder (IED-DA) tetrazine/norbonene click chemistry; (d) Diels-Alder maleimide/furan click-chemistry; (e) Staudinger ligation; and (f) nitrile-oxide/norbonene cycloaddition click chemistry.

In one embodiment, the click chemistry adduct comprises a triazole or a diazine.

In one embodiment, the click chemistry adduct is selected from the group consisting of:

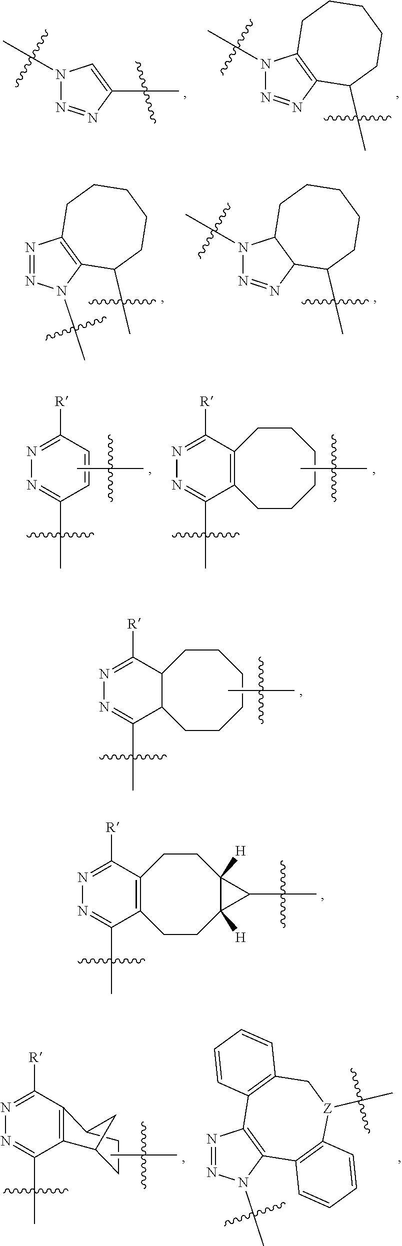

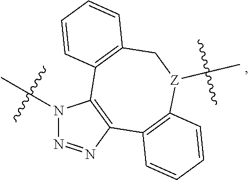

and any regio-isomers or entantiomers thereof, where R′ is H or a C₁₋₃ alkyl and Z is C or N.

In one embodiment, AA comprises a natural amino acid selected from glycine, alanine, valine, leucine, isoleucine, phenylalanine, tyrosine, threonine, lysine, asparagine, glutamine, aspartic acid, and glutamic acid.

In one embodiment, AA comprises a nonnatural amino acid selected from the group consisting of an R-amino acid, an N-methyl amino acid,

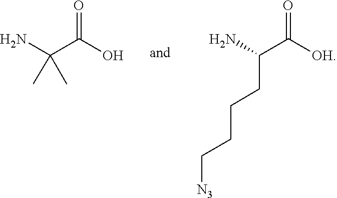

In one embodiment, said compound of Formula (II) comprises

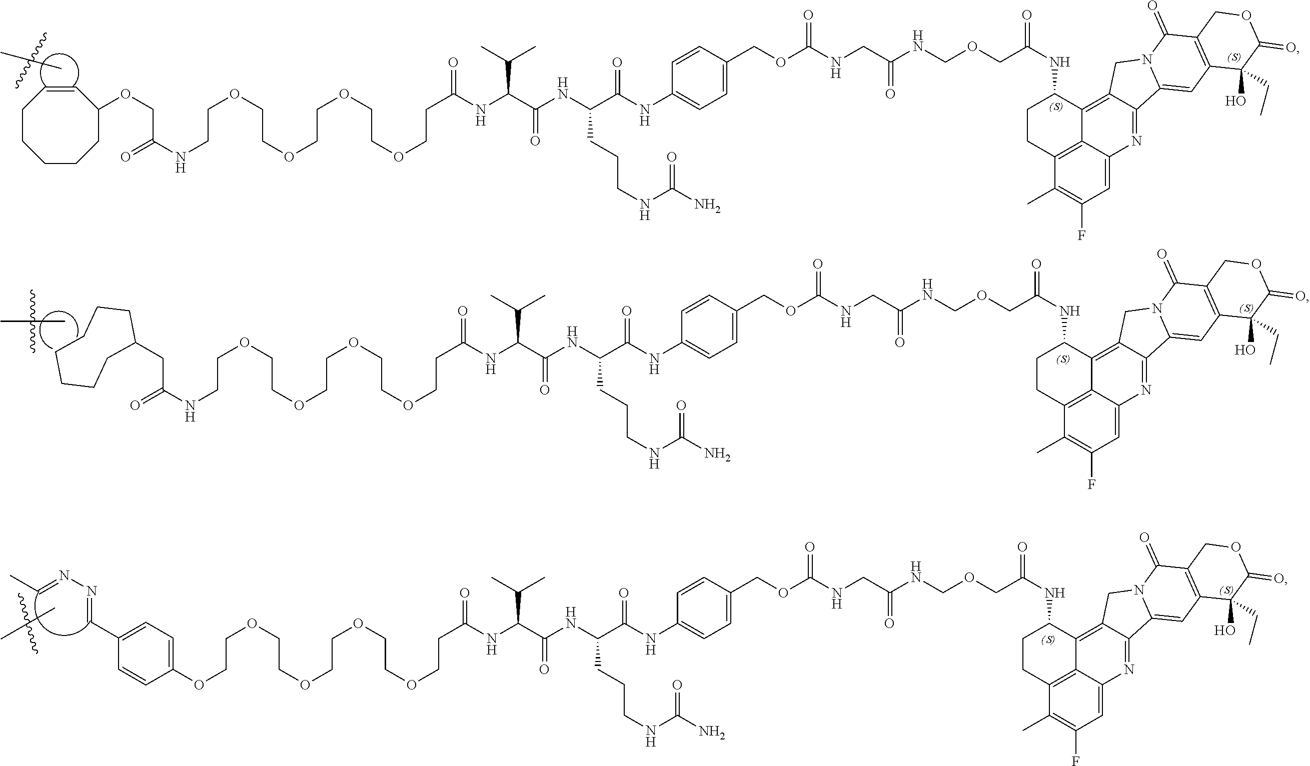

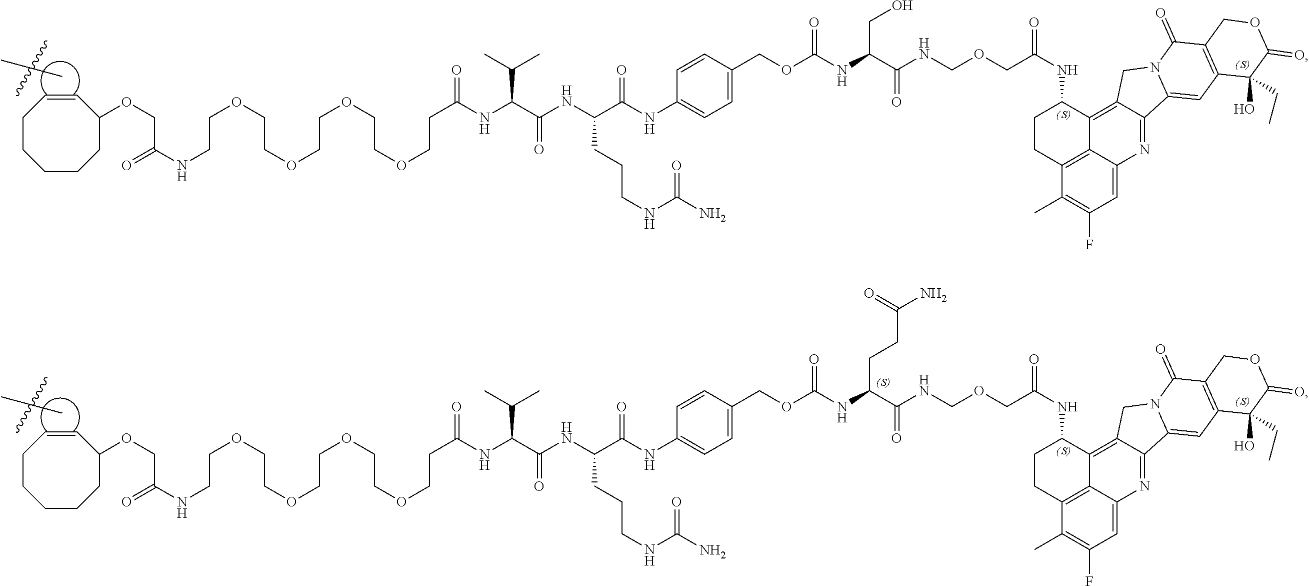

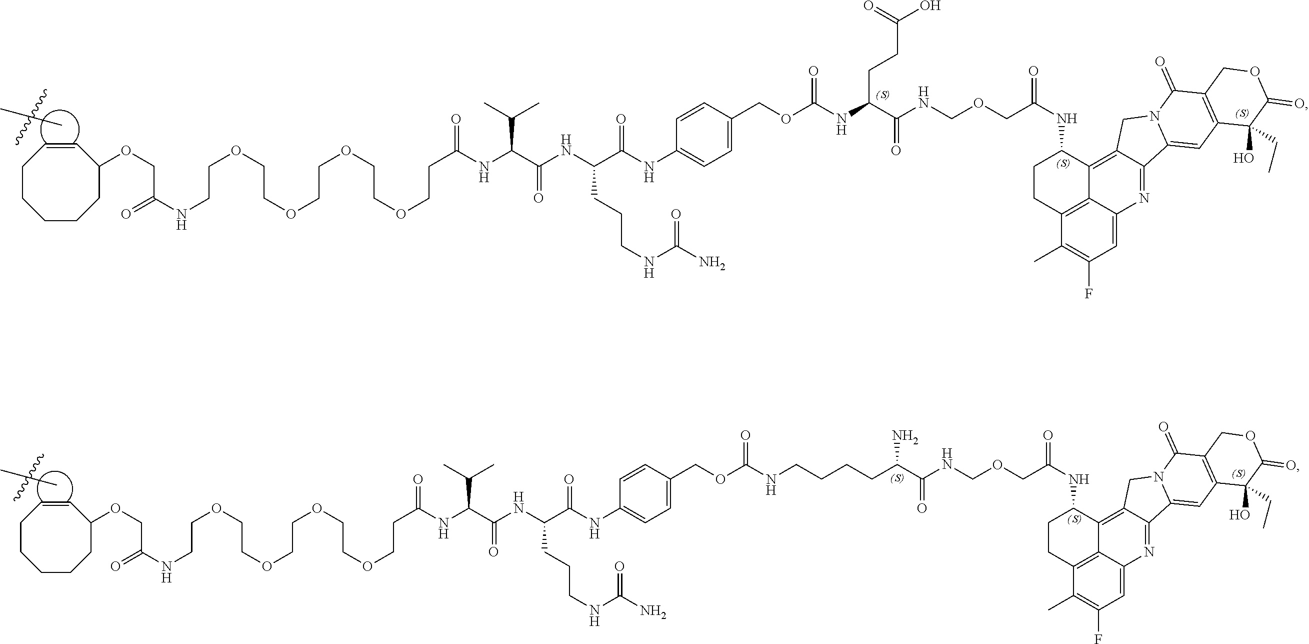

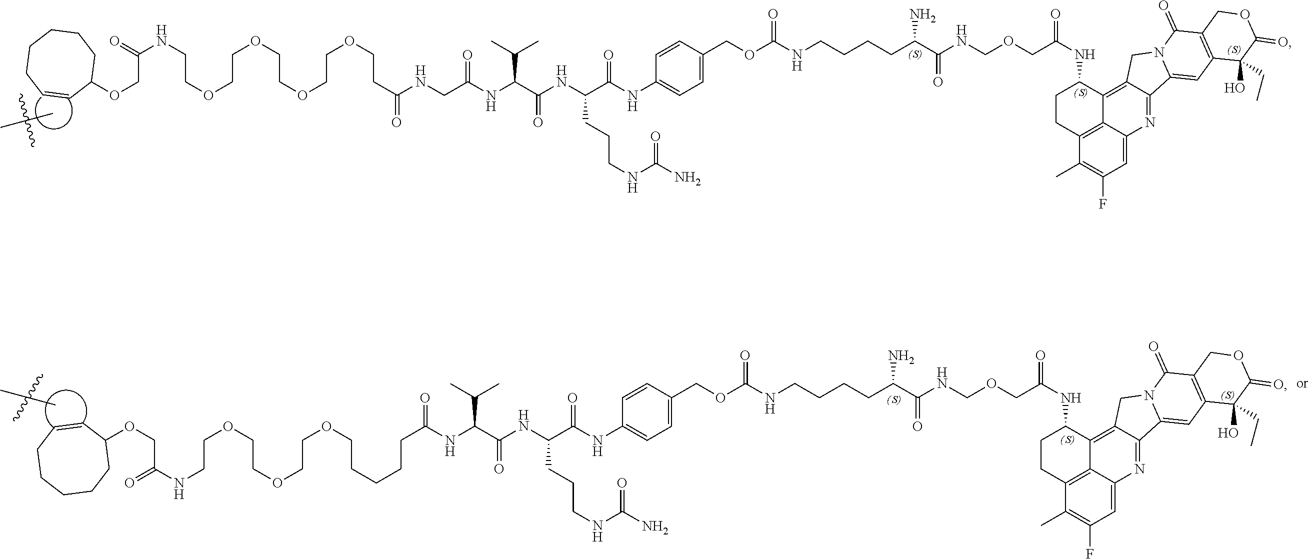

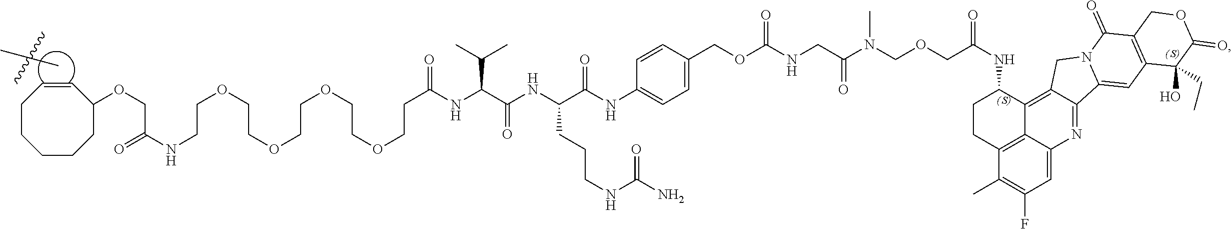

In one embodiment, said compound of Formula (II) comprises

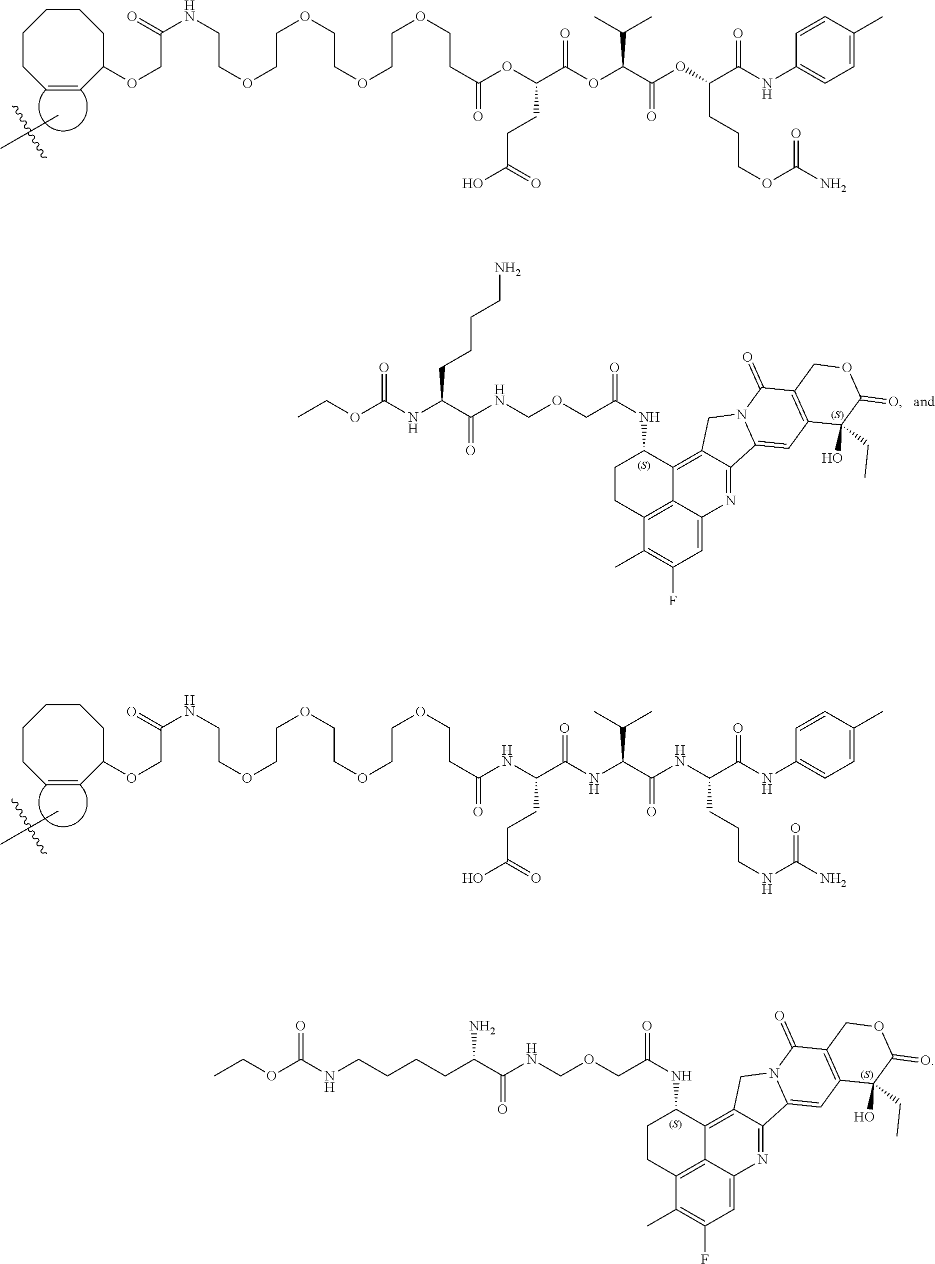

In another aspect, the present disclosure provides an antibody-drug conjugate having a structure according to Formula (III)

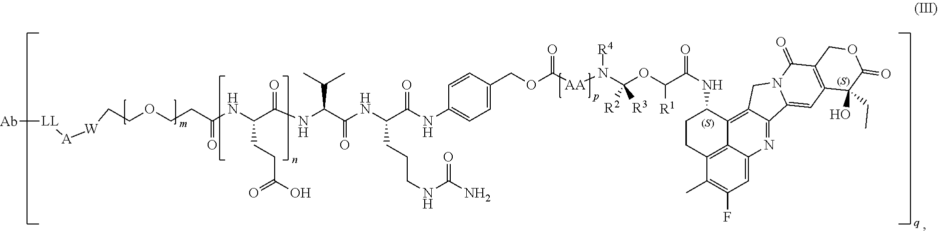

or a pharmaceutically acceptable salt thereof, wherein
Ab is an antibody or an antigen-binding fragment thereof;
R¹, R², R³, and R⁴ are independently hydrogen or a C₁₋₅ alkyl;
A is a Click chemistry adduct;
LL is a linker or a bond connecting said Ab and said A;
AA is a natural or a nonnatural amino acid;
m is an integer from 0 to 8;
n is 0 or 1;
p is an integer from 1 to 6;
and q is an integer from 1 to 10.

In another aspect, the present disclosure provides an antibody-drug conjugate having a structure according to Formula (IVa or IVb)

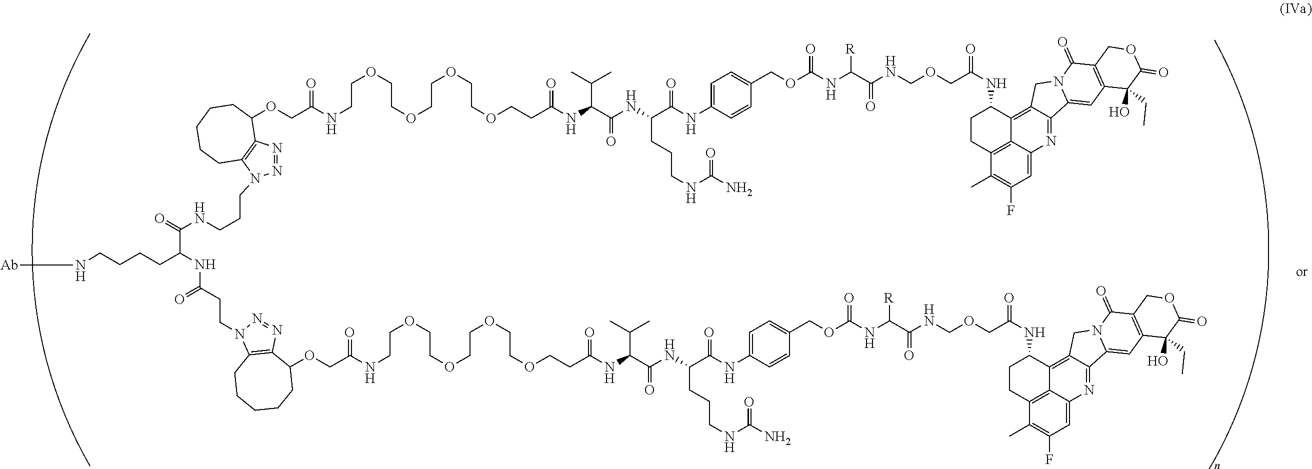

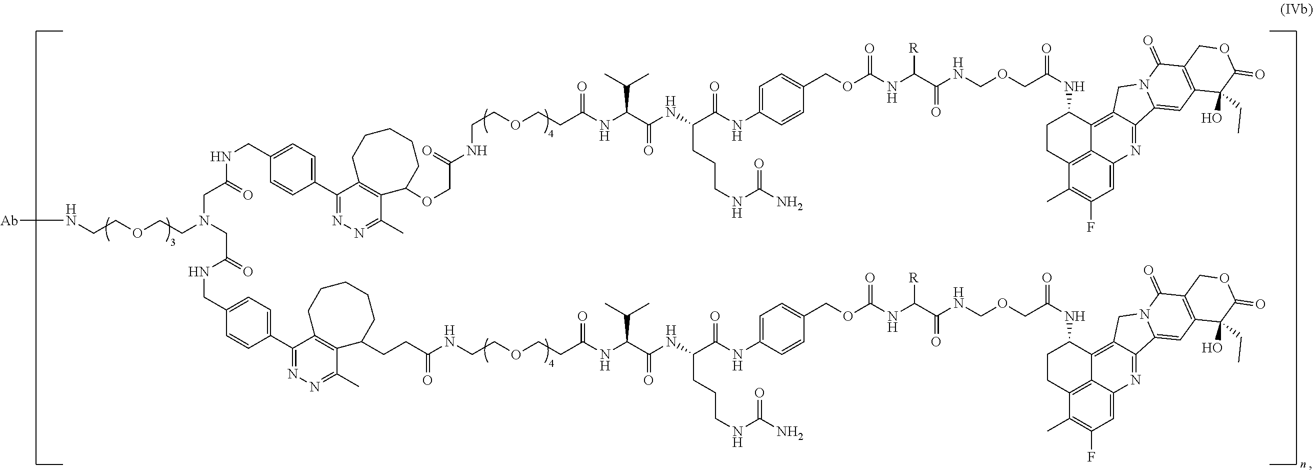

or a pharmaceutically acceptable salt thereof, wherein
Ab is an antibody or an antigen-binding fragment thereof;
R is a side chain of any natural or nonnatural amino acid;
and n is an integer from 1 to 5.

In another aspect, the present disclosure provides an antibody-drug conjugate having a structure according to Formula (IVc, IVd, IVe, IVf, IVg, IVh, IVi, IVj, or IVk)

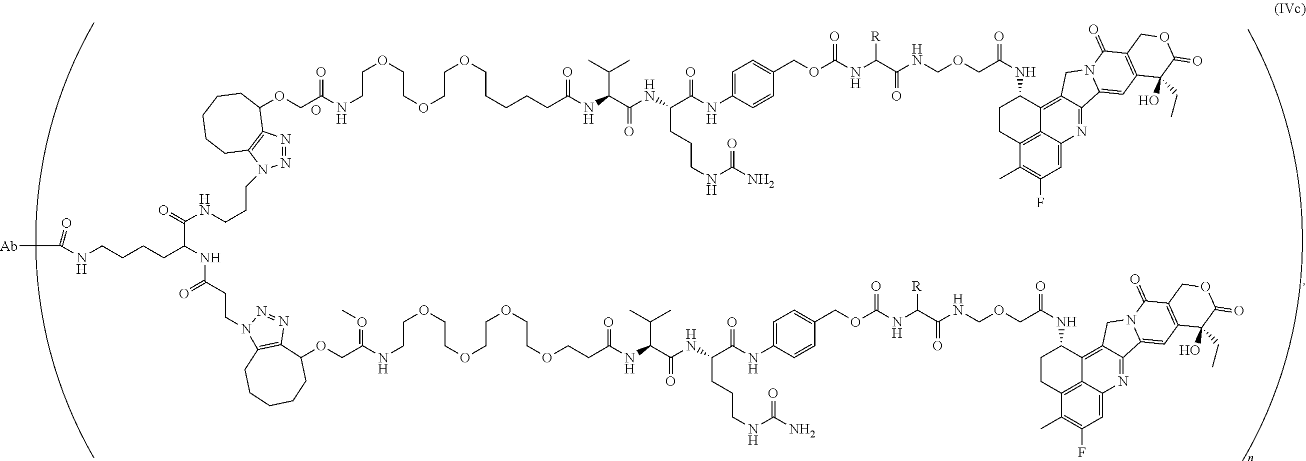

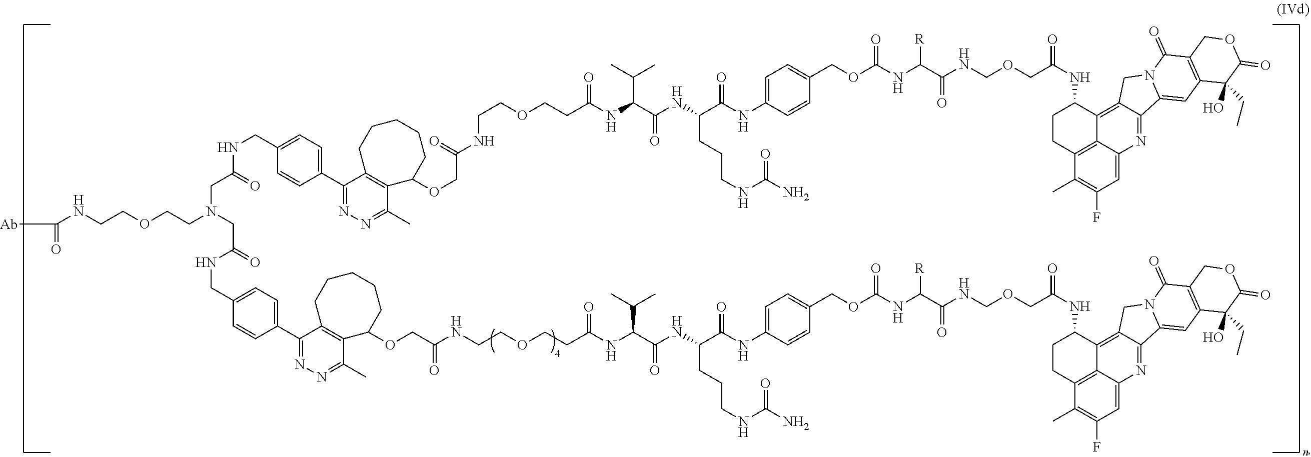

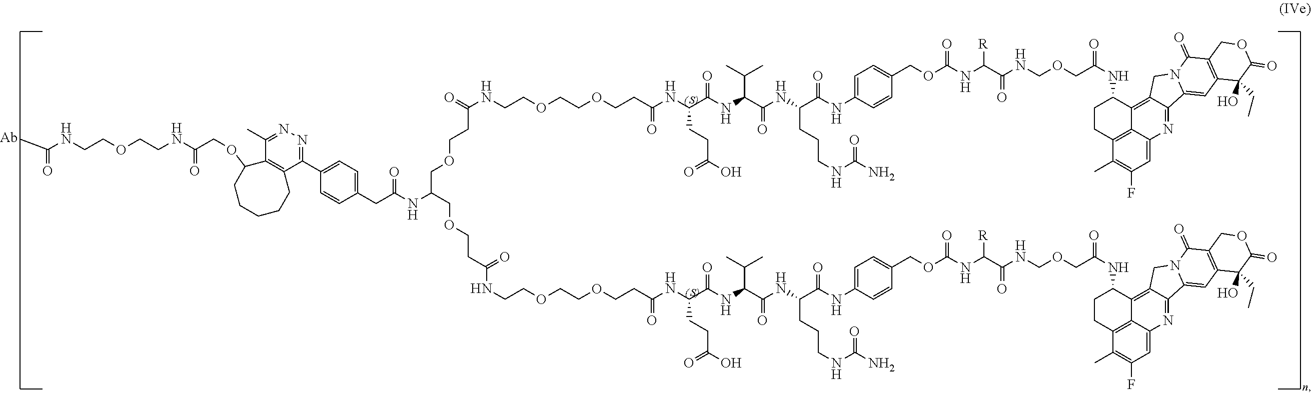

or a pharmaceutically acceptable salt thereof, wherein
Ab is an antibody or an antigen-binding fragment thereof;
R is a side chain of any natural or nonnatural amino acid;
and n is an integer from 1 to 5.

In one embodiment, said antibody or said antigen-binding fragment thereof comprises Gln295 and/or Gln297, and wherein the drug payload is conjugated to said antibody or antigen-binding fragment through the side chains of Gln295 and/or Gln297.

In one embodiment, said antibody or said antigen-binding fragment thereof is selected from an anti-HER2 antibody, an anti-STEAP2 antibody, an anti-MET antibody, an anti-EGFRVIII antibody, an anti-MUC16 antibody, an anti-PRLR antibody, an anti-PSMA antibody, an anti-FGFR2 antibody, an anti-FOLR1 antibody, an anti-HER2/HER2 bispecific antibody, an anti-MET/MET bispecific antibody, or an antigen-binding fragment thereof.

In one embodiment, the antibody or antigen-binding fragment thereof is an anti-HER2/HER2 bispecific antibody.

In one embodiment, the anti-HER2/HER2 bispecific antibody comprises:
a first antigen-binding domain (D1); and
a second antigen-binding domain (D2);
wherein D1 specifically binds a first epitope of human HER2; and
wherein D2 specifically binds a second epitope of human HER2.

In one embodiment, said antibody and linker-drug payload is conjugated site-specifically by using a transglutaminase.

In one embodiment, said transglutaminase is a microbial transglutaminase.

In another aspect, the present disclosure provides a pharmaceutical composition comprising an antibody-drug conjugate according to any one the above embodiments, co-formulated together with one or more pharmaceutically acceptable diluents, excipients, and/or additives.

In another aspect, the present disclosure provides a composition comprising a population of the antibody-drug conjugates according to any one of the above embodiments, having a drug-antibody ratio (DAR) of about 0.5 to about 30.0.

In one embodiment, the composition has a DAR of about 1.0 to about 2.5.

In one embodiment, the composition has a DAR of about 2.

In one embodiment, the composition has a DAR of about 3.0 to about 4.5.

In one embodiment, the composition has a DAR of about 4.

In one embodiment, the composition has a DAR of about 6.5 to about 8.5.

In one embodiment, the composition has a DAR of about 8.

In another aspect, the present disclosure provides a method for treating cancer in a subject in need thereof comprising the step of administering to the subject a therapeutically effective amount of the antibody-drug conjugate according to any one of the above embodiments, or the pharmaceutical composition of the above embodiments.

In another aspect, the present disclosure provides a process for manufacturing a linker-payload compound having the formula selected from the group consisting of (D′) to (N′):

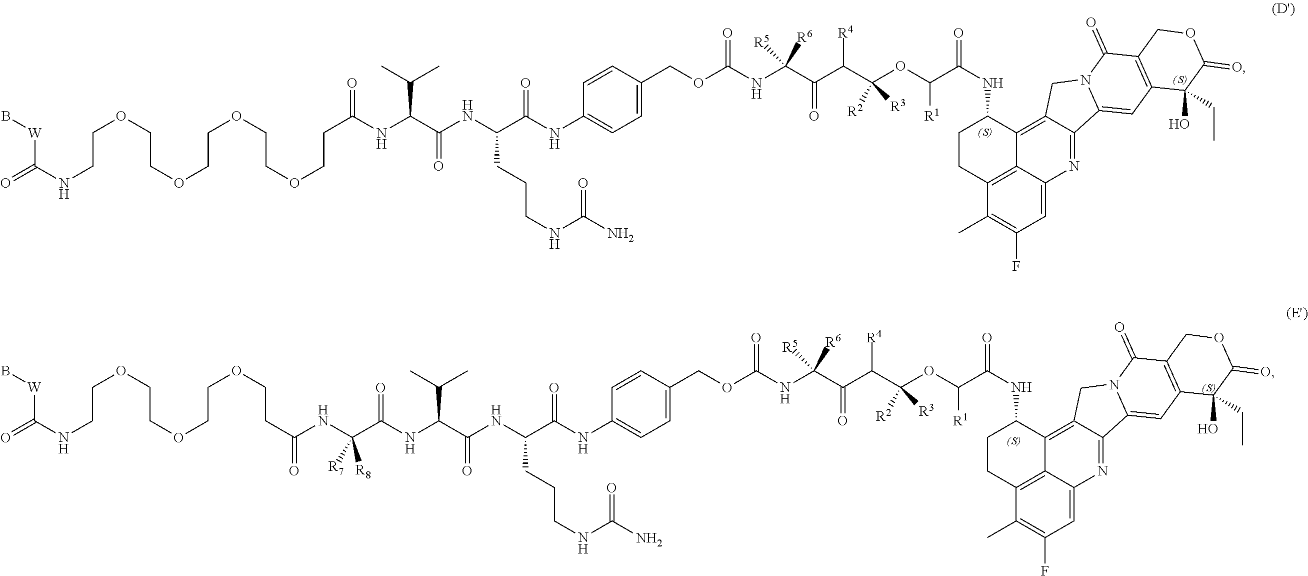

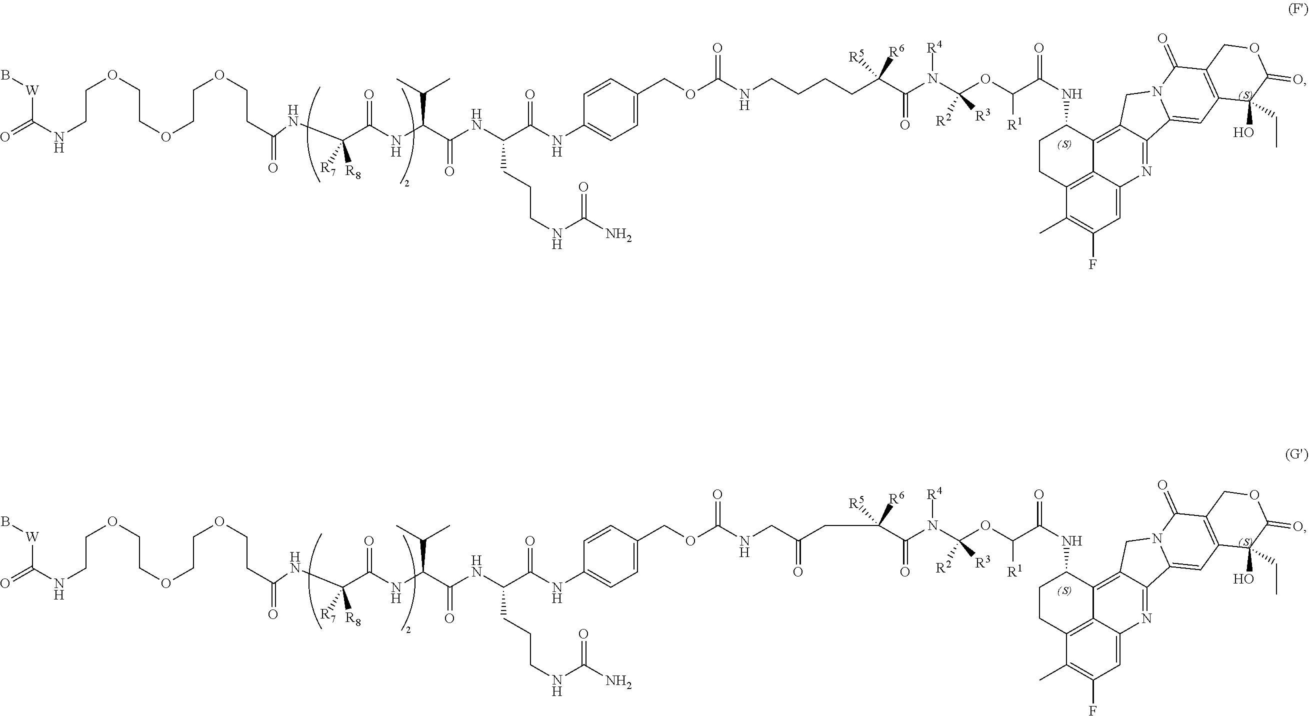

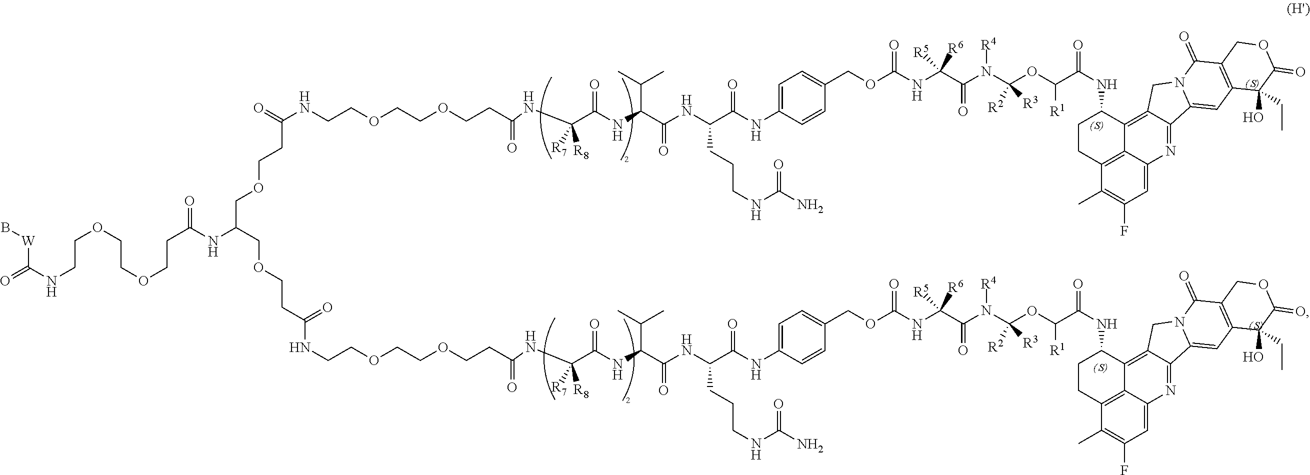

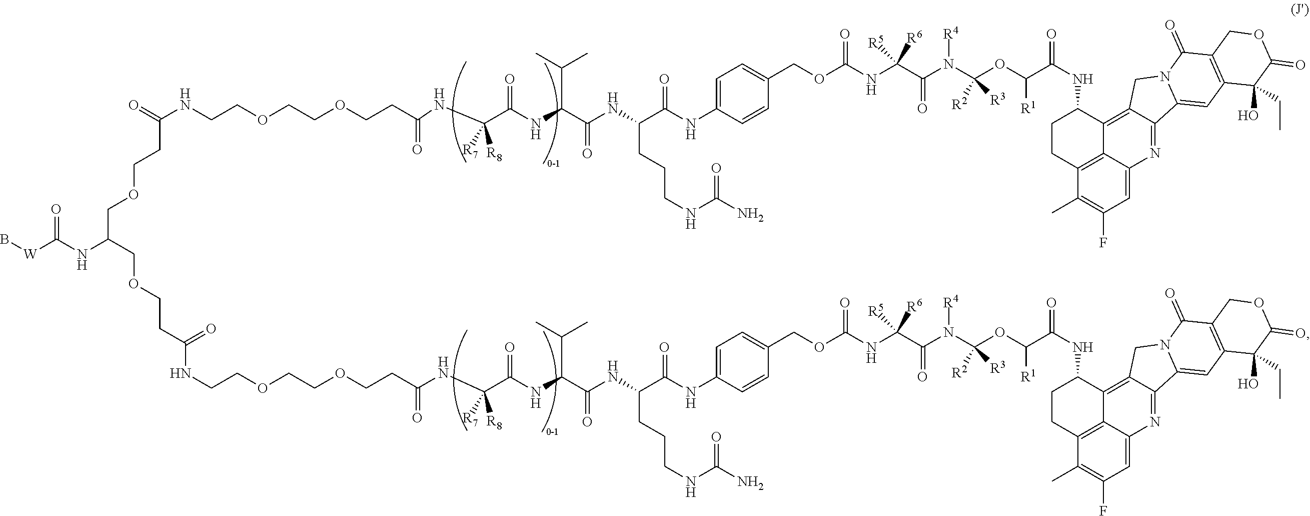

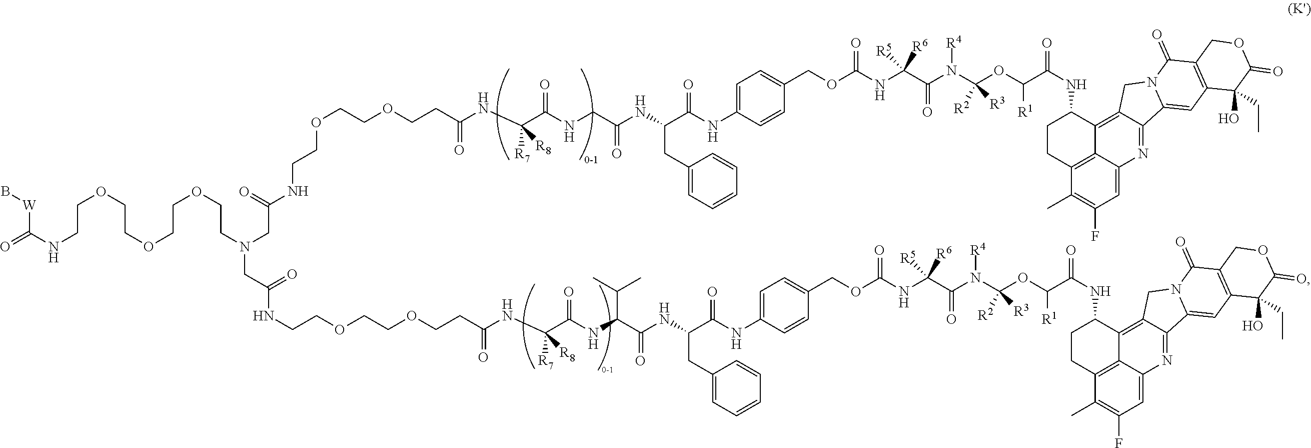

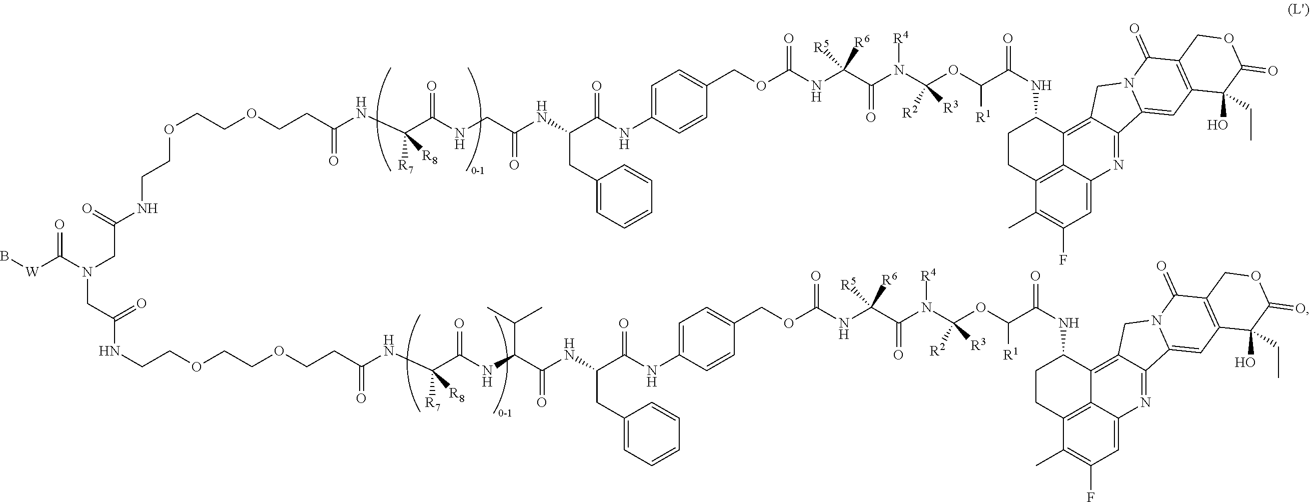

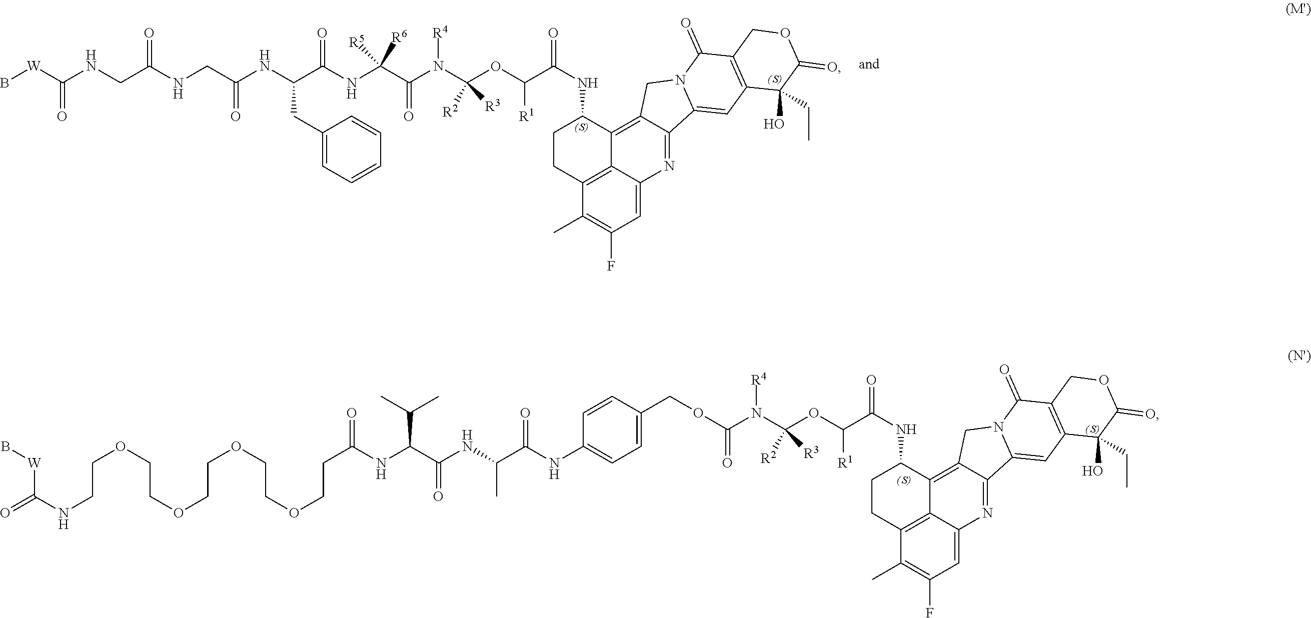

or a pharmaceutically acceptable salt thereof,
wherein R¹, R², R³, and R⁴ are independently hydrogen or a C₁₋₅ alkyl;
B is selected from the group consisting of

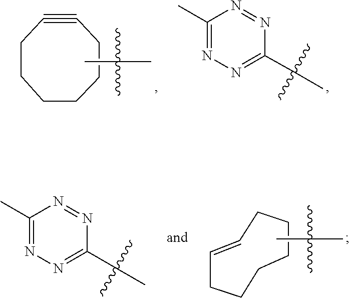

W is NH, O, CO, CH₂, a phenyl, or a combination of two or more thereof; and
R⁵, R⁶, R⁷ and R⁸ are independently hydrogen, —NH₂, or a side chain of any natural or non-natural amino acid,
the method comprising a step of exposing a payload having an amino group to an activated intermediate having a para-nitro-phenyl carbonate in the presence of a base and a coupling catalyst to afford said linker-payload compound (D′)-(G′), wherein said coupling catalyst is 4-Hydroxy-2-methylquinoline (MeHYQ).

In another aspect, the present disclosure provides a process for manufacturing a linker-payload compound having the formula (D-1)

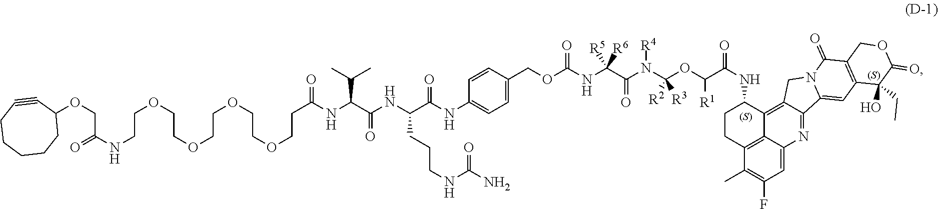

or a pharmaceutically acceptable salt thereof,
wherein R¹, R², R³, and R⁴ are independently hydrogen or a C₁₋₅ alkyl, and
R⁵ and R⁶ are independently hydrogen, —NH₂, or a side chain of any natural or non-natural amino acid,
the method comprising a step of exposing a drug payload having an amino group to an activated intermediate having a para-nitro-phenyl carbonate in the presence of a base and a coupling catalyst to afford said linker-payload compound (D), wherein said coupling catalyst is 4-Hydroxy-2-methylquinoline (MeHYQ).

In one embodiment, the activated intermediate having a para-nitro-phenyl carbonate has a structure according to formula I-I:

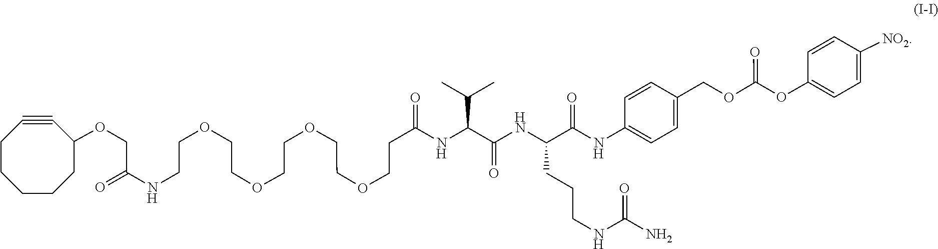

The present disclosure also relates to a process for manufacturing a linker-payload compound having the formula (D-1)

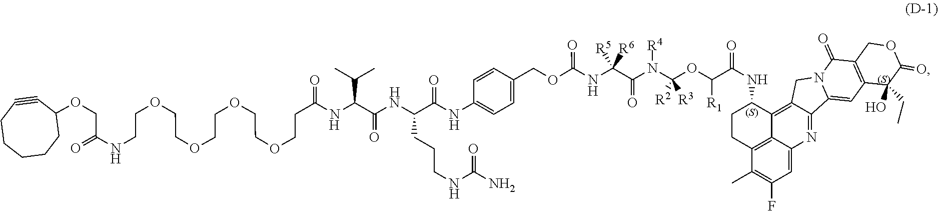

or a pharmaceutically acceptable salt thereof,
wherein R¹, R², R³, and R⁴ are independently hydrogen or a C₁₋₅ alkyl, and
R⁵ and R⁶ are independently hydrogen, —NH₂, or a side chain of any natural or non-natural amino acid,
said process comprising:
(a) providing a compound of Formula (1-1) having the structure:

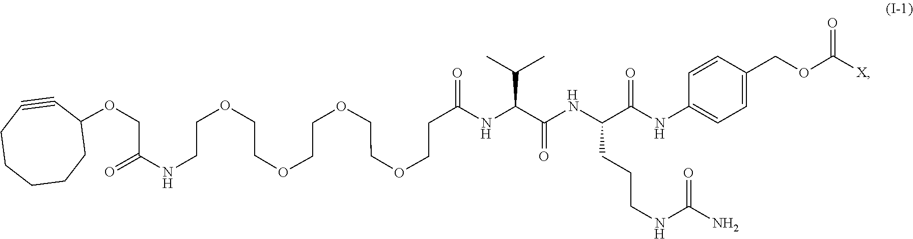

where
X is selected from the group consisting of

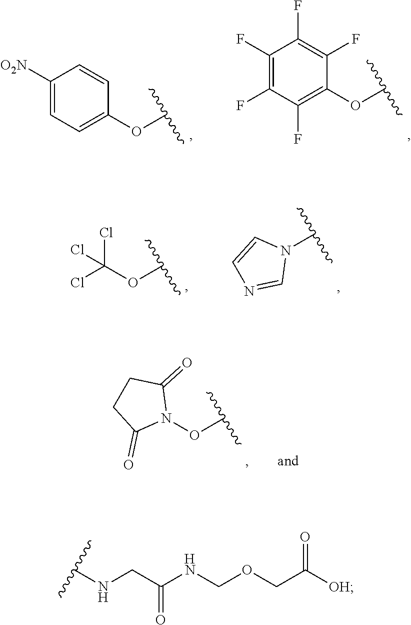

and
(b) reacting the compound of Formula (1-1) with a compound of Formula (P-I):

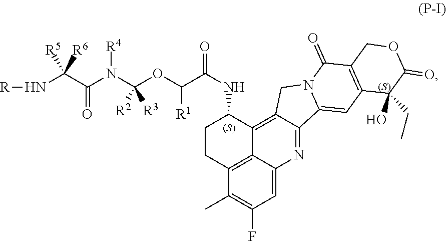

wherein
R is H or PG; and
PG is a suitable protecting group;
to produce the compound of Formula (D-1).

In one embodiment, the compound of Formula (D-1) has the following structure:

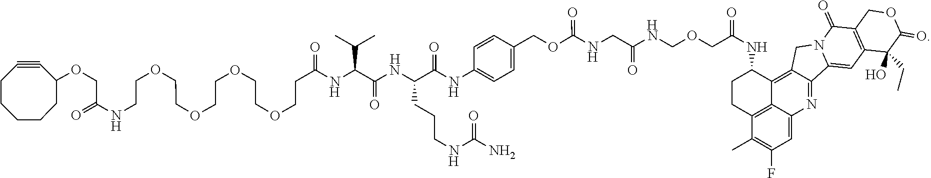

In one embodiment, the step (b) of reacting the compound of Formula (I-1) with the compound of Formula (P-I) further comprises reacting the compound of Formula (P-I), wherein R is PG, with a protecting group removing agent prior to said reacting with the compound of Formula (I-1).

In one embodiment, the PG is selected from the group consisting of allyloxycarbonyl (Alloc), benzyloxycarbonyl (Cbz), tert-butyloxycarbonyl (Boc), and 9-fluorenylmethoxycarbonyl (Fmoc).

In one embodiment, the compound of Formula (I-1) has the following structure:

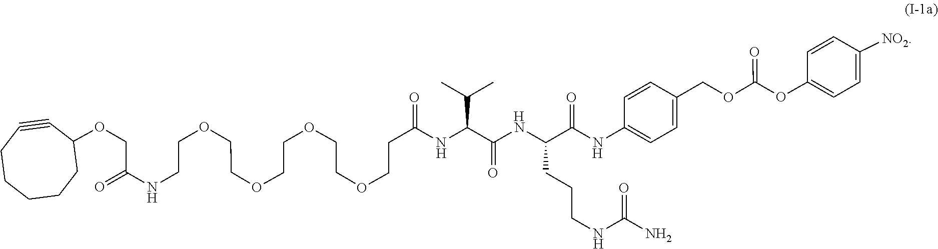

In one embodiment, the compound of Formula (P-I) has the following structure:

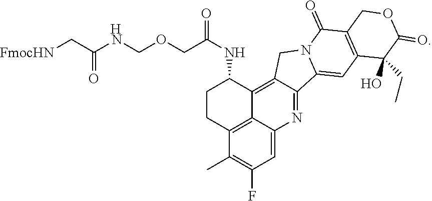

In one embodiment, the process for manufacturing a linker-payload compound having the formula (D-1) further comprises the steps of providing a compound of Formula (V) having the structure:

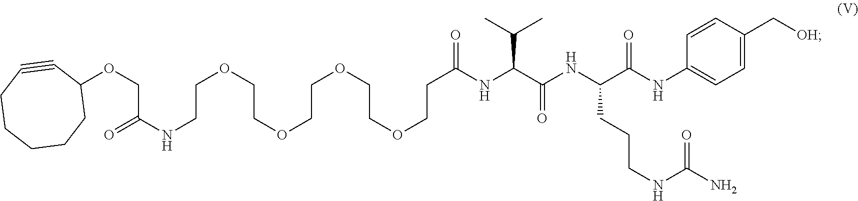 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(CO)cc1]

and forming the compound of Formula (1-1) from the compound of Formula (V) prior to the step (a).

In one embodiment, the step of forming the compound of Formula (1-1) comprises reacting the compound of Formula (V) with a compound of Formula (VIa) or Formula (VIb):

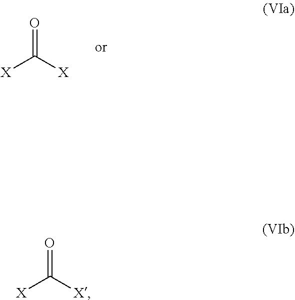

where X′ is halogen, to produce the compound of Formula (1-1).

In one embodiment, the process further comprises providing a compound of Formula (VII) having the structure:

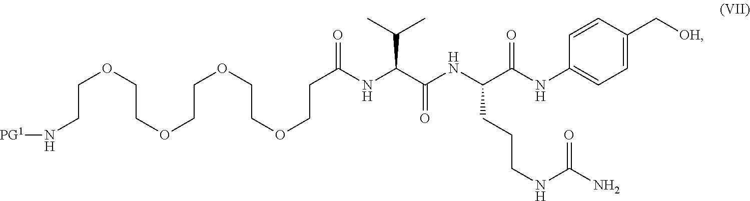

wherein PG¹ is a suitable protecting group protecting group, and forming the compound of Formula (V) from the compound of Formula (VII).

In one embodiment, the PG¹ is selected from the group consisting of allyloxycarbonyl (Alloc), benzyloxycarbonyl (Cbz), tert-butyloxycarbonyl (Boc), and 9-fluorenylmethoxycarbonyl (Fmoc).

In one embodiment, the compound of Formula (VII) has the following structure:

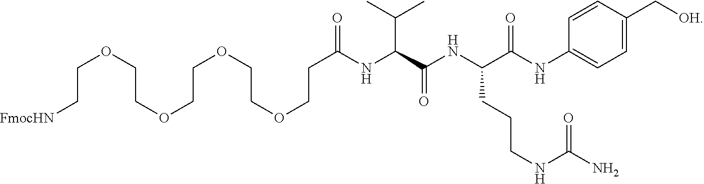

In one embodiment, the step of forming the compound of Formula (V) comprises reacting the compound of Formula (VII) with a compound of Formula (VIII):

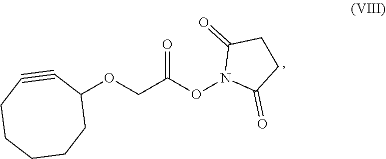 [SMILES: O=C(COC1C#CCCCCC1)ON1C(=O)CCC1=O]

to produce the compound of Formula (V).

In one embodiment, the process further comprising the steps of providing a compound of Formula (IX) having the structure:

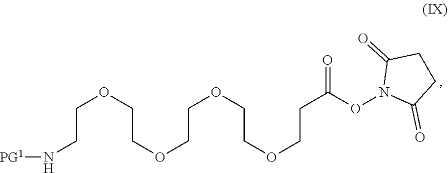

and forming the compound of Formula (VII) from the compound of Formula (IX).

In one embodiment, the compound of Formula (IX) has the following structure:

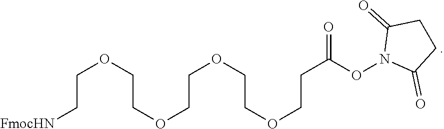

In one embodiment, the step of forming the compound of Formula (VII) comprises reacting the compound of Formula (IX) with a compound of Formula (X):

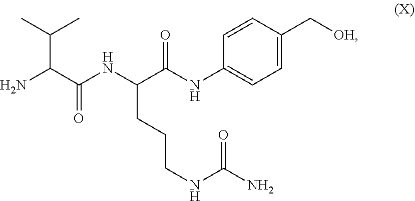 [SMILES: CC(C)C(N)C(=O)NC(CCCNC(N)=O)C(=O)Nc1ccc(CO)cc1]

to produce the compound of Formula (VII).

In one embodiment, the process further comprises the steps of providing a compound of Formula (XI) having the structure:

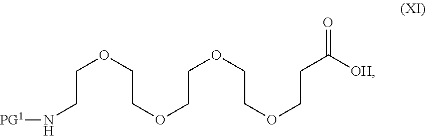

and forming the compound of Formula (IX) from the compound of Formula (XI).

In one embodiment, the compound of Formula (XI) has the following structure:

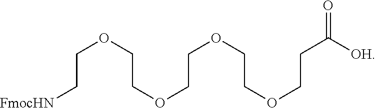

In one embodiment, the step of forming the compound of Formula (IX) comprises reacting the compound of Formula (XI) with a compound of Formula (XII):

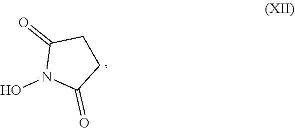

to produce the compound of Formula (IX).

In one embodiment, the process further comprises providing a compound of Formula (XIII) having the structure:

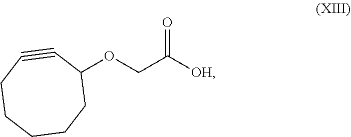 [SMILES: O=C(O)COC1C#CCCCCC1]

and
forming the compound of Formula (VIII) from the compound of Formula (XIII).

In one embodiment, the step of forming the compound of Formula (VIII) comprises reacting the compound of Formula (XIII) with a compound of Formula (XII):

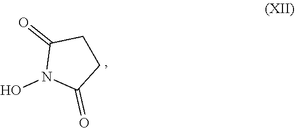

to produce the compound of Formula (VIII).

In one embodiment, the process further comprises the steps of providing a compound of Formula (XIV) having the structure:

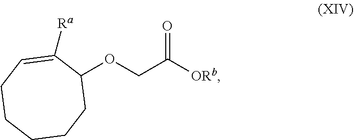

where
R^{a} is halogen; and
R^{b} is C₁₋₆ alkyl, and
forming the compound of Formula (XIII) from the compound of Formula (XIV).

In one embodiment, R^{a} is bromine.

In one embodiment, the compound of Formula (XIV) has the following structure:

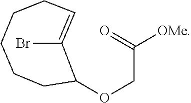 [SMILES: COC(=O)COC1CCCCC/C=C/1Br]

In one embodiment, the step of forming the compound of Formula (XIII) comprises reacting the compound of Formula (XIV) with a base to produce the compound of Formula (XIII).

In one embodiment, the base is selected from the group consisting of sodium methoxide (NaOMe), potassium tert-butoxide (t-BuOK), sodium hydride (NaH), and lithium diisopropylamide (LDA)

In one embodiment, the process further comprises the steps of providing a compound of Formula (XV) having the structure:

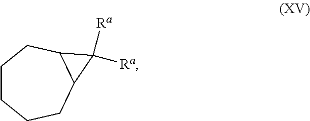

and forming the compound of Formula (XIV) from the compound of Formula (XV).

In one embodiment, the compound of Formula (XV) has the following structure:

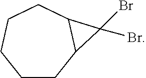

In one embodiment, the step of forming the compound of Formula (XIV) comprises reacting the compound of Formula (XV) with a compound of Formula (XVI):

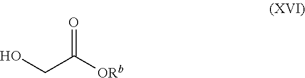

to produce the compound of Formula (XIV).

In one embodiment, the process further comprises the steps of providing a compound of Formula (XVII) having the structure:

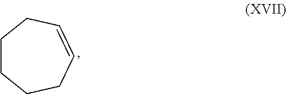

and forming the compound of Formula (XV) from the compound of Formula (XVII).

In one embodiment, the step of forming the compound of Formula (XV) comprises reacting the compound of Formula (XVII) with a bromination agent to produce the compound of Formula (XVII).

In one embodiment, the bromination agent is CHBr₃.

In one embodiment, the process further comprises the steps of providing a compound of Formula (XVIII) having the structure:

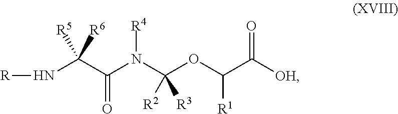

and forming the compound of Formula (P-I) from the compound of Formula (XVIII).

In one embodiment, the compound of Formula (XVIII) has the following structure:

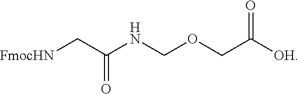

In one embodiment, the step of forming the compound of Formula (P-I) comprises reacting the compound of Formula (XVIII) with a compound of Formula (XIX):

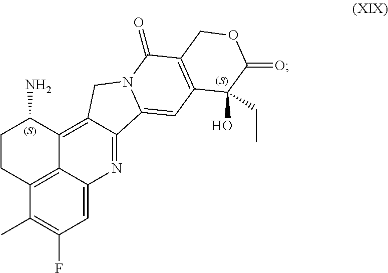

to produce the compound of Formula (P-I).

In one embodiment, the process further comprises the steps of providing a compound of Formula (XX) having the structure:

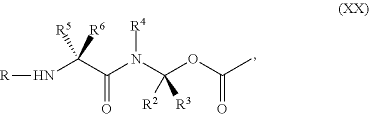

and forming the compound of Formula (XVIII) from the compound of Formula (XX).

In one embodiment, the compound of Formula (XX) has the following structure:

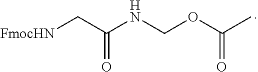

In one embodiment, the step of forming the compound of Formula (XVIII) comprises reacting the compound of Formula (XX) with a compound of Formula (XXI):

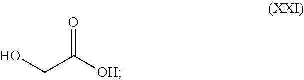

to produce the compound of Formula (XVIII).

In one embodiment, the process further comprises the steps of providing a compound of Formula (XXII) having the structure:

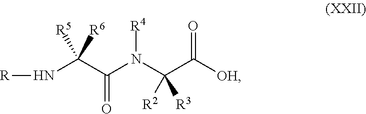

and forming the compound of Formula (XX) from the compound of Formula (XXII).

In one embodiment, the compound of Formula (XXII) has the following structure:

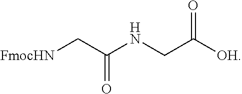

The present disclosure also relates to a process for preparation of a compound of Formula (I-1):

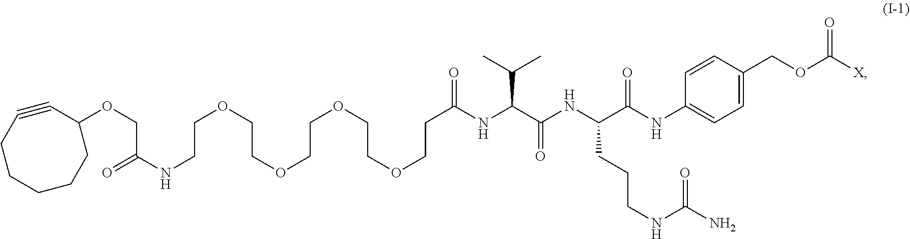

or a pharmaceutically acceptable salt thereof,
where
X is selected from the group consisting of

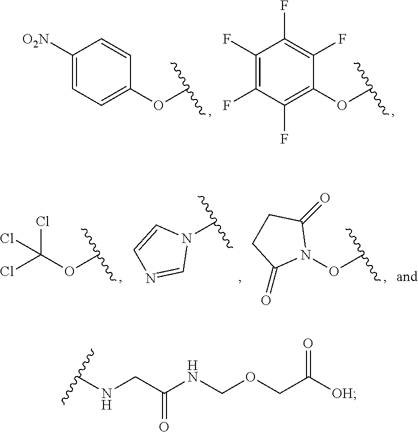

R¹, R², R³, and R⁴ are independently hydrogen or a C₁₋₅ alkyl, and
R⁵ and R⁶ are independently hydrogen, —NH₂, or a side chain of any natural or non-natural amino acid,
said process comprising:
(a) providing a compound of Formula (V) having the structure:

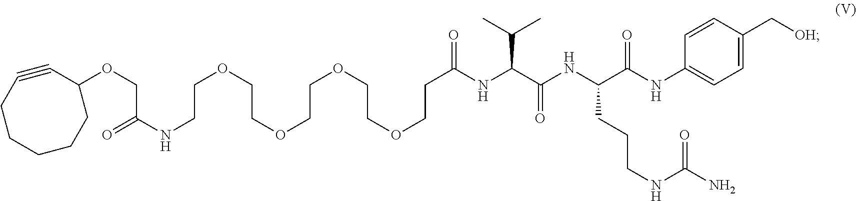 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(CO)cc1]

and
(b) forming the compound of Formula (1-1) from the compound of Formula (V).

In one embodiment, the compound of Formula (1-1) has the following structure:

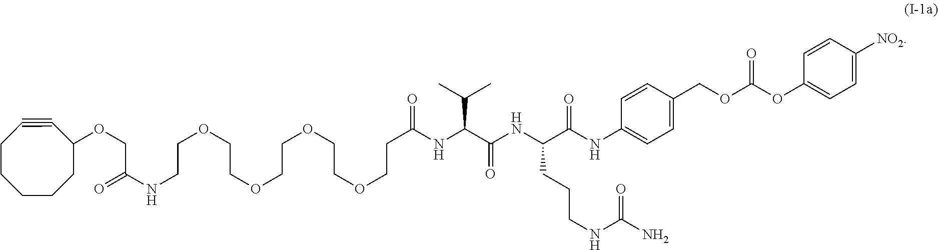 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(COC(=O)Oc2ccc([N+](=O)[O-])cc2)cc1]

In one embodiment, step (b) of forming the compound of Formula (1-1) comprises reacting the compound of Formula (V) with a compound of Formula (VIa) or Formula (VIb):

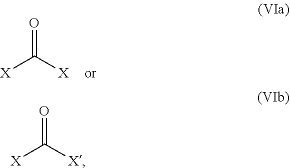

where X′ is halogen,
to produce the compound of Formula (1-1).

In one embodiment, the process further comprises the steps of providing a compound of Formula (VII) having the structure:

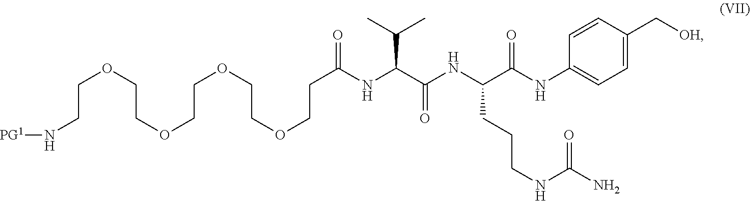

wherein PG¹ is a suitable protecting group protecting group, and forming the compound of Formula (V) from the compound of Formula (VII).

In one embodiment, the compound of Formula (VII) has the following structure:

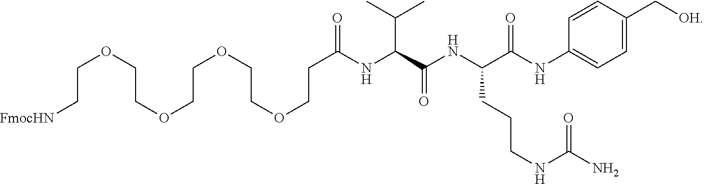

In one embodiment, the step of forming the compound of Formula (V) comprises reacting the compound of Formula (VII) with a compound of Formula (VIII):

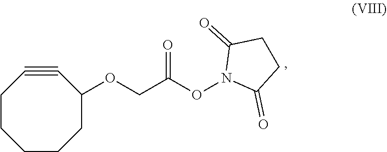 [SMILES: O=C(COC1C#CCCCCC1)ON1C(=O)CCC1=O]

to produce the compound of Formula (V).

In one embodiment, the process further comprises the steps of providing a compound of Formula (IX) having the structure:

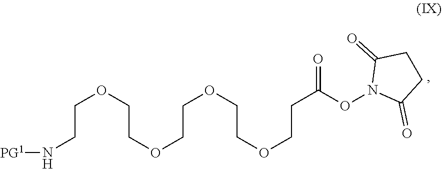

and forming the compound of Formula (VII) from the compound of Formula (IX).

In one embodiment, the compound of Formula (IX) has the following structure:

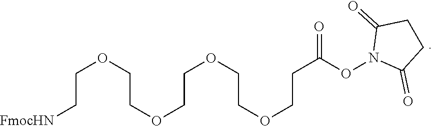

In one embodiment, the step of forming the compound of Formula (VII) comprises reacting the compound of Formula (IX) with a compound of Formula (X):

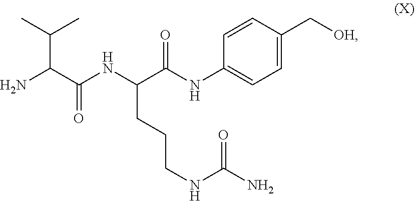 [SMILES: CC(C)C(N)C(=O)NC(CCCNC(N)=O)C(=O)Nc1ccc(CO)cc1]

to produce the compound of Formula (VII).

In one embodiment, the process further comprises the steps of providing a compound of Formula XI) having the structure:

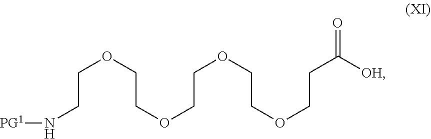

and forming the compound of Formula (IX) from the compound of Formula (XI).

In one embodiment, the compound of Formula (XI) has the following structure:

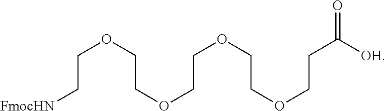

In one embodiment, the step of forming the compound of Formula (IX) comprises reacting the compound of Formula (XI) with a compound of Formula (XII):

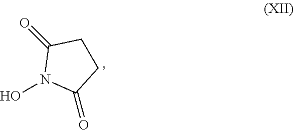

to produce the compound of Formula (IX).

The present disclosure also relates to a process for preparation of a compound of Formula (XVIII):

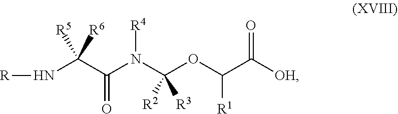

or a pharmaceutically acceptable salt thereof,
wherein R¹, R², R³, and R⁴ are independently hydrogen or a C₁₋₅ alkyl, and
R⁵ and R⁶ are independently hydrogen, —NH₂, or a side chain of any natural or non-natural amino acid. This process comprises:
(a) providing a compound of Formula (XX) having the structure:

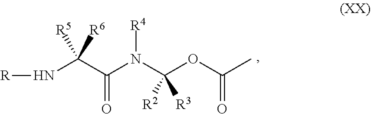

and
(b) forming the compound of Formula (XVIII) from the compound of Formula (XX).

In one embodiment, the compound of Formula (XVIII) has the following structure:

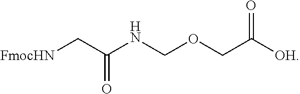

In one embodiment, the compound of Formula (XX) has the following structure:

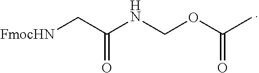

In one embodiment, the step of forming the compound of Formula (XVIII) comprises reacting the compound of Formula (XX) with a compound of Formula (XXI):

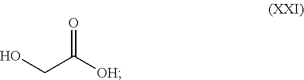

to produce the compound of Formula (XVIII).

In one embodiment, the process further comprises the steps of providing a compound of Formula (XXII) having the structure:

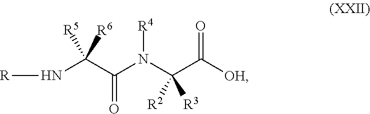

and forming the compound of Formula (XX) from the compound of Formula (XXII).

In one embodiment, the compound of Formula (XXII) has the following structure:

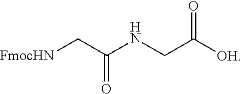

The present disclosure also relates to a process for preparation of a compound of Formula (D-1):

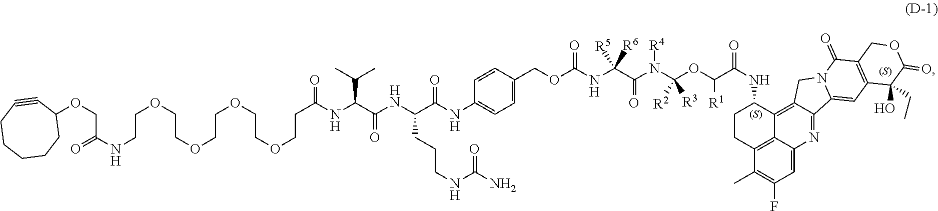

or a pharmaceutically acceptable salt thereof,
wherein R¹, R², R³, and R⁴ are independently hydrogen or a C₁₋₅ alkyl, and
R⁵ and R⁶ are independently hydrogen, —NH₂, or a side chain of any natural or non-natural amino acid. This process comprises:
(a) providing a compound of Formula (I-1) having the structure:

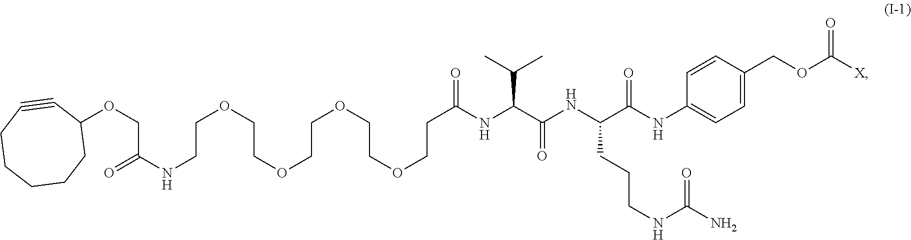

wherein
X is selected from the group consisting of

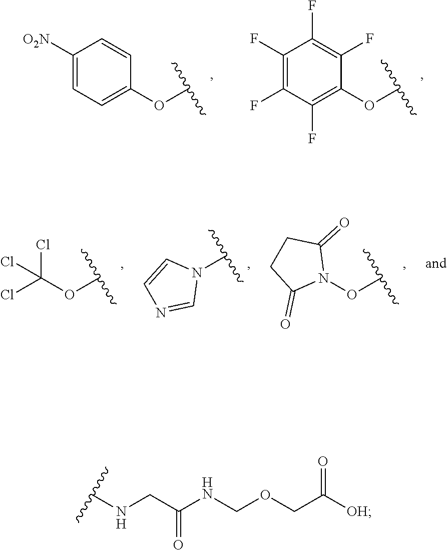

and
(b) reacting the compound of Formula (1-1) with a compound of Formula (P-I):

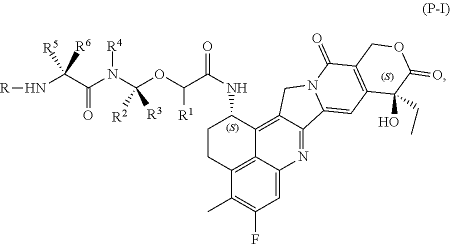

wherein
R is H or PG; and
PG is a suitable protecting group,
to produce the compound of Formula (D-1).

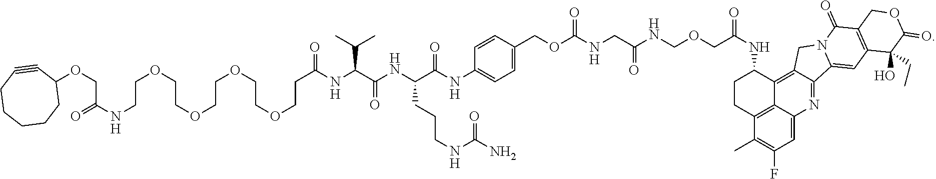 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

In one embodiment, the compound of Formula (1-1) has the following structure:

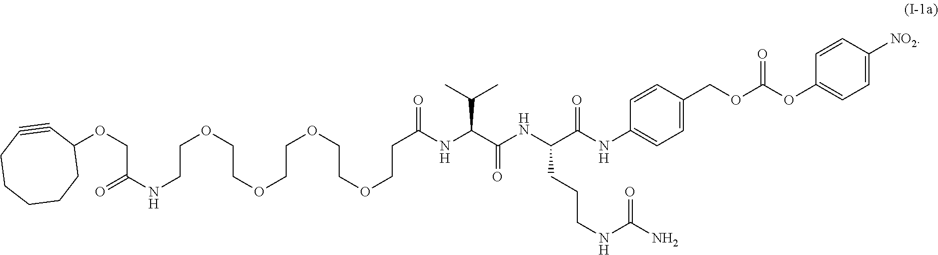 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(COC(=O)Oc2ccc([N+](=O)[O-])cc2)cc1]

In one embodiment, the step (b) of reacting the compound of Formula (1-1) with the compound of Formula (P-I) further comprises reacting the compound of Formula (P-I), wherein R is PG, with a protecting group removing agent prior to said reacting with the compound of Formula (1-1).

In one embodiment, the PG is selected from the group consisting of allyloxycarbonyl (Alloc), benzyloxycarbonyl (Cbz), tert-butyloxycarbonyl (Boc), and 9-fluorenylmethoxycarbonyl (Fmoc).

In one embodiment, the compound of Formula (P-I) has the following structure:

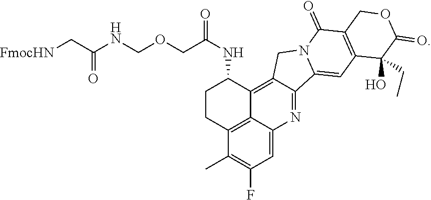

In one embodiment, the process further comprises the steps of providing a compound of Formula (XVIII) having the structure:

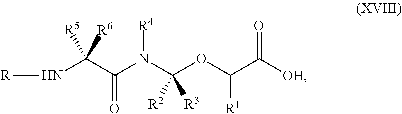

and forming the compound of Formula (P-I) from the compound of Formula (XVIII).

In one embodiment, the compound of Formula (XVIII) has the following structure:

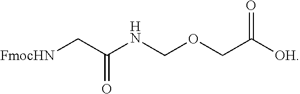

In one embodiment, the step of forming the compound of Formula (P-I) comprises reacting the compound of Formula (XVIII) with a compound of Formula (XIX):

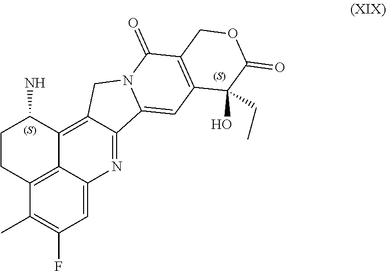

to produce the compound of Formula (P-I).

The present disclosure also relates to a process for preparation of a compound of Formula (D-1):

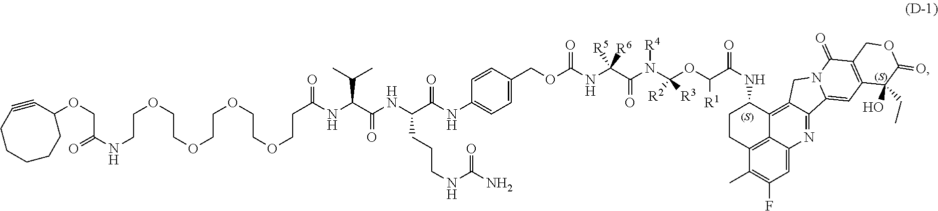

or a pharmaceutically acceptable salt thereof,
wherein R¹, R², R³, and R⁴ are independently hydrogen or a C₁₋₅ alkyl, and
R⁵ and R⁶ are independently hydrogen, —NH₂, or a side chain of any natural or non-natural amino acid,
said process comprising:
(a) providing a compound of Formula (XXIII):
   and

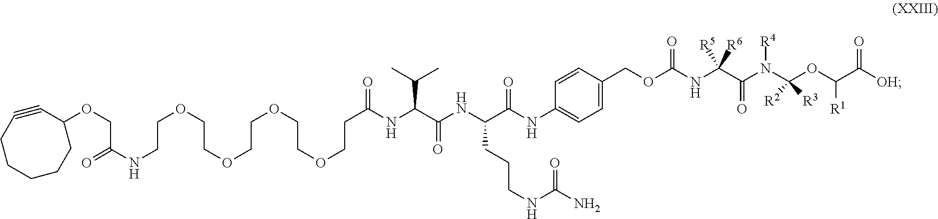

(b) reacting the compound of Formula (XXIII) with a compound having the structure:

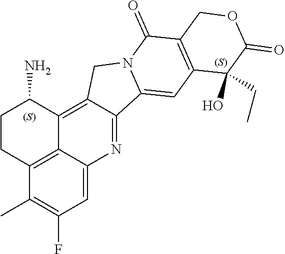

F in the presence of an activating reagent and a base to produce the compound of Formula (D-1).

In one embodiment, the compound of Formula (D-1) has the following structure:

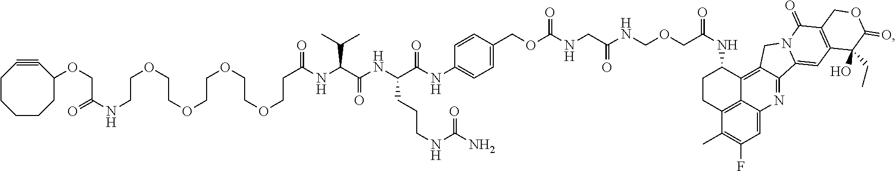 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

In one aspect, the present disclosure provides a compound of Formula (I-1):

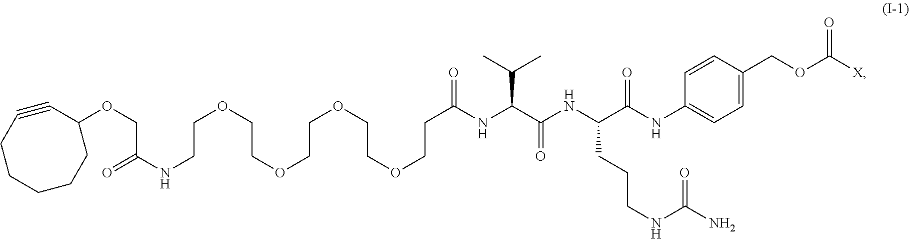

or a pharmaceutically acceptable salt thereof,
where
X is selected from the group consisting of

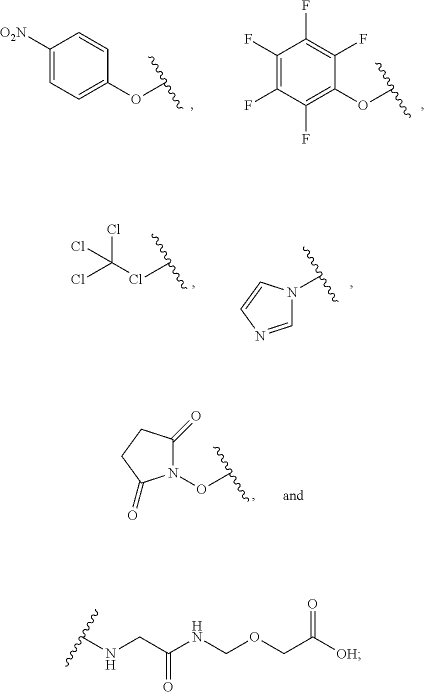

R¹, R², R³, and R⁴ are independently hydrogen or a C₁₋₅ alkyl, and
R⁵ and R⁶ are independently hydrogen, —NH₂, or a side chain of any natural or non-natural amino acid.

In one embodiment, the compound of Formula (1-1) has the following structure:

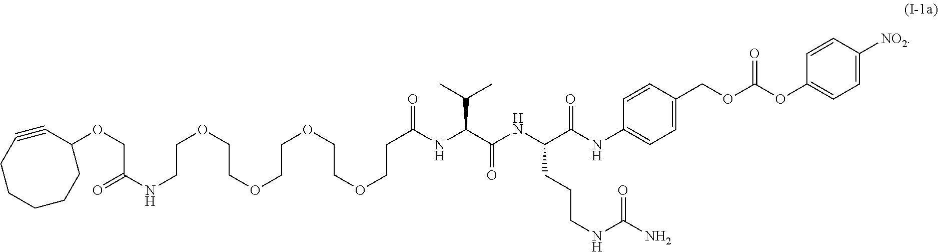 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(COC(=O)Oc2ccc([N+](=O)[O-])cc2)cc1]

In one aspect, the present disclosure provides a compound of Formula (XVIII):

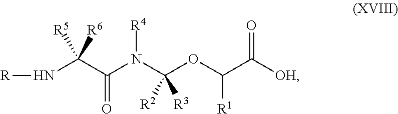

or a pharmaceutically acceptable salt thereof,
wherein R¹, R², R³, and R⁴ are independently hydrogen or a C₁₋₅ alkyl, and
R⁵ and R⁶ are independently hydrogen, —NH₂, or a side chain of any natural or non-natural amino acid.

In one embodiment, the compound of Formula (XVIII) has the following structure:

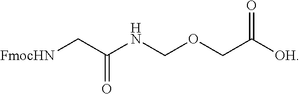

In another aspect, the present disclosure provides a linker-payload compound of formula (D),

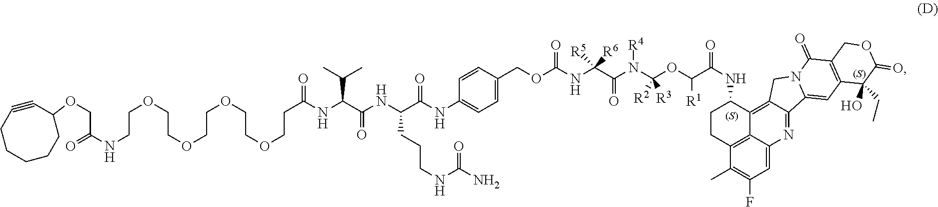

or a pharmaceutically acceptable salt thereof, wherein R¹, R², R³, and R⁴ are independently hydrogen or a C₁₋₅ alkyl, and
R⁵ and R⁶ are independently hydrogen, —NH₂, or a side chain of any natural or non-natural amino acid.

In another aspect, the present disclosure provides a linker-payload compound having the formula selected from the group consisting of (D′) to (N′):

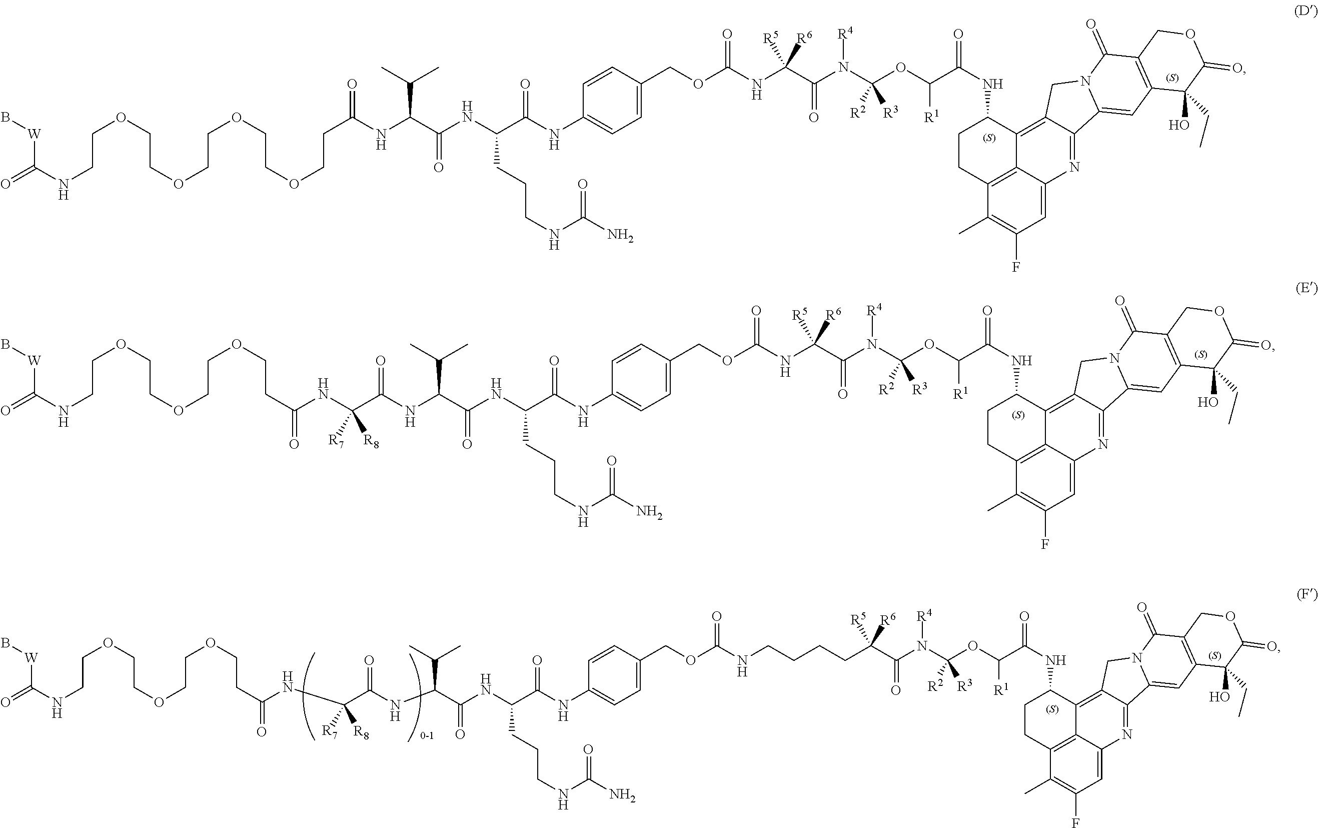

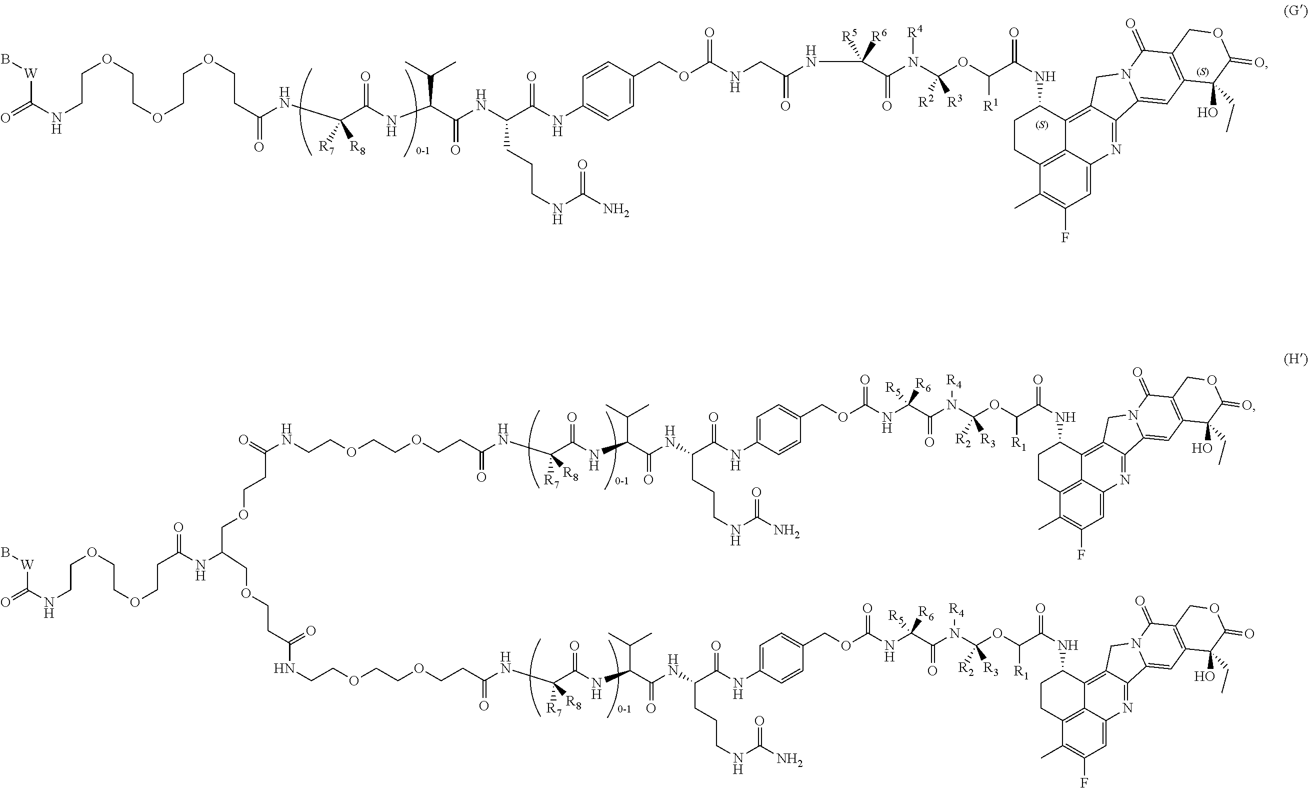

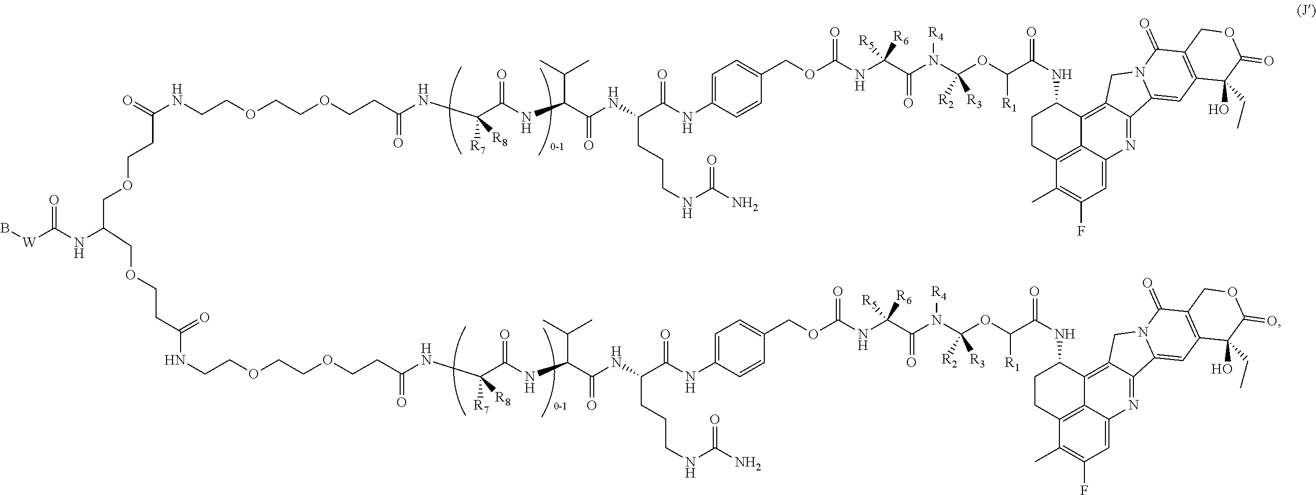

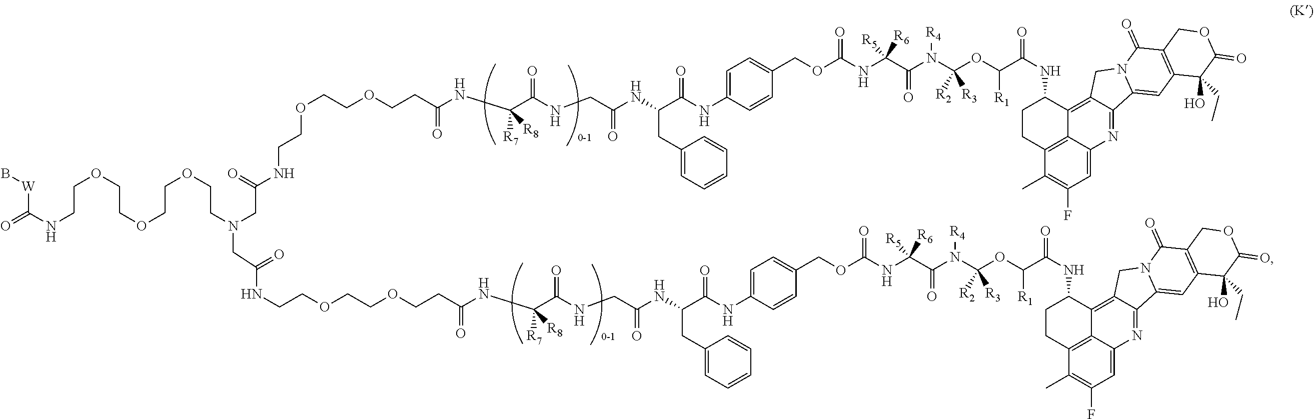

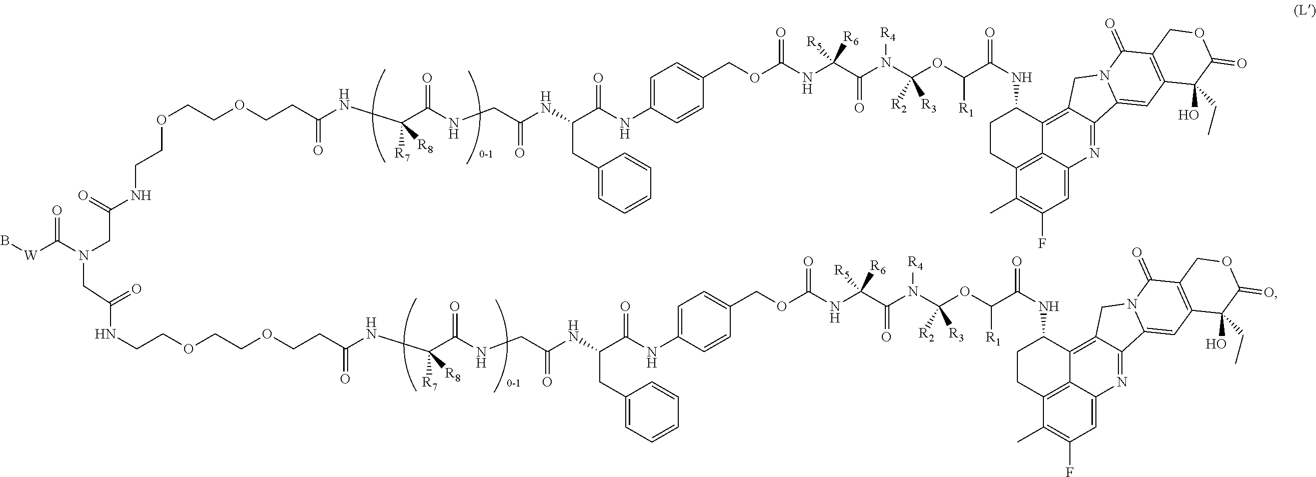

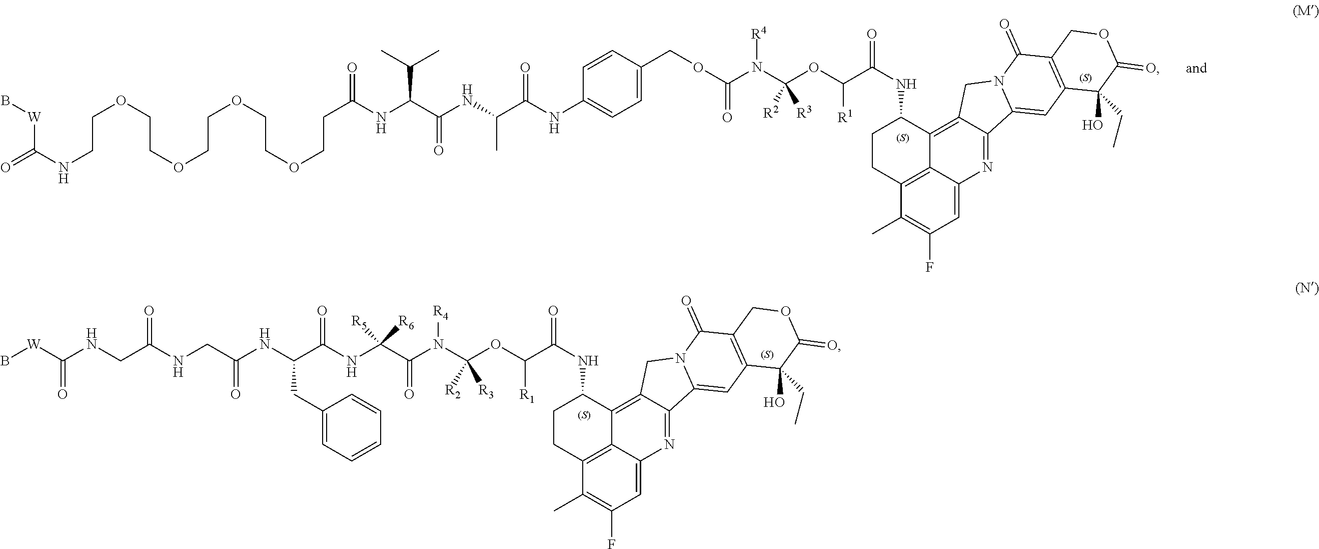

or a pharmaceutically acceptable salt thereof,
wherein R¹, R², R³, and R⁴ are independently hydrogen or a C₁₋₅ alkyl;
B is selected from the group consisting of

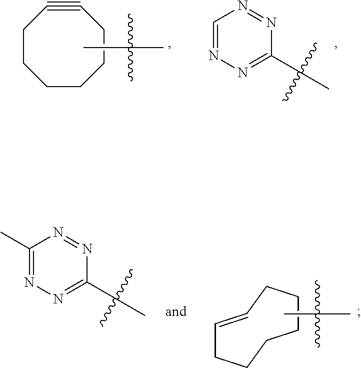

W is NH, O, CO, CH₂, a phenyl, or a combination of two or more thereof; and
R⁵, R⁶, R⁷ and R⁸ are independently hydrogen, —NH₂, or a side chain of any natural or non-natural amino acid,
the method comprising a step of exposing a payload having an amino group to an activated intermediate having a para-nitro-phenyl carbonate in the presence of a base and a coupling catalyst to afford said linker-payload compound (D′)-(G′), wherein said coupling catalyst is 4-Hydroxy-2-methylquinoline (MeHYQ).

In one embodiment, the structure is selected from the group consisting of:

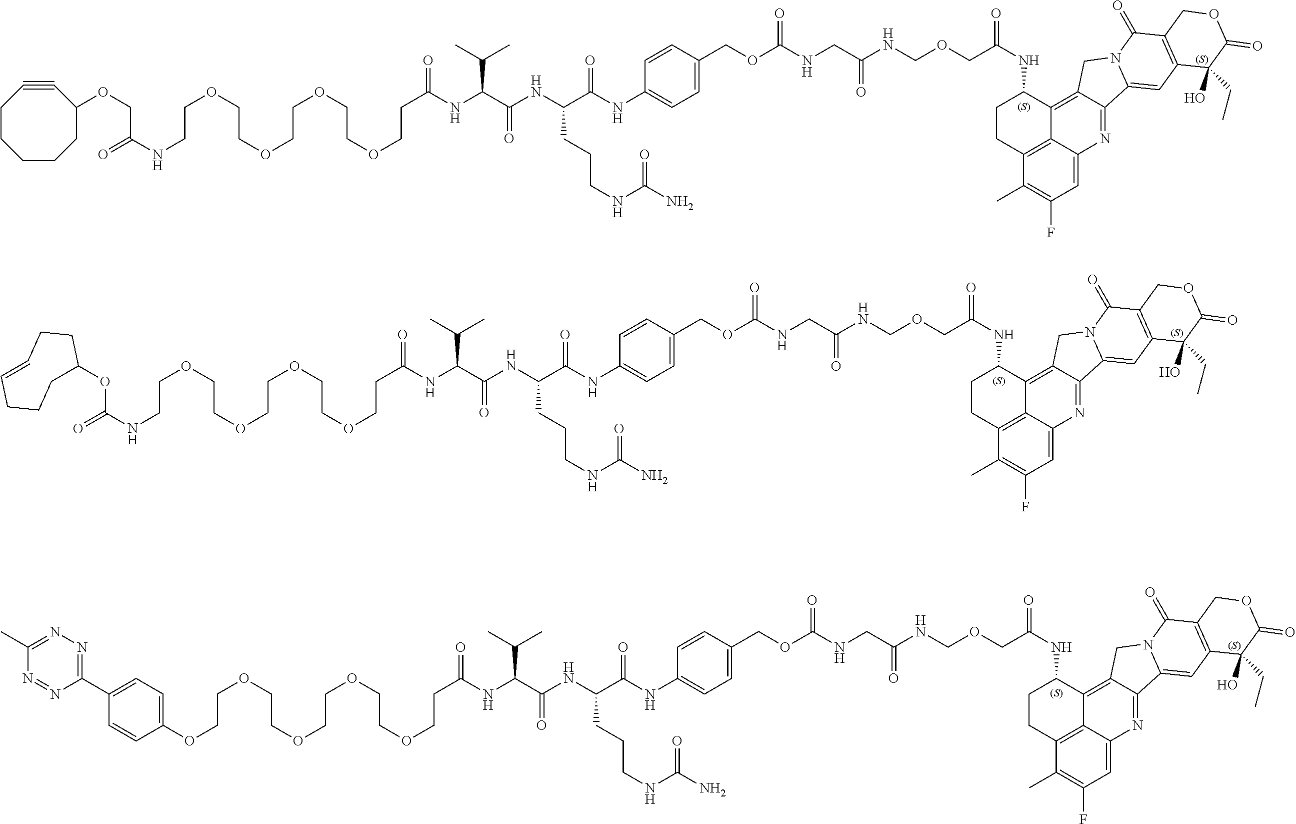

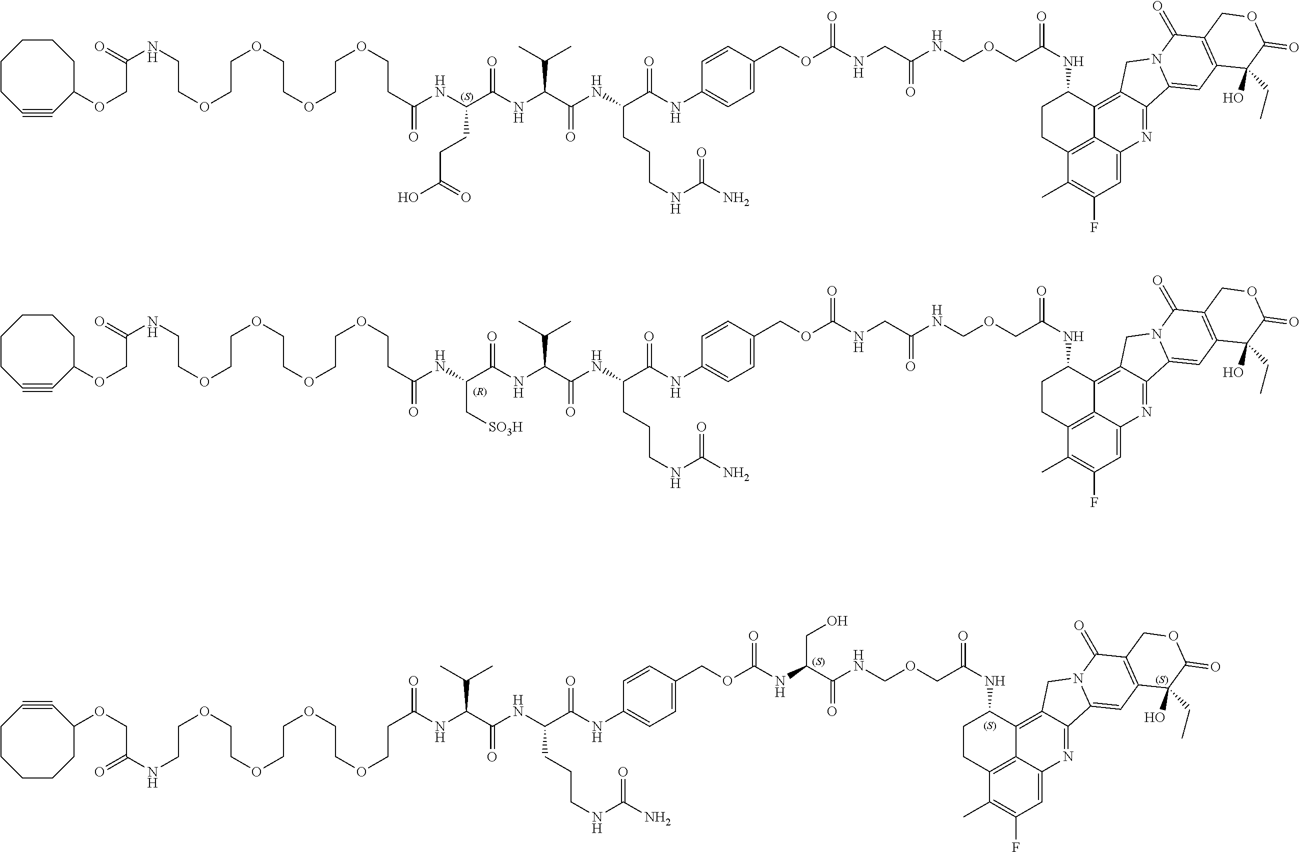

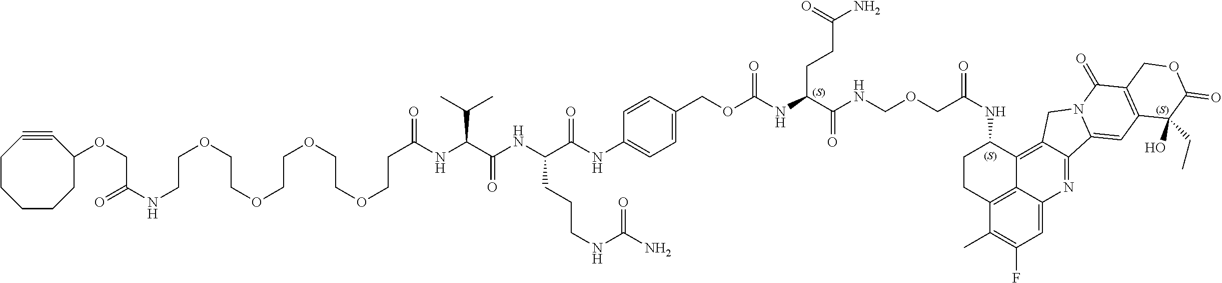

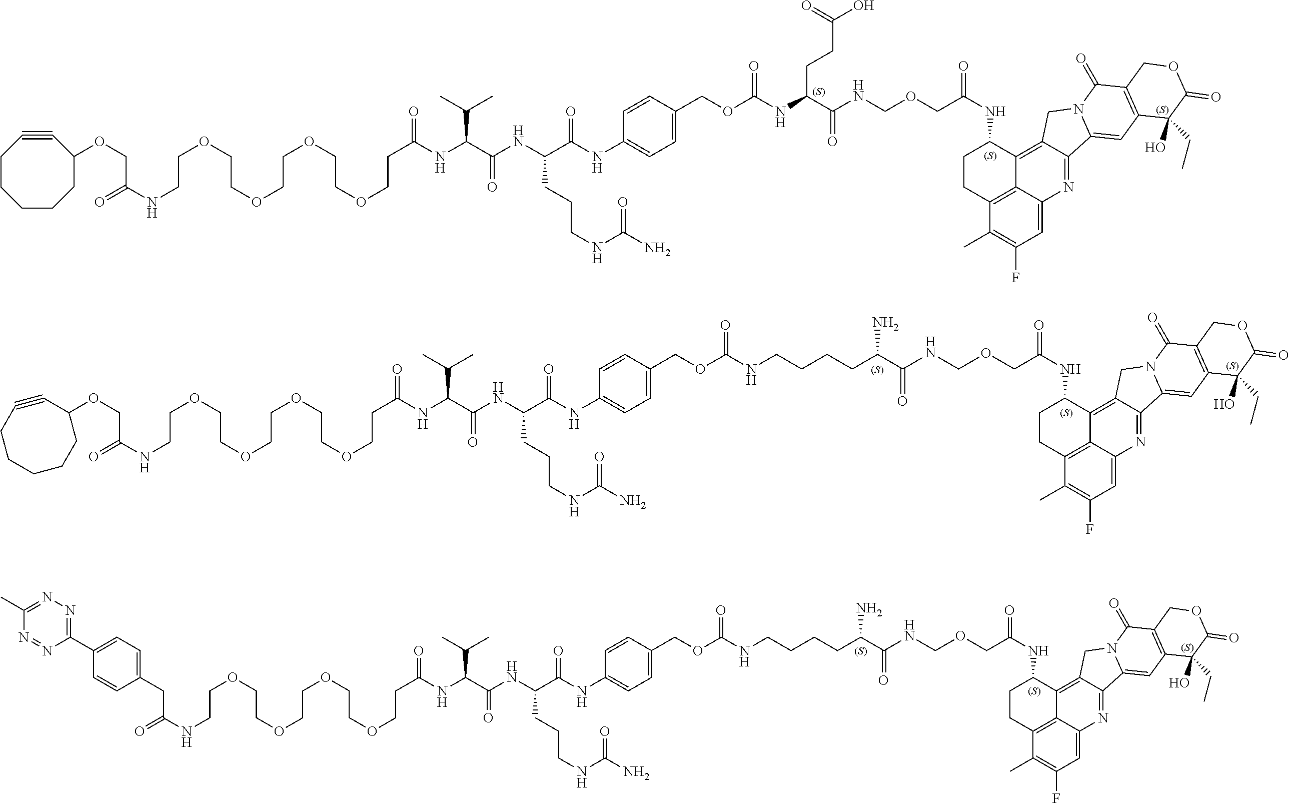

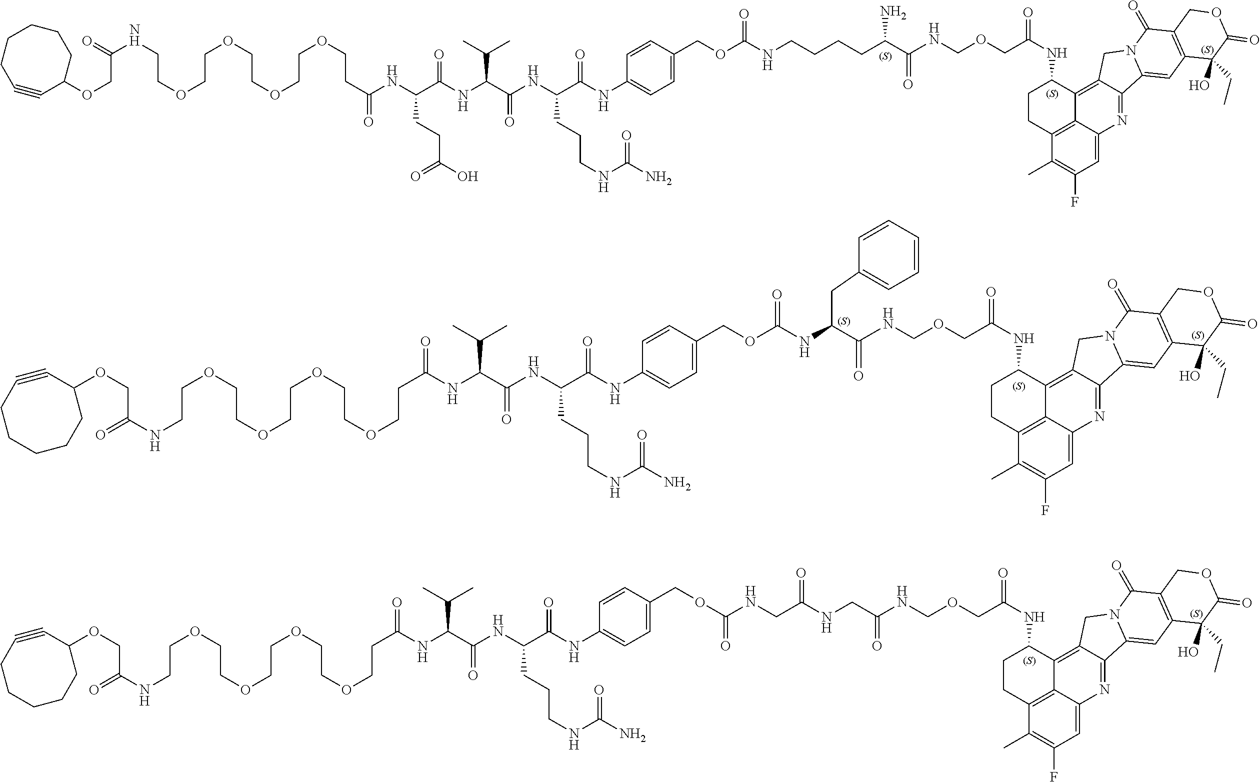

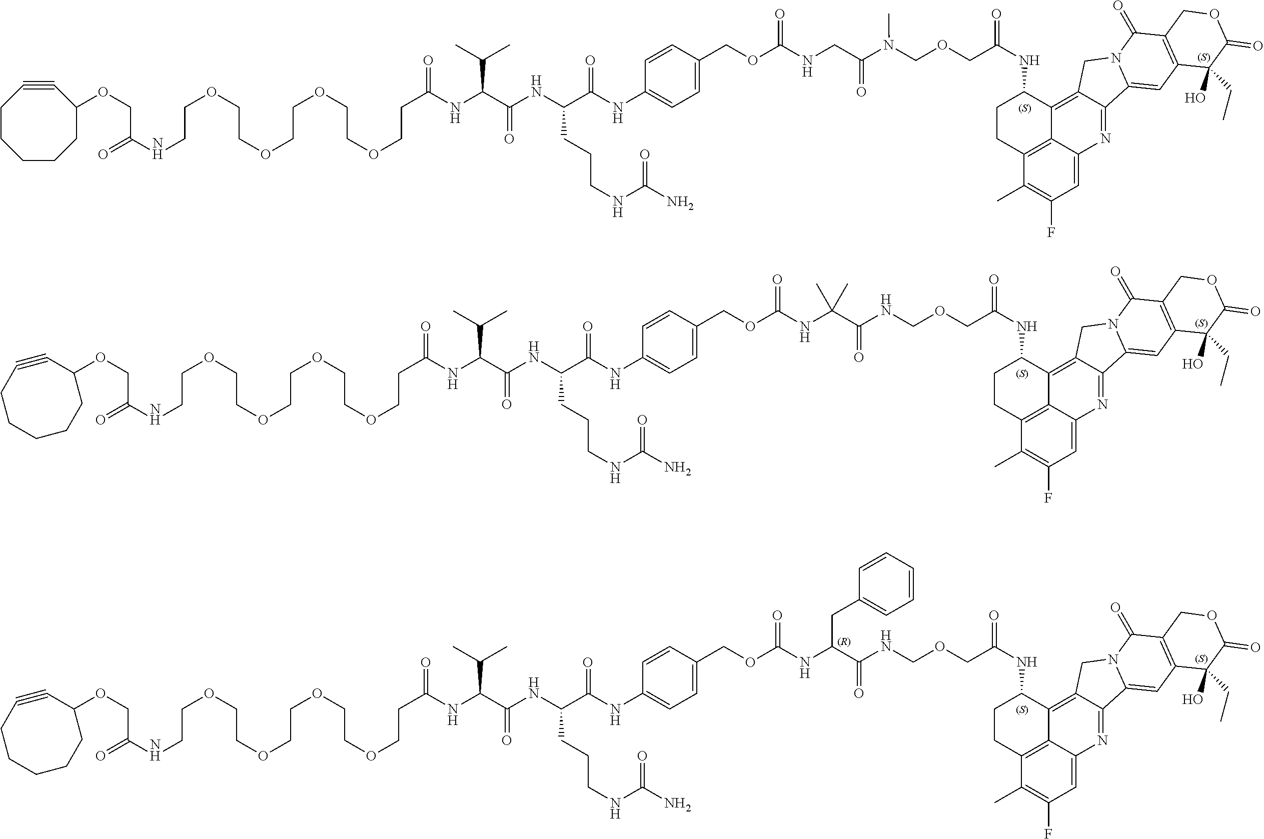

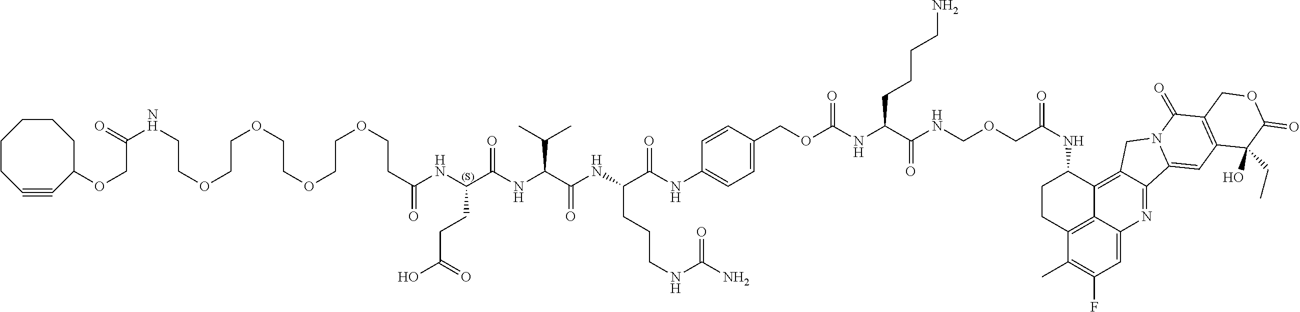

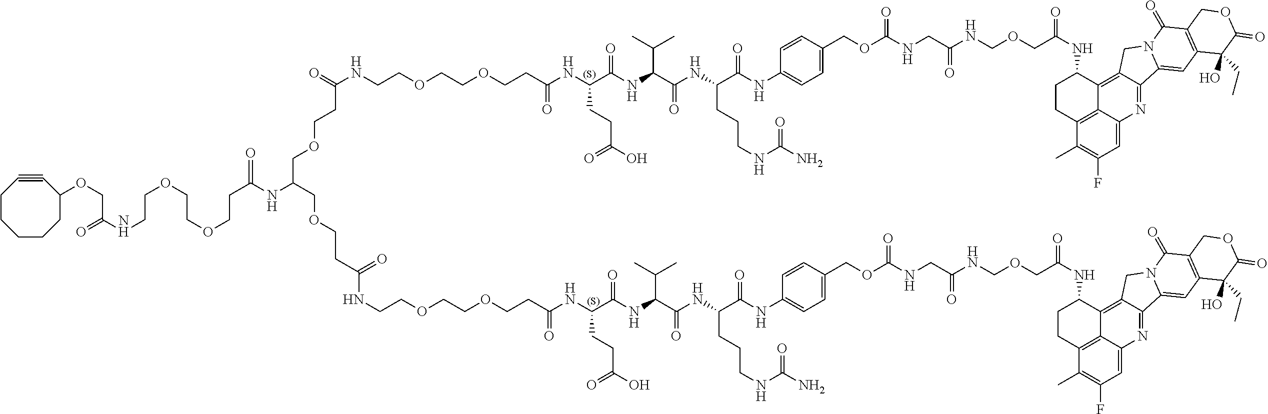

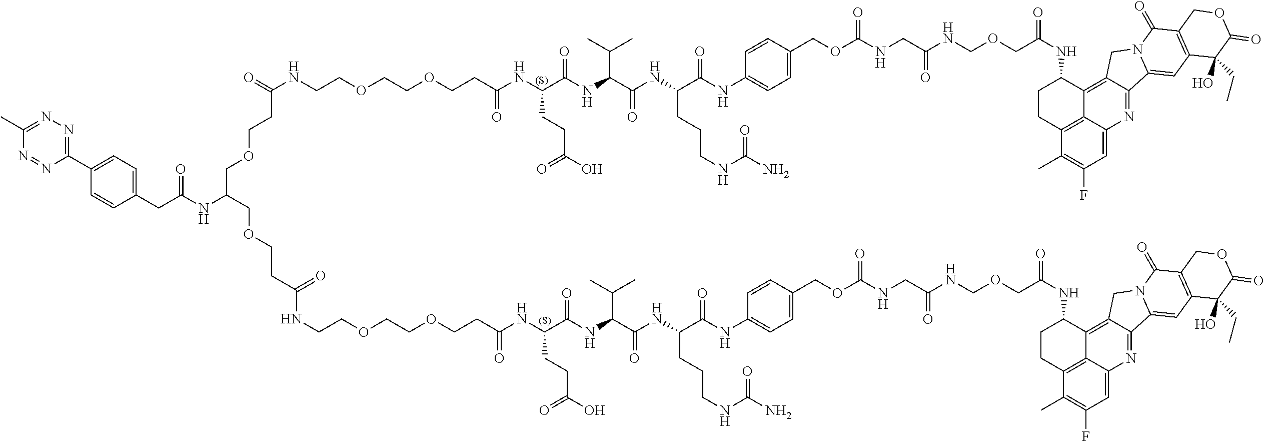

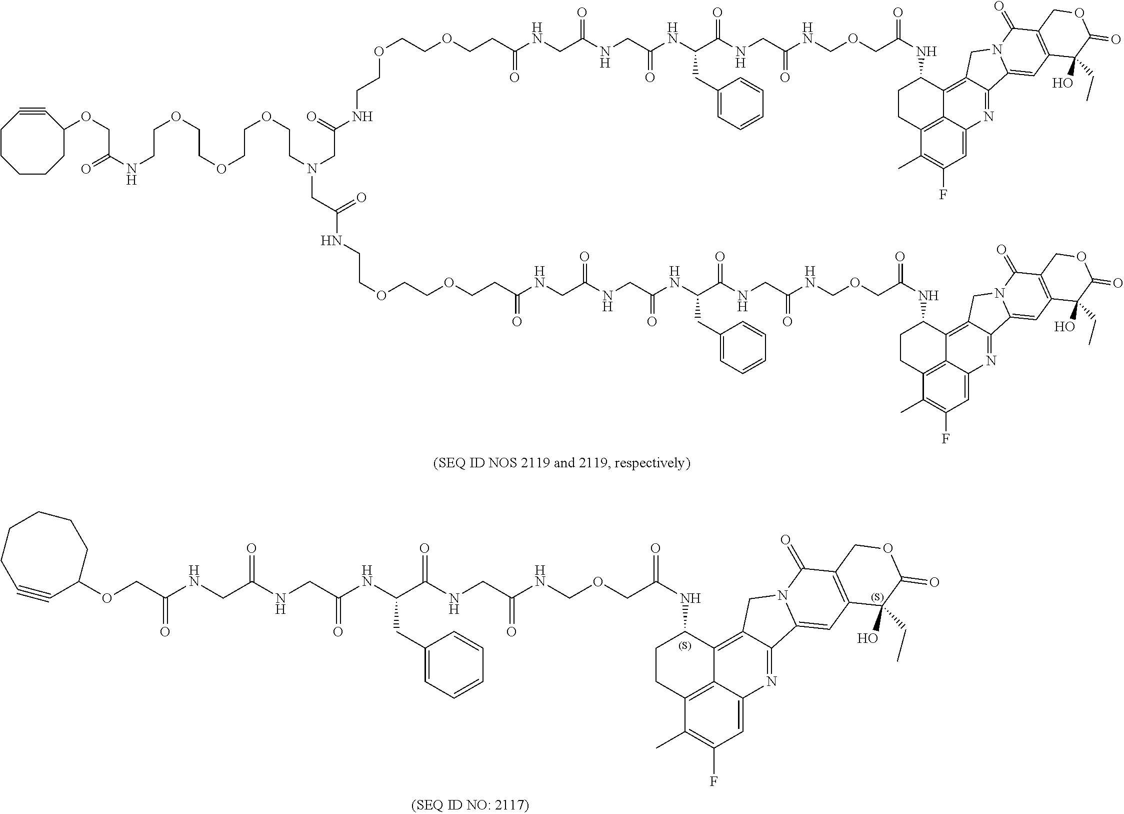

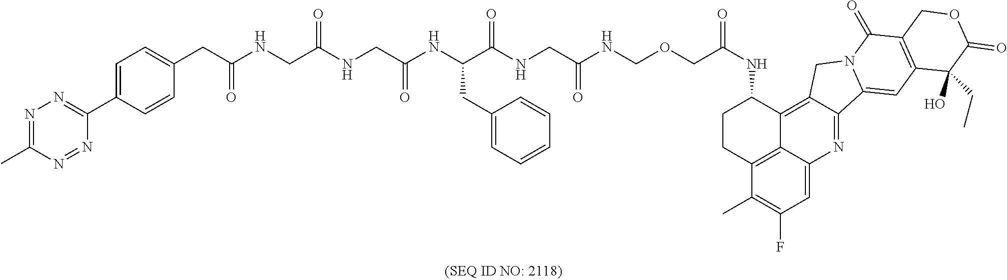 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)Cc1ccc(-c2nnc(C)nn2)cc1)CC3]

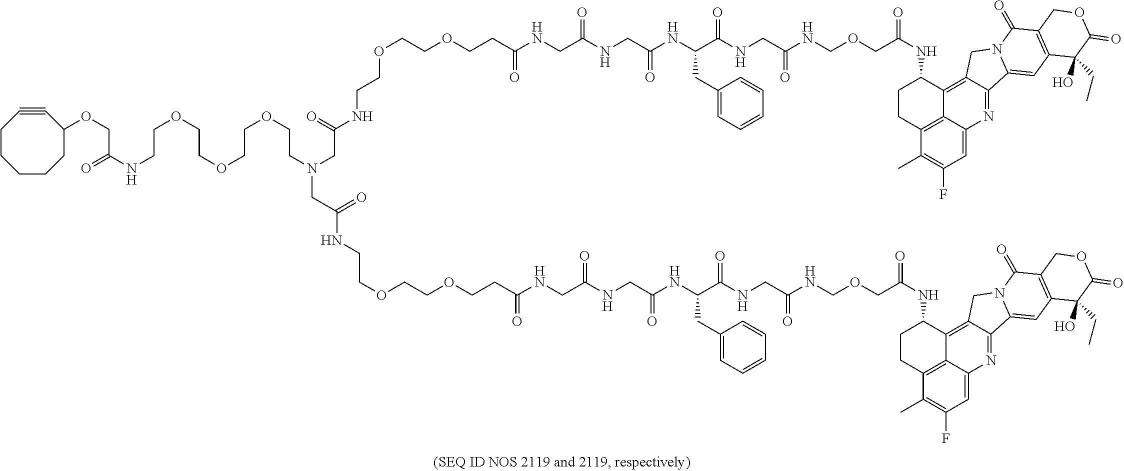 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCOCCOCCNC(=O)CN(CCOCCOCCOCCNC(=O)COC1C#CCCCCC1)CC(=O)NCCOCCOCCC(=O)NCC(=O)NCC(=O)N[C@@H](Cc1ccccc1)C(=O)NCC(=O)NCOCC(=O)N[C@H]1CCc2c(C)c(F)cc4nc5c(c1c24)Cn1c-5cc2c(c1=O)COC(=O)[C@]2(O)CC)CC3]

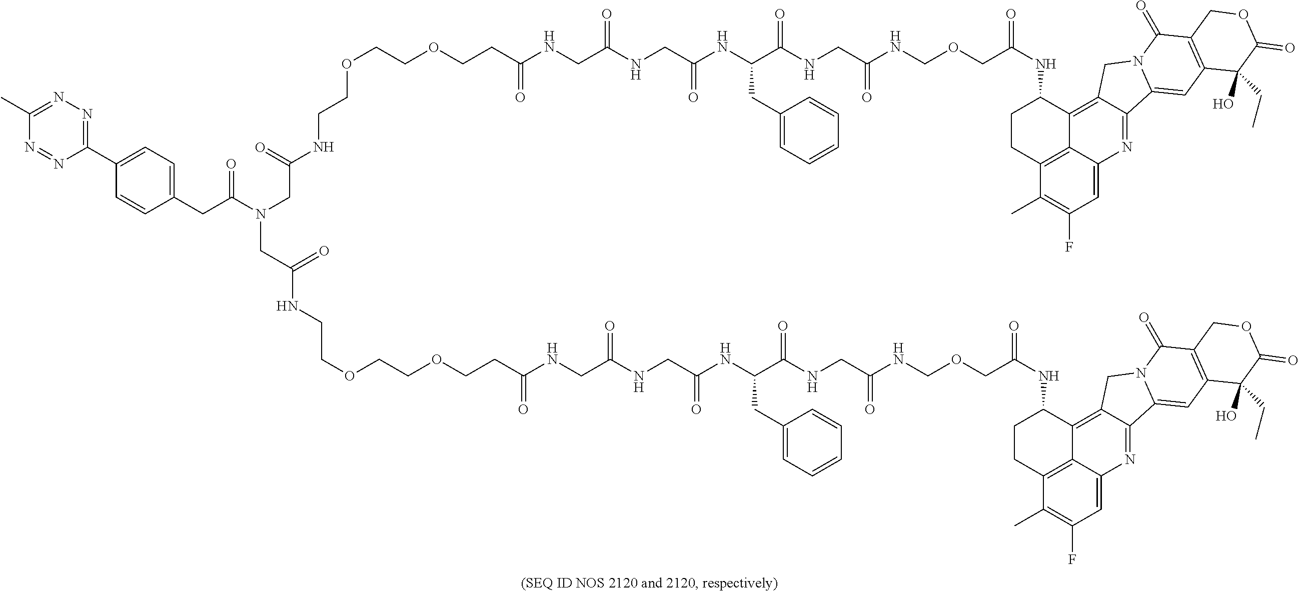 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCOCCOCCNC(=O)CN(CC(=O)NCCOCCOCCC(=O)NCC(=O)NCC(=O)N[C@@H](Cc1ccccc1)C(=O)NCC(=O)NCOCC(=O)N[C@H]1CCc2c(C)c(F)cc4nc5c(c1c24)Cn1c-5cc2c(c1=O)COC(=O)[C@]2(O)CC)C(=O)Cc1ccc(-c2nnc(C)nn2)cc1)CC3]

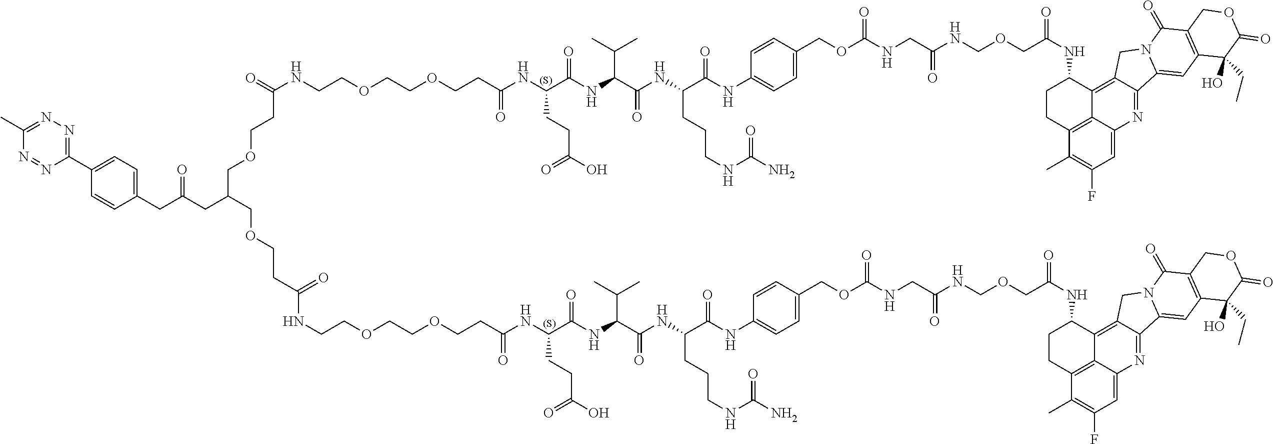

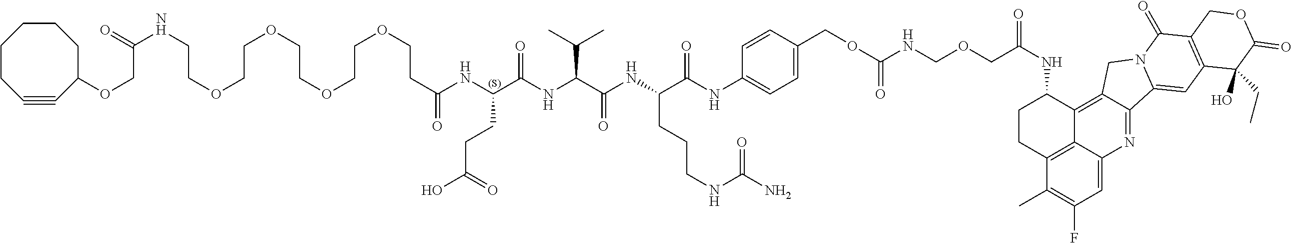

In one embodiment, the structure is selected from the group consisting of:

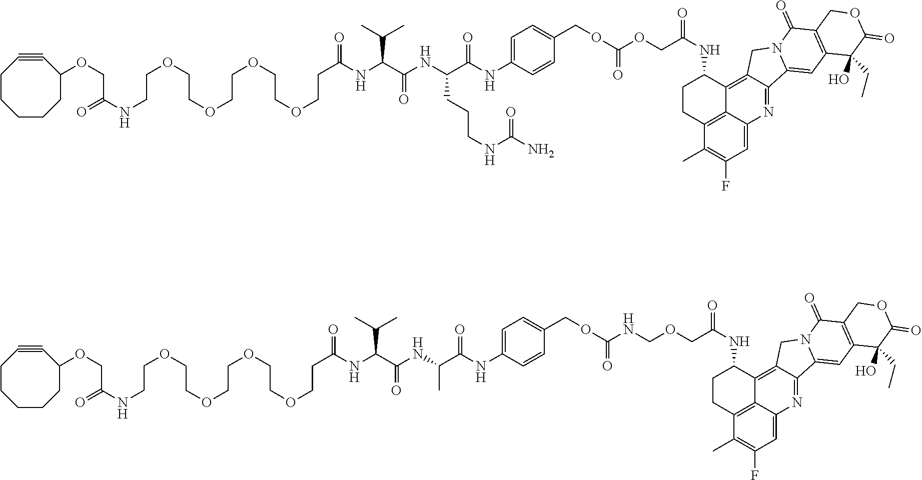 [SMILES: CC[C@@]1(O)C(=O)OCc2c1cc1n(c2=O)Cc2c-1nc1cc(F)c(C)c3c1c2[C@@H](NC(=O)COC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3 ; CC[C@@]1(O)C(=O)OCc2c1cc1n(c2=O)Cc2c-1nc1cc(F)c(C)c3c1c2[C@@H](NC(=O)COCNC(=O)OCc1ccc(NC(=O)[C@H](C)NC(=O)[C@@H](NC(=O)CCOCCOCCOCCOCCNC(=O)COC2C#CCCCCC2)C(C)C)cc1)CC3]

These and other aspects of the present disclosure will become apparent to those skilled in the art after a reading of the following detailed description of the disclosure, including the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic demonstrating two-step site-specific generation of Dxd-ADCs according to an embodiment of the disclosure. The first step is conjugation of one or more first linkers (L1-B′) with a glutamine residue on an antibody via a transglutaminase (e.g., MTG)-mediated conjugation reaction. The second step is conjugation of antibody-L1-B to one or more Linker 2-Payloads (L2P).

FIGS. 2A and 2B are schematics demonstrating specific non-limiting embodiments of the disclosure. FIG. 2A is a schematic of a two-step site-specific generation of Dxd-ADCs with glutamine residues at position 295 having a DAR of 2 times n times m according to an embodiment of the present disclosure. FIG. 2B is a schematic of a two-step site-specific generation of Dxd-ADCs with a glutamine residue at positions 295 and 297 having a DAR of 4 times n times m according to an embodiment of the present disclosure.

FIG. 3A is a schematic demonstrating two-step site-specific generation of one specific embodiment of a Dxd-ADC according to the disclosure. The first step is to conjugate a linear first linker 1 (L1-B′) comprising one azide moiety (—N₃) to glutamine residues at positions 295 and 297 of an antibody via an MTG-mediated conjugation reaction, generating an antibody having 4 azide-comprising linkers attached to it (Ab-(N₃)₄). The second step is to attach Ab-(N₃)₄ to a specific Linker2-Payload (L2P) via the azide-cycloalkyne 1,3 cycloaddition reaction, generating a Dxd-ADC with a DAR of 4. FIG. 3B depicts schematics of ADCs and exemplary amino azido linkers having a DAR of 2 or 4 suitable for use in an embodiment of the present disclosure depicted in FIG. 3A.

FIG. 4A is a schematic demonstrating two-step site-specific generation of one specific embodiment of a Dxd-ADC according to the disclosure. The first step is to conjugate a branched first linker 1 (L1-B′) comprising two azide moieties (—N₃) to glutamine residues at positions 295 and 297 of an antibody via an MTG-mediated conjugation reaction, generating an antibody having 8 azide-comprising linkers attached to it (Ab-(N₃)₈). The second step is to attach Ab-(N₃)₈ to a specific Linker2-Payload (L2P) via the azide-cycloalkyne 1,3 cycloaddition reaction, generating a Dxd-ADC with a DAR of 8. FIG. 4B depicts schematics of ADCs and exemplary branched alkyl azide amine linkers suitable for use in an embodiment of the present disclosure depicted in FIG. 4A.

FIG. 5 is a schematic of 2-step antibody-drug conjugation according to an embodiment of the present disclosure. Step 1: site-specific conjugation of Handle-functionalized amine with an Antibody generated a drug conjugate containing 2, 4 or 8 handles per antibody. Here, AL=non-branched Handle-functionalized amine, BL=branched Handle-functionalized amine. Step 2: click reactions between Handle-functionalized antibodies and a Linker-Payload (LP) to generate the site-specific ADCs.

FIG. 6 depicts an exemplary conjugation procedure according to the present disclosure.

FIG. 7A depicts three approaches to the preparation of antibody-drug conjugates according to the disclosure. For approaches 1 and 2, the handle may be bivalent or multivalent. An amine handle can be conjugated to an antibody via transglutaminase-mediated conjugation to generate an Ab-Handle; another moiety in the handle of the Ab-Handle can be clicked with a linker-payload to generate an ADC. Where the handle has a diene, the linker-payload has a dienophile, or vice versa. For approach 3, shown in FIG. 7B, the linker-payload may be conjugated to an antibody directly; LL containing an amine moiety that can be conjugated with an antibody via transglutaminase-mediated conjugation; LL containing a moiety reacting with cysteine-SH can be conjugated to antibody-cystine via Michael addition.

FIG. 8 is a graph showing Linker-ProDXd LP1 in mouse whole blood (SEQ ID NO: 2121).

FIG. 9 shows the schematic process of the preparation of the liver S9 and the liver microsomes from hepatocytes.

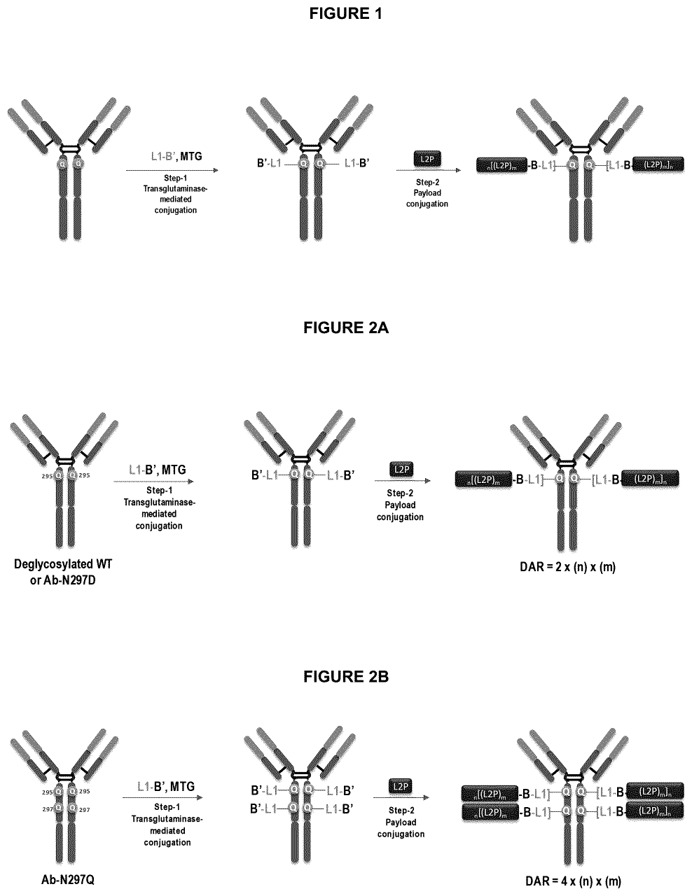

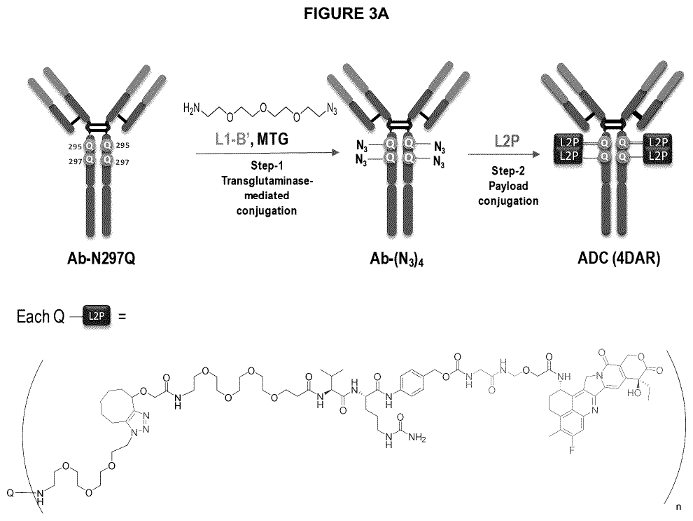

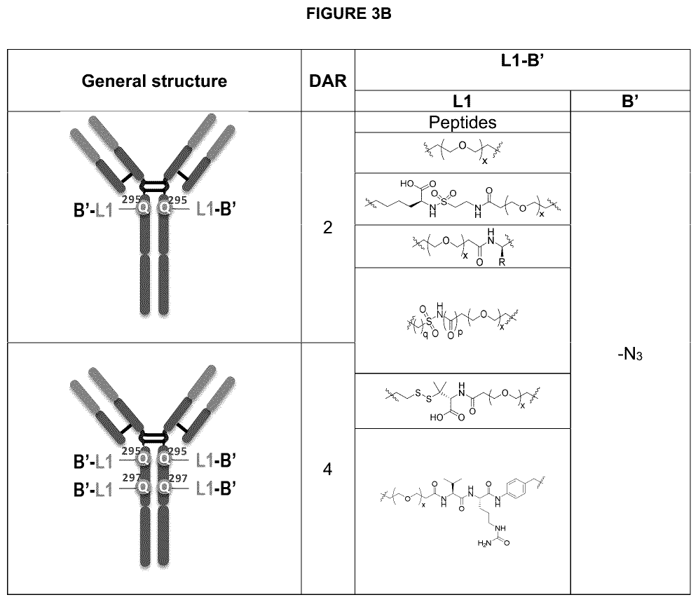

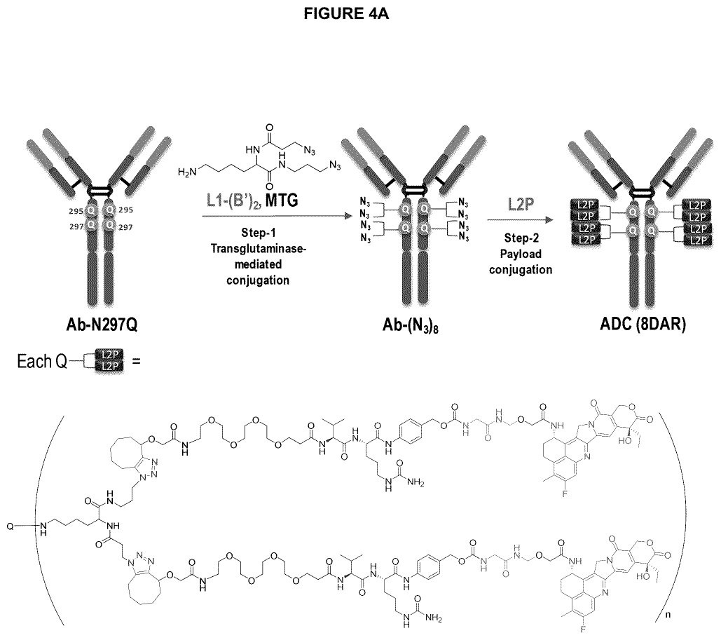

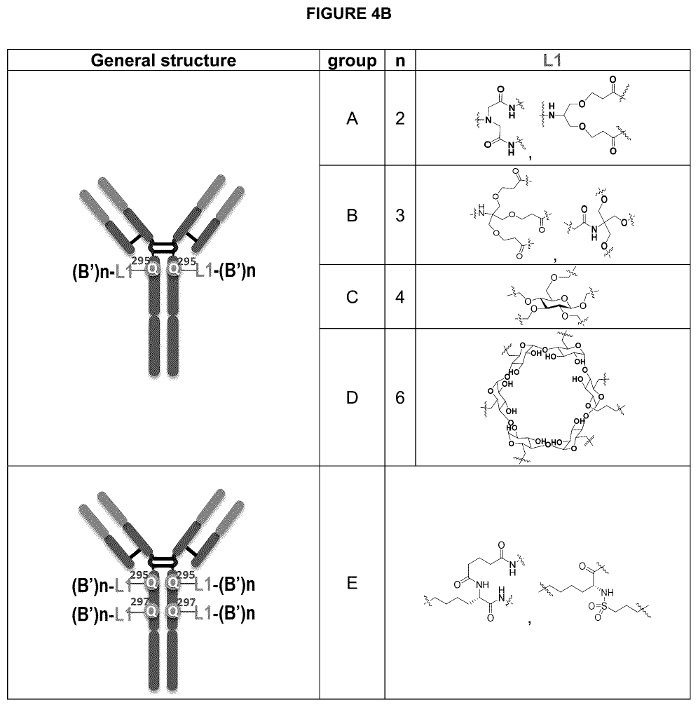

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive. Therefore, specific structural and functional details disclosed herein are not to be interpreted as limiting, but merely as a representative basis for teaching one skilled in the art to variously employ the present disclosure.

### Definitions

Unless defined otherwise, all technical and scientific terms used herein have the same meaning as commonly understood by one of ordinary skill in the art to which this disclosure belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician. In some embodiments, treatment comprises methods wherein cells are ablated in such manner where disease is indirectly impacted. In certain embodiments, treatment comprises depleting immune cells as a hematopoietic conditioning regimen prior to therapy.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable salt”, as used in connection with compositions of the disclosure, refers to any salt suitable for administration to a patient. Suitable salts include, but are not limited to, those disclosed in Berge et al., “Pharmaceutical Salts”, J. Pharm. Sci., 1977, 66:1, incorporated herein by reference. Examples of salts include, but are not limited to, acid derived, base derived, organic, inorganic, amine, and alkali or alkaline earth metal salts, including but not limited to calcium salts, magnesium salts, potassium salts, sodium salts, salts of hydrochloric acid, hydrobromic acid, sulfuric acid, nitric acid, phosphoric acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methane sulfonic acid, ethane sulfonic acid, p toluene sulfonic acid, salicylic acid, and the like. In some examples, a payload described herein (e.g., a rifamycin analog described herein) comprises a tertiary amine, where the nitrogen atom in the tertiary amine is the atom through which the payload is bonded to a linker or a linker-spacer. In such instances, bonding to the tertiary amine of the payload yields a quaternary amine in the linker-payload molecule. The positive charge on the quaternary amine can be balanced by a counter ion (e.g., chloro, bromo, iodo, or any other suitably charged moiety such as those described herein).

Ranges can be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 1-20 carbon atoms in its backbone (e.g., C₁-C₂₀ for straight chain, C₂-C₂₀ for branched chain), and alternatively, about 1-10 carbon atoms, or about 1 to 6 carbon atoms. In some embodiments, a cycloalkyl ring has from about 3-10 carbon atoms in their ring structure where such rings are monocyclic or bicyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

As used herein, the term “alkenyl” refers to an alkyl group, as defined herein, having one or more double bonds.

As used herein, the term “alkynyl” refers to an alkyl group, as defined herein, having one or more triple bonds.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, binaphthyl, anthracyi and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring.

The term “halogen” means F, Cl, Br, or I; the term “halide” refers to a halogen radical or substituent, namely —F, —Cl, —Br, or —I.

The term “click chemistry” refers to a class of biocompatible small molecule reactions commonly used in bioconjugation, allowing the joining of substrates of choice with specific biomolecules. Click chemistry is not a single specific reaction, but describes a way of generating products that follow examples in nature, which also generates substances by joining small modular units. Click chemistry is not limited to biological conditions: the concept of a “click” reaction may be used in chemoproteomic, pharmacological, and various biomimetic applications. Specific non-limiting examples of click chemistry reactions include:
(a) strain-promoted azide/dibenzocyclooctyne-amine (DBCO) click chemistry;
(b) inverse electron demand Diels-Alder (IED-DA) tetrazine/trans-cyclooctene (TCO) click chemistry;
(c) inverse electron demand Diels-Alder (IED-DA) tetrazine/norbonene click chemistry;
(d) Diels-Alder maleimide/furan click-chemistry;
(e) Staudinger ligation; and
(f) nitrile-oxide/norbonene cycloaddition click chemistry.

The term “adduct”, e.g., “an adduct of group B” or “a click chemistry adduct” of the present disclosure encompasses any moiety comprising the product of an addition reaction, e.g., an addition reaction of group B or a click chemistry addition reaction, independent of the synthetic steps taken to produce the moiety.

The term “covalent attachment” means formation of a covalent bond, i.e., a chemical bond that involves sharing of one or more electron pairs between two atoms. Covalent bonding may include different interactions, including but not limited to σ-bonding, τ-bonding, metal-to-metal bonding, agostic interactions, bent bonds, and three-center two-electron bonds. When a first group is said to be “capable of covalently attaching” to a second group, this means that the first group is capable of forming a covalent bond with the second group, directly or indirectly, e.g., through the use of a catalyst or under specific reaction conditions. Non-limiting examples of groups capable of covalently attaching to each other may include, e.g., an amine and a carboxylic acid (forming an amide bond), a diene and a dienophile (via a Diels-Alder reaction), and an azide and an alkyne (forming a triazole via a 1,3-cycloaddition reaction).

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the disclosure.

Unless otherwise stated, all tautomeric forms of the compounds of the disclosure are within the scope of the disclosure.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹¹C- or ¹³C- or ¹⁴C-enriched carbon are within the scope of this disclosure.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Unless otherwise stated, all crystalline forms of the compounds of the disclosure and salts thereof are also within the scope of the disclosure. The compounds of the disclosure may be isolated in various amorphous and crystalline forms, including without limitation forms which are anhydrous, hydrated, non-solvated, or solvated. Example hydrates include hemihydrates, monohydrates, dihydrates, and the like. In some embodiments, the compounds of the disclosure are anhydrous and non-solvated. By “anhydrous” is meant that the crystalline form of the compound contains essentially no bound water in the crystal lattice structure, i.e., the compound does not form a crystalline hydrate.

As used herein, “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content. The different crystalline lattices can be identified by solid state characterization methods such as by X-ray powder diffraction (PXRD). Other characterization methods such as differential scanning calorimetry (DSC), thermogravimetric analysis (TGA), dynamic vapor sorption (DVS), solid state NMR, and the like further help identify the crystalline form as well as help determine stability and solvent/water content.

Crystalline forms of a substance include both solvated (e.g., hydrated) and non-solvated (e.g., anhydrous) forms. A hydrated form is a crystalline form that includes water in the crystalline lattice. Hydrated forms can be stoichiometric hydrates, where the water is present in the lattice in a certain water/molecule ratio such as for hemihydrates, monohydrates, dihydrates, etc. Hydrated forms can also be non-stoichiometric, where the water content is variable and dependent on external conditions such as humidity.

In some embodiments, the compounds of the disclosure are substantially isolated. By “substantially isolated” is meant that a particular compound is at least partially isolated from impurities. For example, in some embodiments a compound of the disclosure comprises less than about 50%, less than about 40%, less than about 30%, less than about 20%, less than about 15%, less than about 10%, less than about 5%, less than about 2.5%, less than about 1%, or less than about 0.5% of impurities. Impurities generally include anything that is not the substantially isolated compound including, for example, other crystalline forms and other substances.

Certain groups, moieties, substituents, and atoms are depicted with a wavy line. The wavy line can intersect or cap a bond or bonds. The wavy line indicates the atom through which the groups, moieties, substituents, or atoms are bonded. For example, a phenyl group that

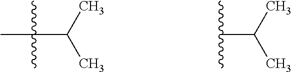

is substituted with a propyl group depicted as: HCH has the following structure:

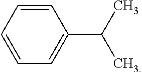

The expression “HER2” or “human epidermal growth factor receptor 2” refers to a member of the human epidermal growth factor receptor family. The protein is also known as NEU; NGL; HER2; TKR1; CD340; HER-2; MLN 19; HER-2/neu. HER2 can refer to the amino acid sequence as set forth in NCBI accession No. NP_004439.2. Amplification or over-expression of this oncogene has been shown to play an important role in the development and progression of certain aggressive types of breast cancer. In recent years the protein has become an important biomarker and target of therapy for approximately 30% of breast cancer patient. All references to proteins, polypeptides and protein fragments herein are intended to refer to the human version of the respective protein, polypeptide or protein fragment unless explicitly specified as being from a non-human species. Thus, the expression “HER2” means human HER2 unless specified as being from a non-human species, e.g., “mouse HER2,” “monkey HER2,” etc.

The phrase “an antibody that binds HER2” or an “anti-HER2 antibody” includes antibodies and antigen-binding fragments thereof that specifically recognize HER2.

The phrase an “anti-HER2/HER2” antibody, e.g., an “anti-HER2/HER2 bispecific antibody” includes antibodies and antigen-binding fragments thereof that specifically recognize two different HER2 epitopes. In some embodiments, bispecific antibodies and antigen-binding fragments thereof comprise a first antigen-binding domain (D1) which specifically binds a first epitope of human HER2 and a second antigen-binding domain (D2) which specifically binds a second epitope of human HER2.

The expression “STEAP2,” as used herein, refers to six-transmembrane epithelial antigen of prostate 2. STEAP2 is an integral, six-transmembrane-spanning protein that is highly expressed in prostate epithelial cells and is a cell-surface marker for prostate cancer, for example STEAP2 was found to be expressed in significant levels on an LNCaP prostate cell line (Porkka, et al. Lab Invest 2002, 82:1573-1582). STEAP2 (UniProtKB/Swiss-Prot: Q8NFT2.3) is a 490-amino acid protein encoded by STEAP2 gene located at the chromosomal region 7q21 in humans, see e.g., the amino acid sequence of human STEAP2 as set forth in Tables 5 and 6.

As used herein, “an antibody that binds STEAP2” or an “anti-STEAP2 antibody” includes antibodies and antigen-binding fragments thereof that specifically recognize STEAP2.

The phrase “an antibody that binds MET” or an “anti-MET antibody” includes antibodies and antigen-binding fragments thereof that specifically recognize MET. The expressions “MET,” “c-Met,” and the like, as used herein, refer to the human membrane spanning receptor tyrosine kinase.

The phrase an “anti-MET/MET” antibody, e.g., an “anti-MET/MET bispecific antibody” includes antibodies and antigen-binding fragments thereof that specifically recognize two different MET epitopes. In some embodiments, bispecific antibodies and antigen-binding fragments thereof comprise a first antigen-binding domain (D1) which specifically binds a first epitope of human MET and a second antigen-binding domain (D2) which specifically binds a second epitope of human MET.

All amino acid abbreviations used in this disclosure are those accepted by the United States Patent and Trademark Office as set forth in 37 C.F.R. § 1.822 (B)(J).

The term “protein” means any amino acid polymer having more than about 20 amino acids covalently linked via amide bonds. As used herein, “protein” includes biotherapeutic proteins, recombinant proteins used in research or therapy, trap proteins and other Fc-fusion proteins, chimeric proteins, antibodies, monoclonal antibodies, human antibodies, bispecific antibodies, antibody fragments, nanobodies, recombinant antibody chimeras, scFv fusion proteins, cytokines, chemokines, peptide hormones, and the like. Proteins can be produced using recombinant cell-based production systems, such as the insect bacculovirus system, yeast systems (e.g., Pichia sp.), mammalian systems (e.g., CHO cells and CHO derivatives like CHO-K1 cells).

The term “natural amino acid” and “natural amino acid side chain” means any naturally occurring amino acid, and side chain thereof, respectively. These include 20 L-amino acids naturally occurring in the human body.

The term “nonnatural (also spelled non-natural and non natural) amino acid” and “nonnatural amino acid side chain” means an amino acid, and side chain thereof, respectively, which does not naturally occur in the subject organism, e.g., a human. Such nonnatural amino acids may be produced synthetically or generated naturally in a different setting, e.g., in a different organism. Non-limiting examples of nonnatural amino acids may include D-amino acids, homo-amino acids, beta-homo-amino acids, N-methyl amino acids, α-methyl amino acids, and amino acids that occur in, e.g., microbial peptides, such as citrulline (Cit), hydroxyproline (Hyp), norleucine (Nle), 3-nitrotyrosine, nitroarginine, ornithine (Orn), naphtylalanine (Nal), Abu, DAB, methionine sulfoxide or methionine sulfone.

All references to proteins, polypeptides and protein fragments herein are intended to refer to the human version of the respective protein, polypeptide or protein fragment unless explicitly specified as being from a non-human species. Thus, the expression “STEAP2” means human STEAP2 unless specified as being from a non-human species, e.g., “mouse STEAP2,” “monkey STEAP2,” etc.

The amino acid sequence of an antibody can be numbered using any known numbering schemes, including those described by Kabat et al., (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme). Unless otherwise specified, the numbering scheme used herein is the Kabat numbering scheme. However, selection of a numbering scheme is not intended to imply differences in sequences where they do not exist, and one of skill in the art can readily confirm a sequence position by examining the amino acid sequence of one or more antibodies. Unless stated otherwise, the “EU numbering scheme” is generally used when referring to a residue in an antibody heavy chain constant region (e.g., as reported in Kabat et al., supra).

The term “glutaminyl-modified antibody” refers to an antibody with at least one covalent linkage from a glutamine side chain to a primary amine compound of the present disclosure. In particular embodiments, the primary amine compound is linked through an amide linkage on the glutamine side chain. In certain embodiments, the glutamine is an endogenous glutamine. In other embodiments, the glutamine is an endogenous glutamine made reactive by polypeptide engineering (e.g., via amino acid deletion, insertion, substitution, or mutation on the polypeptide). In additional embodiments, the glutamine is polypeptide engineered with an acyl donor glutamine-containing tag (e.g., glutamine-containing peptide tags, Q-tags or TGase recognition tag).

The term “TGase recognition tag” refers to a sequence of amino acids comprising an acceptor glutamine residue and that when incorporated into (e.g., appended to) a polypeptide sequence, under suitable conditions, is recognized by a TGase and leads to cross-linking by the TGase through a reaction between an amino acid side chain within the sequence of amino acids and a reaction partner. The recognition tag may be a peptide sequence that is not naturally present in the polypeptide comprising the TGase recognition tag. In some embodiments, the TGase recognition tag comprises at least one Gln. In some embodiments, the TGase recognition tag comprises an amino acid sequence XXQX, wherein X is any amino acid (e.g., conventional amino acid Leu, Ala, Gly, Ser, Val, Phe, Tyr, His, Arg, Asn, Glu, Asp, Cys, Gln, lie, Met, Pro, Thr, Lys, or Trp or nonconventional amino acid). In some embodiments, the acyl donor glutamine-containing tag comprises an amino acid sequence selected from the group consisting of LLQGG (SEQ ID NO:1936), LLQG (SEQ ID NO:1937), LSLSQG (SEQ ID NO:1938), gGGLLQGG (SEQ ID NO:1939), gLLQG (SEQ ID NO:1940), LLQ, gSPLAQSHGG (SEQ ID NO:1941), gLLQGGG (SEQ ID NO:1942), gLLQGG (SEQ ID NO:1943), gLLQ (SEQ ID NO:1944), LLQLLQGA (SEQ ID NO:1945), LLQGA (SEQ ID NO:1946), LLQYQGA (SEQ ID NO:1947), LLQGSG (SEQ ID NO:1948), LLQYQG (SEQ ID NO:1949), LLQLLQG (SEQ ID NO:1950), SLLQG (SEQ ID NO:1951), LLQLQ (SEQ ID NO:1952), LLQLLQ (SEQ ID NO:1953), and LLQGR (SEQ ID NO:1954). See for example, WO2012059882, the entire contents of which are incorporated herein.

The term “antibody,” as used herein, means any antigen-binding molecule or molecular complex comprising at least one complementarity determining region (CDR) that specifically binds to or interacts with a particular antigen. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2, and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments, the FRs of the antibody (or antigen-binding portion thereof) can be identical to the human germline sequences, or can be naturally or artificially modified. An amino acid consensus sequence can be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody can be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA can be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain can be of any size or amino acid composition and will generally comprise at least one CDR which is adjacent to or in frame with one or more framework sequences. In antigen-binding fragments having a VH domain associated with a VL domain, the VH and VL domains can be situated relative to one another in any suitable arrangement. For example, the variable region can be dimeric and contain VH-VH, VH-VL or VL-VL dimers. Alternatively, the antigen-binding fragment of an antibody can contain a monomeric VH or VL domain.

In certain embodiments, an antigen-binding fragment of an antibody can contain at least one variable domain covalently linked to at least one constant domain. Non-limiting, exemplary configurations of variable and constant domains that can be found within an antigen-binding fragment of an antibody of the present description include: (i) VH-CH1; (ii) VH-CH2; (iii) VH-CH3; (iv) VH-CH1-CH2; (V) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed herein, the variable and constant domains can be either directly linked to one another or can be linked by a full or partial hinge or linker region. A hinge region can consist of at least 2 (e.g., 5, 10, 15, 20, 40, 60, or more) amino acids which result in a flexible or semi-flexible linkage between adjacent variable and/or constant domains in a single polypeptide molecule.

Moreover, an antigen-binding fragment of an antibody of the present description can comprise a homo-dimer or hetero-dimer (or other multimer) of any of the variable and constant domain configurations listed herein in non-covalent association with one another and/or with one or more monomeric VH or VL domain (e.g., by disulfide bond(s)).

As with full antibody molecules, antigen-binding fragments can be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, can be adapted for use in the context of an antigen-binding fragment of an antibody of the present description using routine techniques available in the art.

The antibodies of the present description can function through complement-dependent cytotoxicity (CDC) or antibody-dependent cell-mediated cytotoxicity (ADCC). “Complement-dependent cytotoxicity” (CDC) refers to lysis of antigen-expressing cells by an antibody of the description in the presence of complement. “Antibody-dependent cell-mediated cytotoxicity” (ADCC) refers to a cell-mediated reaction in which nonspecific cytotoxic cells that express Fc receptors (FcRs) (e.g., Natural Killer (NK) cells, neutrophils, and macrophages) recognize bound antibody on a target cell and thereby lead to lysis of the target cell. CDC and ADCC can be measured using assays that are well known and available in the art. (See, e.g., U.S. Pat. Nos. 5,500,362 and 5,821,337, and Clynes et al. (1998) Proc. Natl. Acad. Sci. (USA) 95:652-656). The constant region of an antibody is important in the ability of an antibody to fix complement and mediate cell-dependent cytotoxicity. Thus, the isotype of an antibody can be selected on the basis of whether it is desirable for the antibody to mediate cytotoxicity.

In certain embodiments, the antibodies of the description, e.g., anti-HER2 antibodies, or anti-HER2/HER2 bispecific antibodies, or anti-MET antibodies, or anti-MET/MET bispecific antibodies, or anti-STEAP2 antibodies, are human antibodies. The term “human antibody,” as used herein, is intended to include antibodies having variable and constant regions derived from human germline immunoglobulin sequences. The human antibodies of the description can include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies can, in some embodiments, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (See, e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification. The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30: 105) to levels typically observed using a human IgG1 hinge. The instant description encompasses antibodies having one or more mutations in the hinge, CH2 or CH3 region which can be desirable, for example, in production, to improve the yield of the desired antibody form.

The antibodies of the description can be isolated or purified antibodies. An “isolated antibody” or “purified antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present description. For example, an antibody that has been purified from at least one component of a reaction or reaction sequence, is a “purified antibody” or results from purifying the antibody. An isolated antibody also includes an antibody in situ within a recombinant cell. Isolated antibodies are antibodies that have been subjected to at least one purification or isolation step. According to certain embodiments, an isolated antibody or purified antibody can be substantially free of other cellular material and/or chemicals.

The antibodies disclosed herein can comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present description includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived).

Furthermore, the antibodies of the present description can contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, improved drug-to-antibody ratio (DAR) for antibody-drug conjugates, etc. Antibodies and antigen-binding fragments obtained in this general manner are encompassed within the present description.

The term “aglycosylated antibody” refers to an antibody that does not comprise a glycosylation sequence that might interfere with a transglutamination reaction, for instance an antibody that does not have saccharide group at N297 on one or more heavy chains. In particular embodiments, an antibody heavy chain has an N297 mutation. In other words, the antibody is mutated to no longer have an asparagine residue at position 297 according to the EU numbering system as disclosed by Kabat et al. In particular embodiments, an antibody heavy chain has an N297Q or an N297D mutation. Such an antibody can be prepared by site-directed mutagenesis to remove or disable a glycosylation sequence or by site-directed mutagenesis to insert a glutamine residue at site apart from any interfering glycosylation site or any other interfering structure. Such an antibody also can be isolated from natural or artificial sources. Aglycosylated antibodies also include antibodies comprising a T299 or S298P or other mutations, or combinations of mutations that result in a lack of glycosylation.

The term “deglycosylated antibody” refers to an antibody in which a saccharide group at is removed to facilitate transglutaminase-mediated conjugation. Saccharides include, but are not limited to, N-linked oligosaccharides. In some embodiments, deglycosylation is performed at residue N297. In some embodiments, removal of saccharide groups is accomplished enzymatically, included but not limited to via PNGase.

The term “epitope” refers to an antigenic determinant that interacts with a specific antigen binding site in the variable region of an antibody molecule known as a paratope. A single antigen can have more than one epitope. Thus, different antibodies can bind to different areas on an antigen and can have different biological effects. Epitopes can be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope can include moieties of saccharides, phosphoryl groups, or sulfonyl groups on the antigen.

The terms “conjugated protein” or “conjugated antibody” as used herein refers to a protein or an antibody covalently linked to one or more chemical moieties. The chemical moiety can include an amine compound of the present disclosure. Linkers (LL) and payloads (P) suitable for use with the present disclosure are described in detail herein. In particular embodiments, a conjugated antibody comprising a therapeutic moiety is an antibody-drug conjugate (ADC), also referred to as an antibody-payload conjugate, or an antibody-linker-payload conjugate.

The term “Drug-to-Antibody Ratio” or (DAR) is the average number of therapeutic moieties, e.g., drugs, conjugated to a binding agent of the present disclosure.

The term “Linker Antibody Ratio” or (LAR), also denoted as the lower case I in some embodiments, is the average number of reactive primary amine compounds conjugated to a binding agent of the present disclosure. Such binding agents, e.g., antibodies, can be conjugated with primary amine compounds comprising, e.g., a suitable azide or alkyne. The resulting binding agent, which is functionalized with an azide or an alkyne can subsequently react with a therapeutic moiety comprising the corresponding azide or alkyne via the 1,3-cycloaddition reaction.

The phrase “pharmaceutically acceptable amount” refers to an amount effective or sufficient in treating, reducing, alleviating, or modulating the effects or symptoms of at least one health problem in a subject in need thereof. For example, a pharmaceutically acceptable amount of an antibody or antibody-drug conjugate is an amount effective for modulating a biological target using the antibody or antibody-drug-conjugates provided herein. Suitable pharmaceutically acceptable amounts include, but are not limited to, from about 0.001% up to about 10%, and any amount in between, such as about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% of an antibody or antibody-drug-conjugate provided herein.

The phrase “reaction pH” refers to the pH of a reaction after all reaction components or reactants have been added.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or gap, as discussed below. A nucleic acid molecule having substantial identity to a reference nucleic acid molecule can, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs gAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity can be adjusted upwards to correct for the conservative nature of the substitution. Means for making this adjustment are well-known to those of skill in the art. See, e.g., Pearson (1994) Methods Mol. Biol. 24: 307-331. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. In some embodiments, conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al. (1992) Science 256: 1443-1445. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, gCG software contains programs such as gap and Bestfit which can be used with default parameters to determine sequence homology or sequence identity between closely related polypeptides, such as homologous polypeptides from different species of organisms or between a wild type protein and a mutein thereof. See, e.g., gCG Version 6.1. Polypeptide sequences also can be compared using FASTA using default or recommended parameters, a program in gCG Version 6.1. FASTA (e.g., FASTA2 and FASTA3) provides alignments and percent sequence identity of the regions of the best overlap between the query and search sequences (Pearson (2000) supra). Another particular algorithm when comparing a sequence of the description to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402.

### Protein-Drug Conjugate Compounds

According to the foregoing objective and others, the present disclosure provides protein-drug conjugate compounds, e.g., antibody-drug conjugate compounds, and precursors and intermediates thereof, pharmaceutical compositions, and methods for treating certain diseases in a subject in need of such treatment. According to the disclosure, the protein-drug conjugate compounds provided herein comprise a glutaminyl-modified binding agent conjugated with a primary amine compound linked to a therapeutic moiety, e.g., camptothecin analog moiety, as described herein. Also provided are specific and efficient methods for producing protein-drug conjugates, e.g., antibody-drug conjugates, utilizing a combination of transglutaminase and 1,3-cycloaddition techniques. According to the disclosure, the protein-drug conjugate compounds provided herein comprise prodrugs of topoisomerase I inhibitor, e.g., prodrugs of Dxd.

In one aspect, the present disclosure provides an antibody or an antigen-binding fragment thereof conjugated to a compound having Formula (I)

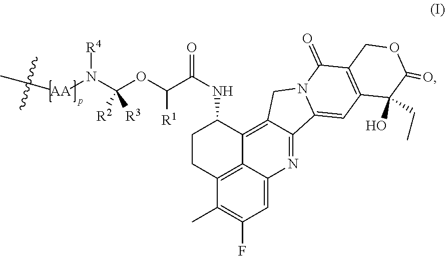

or a pharmaceutically acceptable salt thereof,
wherein R¹, R², R³, and R⁴ are independently hydrogen, a C₁₋₅ alkyl, or aryl;
AA is a natural or a nonnatural amino acid;
p is an integer from 1 to 6, and

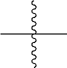

indicates the point of attachment to the antibody or the antigen-binding fragment thereof, directly or via a linker.

In one embodiment, the compound of Formula (I) is conjugated directly to the antibody or the antigen-binding fragment thereof.

In another embodiment, the compound of Formula (I) is conjugated to the antibody or the antigen-binding fragment thereof via a bivalent linker.

In one embodiment, p is 1. In another embodiment, p is 2, i.e., [AA]₂ is a peptide dimer of two amino acids. In another embodiment, p is 3. In another embodiment, p is 4. In another embodiment, p is 5. In another embodiment, p is 6. In any embodiment where p is greater than one, amino acids may be the same or different from each other. In one embodiment, p is 2 and the two amino acids are different from each other.

In one embodiment, p is 1 and the amino acid is a natural amino acid. In one embodiment, p is 1 and the natural amino acid is selected from glycine, alanine, valine, leucine, isoleucine, phenylalanine, tyrosine, threonine, lysine, asparagine, glutamine, aspartic acid, and glutamic acid. In one embodiment, p is 1 and the natural amino acid is selected from glycine, phenylalanine, threonine, lysine, glutamine, and glutamic acid.

In one embodiment, p is 1 and the amino acid is a nonnatural amino acid. In one embodiment, p is 1 and the nonnatural amino acid is selected from the group consisting of an R-amino acid, an N-methyl amino acid,

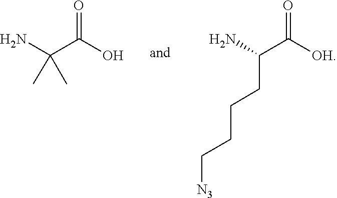

In another embodiment, p is 2, i.e., [AA]₂ is a peptide dimer of two amino acids. In one embodiment, p is 2 and both amino acids are glycines.

In one embodiment, R¹ is H.

In one embodiment, R² is H. In one embodiment, R³ is H. In one embodiment, R² and R³ are both Hs.

In one embodiment, R⁴ is H. In another embodiment, R⁴ is a C₁₋₅ alkyl. In one particular embodiment, R⁴ is a C₁ alkyl (a methyl).

In one embodiment, the compound of Formula (I) is referred to as a payload.

In one embodiment, the compound of Formula (I) comprises a compound selected from the group consisting of:

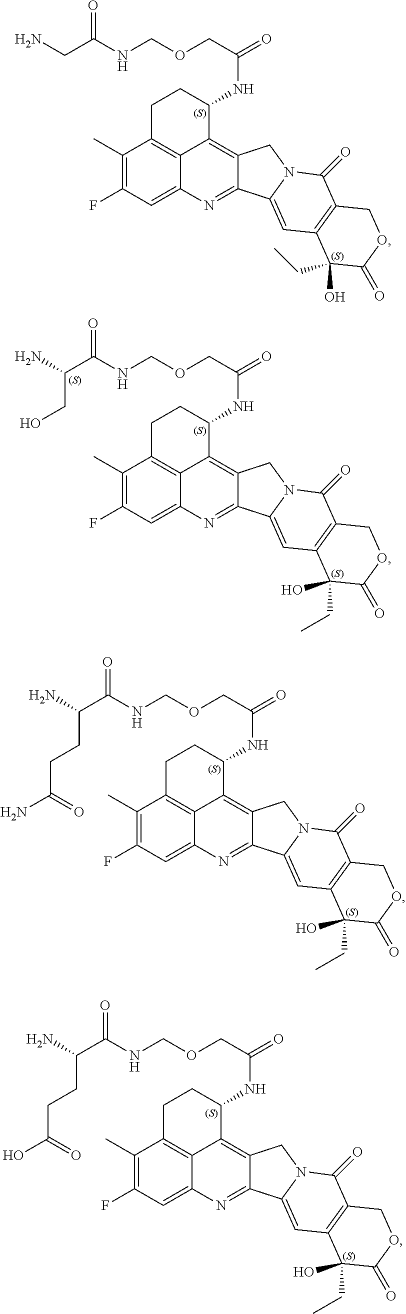

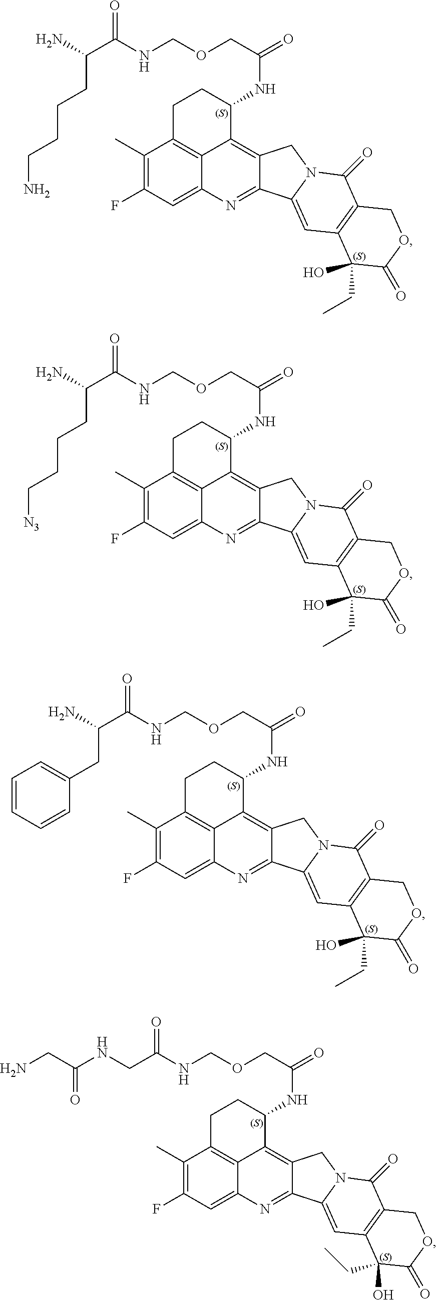

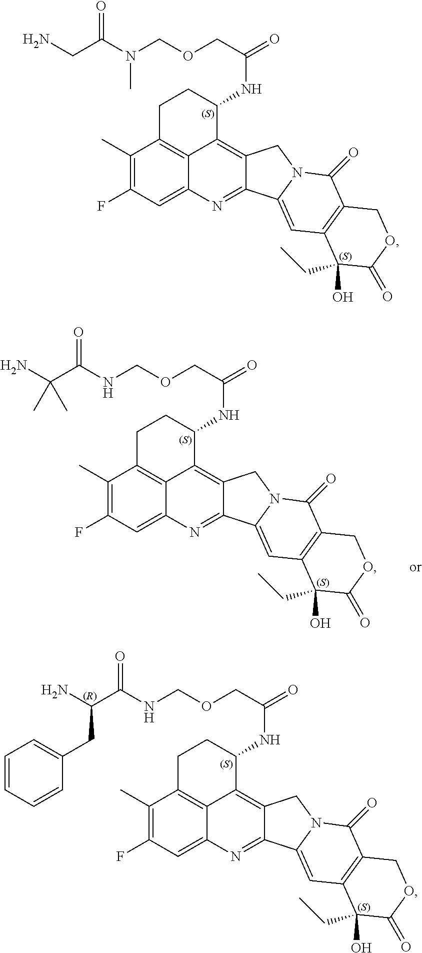

which is conjugated to the antibody or the antigen-binding fragment via the amino group.

In one embodiment, said antibody or said antigen-binding fragment thereof is conjugated to a compound having a structure according to Formula (II)

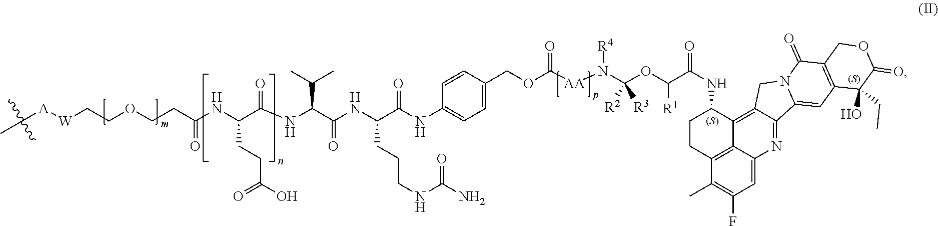

or a pharmaceutically acceptable salt thereof, wherein R¹, R², R³, and R⁴ are independently hydrogen or a C₁₋₅ alkyl;
A is a click chemistry adduct;
W is NH, O, CO, CH₂, a phenyl, or a combination of two or more thereof;
AA is a natural or a nonnatural amino acid;
m is an integer from 0 to 8;
n is 0 or 1;
p is an integer from 1 to 6, and

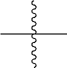

indicates the point of attachment to the antibody or the antigen-binding fragment thereof, directly or via a linker.

In one embodiment, the click chemistry adduct is a product of a copper-free click chemistry reaction selected from:
(a) strain-promoted azide/dibenzocyclooctyne-amine (DBCO) click chemistry;
(b) inverse electron demand Diels-Alder (IED-DA) tetrazine/trans-cyclooctene (TCO) click chemistry;
(c) inverse electron demand Diels-Alder (IED-DA) tetrazine/norbonene click chemistry;
(d) Diels-Alder maleimide/furan click-chemistry;
(e) Staudinger ligation; and
(f) nitrile-oxide/norbonene cycloaddition click chemistry.

In one non-limiting embodiment, the click chemistry adduct is a product of a strain-promoted azide/dibenzocyclooctyne-amine (DBCO) click chemistry reaction. In another embodiment, the click chemistry adduct is a product of inverse electron demand Diels-Alder (IED-DA) tetrazine/trans-cyclooctene (TCO) click chemistry reaction.

In one embodiment, the click chemistry adduct comprises a triazole. In another embodiment, the click chemistry adduct comprises a diazine.

In one embodiment, the click chemistry adduct is selected from the group consisting of:

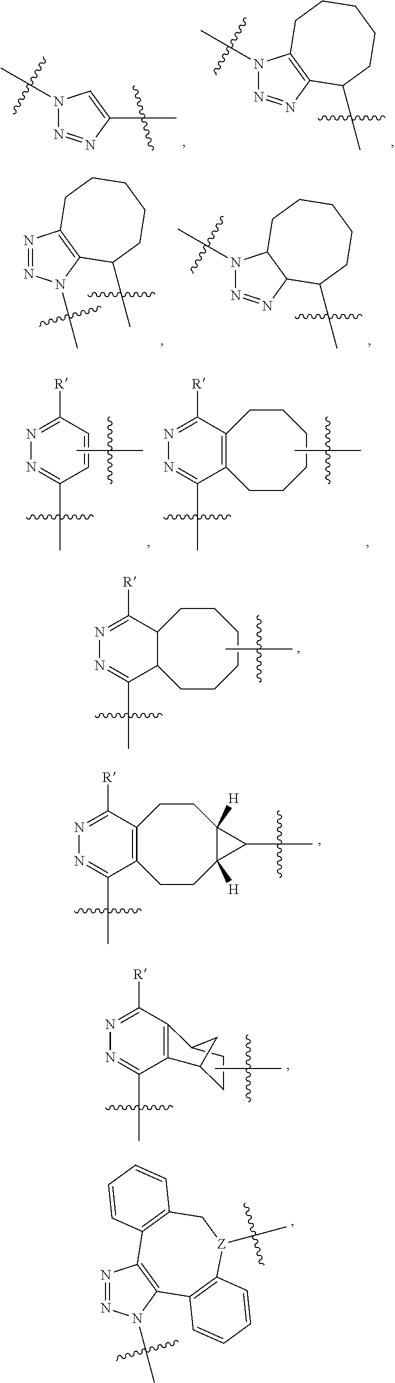

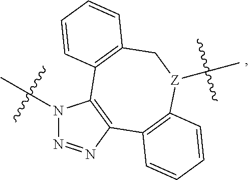

and any regio-isomers or entantiomers thereof, where R′ is H or a C₁₋₃ alkyl and Z is C or N.

In one embodiment, the click chemistry adduct is

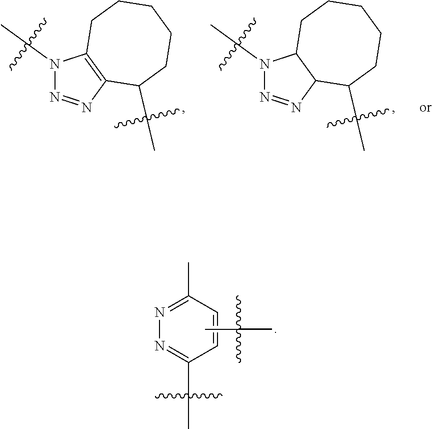

In one embodiment, R¹ is H.

In one embodiment, R² is H. In one embodiment, R³ is H. In one embodiment, R² and R³ are both Hs.

In one embodiment, R⁴ is H. In another embodiment, R⁴ is a C₁₋₅ alkyl. In one particular embodiment, R⁴ is a C₁ alkyl (a methyl).

In one embodiment, W is O. In one embodiment, W is NH. In one embodiment, W is CO. In one embodiment, W is CH₂. In one embodiment, W is a phenyl. In one embodiment, W is OCH₂. In one embodiment, W is —OCH₂—CO-NH—. In one embodiment, W is —O—CO—NH—. In one embodiment, W is

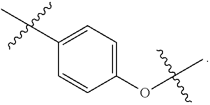 [SMILES: *Oc1ccc(*)cc1]

In one embodiment, m is 0. In another embodiment, m is 1. In another embodiment, m is 2. In another embodiment, m is 3. In another embodiment, m is 4. In another embodiment, m is 5. In another embodiment, m is 6. In another embodiment, m is 7. In another embodiment, m is 8.

In one particular embodiment, m is 4.

In one embodiment, n is 0. In another embodiment, n is 1.

In one embodiment, p is 1. In another embodiment, p is 2, i.e., [AA]₂ is a peptide dimer of two amino acids. In another embodiment, p is 3. In another embodiment, p is 4. In another embodiment, p is 5. In another embodiment, p is 6. In any embodiment where p is greater than one, amino acids may be the same or different from each other. In one embodiment, p is 2 and the two amino acids are different from each other.

In one embodiment, p is 1 and the amino acid is a natural amino acid. In one embodiment, p is 1 and the natural amino acid is selected from glycine, alanine, valine, leucine, isoleucine, phenylalanine, tyrosine, threonine, lysine, asparagine, glutamine, aspartic acid, and glutamic acid. In one embodiment, p is 1 and the natural amino acid is selected from glycine, phenylalanine, threonine, lysine, glutamine, and glutamic acid.

In one embodiment, p is 1 and the amino acid is a nonnatural amino acid. In one embodiment, p is 1 and the nonnatural amino acid is selected from the group consisting of an R-amino acid, an N-methyl amino acid,

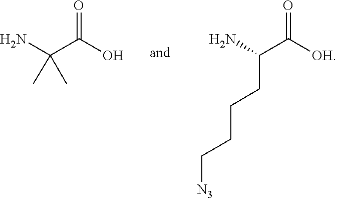

In another embodiment, p is 2, i.e., [AA]₂ is a peptide dimer of two amino acids. In one embodiment, p is 2 and both amino acids are glycines.

In one embodiment, said compound of Formula (II) comprises a compound having structure selected from the group consisting of:

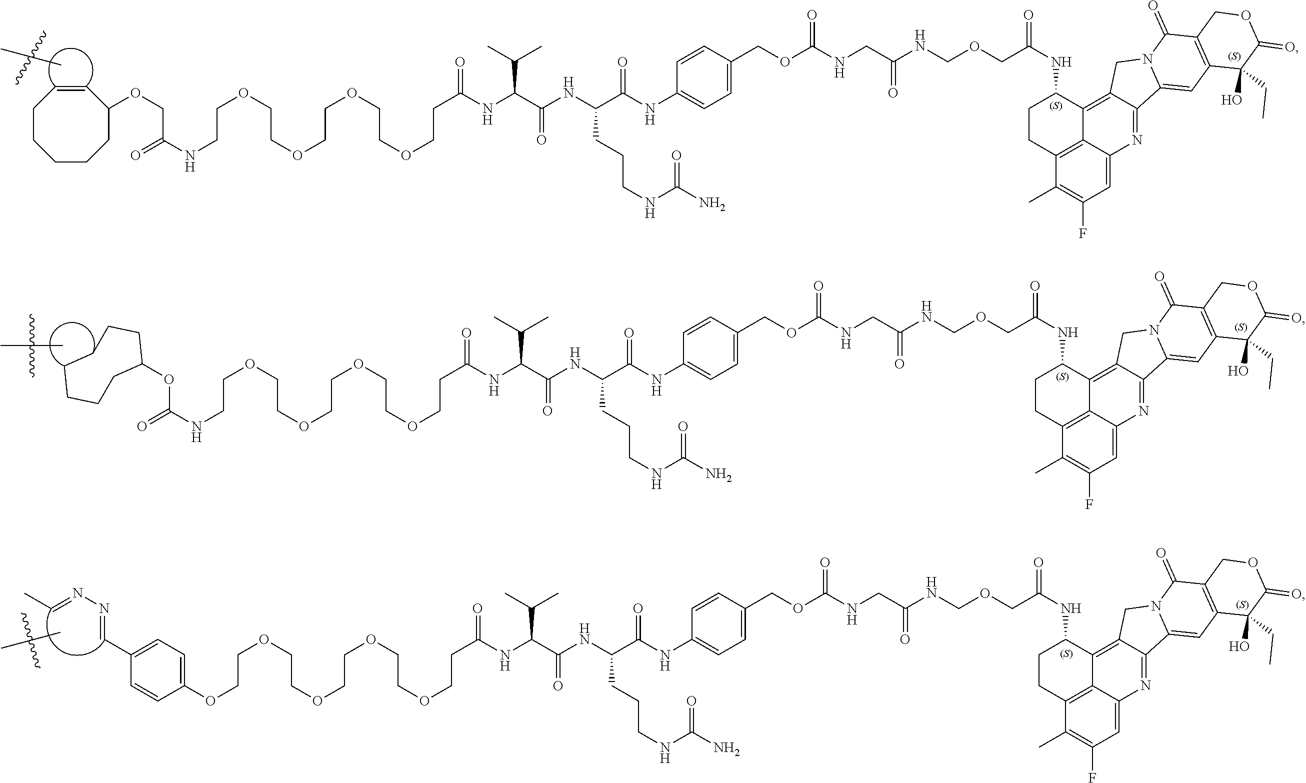

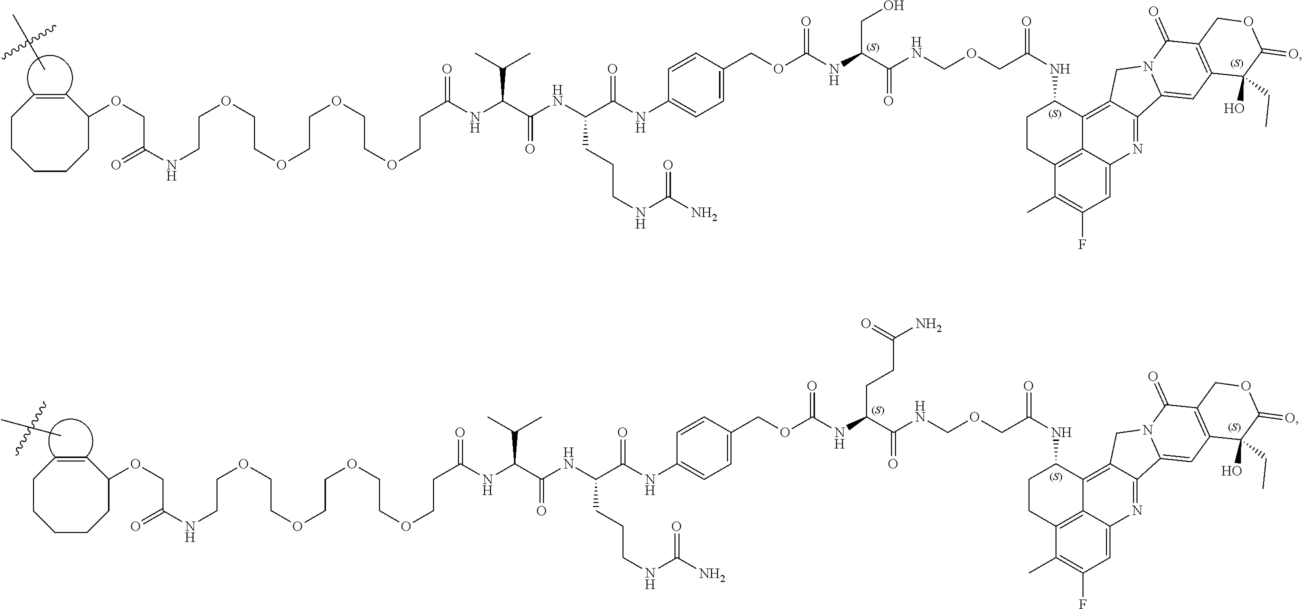

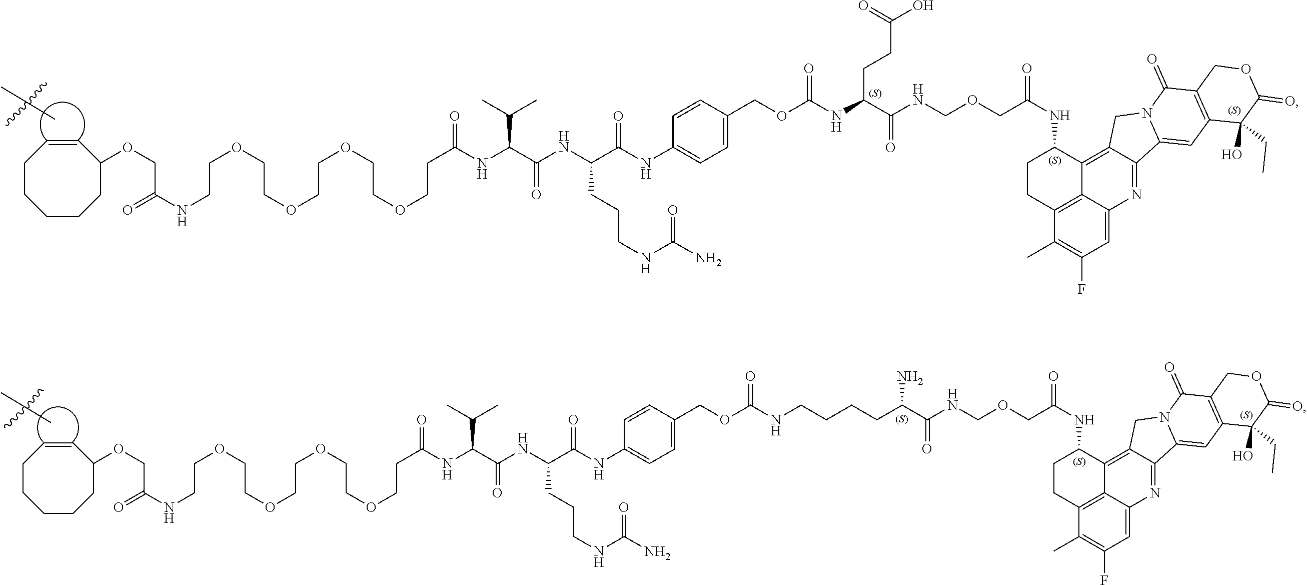

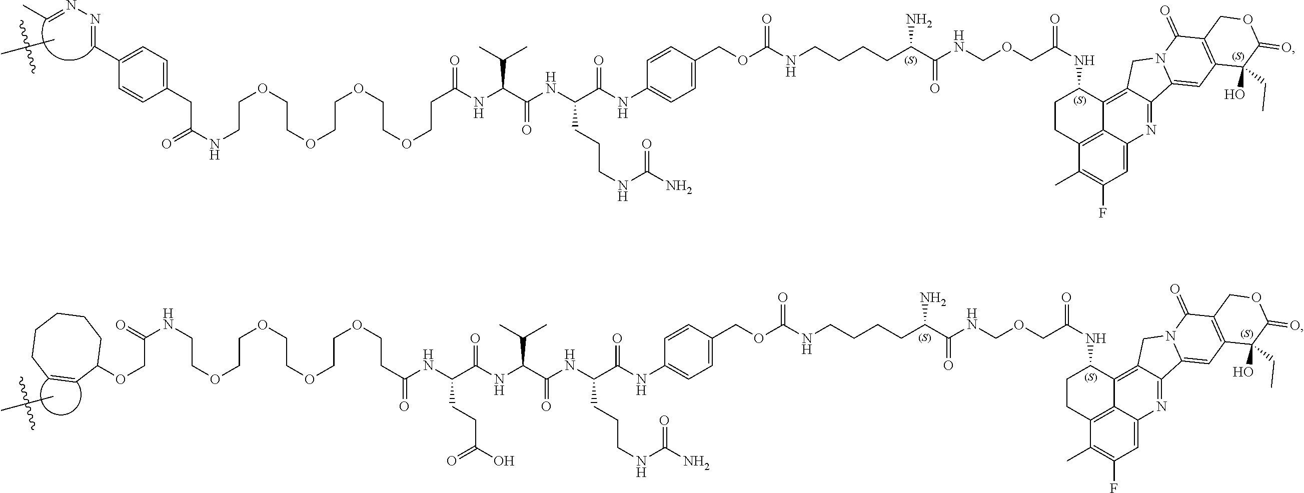

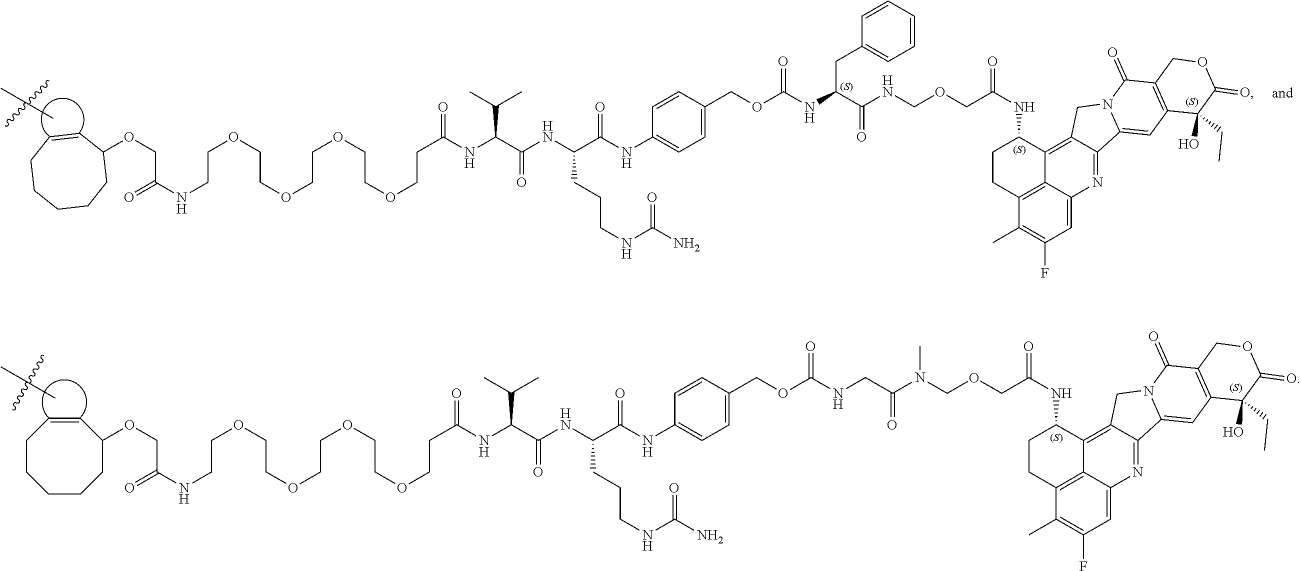

In one embodiment, said compound of Formula (II) comprises

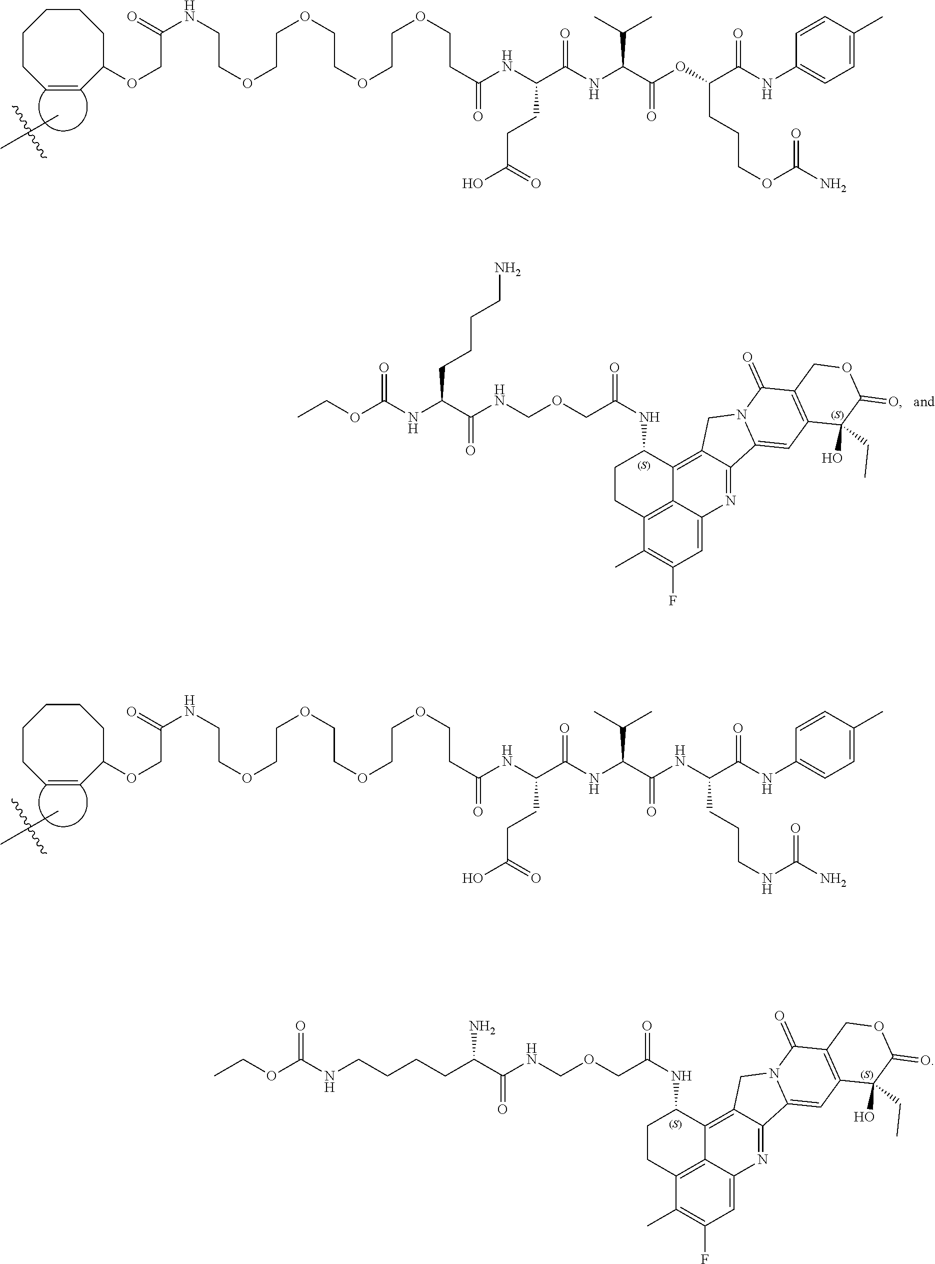

In one aspect, presented herein is an antibody-drug conjugate having a structure according to Formula (III)

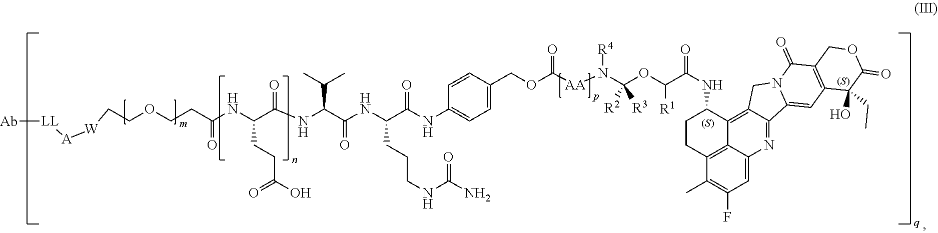

or a pharmaceutically acceptable salt thereof, wherein
Ab is an antibody or an antigen-binding fragment thereof;
R¹, R², R³, and R⁴ are independently hydrogen or a C₁₋₅ alkyl;
A is a click chemistry adduct;
W is NH, O, CO, CH₂, a phenyl, or a combination of two or more thereof;
LL is a linker or a bond connecting said Ab and said A;
AA is a natural or a nonnatural amino acid;
m is an integer from 0 to 8;
n is 0 or 1;
p is an integer from 1 to 6;
and q is an integer from 1 to 10.

In one embodiment, the click chemistry adduct is a product of a copper-free click chemistry reaction selected from:
(a) strain-promoted azide/dibenzocyclooctyne-amine (DBCO) click chemistry;
(b) inverse electron demand Diels-Alder (IED-DA) tetrazine/trans-cyclooctene (TCO) click chemistry;
(c) inverse electron demand Diels-Alder (IED-DA) tetrazine/norbonene click chemistry;
(d) Diels-Alder maleimide/furan click-chemistry;
(e) Staudinger ligation; and
(f) nitrile-oxide/norbonene cycloaddition click chemistry.

In one non-limiting embodiment, the click chemistry adduct is a product of a strain-promoted azide/dibenzocyclooctyne-amine (DBCO) click chemistry reaction. In another embodiment, the click chemistry adduct is a product of inverse electron demand Diels-Alder (IED-DA) tetrazine/trans-cyclooctene (TCO) click chemistry reaction.

In one embodiment, the click chemistry adduct comprises a triazole. In another embodiment, the click chemistry adduct comprises a diazine.

In one embodiment, the click chemistry adduct is selected from the group consisting of:

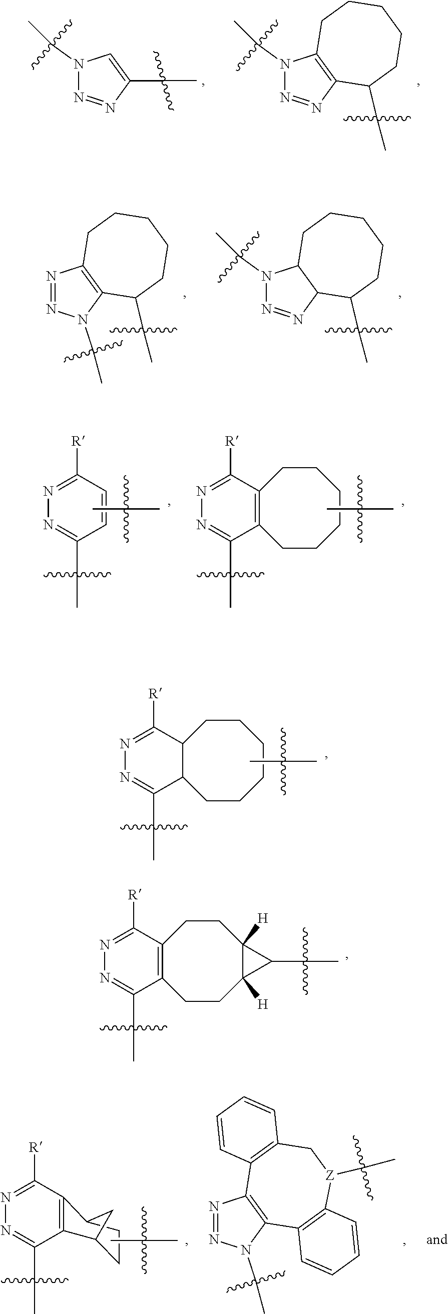

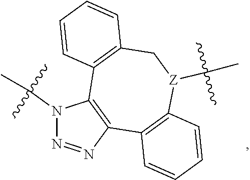

where R′ is H or a C₁₋₃ alkyl and Z is C or N.

In one embodiment, the click chemistry adduct is

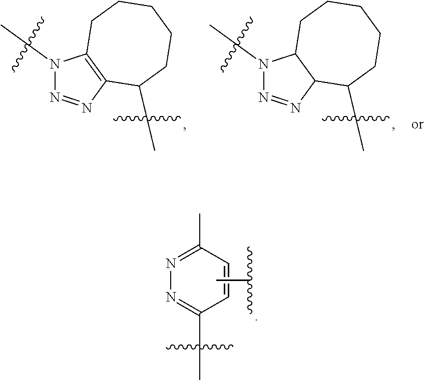

In one embodiment, R¹ is H.

In one embodiment, R² is H. In one embodiment, R³ is H. In one embodiment, R² and R³ are both Hs.

In one embodiment, R⁴ is H. In another embodiment, R⁴ is a C₁₋₅ alkyl. In one particular embodiment, R⁴ is a C₁ alkyl (a methyl).

In one embodiment, m is 0. In another embodiment, m is 1. In another embodiment, m is 2. In another embodiment, m is 3. In another embodiment, m is 4. In another embodiment, m is 5. In another embodiment, m is 6. In another embodiment, m is 7. In another embodiment, m is 8.

In one particular embodiment, m is 4.

In one embodiment, n is 0. In another embodiment, n is 1.

In one embodiment, p is 1. In another embodiment, p is 2, i.e., [AA]₂ is a peptide dimer of two amino acids. In another embodiment, p is 3. In another embodiment, p is 4. In another embodiment, p is 5. In another embodiment, p is 6. In any embodiment where p is greater than one, amino acids may be the same or different from each other. In one embodiment, p is 2 and the two amino acids are different from each other.

In one embodiment, p is 1 and the amino acid is a natural amino acid. In one embodiment, p is 1 and the natural amino acid is selected from glycine, alanine, valine, leucine, isoleucine, phenylalanine, tyrosine, threonine, lysine, asparagine, glutamine, aspartic acid, and glutamic acid. In one embodiment, p is 1 and the natural amino acid is selected from glycine, phenylalanine, threonine, lysine, glutamine, and glutamic acid.

In one embodiment, p is 1 and the amino acid is a nonnatural amino acid. In one embodiment, p is 1 and the nonnatural amino acid is selected from the group consisting of an R-amino acid, an N-methyl amino acid,

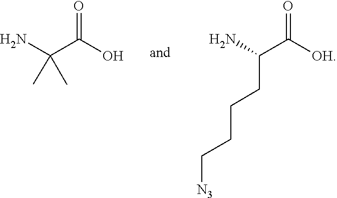

In another embodiment, p is 2, i.e., [AA]₂ is a peptide dimer of two amino acids. In one embodiment, p is 2 and both amino acids are glycines.

In one embodiment, LL is a bivalent or a multivalent linker selected from the group consisting of

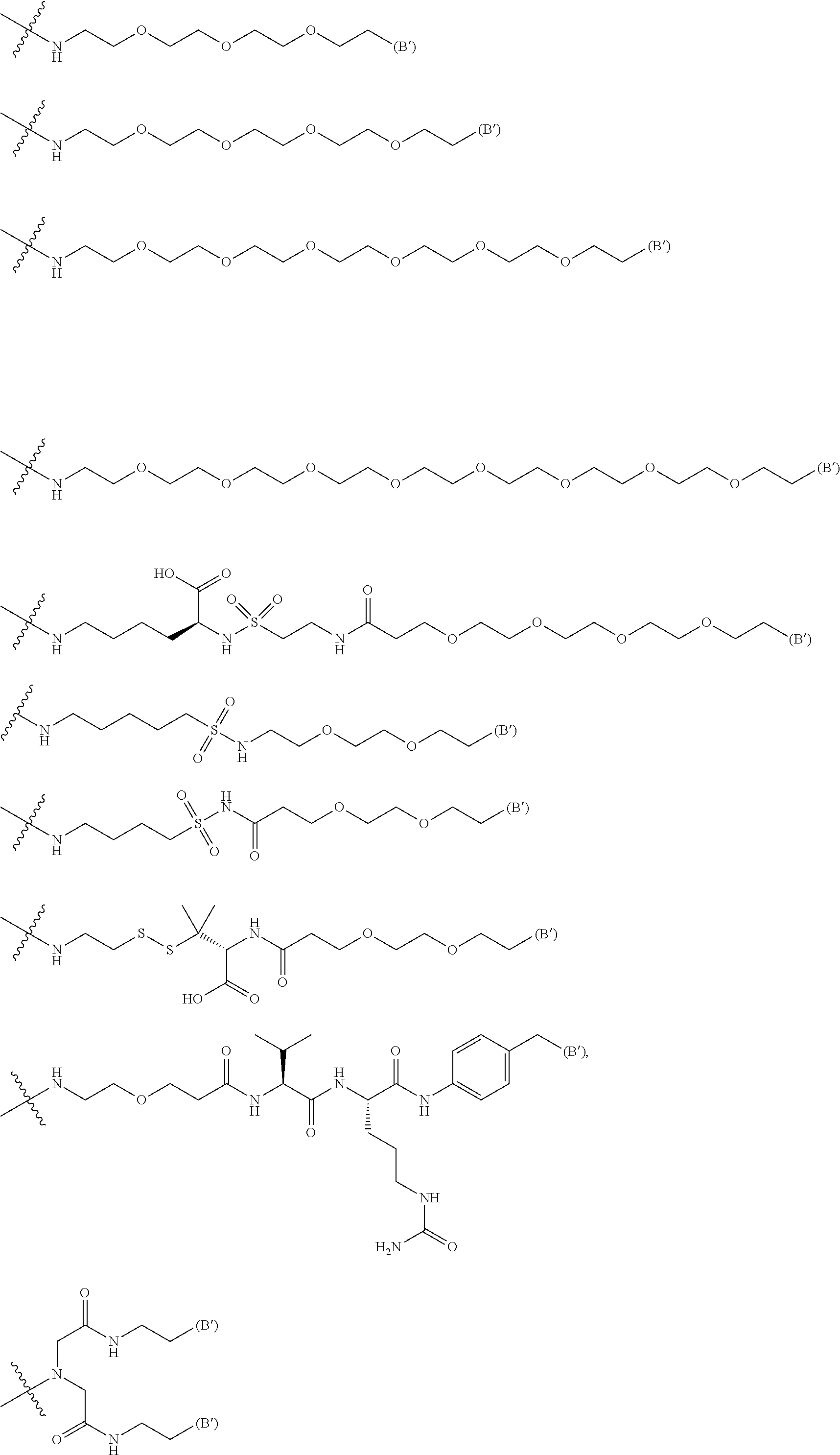

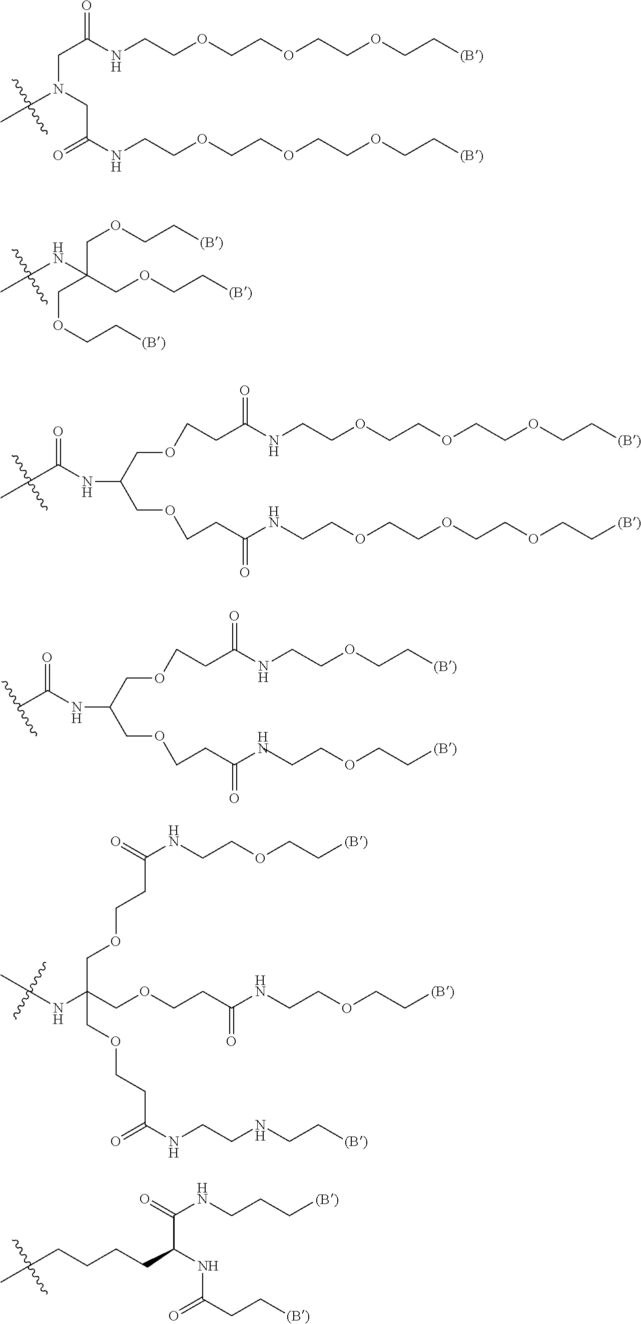

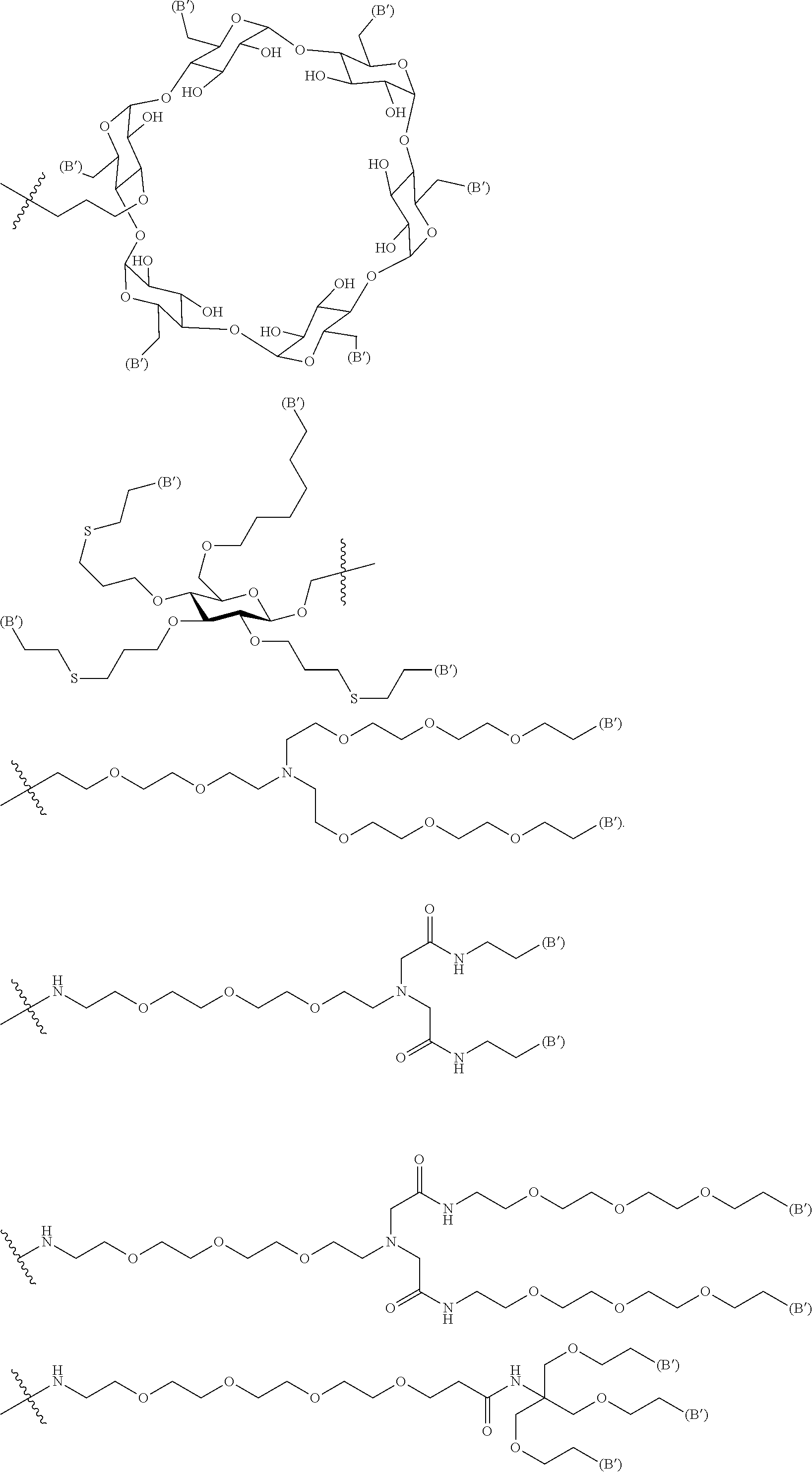

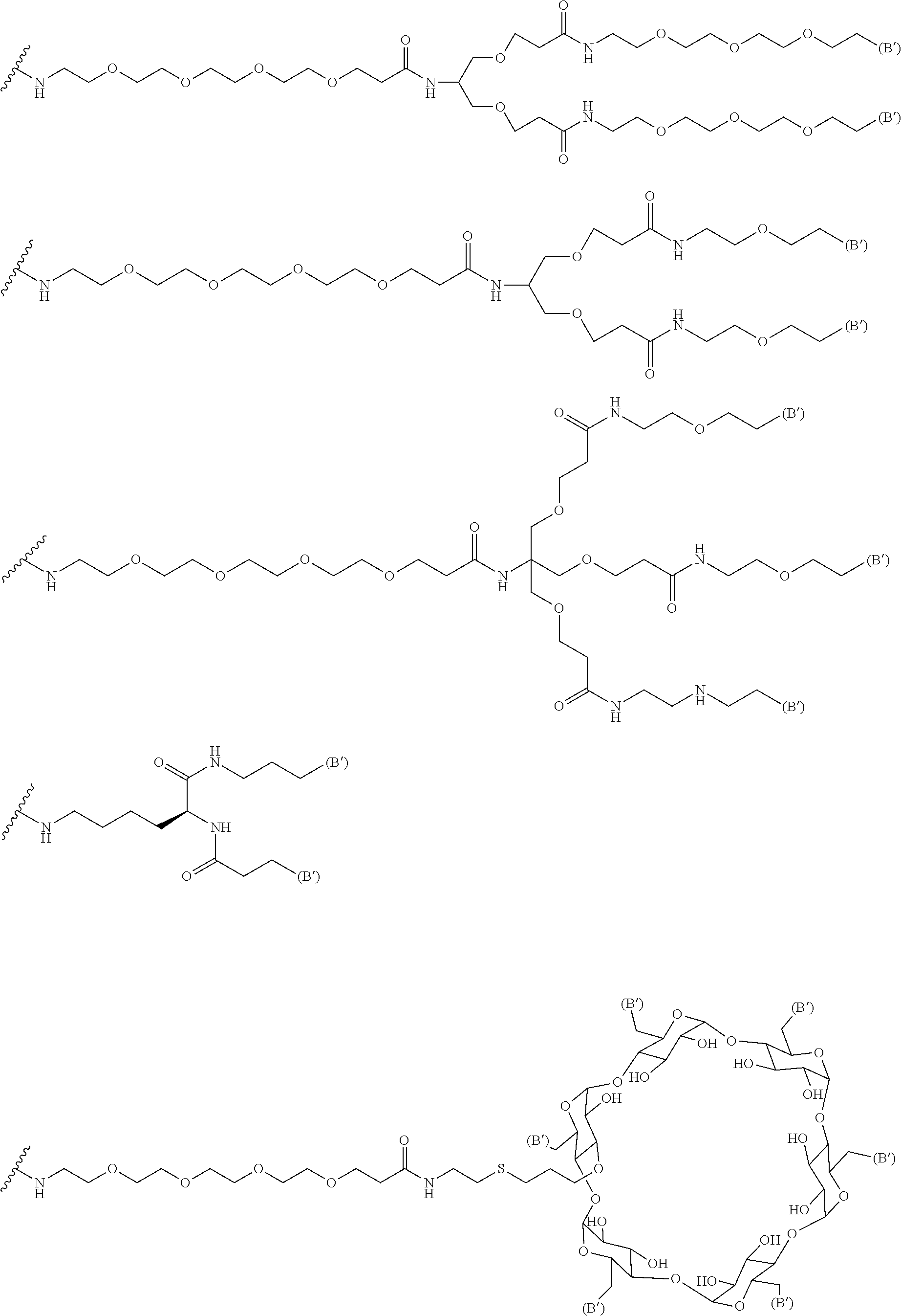

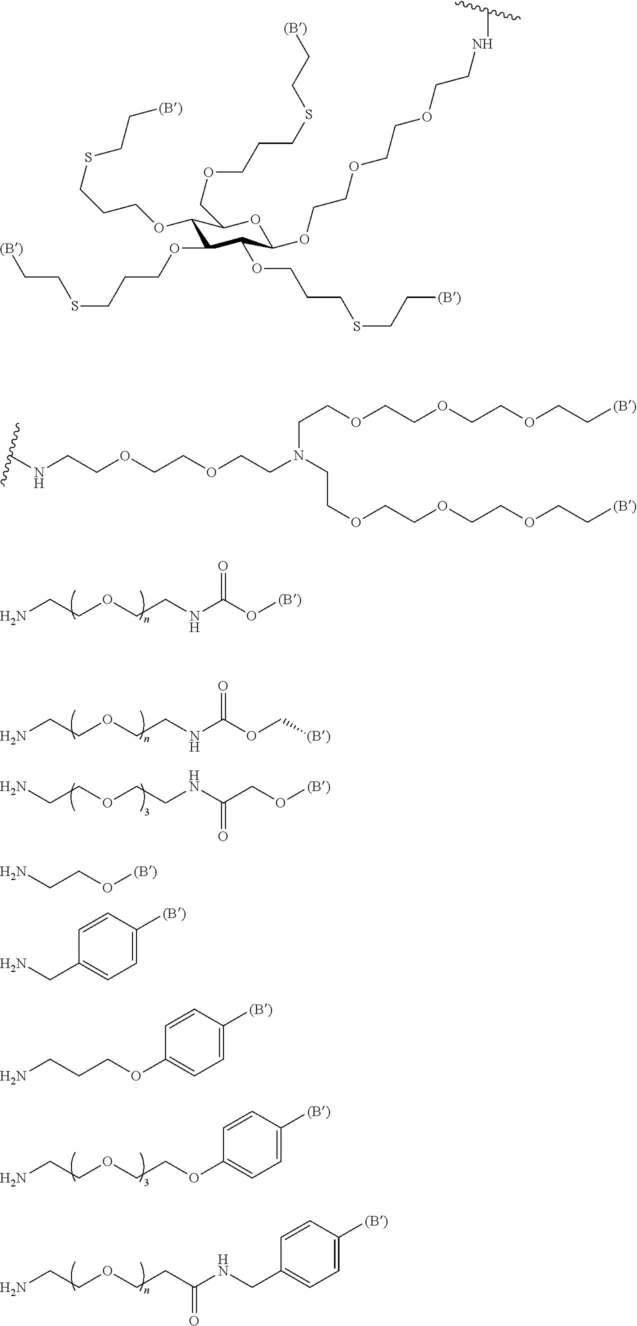

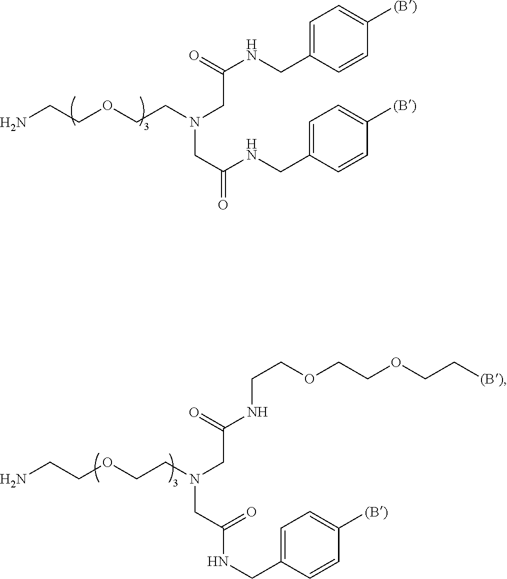

wherein (B′) is a point of attachment click chemistry adduct A and n is 0, 1, 2, 3, 4, 5, 6, 7, 8, 9, 10, 11, 12, 13, 14, or 15.

In one embodiment, LL is a bivalent or a multivalent linker selected from the group consisting of

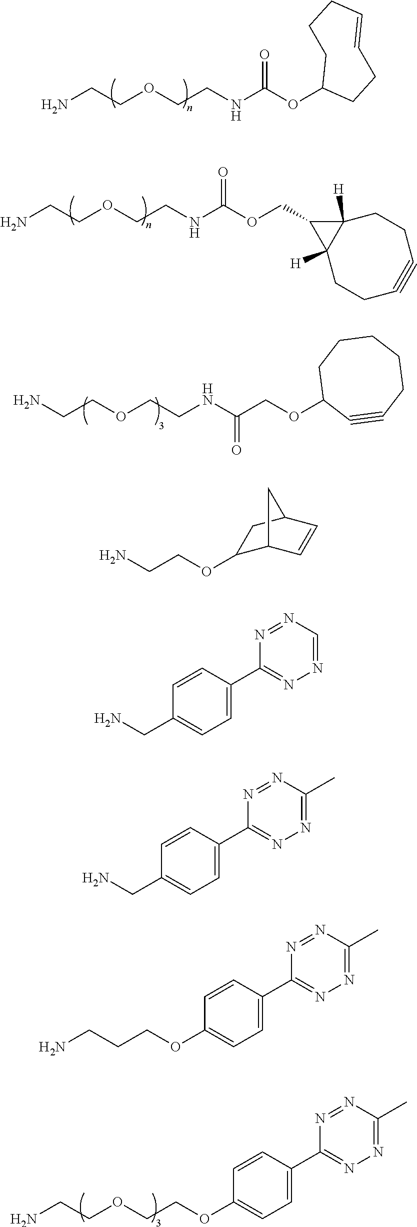

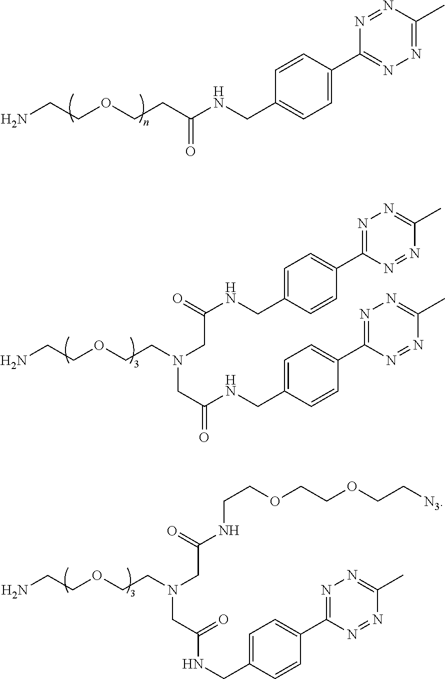

where n is 0,1, 2,3, 4, 5,6, 7, 8,9,10, 11, 12,13, 14, or 15.

In one embodiment, LL is a bivalent or a multivalent linker selected from the group consisting of

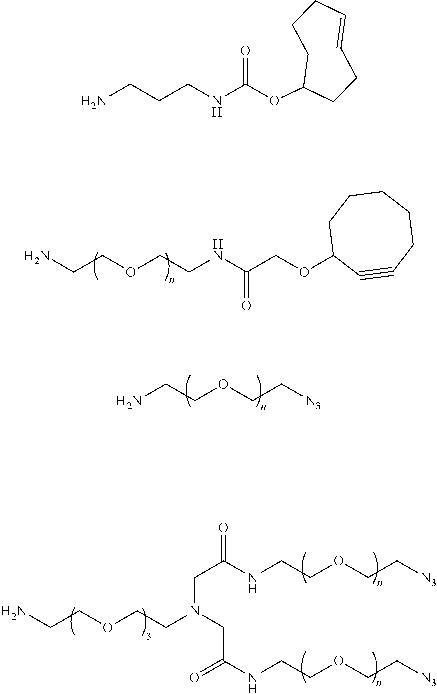

where n is 0, 1, 2, 3, 4, 5, 6, 7, 8, 9, 10, 11, 12, 13, 14, or 15.

In some embodiments, the linker comprises a self-immolative group. A self-immolative group, a self-immolative linker, or a self-immolative spacer, can be any such group known to those of skill in the art. Self-immolative linker displays an important role in the cascade mechanism of release of the compound linked. It is defined as a covalent group, which has the role of cleavaging two bonds between a protector group and a drug, in the case of drug delivery systems, after a stimuli. The stimuli may include enzyme triggers, chemical triggers, as pH, redox system, 1,4-, 1,6-, 1,8-eliminations, photodegradable triggers, multiple triggers, among others. The cascade of reactions of the self-immolative structural construct allows to control the release of a drug. In exemplary embodiments, the self-immolative group is p-aminobenzyl (PAB) or a derivative thereof. Useful derivatives include p-aminobenzyloxycarbonyl (PABC). Those of skill in the art will recognize that a self-immolative group is capable of carrying out a chemical reaction which releases the remaining atoms of a linker from a payload.

In one embodiment, q is 1. In another embodiment, q is 2. In another embodiment, q is 3. In another embodiment, q is 4. In another embodiment, q is 5. In another embodiment, q is 6. In another embodiment, q is 7. In another embodiment, q is 8. In another embodiment, q is 9. In another embodiment, q is 10.

In one embodiment, presented herein is an antibody-drug conjugate having a structure

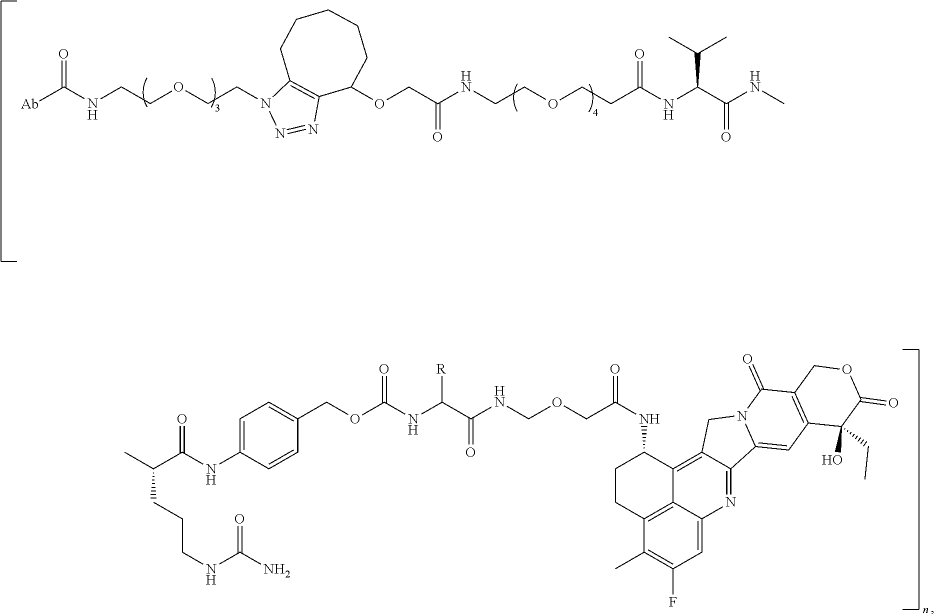

or a pharmaceutically acceptable salt thereof, wherein
Ab is an antibody or an antigen-binding fragment thereof;
R is a side chain of any natural or nonnatural amino acid; and
n is an integer from 1 to 5.

In another embodiment, presented herein is an antibody-drug conjugate having a structure

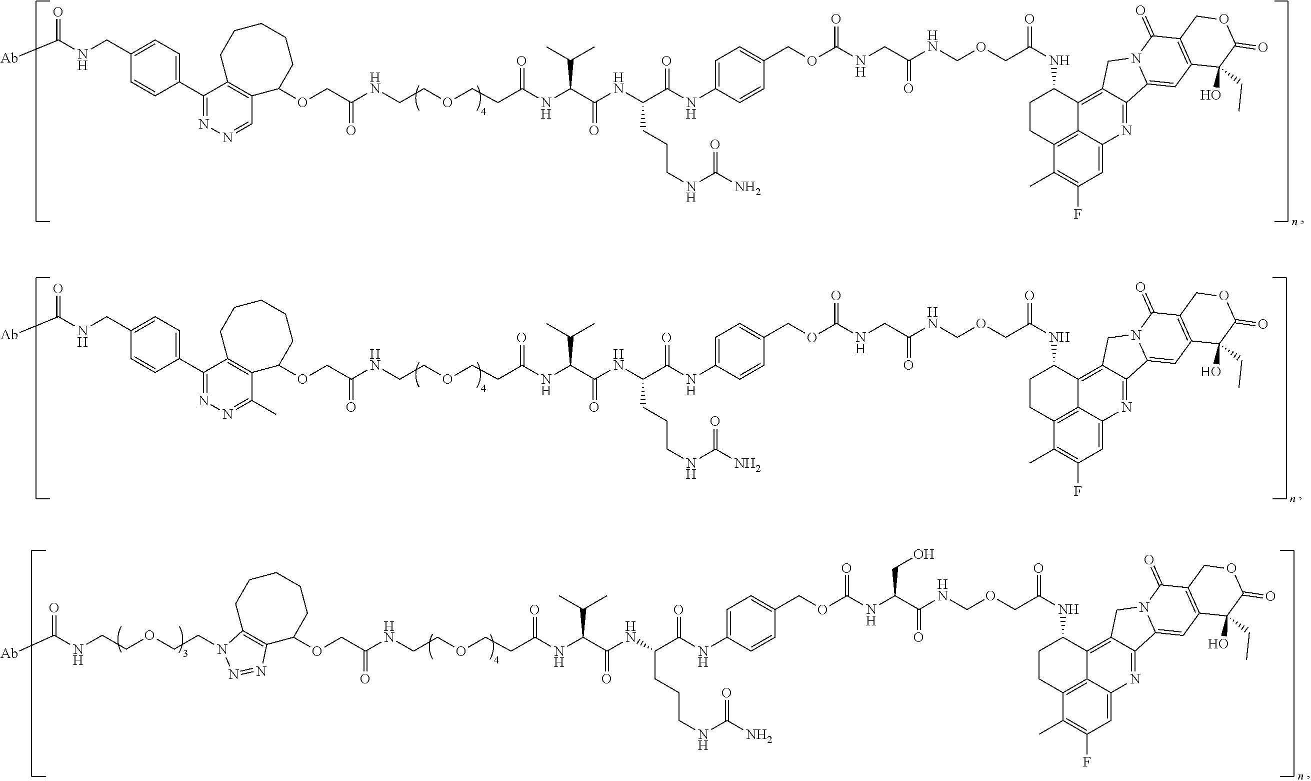

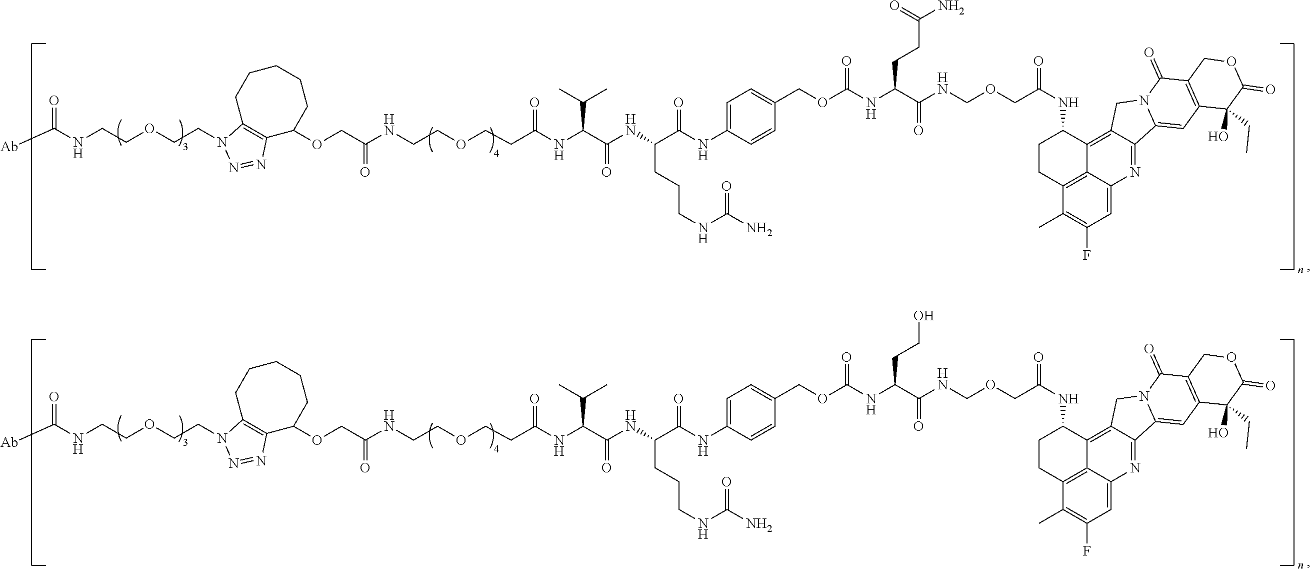

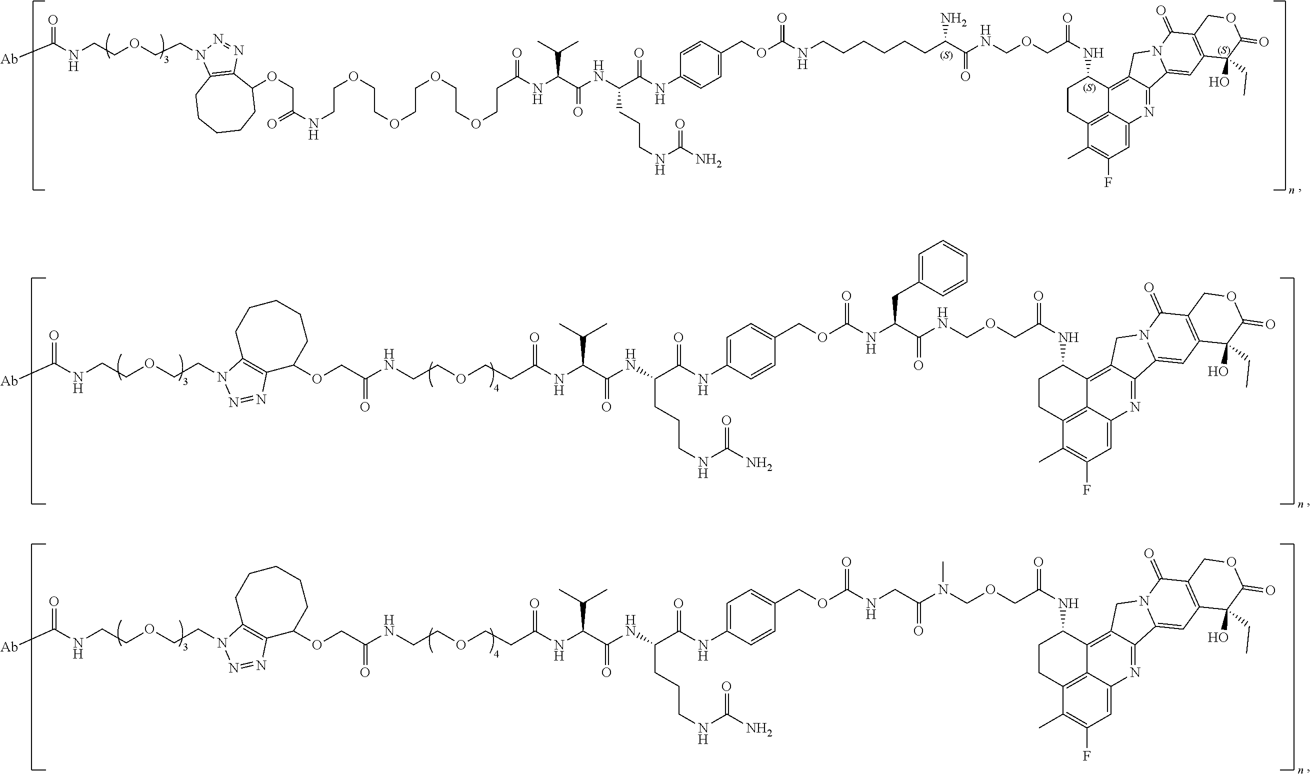

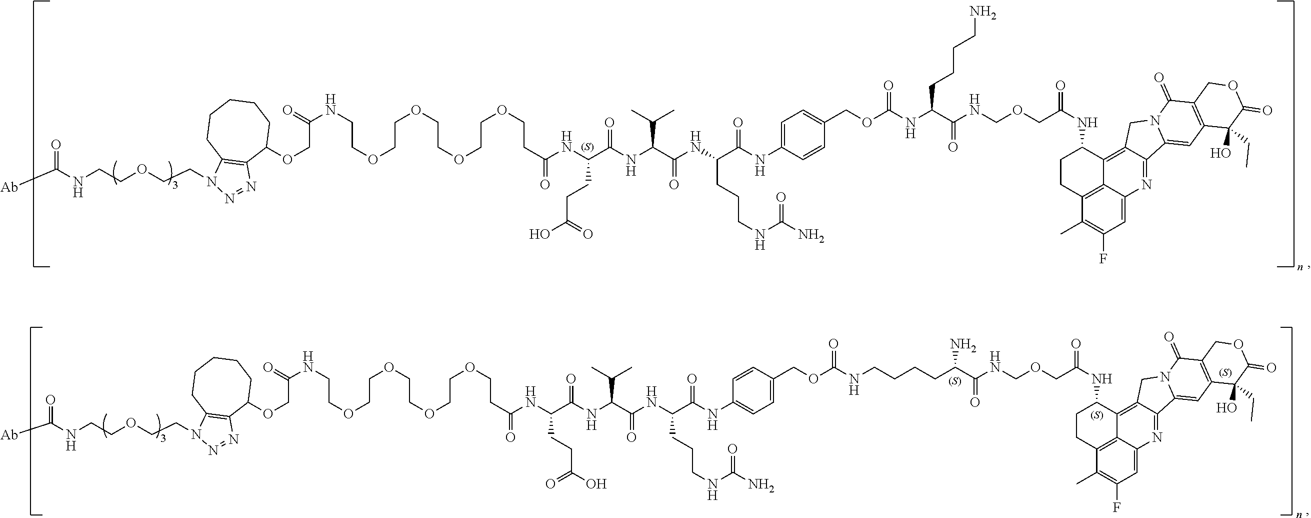

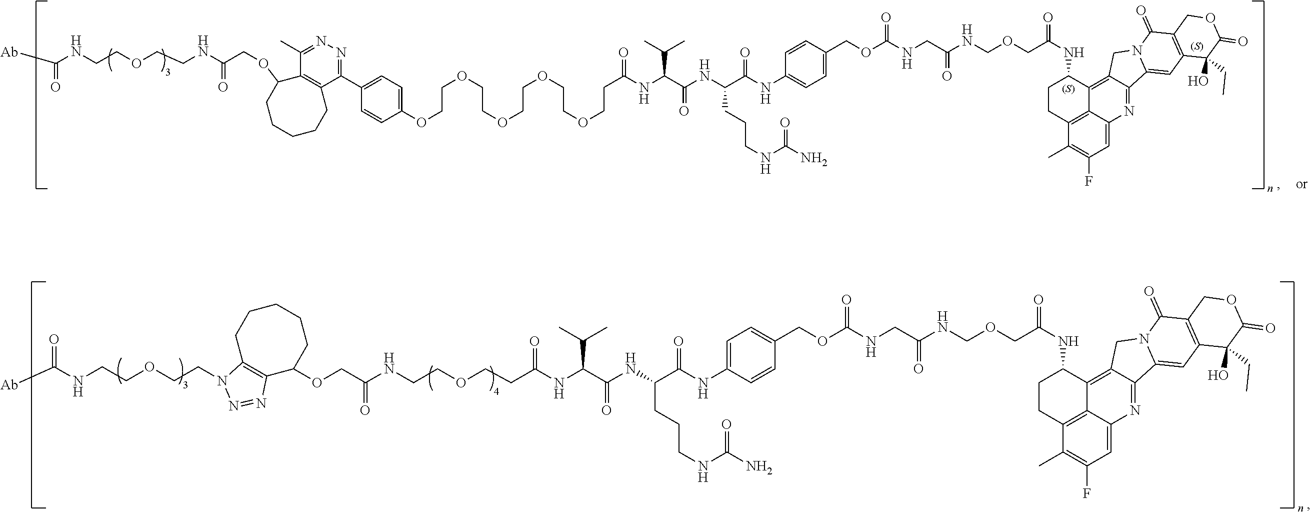

or a pharmaceutically acceptable salt thereof, wherein
Ab is an antibody or an antigen-binding fragment thereof; and
n is an integer from 1 to 5.

In one embodiment, presented herein is an antibody-drug conjugate having a structure

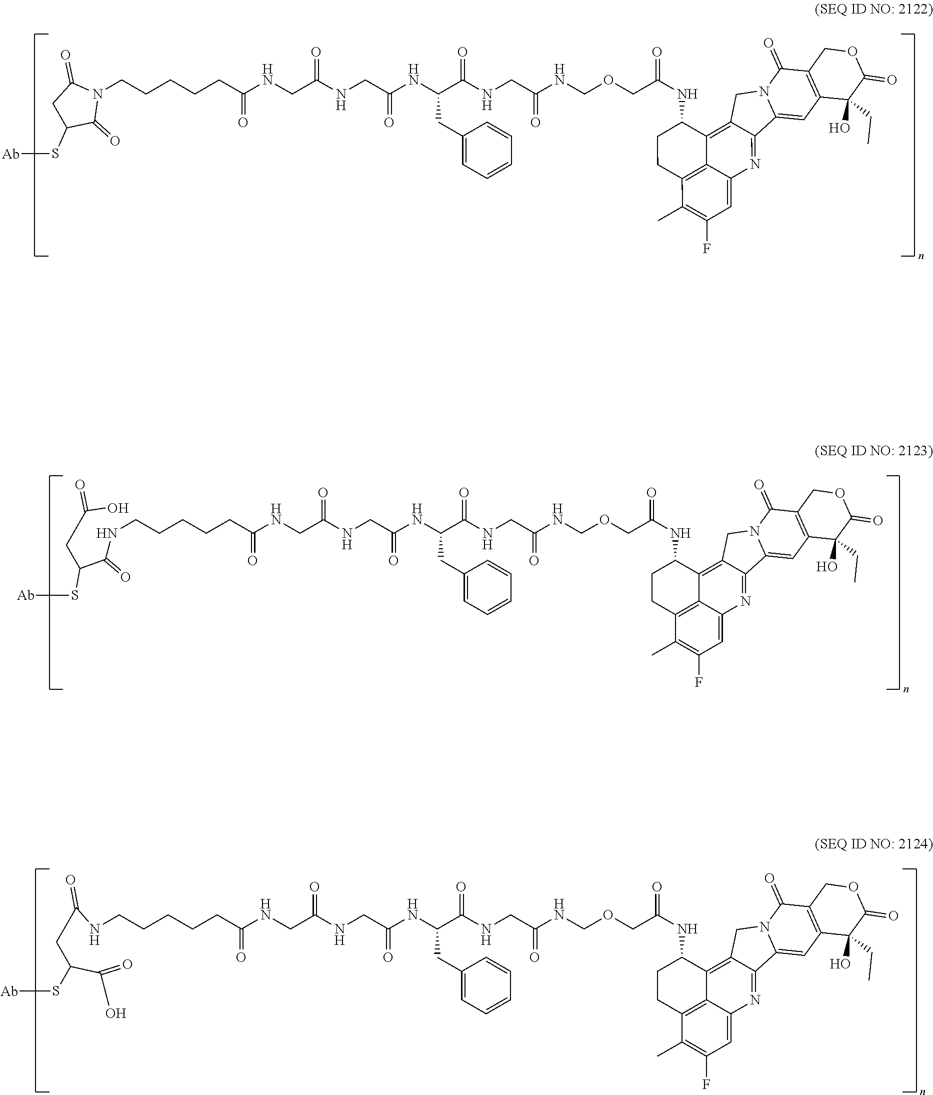

or a pharmaceutically acceptable salt thereof, wherein
Ab is an antibody or an antigen-binding fragment thereof; and
n is an integer from 1 to 5..

In one aspect, presented herein is an antibody-drug conjugate having a structure according to Formula (IVa or IVb):

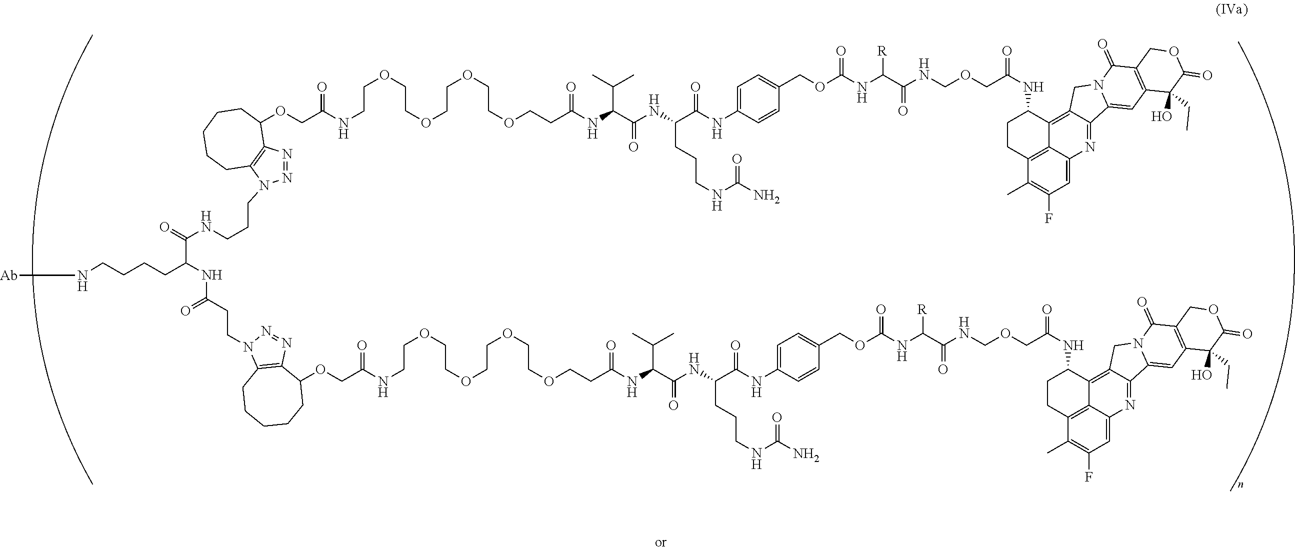

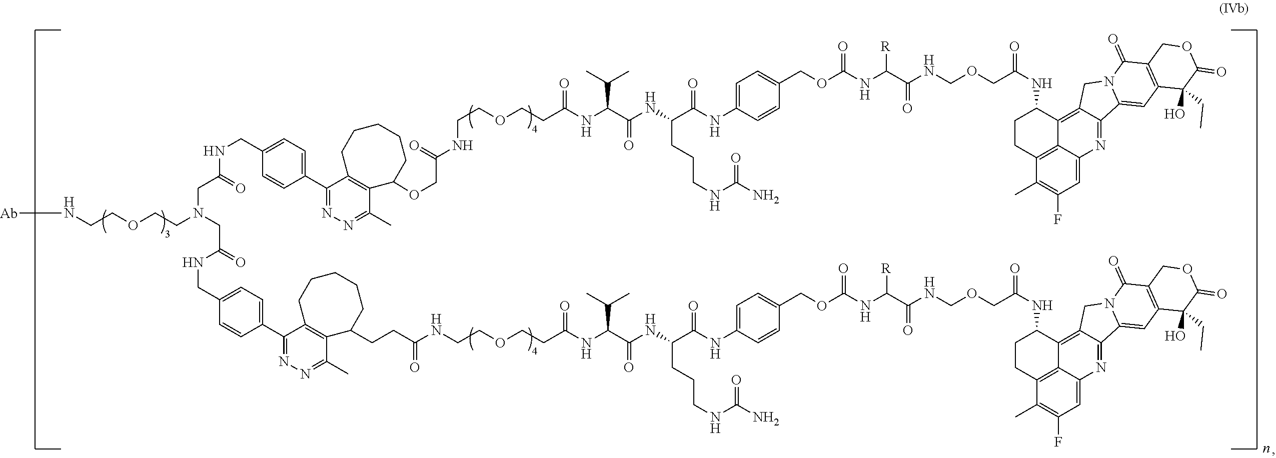

or a pharmaceutically acceptable salt thereof, wherein
Ab is an antibody or an antigen-binding fragment thereof;
R is a side chain of any natural or non-natural amino acid;
and n is an integer from 1 to 5.

In another aspect, the present disclosure provides an antibody-drug conjugate having a structure according to Formula (IVc, IVd, IVe, IVf, IVg, IVh, IVi, IVj, or IVk)

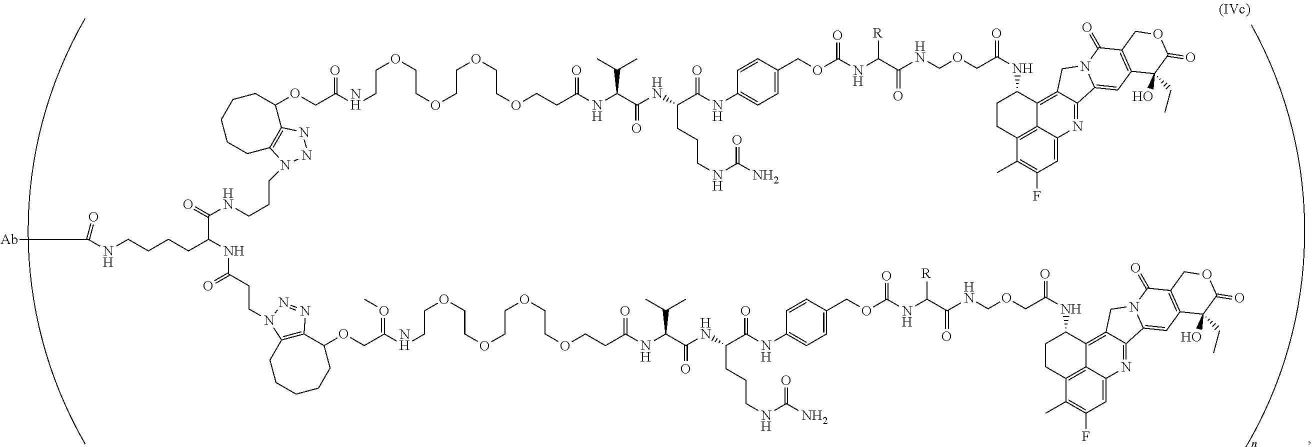

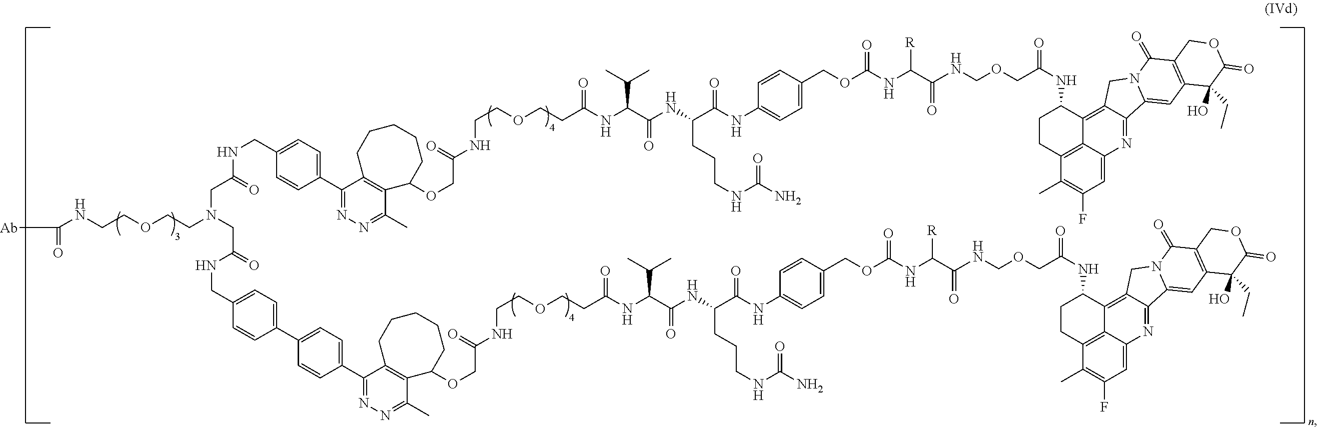

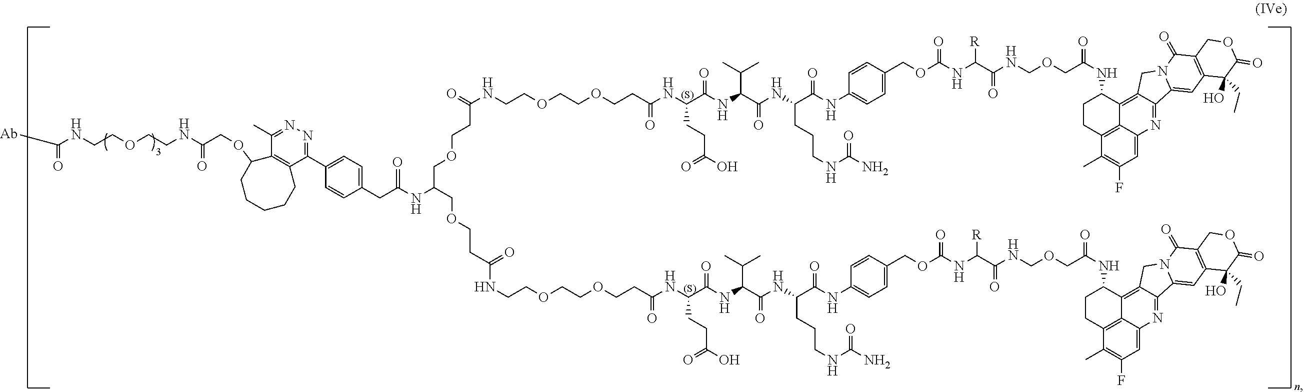

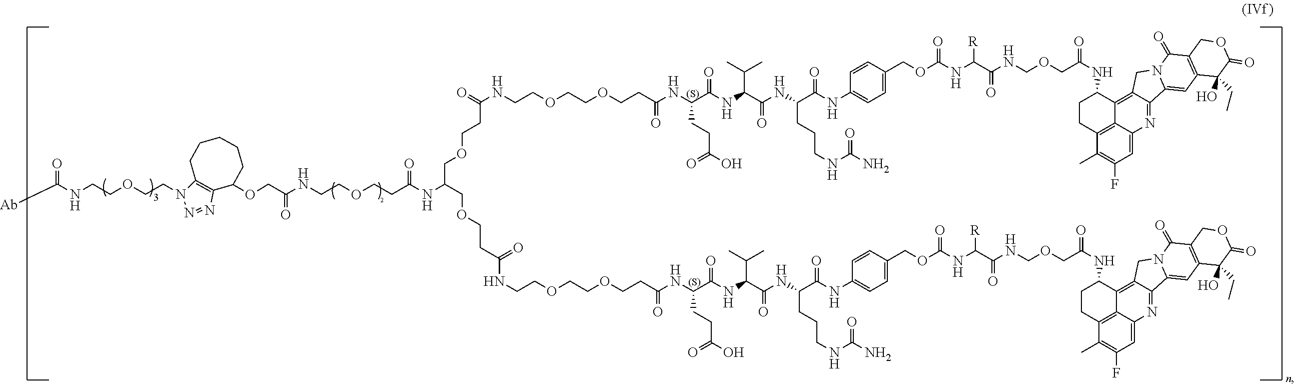

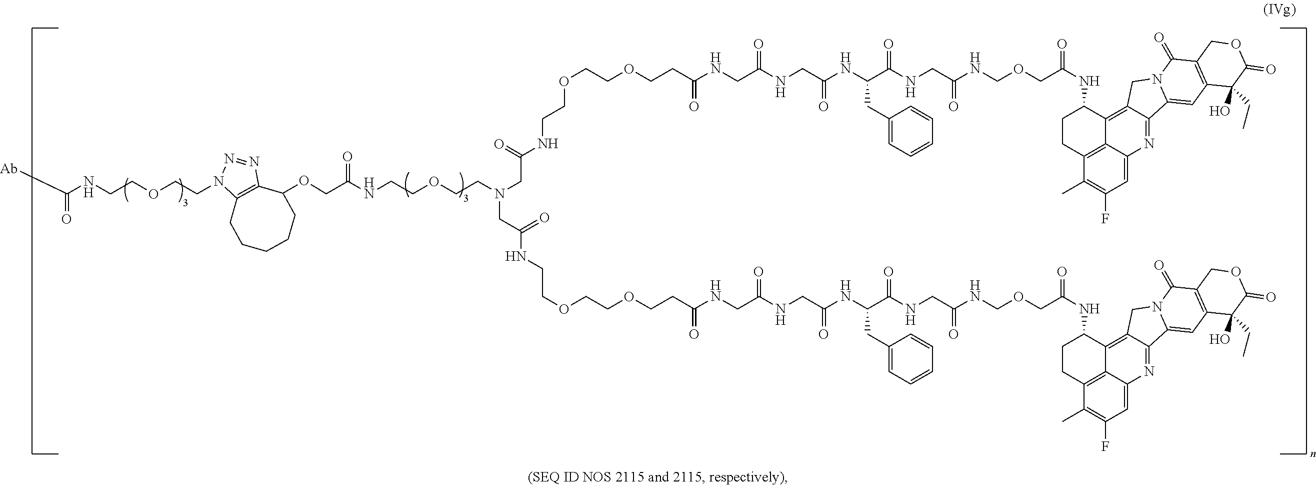

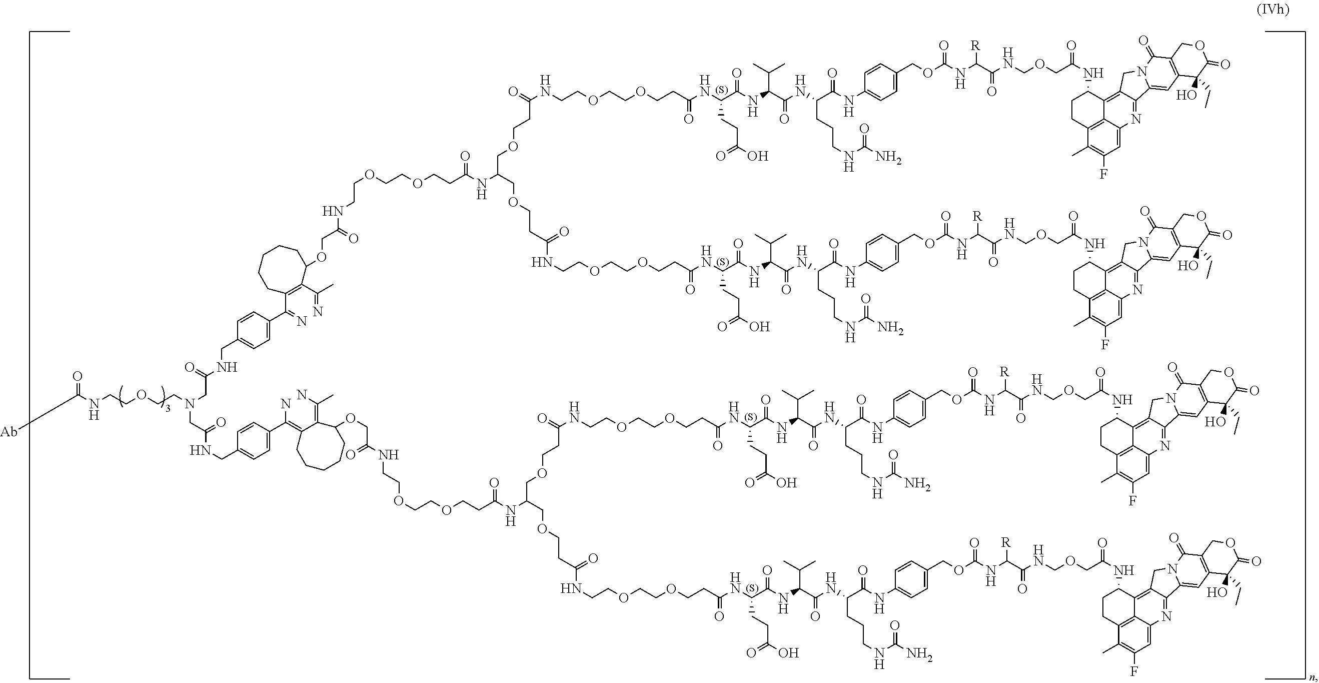

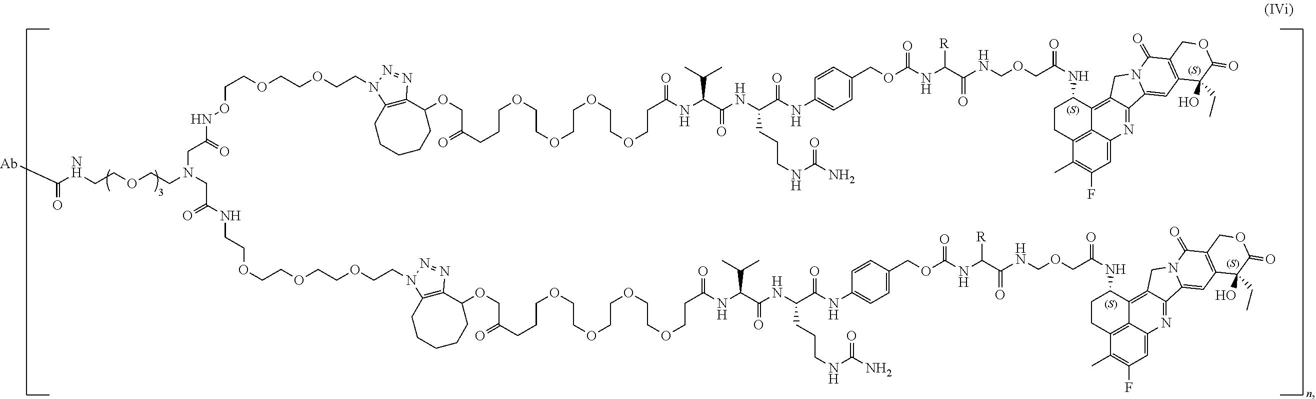

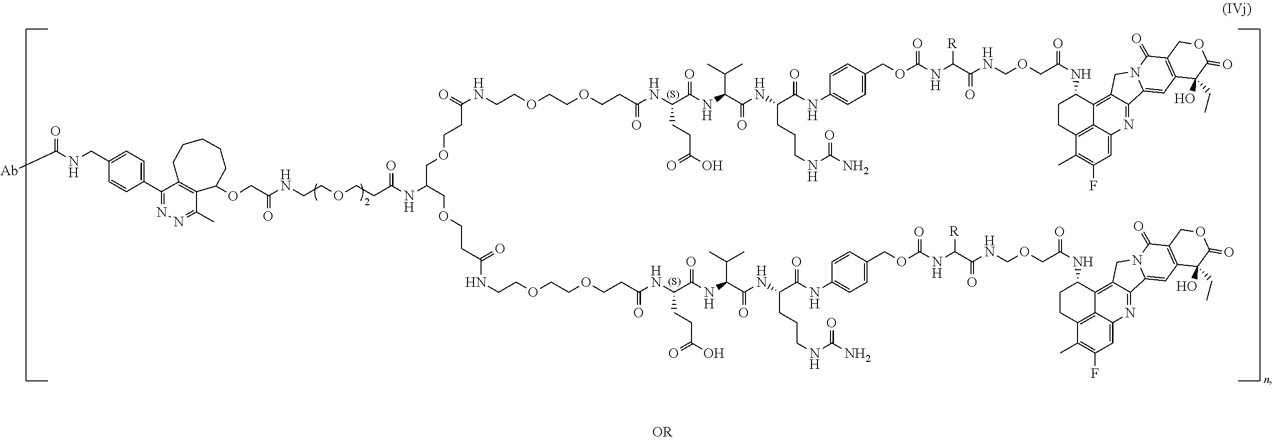

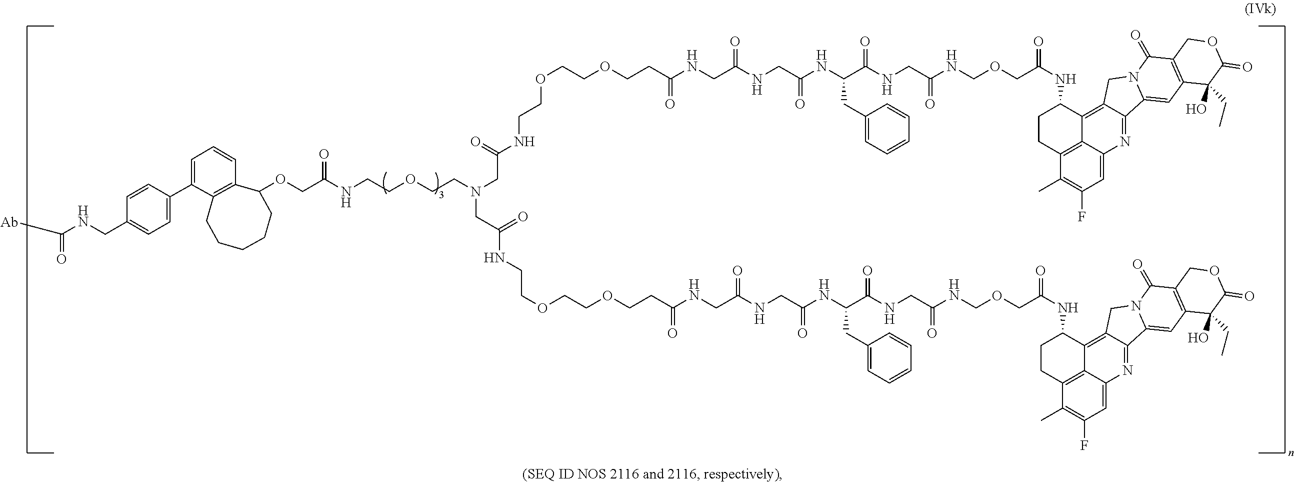

or a pharmaceutically acceptable salt thereof, wherein
Ab is an antibody or an antigen-binding fragment thereof;
R is a side chain of any natural or nonnatural amino acid;
and n is an integer from 1 to 5.

In one embodiment, R is a hydrogen.

In one embodiment, R is a side chain of a natural amino acid. In one embodiment, R is a side chain of a natural amino acid selected from glycine, alanine, valine, leucine, isoleucine, phenylalanine, tyrosine, threonine, lysine, asparagine, glutamine, aspartic acid, and glutamic acid. In one embodiment, R is a side chain of a natural amino acid selected from glycine, phenylalanine, threonine, lysine, glutamine, and glutamic acid.

In one embodiment, R is a side chain of a nonnatural amino acid. In one embodiment, R is a side chain of a nonnatural amino acid selected from the group consisting of an R-amino acid, an N-methyl amino acid,

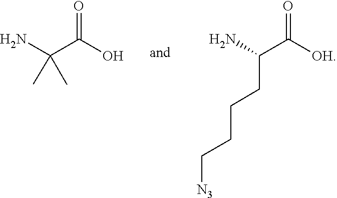

In one embodiment of any of the above, said antibody or said antigen-binding fragment thereof comprises Gln295 and/or Gln297 (i.e., a glutamine residue in position 295 and/or 297), and the payload (e.g., a prodrug of DXd) is conjugated to said antibody or antigen-binding fragment through the side chains of Gln295 and/or Gln297, directly or via a linker.

### Payloads

In certain embodiments, the payloads of the present disclosure are prodrugs of a topoisomerase I inhibitor. In certain embodiments, the payloads of the present disclosure are camptothecin analogs and/or derivatives.

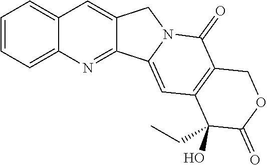 [SMILES: CC[C@@]1(O)C(=O)OCc2c1cc1n(c2=O)Cc2cc3ccccc3nc2-1]

### Camptothecin

Camptothecin (CPT), shown above, is a topoisomerase poison. It was discovered in 1966 by M. E. Wall and M. C. Wani in systematic screening of natural products for anticancer drugs. It was isolated from the bark and stem of Camptotheca acuminata (Camptotheca, Happy tree), a tree native to China used as a cancer treatment in Traditional Chinese Medicine. Camptothecin showed remarkable anticancer activity in preliminary clinical trials. However, it has low solubility, so synthetic and medicinal chemists have developed numerous syntheses of camptothecin and various derivatives to increase the benefits of the chemical, with good results. Four camptothecin analogs have been approved and are used in cancer chemotherapy today: topotecan, irinotecan, belotecan, and deruxtecan (Dxd).

Trastuzumab deruxtecan (T-Dxd) is an antibody-drug conjugate that includes a human epidermal growth factor receptor 2 (HER2)-directed antibody trastuzumab and a topoisomerase I inhibitor conjugate deruxtecan (Dxd, a derivative of exatecan). It was approved for use in the United States in December 2019. Exatecan, shown below, is a camptothecin analog.

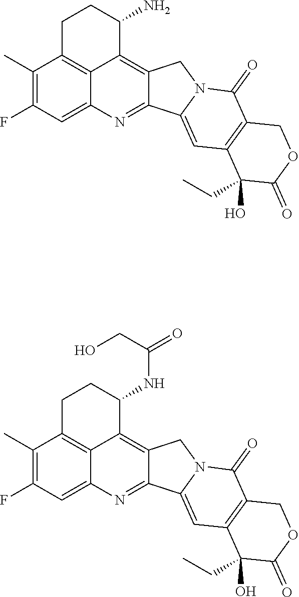 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)CO)CC3]

### Exatecan, Left, and Deruxtecan (Dxd), Right

In one embodiment, the payload of the present disclosure is a prodrug of deruxtecan (Dxd).

In certain embodiments, the payload of the present disclosure is a compound having the structure P-I:

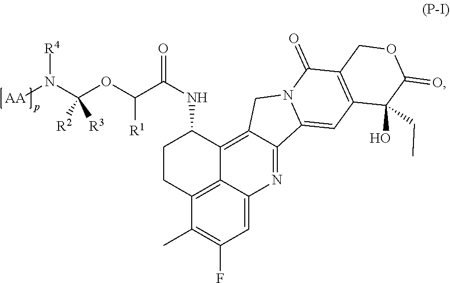

wherein R¹, R², R³, and R⁴ are independently hydrogen or a C₁₋₅ alkyl;
AA is a natural or a nonnatural amino acid; and
p is an integer from 1 to 6, or a pharmaceutically acceptable salt thereof.

In one embodiment, p is 1. In another embodiment, p is 2, i.e., [AA]₂ is a peptide dimer of two amino acids. In another embodiment, p is 3. In another embodiment, p is 4. In another embodiment, p is 5. In another embodiment, p is 6. In any embodiment where p is greater than one, amino acids may be the same or different from each other. In one embodiment, p is 2 and the two amino acids are different from each other.

In one embodiment, p is 1 and the amino acid is a natural amino acid. In one embodiment, p is 1 and the natural amino acid is selected from glycine, alanine, valine, leucine, isoleucine, phenylalanine, tyrosine, threonine, lysine, asparagine, glutamine, aspartic acid, and glutamic acid. In one embodiment, p is 1 and the natural amino acid is selected from glycine, phenylalanine, threonine, lysine, glutamine, and glutamic acid.

In one embodiment, p is 1 and the amino acid is a nonnatural amino acid. In one embodiment, p is 1 and the nonnatural amino acid is selected from the group consisting of an R-amino acid, an N-methyl amino acid,

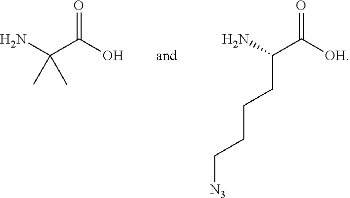

In another embodiment, p is 2, i.e., [AA]₂ is a peptide dimer of two amino acids. In one embodiment, p is 2 and both amino acids are glycines.

In one embodiment, R¹ is H.

In one embodiment, R² is H. In one embodiment, R³ is H. In one embodiment, R² and R³ are both Hs.

In one embodiment, R⁴ is H. In another embodiment, R⁴ is a C₁₋₅ alkyl. In one particular embodiment, R⁴ is a C₁ alkyl (a methyl).

In one embodiment, the compound of Formula (I) is selected from the group consisting of the compounds of Table 1.

TABLE 1
Structures of EXT, DXd, and the prodrugs of DXd according to embodiments of the
present disclosure

| | | | | | | | MS |
|---|---|---|---|---|---|---|---|
| | | | | | | HPLC | (M + |
| Cpd# | Structures | cLogP | MF | MW | Salt | purity | H) |
| Exa- tecan (EXT) | 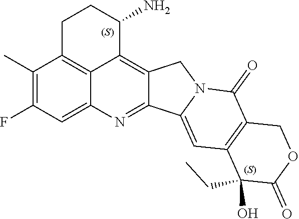 | 1.45 | C₂₄H₂₂FN₃O₄ | 435.46 | MsOH | 97 | 436.1 |
| DXd | 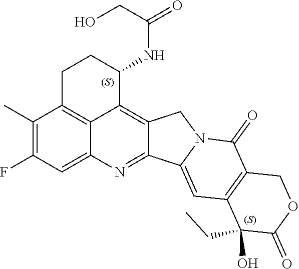 | 0.55 | C₂₆H₂₄FN₃O₆ | 493.49 | NA | 99 | 494.1 |
| P1 | 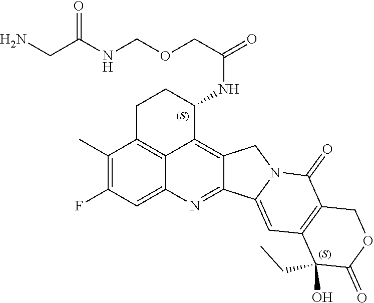 | −0.60 | C₂₉H₃₀FN₅O₇ | 579.59 | TFA | 99 | 580.2 |
| P2 | 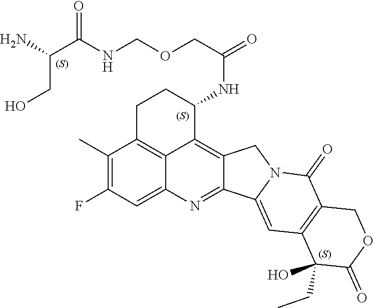 | −1.08 | C₃₀H₃₂FN₅O₈ | 609.61 | TFA | 95 | 610.2 |
| P3 | 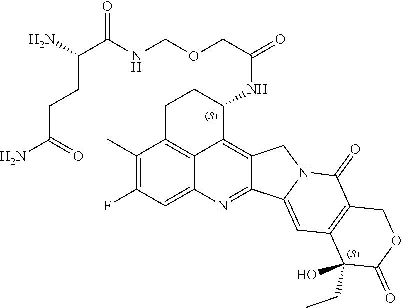 | −1.19 | C₃₂H₃₅FN₆O₈ | 650.66 | NA | 99 | 651.3 |
| P4 | 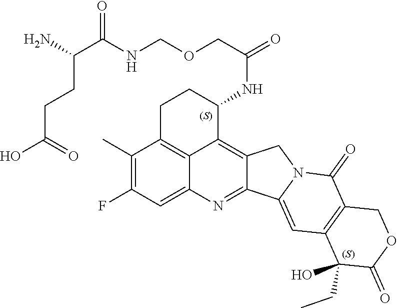 | −2.65 | C₃₂H₃₄FN₅O₉ | 651.65 | NA | 95 | 652.2 |
| P5 | 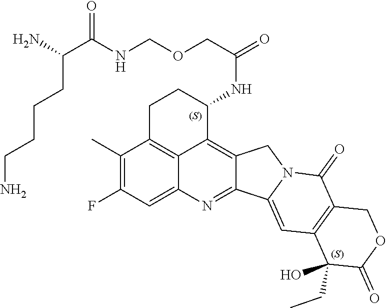 | −0.36 | C₃₃H₃₉FN₆O₇ | 650.71 | TFA | 99 | 651.3 |
| P6 | 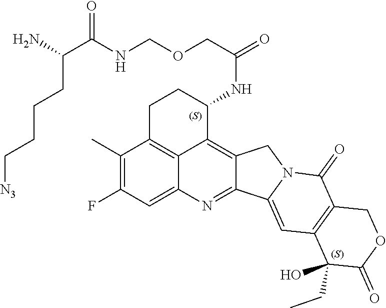 | 0.76 | C₃₃H₃₇FN₈O₇ | 676.71 | NA | 99 | 677.3 |
| P7 | 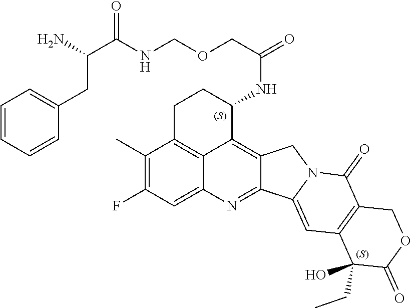 | 1.62 | C₃₆H₃₆FN₅O₇ | 669.71 | NA | 99 | 670.2 |
| P8 | 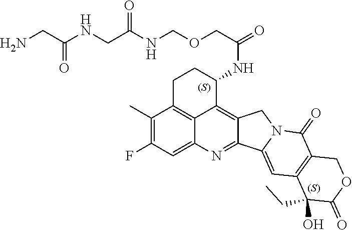 | −1.71 | C₃₁H₃₃FN₆O₈ | 636.64 | TFA | 99 | 637.3 |
| P9 | 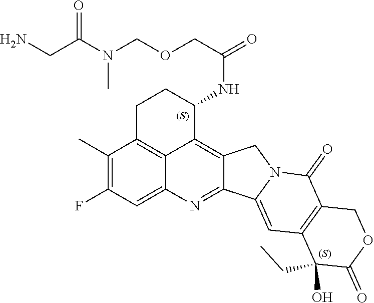 | −0.38 | C₃₀H₃₂FN₅O₇ | 593.61 | TFA | 98 | 594.2 |
| P11 | 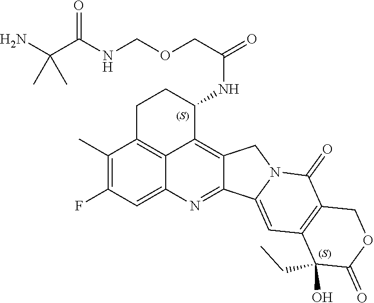 | 0.40 | C₃₁H₃₄FN₅O₇ | 607.64 | TFA | 100 | 607.6 |
| P12 | 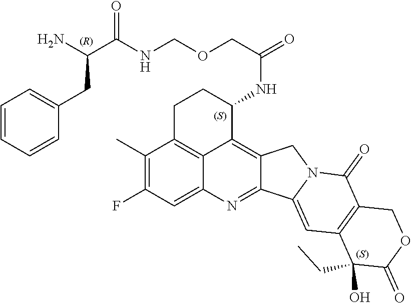 | 1.62 | C₃₆H₃₆FN₅O₇ | 669.71 | TFA | 98 | 670.2 |

Certain properties of the payloads according to the present disclosure are summarized in Table 2, below.

TABLE 2
SAR of ProDrugs of DXd (R1,R2,R3 = H)

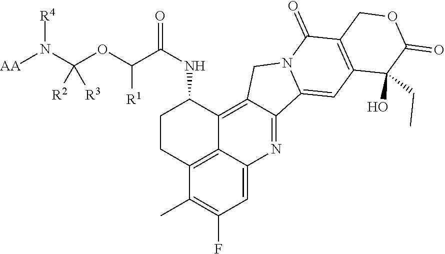

| | | | | | | | Human plasma | | | | |
|---|---|---|---|---|---|---|---|---|---|---|---|
| | | | | | | | stability | | Mouse blood stability | | |
| | | | | HCT-15 EC₅₀ | | SKBR | | % | | % | DXd |
| Cpd | Structure | | cLog | (nM) | | 3 EC₅₀ | T1/2 | Remaining | T1/2 | Remaining | released at |
| # | AA | R4 | P | −v | +v | (nM) | (h) | at 24 h | (h) | at 24 h | 24 h |
| EXT | | | 1.45 | 1.22 | 1.13 | 0.52 | | | | | |
| DXd | | | 0.55 | 7.99 | 4.18 | 1.63 | | | 62.65 | 94.8% | |
| P1 | Gly | H | −0.60 | 49.85 | 18.16 | 9.52 | 49.6 | 72.4 | 0.63 | 4.1% | 37.4% |
| P2 | Ser | H | −1.08 | 61.65 | 31.88 | 5.89 | 65.4 | 80.4 | 2.5 | 34.1% | 40.3% |
| P3 | Gln | H | −1.19 | 120.45 | 65.07 | 11.6 | 24.1 | 48.9 | 1.0 | 6.5% | 15.8% |
| P4 | Glu | H | −2.65 | 78.09 | 65.34 | 6.63 | 107 | 90.4 | 9.2 | 66.1% | 10.8% |
| P5 | Lys | H | −0.36 | 25.14 | 20.39 | 4.51 | 49.4 | 69.7 | 0.4 | 1.3% | 55.3% |
| P6 | Lys(N₃) | H | 0.76 | 7.74 | 3.18 | 2.06 | 28.2 | 59.0 | 0.2 | 3.3% | 37.3% |
| P7 | Phe | H | 1.62 | 12.68 | 4.55 | 1.98 | 24.4 | 51.3 | 0.2 | 6.3% | 40.1% |
| P8 | Gly-Gly | H | −1.71 | 142.12 | 125.46 | 30.82 | 144 | 86.0 | 2.9 | 46.9% | 21.8% |
| P9 | Gly | Me | −0.38 | 193.71 | 99.73 | 69.50 | | | | | |
| P11 | COCMe₂NH₂ | H | 0.40 | 166.47 | 66.82 | 27.68 | | | | | |
| P12 | D-Phe | H | 1.62 | 94.37 | 32.00 | 15.35 | | | | | |

The present disclosure also relates to a pharmaceutical composition comprising a therapeutically effective amount of the payload as described above or a pharmaceutically acceptable salt thereof and one or more pharmaceutically acceptable carriers, diluents, or excipients.

The present disclosure also relates to a process for manufacturing a linker-payload compound having the formula (D′)-(G′)

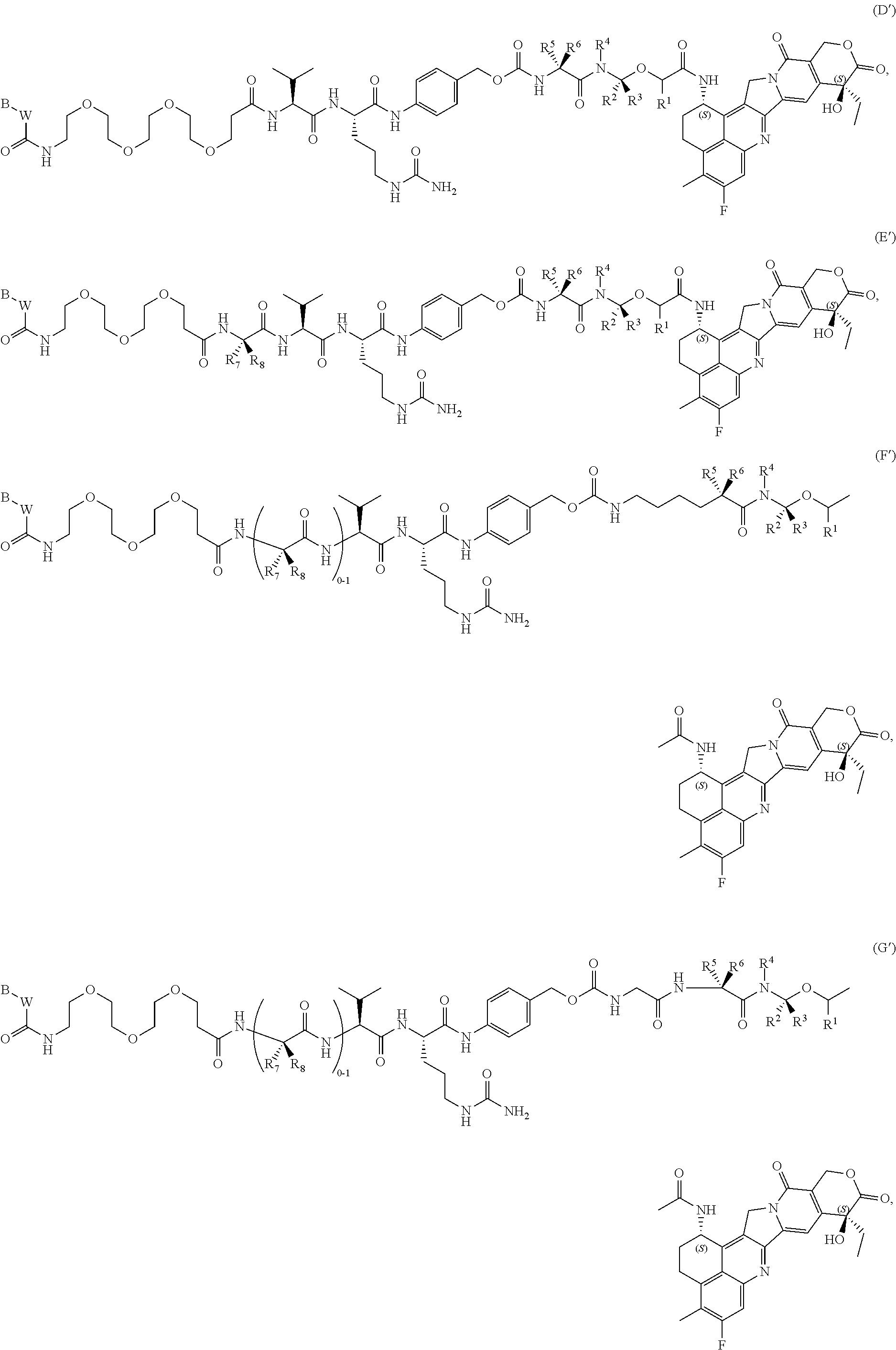

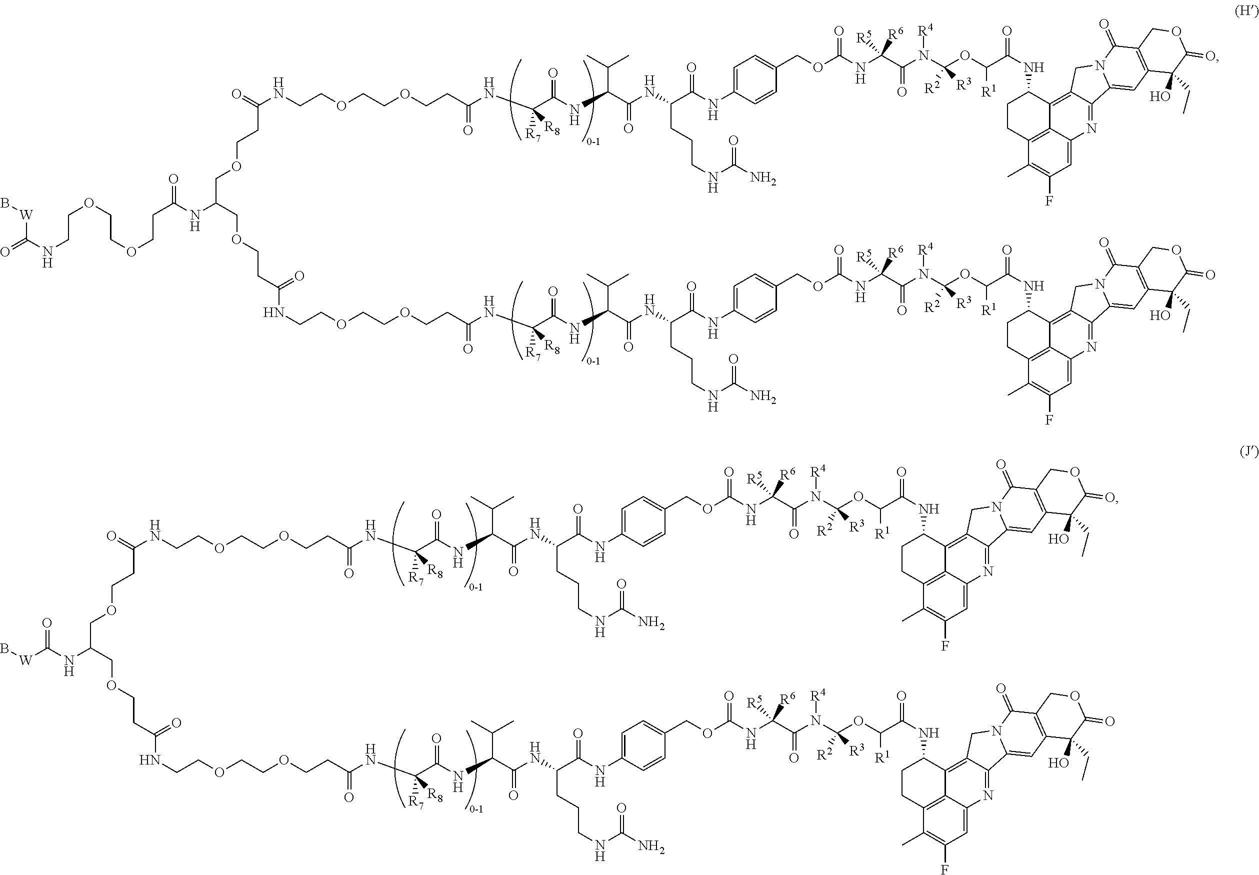

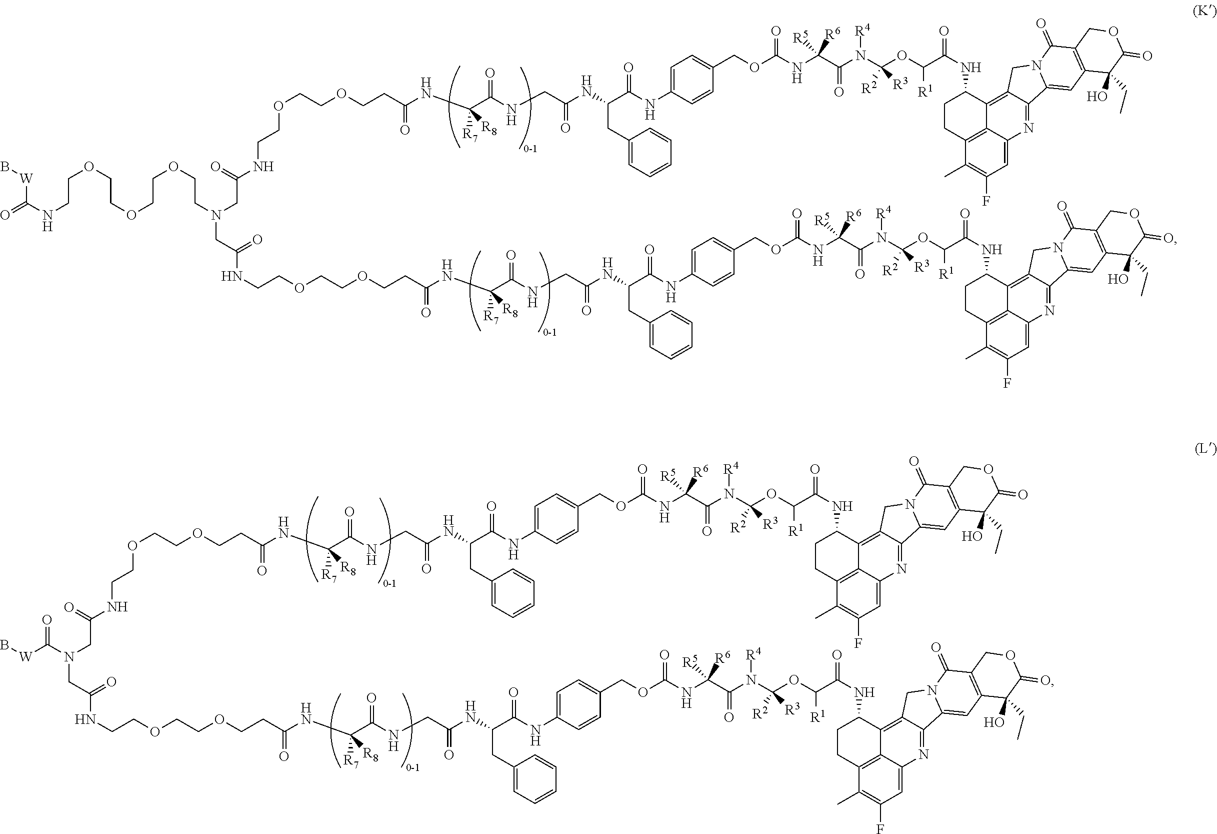

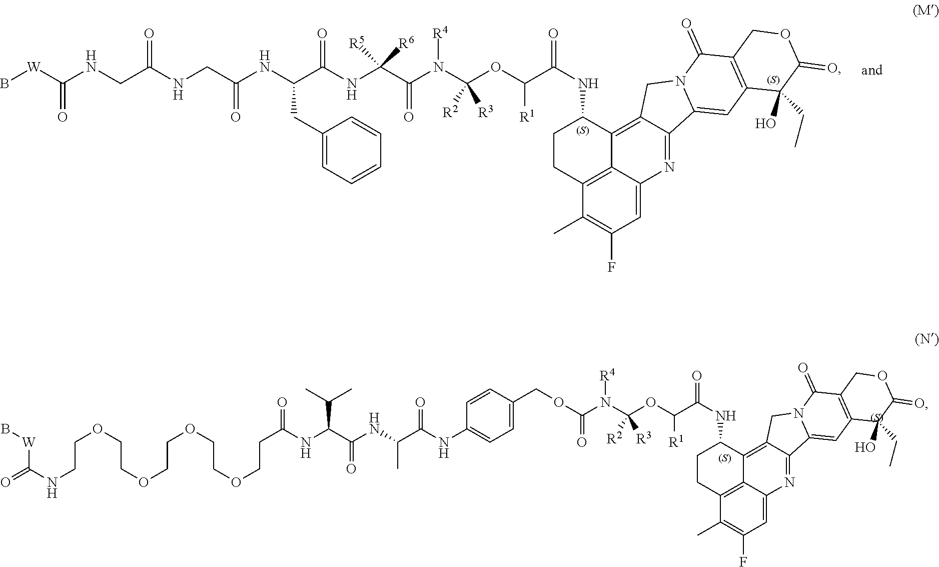

or a pharmaceutically acceptable salt thereof,
wherein R¹, R², R³, and R⁴ are independently hydrogen or a C₁₋₅ alkyl;
B is selected from the group consisting of

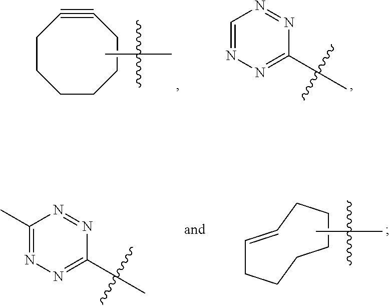

W is NH, O, CO, CH₂, a phenyl, or a combination of two or more thereof; and R⁵, R⁶, R⁷ and R⁸ are independently hydrogen, —NH₂, or a side chain of any natural or non-natural amino acid,
the method comprising a step of exposing a payload having an amino group to an activated intermediate having a para-nitro-phenyl carbonate in the presence of a base and a coupling catalyst to afford said linker-payload compound (D′)-(G′), wherein said coupling catalyst is 4-Hydroxy-2-methylquinoline (MeHYQ).

The present disclosure also relates to process for manufacturing a linker-payload compound having the formula (D-1)

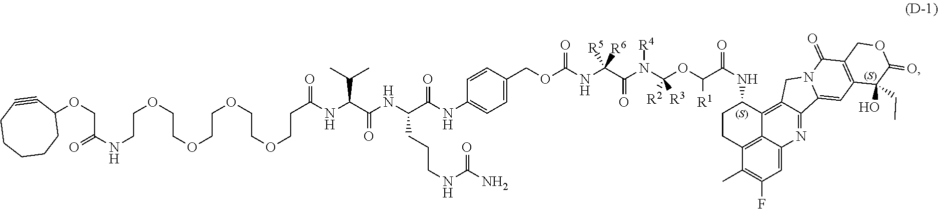

or a pharmaceutically acceptable salt thereof,
wherein R¹, R², R³, and R⁴ are independently hydrogen or a C₁₋₅ alkyl, and
R⁵ and R⁶ are independently hydrogen, —NH₂, or a side chain of any natural or non-natural amino acid,
the method comprising a step of exposing a payload having an amino group to an activated intermediate having a para-nitro-phenyl carbonate in the presence of a base and a coupling catalyst to afford said linker-payload compound (D-1), wherein said coupling catalyst is 4-Hydroxy-2-methylquinoline (MeHYQ).

In one embodiment, the payload having an amino group has a structure according to Formula P-I:

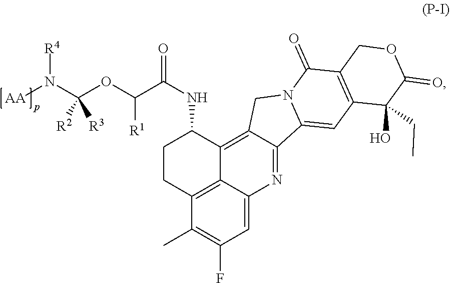

wherein R¹, R², R³, and R⁴ are independently hydrogen or a C₁₋₅ alkyl;
AA is a natural or a nonnatural amino acid; and
p is an integer from 1 to 6, or a pharmaceutically acceptable salt thereof.

In one embodiment, the amino group of the payload is the amino terminal of the AA.

In one embodiment, the activated intermediate having a para-nitro-phenyl carbonate has a structure according to formula I-I:

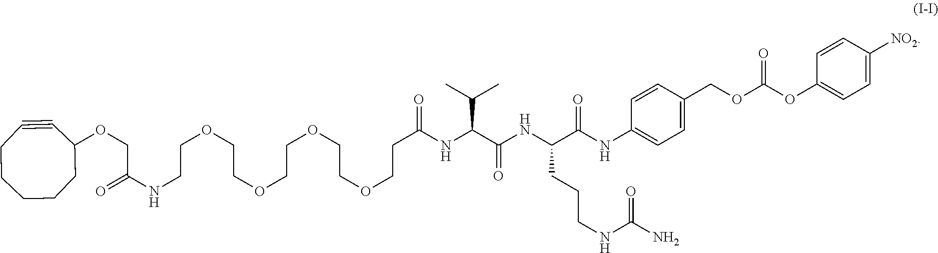 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(COC(=O)Oc2ccc([N+](=O)[O-])cc2)cc1]

The present disclosure also relates to a process for manufacturing a linker-payload compound having the formula (D-1)

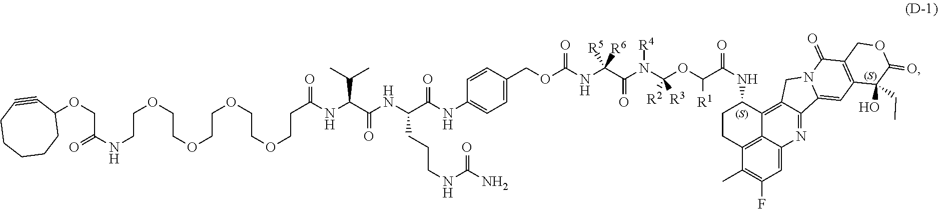

or a pharmaceutically acceptable salt thereof,
wherein R¹, R², R³, and R⁴ are independently hydrogen or a C₁₋₅ alkyl, and
R⁵ and R⁶ are independently hydrogen, —NH₂, or a side chain of any natural or non-natural amino acid,
said process comprising:
(a) providing a compound of Formula (1-1) having the structure:

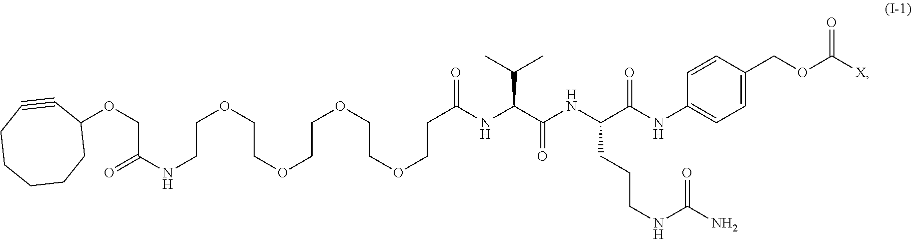

where
X is selected from the group consisting of

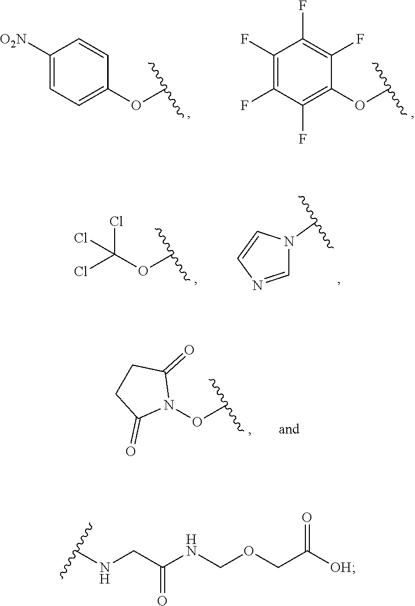 [SMILES: *Oc1ccc([N+](=O)[O-])cc1 ; *Oc1c(F)c(F)c(F)c(F)c1F ; *OC(Cl)(Cl)Cl ; *n1ccnc1 ; *ON1C(=O)CCC1=O ; *NCC(=O)NCOCC(=O)O]

and
(b) reacting the compound of Formula (1-1) with a compound of Formula (P-I):

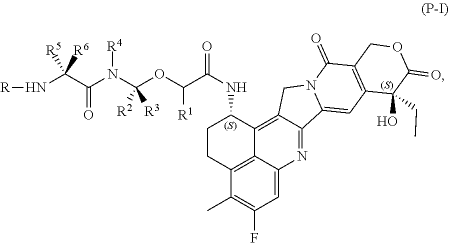

wherein
R is H or PG; and
PG is a suitable protecting group;
to produce the compound of Formula (D-1).

In one embodiment, the compound of Formula (D-1) has the following structure:

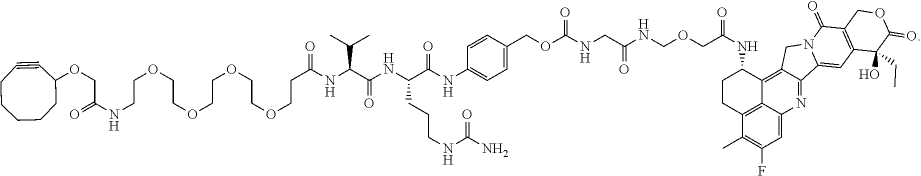 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

In one embodiment, the step (b) of reacting the compound of Formula (1-1) with the compound of Formula (P-I) further comprises reacting the compound of Formula (P-I), wherein R is PG, with a protecting group removing agent prior to said reacting with the compound of Formula (1-1).

In one embodiment, the PG is selected from the group consisting of allyloxycarbonyl (Alloc), benzyloxycarbonyl (Cbz), tert-butyloxycarbonyl (Boc), and 9-fluorenylmethoxycarbonyl (Fmoc).

In one embodiment, the protecting group removing agent is selected from the group consisting of Pd(PPh)₃, PhSiH₃, H₂, piperidine, and trifluoroacetic acid (TFA).

In one embodiment, the compound of Formula (1-1) has the following structure:

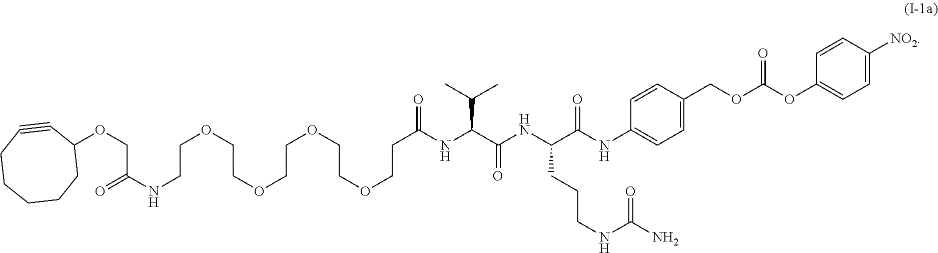 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(COC(=O)Oc2ccc([N+](=O)[O-])cc2)cc1]

In one embodiment, the compound of Formula (P-I) has the following structure:

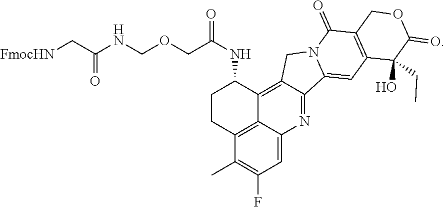

In one embodiment, the process for manufacturing a linker-payload compound having the formula (D-1) further comprises the steps of providing a compound of Formula (V) having the structure:

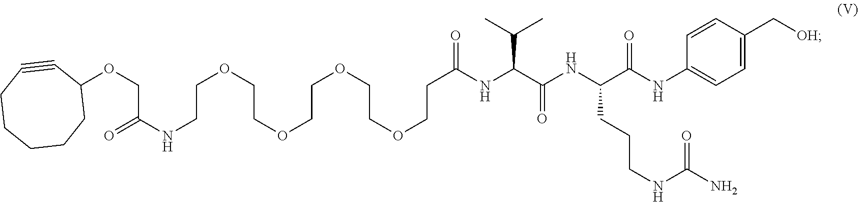 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(CO)cc1]

and forming the compound of Formula (1-1) from the compound of Formula (V) prior to the step (a).

In one embodiment, the step of forming the compound of Formula (1-1) comprises reacting the compound of Formula (V) with a compound of Formula (VIa) or Formula (VIb):

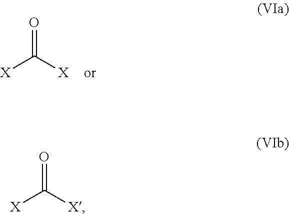

where X′ is halogen, to produce the compound of Formula (1-1).

In one embodiment, the compound of Formula (VIa) is selected from the group consisting of

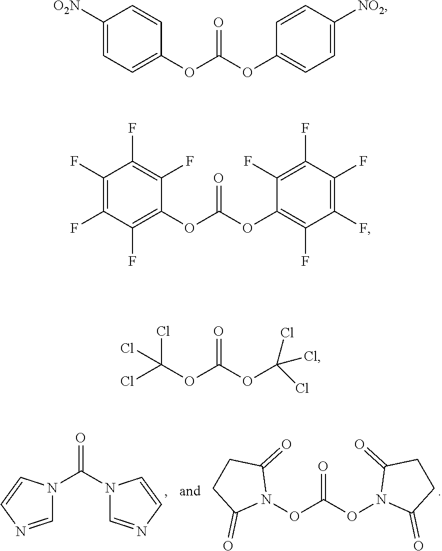 [SMILES: O=C(Oc1ccc([N+](=O)[O-])cc1)Oc1ccc([N+](=O)[O-])cc1 ; O=C(Oc1c(F)c(F)c(F)c(F)c1F)Oc1c(F)c(F)c(F)c(F)c1F ; O=C(OC(Cl)(Cl)Cl)OC(Cl)(Cl)Cl ; O=C(n1ccnc1)n1ccnc1 ; O=C(ON1C(=O)CCC1=O)ON1C(=O)CCC1=O]

In one embodiment, the compound of Formula (VIb) is

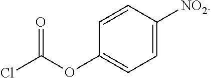 [SMILES: O=C(Cl)Oc1ccc([N+](=O)[O-])cc1]

In one embodiment, the process further comprises providing a compound of Formula (VII) having the structure:

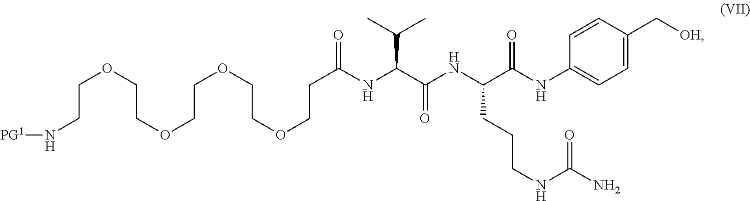

wherein PG¹ is a suitable protecting group protecting group, and forming the compound of Formula (V) from the compound of Formula (VII).

In one embodiment, the PG¹ is selected from the group consisting of allyloxycarbonyl (Alloc), benzyloxycarbonyl (Cbz), tert-butyloxycarbonyl (Boc), and 9-fluorenylmethoxycarbonyl (Fmoc).

In one embodiment, the compound of Formula (VII) has the following structure:

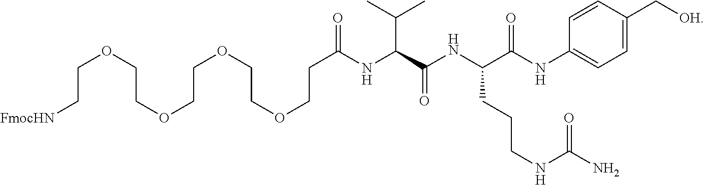

In one embodiment, the step of forming the compound of Formula (V) comprises reacting the compound of Formula (VII) with a compound of Formula (VIII):

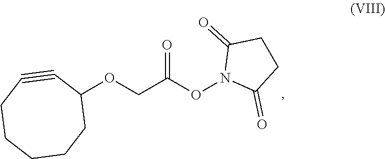 [SMILES: O=C(COC1C#CCCCCC1)ON1C(=O)CCC1=O]

to produce the compound of Formula (V).

In one embodiment, the process further comprising the steps of providing a compound of Formula (IX) having the structure:

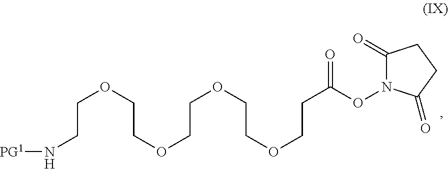

and forming the compound of Formula (VII) from the compound of Formula (IX).

In one embodiment, the compound of Formula (IX) has the following structure:

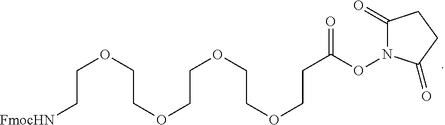

In one embodiment, the step of forming the compound of Formula (VII) comprises reacting the compound of Formula (IX) with a compound of Formula (X):

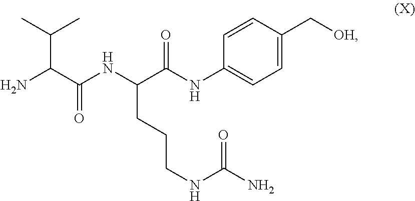 [SMILES: CC(C)C(N)C(=O)NC(CCCNC(N)=O)C(=O)Nc1ccc(CO)cc1]

to produce the compound of Formula (VII).

In one embodiment, the process further comprises the steps of providing a compound of Formula (XI) having the structure:

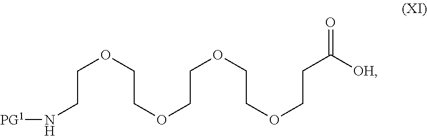

and forming the compound of Formula (IX) from the compound of Formula (XI).

In one embodiment, the compound of Formula (XI) has the following structure:

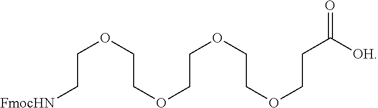

In one embodiment, the step of forming the compound of Formula (IX) comprises reacting the compound of Formula (XI) with a compound of Formula (XII):

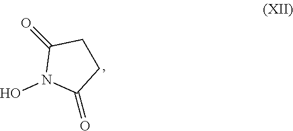

to produce the compound of Formula (IX).

In one embodiment, the process further comprises providing a compound of Formula (XIII) having the structure:

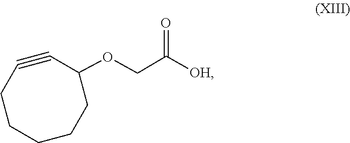 [SMILES: O=C(O)COC1C#CCCCCC1]

and
forming the compound of Formula (VIII) from the compound of Formula (XIII).

In one embodiment, the step of forming the compound of Formula (VIII) comprises reacting the compound of Formula (XIII) with a compound of Formula (XII):

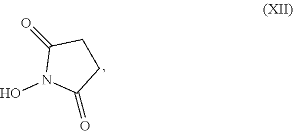

to produce the compound of Formula (VIII).

In one embodiment, the process further comprises the steps of providing a compound of Formula (XIV) having the structure:

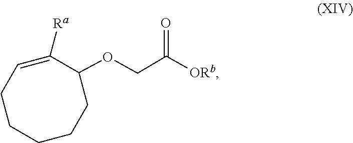

wherein R^{a} is halogen;
R^{b} is C₁₋₆ alkyl; and forming the compound of Formula (XIII) from the compound of Formula (XIV).

In one embodiment, R^{a} is bromine.

In one embodiment, the compound of Formula (XIV) has the following structure:

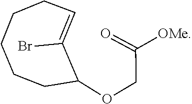 [SMILES: COC(=O)COC1CCCCC/C=C/1Br]

In one embodiment, the step of forming the compound of Formula (XIII) comprises reacting the compound of Formula (XIV) with a base to produce the compound of Formula (XIII).

In one embodiment, the base is selected from the group consisting of sodium methoxide (NaOMe), potassium tert-butoxide (t-BuOK), sodium hydride (NaH), and lithium diisopropylamide (LDA)

In one embodiment, the reaction between the compound of Formula (XIV) and a base is carried out in a suitable solvent, such as methanol (MeOH), tetrahydrofuran (THF), dimethylformamide (DMF), or a mixture thereof.

In one embodiment, the process further comprises the steps of providing a compound of Formula (XV) having the structure:

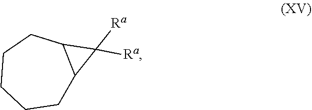

and
forming the compound of Formula (XIV) from the compound of Formula (XV).

In one embodiment, the compound of Formula (XV) has the following structure:

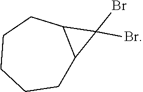

In one embodiment, the step of forming the compound of Formula (XIV) comprises reacting the compound of Formula (XV) with a compound of Formula (XVI):

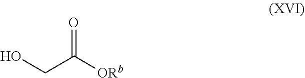

to produce the compound of Formula (XIV).

In one embodiment, the compound of Formula (XV) is reacted with methyl glycolate in the presence of AgOTf to produce the compound of Formula (XIV).

In one embodiment, the process further comprises the steps of providing a compound of Formula (XVII) having the structure:

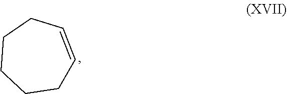

and forming the compound of Formula (XV) from the compound of Formula (XVII).

In one embodiment, the step of forming the compound of Formula (XV) comprises reacting the compound of Formula (XVII) with a bromination agent to produce the compound of Formula (XVII).

In one embodiment, the bromination agent is CHBr₃.

In one embodiment, the compound of Formula (XVII) is reacted with CHBr₃ in a non-polar solvent in the presence of a base, such as potassium tert-butoxide (t-BuOK).

In one embodiment, the process further comprises the steps of providing a compound of Formula (XVIII) having the structure:

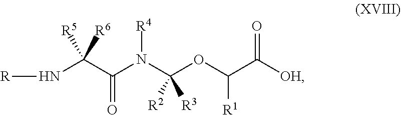

and forming the compound of Formula (P-I) from the compound of Formula (XVIII).

In one embodiment, the compound of Formula (XVIII) has the following structure:

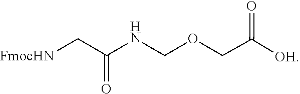

In one embodiment, the step of forming the compound of Formula (P-I) comprises reacting the compound of Formula (XVIII) with a compound of Formula (XIX):

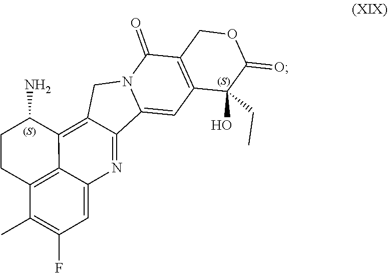

to produce the compound of Formula (P-I).

In one embodiment, the process further comprises the steps of providing a compound of Formula (XX) having the structure:

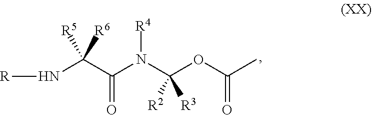

and forming the compound of Formula (XVIII) from the compound of Formula (XX).

In one embodiment, the compound of Formula (XX) has the following structure:

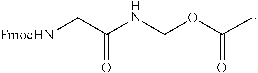

In one embodiment, the step of forming the compound of Formula (XVIII) comprises reacting the compound of Formula (XX) with a compound of Formula (XXI):

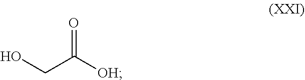

to produce the compound of Formula (XVIII).

In one embodiment, the process further comprises the steps of providing a compound of Formula (XXII) having the structure:

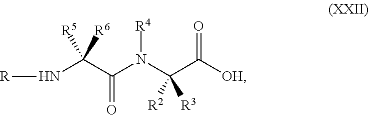

and forming the compound of Formula (XX) from the compound of Formula (XXII).

In one embodiment, the compound of Formula (XXII) has the following structure:

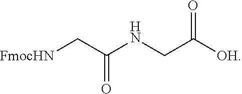

The present disclosure also relates to a process for preparation of a compound of Formula (1-1):

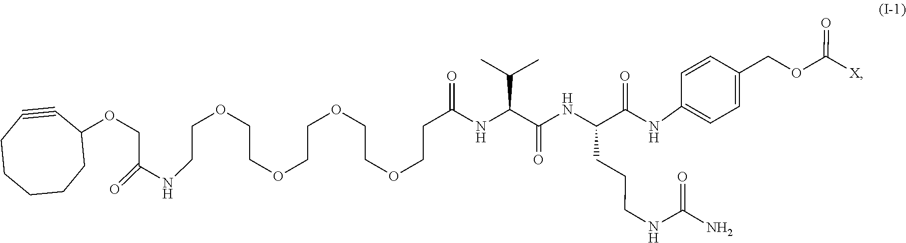

or a pharmaceutically acceptable salt thereof,
where
X is selected from the group consisting of

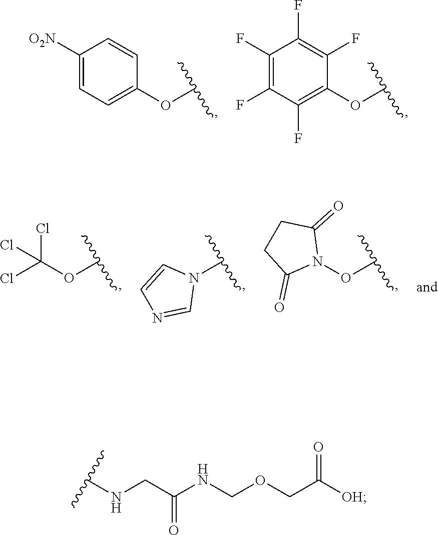

said process comprising:
(a) providing a compound of Formula (V) having the structure:

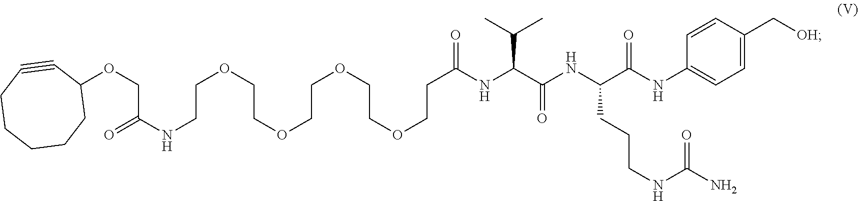 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(CO)cc1]

and
(b) forming the compound of Formula (1-1) from the compound of Formula (V).

In one embodiment, the compound of Formula (1-1) has the following structure:

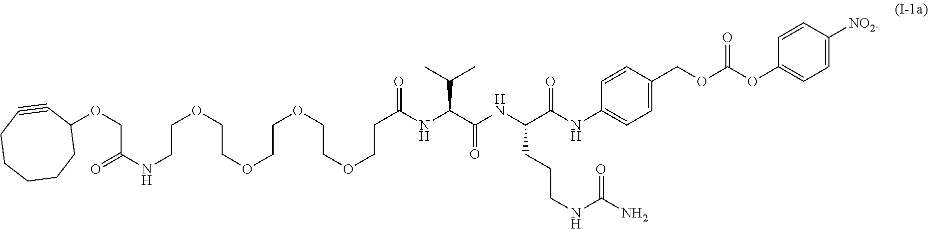 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(COC(=O)Oc2ccc([N+](=O)[O-])cc2)cc1]

In one embodiment, step (b) of forming the compound of Formula (1-1) comprises reacting the compound of Formula (V) with a compound of Formula (VIa) or Formula (VIb):

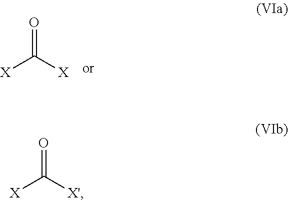

where X′ is halogen,
to produce the compound of Formula (1-1).

In one embodiment, the compound of Formula (VIa) is selected from the group

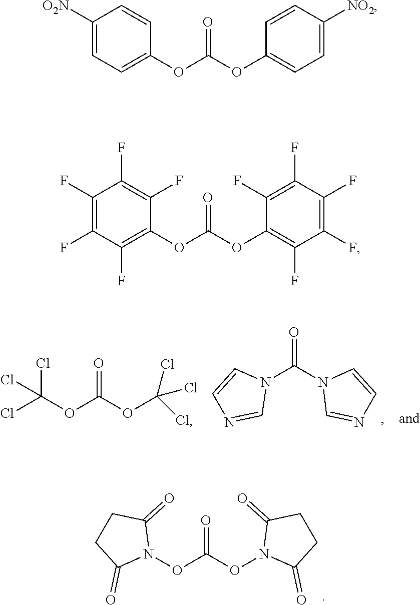

In one embodiment, the compound of Formula (VIb) is

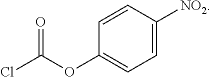 [SMILES: O=C(Cl)Oc1ccc([N+](=O)[O-])cc1]

In one embodiment, the process further comprises the steps of providing a compound of Formula (VII) having the structure:

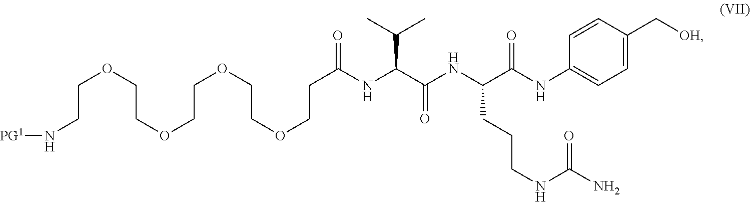

wherein PG¹ is a suitable protecting group protecting group, and
forming the compound of Formula (V) from the compound of Formula (VII).

In one embodiment, the compound of Formula (VII) has the following structure:

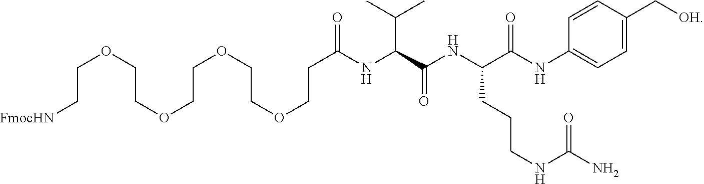

In one embodiment, the step of forming the compound of Formula (V) comprises reacting the compound of Formula (VII) with a compound of Formula (VIII):

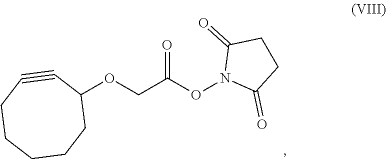 [SMILES: O=C(COC1C#CCCCCC1)ON1C(=O)CCC1=O]

to produce the compound of Formula (V).

In one embodiment, the process further comprises the steps of providing a compound of Formula (IX) having the structure:

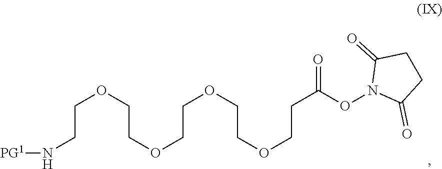

and forming the compound of Formula (VII) from the compound of Formula (IX).

In one embodiment, the compound of Formula (IX) has the following structure:

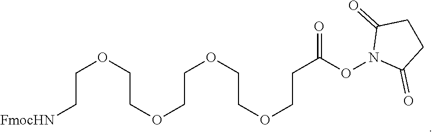

In one embodiment, the step of forming the compound of Formula (VII) comprises reacting the compound of Formula (IX) with a compound of Formula (X):

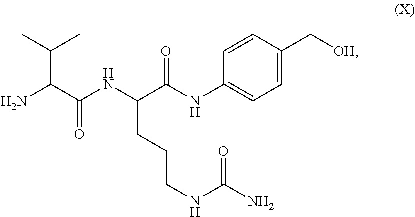 [SMILES: CC(C)C(N)C(=O)NC(CCCNC(N)=O)C(=O)Nc1ccc(CO)cc1]

to produce the compound of Formula (VII).

In one embodiment, the process further comprises the steps of providing a compound of Formula (XI) having the structure:

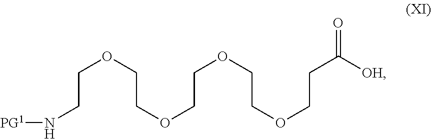

and forming the compound of Formula (IX) from the compound of Formula (XI).

In one embodiment, the compound of Formula (XI) has the following structure:

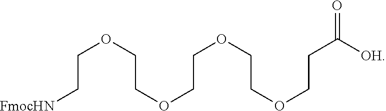

In one embodiment, the step of forming the compound of Formula (IX) comprises reacting the compound of Formula (XI) with a compound of Formula (XII):

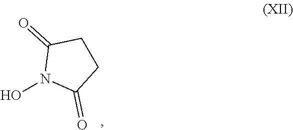

to produce the compound of Formula (IX).

The present disclosure also relates to a process for preparation of a compound of Formula (XVIII):

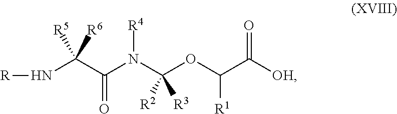

or a pharmaceutically acceptable salt thereof,
wherein R¹, R², R³, and R⁴ are independently hydrogen or a C₁₋₅ alkyl, and
R⁵ and R⁶ are independently hydrogen, —NH₂, or a side chain of any natural or non-natural amino acid. This process comprises:
(a) providing a compound of Formula (XX) having the structure:

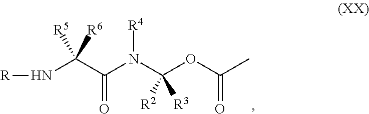

and
(b) forming the compound of Formula (XVIII) from the compound of Formula (XX).

In one embodiment, the compound of Formula (XVIII) has the following structure:

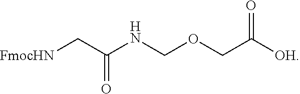

In one embodiment, the compound of Formula (XX) has the following structure:

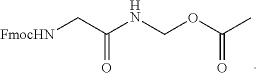

In one embodiment, the step of forming the compound of Formula (XVIII) comprises reacting the compound of Formula (XX) with a compound of Formula (XXI):

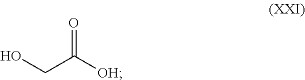

to produce the compound of Formula (XVIII).

In one embodiment, the process further comprises the steps of providing a compound of Formula (XXII) having the structure:

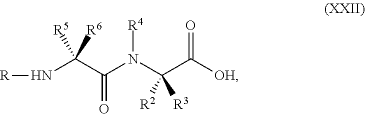

and forming the compound of Formula (XX) from the compound of Formula (XXII).

In one embodiment, the compound of Formula (XXII) has the following structure:

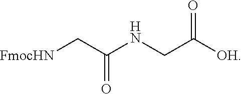

The present disclosure also relates to a process for preparation of a compound of Formula (D-1):

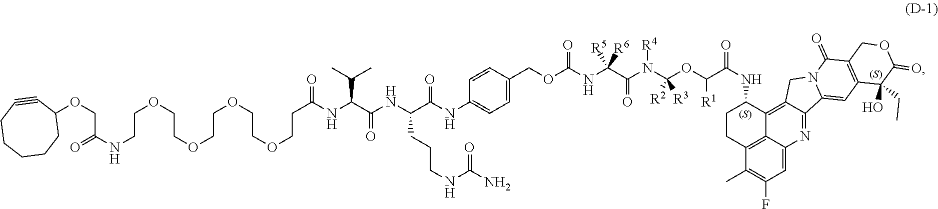

or a pharmaceutically acceptable salt thereof,
wherein R¹, R², R³, and R⁴ are independently hydrogen or a C₁₋₅ alkyl, and
R⁵ and R⁶ are independently hydrogen, —NH₂, or a side chain of any natural or non-natural amino acid. This process comprises:
(a) providing a compound of Formula (1-1) having the structure:

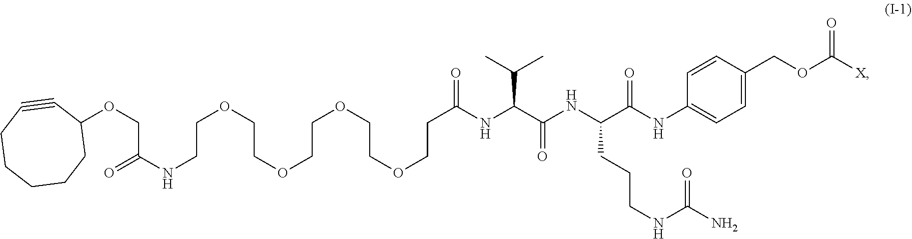

wherein
X is selected from the group consisting of

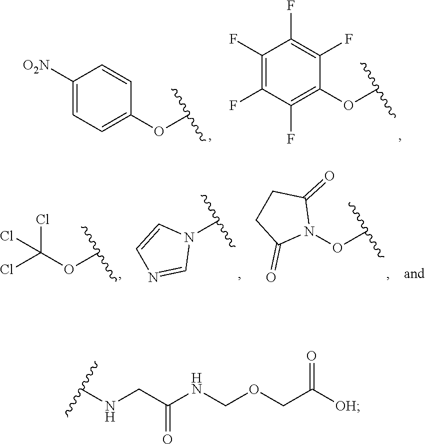

and
(b) reacting the compound of Formula (1-1) with a compound of Formula (P-I):

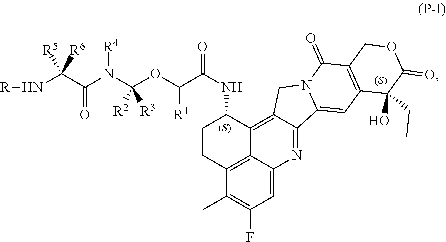

wherein
R is H or PG; and
PG is a suitable protecting group,
to produce the compound of Formula (D-1).

In one embodiment, the compound of Formula (D-1) has the following structure:

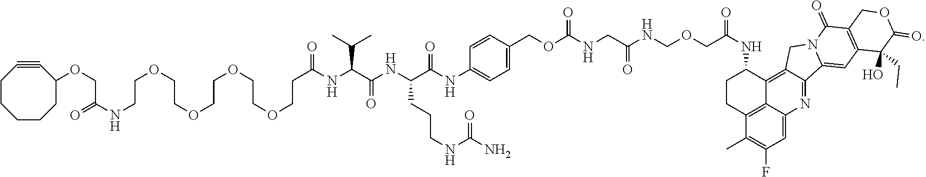 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

In one embodiment, the compound of Formula (1-1) has the following structure:

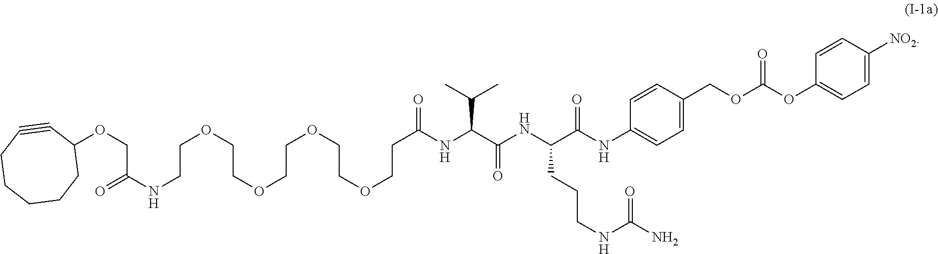 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(COC(=O)Oc2ccc([N+](=O)[O-])cc2)cc1]

In one embodiment, the step (b) of reacting the compound of Formula (1-1) with the compound of Formula (P-I) further comprises reacting the compound of Formula (P-I), wherein R is PG, with a protecting group removing agent prior to said reacting with the compound of Formula (1-1).

In one embodiment, the PG is selected from the group consisting of allyloxycarbonyl (Alloc), benzyloxycarbonyl (Cbz), tert-butyloxycarbonyl (Boc), and 9-fluorenylmethoxycarbonyl (Fmoc).

In one embodiment, the protecting group removing agent is selected from the group consisting of Pd(PPh)₃, PhSiH₃, H₂, piperidine, and trifluoroacetic acid (TFA).

In one embodiment, the compound of Formula (P-I) has the following structure:

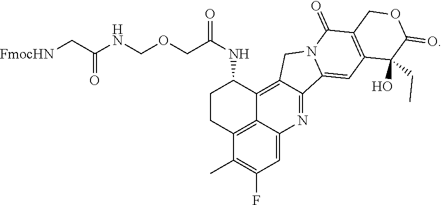

In one embodiment, the process further comprises the steps of providing a compound of Formula (XVIII) having the structure:

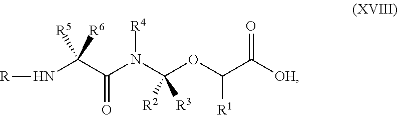

and forming the compound of Formula (P-I) from the compound of Formula (XVIII).

In one embodiment, the compound of Formula (XVIII) has the following structure:

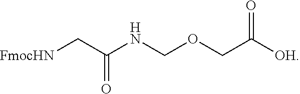

In one embodiment, the step of forming the compound of Formula (P-I) comprises reacting the compound of Formula (XVIII) with a compound of Formula (XIX):

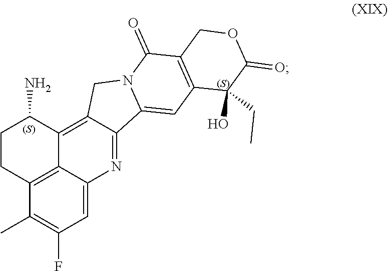

to produce the compound of Formula (P-I).

The present disclosure also relates to a process for preparation of a compound of Formula (D-1):

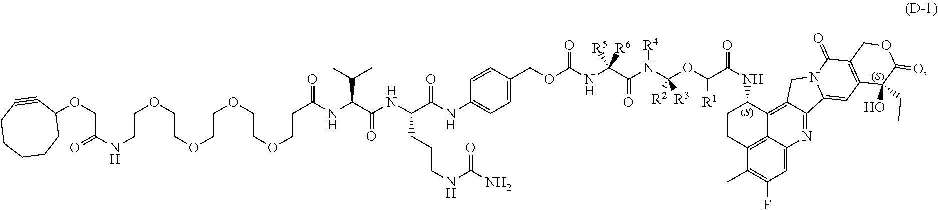

or a pharmaceutically acceptable salt thereof,
wherein R¹, R², R³, and R⁴ are independently hydrogen or a C₁₋₅ alkyl, and
R⁵ and R⁶ are independently hydrogen, —NH₂, or a side chain of any natural or non-natural amino acid,
said process comprising:
(a) providing a compound of Formula (XXIII):

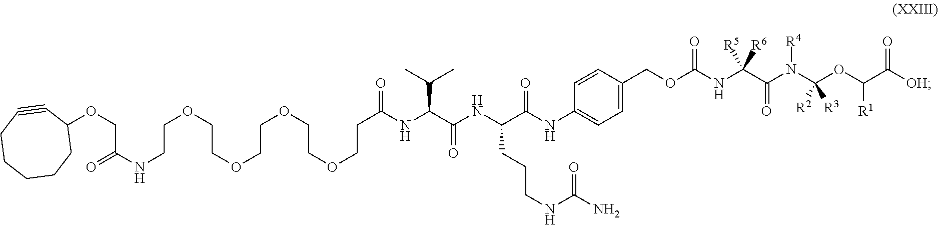

and
(b) reacting the compound of Formula (XXIII) with a compound having the structure:

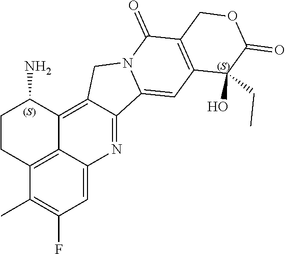

in the presence of an activating reagent and a base to produce the compound of Formula (D-1).

In one embodiment, the compound of Formula (D-1) has the following structure:

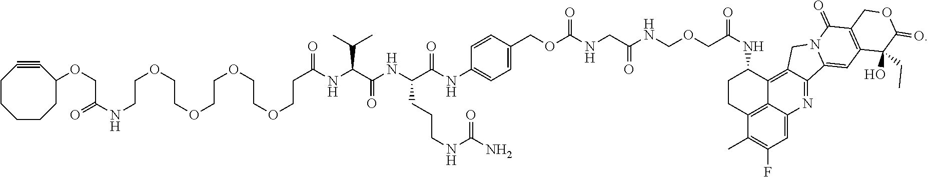 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

In one aspect, the present disclosure provides a linker-payload compound of formulas (D)-(G),

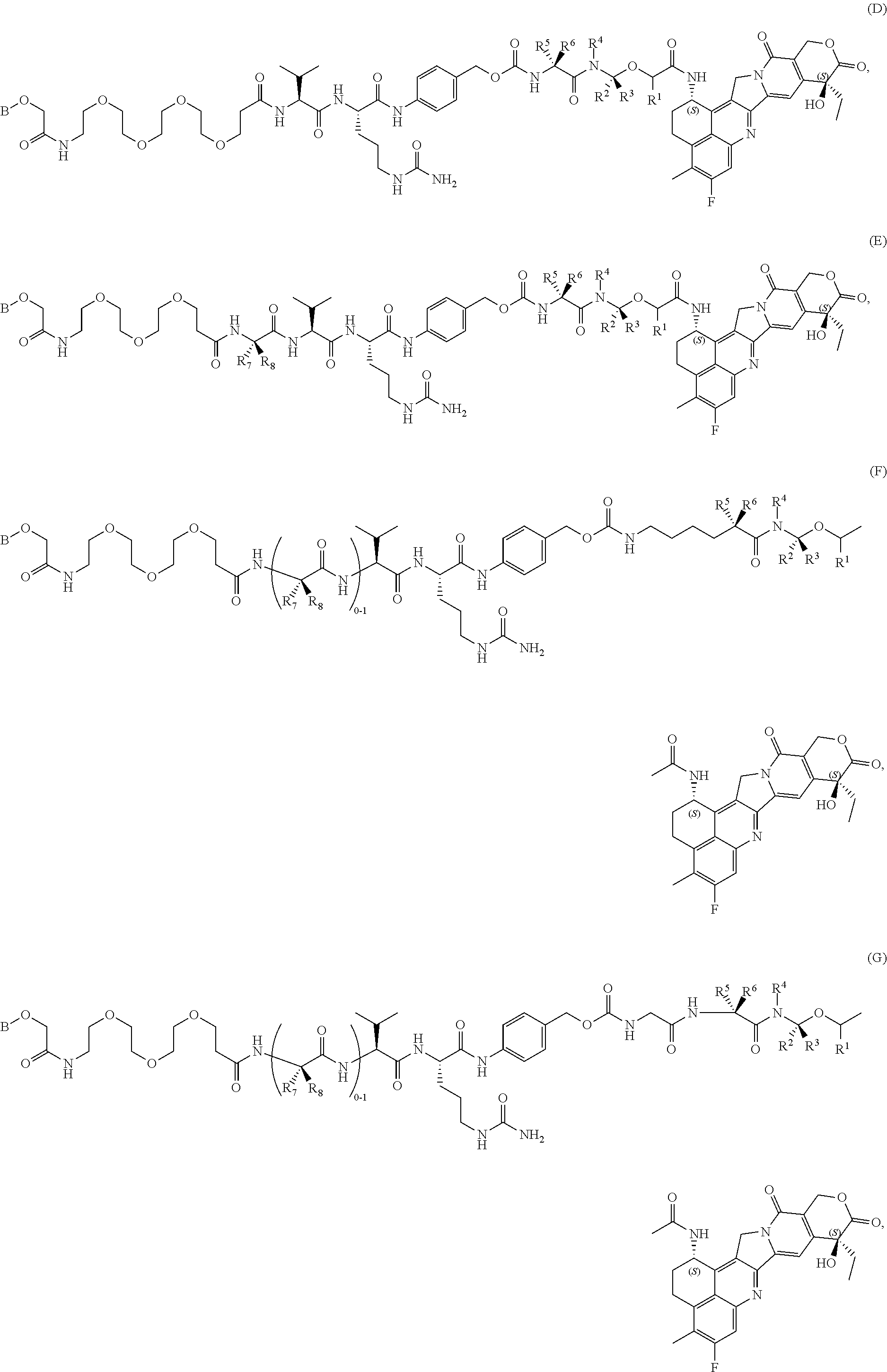

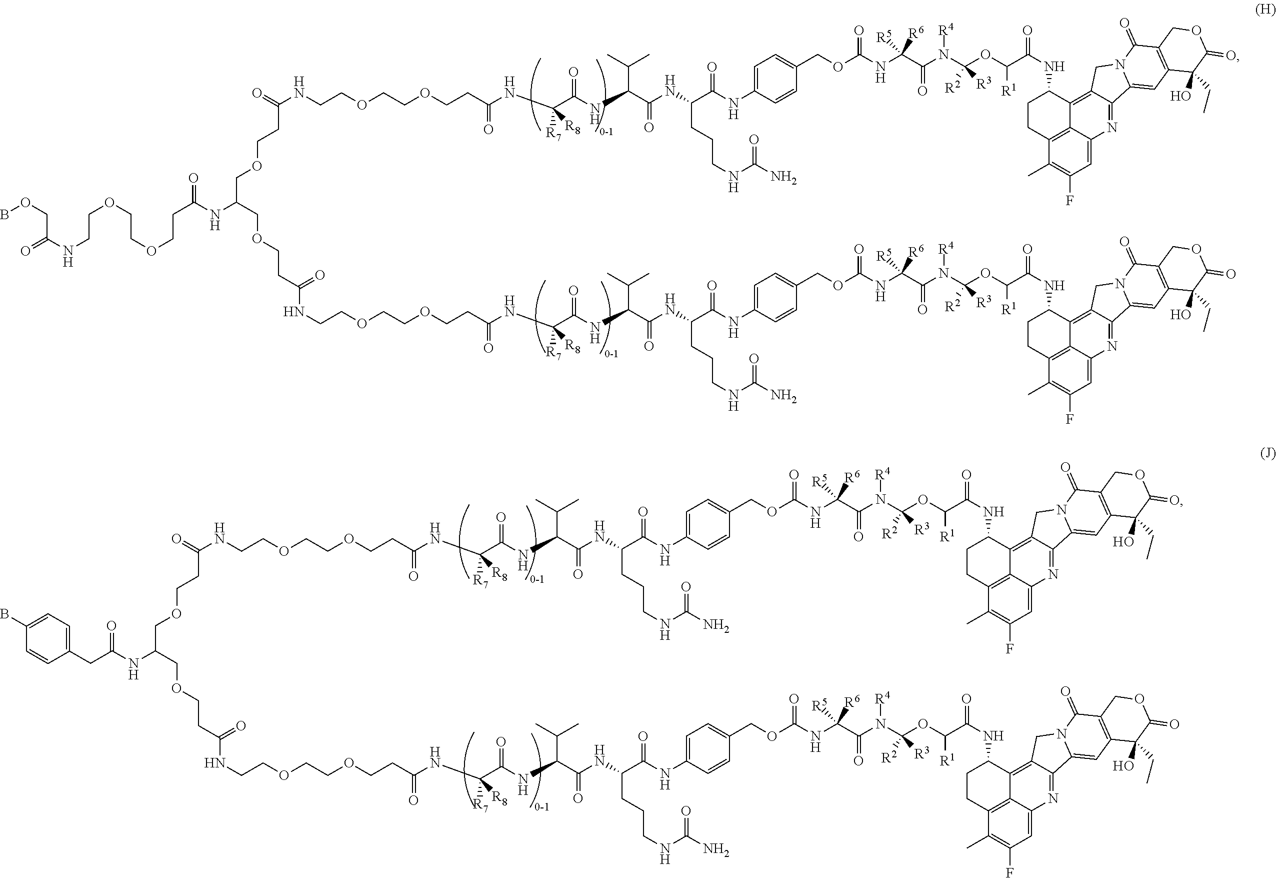

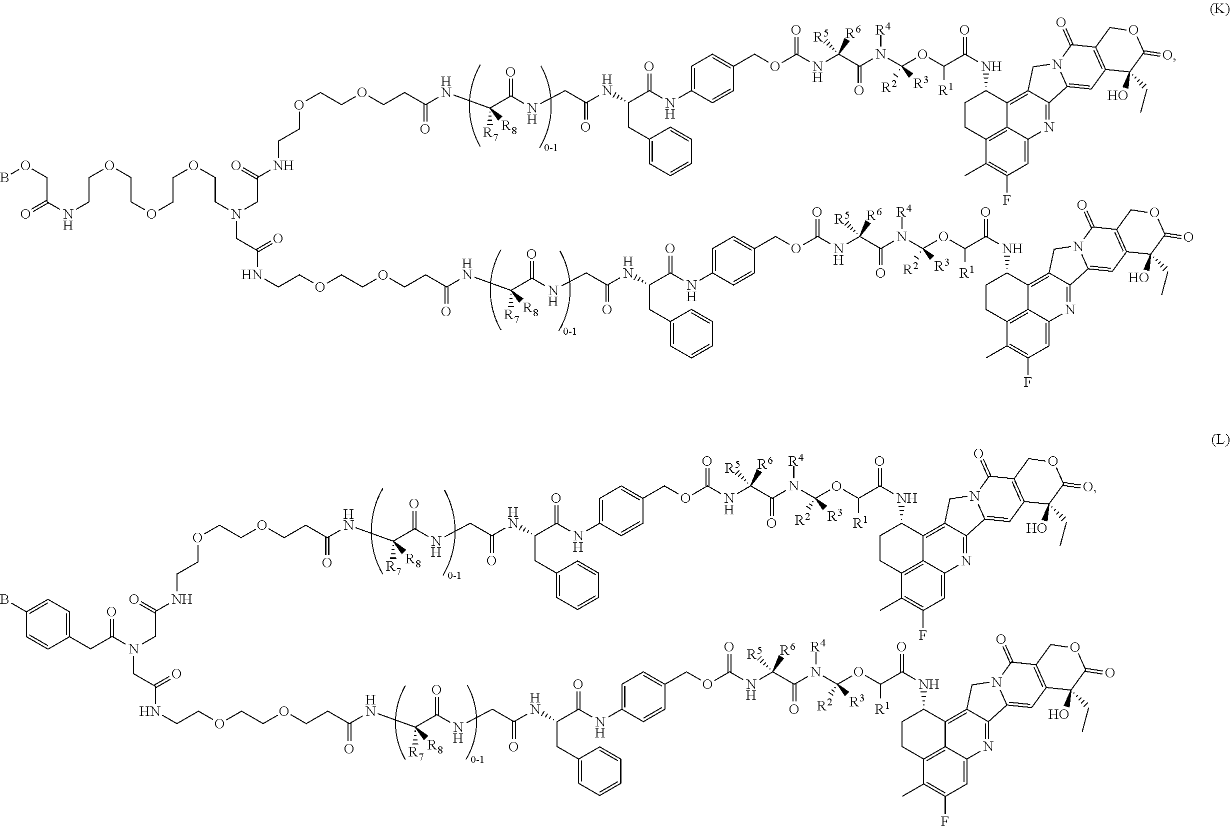

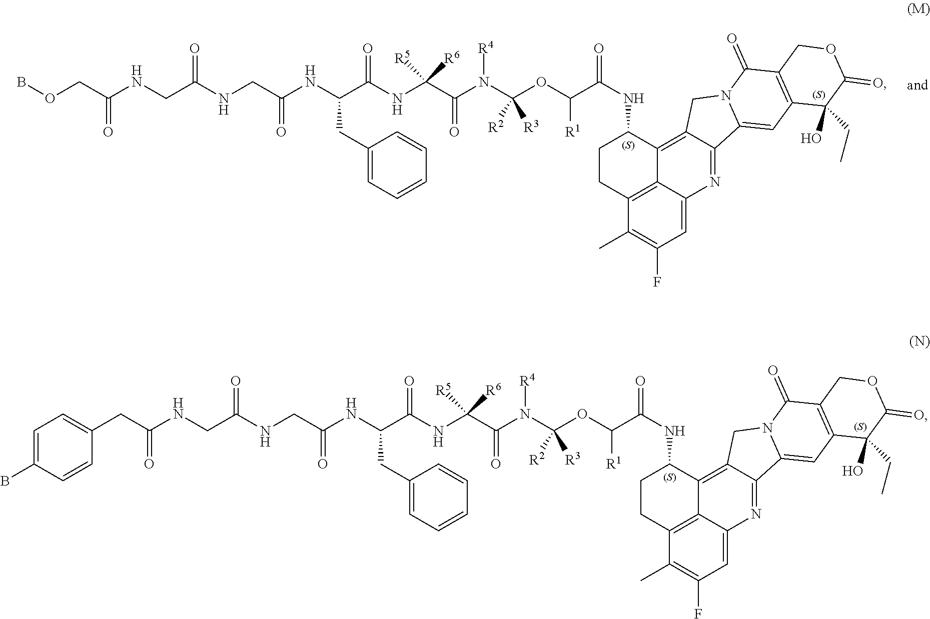

or a pharmaceutically acceptable salt thereof,
wherein B is selected from the group consisting of and

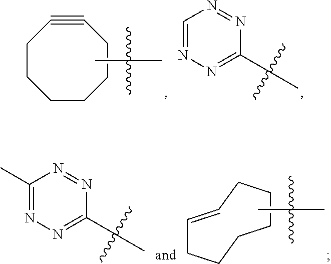

R¹, R², R³, and R⁴ are independently hydrogen or a C₁₋₅ alkyl, and
R⁵, R⁶, R⁷ and R⁸ are independently hydrogen, —NH₂, or a side chain of any natural or nonnatural amino acid.

In one embodiment, R¹, R², R³, and R⁴ are each hydrogens.

In one embodiment, R⁶ is H.

In one embodiment, R⁵ is selected from hydrogen and a side chain of alanine, valine, leucine, isoleucine, phenylalanine, tyrosine, threonine, lysine, asparagine, glutamine, aspartic acid, and glutamic acid. In one embodiment, R⁵ is selected from hydrogen and a side chain of phenylalanine, threonine, lysine, glutamine, and glutamic acid.

In one embodiment, R⁷ is H. In one embodiment, R⁷ is a side chain of glutamic acid.

In one embodiment, R⁸ is H. In one embodiment, R⁸ is —CH₂—SO₃H.

In one embodiment, the present disclosure provides a linker-payload having a structure selected from the group of Table 3, below.

TABLE 3
Structures of Linker-ProDXds

| | ProDXd | |
|---|---|---|
| | payload | |
| Cpd# | # | Structures |
| LP1 | P1 CP1190 | 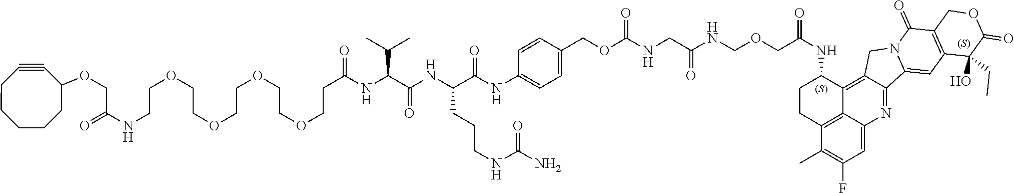 |
| LP1A | P1 CP1190 | 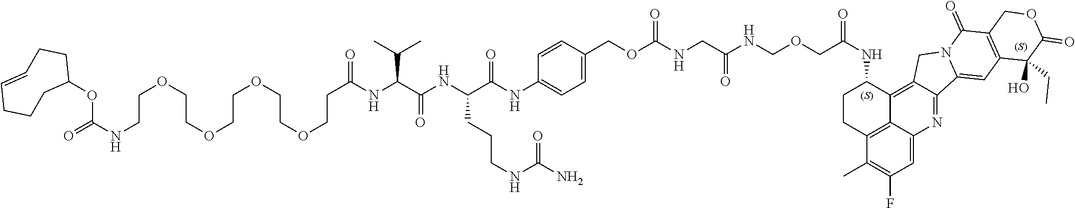 |
| LP1B | P1 CP1190 | 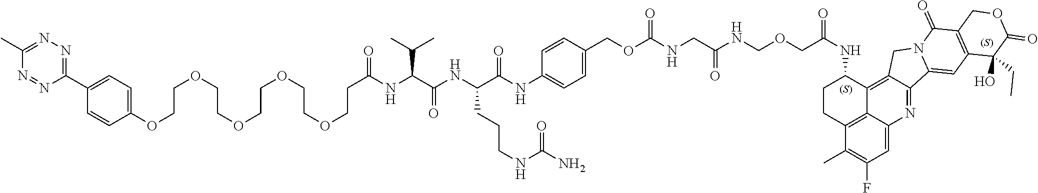 |
| LP1D | P1 CP1190 | 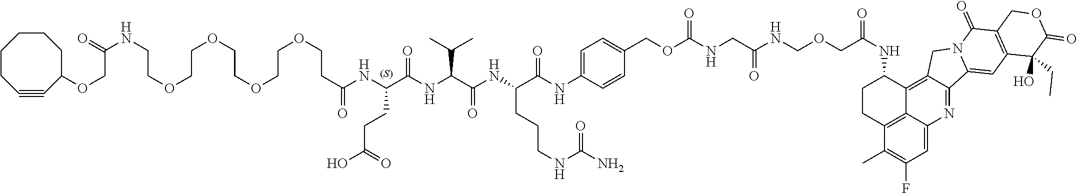 |
| LP1E | P1 CP1190 | 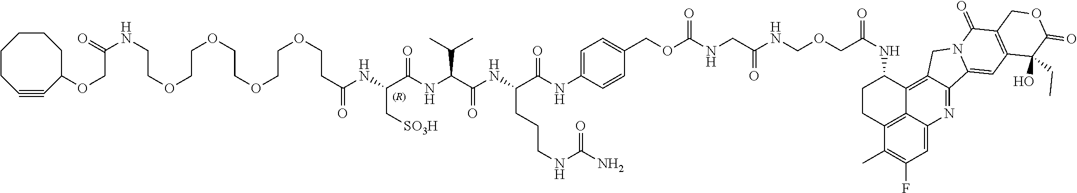 |
| LP2 | P2 CP1412 | 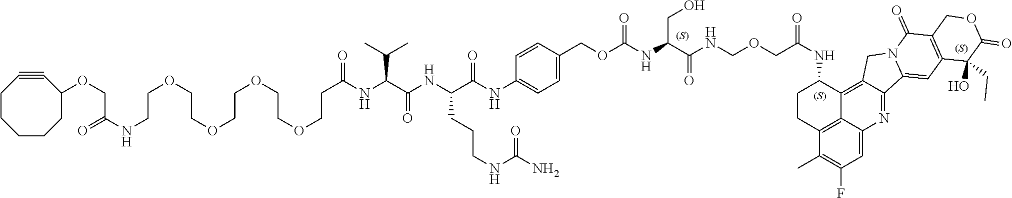 |
| LP3 | P3 CP1408 | 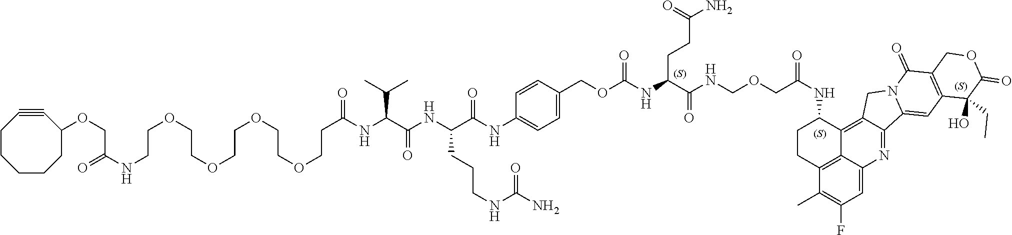 |
| LP4 | P4 CP1409 | 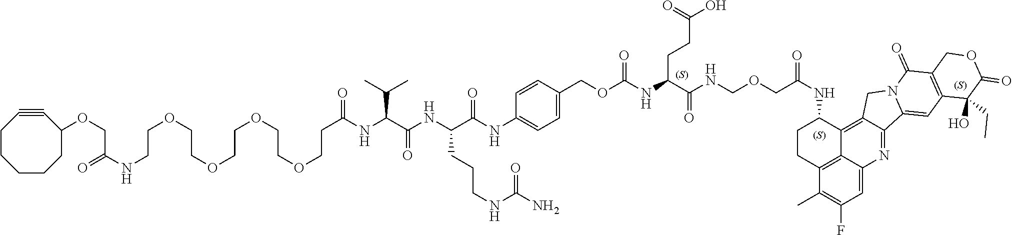 |
| LP5 | P5 CP1410 | 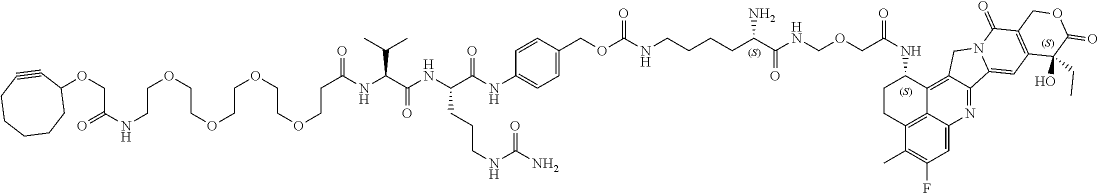 |
| LP5C | P5 CP1410 | 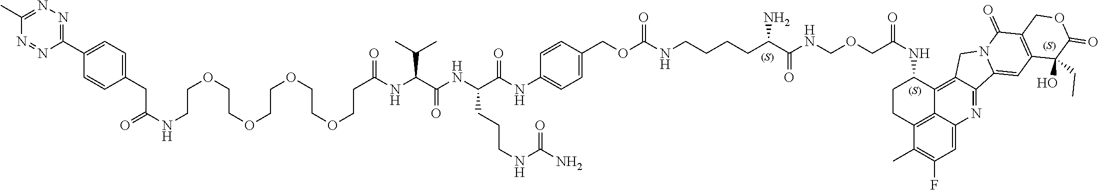 |
| LP5D | P5 CP1410 | 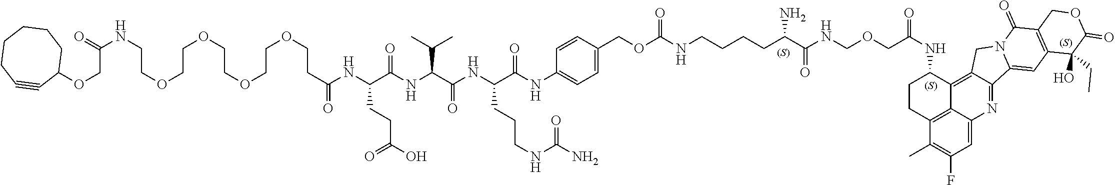 |
| LP7 | P7 CP1411 | 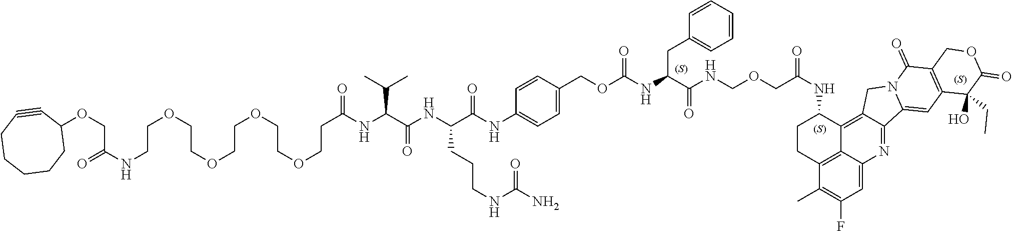 |
| LP8 | P8 CP1396 | 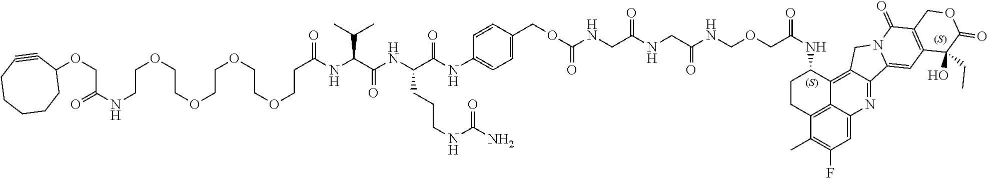 |
| LP9 | P9 CP1422 | 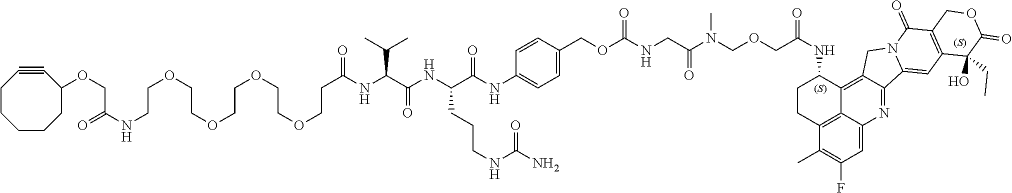 |
| LP10 | P11 CP1429 | 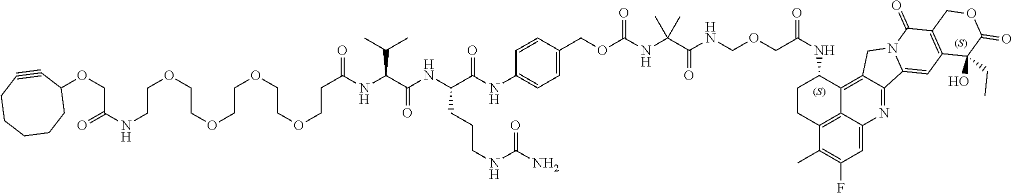 |
| LP11 | P12 CP1436 | 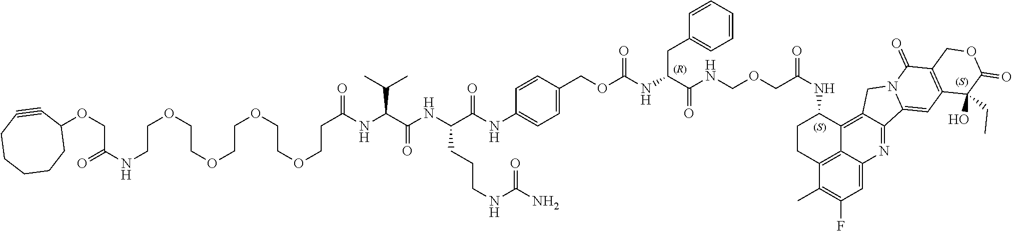 |
| LP12 | P5 CP1410 | 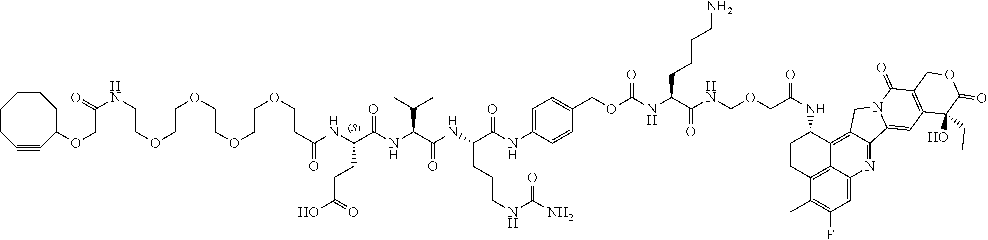 |
| LP13 | P1 CP1190 | 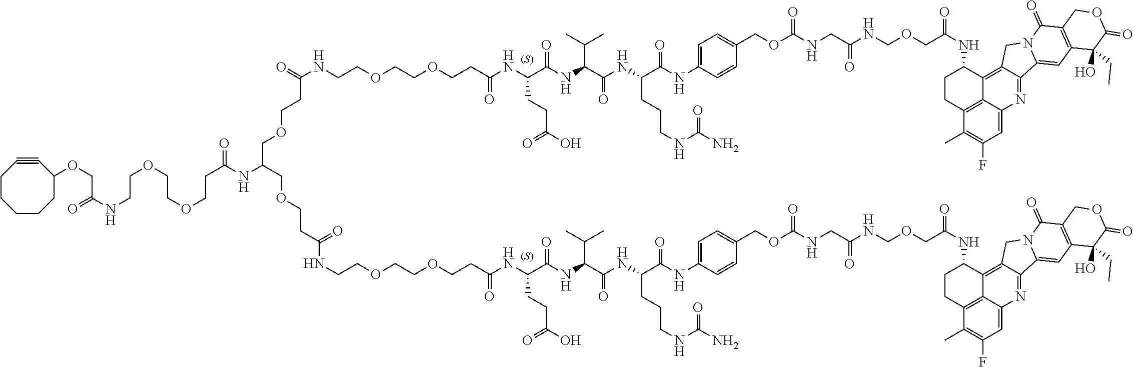 |
| LP13C | P1 | 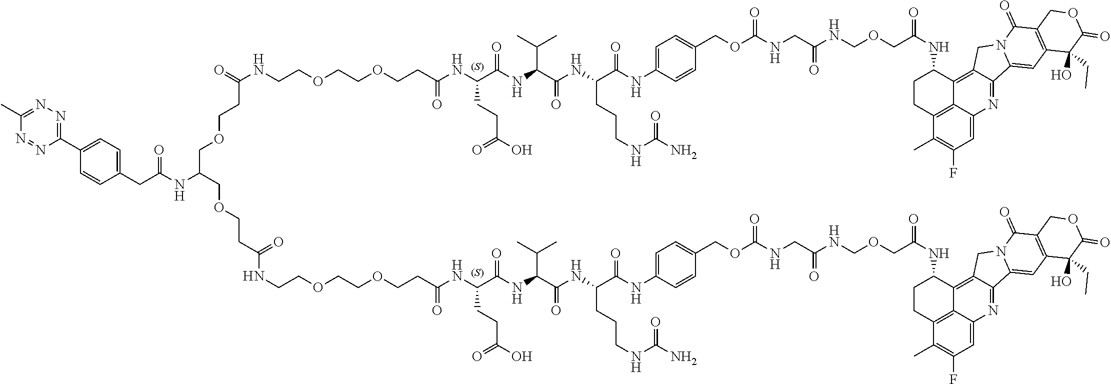 |
| LP14 | DXd | 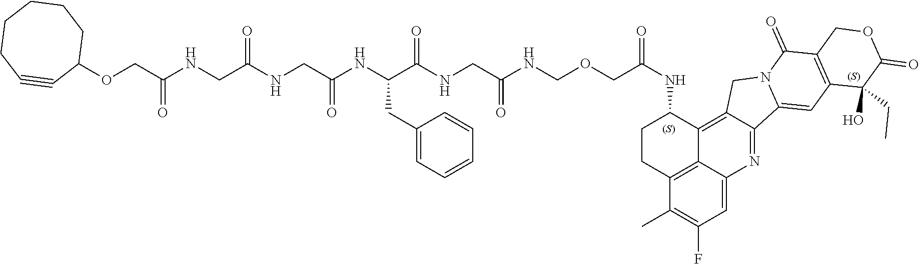 |
| | | (SEQ ID NO: 2117) |
| LP14C | DXd | 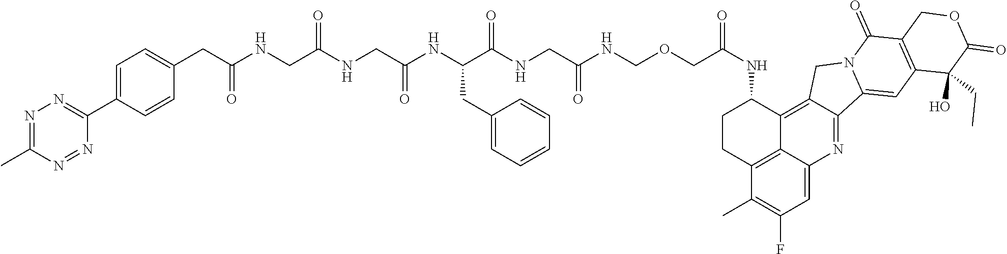 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)Cc1ccc(-c2nnc(C)nn2)cc1)CC3] |
| | | (SEQ ID NO: 2118) |
| LP15 | DXd | 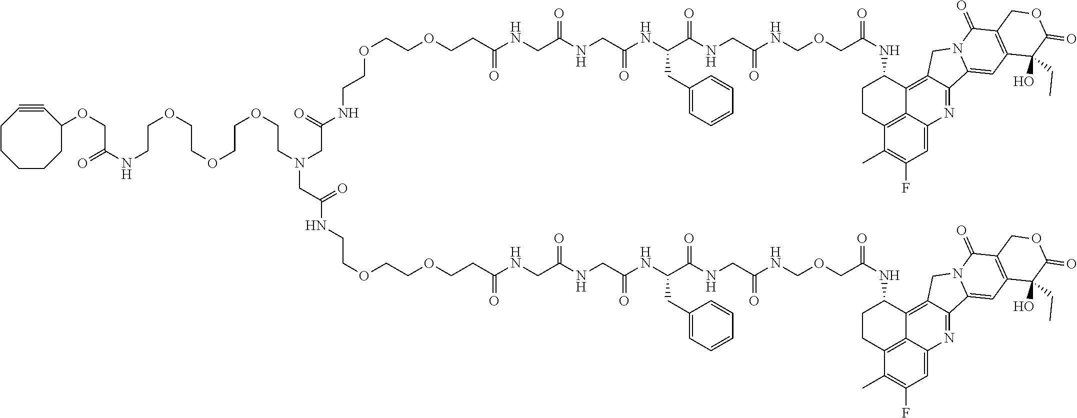 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCOCCOCCNC(=O)CN(CCOCCOCCOCCNC(=O)COC1C#CCCCCC1)CC(=O)NCCOCCOCCC(=O)NCC(=O)NCC(=O)N[C@@H](Cc1ccccc1)C(=O)NCC(=O)NCOCC(=O)N[C@H]1CCc2c(C)c(F)cc4nc5c(c1c24)Cn1c-5cc2c(c1=O)COC(=O)[C@]2(O)CC)CC3] |
| | | (SEQ ID NOS 2119 and 2119, respectively) |
| LP15C | P1 | 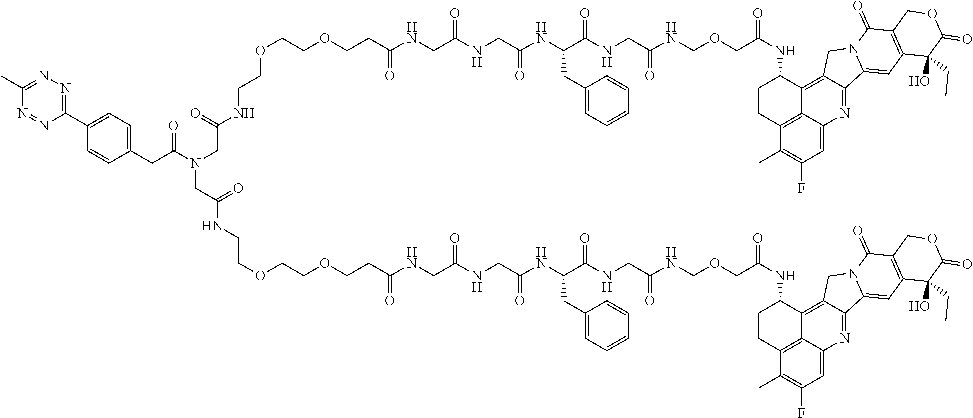 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCOCCOCCNC(=O)CN(CC(=O)NCCOCCOCCC(=O)NCC(=O)NCC(=O)N[C@@H](Cc1ccccc1)C(=O)NCC(=O)NCOCC(=O)N[C@H]1CCc2c(C)c(F)cc4nc5c(c1c24)Cn1c-5cc2c(c1=O)COC(=O)[C@]2(O)CC)C(=O)Cc1ccc(-c2nnc(C)nn2)cc1)CC3] |
| | | (SEQ ID NOS 2120 and 2120, respectively) |
| LP16 | P1 | 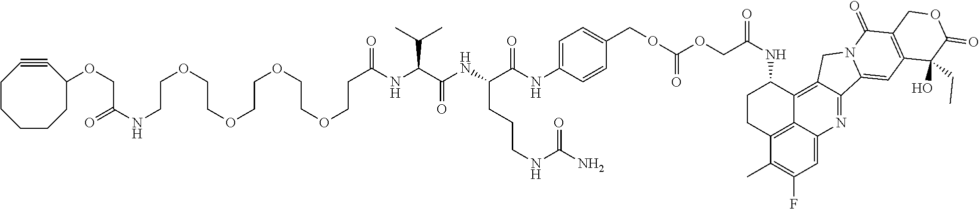 [SMILES: CC[C@@]1(O)C(=O)OCc2c1cc1n(c2=O)Cc2c-1nc1cc(F)c(C)c3c1c2[C@@H](NC(=O)COC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3] |
| LP17 | DXd | 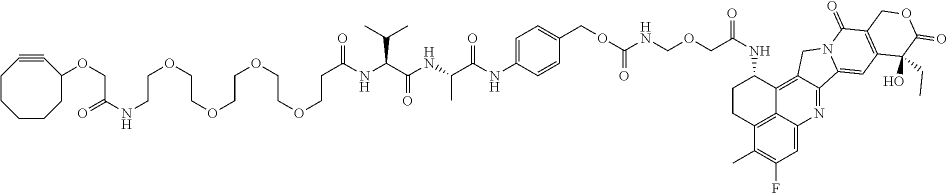 [SMILES: CC[C@@]1(O)C(=O)OCc2c1cc1n(c2=O)Cc2c-1nc1cc(F)c(C)c3c1c2[C@@H](NC(=O)COCNC(=O)OCc1ccc(NC(=O)[C@H](C)NC(=O)[C@@H](NC(=O)CCOCCOCCOCCOCCNC(=O)COC2C#CCCCCC2)C(C)C)cc1)CC3] |

Table 4, below, provides further characterization of non-limiting examples of the linker-payloads according to the present disclosure.

TABLE 4
List of Linker-ProDXds with corresponding Payloads

| | | | | MS | | | | |
|---|---|---|---|---|---|---|---|---|
| | | | | (M/2 + H)⁺ | HPLC | | | |
| | | | | (M/Z, | purity | | From | From |
| Cpd# | Description | MF | MW | 100%) | (%) | cLogP | ProDXd | ProDXd |
| LP1 | COT-PEG₄-vcPAB-Gly- | C₆₉H₉₀FN₁₁O₁₉ | 1396.5 | 699 | 99 | 0.72 | P1 | CP1190 |
| | NHCH₂-DXd | | | | | | | |
| LP1A | TCO-PEG₄-vcPAB-Gly- | C₆₈H₉₀FN₁₁O₁₉ | 1384.5 | 693 | 99 | 1.41 | P1 | CP1190 |
| | NHCH₂-DXd | | | | | | | |
| LP1B | MeTz-PEG₄-vcPAB-Gly- | C₆₈H₈₃FN₁₄O₁₈ | 1403.5 | 702 | 99 | 0.03 | P1 | CP1190 |
| | NHCH₂-DXd | | | | | | | |
| LP1D | COT-PEG₄-EvcPAB-Gly- | C₇₄H₉₇FN₁₂O₂₂ | 1525.65 | 764 | 99 | −0.28 | P1 | CP1190 |
| | NHCH₂-DXd | | | | | | | |
| LP1E | COT-PEG₄-Cysteic- | C₇₂H₉₅FN₁₂O₂₃S | 1547.67 | 774 | 97 | −3.87 | P1 | CP1190 |
| | vcPAB-Gly-NHCH₂-DXd | | | | | | | |
| LP2 | COT-PEG₄-vcPAB-Ser- | C₇₀H₉₂FN₁₁O₂₀ | 1426.5 | 714 | 98 | 0.24 | P2 | CP1412 |
| | NHCH₂-DXd | | | | | | | |
| LP3 | COT-PEG₄-vcPAB-Gln- | C₇₂H₉₅FN₁₂O₂₀ | 1467.6 | 734 | 98 | 0.13 | P3 | CP1408 |
| | NHCH₂-DXd | | | | | | | |
| LP4 | COT-PEG₄-vcPAB-Glu- | C₇₂H₉₄FN₁₁O₂₁ | 1468.6 | 735 | 97 | 0.81 | P4 | CP1409 |
| | NHCH₂-DXd | | | | | | | |
| LP5 | COT-PEG₄-vcPAB-Lys′- | C₇₃H₉₉FN₁₂O₁₉ | 1467.6 | 734 | >99 | 1.16 | P5 | CP1410 |
| | NHCH₂-DXd | | | | | | | |
| LP5C | MeTzCO-PEG₄-vcPAB- | C₇₄H₉₅FN₁₆O₁₈ | 1515.7 | 759 | 96 | −0.33 | P5 | CP1410 |
| | Lys′-NHCH₂-DXd | | | | | | | |
| LP5D | COT-PEG₄-EvcPAB-Lys′- | C₇₈H₁₀₆FN₁₃O₂₂ | 1596.8 | 799 | 97 | −2 | P5 | CP1410 |
| | NHCH₂-DXd | | | | | | | |
| LP7 | COT-PEG₄-vcPAB-Phe- | C₇₆H₉₆FN₁₁O₁₉ | 1486.6 | 744 | >99 | 2.95 | P7 | CP1411 |
| | NHCH₂-DXd | | | | | | | |
| LP9 | COT-PEG₄-vcPAB-Gly- | C₇₀H₉₂FN₁₁O₁₉ | 1410.6 | 706 | 95 | 0.94 | P9 | CP1422 |
| | NMeCH₂-DXd | | | | | | | |
| LP8 | COT-PEG₄-vcPAB-GlyGly- | C₇₁H₉₃FN₁₂O₂₀ | 1453.6 | 727 | 98 | −0.38 | P8 | CP1396 |
| | NHCH₂-DXd | | | | | | | |
| LP10 | COT-PEG₄-vcPAB-AIB- | C₇₁H₉₄FN₁₁O₁₉ | 1424.6 | 713 | 95 | 1.72 | P11 | CP1429 |
| | NHCH₂-DXd | | | | | | | |
| LP11 | COT-PEG₄-vcPAB-^{D}Phe- | C₇₆H₉₆FN₁₁O₁₉ | 1486.64 | 744 | 98 | 2.95 | P12 | CP1436 |
| | NHCH₂-DXd | | | | | | | |
| LP12D | COT-PEG₄-EvcPAB-Lys- | C₇₈H₁₀₆FN₁₃O₂₂ | 1596.77 | 799 | 95 | 1.99 | P5 | CP1410 |
| | NHCH₂-DXd | | | | | | | |
| LP13 | COT-PEG₂-BR1-(PEG₂- | C₁₄₆H₁₉₂F₂N₂₆O₄₅ | 3069.3 | 1024 | >95 | 4.55 | P1 | CP1190 |
| | EvcPAB-ProDXd)₂ | | | (M/3 + H) | | | | |
| LP13C | MeTz-Bn-BR1-(PEG₂- | C₁₁₃H₁₂₇F₂N₂₃O₂₉ | 2309.4 | 770 (M/3 + H) | >95 | 4.96 | P1 | CP1190 |
| | EvcPAB-ProDXd)₂ | | | | | | | |
| LP14 | COT-GGFG-NHCH₂-DXd | C₅₂H₅₇FN₈O₁₂ | 1005.07 | 1005 (M + H) | 97 | 0.38 | DXd | CP1178 |
| | (SEQ ID NO: 2117) | | | | | | | |
| LP14C | MeTz-Bn-GGFG NHCH₂- | C₅₃H₅₃FN₁₂O₁₁ | 1053.1 | 1053 (M + H) | >95 | −0.81 | DXd | CP1178 |
| | DXd (SEQ ID NO: 2118) | | | | | | | |
| LP15 | COT-PEG₃-BR2-(PEG₂- | C₁₂₀H₁₄₈F₂N₂₀O₃₃ | 2436.6 | 813 (M/3 + H) | >95 | −4.94 | DXd | CP1178 |
| | GGFG-NHCH₂-DXd)₂ | | | | | | | |
| | (SEQ ID NOS 2119 and | | | | | | | |
| | 2119, respectively) | | | | | | | |
| LP15C | MeTz-Bn-BR₂-(PEG₂- | C₆₈H₈₃FN₁₄O₁₈ | 1403.5 | 702 (M/2 + H) | >95 | −5.88 | DXd | CP1178 |
| | GGFG-NHCH₂-DXd)₂ | | | | | | | |
| | (SEQ ID NOS 2120 and | | | | | | | |
| | 2120, respectively) | | | | | | | |
| LP16 | COT-PEG₄-vcPABC-DXd | C₆₆H₈₄FN₉O₁₈ | 1310.4 | 1311 (M + H) | >95 | 2.49 | DXd | CP1178 |
| LP17 | COT-PEG₄-EvcPAB- | C₇₂H₉₄FN₁₁O₂₁ | 1468.6 | 735 | >95 | 0.83 | DXd | CP1178 |
| | NHCH₂-DXd | | | | | | | |

In one aspect, the present disclosure provides a compound of Formula (1-1):

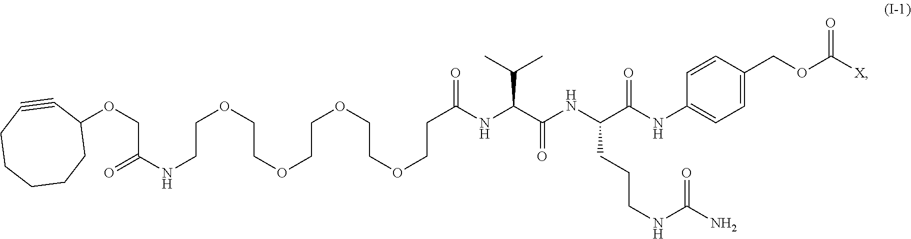

or a pharmaceutically acceptable salt thereof,
where
X is selected from the group consisting of

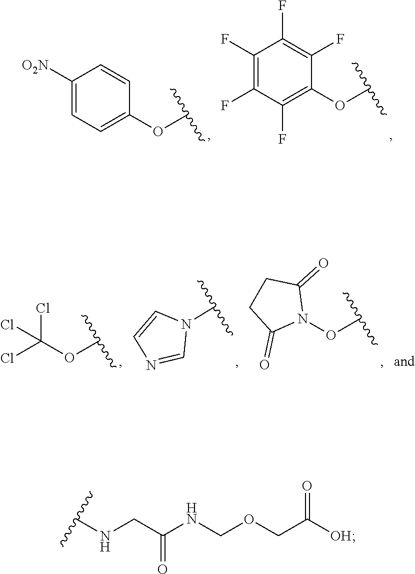

R¹, R², R³, and R⁴ are independently hydrogen or a C₁₋₅ alkyl, and
R⁵ and R⁶ are independently hydrogen, —NH₂, or a side chain of any natural or non-natural amino acid.

In one embodiment, the compound of Formula (1-1) has the following structure:

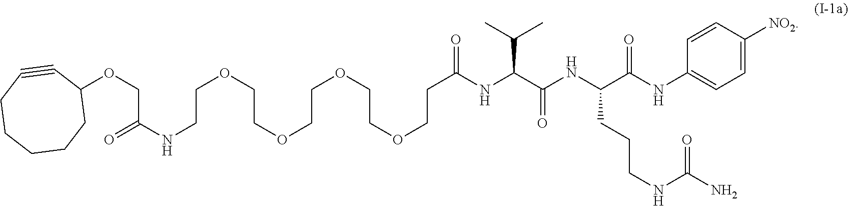 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc([N+](=O)[O-])cc1]

In one aspect, the present disclosure provides a compound of Formula (XVIII):

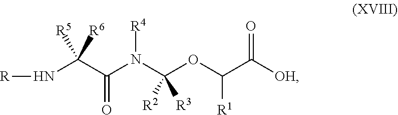

or a pharmaceutically acceptable salt thereof,
wherein R¹, R², R³, and R⁴ are independently hydrogen or a C₁₋₅ alkyl, and
R⁵ and R⁶ are independently hydrogen, —NH₂, or a side chain of any natural or non-natural amino acid.

In one embodiment, the compound of Formula (XVIII) has the following structure:

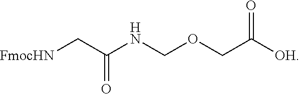

### Therapeutic Formulation and Administration

The present disclosure provides pharmaceutical compositions comprising the protein-drug conjugates of the present disclosure.

In one aspect, the present disclosure provides compositions comprising a population of protein-drug conjugates according to the present disclosure having a drug-antibody ratio (DAR) of about 0.5 to about 14.0.

In one embodiment, the composition has a DAR of about 1.0 to about 2.5.

In one embodiment, the composition has a DAR of about 2.

In one embodiment, the composition has a DAR of about 3.0 to about 4.5.

In one embodiment, the composition has a DAR of about 4.

In one embodiment, the composition has a DAR of about 6.5 to about 8.5.

In one embodiment, the composition has a DAR of about 8.

In one embodiment, the composition has a DAR of about 10 to about 14.

In one embodiment, the composition has a DAR of about 12.

The compositions of the disclosure are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of a protein-drug conjugate administered to a patient may vary depending upon the age and the size of the patient, target disease, conditions, route of administration, and the like. The suitable dose is typically calculated according to body weight or body surface area. When a protein-drug conjugate of the present disclosure is used for therapeutic purposes in an adult patient, it may be advantageous to intravenously administer the protein-drug conjugate of the present disclosure normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering a protein-drug conjugate may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™ OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present disclosure include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

### Therapeutic Uses of the Protein-Drug Conjugates, Linker-Payloads and Payloads

In another aspect, the protein-drug conjugates, e.g., ADCs, disclosed herein are useful, inter alia, for the treatment, prevention and/or amelioration of a disease, disorder or condition in need of such treatment.

In one embodiment, the present invention provides a method of treating a condition in a subject in need thereof comprising administering to the subject a therapeutically effective amount of a compound (e.g., an antibody-drug conjugate, a linker-payload and/or a payload) according to the disclosure, or the composition comprising any compound according to the present disclosure.

In one embodiment, the protein-drug conjugates, e.g., ADCs, disclosed herein are useful for treating cancer. In one embodiment, the protein-drug conjugates, e.g., ADCs, disclosed herein are useful for treating a cancer selected from the group consisting of breast cancer, ovarian cancer, prostate cancer, lung cancer, liver cancer, or brain cancer. In one embodiment, the protein-drug conjugates, e.g., ADCs, disclosed herein are useful for treating HER2+ breast cancer. In one embodiment, the protein-drug conjugates, e.g., ADCs, disclosed herein are useful for treating prostate cancer.

In one aspect, the present disclosure provides a method of selectively delivering a compound into a cell. In one embodiment, the method of selectively delivering a compound into a cell comprises linking the compound to a targeted antibody. In one embodiment, the compound is a payload as described above. In one embodiment, the cell is a mammalian cell. In one embodiment, the cell is a human cell. In one embodiment, the cell is a cancer cell. In one embodiment, the cancer cell is selected from the group consisting of a breast cancer cell, an ovarian cancer cell, a prostate cancer cell, a lung cancer cell, a liver cancer cell, or a brain cancer cell.

In certain embodiments, the present disclosure provides a method of selectively delivering into a cell a compound having the structure P-I:

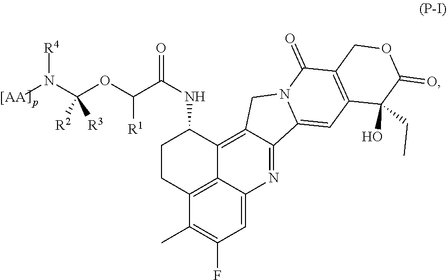

wherein R¹, R², R³, and R⁴ are independently hydrogen or a C₁₋₅ alkyl;
AA is a natural or a nonnatural amino acid; and
p is an integer from 1 to 6, or a pharmaceutically acceptable salt thereof.

In one aspect, the present disclosure provides a method of selectively targeting an antigen on a surface of a cell with a compound. In one embodiment, the method of selectively targeting an antigen on a surface of a cell with a compound comprises linking the compound to a targeted antibody. In one embodiment, the compound is a payload as described above. In one embodiment, the cell is a mammalian cell. In one embodiment, the cell is a human cell. In one embodiment, the cell is a cancer cell. In one embodiment, the cancer cell is selected from the group consisting of a breast cancer cell, an ovarian cancer cell, a prostate cancer cell, a lung cancer cell, a liver cancer cell, or a brain cancer cell.

In certain embodiments, the present disclosure provides a method of selectively targeting an antigen on a surface of a cell with a compound having the structure P-I:

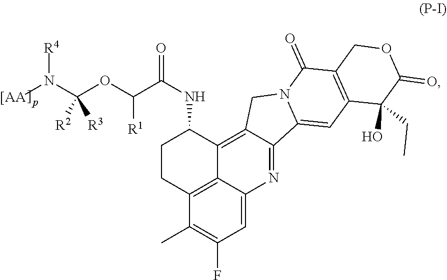

wherein R¹, R², R³, and R⁴ are independently hydrogen or a C₁₋₅ alkyl;
AA is a natural or a nonnatural amino acid; and
p is an integer from 1 to 6, or a pharmaceutically acceptable salt thereof.

In certain embodiments of any of the above methods, the compound having the structure P-I is selected from the group consisting of:

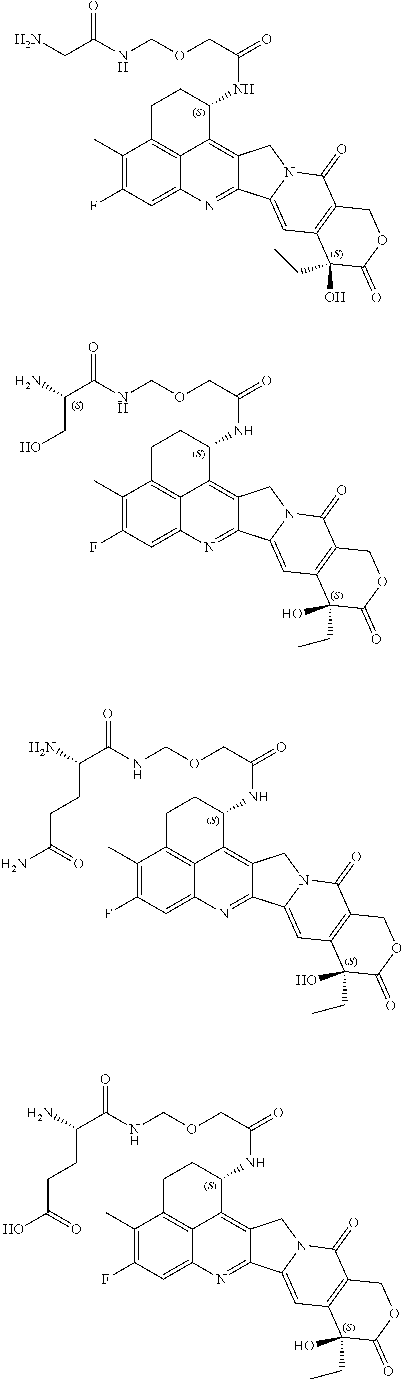

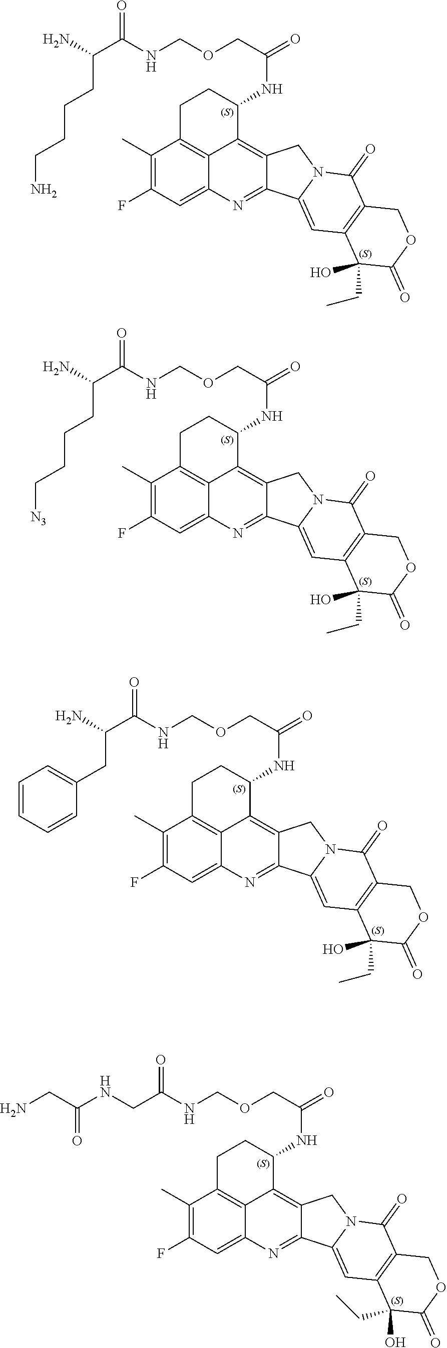

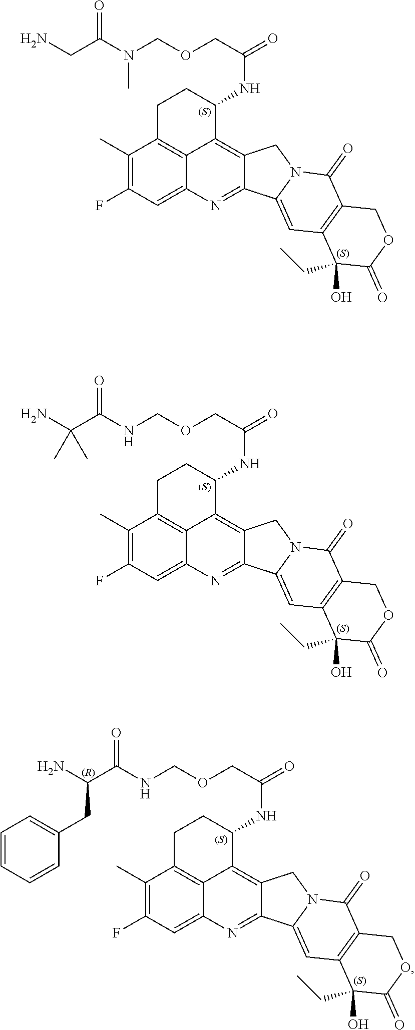

or a pharmaceutically acceptable salt thereof.

### Binding Agents

In one embodiment, the effectiveness of the protein-drug conjugate embodiments described herein depend on the selectivity of the binding agent to bind its binding partner. In one embodiment of the present disclosure, the binding agent is any molecule capable of binding with some specificity to a given binding partner. In one embodiment, the binding agent is within a mammal where the interaction can result in a therapeutic use. In an alternative embodiment, the binding agent is in vitro where the interaction can result in a diagnostic use. In some aspects, the binding agent is capable of binding to a cell or cell population.

Suitable binding agents of the present disclosure include proteins that bind to a binding partner, wherein the binding agent comprises one or more glutamine residues. Suitable binding agents include, but are not limited to, antibodies, lymphokines, hormones, growth factors, viral receptors, interleukins, or any other cell binding or peptide binding molecules or substances.

In one embodiment the binding agent is an antibody. In certain embodiments, the antibody is selected from monoclonal antibodies, polyclonal antibodies, antibody fragments (Fab, Fab′, and F(ab)2, minibodies, diabodies, triabodies, and the like). Antibodies herein can be humanized using methods described in U.S. Pat. No. 6,596,541 and US Publication No. 2012/0096572, each incorporated by reference in their entirety. In certain embodiments of the protein-drug conjugate compounds of the present disclosure, BA is a humanized monoclonal antibody. For example, BA can be a monoclonal antibody that binds HER2, MET, or STEAP2. In certain embodiments of the protein-drug conjugate compounds of the present disclosure, BA is a bispecific antibody, e.g., an anti-HER2/HER2 bispecific antibody, or an anti-MET/MET bispecific antibody.

In the present disclosure, the antibody can be any antibody deemed suitable to the practitioner of skill. In some embodiments, the antibody comprises at least one glutamine residue in at least one polypeptide chain sequence. In certain embodiments, the antibody comprises one or more gln295 residues. In certain embodiments, the antibody comprises two heavy chain polypeptides, each with one gln295 residue. In further embodiments, the antibody comprises one or more glutamine residues at a site other than a heavy chain 295. Such antibodies can be isolated from natural sources or engineered to comprise one or more glutamine residues. Techniques for engineering glutamine residues into an antibody polypeptide chain are within the skill of the practitioners in the art. In certain embodiments, the antibody is aglycosylated.

The antibody can be in any form known to those of skill in the art. In certain embodiments, the antibody comprises a light chain. In certain embodiments, the light chain is a kappa light chain. In certain embodiments, the light chain is a lambda light chain.

In certain embodiments, the antibody comprises a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the antibody is an antibody fragment. In some aspects, the antibody fragment is an Fv fragment. In some aspects, the antibody fragment is a Fab fragment. In some aspects, the antibody fragment is a F(ab′)2 fragment. In some aspects, the antibody fragment is a Fab′ fragment. In some aspects, the antibody fragment is an scFv (sFv) fragment. In some aspects, the antibody fragment is an scFv-Fc fragment.

In some embodiments, the antibody is a monoclonal antibody. In some embodiments, the antibody is a polyclonal antibody.

In some embodiments, the antibody is a chimeric antibody. In some embodiments, the antibody is a humanized antibody. In some embodiments, the antibody is a human antibody.

The antibody can have binding specificity for any antigen deemed suitable to those of skill in the art. In certain embodiments, the antigen is a transmembrane molecule (e.g., receptor) or a growth factor. Exemplary antigens include, but are not limited to, molecules such as renin; a growth hormone, including human growth hormone and bovine growth hormone; growth hormone releasing factor; parathyroid hormone; thyroid stimulating hormone; lipoproteins; alpha1-antitrypsin; insulin A-chain; insulin B-chain; proinsulin; follicle stimulating hormone; calcitonin; luteinizing hormone; glucagon; clotting factors such as factor vmc, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin, such as human serum albumin; Muellerian-inhibiting substance; relaxin A-chain; relaxin B-chain; prorelaxin; mouse gonadotropin-associated peptide; a microbial protein, such as betalactamase; DNase; 19E; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT4, NT-5, or NT-6), or a nerve growth factor such as NGF-β; platelet-derived growth factor (PDGF); fibroblast growth factor such as aFGF and bFGF; fibroblast growth factor receptor 2 (FGFR2), epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -2 (IGF-I and IGF-2); des(I-3)-IGF-I (brain IGF-I), insulin-like growth factor binding proteins, EpCAM, gD3, FLT3, PSMA, PSCA, MUC1, MUC16, STEAP, STEAP2, CEA, TENB2, EphA receptors, EphB receptors, folate receptor, FOLRI, mesothelin, cripto, alphavbeta6, integrins, VEGF, VEGFR, EGFR, transferrin receptor, IRTAI, IRTA2, IRTA3, IRTA4, IRTA5; CD proteins such as CD2, CD3, CD4, CD5, CD6, CD8, CDII, CD14, CD19, CD20, CD21, CD22, CD25, CD26, CD28, CD30, CD33, CD36, CD37, CD38, CD40, CD44, CD52, CD55, CD56, CD59, CD70, CD79, CD80, CD81, CD103, CD105, CD134, CD137, CD138, CD152, or an antibody which binds to one or more tumor-associated antigens or cell-surface receptors disclosed in US Publication No. 2008/0171040 or US Publication No. 2008/0305044 and incorporated in their entirety by reference; erythropoietin; osteoinductive factors; immunotoxins; a bone morphogenetic protein (BMP); an interferon, such as interferon-alpha, -beta, and -gamma; colony stimulating factors (CSFs), e.g., M-CSF, gM-CSF, and g-CSF; interleukins (ILs), e.g., IL-1 to IL-10; superoxide dismutase; T-cell receptors; surface membrane proteins; decay accelerating factor; viral antigen such as, for example, a portion of the HIV envelope; transport proteins; homing receptors; addressins; regulatory proteins; integrins, such as CDIIa, CDIIb, CDIIc, CD18, an ICAM, VLA-4 and VCAM; a tumor associated antigen such as AFP, ALK, B7H4, BAGE proteins, β-catenin, brc-abl, BRCA1, BORIS, CA9 (carbonic anhydrase IX), caspase-8, CD20, CD40, CD123, CDK4, CEA, CLEC12A, c-kit, cMET, CTLA4, cyclin-B1, CYP1B1, EGFR, EGFRVIII, endoglin, Epcam, EphA2, ErbB2/HER2, ErbB3/HER3, ErbB4/HER4, ETV6-AML, Fra-1, FOLR1, gAGE proteins (e.g., gAGE-1, -2), gD2, gD3, globoH, glypican-3, gM3, gp100, HER2, HLA/B-raf, HLA/EBNA1, HLA/k-ras, HLA/MAGE-A3, hTERT, IGF1R, LGR5, LMP2, MAGE proteins (e.g., MAGE-1, -2, -3, -4, -6, and -12), MART-1, mesothelin, mL-IAP, Muc1, Muc16 (CA-125), MET, MUM1, NA17, NGEP, NY-BR1, NY-BR62, NY-BR85, NY-ESO1, OX40, p15, p53, PAP, PAX3, PAX5, PCTA-1, PDGFR-α, PDGFR-β, PDGF-A, PDGF-B, PDGF-C, PDGF-D, PLAC1, PRLR, PRAME, PSCA, PSGR, PSMA (FOLH1), RAGE proteins, Ras, RGS5, Rho, SART-1, SART-3, STEAP1, STEAP2, STn, survivin, TAG-72, TGF-β, TMPRSS2, Tn, TNFRSF17, TRP-1, TRP-2, tyrosinase, and uroplakin-3, and fragments of any of the herein-listed polypeptides.

Exemplary antigens also include, but are not limited to, BCMA, SLAMF7, B7H4, gPNMB, UPK3A, and LGR5. Exemplary antigens also include, but are not limited to, MUC16, PSMA, STEAP2, and HER2.

In some embodiments, antigens also include, but are not limited to, hematologic targets, e.g., CD22, CD30, CD33, CD79a, and CD79b.

Some embodiments herein are target specific for therapeutic or diagnostic use. In one embodiment, binding agents are prepared to interact with and bind to antigens defined as tumor antigens, which include antigens specific for a type of tumor or antigens that are shared, overexpressed or modified on a particular type of tumor. Examples include: alpha-actinin-4 with lung cancer, ARTC1 with melanoma, BCR-ABL fusion protein with chronic myeloid leukemia, B-RAF, CLPP or Cdc27 with melanoma, CASP-8 with squamous cell carcinoma, and hsp70-2 with renal cell carcinoma as well as the following shared tumor-specific antigens, for example: BAGE-1, gAGE, gnTV, KK-LC-1, MAGE-A2, NA88-A, TRP2-INT2. In some embodiments, the antigen is PRLR or HER2. In some embodiments, the antibody binds STEAP2, MUC16, EGFR, EGFRVIII, FGR2, or PRLR.

In some embodiments, the antigens include HER2. In some embodiments, the antigens include STEAP2. In some embodiments, the antigens include MET. In some embodiments, the antigens include EGFRVIII. In some embodiments, the antigens include MUC16. In some embodiments, the antigens include PRLR. In some embodiments, the antigens include PSMA. In some embodiments, the antigens include FGFR2.

In some embodiments, the BA is an anti-HER2 antibody, an anti-STEAP2 antibody, an anti-MET antibody, an anti-EGFRVIII antibody, an anti-MUC16 antibody, an anti-PRLR antibody, an anti-PSMA antibody, or an anti-FGFR2 antibody, an anti-HER2/HER2 bispecific antibody, an anti-MET/MET bispecific antibody, or an anti-FOLR1 antibody, or an antigen-binding fragment thereof.

In some embodiments, the BA targets a cancer selected from the group consisting of breast cancer, ovarian cancer, prostate cancer, lung cancer, liver cancer, or brain cancer. Anti-HER2 Antibodies Suitable for Protein-Drug Conjugates

In some embodiments, the antibody is an anti HER2 antibody. In some embodiments, the antibody is trastuzumab, pertuzumab (2C4) or margetuximab (MGAH22). In some embodiment, the antibody is trastuzumab. According to certain embodiments, protein-drug conjugates, e.g., ADCs, according to the disclosure comprise anti-HER2 antibody. In some embodiment, the anti-HER2 antibody may include those described in WO 2019/212965 A1.

In some embodiments, the antibody is an anti-HER2/HER2 bispecific antibody, which comprises a first antigen-binding domain (D1) which specifically binds a first epitope of human HER2 and a second antigen-binding domain (D2) which specifically binds a second epitope of human HER2.

In certain embodiments, D1 and D2 domains of an anti-HER2/HER2 bispecific antibody are non-competitive with one another. Non-competition between D1 and D2 for binding to HER2 means that, the respective monospecific antigen binding proteins from which D1 and D2 were derived do not compete with one another for binding to human HER2. Exemplary antigen-binding protein competition assays are known in the art.

In certain embodiments, D1 and D2 bind to different (e.g., non-overlapping, or partially overlapping) epitopes on HER2.

In one non-limiting embodiment, the present disclosure provides protein-drug conjugates comprising a bispecific antigen-binding molecule comprising:
a first antigen-binding domain (D1); and
a second antigen-binding domain (D2);
wherein D1 specifically binds a first epitope of human HER2; and
wherein D2 specifically binds a second epitope of human HER2.

Anti-HER2/HER2 bispecific antibodies may be constructed using the antigen-binding domains of two separate monospecific anti-HER2 antibodies. For example, a collection of monoclonal monospecific anti-HER2 antibodies may be produced using standard methods known in the art. The individual antibodies thus produced may be tested pairwise against one another for cross-competition to a HER2 protein. If two different anti-HER2 antibodies are able to bind to HER2 at the same time (i.e., do not compete with one another), then the antigen-binding domain from the first anti-HER2 antibody and the antigen-binding domain from the second, non-competitive anti-HER2 antibody can be engineered into a single anti-HER2/HER2 bispecific antibody in accordance with the present disclosure.

According to the present disclosure, a bispecific antigen-binding molecule can be a single multifunctional polypeptide, or it can be a multimeric complex of two or more polypeptides that are covalently or non-covalently associated with one another. As will be made evident by the present disclosure, any antigen binding construct which has the ability to simultaneously bind two separate, non-identical epitopes of the HER2 molecule is regarded as a bispecific antigen-binding molecule. Any of the bispecific antigen-binding molecules described herein, or variants thereof, may be constructed using standard molecular biological techniques (e.g., recombinant DNA and protein expression technology) as will be known to a person of ordinary skill in the art.

In another aspect, the disclosure provides a pharmaceutical composition comprising a recombinant human antibody or fragment thereof which specifically binds HER2 and a pharmaceutically acceptable carrier. In one non-limiting embodiment, the antibody may bind two separate epitopes on the HER2 protein, i.e., the antibody is a HER2/HER2 bispecific antibody. In a related aspect, the disclosure features a composition which is a combination of an anti-HER2/HER2 antibody and a second therapeutic agent. In one embodiment, the second therapeutic agent is any agent that is advantageously combined with an anti-HER2/HER2 antibody. Additional combination therapies and co-formulations involving the anti-HER2/HER2 bispecific antibodies of the present disclosure are disclosed elsewhere herein.

In another aspect, the disclosure provides therapeutic methods for targeting/killing tumor cells expressing HER2 using an anti-HER2/HER2 bispecific antibody of the disclosure, wherein the therapeutic methods comprise administering a therapeutically effective amount of a pharmaceutical composition comprising an anti-HER2/HER2 antibody of the disclosure to a subject in need thereof. In some cases, the anti-HER2/HER2 antibodies (or antigen-binding fragments thereof) can be used for treating breast cancer, or may be modified to be more cytotoxic by methods, including but not limited to, modified Fc domains to increase ADCC (see e.g., Shield et al. (2002) JBC 277:26733), radioimmunotherapy, antibody-drug conjugates, or other methods for increasing the efficiency of tumor ablation.

The present disclosure also includes the use of an anti-HER2 antibody of the disclosure in the manufacture of a medicament for the treatment of a disease or disorder (e.g., cancer) related to or caused by HER2-expressing cells. In one aspect, the disclosure relates to a compound comprising an anti-HER2 antibody or antigen-binding fragment, or a HER2/HER2 bispecific antibody, as disclosed herein, for use in medicine. In one aspect, the disclosure relates to a compound comprising an antibody-drug conjugate (ADC) as disclosed herein, for use in medicine.

In yet another aspect, the disclosure provides bispecific anti-HER2/HER2 antibodies for diagnostic applications, such as, e.g., imaging reagents.

### Anti-STEAP2 Antibodies Suitable for Protein-Drug Conjugates

In some embodiments, the antibody is an anti-six-transmembrane epithelial antigen of prostate 2 (STEAP2), i.e., an anti-STEAP2 antibody. STEAP2, which works as a shuttle between the Golgi complex and the plasma membrane, is a metalloreductase which reduces iron and copper, facilitating their import into the cell. STEAP2 is mainly localized to epithelial cells of the prostate. STEAP2 is also expressed in normal heart, brain, pancreas, ovary, skeletal muscle, mammary gland, testis, uterus, kidney, lung, trachea, colon, and liver. STEAP2 is over-expressed in cancerous tissues, including prostate, bladder, cervix, lung, colon, kidney, breast, pancreatic, stomach, uterus, and ovarian tumors (Gomes, I. M. et al., 2012, Mol. Cancer Res. 10:573-587; Challita-Eid-P.M., et al., 2003, WO 03/087306; Emtage, P. C. R., 2005, WO 2005/079490).

In one aspect, suitable anti-STEAP antibodies are those disclosed in US2018/0104357. Exemplary anti-STEAP2 antibodies according to the present disclosure are listed in Tables 5 and 6 herein. Table 5 sets forth the amino acid sequence identifiers of the heavy chain variable regions (HCVRs) and light chain variable regions (LCVRs), as well as heavy chain complementarity determining regions (HCDR1, HCDR2 and HCDR3), and light chain complementarity determining regions (LCDR1, LCDR2 and LCDR3) of the exemplary anti-STEAP2 antibodies. Table 6 sets forth the sequence identifiers of the nucleic acid molecules encoding the HCVRs, LCVRs, HCDR1, HCDR2 HCDR3, LCDR1, LCDR2 and LCDR3 of the exemplary anti-STEAP2 antibodies.

The present disclosure provides antibodies, or antigen-binding fragments thereof, comprising an HCVR comprising an amino acid sequence selected from any of the HCVR amino acid sequences listed in Table 5, or a substantially similar sequence thereof having at least 90%, at least 95%, at least 98% or at least 99% sequence identity thereto.

The present disclosure also provides antibodies, or antigen-binding fragments thereof, comprising an LCVR comprising an amino acid sequence selected from any of the LCVR amino acid sequences listed in Table 5, or a substantially similar sequence thereof having at least 90%, at least 95%, at least 98% or at least 99% sequence identity thereto.

The present disclosure also provides antibodies, or antigen-binding fragments thereof, comprising an HCVR and an LCVR amino acid sequence pair (HCVR/LCVR) comprising any of the HCVR amino acid sequences listed in Table 5 paired with any of the LCVR amino acid sequences listed in Table 5. According to certain embodiments, the present disclosure provides antibodies, or antigen-binding fragments thereof, comprising an HCVR/LCVR amino acid sequence pair contained within any of the exemplary anti-STEAP2 antibodies listed in Table 5. In certain embodiments, the HCVR/LCVR amino acid sequence pair is selected from the group consisting of SEQ ID NOs: 250/258 (e.g., H2M11162N).

The present disclosure also provides antibodies, or antigen-binding fragments thereof, comprising a heavy chain CDR1 (HCDR1) comprising an amino acid sequence selected from any of the HCDR1 amino acid sequences listed in Table 5 or a substantially similar sequence thereof having at least 90%, at least 95%, at least 98% or at least 99% sequence identity.

The present disclosure also provides antibodies, or antigen-binding fragments thereof, comprising a heavy chain CDR2 (HCDR2) comprising an amino acid sequence selected from any of the HCDR2 amino acid sequences listed in Table 5 or a substantially similar sequence thereof having at least 90%, at least 95%, at least 98% or at least 99% sequence identity.

The present disclosure also provides antibodies, or antigen-binding fragments thereof, comprising a heavy chain CDR3 (HCDR3) comprising an amino acid sequence selected from any of the HCDR3 amino acid sequences listed in Table 5 or a substantially similar sequence thereof having at least 90%, at least 95%, at least 98% or at least 99% sequence identity.

The present disclosure also provides antibodies, or antigen-binding fragments thereof, comprising a light chain CDR1 (LCDR1) comprising an amino acid sequence selected from any of the LCDR1 amino acid sequences listed in Table 5 or a substantially similar sequence thereof having at least 90%, at least 95%, at least 98% or at least 99% sequence identity.

The present disclosure also provides antibodies, or antigen-binding fragments thereof, comprising a light chain CDR2 (LCDR2) comprising an amino acid sequence selected from any of the LCDR2 amino acid sequences listed in Table 5 or a substantially similar sequence thereof having at least 90%, at least 95%, at least 98% or at least 99% sequence identity.

The present disclosure also provides antibodies, or antigen-binding fragments thereof, comprising a light chain CDR3 (LCDR3) comprising an amino acid sequence selected from any of the LCDR3 amino acid sequences listed in Table 5 or a substantially similar sequence thereof having at least 90%, at least 95%, at least 98% or at least 99% sequence identity.

The present disclosure also provides antibodies, or antigen-binding fragments thereof, comprising an HCDR3 and an LCDR3 amino acid sequence pair (HCDR3/LCDR3) comprising any of the HCDR3 amino acid sequences listed in Table 5 paired with any of the LCDR3 amino acid sequences listed in Table 5. According to certain embodiments, the present disclosure provides antibodies, or antigen-binding fragments thereof, comprising an HCDR3/LCDR3 amino acid sequence pair contained within any of the exemplary anti-STEAP2 antibodies listed in Table 5. In certain embodiments, the HCDR3/LCDR3 amino acid sequence pair is selected from the group consisting of SEQ ID NOs: 256/264 (e.g., H2M11162N).

The present disclosure also provides antibodies, or antigen-binding fragments thereof, comprising a set of six CDRs (i.e., HCDR1-HCDR2-HCDR3-LCDR1-LCDR2-LCDR3) contained within any of the exemplary anti-STEAP2 antibodies listed in Table 5. In certain embodiments, the HCDR1-HCDR2-HCDR3-LCDR1-LCDR2-LCDR3 amino acid sequences set is selected from the group consisting of SEQ ID NOs: 252-254-256-260-262-264 (e.g., H2M 11162N).

In a related embodiment, the present disclosure provides antibodies, or antigen-binding fragments thereof, comprising a set of six CDRs (i.e., HCDR1-HCDR2-HCDR3-LCDR1-LCDR2-LCDR3) contained within an HCVR/LCVR amino acid sequence pair as defined by any of the exemplary anti-STEAP2 antibodies listed in Table 5. For example, the present disclosure includes antibodies, or antigen-binding fragments thereof, comprising the HCDR1-HCDR2-HCDR3-LCDR1-LCDR2-LCDR3 amino acid sequences set contained within an HCVR/LCVR amino acid sequence pair selected from the group consisting of SEQ ID NOs: 250/258 (e.g., H2M11162N). Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

The present disclosure also provides nucleic acid molecules encoding anti-STEAP2 antibodies or portions thereof. For example, the present disclosure provides nucleic acid molecules encoding any of the HCVR amino acid sequences listed in Table 5; in certain embodiments the nucleic acid molecule comprises a polynucleotide sequence selected from any of the HCVR nucleic acid sequences listed in Table 6, or a substantially similar sequence thereof having at least 90%, at least 95%, at least 98% or at least 99% sequence identity thereto.

The present disclosure also provides nucleic acid molecules encoding any of the LCVR amino acid sequences listed in Table 5; in certain embodiments the nucleic acid molecule comprises a polynucleotide sequence selected from any of the LCVR nucleic acid sequences listed in Table 6, or a substantially similar sequence thereof having at least 90%, at least 95%, at least 98% or at least 99% sequence identity thereto.

The present disclosure also provides nucleic acid molecules encoding any of the HCDR1 amino acid sequences listed in Table 5; in certain embodiments the nucleic acid molecule comprises a polynucleotide sequence selected from any of the HCDR1 nucleic acid sequences listed in Table 6, or a substantially similar sequence thereof having at least 90%, at least 95%, at least 98% or at least 99% sequence identity thereto.

The present disclosure also provides nucleic acid molecules encoding any of the HCDR2 amino acid sequences listed in Table 5; in certain embodiments the nucleic acid molecule comprises a polynucleotide sequence selected from any of the HCDR2 nucleic acid sequences listed in Table 6, or a substantially similar sequence thereof having at least 90%, at least 95%, at least 98% or at least 99% sequence identity thereto.

The present disclosure also provides nucleic acid molecules encoding any of the HCDR3 amino acid sequences listed in Table 5; in certain embodiments the nucleic acid molecule comprises a polynucleotide sequence selected from any of the HCDR3 nucleic acid sequences listed in Table 6, or a substantially similar sequence thereof having at least 90%, at least 95%, at least 98% or at least 99% sequence identity thereto.

The present disclosure also provides nucleic acid molecules encoding any of the LCDR1 amino acid sequences listed in Table 5; in certain embodiments the nucleic acid molecule comprises a polynucleotide sequence selected from any of the LCDR1 nucleic acid sequences listed in Table 6, or a substantially similar sequence thereof having at least 90%, at least 95%, at least 98% or at least 99% sequence identity thereto.

The present disclosure also provides nucleic acid molecules encoding any of the LCDR2 amino acid sequences listed in Table 5; in certain embodiments the nucleic acid molecule comprises a polynucleotide sequence selected from any of the LCDR2 nucleic acid sequences listed in Table 6, or a substantially similar sequence thereof having at least 90%, at least 95%, at least 98% or at least 99% sequence identity thereto.

The present disclosure also provides nucleic acid molecules encoding any of the LCDR3 amino acid sequences listed in Table 5; in certain embodiments the nucleic acid molecule comprises a polynucleotide sequence selected from any of the LCDR3 nucleic acid sequences listed in Table 6, or a substantially similar sequence thereof having at least 90%, at least 95%, at least 98% or at least 99% sequence identity thereto.

The present disclosure also provides nucleic acid molecules encoding an HCVR, wherein the HCVR comprises a set of three CDRs (i.e., HCDR1-HCDR2-HCDR3), wherein the HCDR1-HCDR2-HCDR3 amino acid sequence set is as defined by any of the exemplary anti-STEAP2 antibodies listed in Table 5.

The present disclosure also provides nucleic acid molecules encoding an LCVR, wherein the LCVR comprises a set of three CDRs (i.e., LCDR1-LCDR2-LCDR3), wherein the LCDR1-LCDR2-LCDR3 amino acid sequence set is as defined by any of the exemplary anti-STEAP2 antibodies listed in Table 5.

The present disclosure also provides nucleic acid molecules encoding both an HCVR and an LCVR, wherein the HCVR comprises an amino acid sequence of any of the HCVR amino acid sequences listed in Table 5, and wherein the LCVR comprises an amino acid sequence of any of the LCVR amino acid sequences listed in Table 5. In certain embodiments, the nucleic acid molecule comprises a polynucleotide sequence selected from any of the HCVR nucleic acid sequences listed in Table 6, or a substantially similar sequence thereof having at least 90%, at least 95%, at least 98% or at least 99% sequence identity thereto, and a polynucleotide sequence selected from any of the LCVR nucleic acid sequences listed in Table 6, or a substantially similar sequence thereof having at least 90%, at least 95%, at least 98% or at least 99% sequence identity thereto. In certain embodiments according to this aspect of the disclosure, the nucleic acid molecule encodes an HCVR and LCVR, wherein the HCVR and LCVR are both derived from the same anti-STEAP2 antibody listed in Table 5.

The present disclosure also provides recombinant expression vectors capable of expressing a polypeptide comprising a heavy or light chain variable region of an anti-STEAP2 antibody. For example, the present disclosure includes recombinant expression vectors comprising any of the nucleic acid molecules mentioned above, i.e., nucleic acid molecules encoding any of the HCVR, LCVR, and/or CDR sequences as set forth in Table 5. Also included within the scope of the present disclosure are host cells into which such vectors have been introduced, as well as methods of producing the antibodies or portions thereof by culturing the host cells under conditions permitting production of the antibodies or antibody fragments, and recovering the antibodies and antibody fragments so produced.

The present disclosure includes anti-STEAP2 antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

In another aspect, the disclosure provides a pharmaceutical composition comprising a recombinant human antibody or fragment thereof which specifically binds STEAP2 and a pharmaceutically acceptable carrier. In a related aspect, the disclosure features a composition which is a combination of an anti-STEAP2 antibody and a second therapeutic agent. In one embodiment, the second therapeutic agent is any agent that is advantageously combined with an anti-STEAP2 antibody. Additional combination therapies and co-formulations involving the anti-STEAP2 antibodies of the present disclosure are disclosed elsewhere herein.

In another aspect, the disclosure provides therapeutic methods for targeting/killing tumor cells expressing STEAP2 using an anti-STEAP2 antibody of the disclosure, wherein the therapeutic methods comprise administering a therapeutically effective amount of a pharmaceutical composition comprising an anti-STEAP2 antibody of the disclosure to a subject in need thereof. In some cases, the anti-STEAP2 antibodies (or antigen-binding fragments thereof) can be used for treating prostate cancer, or may be modified to be more cytotoxic by methods, including but not limited to, modified Fc domains to increase ADCC (see e.g., Shield et al. (2002) JBC 277:26733), radioimmunotherapy, antibody-drug conjugates, or other methods for increasing the efficiency of tumor ablation.

The present disclosure also includes the use of an anti-STEAP2 antibody of the disclosure in the manufacture of a medicament for the treatment of a disease or disorder (e.g., cancer) related to or caused by STEAP2-expressing cells. In one aspect, the disclosure relates to a compound comprising an anti-STEAP2 antibody or antigen-binding fragment, or a STEAP2×CD3 bispecific antibody, as disclosed herein, for use in medicine. In one aspect, the disclosure relates to a compound comprising an antibody-drug conjugate (ADC) as disclosed herein, for use in medicine.

In yet another aspect, the disclosure provides monospecific anti-STEAP2 antibodies for diagnostic applications, such as, e.g., imaging reagents.

In yet another aspect, the disclosure provides therapeutic methods for stimulating T cell activation using an anti-CD3 antibody or antigen-binding portion of an antibody of the disclosure, wherein the therapeutic methods comprise administering a therapeutically effective amount of a pharmaceutical composition comprising an antibody

In another aspect, the present disclosure provides an isolated antibody or antigen-binding fragment thereof that binds STEAP2-expressing C4-2 cells with an EC50 of less than 50 nM as measured by FACS analysis. In another aspect, the present disclosure provides an isolated antibody or antigen-binding fragment thereof that binds and is internalized by STEAP2-expressing C4-2 cells.

The disclosure further provides an antibody or antigen-binding fragment that competes for binding to human STEAP2 with a reference antibody comprising an HCVR/LCVR amino acid sequence pair as set forth in Table 5. In another aspect, the disclosure provides an antibody or antigen-binding fragment that competes for binding to human STEAP2 with a reference antibody comprising an HCVR/LCVR amino acid sequence pair selected from the group consisting of SEQ ID NOs:2/10; 18/26; 34/42; 50/58; 66/58; 74/58; 82/58; 90/58; 98/58; 106/114; 122/130; 138/146; 154/162; 170/178; 186/194; 202/210; 218/226; 234/242; 250/258; 266/274; 282/290; 298/306; 314/322; 330/338; 346/354; 362/370; and 378/386.

The disclosure furthermore provides an antibody or antigen-binding fragment, wherein the antibody or antigen-binding fragment thereof binds to the same epitope on human STEAP2 as a reference antibody comprising an HCVR/LCVR amino acid sequence pair as set forth in Table 5. In another aspect, the antibody or antigen-binding fragment binds to the same epitope on human STEAP2 as a reference antibody comprising an HCVR/LCVR amino acid sequence pair selected from the group consisting of SEQ ID NOs:2/10; 18/26; 34/42; 50/58; 66/58; 74/58; 82/58; 90/58; 98/58; 106/114; 122/130; 138/146; 154/162; 170/178; 186/194; 202/210; 218/226; 234/242; 250/258; 266/274; 282/290; 298/306; 314/322; 330/338; 346/354; 362/370; and 378/386.

The disclosure further provides an isolated antibody or antigen-binding fragment thereof that binds human STEAP2, wherein the antibody or antigen-binding fragment comprises: the complementarity determining regions (CDRs) of a heavy chain variable region (HCVR) having an amino acid sequence as set forth in Table 5; and the CDRs of a light chain variable region (LCVR) having an amino acid sequence as set forth in Table 5. In another aspect, the isolated antibody or antigen-binding fragment comprises the heavy and light chain CDRs of a HCVR/LCVR amino acid sequence pair selected from the group consisting of: SEQ ID NOs:2/10; 18/26; 34/42; 50/58; 66/58; 74/58; 82/58; 90/58; 98/58; 106/114; 122/130; 138/146; 154/162; 170/178; 186/194; 202/210; 218/226; 234/242; 250/258; 266/274; 282/290; 298/306; 314/322; 330/338; 346/354; 362/370; and 378/386. In yet another aspect, the isolated antibody or antigen-binding fragment comprises HCDR1-HCDR2-HCDR3-LCDR1-LCDR2-LCDR3 domains, respectively, selected from the group consisting of: SEQ ID NOs:4-6-8-12-14-16; 20-22-24-28-30-32; 36-38-40-44-46-48; 52-54-56-60-62-64; 68-70-72-60-62-64; 76-78-80-60-62-64; 84-86-88-60-62-64; 92-94-96-60-62-64; 100-102-104-60-62-64; 108-110-112-116-118-120; 124-126-128-132-134-136; 140-142-144-148-150-152; 156-158-160-164-166-168; 172-174-176-180-182-184; 188-190-192-196-198-200; 204-206-208-212-214-216; 220-222-224-228-230-232; 236-238-240-244-246-248; 252-254-256-260-262-264; 268-270-272-276-278-280; 284-286-288-292-294-296; 300-302-304-308-310-312; 316-318-320-324-326-328; 332-334-336-340-342-344; 348-350-352-356-358-360; 364-366-368-372-374-376; and 380-382-384-388-390-392.

In another aspect, the disclosure provides an isolated antibody or antigen-binding fragment thereof that binds human STEAP2, wherein the antibody or antigen-binding fragment comprises: (a) a heavy chain variable region (HCVR) having an amino acid sequence selected from the group consisting of SEQ ID NOs: 2, 18, 34, 50, 66, 74, 82, 90, 98, 106, 122, 138, 154, 170, 186, 202, 218, 234, 250, 266, 282, 298, 314, 330, 346, 362, and 378; and (b) a light chain variable region (LCVR) having an amino acid sequence selected from the group consisting of SEQ ID NOs: 10; 26; 42; 58 114; 130; 146; 162; 178; 194; 210; 226, 242; 258; 274; 290; 306; 322; 338; 354; 370; and 386. In a further aspect, the isolated antibody or antigen-binding fragment of claim 10, wherein the antibody or antigen-binding fragment comprises a HCVR/LCVR amino acid sequence pair selected from the group consisting of: SEQ ID NOs:2/10; 18/26; 34/42; 50/58; 66/58; 74/58; 82/58; 90/58; 98/58; 106/114; 122/130; 138/146; 154/162; 170/178; 186/194; 202/210; 218/226; 234/242; 250/258; 266/274; 282/290; 298/306; 314/322; 330/338; 346/354; 362/370; and 378/386.

According to another aspect, the present disclosure provides antibody-drug conjugates comprising an anti-STEAP2 antibody or antigen-binding fragment thereof as described above and a therapeutic agent (e.g., an anti-tumor agent, e.g., a camptothecin analog, e.g., Dxd). In some embodiments, the antibody or antigen-binding fragment and the anti-tumor agent are covalently attached via a linker, as discussed above. In various embodiments, the anti-STEAP2 antibody or antigen-binding fragment can be any of the anti-STEAP 2 antibodies or fragments described herein.

Heavy and Light Chain Variable Region Amino Acid and Nucleic Acid Sequences of anti-STEAP2 antibodies

Table 5 sets forth the amino acid sequence identifiers of the heavy and light chain variable regions and CDRs of selected anti-STEAP2 antibodies according to the disclosure. The corresponding nucleic acid sequence identifiers are set forth in Table 6.

TABLE 5
Amino Acid Sequence Identifiers of anti-STEAP2 antibodies

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H1H11243N | 2 | 4 | 6 | 8 | 10 | 12 | 14 | 16 |
| H1H11878P | 18 | 20 | 22 | 24 | 26 | 28 | 30 | 32 |
| H1H11880P | 34 | 36 | 38 | 40 | 42 | 44 | 46 | 48 |
| H1H11888P2 | 50 | 52 | 54 | 56 | 58 | 60 | 62 | 64 |
| H1H11892P2 | 66 | 68 | 70 | 72 | 58 | 60 | 62 | 64 |
| H1H11893P2 | 74 | 76 | 78 | 80 | 58 | 60 | 62 | 64 |
| H1H11894P2 | 82 | 84 | 86 | 88 | 58 | 60 | 62 | 64 |
| H1H11895P2 | 90 | 92 | 94 | 96 | 58 | 60 | 62 | 64 |
| H1H11896P2 | 98 | 100 | 102 | 104 | 58 | 60 | 62 | 64 |
| H1H11897P2 | 106 | 108 | 110 | 112 | 114 | 116 | 118 | 120 |
| H1H7968P | 122 | 124 | 126 | 128 | 130 | 132 | 134 | 136 |
| H1H7969P | 138 | 140 | 142 | 144 | 146 | 148 | 150 | 152 |
| H1H7970P | 154 | 156 | 158 | 160 | 162 | 164 | 166 | 168 |
| H1H7971P | 170 | 172 | 174 | 176 | 178 | 180 | 182 | 184 |
| H1H7972P | 186 | 188 | 190 | 192 | 194 | 196 | 198 | 200 |
| H1M7804N | 202 | 204 | 206 | 208 | 210 | 212 | 214 | 216 |
| H1M7814N | 218 | 220 | 222 | 224 | 226 | 228 | 230 | 232 |
| H1M7832N | 234 | 236 | 238 | 240 | 242 | 244 | 246 | 248 |
| H2M11162N | 250 | 252 | 254 | 256 | 258 | 260 | 262 | 264 |
| H2M11163N | 266 | 268 | 270 | 272 | 274 | 276 | 278 | 280 |
| H2M11164N | 282 | 284 | 286 | 288 | 290 | 292 | 294 | 296 |
| H2M7806N | 298 | 300 | 302 | 304 | 306 | 308 | 310 | 312 |
| H2M7807N | 314 | 316 | 318 | 320 | 322 | 324 | 326 | 328 |
| H2M7809N | 330 | 332 | 334 | 336 | 338 | 340 | 342 | 344 |
| H2M7810N | 346 | 348 | 350 | 352 | 354 | 356 | 358 | 360 |
| H2M7811N | 362 | 364 | 366 | 368 | 370 | 372 | 374 | 376 |
| H2M7812N | 378 | 380 | 382 | 384 | 386 | 388 | 390 | 392 |

TABLE 6
Nucleic Acid Sequence Identifiers of Anti-STEAP2 Antibodies

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H1H11243N | 1 | 3 | 5 | 7 | 9 | 11 | 13 | 15 |
| H1H11878P | 17 | 19 | 21 | 23 | 25 | 27 | 29 | 31 |
| H1H11880P | 33 | 35 | 37 | 39 | 41 | 43 | 45 | 47 |
| H1H11888P2 | 49 | 51 | 53 | 55 | 57 | 59 | 61 | 63 |
| H1H11892P2 | 65 | 67 | 69 | 71 | 57 | 59 | 61 | 63 |
| H1H11893P2 | 73 | 75 | 77 | 79 | 57 | 59 | 61 | 63 |
| H1H11894P2 | 81 | 83 | 85 | 87 | 57 | 59 | 61 | 63 |
| H1H11895P2 | 89 | 91 | 93 | 95 | 57 | 59 | 61 | 63 |
| H1H11896P2 | 97 | 99 | 101 | 103 | 57 | 59 | 61 | 63 |
| H1H11897P2 | 105 | 107 | 109 | 111 | 113 | 115 | 117 | 119 |
| H1H7968P | 121 | 123 | 125 | 127 | 129 | 131 | 133 | 135 |
| H1H7969P | 137 | 139 | 141 | 143 | 145 | 147 | 149 | 151 |
| H1H7970P | 153 | 155 | 157 | 159 | 161 | 163 | 165 | 167 |
| H1H7971P | 169 | 171 | 173 | 175 | 177 | 179 | 181 | 183 |
| H1H7972P | 185 | 187 | 189 | 191 | 193 | 195 | 197 | 199 |
| H1M7804N | 201 | 203 | 205 | 207 | 209 | 211 | 213 | 215 |
| H1M7814N | 217 | 219 | 221 | 223 | 225 | 227 | 229 | 23 |
| H1M7832N | 233 | 235 | 237 | 239 | 241 | 243 | 245 | 247 |
| H2M11162N | 249 | 251 | 253 | 255 | 257 | 259 | 261 | 263 |
| H2M11163N | 265 | 267 | 269 | 271 | 273 | 275 | 277 | 279 |
| H2M11164N | 281 | 283 | 285 | 287 | 289 | 291 | 293 | 295 |
| H2M7806N | 297 | 299 | 301 | 303 | 305 | 307 | 309 | 311 |
| H2M7807N | 313 | 315 | 317 | 319 | 321 | 323 | 325 | 327 |
| H2M7809N | 329 | 331 | 333 | 335 | 337 | 339 | 341 | 343 |
| H2M7810N | 345 | 347 | 349 | 351 | 353 | 355 | 357 | 359 |
| H2M7811N | 361 | 363 | 365 | 367 | 369 | 371 | 373 | 375 |

### Anti-MET Antibodies Suitable for Protein-Drug Conjugates

In some embodiments, the antibody is an anti MET antibody. According to certain embodiments, protein-drug conjugates, e.g., ADCs, according to the disclosure comprise anti-MET antibody. In some embodiment, the anti-MET antibody may include those described in US 2018/0134794.

In some embodiments, the antibody is an anti-MET/MET bispecific antibody, which comprises a first antigen-binding domain (D1) which specifically binds a first epitope of human MET and a second antigen-binding domain (D2) which specifically binds a second epitope of human MET. In some embodiment, the anti-MET/MET bispecific antibody may include those described in US 2018/0134794.

In certain embodiments, D1 and D2 domains of an anti-MET/MET bispecific antibody are non-competitive with one another. Non-competition between D1 and D2 for binding to MET means that, the respective monospecific antigen binding proteins from which D1 and D2 were derived do not compete with one another for binding to human MET. Exemplary antigen-binding protein competition assays are known in the art.

In certain embodiments, D1 and D2 bind to different (e.g., non-overlapping, or partially overlapping) epitopes on MET.

In one non-limiting embodiment, the present disclosure provides protein-drug conjugates comprising a bispecific antigen-binding molecule comprising:
a first antigen-binding domain (D1); and
a second antigen-binding domain (D2);
wherein D1 specifically binds a first epitope of human MET; and
wherein D2 specifically binds a second epitope of human MET.

Anti-MET/MET bispecific antibodies may be constructed using the antigen-binding domains of two separate monospecific anti-MET antibodies. For example, a collection of monoclonal monospecific anti-MET antibodies may be produced using standard methods known in the art. The individual antibodies thus produced may be tested pairwise against one another for cross-competition to a MET protein. If two different anti-MET antibodies are able to bind to MET at the same time (i.e., do not compete with one another), then the antigen-binding domain from the first anti-MET antibody and the antigen-binding domain from the second, non-competitive anti-MET antibody can be engineered into a single anti-MET/MET bispecific antibody in accordance with the present disclosure.

According to the present disclosure, a bispecific antigen-binding molecule can be a single multifunctional polypeptide, or it can be a multimeric complex of two or more polypeptides that are covalently or non-covalently associated with one another. As will be made evident by the present disclosure, any antigen binding construct which has the ability to simultaneously bind two separate, non-identical epitopes of the MET molecule is regarded as a bispecific antigen-binding molecule. Any of the bispecific antigen-binding molecules described herein, or variants thereof, may be constructed using standard molecular biological techniques (e.g., recombinant DNA and protein expression technology) as will be known to a person of ordinary skill in the art.

The bispecific antigen-binding molecules, which comprise a first antigen-binding domain (D1) which specifically binds a first epitope of human MET and a second antigen-binding domain (D2) which specifically binds a second epitope of human MET, may be referred to herein as “MET/MET bispecific antibodies,” “MET×MET bispecific antibodies,” “MET/MET,” “MET×MET” or other related terminology. In some embodiments, the first epitope of human MET comprises amino acids 192-204 of SEQ ID NO:2109. In some embodiments, the second epitope of human MET comprises amino acids 305-315 and 421-455 of SEQ ID NO:2109. In some embodiments, the first epitope of human MET comprises amino acids 192-204 of SEQ ID NO:2109; and the second epitope of human MET comprises amino acids 305-315 and 421-455 of SEQ ID NO:2109.

Exemplary antigen-binding domains (D1 and D2) that can be included in the MET×MET bispecific antigen-binding molecules provided herein include antigen-binding domains derived from any of the anti-MET antibodies disclosed herein. For example, the present disclosure includes MET×MET bispecific antigen-binding molecules comprising a D1 or D2 antigen-binding domain comprising an HCVR comprising an amino acid sequence selected from any of the HCVR amino acid sequences listed in Table 7, or a substantially similar sequence thereof having at least 90%, at least 95%, at least 98% or at least 99% sequence identity thereto.

Also provided herein are MET×MET bispecific antigen-binding molecules comprising a D1 or D2 antigen-binding domain comprising an LCVR comprising an amino acid sequence selected from any of the LCVR amino acid sequences listed in Table 7, or a substantially similar sequence thereof having at least 90%, at least 95%, at least 98% or at least 99% sequence identity thereto.

Provided herein are MET×MET bispecific antigen-binding molecules comprising a D1 or D2 antigen-binding domain comprising an HCVR and an LCVR amino acid sequence pair (HCVR/LCVR) comprising any of the HCVR amino acid sequences listed in Table 7 paired with any of the LCVR amino acid sequences listed in Table 7. According to certain embodiments, the present invention provides MET×MET bispecific antigen-binding molecules comprising a D1 or D2 antigen-binding domain comprising an HCVR/LCVR amino acid sequence pair contained within any of the exemplary anti-MET antibodies listed in Table 7.

Also provided herein are MET×MET bispecific antigen-binding molecules comprising a D1 or D2 antigen-binding domain comprising a heavy chain CDR1 (HCDR1) comprising an amino acid sequence selected from any of the HCDR1 amino acid sequences listed in Table 7 or a substantially similar sequence thereof having at least 90%, at least 95%, at least 98% or at least 99% sequence identity.

Also provided are MET×MET bispecific antigen-binding molecules comprising a D1 or D2 antigen-binding domain comprising a heavy chain CDR2 (HCDR2) comprising an amino acid sequence selected from any of the HCDR2 amino acid sequences listed in Table 7 or a substantially similar sequence thereof having at least 90%, at least 95%, at least 98% or at least 99% sequence identity.

Also provided are MET×MET bispecific antigen-binding molecules comprising a D1 or D2 antigen-binding domain comprising a heavy chain CDR3 (HCDR3) comprising an amino acid sequence selected from any of the HCDR3 amino acid sequences listed in Table 7 or a substantially similar sequence thereof having at least 90%, at least 95%, at least 98% or at least 99% sequence identity.

Also provided are MET×MET bispecific antigen-binding molecules comprising a D1 or D2 antigen-binding domain comprising a light chain CDR1 (LCDR1) comprising an amino acid sequence selected from any of the LCDR1 amino acid sequences listed in Table 7 or a substantially similar sequence thereof having at least 90%, at least 95%, at least 98% or at least 99% sequence identity.

Also provided are MET×MET bispecific antigen-binding molecules comprising a D1 or D2 antigen-binding domain comprising a light chain CDR2 (LCDR2) comprising an amino acid sequence selected from any of the LCDR2 amino acid sequences listed in Table 7 or a substantially similar sequence thereof having at least 90%, at least 95%, at least 98% or at least 99% sequence identity.

Also provided are MET×MET bispecific antigen-binding molecules comprising a D1 or D2 antigen-binding domain comprising a light chain CDR3 (LCDR3) comprising an amino acid sequence selected from any of the LCDR3 amino acid sequences listed in Table 7 or a substantially similar sequence thereof having at least 90%, at least 95%, at least 98% or at least 99% sequence identity.

Also provided are MET×MET bispecific antigen-binding molecules comprising a D1 or D2 antigen-binding domain comprising an HCDR3 and an LCDR3 amino acid sequence pair (HCDR3/LCDR3) comprising any of the HCDR3 amino acid sequences listed in Table 7 paired with any of the LCDR3 amino acid sequences listed in Table 7. According to certain embodiments, the present disclosure provides antibodies, or antigen-binding fragments thereof, comprising an HCDR3/LCDR3 amino acid sequence pair contained within any of the exemplary anti-MET antibodies listed in Table 7.

Also provided are MET×MET bispecific antigen-binding molecules comprising a D1 or D2 antigen-binding domain comprising a set of six CDRs (i.e., HCDR1-HCDR2-HCDR3-LCDR1-LCDR2-LCDR3) contained within any of the exemplary anti-MET antibodies listed in Table 7.

In a related embodiment, the present disclosure provides MET×MET bispecific antigen-binding molecules comprising a D1 or D2 antigen-binding domain comprising a set of six CDRs (i.e., HCDR1-HCDR2-HCDR3-LCDR1-LCDR2-LCDR3) contained within an HCVR/LCVR amino acid sequence pair as defined by any of the exemplary anti-MET antibodies listed in Table 7.

The MET×MET bispecific antigen-binding molecules provided herein may comprise a D1 antigen-binding domain derived from any of the anti-MET antibodies of Table 7, and a D2 antigen-binding domain derived from any other anti-MET antibody of Table 7.

As a non-limiting illustrative example, the present disclosure includes MET×MET bispecific antigen binding molecules comprising a D1 antigen-binding domain and a D2 antigen-binding domain, wherein the D1 antigen binding domain comprises an HCVR/LCVR amino acid sequence pair of SEQ ID NOs: 2012/2092, or a set of heavy and light chain CDRs (HCDR1-HCDR2-HCDR3-LCDR1-LCDR2-LCDR3) comprising SEQ ID NOs: 2014-2016-2018-2094-2096-2098, and wherein the D2 antigen-binding domain comprises an HCVR/LCVR amino acid sequence pair of SEQ ID NOs: 2036/2092, or a set of heavy and light chain CDRs (HCDR1-HCDR2-HCDR3-LCDR1-LCDR2-LCDR3) comprising SEQ ID NOs: 2038-2040-2042-2094-2096-2098. An exemplary MET×MET bispecific antibody having these sequence characteristics is the bispecific antibody designated H4H14639D, also referred to as bispecific antibody No. 2076, which comprises a D1 derived from H4H13306P2 and a D2 derived from H4H13312P2.

Heavy and Light Chain Variable Region Amino Acid and Nucleic Acid Sequences for anti-MET and MET/MET antibodies

Table 7 sets forth the amino acid sequence identifiers of the heavy and light chain variable regions and CDRs of selected anti-MET antibodies described herein. (As noted above, all anti-MET antibodies of the present disclosure possess the same light chain variable region, and thus the same light chain CDR sequences as well). The corresponding nucleic acid sequence identifiers are set forth in Table 8.

TABLE 7
Amino Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H4H13290P2 | 1956 | 1958 | 1960 | 1962 | 2092 | 2094 | 2096 | 2098 |
| H4H13291P2 | 1964 | 1966 | 1968 | 1970 | 2092 | 2094 | 2096 | 2098 |
| H4H13295P2 | 1972 | 1974 | 1976 | 1978 | 2092 | 2094 | 2096 | 2098 |
| H4H13299P2 | 1980 | 1982 | 1984 | 1986 | 2092 | 2094 | 2096 | 2098 |
| H4H13300P2 | 1988 | 1990 | 1992 | 1994 | 2092 | 2094 | 2096 | 2098 |
| H4H13301P2 | 1996 | 1998 | 2000 | 2002 | 2092 | 2094 | 2096 | 2098 |
| H4H13302P2 | 2004 | 2006 | 2008 | 2010 | 2092 | 2094 | 2096 | 2098 |
| H4H13306P2 | 2012 | 2014 | 2016 | 2018 | 2092 | 2094 | 2096 | 2098 |
| H4H13309P2 | 2020 | 2022 | 2024 | 2026 | 2092 | 2094 | 2096 | 2098 |
| H4H13311P2 | 2028 | 2030 | 2032 | 2034 | 2092 | 2094 | 2096 | 2098 |
| H4H13312P2 | 2036 | 2038 | 2040 | 2042 | 2092 | 2094 | 2096 | 2098 |
| H4H13313P2 | 2044 | 2046 | 2048 | 2050 | 2092 | 2094 | 2096 | 2098 |
| H4H13316P2 | 2052 | 2054 | 2056 | 2058 | 2092 | 2094 | 2096 | 2098 |
| H4H13318P2 | 2060 | 2062 | 2064 | 2066 | 2092 | 2094 | 2096 | 2098 |
| H4H13319P2 | 2068 | 2070 | 2072 | 2074 | 2092 | 2094 | 2096 | 2098 |
| H4H13325P2 | 2076 | 2078 | 2080 | 2082 | 2092 | 2094 | 2096 | 2098 |
| H4H13331P2 | 2084 | 2086 | 2088 | 2090 | 2092 | 2094 | 2096 | 2098 |

TABLE 8
Nucleic Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H4H13290P2 | 1955 | 1957 | 1959 | 1961 | 2091 | 2093 | 2095 | 2097 |
| H4H13291P2 | 1963 | 1965 | 1967 | 1969 | 2091 | 2093 | 2095 | 2097 |
| H4H13295P2 | 1971 | 1973 | 1975 | 1977 | 2091 | 2093 | 2095 | 2097 |
| H4H13299P2 | 1979 | 1981 | 1983 | 1985 | 2091 | 2093 | 2095 | 2097 |
| H4H13300P2 | 1987 | 1989 | 1991 | 1993 | 2091 | 2093 | 2095 | 2097 |
| H4H13301P2 | 1995 | 1997 | 1999 | 2001 | 2091 | 2093 | 2095 | 2097 |
| H4H13302P2 | 2003 | 2005 | 2007 | 2009 | 2091 | 2093 | 2095 | 2097 |
| H4H13306P2 | 2011 | 2013 | 2015 | 2017 | 2091 | 2093 | 2095 | 2097 |
| H4H13309P2 | 2019 | 2021 | 2023 | 2025 | 2091 | 2093 | 2095 | 2097 |
| H4H13311P2 | 2027 | 2029 | 2031 | 2033 | 2091 | 2093 | 2095 | 2097 |
| H4H13312P2 | 2035 | 2037 | 2039 | 2041 | 2091 | 2093 | 2095 | 2097 |
| H4H13313P2 | 2043 | 2045 | 2047 | 2049 | 2091 | 2093 | 2095 | 2097 |
| H4H13316P2 | 2051 | 2053 | 2055 | 2057 | 2091 | 2093 | 2095 | 2097 |
| H4H13318P2 | 2059 | 2061 | 2063 | 2065 | 2091 | 2093 | 2095 | 2097 |
| H4H13319P2 | 2067 | 2069 | 2071 | 2073 | 2091 | 2093 | 2095 | 2097 |
| H4H13325P2 | 2075 | 2077 | 2079 | 2081 | 2091 | 2093 | 2095 | 2097 |
| H4H13331P2 | 2083 | 2085 | 2087 | 2089 | 2091 | 2093 | 2095 | 2097 |

Antibodies are typically referred to herein according to the following nomenclature: Fc prefix (e.g. “H-4H-”), followed by a numerical identifier (e.g. “13290,” “13291,” “13295,” etc.), followed by a “P2” suffix, as shown in Tables 7 and 8. Thus, according to this nomenclature, an antibody may be referred to herein as, e.g., “H41H13290P2,” “H4H13291P2,” “H41H13295P2,” etc. The prefix on the antibody designations used herein indicate the particular Fc region isotype of the antibody. In particular, an “H4H” antibody has a human IgG4 Fc (all variable regions are fully human as denoted by the first ‘H’ in the antibody designation). As will be appreciated by a person of ordinary skill in the art, an antibody having a particular Fc isotype can be converted to an antibody with a different Fc isotype (e.g., an antibody with a mouse IgG4 Fc can be converted to an antibody with a human IgG1, etc.), but in any event, the variable domains (including the CDRs)—which are indicated by the numerical identifiers shown in Tables 7 and 8—will remain the same, and the binding properties are expected to be identical or substantially similar regardless of the nature of the Fc domain.

### Antibody Conjugation

Techniques and linkers for conjugating to residues of an antibody or antigen binding fragment are known in the art. Exemplary amino acid attachments that can be used in the context of this aspect, e.g., lysine (see, e.g., U.S. Pat. No. 5,208,020; US 2010/0129314; Hollander et al., Bioconjugate Chem., 2008, 19:358-361; WO 2005/089808; U.S. Pat. No. 5,714,586; US 2013/0101546; and US 2012/0585592), cysteine (see, e.g., US 2007/0258987; WO 2013/055993; WO 2013/055990; WO 2013/053873; WO 2013/053872; WO 2011/130598; US 2013/0101546; and U.S. Pat. No. 7,750,116), selenoysteine (see, e.g., WO 2008/122039; and Hofer et al., Proc. Natl. Acad. Sci., USA, 2008, 105:12451-12456), formyl glycine (see, e.g., Carrico et al., Nat. Chem. Biol., 2007, 3:321-322; Agarwal et al., Proc. Natl. Acad. Sci., USA, 2013, 110:46-51, and Rabuka et al., Nat. Protocols, 2012, 10:1052-1067), non-natural amino acids (see, e.g., WO 2013/068874, and WO 2012/166559), and acidic amino acids (see, e.g., WO 2012/05982). Lysine conjugation can also proceed through NHS (N-hydroxy succinimide). Linkers can also be conjugated to cysteine residues, including cysteine residues of a cleaved interchain disulfide bond, by forming a carbon bridge between thiols (see, e.g., U.S. Pat. Nos. 9,951,141, and 9,950,076). Linkers can also be conjugated to an antigen-binding protein via attachment to carbohydrates (see, e.g., US 2008/0305497, WO 2014/065661, and Ryan et al., Food & Agriculture Immunol., 2001, 13:127-130) and disulfide linkers (see, e.g., WO 2013/085925, WO 2010/010324, WO 2011/018611, and Shaunak et al., Nat. Chem. Biol., 2006, 2:312-313). Site specific conjugation techniques can also be employed to direct conjugation to particular residues of the antibody or antigen binding protein (see, e.g., Schumacher et al. J Clin Immunol (2016) 36 (Suppl 1): 100). In specific embodiments discussed in more detail below, Site specific conjugation techniques, include glutamine conjugation via transglutaminase (see e.g., Schibli, Angew Chemie Inter Ed. 2010, 49,9995).

Payloads according to the disclosure linked through lysine and/or cysteine, e.g., via a maleimide or amide conjugation, are included within the scope of the present disclosure.

In some embodiments, the protein-drug conjugates of the present disclosure are produced according to a two-step process, where Step 1 is lysine-based linker conjugation, e.g., with an NHS-ester linker, and Step 2 is a payload conjugation reaction (e.g., a 1,3-cycloaddition reaction).

In some embodiments, the protein-drug conjugates of the present disclosure are produced according to a two-step process, where Step 1 is cysteine-based linker conjugation, e.g., with a maleimide linker, and Step 2 is a payload conjugation reaction (e.g., a 1,3-cycloaddition reaction).

In some embodiments, the protein-drug conjugates of the present disclosure are produced according to a two-step process, where Step 1 is transglutaminase-mediated site specific conjugation and Step 2 is a payload conjugation reaction (e.g., a 1,3-cycloaddition reaction).

### Step 1: Transglutaminase Mediated Site Specific Conjugation

In some embodiments, proteins (e.g., antibodies) may be modified in accordance with known methods to provide glutaminyl modified proteins. Techniques for conjugating antibodies and primary amine compounds are known in the art. Site specific conjugation techniques are employed herein to direct conjugation to glutamine using glutamine conjugation via transglutaminase (see e.g., Schibli, Angew Chemie Inter Ed. 2010, 49, 9995).

Primary amine-comprising compounds (e.g., linkers L1) of the present disclosure can be conjugated to one or more glutamine residues of a binding agent (e.g., a protein, e.g., an antibody) via transglutaminase-based chemo-enzymatic conjugation (see, e.g., Dennler et al., Protein Conjugate Chem. 2014, 25, 569-578, and WO 2017/147542). For example, in the presence of transglutaminase, one or more glutamine residues of an antibody can be coupled to a primary amine linker compound. Briefly, in some embodiments, a binding agent having a glutamine residue (e.g., a gln295, i.e. Q295 residue) is treated with a primary amine-containing linker LL, described above, in the presence of the enzyme transglutaminase. In certain embodiments, the binding agent is aglycosylated. In certain embodiments, the binding agent is deglycosylated.

In certain embodiments, the binding agent (e.g., a protein, e.g., an antibody) comprises at least one glutamine residue in at least one polypeptide chain sequence. In certain embodiments, the binding agent comprises two heavy chain polypeptides, each with one gln295 residue. In further embodiments, the binding agent comprises one or more glutamine residues at a site other than a heavy chain 295.

In some embodiments, a binding agent, such as an antibody, can be prepared by site-directed mutagenesis to insert a glutamine residue at a site without resulting in disabled antibody function or binding. For example, included herein are antibodies bearing Asn297Gln (N297Q) mutation(s) as described herein. In some embodiments, an antibody having a gln295 residue and/or an N297Q mutation contains one or more additional naturally occurring glutamine residues in their variable regions, which can be accessible to transglutaminase and therefore capable of conjugation to a linker or a linker-payload. An exemplary naturally occurring glutamine residue can be found, e.g., at Q55 of the light chain. In such instances, the binding agent, e.g., antibody, conjugated via transglutaminase can have a higher than expected LAR value (e.g., a LAR higher than 4). Any such antibodies can be isolated from natural or artificial sources.

In certain embodiments of the disclosure, the linker-antibody ratio or LAR is from 1, 2, 3, 4, 5, 6, 7, or 8 linker LL molecules per antibody. In some embodiments, the LAR is from 1 to 8. In some embodiments, the LAR is from 1 to 6. In certain embodiments, the LAR is from 2 to 4. In some cases, the LAR is from 2 to 3. In certain cases, the LAR is from 0.5 to 3.5. In some embodiments, the LAR is about 1, or about 1.5, or about 2, or about 2.5, or about 3, or about 3.5. In some embodiments, the LAR is 2. In some embodiments, the LAR is 4.

### Step 2: Payload Conjugation Reaction

In certain embodiments, linkers LL according to the present disclosure comprise at least one reactive group capable of further reaction after transglutamination. In these embodiments, the glutaminyl-modified protein (e.g., antibody) is capable of further reaction with a reactive payload compound or a reactive linker-payload compound (e.g., linker-payload compounds as disclosed herein), to form a protein-payload conjugate. More specifically, the reactive linker-payload compound may comprise a reactive group that is capable of reacting with the reactive group of the linker LL via a click chemistry reaction to form a click chemistry adduct. In certain embodiments, a reactive group according to the present disclosure comprises a moiety that is capable of undergoing a 1,3-cycloaddition reaction. In certain embodiments, the reactive group is an azide. In certain embodiments, the reactive group comprises an alkyne (e.g., a terminal alkyne, or an internal strained alkyne). In certain embodiments, the reactive group comprises a tetrazine. In certain embodiments, the reactive group comprises a strained alkene. In certain embodiments of the present disclosure the reactive group is compatible with the binding agent and transglutamination reaction conditions.

In one embodiment, the glutamine residue Gln is naturally present in a CH2 or CH3 domain of the BA. In another embodiment, the glutamine residue Gln is introduced to the BA by modifying one or more amino acids. In one embodiment, the Gln is Q295 or N297Q.

In one embodiment, the transglutaminase is microbial transglutaminase (MTG). In one embodiment, the transglutaminase is bacterial transglutaminase (BTG).

### Anti-HER2 Antibody-Drug Conjugates

In certain embodiments, the protein-drug conjugates, e.g., ADCs, disclosed herein are useful, inter alia, for the treatment, prevention and/or amelioration of any disease or disorder associated with or mediated by HER2 expression or activity, or treatable by binding HER2 without competing against modified LDL, or and/or promoting HER2 receptor internalization and/or decreasing cell surface receptor number.

The protein-drug conjugates of the present disclosure (and therapeutic compositions comprising the same) are useful, inter alia, for treating any disease or disorder in which stimulation, activation and/or targeting of an immune response would be beneficial. In particular, the anti-HER2 protein-drug conjugates, including both monospecific anti-HER2 antibodies and bispecific anti-HER2/HER2 antibodies of the present disclosure can be used for the treatment, prevention and/or amelioration of any disease or disorder associated with or mediated by HER2 expression or activity or the proliferation of HER2+ cells. The mechanism of action by which the therapeutic methods of the present disclosure are achieved include killing of the cells expressing HER2 in the presence of effector cells, for example, by CDC, apoptosis, ADCC, phagocytosis, or by a combination of two or more of these mechanisms. Cells expressing HER2 which can be inhibited or killed using the protein-drug conjugates of the present disclosure include, for example, breast tumor cells.

In one embodiment, the protein-drug conjugates of the present disclosure (and therapeutic compositions and dosage forms comprising same) comprise a bispecific antigen-binding molecule comprising:
a first antigen-binding domain (D1); and
a second antigen-binding domain (D2);
wherein D1 specifically binds a first epitope of human HER2; and
wherein D2 specifically binds a second epitope of human HER2.

In one embodiment of the above, D1 and D2 do not compete with one another for binding to human HER2.

The protein-drug conjugates of the present disclosure can be used to treat, e.g., primary and/or metastatic tumors arising in the prostate, bladder, cervix, lung, colon, kidney, breast, pancreas, stomach, uterus, and/or ovary. In certain embodiments, the protein-drug conjugates of the present disclosure are used to treat one or more of the following cancers: prostate cancer, bladder cancer, cervical cancer, lung cancer, colon cancer, kidney cancer, breast cancer, pancreatic cancer, stomach cancer, uterine cancer, and ovarian cancer. According to certain embodiments of the present disclosure, the anti-HER2 antibodies or anti-HER2/HER2 bispecific antibodies are useful for treating a patient afflicted with a breast cancer cell that is IHC2+ or more. According to other related embodiments of the present disclosure, methods are provided comprising administering an anti-HER2 antibody or an anti-HER2/HER2 antibody as disclosed herein to a patient who is afflicted with a breast cancer cell that is IHC2+ or more. Analytic/diagnostic methods known in the art, such as tumor scanning, etc., can be used to ascertain whether a patient harbors a tumor that is castrate-resistant.

In certain embodiments, the present disclosure also includes methods for treating residual cancer in a subject. The term “residual cancer” means the existence or persistence of one or more cancerous cells in a subject following treatment with an anti-cancer therapy.

The protein-drug conjugates of the present disclosure (and therapeutic compositions comprising the same) are useful, inter alia, for treating any disease or disorder in which stimulation, activation and/or targeting of an immune response would be beneficial. In particular, protein-drug conjugates comprising the anti-HER2 antibodies or anti HER2/HER2 antibodies of the present disclosure can be used for the treatment, prevention and/or amelioration of any disease or disorder associated with or mediated by HER2 expression or activity or the proliferation of HER2+ cells. The mechanism of action by which the therapeutic methods of the present disclosure are achieved include killing of the cells expressing HER2 in the presence of effector cells, for example, by CDC, apoptosis, ADCC, phagocytosis, or by a combination of two or more of these mechanisms. Cells expressing HER2 which can be inhibited or killed using the protein-drug conjugates of the present disclosure include, for example, breast tumor cells.

According to certain aspects, the present disclosure provides methods for treating a disease or disorder associated with HER2 expression (e.g., breast cancer) comprising administering one or more of the anti-HER2 protein-drug conjugates or anti-HER2/HER2 bispecific protein-drug conjugates described elsewhere herein to a subject after the subject has been determined to have breast cancer (e.g., and IHC2+ breast cancer). For example, the present disclosure includes methods for treating breast cancer comprising administering protein-drug conjugate comprising an anti-HER2 antibody or antigen-binding molecule or an anti-HER2/HER2 bispecific antibody or antigen-binding molecule to a patient 1 day, 2 days, 3 days, 4 days, 5 days, 6 days, 1 week, 2 weeks, 3 weeks or 4 weeks, 2 months, 4 months, 6 months, 8 months, 1 year, or more after the subject has received hormone therapy (e.g., anti-androgen therapy).

In certain embodiments, the present disclosure also includes the use of an anti-HER2 antibody of the present disclosure in the manufacture of a medicament for the treatment of a disease or disorder (e.g., cancer) related to or caused by HER2-expressing cells. In one aspect, the present disclosure relates to a protein-drug conjugate comprising an anti-HER2 antibody or antigen-binding fragment or an anti-HER2/HER2 bispecific antibody or antigen-binding fragment, as disclosed herein, for use in medicine. In one aspect, the present disclosure relates to a compound comprising an antibody-drug conjugate (ADC) as disclosed herein, for use in medicine.

### Anti-STEAP2 Antibody-Drug Conjugates

In certain embodiments, the protein-drug conjugates, e.g., ADCs, disclosed herein are useful, inter alia, for the treatment, prevention and/or amelioration of any disease or disorder associated with or mediated by STEAP2 expression or activity, or treatable by binding STEAP2 without competing against modified LDL, or and/or promoting STEAP2 receptor internalization and/or decreasing cell surface receptor number.

The protein-drug conjugates of the present disclosure (and therapeutic compositions comprising the same) are useful, inter alia, for treating any disease or disorder in which stimulation, activation and/or targeting of an immune response would be beneficial. In particular, the anti-STEAP2 protein-drug conjugates of the present disclosure can be used for the treatment, prevention and/or amelioration of any disease or disorder associated with or mediated by STEAP2 expression or activity or the proliferation of STEAP2+ cells. The mechanism of action by which the therapeutic methods of the present disclosure are achieved include killing of the cells expressing STEAP2 in the presence of effector cells, for example, by CDC, apoptosis, ADCC, phagocytosis, or by a combination of two or more of these mechanisms. Cells expressing STEAP2 which can be inhibited or killed using the protein-drug conjugates of the present disclosure include, for example, prostate tumor cells.

The protein-drug conjugates of the present disclosure can be used to treat, e.g., primary and/or metastatic tumors arising in the prostate, bladder, cervix, lung, colon, kidney, breast, pancreas, stomach, uterus, and/or ovary. In certain embodiments, the protein-drug conjugates of the present disclosure are used to treat one or more of the following cancers: prostate cancer, bladder cancer, cervical cancer, lung cancer, colon cancer, kidney cancer, breast cancer, pancreatic cancer, stomach cancer, uterine cancer, and ovarian cancer. Analytic/diagnostic methods known in the art, such as tumor scanning, etc., can be used to ascertain whether a patient harbors a tumor that is castrate-resistant.

In certain embodiments, the present disclosure also includes methods for treating residual cancer in a subject. The term “residual cancer” means the existence or persistence of one or more cancerous cells in a subject following treatment with an anti-cancer therapy.

According to certain aspects, the present disclosure provides methods for treating a disease or disorder associated with STEAP2 expression (e.g., prostate cancer) comprising administering one or more of the anti-STEAP2 protein-drug conjugates described elsewhere herein to a subject after the subject has been determined to have prostate cancer. For example, the present disclosure includes methods for treating prostate cancer comprising administering protein-drug conjugate comprising an anti-STEAP2 antibody or antigen-binding molecule to a patient 1 day, 2 days, 3 days, 4 days, 5 days, 6 days, 1 week, 2 weeks, 3 weeks or 4 weeks, 2 months, 4 months, 6 months, 8 months, 1 year, or more after the subject has received hormone therapy (e.g., anti-androgen therapy).

In certain embodiments, the present disclosure also includes the use of an anti-STEAP2 antibody of the present disclosure in the manufacture of a medicament for the treatment of a disease or disorder (e.g., cancer) related to or caused by STEAP2-expressing cells. In one aspect, the present disclosure relates to a protein-drug conjugate comprising an anti-STEAP2 antibody or antigen-binding fragment, as disclosed herein, for use in medicine. In one aspect, the present disclosure relates to a compound comprising an antibody-drug conjugate (ADC) as disclosed herein, for use in medicine.

### Anti-MET Antibody-Drug Conjugates

In certain embodiments, the protein-drug conjugates, e.g., ADCs, disclosed herein are useful, inter alia, for the treatment, prevention and/or amelioration of any disease or disorder associated with or mediated by MET expression or activity, or treatable by binding MET without competing against modified LDL, or and/or promoting MET receptor internalization and/or decreasing cell surface receptor number.

The protein-drug conjugates of the present disclosure (and therapeutic compositions comprising the same) are useful, inter alia, for treating any disease or disorder in which stimulation, activation and/or targeting of an immune response would be beneficial. In particular, the anti-MET or anti MET/MET bispecific protein-drug conjugates of the present disclosure can be used for the treatment, prevention and/or amelioration of any disease or disorder associated with or mediated by MET expression or activity or the proliferation of MET+ cells. The mechanism of action by which the therapeutic methods of the present disclosure are achieved include killing of the cells expressing MET in the presence of effector cells, for example, by CDC, apoptosis, ADCC, phagocytosis, or by a combination of two or more of these mechanisms. Cells expressing MET which can be inhibited or killed using the protein-drug conjugates of the present disclosure include, for example, lung tumor cells.

The protein-drug conjugates of the present disclosure can be used to treat, e.g., primary and/or metastatic tumors arising in the prostate, bladder, cervix, lung, colon, kidney, breast, pancreas, stomach, uterus, and/or ovary. In certain embodiments, the protein-drug conjugates of the present disclosure are used to treat one or more of the following cancers: prostate cancer, bladder cancer, cervical cancer, lung cancer, colon cancer, kidney cancer, breast cancer, pancreatic cancer, stomach cancer, uterine cancer, and ovarian cancer. Analytic/diagnostic methods known in the art, such as tumor scanning, etc., can be used to ascertain whether a patient harbors a tumor that is castrate-resistant.

In certain embodiments, the present disclosure also includes methods for treating residual cancer in a subject. The term “residual cancer” means the existence or persistence of one or more cancerous cells in a subject following treatment with an anti-cancer therapy.

According to certain aspects, the present disclosure provides methods for treating a disease or disorder associated with MET expression (e.g., lung cancer) comprising administering one or more of the anti-MET or anti MET/MET bispecific protein-drug conjugates described elsewhere herein to a subject after the subject has been determined to have lung cancer. For example, the present disclosure includes methods for treating lung cancer comprising administering protein-drug conjugate comprising an anti-MET or anti MET/MET bispecific antibody or antigen-binding molecule to a patient 1 day, 2 days, 3 days, 4 days, 5 days, 6 days, 1 week, 2 weeks, 3 weeks or 4 weeks, 2 months, 4 months, 6 months, 8 months, 1 year, or more after the subject has received hormone therapy (e.g., anti-androgen therapy).

For example, anti-MET antibody-drug conjugates and MET×MET bispecific antibody-drug conjugates of the present disclosure are useful for the treatment of tumors that express (or overexpress) MET. For example, the anti-MET antibody-drug conjugates and MET×MET bispecific antibody-drug conjugates may be used to treat primary and/or metastatic tumors arising in the brain and meninges, oropharynx, lung and bronchial tree, gastrointestinal tract, male and female reproductive tract, muscle, bone, skin and appendages, connective tissue, spleen, immune system, blood forming cells and bone marrow, liver and urinary tract, and special sensory organs such as the eye. In certain embodiments, the anti-MET antibody-drug conjugates and MET×MET bispecific antibody-drug conjugates are used to treat one or more of the following cancers: acute myelogenous leukemia, adult T-cell leukemia, astrocytomas, bladder cancer, breast cancer, cervical cancer, cholangiocarcinoma, chronic myeloid leukemia, colorectal cancer, endometrial cancer, esophageal cancer, gastric cancer (e.g., gastric cancer with MET amplification), glioblastomata, head and neck cancer (e.g., head and neck squamous cell carcinoma [HNSCC]), Kaposi's sarcoma, kidney cancer, leiomyosarcomas, liver cancer, lung cancer (e.g., non-small cell lung cancer [NSCLC]), lymphomas, malignant gliomas, malignant mesothelioma, melanoma, mesothelioma, MFH/fibrosarcoma, multiple myeloma, nasopharyngeal cancer, osteosarcoma, ovarian cancer, pancreatic carcinoma, prostate cancer, renal cell carcinoma, rhabdomyosarcoma, small cell lung cancer, synovial sarcoma, thyroid cancer, and Wilms' tumor.

In certain embodiments, the present disclosure also includes the use of an anti-MET antibody-drug conjugate or a MET×MET bispecific antibody-drug conjugate of the present disclosure in the manufacture of a medicament for the treatment of a disease or disorder (e.g., cancer) related to or caused by MET-expressing cells. In one aspect, the present disclosure relates to a protein-drug conjugate comprising an anti-MET antibody-drug conjugate or a MET×MET bispecific antibody-drug conjugate, as disclosed herein, for use in medicine. In one aspect, the present disclosure relates to a compound comprising an antibody-drug conjugate (ADC) as disclosed herein, for use in medicine.

### Combination Therapies and Formulations

The present disclosure provides methods which comprise administering a pharmaceutical composition comprising any of the exemplary protein-drug conjugates (e.g., antibody-drug conjugates), linker-payloads and payloads described herein in combination with one or more additional therapeutic agents. Exemplary additional therapeutic agents that may be combined with or administered in combination with protein-drug conjugates (e.g., antibody-drug conjugates), linker-payloads and payloads of the present disclosure include, e.g., a HER2 antagonist (e.g., an anti-HER2 antibody [e.g., trastuzumab] or a small molecule inhibitor of HER2 or an anti-HER2 antibody-drug conjugate, or an anti-HER2/HER2 bispecific antibody or an anti-HER2/HER2 bispecific antibody-drug conjugate), an EGFR antagonist (e.g., an anti-EGFR antibody [e.g., cetuximab or panitumumab] or small molecule inhibitor of EGFR [e.g., gefitinib or erlotinib]), an antagonist of another EGFR family member such as HER2/ErbB2, ErbB3 or ErbB4 (e.g., anti-ErbB2, anti-ErbB3 or anti-ErbB4 antibody or small molecule inhibitor of ErbB2, ErbB3 or ErbB4 activity), an antagonist of EGFRvIII (e.g., an antibody that specifically binds EGFRvIII), a cMET antagonist (e.g., an anti-cMET antibody), an IGF1R antagonist (e.g., an anti-IGF1R antibody), a B-raf inhibitor (e.g., vemurafenib, sorafenib, gDC-0879, PLX-4720), a PDGFR-α inhibitor (e.g., an anti-PDGFR-α antibody), a PDGFR-β inhibitor (e.g., an anti-PDGFR-β antibody), a VEGF antagonist (e.g., a VEGF-Trap, see, e.g., U.S. Pat. No. 7,087,411 (also referred to herein as a “VEGF-inhibiting fusion protein”), anti-VEGF antibody (e.g., bevacizumab), a small molecule kinase inhibitor of VEGF receptor (e.g., sunitinib, sorafenib or pazopanib)), a DLL4 antagonist (e.g., an anti-DLL4 antibody disclosed in US 2009/0142354), an Ang2 antagonist (e.g., an anti-Ang2 antibody disclosed in US 2011/0027286 such as H1H685P), a FOLH1 (PSMA) antagonist, a PRLR antagonist (e.g., an anti-PRLR antibody), a STEAP1 or STEAP2 antagonist (e.g., an anti-STEAP1 antibody or an anti-STEAP2 antibody), a TMPRSS2 antagonist (e.g., an anti-TMPRSS2 antibody), a MSLN antagonist (e.g., an anti-MSLN antibody), a CA9 antagonist (e.g., an anti-CA9 antibody), a uroplakin antagonist (e.g., an anti-uroplakin antibody), etc.

Other agents that may be beneficially administered in combination with the protein-drug conjugates (e.g., antibody-drug conjugates), linker-payloads and payloads of the disclosure include cytokine inhibitors, including small-molecule cytokine inhibitors and antibodies that bind to cytokines such as IL-1, IL-2, IL-3, IL-4, IL-5, IL-6, IL-8, IL-9, IL-11, IL-12, IL-13, IL-17, IL-18, or to their respective receptors. The pharmaceutical compositions of the present disclosure (e.g., pharmaceutical compositions comprising an anti-HER2, an anti-HER2/HER2 bispecific, an anti-MET, an anti-MET/MET bispecific, or an anti-STEAP2 protein-drug conjugate (e.g., antibody-drug conjugate as disclosed herein) may also be administered as part of a therapeutic regimen comprising one or more therapeutic combinations selected from “ICE”: ifosfamide (e.g., Ifex®), carboplatin (e.g., Paraplatin®), etoposide (e.g., Etopophos®, Toposar®, VePesid®, VP-16); “DHAP”: dexamethasone (e.g., Decadron®), cytarabine (e.g., Cytosar-U®, cytosine arabinoside, ara-C), cisplatin (e.g., Platinol®-AQ); and “ESHAP”: etoposide (e.g., Etopophos®, Toposar®, VePesid®, VP-16), methylprednisolone (e.g., Medrol®), high-dose cytarabine, cisplatin (e.g., Platinol®-AQ).

The present disclosure also includes therapeutic combinations comprising any of the protein-drug conjugates (e.g., antibody-drug conjugates), linker-payloads and payloads mentioned herein and an inhibitor of one or more of HER2, VEGF, Ang2, DLL4, EGFR, ErbB2, ErbB3, ErbB4, EGFRvIII, cMet, IGF1R, B-raf, PDGFR-α, PDGFR-β, FOLH1 (PSMA), PRLR, STEAP1, STEAP2, TMPRSS2, MSLN, CA9, uroplakin, or any of the aforementioned cytokines, wherein the inhibitor is an aptamer, an antisense molecule, a ribozyme, an siRNA, a peptibody, a nanobody or an antibody fragment (e.g., Fab fragment; F(ab′)2 fragment; Fd fragment; Fv fragment; scFv; dAb fragment; or other engineered molecules, such as diabodies, triabodies, tetrabodies, minibodies and minimal recognition units). The antigen-binding molecules of the disclosure may also be administered and/or co-formulated in combination with antivirals, antibiotics, analgesics, corticosteroids and/or NSAIDs. The antigen-binding molecules of the disclosure may also be administered as part of a treatment regimen that also includes radiation treatment and/or conventional chemotherapy.

The additional therapeutically active component(s) may be administered just prior to, concurrent with, or shortly after the administration of an antigen-binding molecule of the present disclosure; (for purposes of the present disclosure, such administration regimens are considered the administration of an antigen-binding molecule “in combination with” an additional therapeutically active component).

The present disclosure includes pharmaceutical compositions in which protein-drug conjugates (e.g., antibody-drug conjugates), linker-payloads and/or payloads of the present disclosure are co-formulated with one or more of the additional therapeutically active component(s) as described elsewhere herein.

### Administration Regimens

According to certain embodiments of the present disclosure, multiple doses of a protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or a payload may be administered to a subject over a defined time course. The methods according to this aspect of the disclosure comprise sequentially administering to a subject multiple doses of a protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or a payload of the disclosure. As used herein, “sequentially administering” means that each dose of a protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or a payload is administered to the subject at a different point in time, e.g., on different days separated by a predetermined interval (e.g., hours, days, weeks or months). The present disclosure includes methods which comprise sequentially administering to the patient a single initial dose of a protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or a payload, followed by one or more secondary doses of the protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or payload, and optionally followed by one or more tertiary doses of the a protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or payload.

The terms “initial dose,” “secondary doses,” and “tertiary doses,” refer to the temporal sequence of administration of the protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or payload of the disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the protein-drug conjugate (e.g., an anti-HER2, or an anti-HER2/HER2 bispecific, an anti-MET, an anti-MET/MET bispecific, or an anti-STEAP2 antibody-drug conjugate), linker-payload and/or payload, but generally may differ from one another in terms of frequency of administration. In certain embodiments, however, the amount of the protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or payload contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment of the present disclosure, each secondary and/or tertiary dose is administered 1 to 26 (e.g., 1, 1%, 2, 2%, 3, 3%, 4, 4%, 5, 5%, 6, 6%, 7, 7%, 8, 8%, 9, 9%, 10, 10%, 11, 11%, 12, 12%, 13, 13%, 14, 14%, 15, 15%, 16, 16%, 17, 17%, 18, 18%, 19, 19%, 20, 20%, 21, 21%, 22, 22%, 23, 23%, 24, 24%, 25, 25%, 26, 26%, or more) weeks after the immediately preceding dose. The phrase “the immediately preceding dose,” as used herein, means, in a sequence of multiple administrations, the dose of a protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or payload which is administered to a patient prior to the administration of the very next dose in the sequence with no intervening doses.

The methods according to this aspect of the disclosure may comprise administering to a patient any number of secondary and/or tertiary doses of a protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or payload. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### Examples

The following examples illustrate specific aspects of the instant description. The examples should not be construed as limiting, as the examples merely provide specific understanding and practice of the embodiments and their various aspects.

### Abbreviations

| Abbreviation | Term |
|---|---|
| aq | aqueous |
| Conc. | concentrated/concentration |
| COT | cyclooctyne |
| CPT | Camptothecins |
| DBU | 1,8-diazabicyclo[5.4.0]undec-7-ene |
| DCC | dicyclohexylcarbodiimide |
| DCE | 1,2-dichloroethane |
| DCM | dichloromethane |
| DCU | N,N′-dicyclohexylurea |
| DIPEA | diisopropylethyl amine |
| DMF | N,N-dimethylformamide |
| DMSO | dimethylsulfoxide |
| DXd | deruxtecan |
| Fmoc | (9H-fluoren-9-yl)methyl carbono |
| g/kg | gram/kilogram |
| Gly | glycine |
| HATU | 2-(7-azabenzotriazol-1-yl)-N,N,N′,N′-tetramethyluronium |
| | hexafluorophosphate |
| HOAt | 1-hydroxy-7-azabenzotriazole |
| HOBt | 1-hydroxybenzotriazole |
| HOSu | N-hydroxysuccinimide |
| HPLC | high performance liquid chromatography |
| HYP | 2-hydroxypyridine |
| L | liters |
| LC | liquid chromatography |
| M/mM | molar/millimolar |
| MeHYQ | 4-methyl-2-hydroxyquinoline |
| MS | mass spectrometry |
| MTBE | methyl tert-butyl ether |
| NMR | nuclear magnetic resonance |
| Oxyma Pure | ethyl cyanoglyoxylate-2-oxime |
| PEG | polyethylene glycol |
| PMA | phosphomolybdic acid hydrate |
| PNP | bis(4-nitrophenyl) carbonate |
| ppm | parts per million (chemical shift) |
| PPTS | pyridium p-toluenesulfonate |
| RP | reversed phase |
| Sat. | saturated |
| TFA | trifluoroacetic acid |
| THF | tetrahydrofuran |
| TLC | thin layer chromatography |
| Top | topoisomerase |
| vcPAB | Val-Cit-para-aminobenzylalcohol |
| wt | weight |

### General Methods

### Example 1. Synthesis of Payloads

Two synthetic routes were designed to make prodrugs of DXd shown in Scheme 1.
Route A was using Exatecan reacting with 4, see Scheme 2A.
Route B was using DXd reacting with 3, see Scheme 2B.

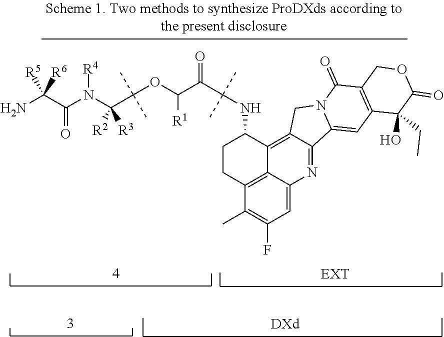

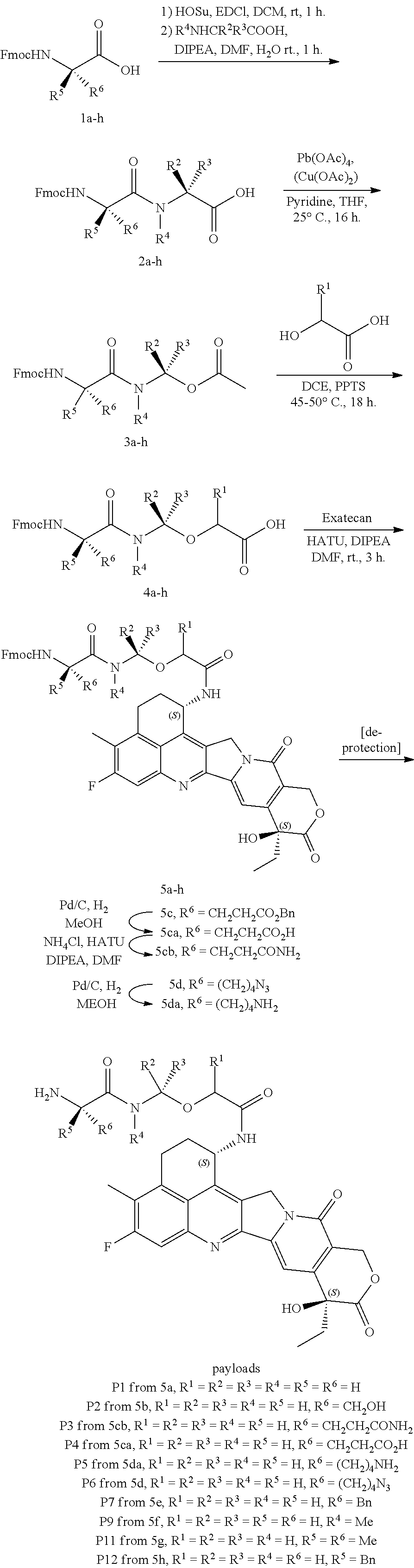

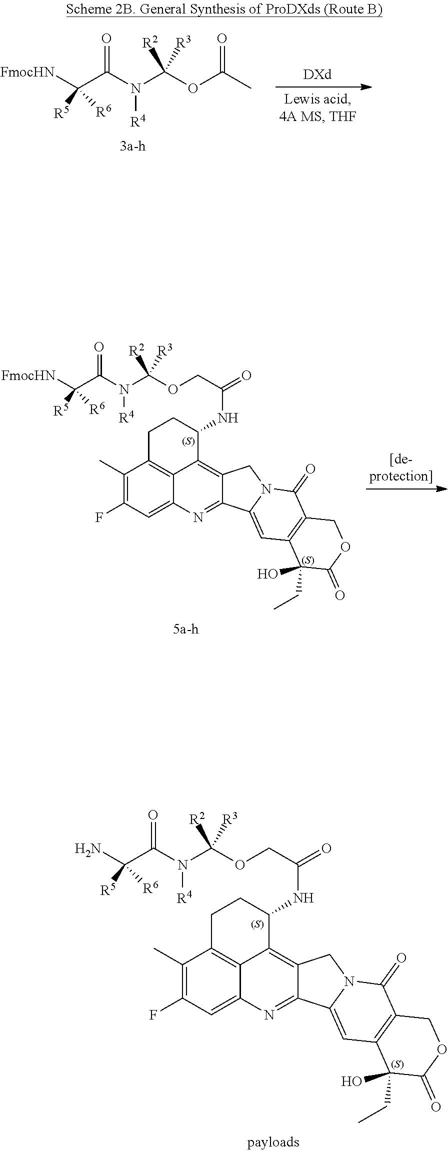

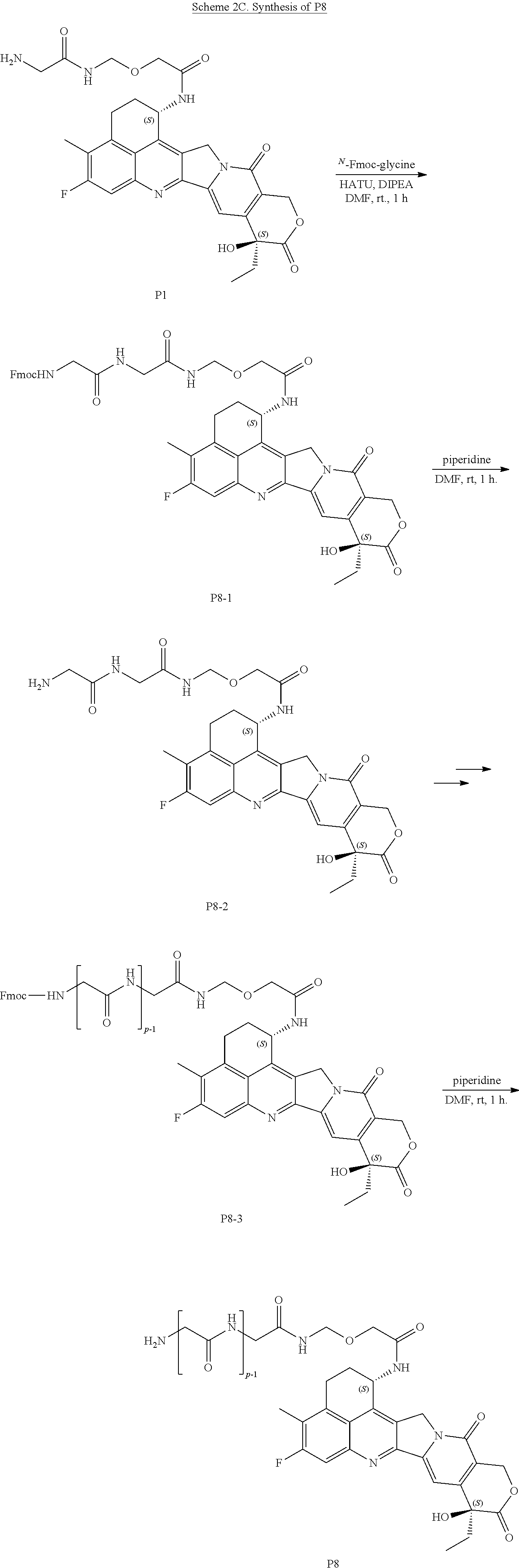

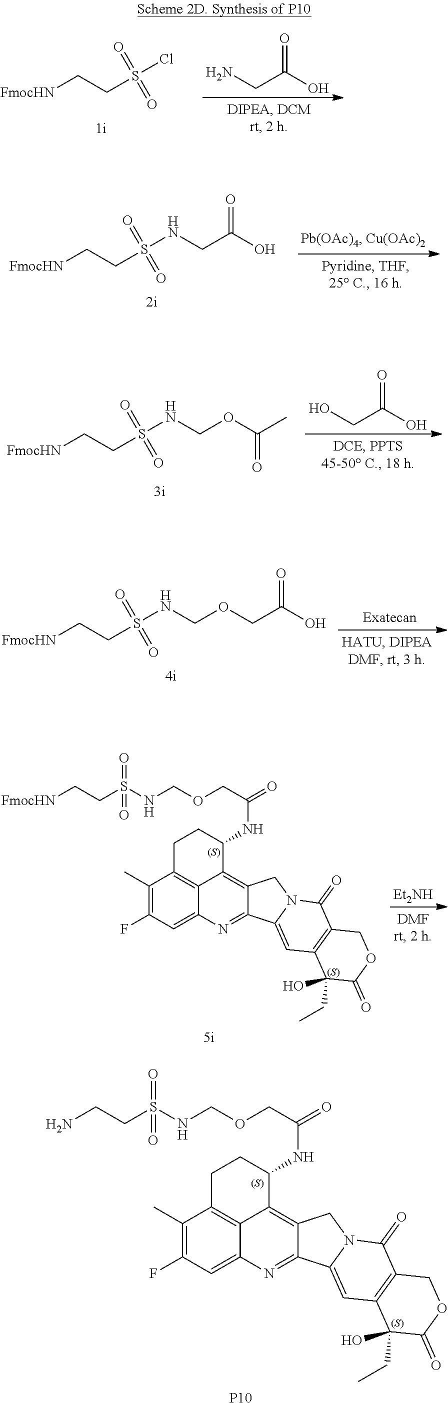

### Example 2. Synthesis of Linker-Payloads

Five synthetic routes are summarized in Scheme 3, below, based on the last step in the route. All building blocks (A to F) have suitable reactive moieties to be used in the reactions. The synthetic schemes of the building blocks and the final linker-payloads are illustrated as follows.
Route 1 was using fragment F with Exatecan.
Route 2 was using fragment E with DXd.
Route 3 was using fragment D with Prodrug or Fmoc protected ProDrug.
Route 4 was using fragment B with vcPABC-ProDrug
Route 5 was using fragment A with PEG4-vcPABC-ProDrug

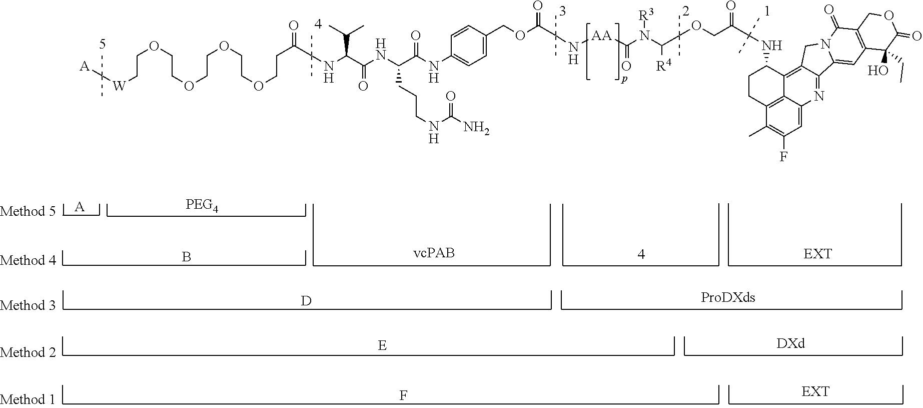

Intermediates A and B can be purchased commercially or reported building blocks which have functional groups that can be conjugated with antibody by, e.g., a bio-orthogonal (“click”) reaction (Table A).

TABLE A
Linker-payload with generic reactive moiety A

| | | | | | | Inter- | |
|---|---|---|---|---|---|---|---|
| | | | | | | mediate B | |
| | | | | | | Y-W-PEGn- | |
| | | | | | | COOH or its | |
| | | Group | Intermediate A | | | active ester | |
| Abbr. | Group A | W | # | Structure of A | CAS# | # | CAS# |
| COT | 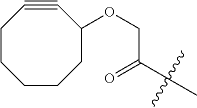 [SMILES: *C(=O)COC1C#CCCCCC1] | NH | Aa | 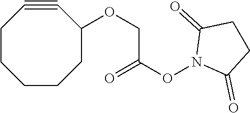 [SMILES: O=C(COC1C#CCCCCC1)ON1C(=O)CCC1=O] | 1425803- 45-7 | Ba | 1802907- 91-0 |
| DIBAC | 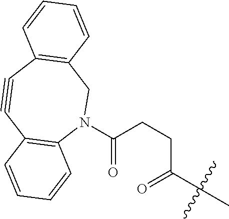 [SMILES: *C(=O)CCC(=O)N1Cc2ccccc2C#Cc2ccccc21] | NH | Ae | 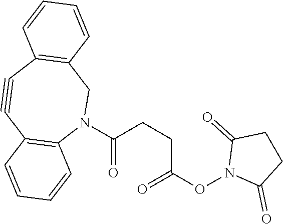 [SMILES: O=C(CCC(=O)N1Cc2ccccc2C#Cc2ccccc21)ON1C(=O)CCC1=O] | 1353016- 71-3 | Be | 1537170- 85-6 |
| DIBAC- NH | 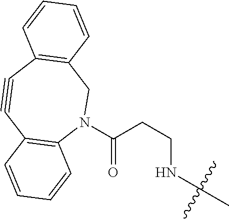 [SMILES: *NCCC(=O)N1Cc2ccccc2C#Cc2ccccc21] | CO | Ak | 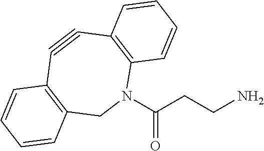 [SMILES: NCCC(=O)N1Cc2ccccc2C#Cc2ccccc21] | 1255942- 06-3 | Bk | 2110448- 99-0 |
| Mc | 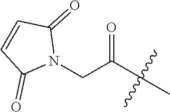 [SMILES: *C(=O)CN1C(=O)C=CC1=O] | NH | Af | 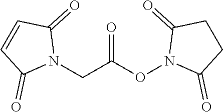 [SMILES: O=C(CN1C(=O)C=CC1=O)ON1C(=O)CCC1=O] | 55750- 61-3 | Bf | 1954756- 73-0 |
| BCN | 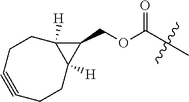 [SMILES: *C(=O)OC[C@@H]1[C@@H]2CCC#CCC[C@@H]21] | NH | Ag | 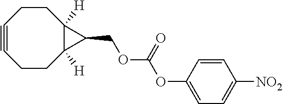 [SMILES: O=C(OC[C@@H]1[C@@H]2CCC#CCC[C@@H]21)Oc1ccc([N+](=O)[O-])cc1] | 1263166- 91-1 | Bg | 1881221- 47-1 |
| Norbor- neneol | 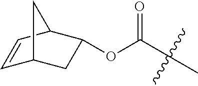 [SMILES: *C(=O)OC1CC2C=CC1C2] | NH | Ah | 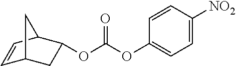 [SMILES: O=C(Oc1ccc([N+](=O)[O-])cc1)OC1CC2C=CC1C2] | 2206682- 09-7 | | |
| Cyclo- propene- OH | 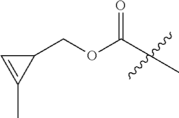 [SMILES: *C(=O)OCC1C=C1C] | NH | Ai | 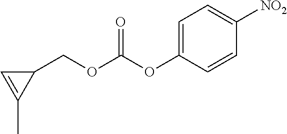 [SMILES: CC1=CC1COC(=O)Oc1ccc([N+](=O)[O-])cc1] | 1515861- 58-1 | | |
| Cyclo- propene- COOH | 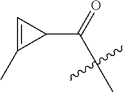 [SMILES: *C(=O)C1C=C1C] | NH | Aj | 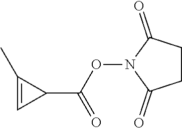 | 39492- 17-6 | | |
| MeTz- Ph | 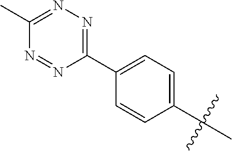 [SMILES: *c1ccc(-c2nnc(C)nn2)cc1] | O | Ab | 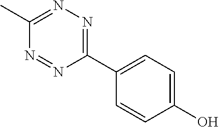 | 58884- 35-8 | Bb | 1802907- 91-0 |
| TCO | 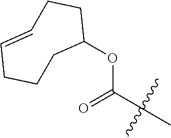 [SMILES: *C(=O)OC1CC/C=C/CCC1] | NH | Ac | 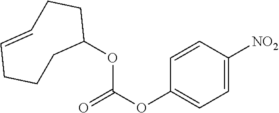 [SMILES: O=C(Oc1ccc([N+](=O)[O-])cc1)OC1CC/C=C/CCC1] | 1380416- 34-1 | Bc | 1897439- 90-5 |
| MeTz- PhAc | 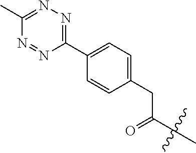 [SMILES: *C(=O)Cc1ccc(-c2nnc(C)nn2)cc1] | NH | Ad | 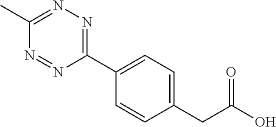 [SMILES: Cc1nnc(-c2ccc(CC(=O)O)cc2)nn1] | 1380500- 88-8 | Bd | NA |
| MeTz- Bn-NH | 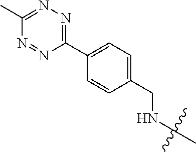 [SMILES: *NCc1ccc(-c2nnc(C)nn2)cc1] | CO | Al | 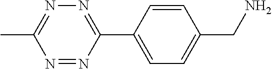 | 1345955- 28-3 | | |
| Tz-Bn- NH | 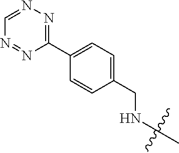 [SMILES: *NCc1ccc(-c2nncnn2)cc1] | CO | Am | 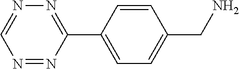 [SMILES: NCc1ccc(-c2nncnn2)cc1] | 1092689- 33-2 | | |

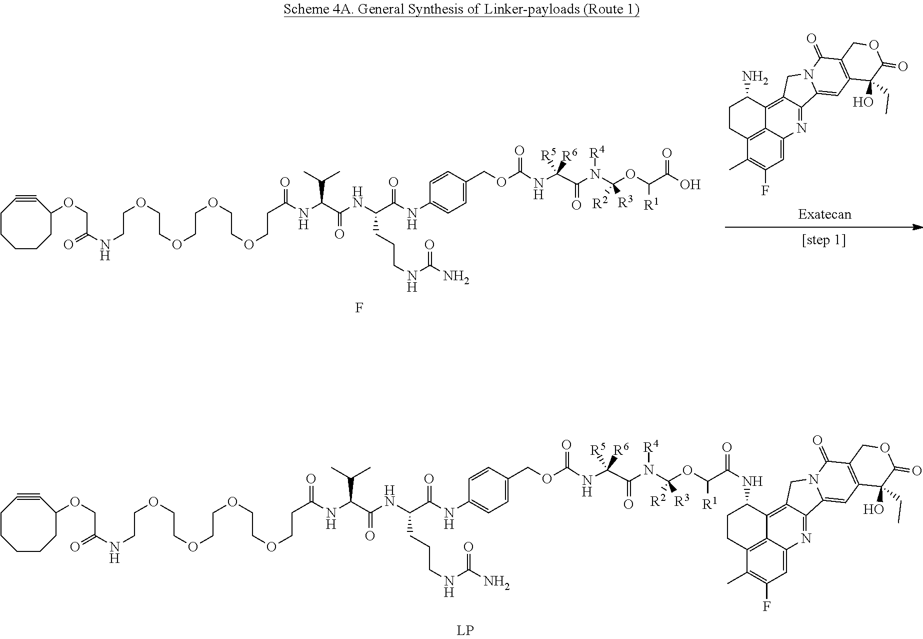

[condition] Exatecan, HATU, DIPEA, DMF, 25° C., 16 hours.

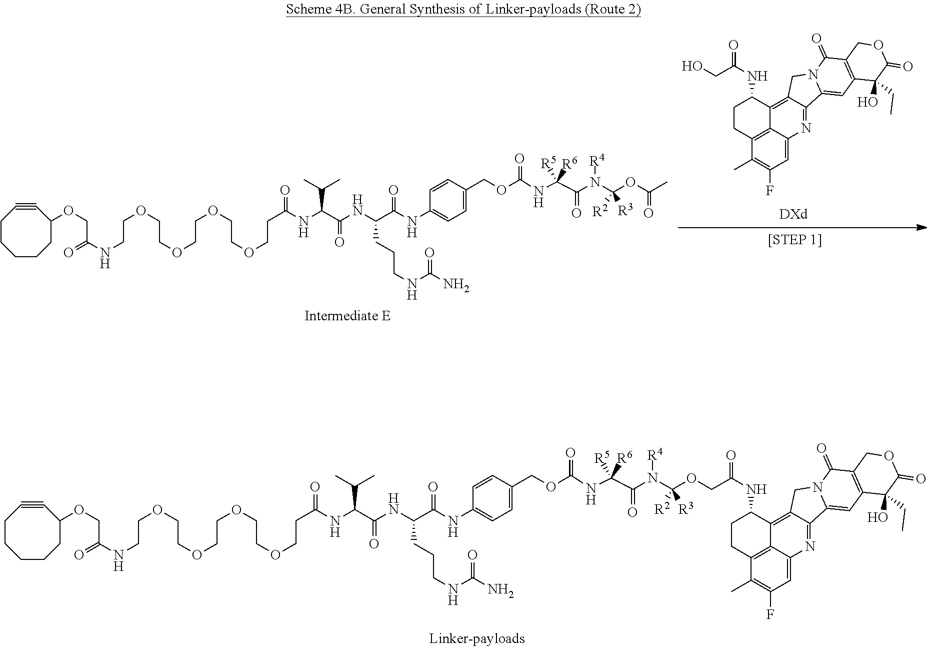

[condition] DXd, Tf₂NH, 4A MS, THF, 20° C., 10 min.

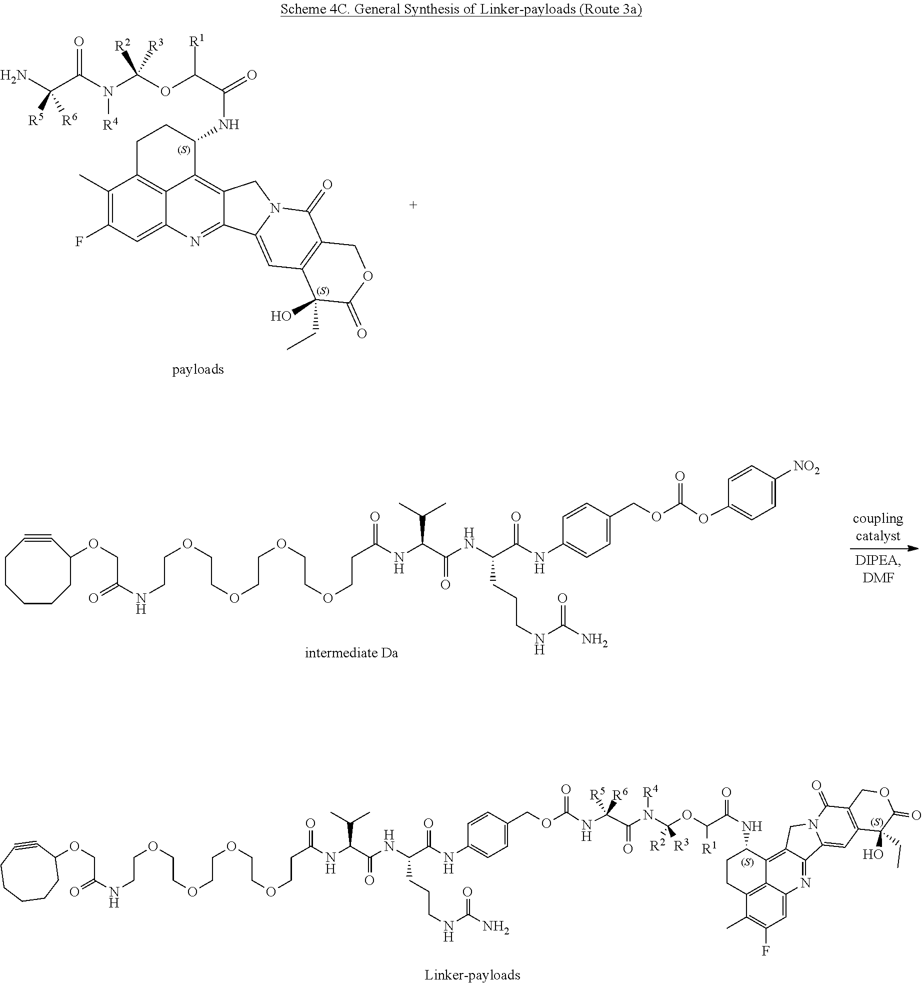

[condition] intermediate D, coupling catalyst 4-Hydroxy-2-methylquinoline (MeHYQ), DIPEA, DMF, rt., 2 hours

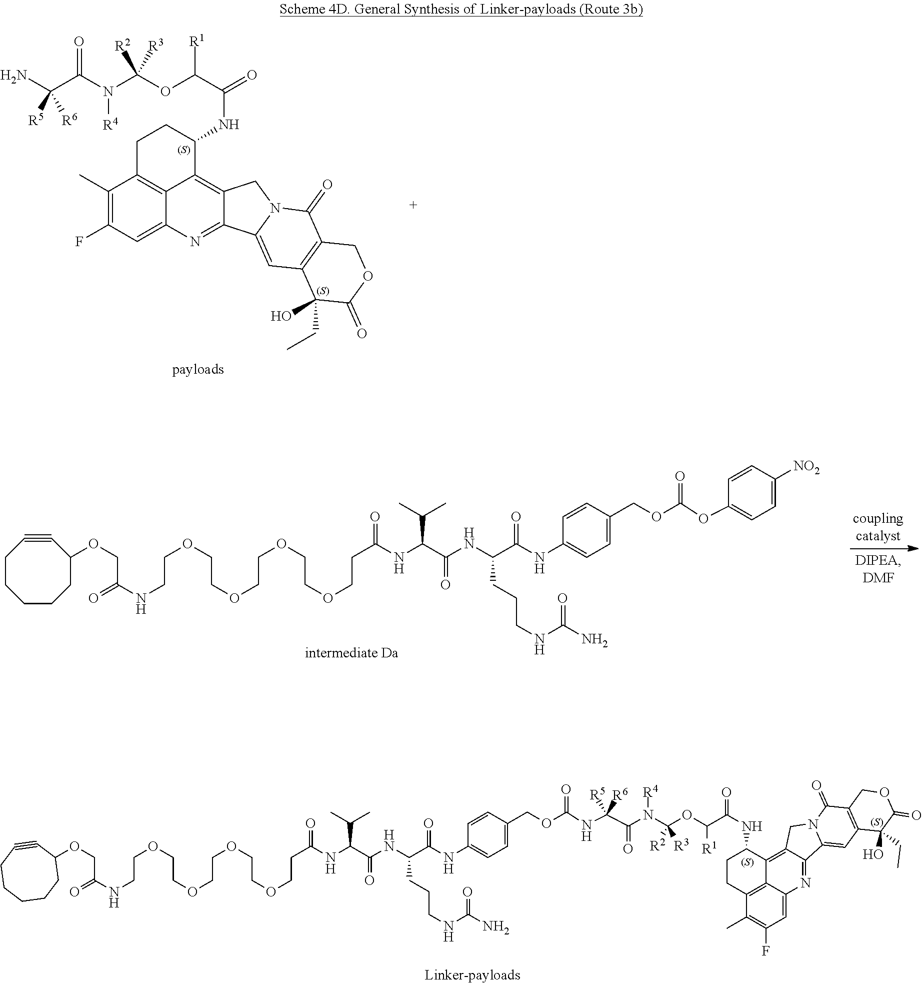

[condition] intermediate D, coupling catalyst 4-Hydroxy-2-methylquinoline (MeHYQ), Et₃N, DBU, DMF, 50° C., 6 hours.

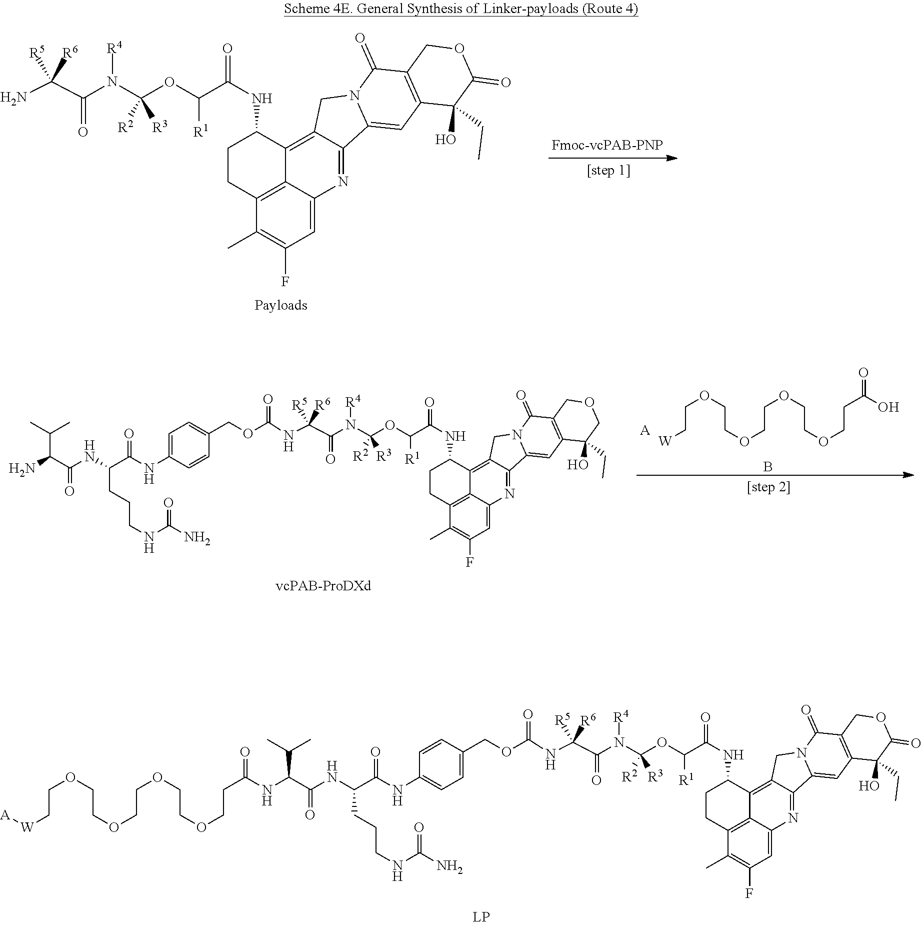

[condition] step 1, a) Fmoc-vcPAB-PNP, coupling reagent 4-Hydroxy-2-methylquinoline (MeHYQ), DIPEA, DMF, rt., 4 h.; b) Et₂NH, DMF, rt., 2 h. step2, intermediate B, HATU, DIPEA, DMF, rt., 4 h.

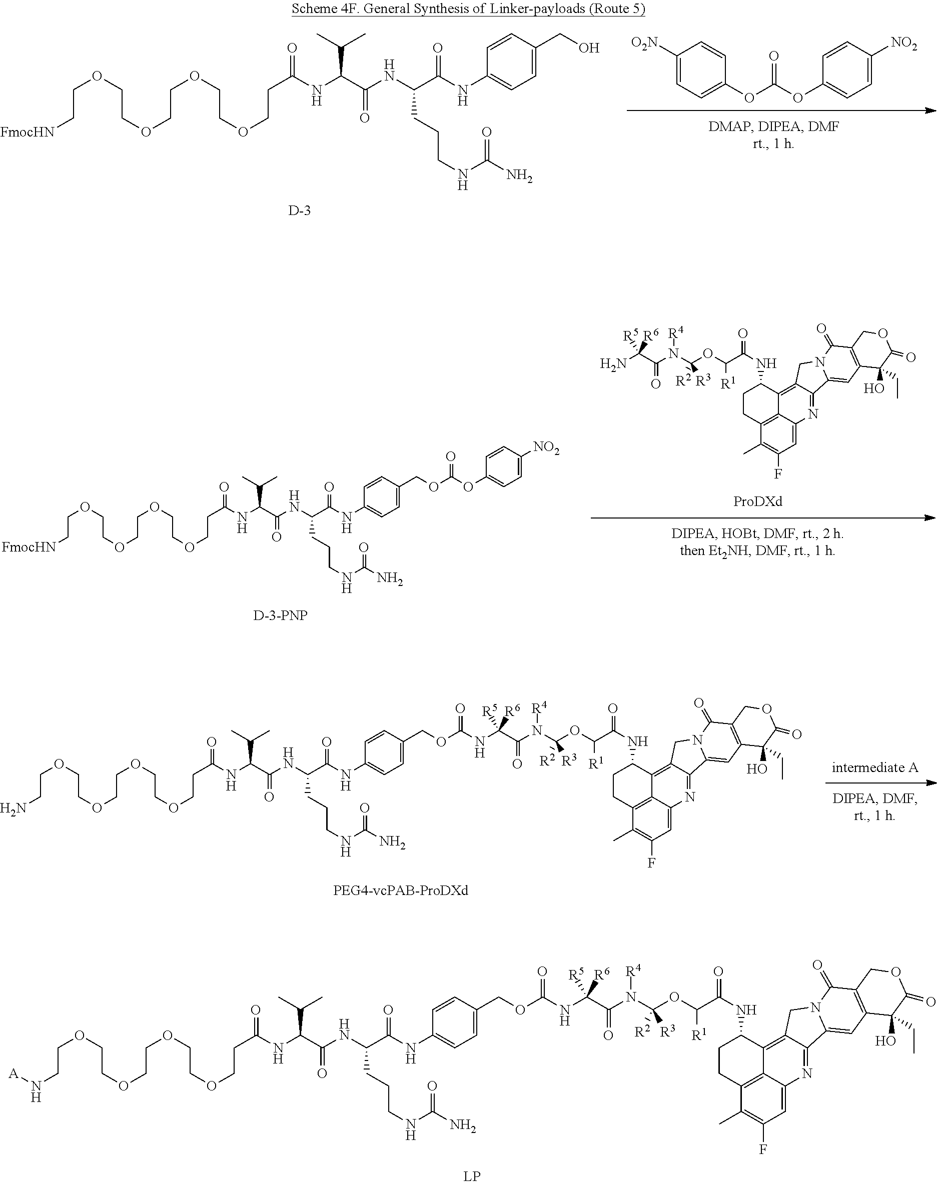

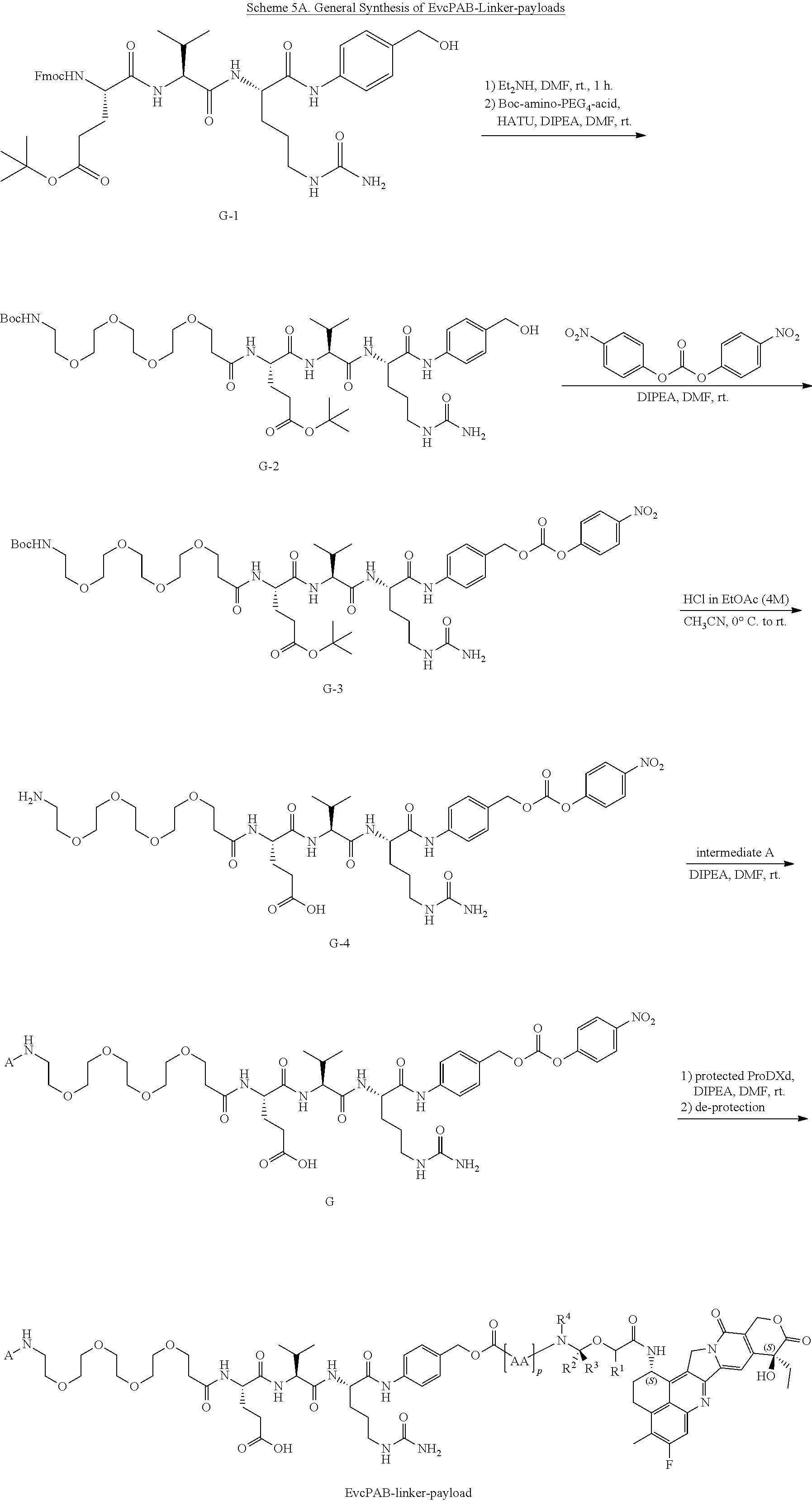

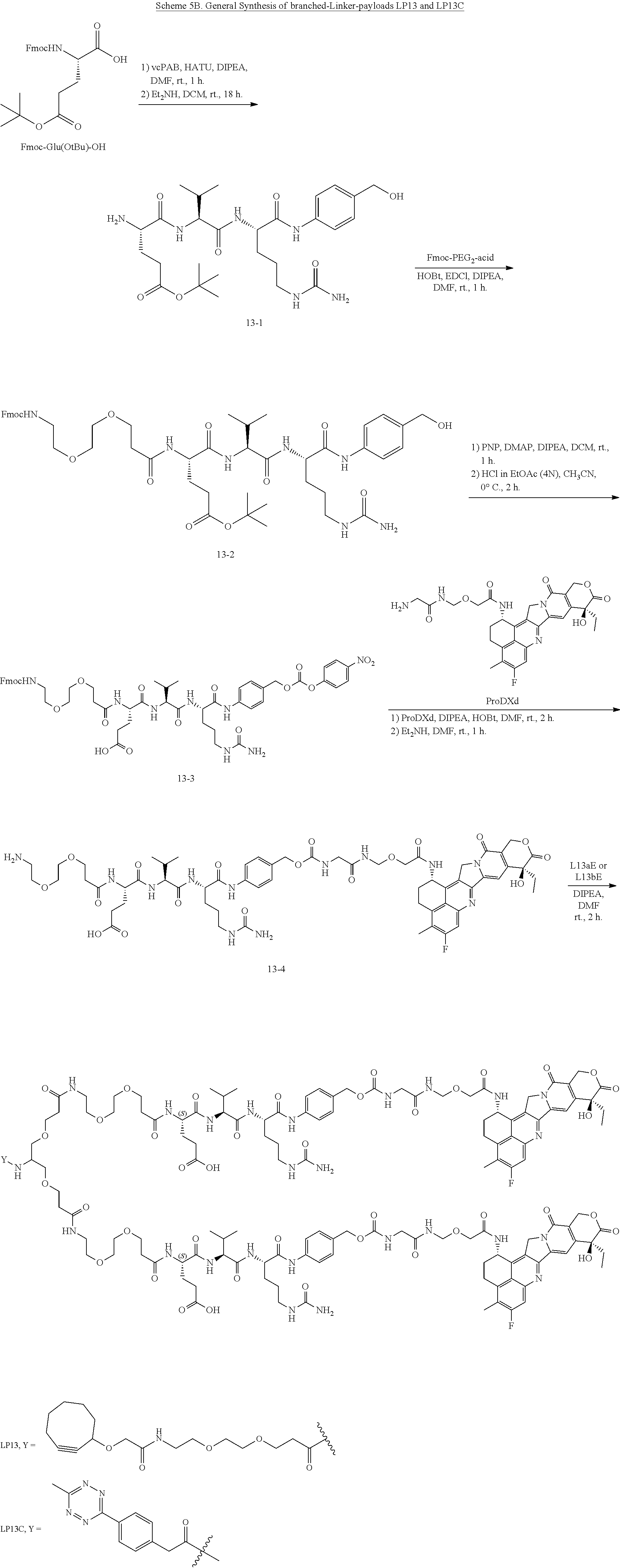

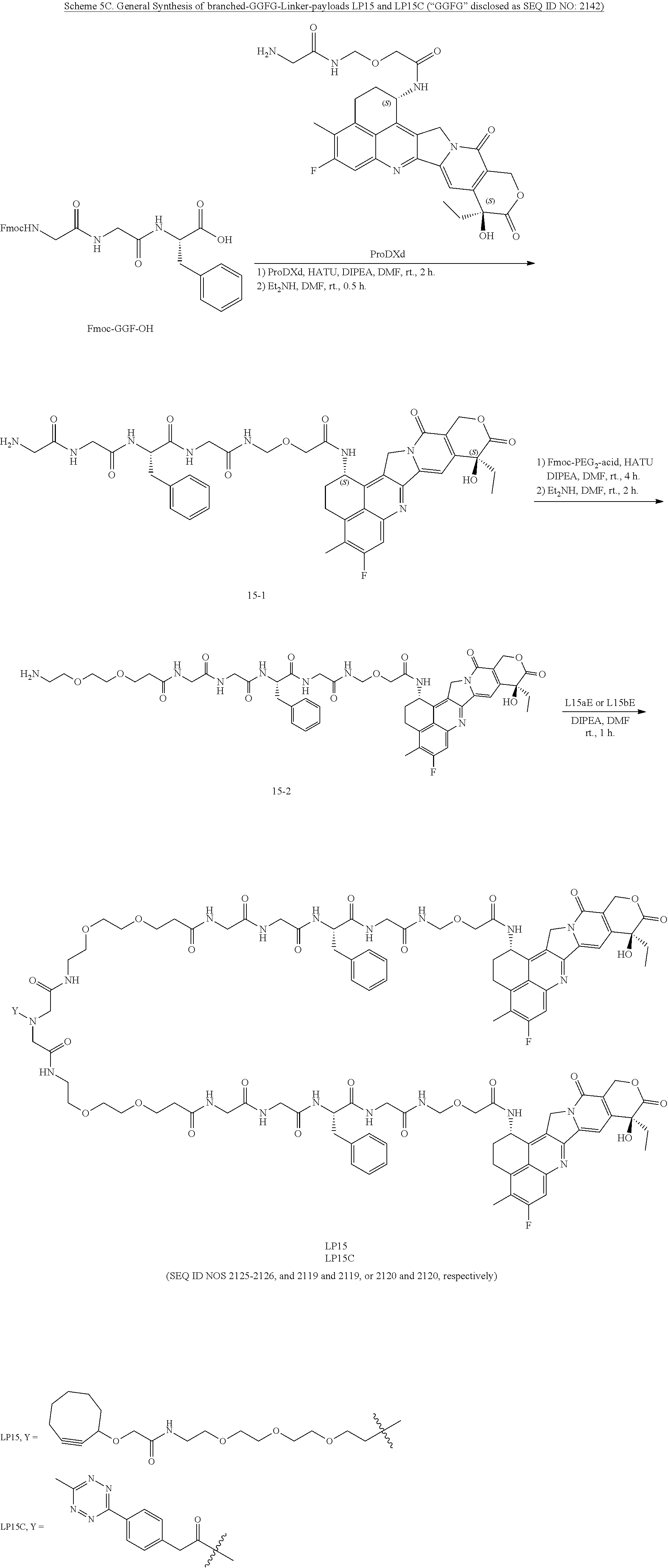

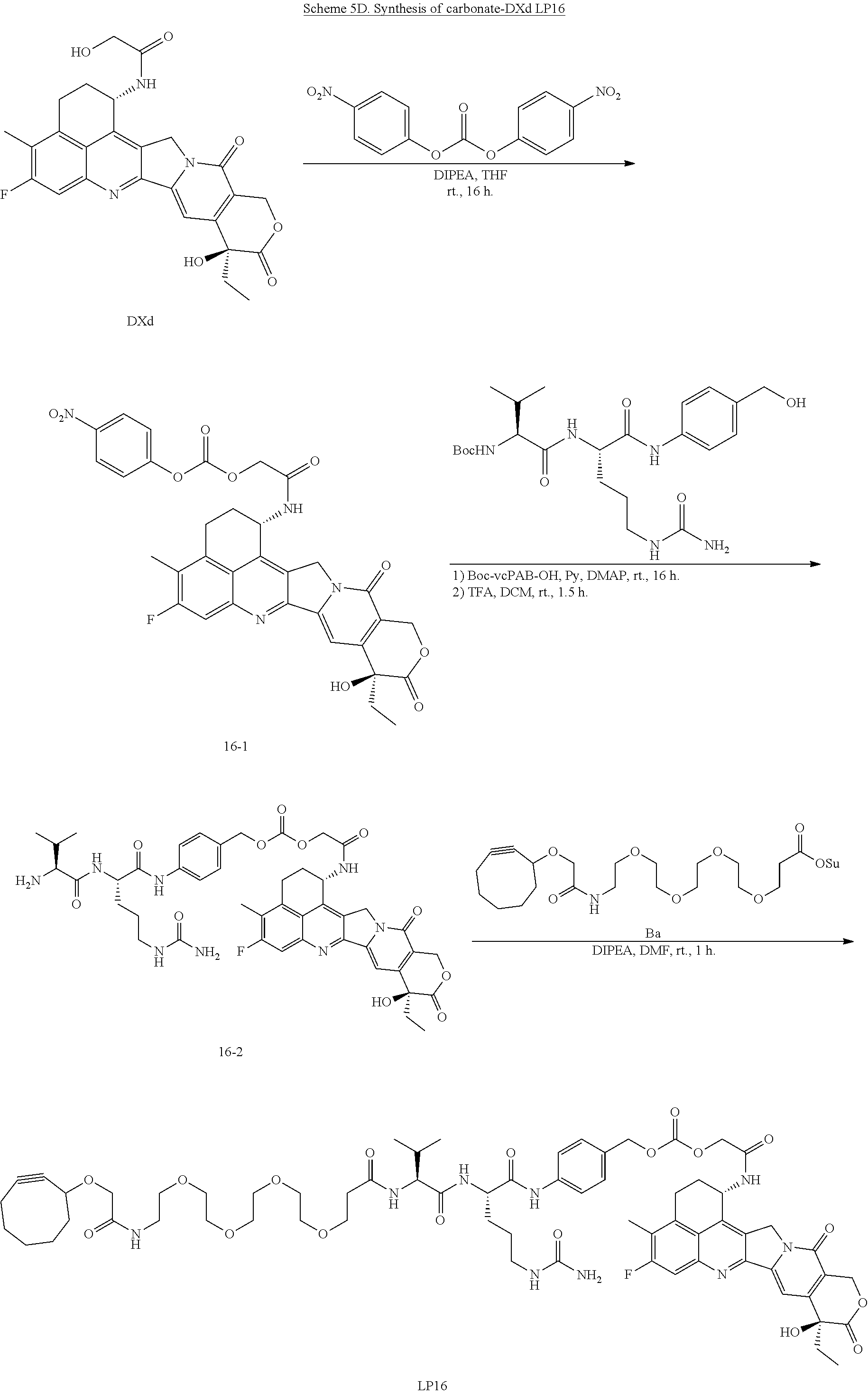

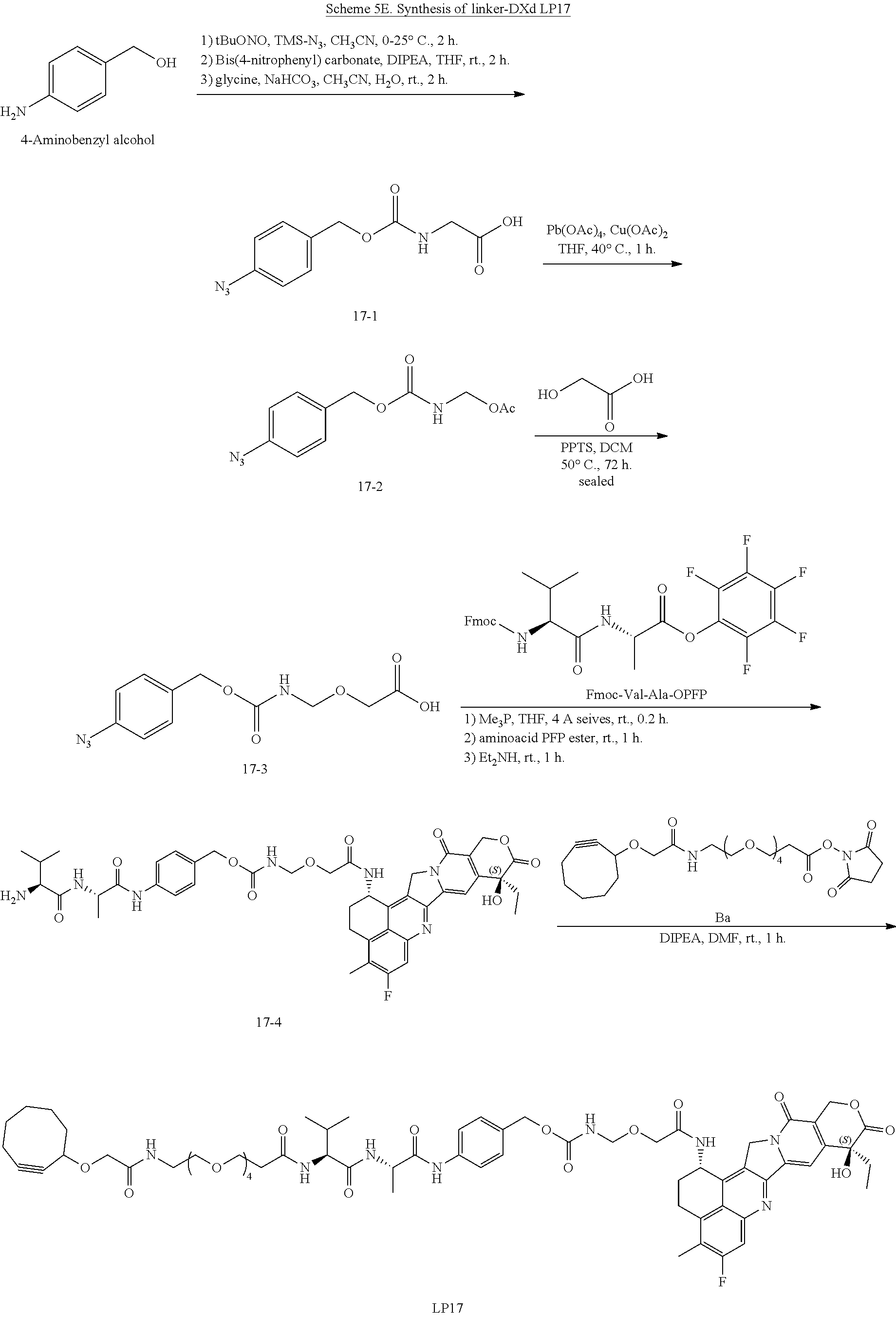

### Example 3. Synthesis of Key Intermediates/Building Blocks

Intermediate A was prepared according to Scheme 6 and the below descriptions.

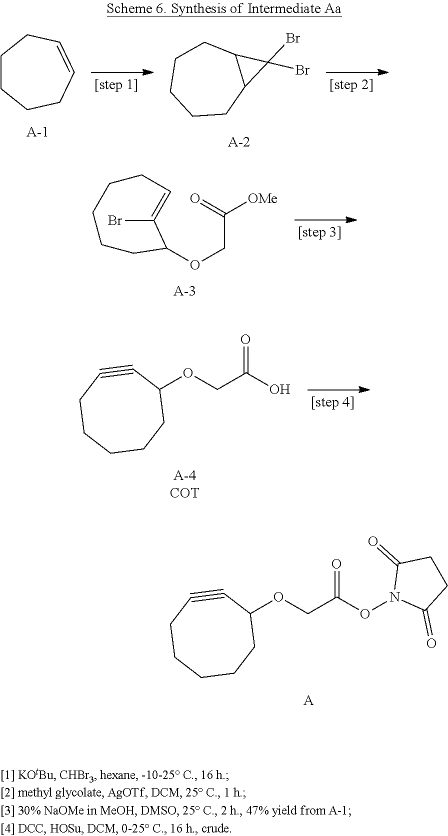

The synthesis of intermediate A-4 (COT) was reported in WO2010106245, and the synthesis of intermediate A was reported in WO2015143092, both of which are incorporated by reference herein in their entirety. (Scheme 6)

Intermediate B was prepared according to Scheme 7 and the below descriptions.

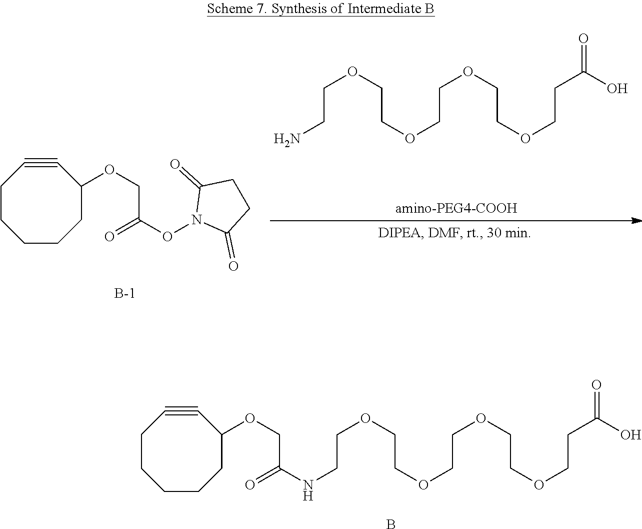

The synthesis of intermediate B was reported in WO2019094395 and described in Scheme 7, above.

The synthesis of intermediate 4a-h is described in Scheme 2A, above. Intermediate 4a is reported in WO2015146132 (Scheme 8). Alternatively, intermediate 4a was prepared by a 2-step synthesis with a 45% overall yield without chromatographic purification.

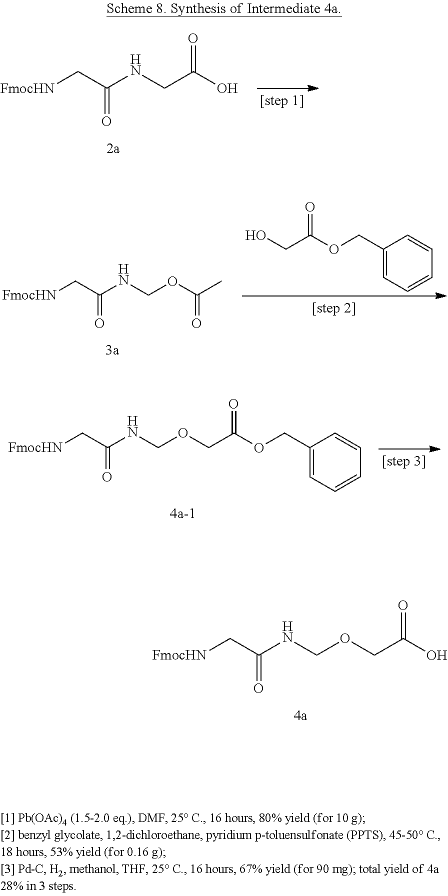

Alternatively, 4a was prepared at a larger scale by the following two-step procedure*:

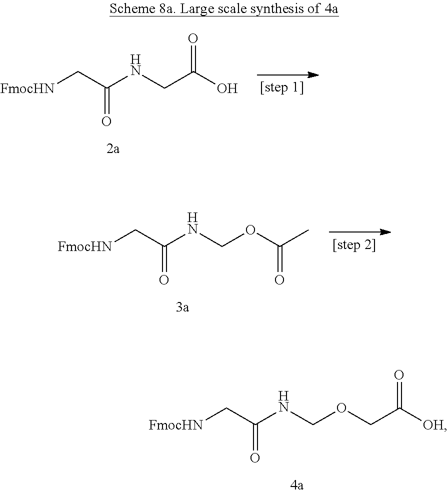

*wherein [Step 1] Cu(OAc)₂ (0.30 eq.), Pb(OAc)₄ (1.5-2.0 eq.), pyridine (2.0 eq.), THF, 25° C., 16 hours, 60% yield (for 0.80 kg); [Step 2] glycolic acid, 1,2-dichloroethane, pyridium p-toluenesulfonate (PPTS), 45-50° C., 18 hours, 75% yield (for 0.96 kg).

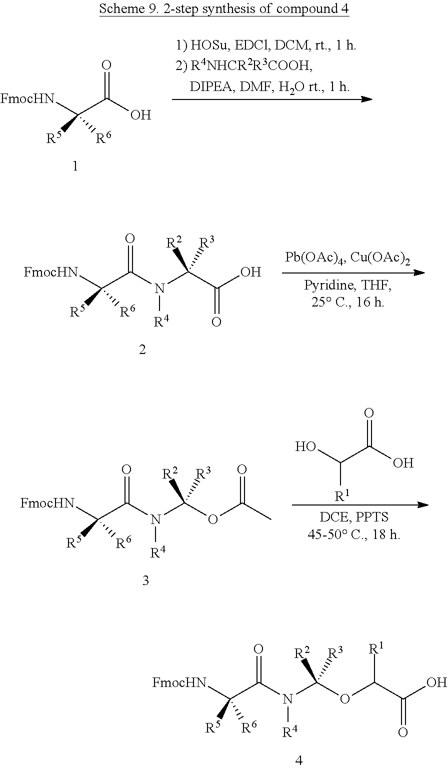

[condition] step 1, Cu(OAc)₂ (0.30 eq.), Pb(OAc)₄ (1.5-2.0 eq.), pyridine (2.0 eq.), THF, 25° C., 16 hours; step 2, glycolic acid, 1,2-dichloroethane, pyridium p-toluenesulfonate (PPTS), 45-50° C., 18 hours.

Two synthetic routes were summarized to make intermediate D in Scheme 10. All building blocks have suitable reactive moieties to be used in the reactions. The synthetic schemes of the building blocks and the final intermediate D are illustrated as follows.

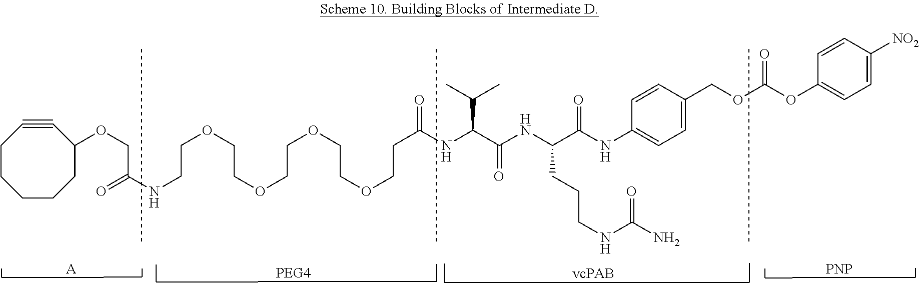

Route Da was from A to A-PEG4 (B), to A-PEG4-vcPAB, to A-PEG4-vcPAB-PNP (D).
Route db was from A with A-PEG4-vcPAB (B), then to A-PEG4-vcPAB-PNP (D)

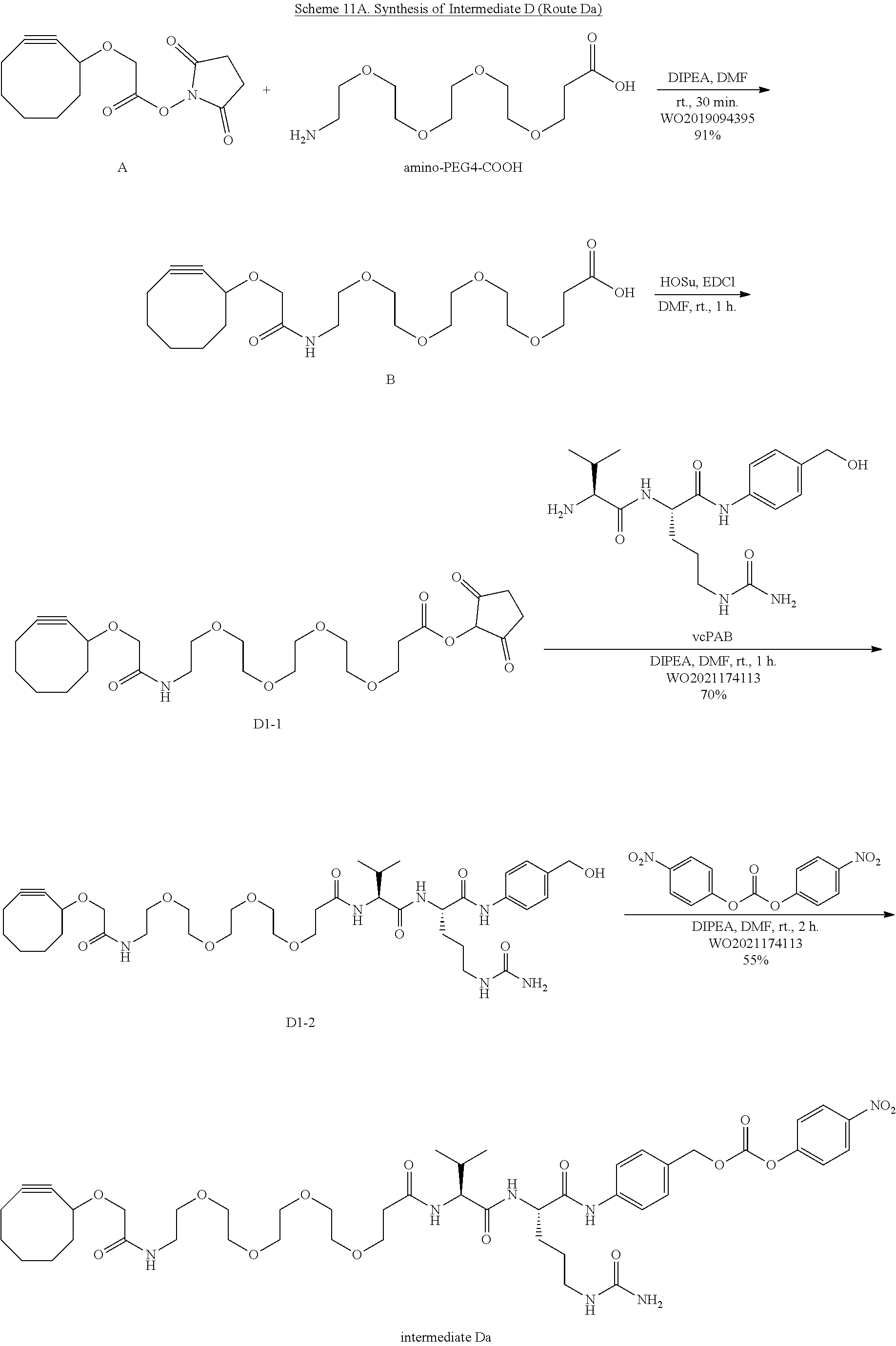

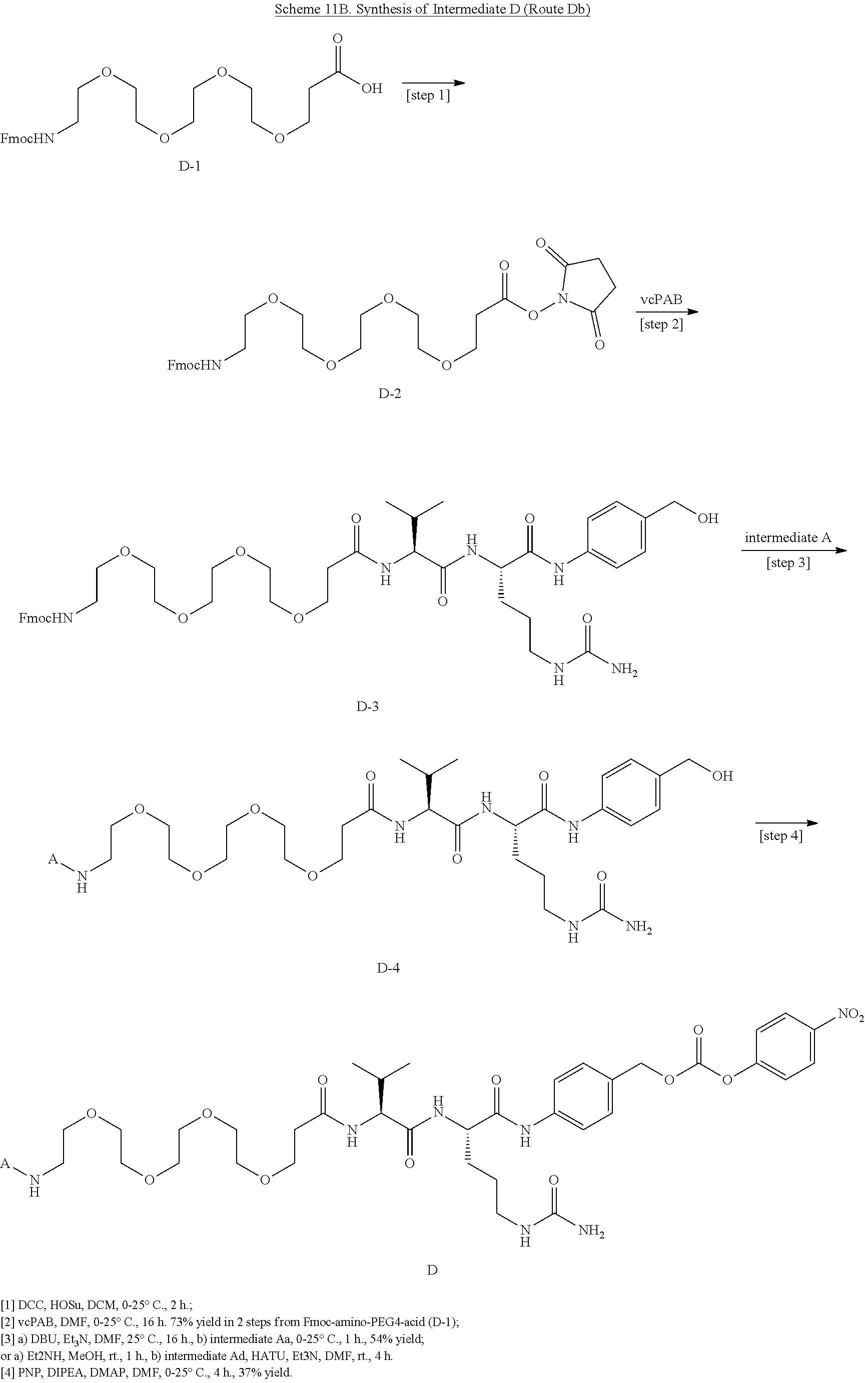

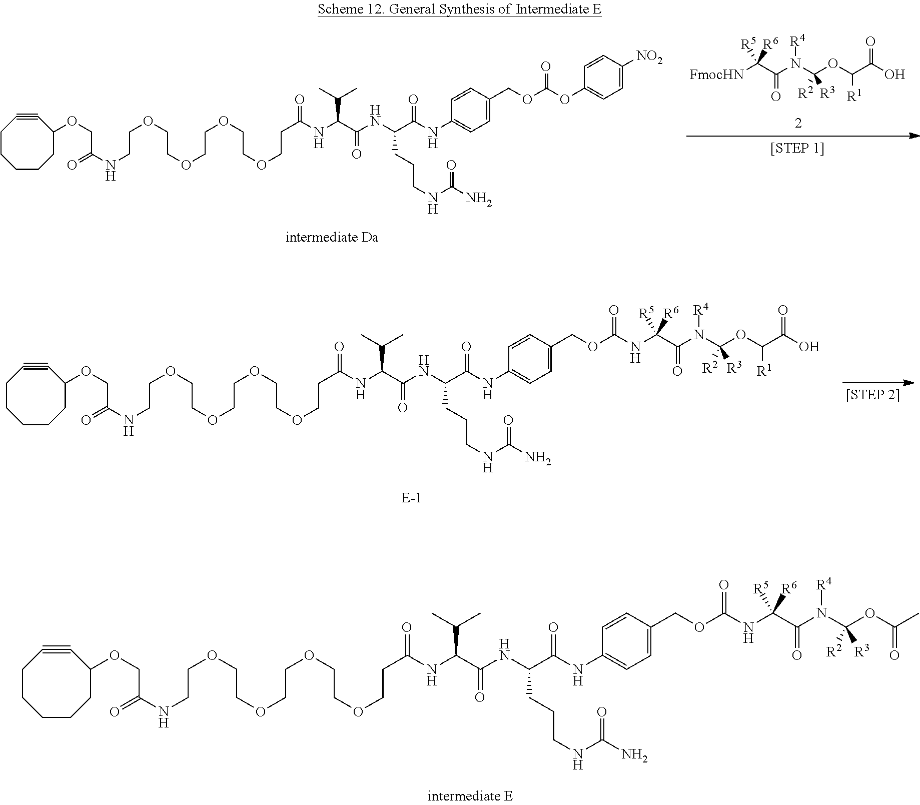

[step 1] a) compound 2, DBU, Et₃N, DMF, 25° C., 16 hr.; b) intermediate D, HOAt, DIPEA, 25° C., 4 hr.;
[step 2] Pb(OAc), HOAc, DMF, 25° C., 16 hr

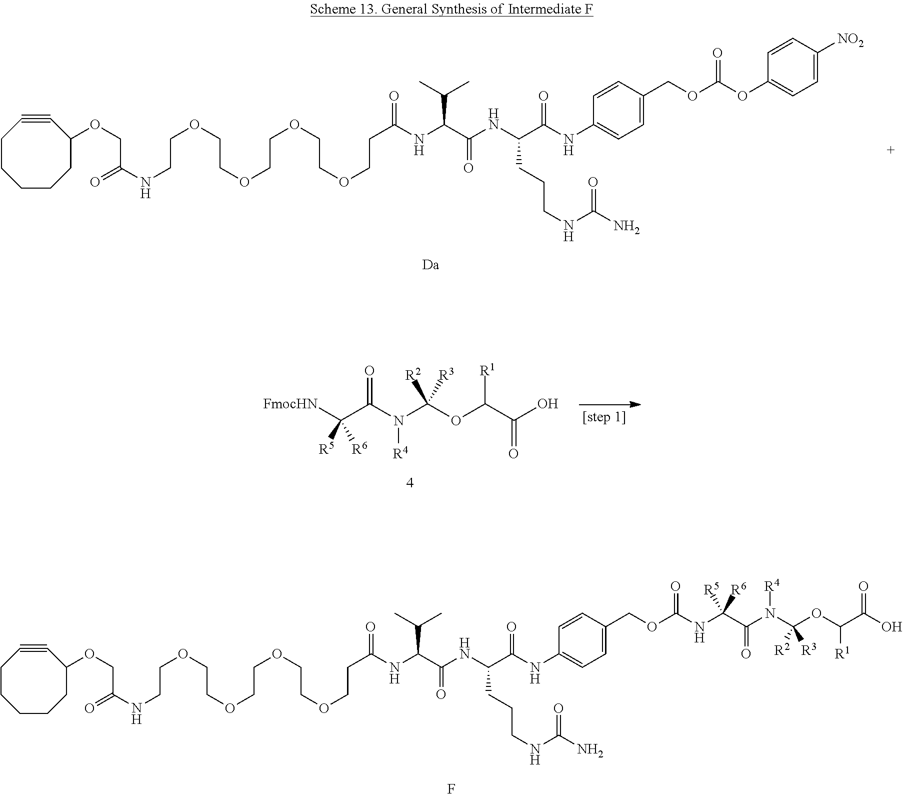

[STEP 1] a) intermediate 4, triethylamine, DBU, DMF, 25° C., 16 h, b) HOAt, intermediate D, 25° C., 16 hours.

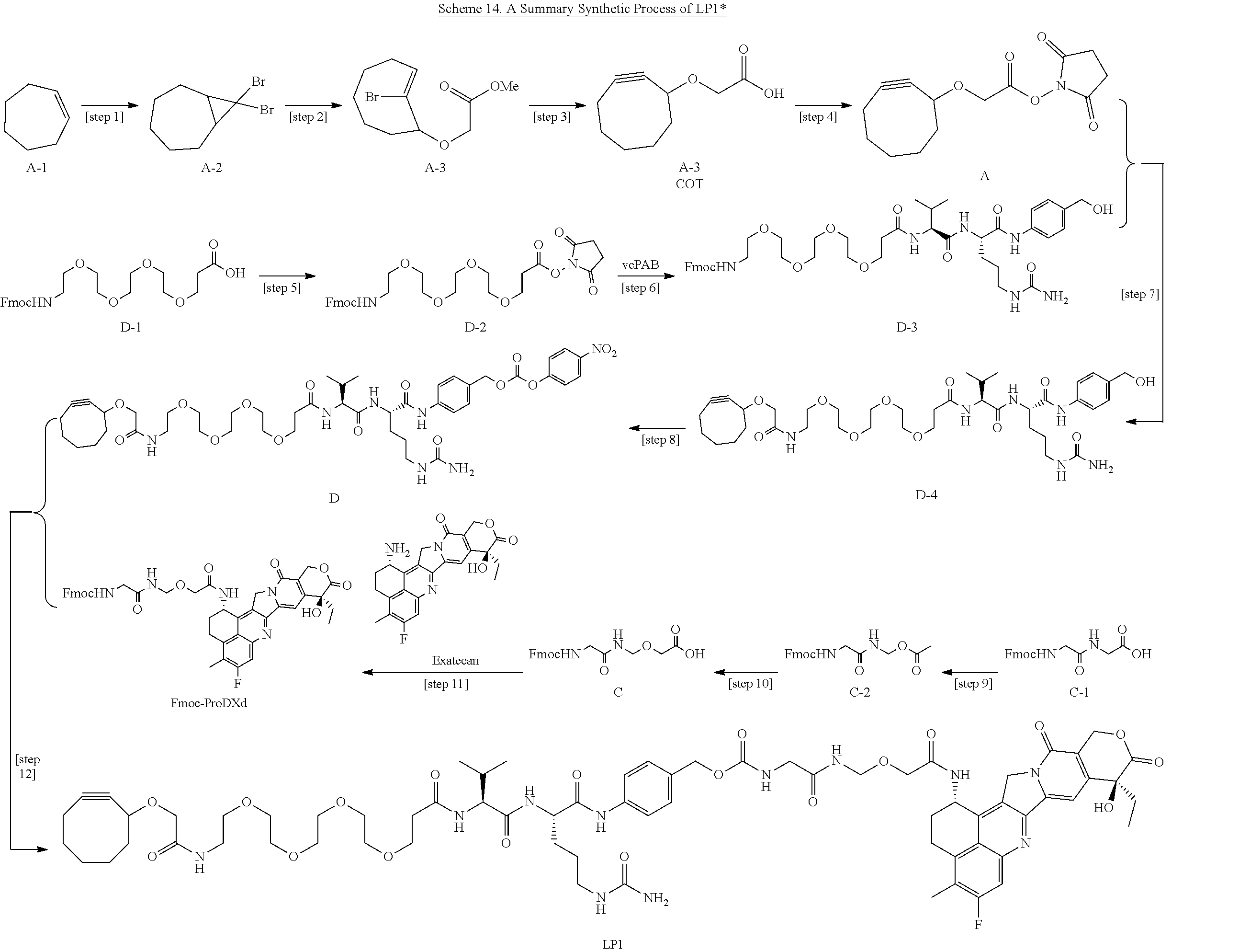

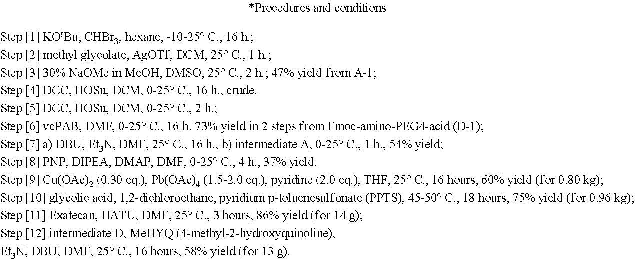

### Example 4. Conjugations

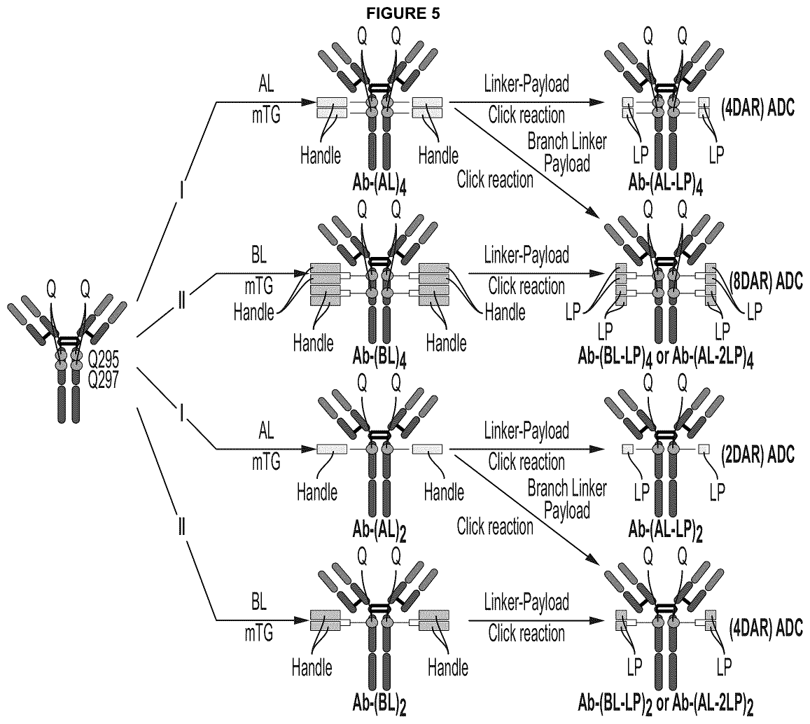

Site-specific ADCs conjugation is shown in FIG. 5.

Step 1 is site-specific conjugation of Handle-functionalized amine with an Antibody generated a drug conjugate containing 2, 4 or 8 handles per antibody. Here, AL=non-branched Handle-functionalized amine, BL=branched Handle-functionalized amine.

Step 2 is a click reaction between Handle-functionalized antibodies and a Linker-Payload (LP) to generate the site-specific ADCs.

### Synthesis of Payloads

### Example 5. General Synthesis of ProDXds (Scheme 2A)

General procedures of synthesis of compounds 2s

To a stirred solution of Fmoc protected amino-acid 1 (1 equiv.) in DCM (0.2 M) were added HOSu (2.2 equiv.) and EDCI (2.2 equiv.), and the reaction mixture was stirred at room temperature for 2 hours to 16 hours, which was monitored by LCMS. The mixture was diluted with DCM, washed with water (3×) and brine, dried over anhydrous sodium sulfate and concentrated in vacuo. The residue was dissolved in DMF (0.2 M). To the solution were added corresponding amino-acid (R³NHCHR⁴COOH) (1.0 equiv.) and DIPEA (3.0 equiv.), and the reaction mixture was stirred at room temperature for an hour, which was monitored by LCMS. The volatiles were removed in vacuo and the residue solution was directly purified by reversed phase flash chromatography (0-100% acetonitrile in aq. TFA (0.03%)) to give compound 2 (37-70% yield) as a white solid.

### 2-[2-({[(9H-fluoren-9-yl)methoxy]carbonyl}amino)acetamido]acetic acid (2a)

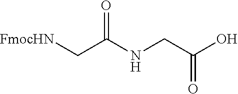

Commercially available.

### 2-[(2S)-3-[(tert-butyldimethylsilyl)oxy]-2-({[(9H-fluoren-9-yl)methoxy]carbonyl}amino)propanamido]acetic acid (2b)

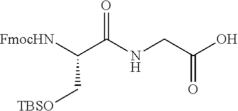

Following the general procedure, compound 2b (0.50 g, 54% yield) was obtained as a white solid. ESI m/z: 499 (M+H)⁺.

### 2-[(2S)-5-(benzyloxy)-2-({[(9H-fluoren-9-yl)methoxy]carbonyl}amino)-5-oxopentanamido]acetic acid (2c)

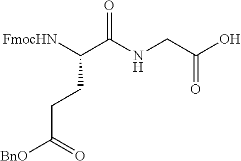

Following the general procedure, compound 2c (0.65 g, 58% yield) was obtained as a white solid. ESI m/z: 517 (M+H)⁺.

### 2-[(2S)-6-azido-2-({[(9H-fluoren-9-yl)methoxy]carbonyl}amino)hexanamido]acetic acid (2d)

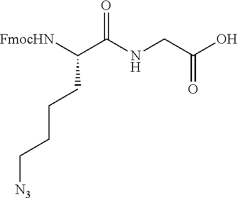

Following the general procedure, compound 2d (0.66 g, 70% yield) was obtained as a white solid. ESI m/z: 452 (M+H)⁺.

### 2-[(2S)-2-({[(9H-fluoren-9-yl)methoxy]carbonyl}amino)-3-phenylpropanamido]acetic acid (2e)

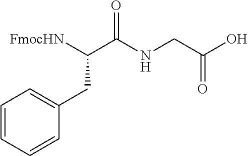

Following the general procedure, compound 2e (0.43 g, 37% yield) was obtained as a white solid. ESI m/z: 445 (M+H)⁺.

### 2-[2-({[(9H-fluoren-9-yl)methoxy]carbonyl}amino)-N-methylacetamido]acetic acid (2f)

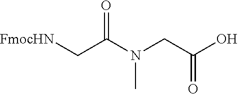

Following the general procedure, compound 2f (2.6 g, 72% yield) was obtained as a white solid. ESI m/z: 369 (M+H)⁺.

### 2-[2-({[(9H-fluoren-9-yl)methoxy]carbonyl}amino)-2-methylpropanamido]acetic acid (2g)

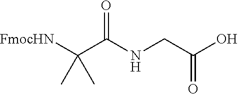

Following the general procedure, compound 2g (0.60 g, 50% yield) was obtained as a white solid. ESI m/z: 383 (M+H)⁺.

### 2-[(2R)-2-({[(9H-fluoren-9-yl)methoxy]carbonyl}amino)-3-phenylpropanamido]acetic acid (2h)

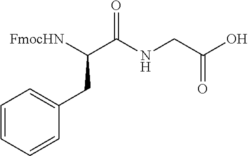

Following the general procedure, compound 2e (0.43 g, 37% yield) was obtained as a white solid. ESI m/z: 445 (M+H)⁺.

### General Procedures of Synthesis of Compounds 3a-h

To a 10 L reaction flask were added THF (0.25-0.30 M) and compound 2 (1.0 equiv.) at 25-30° C., and to the obtained suspension was added pyridine (2.0 equiv.) at 25-30° C. After the mixture was stirred and turned clear, cupric acetate (0 or 0.3 equiv.) was added into the solution. The reaction mixture was cooled to 0-5° C. and lead (IV) acetate (1.5 equiv.) was added into the reaction mixture at 0-5° C. The mixture was then stirred at 0-5° C. for an hour and was then allowed to warm to 25-30° C. The reaction mixture was stirred at 25-30° C. for 16 hours until most compound 2 was consumed, which was monitored by LCMS. The resulting mixture was filtered through a short silica gel plug and the silica gel was washed with ethyl acetate (2×). The combined filtrate was diluted with ethyl acetate and water. After neutralized carefully with sodium bicarbonate powder to pH 7, the mixture was separated and the organic layer was washed with brine, dried over anhydrous sodium sulfate and concentrated in vacuo to give brown crude product, which was dissolved into DCM (3 L). The mixture was filtered through short silica gel plug eluting with DCM (3×) until compound 3 was totally collected. The collected solution was concentrated. To the residue was added MTBE and a white solid was precipitated at 25-30° C., which was collected by filtration. The solid was dried with nitrogen blowing at 25-30° C. for more than 16 hours to give pure compound 3 as a white solid. Or the brown crude product was purified by reversed phase flash chromatography or prep-HPLC to give pure compound 3 as a white solid.

### [2-({[(9H-fluoren-9-yl)methoxy]carbonyl}amino)acetamido]methyl acetate (3a)

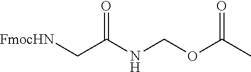

Following the general procedure (catalyzed with cupric acetate (0.3 equiv.)), compound 3a (1.3 kg, 60% yield) was obtained as a white solid. ESI m/z: 391 (M+Na)⁺. ¹H NMR (400 MHz, DMSO_{d6}) δ 8.96 (t, J=6.8 Hz, 1H), 7.90 (d, J=7.6 Hz, 2H), 7.72 (d, J=7.2 Hz, 2H), 7.59 (t, J=6.0 Hz, 1H), 7.43 (t, J=7.2 Hz, 2H), 7.34 (t, J=7.2 Hz, 2H), 5.10 (d, J=7.2 Hz, 2H), 4.36-4.19 (m, 3H), 3.66 (d, J=6.0 Hz, 2H), 2.00 (s, 3H) ppm.

### [(2S)-3-[(tert-butyldimethylsilyl)oxy]-2-({[(9H-fluoren-9-yl)methoxy]carbonyl}amino)propanamido]methyl acetate (3b)

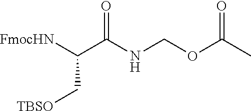

Following the general procedure without cupric acetate, compound 3b (0.19 g, 45% yield) was obtained as a white solid after purification by prep-HPLC (0-100% acetonitrile in aq. formic acid (0.1%)). ESI m/z: 535 (M+Na)⁺.

### benzyl (4S)-4-{[(acetyloxy)methyl]carbamoyl}-4-({[(9H-fluoren-9-yl)methoxy]carbonyl}amino)butanoate (3c)

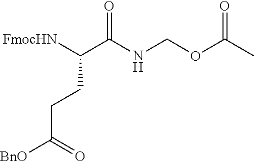

Following the general procedure without cupric acetate, compound 3c (0.20 g, 30% yield) was obtained as a white solid after purification by reversed phase flash chromatography (0-60% acetonitrile in aq. formic acid (0.1%)). ESI m/z: 553 (M+Na)⁺.

### [(2S)-6-azido-2-({[(9H-fluoren-9-yl)methoxy]carbonyl}amino)hexanamido]methyl acetate (3d)

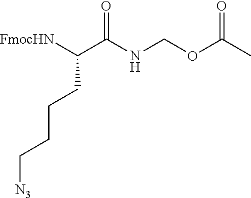

Following the general procedure without cupric acetate, compound 3d (0.57 g, 84% yield) was obtained as a white solid after purification by prep-HPLC (0-100% acetonitrile in aq. formic acid (0.1%)). ESI m/z: 488 (M+Na)⁺.

### [(2S)-2-({[(9H-fluoren-9-yl)methoxy]carbonyl}amino)-3-phenylpropanamido]methyl acetate (3e)

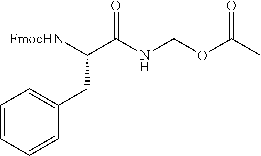

Following the general procedure without cupric acetate, compound 3c (0.36 g, 81% yield) was obtained as a white solid after purification by prep-HPLC (0-100% acetonitrile in aq. formic acid (0.1%)). ESI m/z: 481 (M+Na)⁺. ¹H NMR (400 MHz, DMSO) δ 9.13 (t, J=6.9 Hz, 1H), 7.88 (d, J=7.5 Hz, 2H), 7.71 (d, J=8.7 Hz, 1H), 7.67-7.58 (m, 2H), 7.46-7.36 (m, 2H), 7.35-7.23 (m, 6H), 7.19 (t, J=7.1 Hz, 1H), 5.18-5.04 (m, 2H), 4.32-4.21 (m, 1H), 4.21-4.07 (m, 3H), 3.05-2.73 (m, 2H), 2.00 (s, 3H) ppm.

### [2-({[(9H-fluoren-9-yl)methoxy]carbonyl}amino)-N-methylacetamido]methyl acetate (3f)

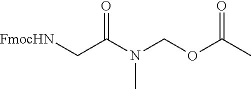

Following the general procedure without cupric acetate, compound 3f (1.65 g, 60% yield) was obtained as a white solid. ESI m/z: 405 (M+Na)⁺.

### [2-({[(9H-fluoren-9-yl)methoxy]carbonyl}amino)-2-methylpropanamido]methyl acetate (3g)

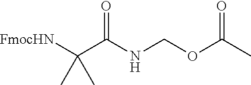

Following the general procedure (catalyzed with cupric acetate (0.3 equiv.)), compound 3g (0.36 g, 81% yield) was obtained as a white solid after purification by prep-HPLC (0-100% acetonitrile in aq. formic acid (0.1%)). ESI m/z: 481 (M+Na)⁺.

### [(2R)-2-({[(9H-fluoren-9-yl)methoxy]carbonyl}amino)-3-phenylpropanamido]methyl acetate (3h)

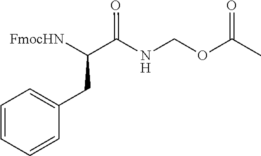

Following the general procedure (catalyzed with cupric acetate (0.3 equiv.)) (0.34 g, 80% yield) was obtained as a white solid after purification by prep-HPLC (0-100% acetonitrile in aq. formic acid (0.1%)). ESI m/z: 481 (M+Na)⁺.

General procedures of synthesis of compounds 4a-h

To a reaction flask were added 1,2-dichloroethane (0.10-0.15 M), compound 3 (1.0 equiv.), glycolic acid (0.6 equiv.), and pyridium p-toluenesulfonate (PPTS) (0.2 equiv.) at room temperature. The reaction mixture was heated to 45-50° C. and stirred for an hour, and to the hot solution was added twice glycolic acid (0.6 equiv. 2×) once per hour. The mixture was then stirred at 45-50° C. for 16 hours, which was monitored by LCMS. After cooled to 25-30° C., the precipitates were filtered and collected. The solid was dissolved into aqueous sodium bicarbonate (3%) obtaining the mixture with pH 7-8 at 5-10° C., which was washed with mixed solvent ethyl acetate and THF (v/v=1, 3×). To the aqueous layer was added MTBE at 5-10° C. and acidified with sat aq. citric acid to pH 3-4 to precipitate large amount of solid. The mixture was filtered, and the cake was washed with water (1×) and MTBE (2×), dried under nitrogen blow at 25-30° C. over 48 hours to give wet compound 4 (75% yield) as a white solid, which containing 3% water according to HNMR. The product was dried again in vacuum over 48 hours to give dry compound 4 (73% yield) as a white solid. Or the reaction mixture was directly purified by reversed phase flash chromatography or prep-HPLC to give pure compound 4 as a white solid.

### 2-{[2-({[(9H-Fluoren-9-yl)methoxy]carbonyl}amino)acetamido]methoxy}acetic acid (4a)

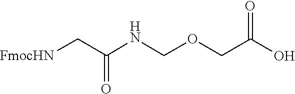

Following the general procedure, compound 4a (0.96 g, 80% yield) was obtained as a white solid. >99% in HPLC, ESI m/z: 407 (M+Na)⁺. ¹H NMR (400 MHz, DMSO_{d6}) δ 12.53 (br s, 1H), 8.72 (t, J=6.8 Hz, 1H), 7.90 (d, J=7.2 Hz, 2H), 7.72 (d, J=7.6 Hz, 2H), 7.59 (t, J=6.4 Hz, 1H), 7.42 (d, J=7.6 Hz, 2H), 7.33 (d, J=7.2 Hz, 2H), 4.60 (d, J=6.8 Hz, 2H), 4.31-4.18 (m, 3H), 3.98 (s, 2H), 3.62 (d, J=6.0 Hz, 2H) ppm.

### 2-{[(2S)-3-[(tert-butyldimethylsilyl)oxy]-2-({[(9H-fluoren-9-yl)methoxy]carbonyl}amino)propanamido]methoxy}acetic acid (4b)

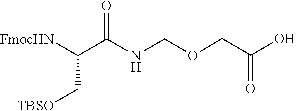

Following the general procedure, compound 4b (57 mg, 31% yield) was obtained as a yellow solid after purification by prep-HPLC (0-100% acetonitrile in aq. TFA (0.05%)). ESI m/z: 551 (M+Na)⁺.

### 2-{[(2S)-5-(benzyloxy)-2-({[(9H-fluoren-9-yl)methoxy]carbonyl}amino)-5-oxopentanamido]methoxy}acetic acid (4c)

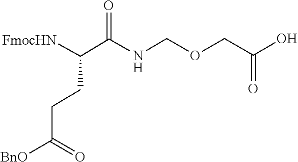

Following the general procedure, compound 4c (0.13 g, 65% yield) was obtained as a white solid after purification by prep-HPLC (0-90% acetonitrile in aq. formic acid (0.1%)). ESI m/z: 569 (M+Na)⁺.

### 2-{[(2S)-6-azido-2-({[(9H-fluoren-9-yl)methoxy]carbonyl}amino)hexanamido]methoxy}acetic acid (4d)

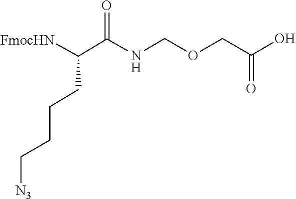

Following the general procedure, compound 4d (0.30 g, 51% yield) was obtained as a white solid after purification by prep-HPLC (5-95% acetonitrile in aq. ammonium bicarbonate (10 mM)). ESI m/z: 504 (M+Na)⁺.

### 2-{[(2S)-2-({[(9H-fluoren-9-yl)methoxy]carbonyl}amino)-3-phenylpropanamido]methoxy}acetic acid (4e)

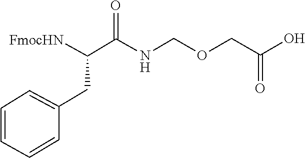

Following the general procedure, compound 4e (0.14 g, 38% yield) was obtained as a white solid after purification by reversed phase flash chromatography (0-25% acetonitrile in water). ESI m/z: 474 (M+Na)⁺.

### 2-{[2-({[(9H-fluoren-9-yl)methoxy]carbonyl}amino)-N-methylacetamido]methoxy}acetic acid (4f)

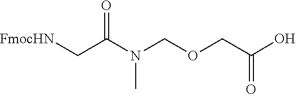

Following the general procedure, compound 4f (1.0 g, 50% yield) was obtained as a white solid after purification by reversed phase flash chromatography (0-100% acetonitrile in aq. TFA (0.01%)). ESI m/z: 421 (M+Na)⁺.

### 2-{[2-({[(9H-fluoren-9-yl)methoxy]carbonyl}amino)-2-methylpropanamido]methoxy}acetic acid (4g)

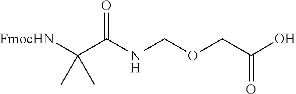

Following the general procedure, compound 4g (0.10 g, 40% yield) was obtained as a white solid after purification by reversed phase flash chromatography (0-100% acetonitrile in aq. TFA (0.01%)). ESI m/z: 435 (M+Na)⁺.

### 2-{[(2R)-2-({[(9H-fluoren-9-yl)methoxy]carbonyl}amino)-3-phenylpropanamido]methoxy}acetic acid (4h)

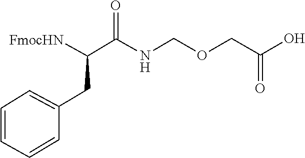

Following the general procedure, compound 4h (0.14 g, 38% yield) was obtained as a white solid after purification by reversed phase flash chromatography (0-100% acetonitrile in aq. TFA (0.01%)). ESI m/z: 474 (M+Na)⁺.

### Synthesis of 2-{[2-({[(9H-Fluoren-9-yl)methoxy]carbonyl}amino)acetamido]methoxy}acetic acid (4a)

The large scale synthesis of intermediate 4a is described in Scheme 8a.

### [2-({[(9H-Fluoren-9-yl)methoxy]carbonyl}amino)acetamido]methyl acetate (3a)

To a 10 L reaction flask were added THF (6.7 L) and Fmoc-Gly-Gly-OH (2a) (0.67 kg, 1.9 mol) at 25-30° C. To the obtained suspension was added pyridine (0.30 kg, 3.8 mol) at 25-30° C. After the mixture was stirred and turned clear, cupric acetate (0.10 kg, 0.57 mol) was added into the solution. The reaction mixture was cooled to 0-5° C. and lead(IV) acetate (1.7 kg, 2.8 mol) was added into the reaction mixture at 0-5° C. The mixture was then stirred at 0-5° C. for an hour and was then allowed to warm to 25-30° C. The reaction mixture was stirred at 25-30° C. for 16 hours until most of 2a was consumed, which was monitored by LCMS. The resulting mixture was filtered through a short silica gel plug (200 g) and the silica gel was washed with ethyl acetate (1 L×2). The combined filtrate was diluted with ethyl acetate (10 L) and water (10 L). After careful neutralization with sodium bicarbonate powder to pH 7, the mixture was separated and the organic layer was washed with brine (5 L×1), dried over anhydrous sodium sulfate, and concentrated in vacuo to give brown crude product, which was combined with the crude product from other two batches with similar LCMS (0.60 kg batch and 0.80 kg batch) and was dissolved into DCM (3 L). The mixture was filtered through short silica gel plug (0.30 kg) eluting with DCM (1 L×3) until compound 3a was totally collected. The collected solution was concentrated to 2 L. To the residue was added MTBE (3 L) and a white solid was precipitated at 25-30° C., which was collected by filtration. The solid was dried with nitrogen blowing at 25-30° C. for more than 16 hours to give pure compound 3a (1.3 kg, 60% yield) as a white solid. ESI m/z: 254 (M+H)⁺. ¹H NMR (400 MHz, DMSO_{d6}) δ 8.96 (t, J=6.8 Hz, 1H), 7.90 (d, J=7.6 Hz, 2H), 7.72 (d, J=7.2 Hz, 2H), 7.59 (t, J=6.0 Hz, 1H), 7.43 (t, J=7.2 Hz, 2H), 7.34 (t, J=7.2 Hz, 2H), 5.10 (d, J=7.2 Hz, 2H), 4.36-4.19 (m, 3H), 3.66 (d, J=6.0 Hz, 2H), 2.00 (s, 3H) ppm.

| | reactants | | reagents | | | | | | Solvent | Temp. | Time | | Product 3a | | |
|---|---|---|---|---|---|---|---|---|---|---|---|---|---|---|---|
| # | 2a | | Cu(OAc)₂ | | Pb(OAc)₄ | | Pyridine | | THF | (° C.) | (h) | purification | amount | Yield | purity |
| 1 | 0.67 | kg | 0.10 | kg, | 1.7 | kg | 0.30 | kg | 6.7 L | 0-5 | 1 | Filtration | 1.3 kg | 60% | 99% (LCMS) |
| | 1.9 | mol | 0.57 | mol | 2.8 | mol | 3.8 | mol | | 25-30 | 16 | through | | | 0.9 wt % MTBE |
| | | | (0.3 eq.) | | (1.5 eq.) | | (2.0 eq.) | | | | | silica gel | | | and 0.7 wt % |
| 2 | 0.60 | kg | 92 | g | 1.5 | kg | 0.27 | kg | 6 L | 0-5 | 1 | plug | | | DCM |
| | 1.7 | mol | 0.51 | mol | 3.4 | mol | 3.4 | mol | | 25-30 | 16 | | | | |
| | | | (0.3 eq.) | | (2.0 eq.) | | (2.0 eq.) | | | | | | | | |
| 3 | 0.80 | kg | 123 | g | 2.0 | kg | 0.36 | kg | 8 L | 0-5 | 1 | | | | |
| | 2.3 | mol | 0.68 | mol | 4.5 | mol | 4.6 | mol | | 25-30 | 16 | | | | |

| | | |
|---|---|---|
| (0.3 eq.) | (2.0 eq.) | (2.0 eq.) |

### 2-{[2-({[(9H-Fluoren-9-yl)methoxy]carbonyl}amino)acetamido]methoxy}acetic acid (4a)

To a 50 L jacked reaction flask were added 1,2-dichloroethane (19 L), compound 3a (0.96 kg, 2.6 mol), glycolic acid (0.12 kg, 1.6 mol), and pyridium p-toluenesulfonate (PPTS) (0.13 kg, 0.52 mol) successively at 20-25° C. The reaction mixture was stirred at 45-50° C. for an hour, and to the hot solution was added twice glycolic acid (0.12 kg, 1.6 mol) once per hour. The mixture was then stirred at 45-50° C. for 16 hours, which was monitored by LCMS. After cooled to 25-30° C., precipitates were filtered and collected, which were combined with other batches with similar LCMS (200 g, 1.25 kg). The combined solid was dissolved into aqueous sodium bicarbonate (0.44 kg in 15 L of water) providing the mixture with pH 7-8 at 5-10° C., which was washed with mixed solvent ethyl acetate and THF (v/v=1, 4.0 L×3). To the aqueous layer was added MTBE (5 L) at 5-10° C., and it was acidified with sat. aq. citric acid to pH 3-4 to precipitate large amount of solid. The mixture was filtered, and the cake was washed with water (1 L) and MTBE (1 L×2), dried under nitrogen blow at 25-30° C. over 48 hours to give compound 4a (1.1 kg, 75% yield) as a white solid (contains 3% of water according to HNMR). The product was dried again in vacuum over 48 hours to give dry compound 4a (0.96 kg, 97% recycled yield from wet product) as a white solid. >99% in HPLC, ESI m/z: 407 (M+Na)⁺. ¹H NMR (400 MHz, DMSO_{d6}) δ 12.53 (br s, 1H), 8.72 (t, J=6.8 Hz, 1H), 7.90 (d, J=7.2 Hz, 2H), 7.72 (d, J=7.6 Hz, 2H), 7.59 (t, J=6.4 Hz, 1H), 7.42 (d, J=7.6 Hz, 2H), 7.33 (d, J=7.2 Hz, 2H), 4.60 (d, J=6.8 Hz, 2H), 4.31-4.18 (m, 3H), 3.98 (s, 2H), 3.62 (d, J=6.0 Hz, 2H) ppm.

The product contained around 0.96% of unknown contaminate with M/Z=617 (positive mode). This side-product can be removed in the next step. The product C should be dried thoroughly as water will decrease the yield in the next step.

| | Reactants | | | reagents | | Solvent | Temp. | Time | | Product 4a | | |
|---|---|---|---|---|---|---|---|---|---|---|---|---|
| # | 3a | Glycolic acid | | PPTS | | DCE | (° C.) | (h) | purification | amount | Yield | purity |
| 1 | 0.20 kg | 0.074 | kg | 0.027 | kg | 4.0 L | 45-50 | 18 | Wash and | 0.96 kg | 75% | 99% |
| | 0.54 mol | 0.97 | mol | 0.11 | mol | | | | precipitated | | | (HPLC) |
| | | 1.8 | eq. | 0.20 | eq. | | | | | | | 0.5 wt % |
| 2 | 0.25 kg | 0.093 | kg | 0.034 | kg | 5.0 L | 45-50 | 18 | | | | ethyl |
| | 0.68 mol | 1.2 | mol | 0.14 | mol | | | | | | | acetate |
| | | 1.8 | eq. | (0.20 eq.) | | | | | | | | and |
| 3 | 0.96 kg | 0.12 kg × 3 | | 0.13 | kg | 19 L | 45-50 | 18 | | | | 0.6 wt % MTBE |
| | 2.6 mol | 4.7 | mol | 0.52 | mol | | | | | | | |
| | | 1.8 | eq. | 0.20 | eq. | | | | | | | |

General procedures of synthesis of compounds 5a-h

To a solution of compound 4 (1.1 equiv.) in DMF (5-8 mL per gram of 4) were added HATU (1.1 equiv.) and DIPEA (1.0 equiv.), and the reaction mixture was stirred at room temperature for 15 minutes. To the stirred solution was then added a mixture of Exatecan mesylate (1.0 equiv.) and DIPEA (2.0 equiv.) in DMF (10 mL per gram of Exatecan). The reaction mixture was stirred at room temperature for 4 hours, which was monitored by LCMS. The resulting mixture was diluted with ethyl acetate and washed with brine (2×). The organic layer was dried over anhydrous sodium sulfate, filtered, and concentrated in vacuo. The residue was co-evaporated with ethyl acetate (4×) in vacuo to provide crude product 5, which was added ethyl acetate. The suspension was refluxed for around 20 minutes until it turned clear and was then cooled to 25° C. naturally and stood for half an hour. The white precipitates were collected by filtration, washed with ethyl acetate (2×) and dried in vacuum to give 5 as a white solid. Or the crude product 5 was purified by reversed phase flash chromatography to give pure compound 5 as a solid.

### (9H-fluoren-9-yl)methyl N-({[({[(10S,23S)-10-ethyl-18-fluoro-10-hydroxy-19-methyl-5,9-dioxo-8-oxa-4,15-diazahexacyclo[14.7.1.0²,4.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamate (5a)

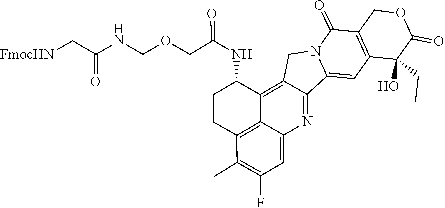

Following the general procedure, compound 5a (2.6 g, 87% yield) was obtained as a white solid ESI m/z: 802.3 (M+H)⁺. 95.2% in HPLC.

### (9H-fluoren-9-yl)methyl N-[(1S)-2-[(tert-butyldimethylsilyl)oxy]-1-{[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}ethyl]carbamate (5b)

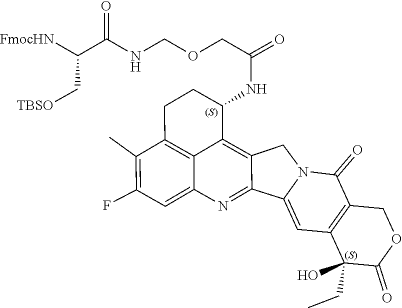

Following the general procedure, compound 5b (0.12 g, 53% yield) was obtained as a white solid after purification by reversed phase flash chromatography (0-100% acetonitrile in aq. TFA (0.05%)). ESI m/z: 946 (M+H)⁺.

### benzyl (4S)-4-{[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}-4-({[(9H-fluoren-9-yl)methoxy]carbonyl}amino)butanoate (5c)

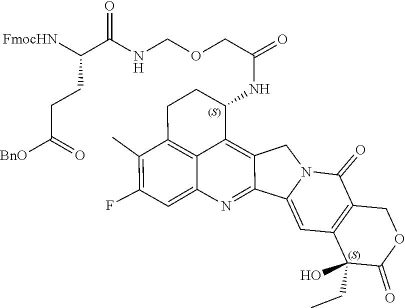

Following the general procedure, compound 5c (0.21 g, 74% yield) was obtained as a yellow solid after purification by reversed phase flash chromatography (0-70% acetonitrile in aq. formic acid (0.1%)). ESI m/z: 964 (M+H)⁺.

### (4S)-4-{[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}-4-({[(9H-fluoren-9-yl)methoxy]carbonyl}amino)butanoic acid (5ca)

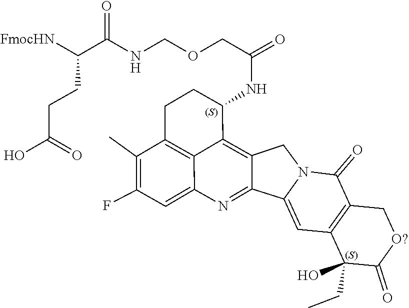

To a stirred solution of compound 5a (0.21 g, 0.22 mmol) in methanol (20 mL) was added palladium on carbon (36 mg, containing 10% palladium) under protection of nitrogen. The reaction mixture was stirred at room temperature under hydrogen atmosphere for 4 hours, which was monitored by LCMS. The mixture was filtered through Celite and the filtrate was concentrated in vacuo to give crude compound 5ca (0.10 g, 53% yield) as a yellow solid, which was used for the next step without further purification. ESI m/z: 874 (M+H)⁺.

### (9H-fluoren-9-yl)methyl N-[(1S)-3-carbamoyl-1-{[({[(10S,23S)-10-ethyl-18-fluoro-10-hydroxy-19-methyl-5,9-dioxo-8-oxa-4,15-diazahexacyclo[14.7.1.0²,4.0^{4,13}.0^{6,11}.0^{20,24}]tetracosa-1,6(11),12,14,16,18,20(24)-heptaen-23-yl]carbamoyl}methoxy)methyl]carbamoyl}propyl]carbamate (5cb)

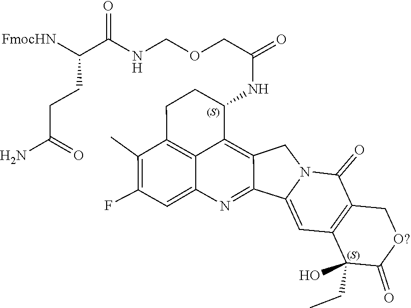

To a solution of compound 5ca (50 mg, 57 μmol) in DMF (1 mL) were added ammonium chloride (3.0 mg, 57 μmol), HATU (32 mg, 85 μmol) and DIPEA (22 mg, 0.17 mmol), and the reaction mixture was stirred at room temperature for 3 hours, which was monitored by LCMS. The resulting mixture was directly purified by reversed phase flash chromatography (0-100% acetonitrile in aq. formic acid (0.1%)) to give compound 5cb (40 mg, 81% yield) as a white solid. ESI m/z: 873 (M+H)⁺.

### (9H-fluoren-9-yl)methyl N-[(1S)-5-azido-1-{[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}pentyl]carbamate (5d)

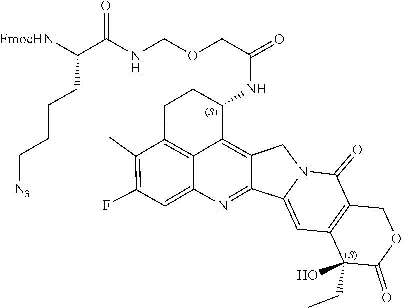

Following the general procedure, compound 5d (85 mg, 91% yield) was obtained as a white solid after purification by prep-HPLC (5-95% acetonitrile in aq. TFA (0.1%)). ESI m/z: 900 (M+H)⁺.

### (9H-fluoren-9-yl)methyl N-[(1S)-5-amino-1-{[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}pentyl]carbamate (5da)

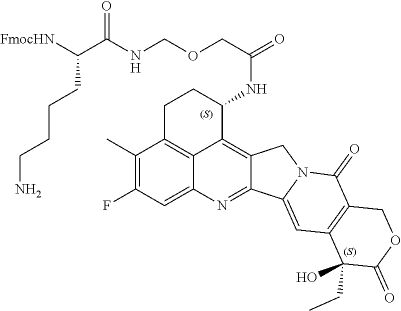

To a stirred solution of compound 5d (45 mg, 50 μmol) in methanol (20 mL) was added palladium on carbon (10 mg, containing 10% palladium) under protection of nitrogen. The reaction mixture was stirred at room temperature under hydrogen atmosphere for 2 hours, which was monitored by LCMS. The mixture was filtered through Celite and the filtrate was concentrated in vacuo and the residue was purified by prep-HPLC (5-95% acetonitrile in aq. TFA (0.03%)) to give compound 5da (38 mg, 86% yield) as a yellow solid. ESI m/z: 873 (M+H)⁺.

### (9H-fluoren-9-yl)methyl N-[(1S)-1-{[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}-2-phenylethyl]carbamate (5e)

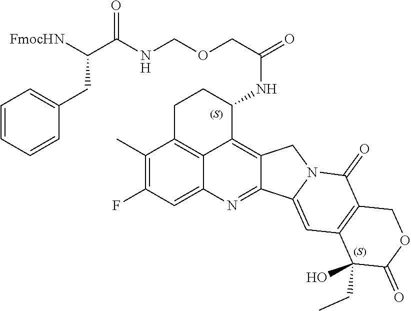

Following the general procedure, compound 5e (24 mg, 64% yield) was obtained as a white solid after purification by prep-HPLC (5-95% acetonitrile in aq. TFA (0.1%)). ESI m/z: 892 (M+H)⁺.

### (9H-fluoren-9-yl)methyl N-({[({[(10S,23S)-10-ethyl-18-fluoro-10-hydroxy-19-methyl-5,9-dioxo-8-oxa-4,15-diazahexacyclo[14.7.1.0²,4.0^{4,13}.0^{6,11}.0^{20,24}]tetracosa-1,6(11),12,14,16,18,20(24)-heptaen-23-yl]carbamoyl}methoxy)methyl](methyl)carbamoyl}methyl)carbamate (5f)

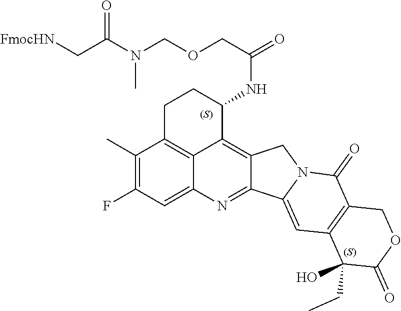

Following the general procedure, compound 5f (50 mg, 61% yield) was obtained as a white solid after purification by prep-HPLC (5-95% acetonitrile in aq. TFA (0.01%)). ESI m/z: 816 (M+H)⁺.

### (9H-fluoren-9-yl)methyl N-(1-{[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}-1-methylethyl)carbamate (5g)

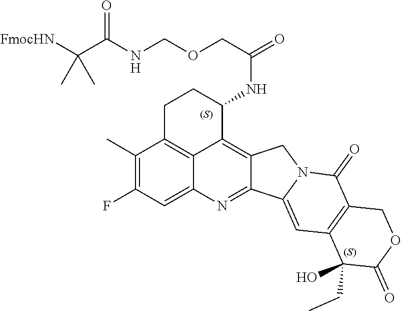

Following the general procedure, compound 5g (0.17 g, 61% yield) was obtained as a white solid after purification by reversed phase flash chromatography (0-70% acetonitrile in aq. TFA (0.01%)). ESI m/z: 830 (M+H)⁺.

### (9H-fluoren-9-yl)methyl N-[(1R)-1-{[({[(10S,23S)-10-ethyl-18-fluoro-10-hydroxy-19-methyl-5,9-dioxo-8-oxa-4,15-diazahexacyclo[14.7.1.0²,4.0^{4,13}.0^{6,11}.0^{20,24}]tetracosa-1,6(11),12,14,16,18,20(24)-heptaen-23-yl]carbamoyl}methoxy)methyl]carbamoyl}-2-phenylethyl]carbamate (5h)

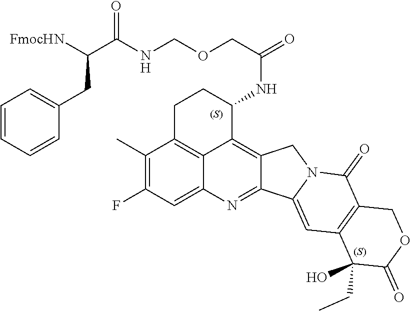

Following the general procedure, compound 5h (92 mg, 73% yield) was obtained as a yellow solid after purification by prep-HPLC (5-95% acetonitrile in aq. TFA (0.1%)). ESI m/z: 892 (M+H)⁺.

### Payloads (ProDXds)

### General Procedure of De-Fmoc to Obtain Payloads (ProDXds)

To a solution of compound 5 (1.0 equiv.) in THF (20 mL per gram of 5) was added diethylamine (2 mL per gram of 5), and the reaction mixture was stirred at room temperature for 2-48 hours until Fmoc was totally removed according to LCMS. The volatiles were removed thoroughly in vacuo and the residue was diluted with water (5 mL). The aqueous mixture was adjusted to pH 2 with the addition of aq. TFA (10%) and was washed with MTBE (20 mL×2). The aqueous layer was then stirred at room temperature for 16 hours until the ring-open form turned to lactone form, which was monitored by LCMS. The resulting aqueous mixture was lyophilized to give crude payloads, which was purified by reversed phase flash chromatography (0-100% acetonitrile in aq. TFA (0.03%)) to give pure payloads (TFA salt) as a solid or prep-HPLC (5-95% acetonitrile in aq. formic acid (0.1%)) to give pure payloads (free base) as a solid.

### P1

### 2-Amino-N-[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]acetamide (P1)

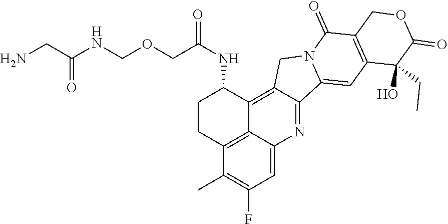 [SMILES: CC[C@@]1(O)C(=O)OCc2c1cc1n(c2=O)Cc2c-1nc1cc(F)c(C)c3c1c2[C@@H](NC(=O)COCNC(=O)CN)CC3]

Following the general procedure, P1 (1.4 g, 77% yield) was obtained as a light-yellow solid. ESI m/z: 580.3 (M+H)⁺. ¹H NMR (400 MHz, DMSO_{d6}) δ 9.20 (t, J=6.4 Hz, 1H), 8.59 (d, J=9.2 Hz, 1H), 8.07 (br s, 3H), 7.80 (d, J=11.2 Hz, 1H), 7.33 (s, 1H), 6.57 (s, 1H), 5.64-5.57 (m, 1H), 5.43 (s, 2H), 5.30-5.09 (m, 2H), 4.77-4.69 (m, 2H), 4.07 (s, 2H), 3.67 (s, 2H), 3.28-3.09 (m, 2H), 2.40 (s, 3H), 2.26-2.12 (m, 2H), 1.93-1.80 (m, 2H), 0.88 (t, J=7.2 Hz, 3H) ppm. ¹⁹F NMR (376 MHz, DMSO_{d6}) δ −73 (TFA), −111 (Ar-F) ppm.

### P2

### (2S)-2-amino-N-[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]-3-hydroxypropanamide (P2)

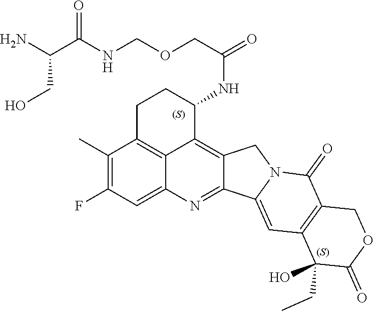

To a solution of compound 5b (0.12 g, 0.12 mmol) in DMF (1 mL) was added diethylamine (0.1 mL), and the mixture was stirred at room temperature for 2 hours until Fmoc was totally removed, which was monitored by LCMS. The resulting mixture was directly purified by reserved phase flash chromatography (0-100% acetonitrile in aq. TFA (0.05%)) to give de-Fmoc-product (68 mg, ESI m/z: 724 (M+H)⁺) as a yellow solid, which was dissolved in DMF (1 mL). To the solution was added cesium fluoride (31 mg, 0.20 mmol) at 0° C. The mixture was stirred at room temperature for an hour, which was monitored by LCMS. The mixture was separated by reversed phase flash chromatography (0-100% acetonitrile in aq. TFA (0.05%)) to give P2 (17 mg, 22% yield) as a white solid. ESI m/z: 610 (M+H)⁺. ¹H NMR (400 MHz, DMSO_{d6}) δ 9.26 (t, J=6.4 Hz, 1H), 8.58 (d, J=8.8 Hz, 1H), 8.14 (s, 2H), 7.79 (d, J=10.8 Hz, 1H), 7.33 (s, 1H), 6.56 (s, 1H), 5.60-5.53 (m, 2H), 5.42 (s, 2H), 5.20-5.18 (m, 2H), 4.80-4.76 (m, 1H), 4.67-4.63 (m, 1H), 4.05 (s, 2H), 3.90-3.89 (m, 1H), 3.77-3.76 (m, 2H), 3.27-3.14 (m, 2H), 2.39 (s, 3H), 2.17-2.16 (m, 2H), 1.90-1.83 (m, 2H), 0.87 (t, J=6.8 Hz, 3H) ppm. ¹⁹F NMR (376 MHz, DMSO_{d6}) δ −74 (TFA), −111 (Ar-F) ppm.

### P3

### (2S)-2-amino-N-[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]pentanediamide (P3)

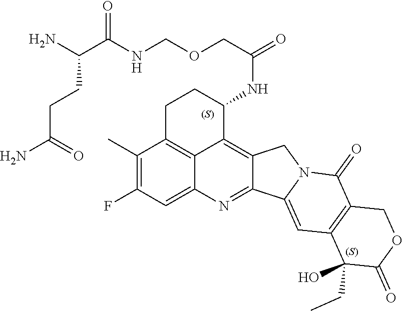

Following the general procedure, P3 (20 mg, 49% yield) was obtained as a light-yellow solid. ESI m/z: 651 (M+H)⁺. ¹H NMR (400 MHz, DMSO_{d6}) δ 8.95-8.80 (m, 1H), 8.80 (d, J=8.4 Hz, 1H), 8.18 (s, 1H), 7.81 (d, J=11.2 Hz, 1H), 7.31 (s, 1H), 7.29-7.20 (m, 1H), 6.73 (s, 1H), 6.52 (s, 1H), 5.65-5.56 (m, 1H), 5.42 (s, 2H), 5.21 (s, 2H), 4.63 (br s, 2H), 4.01 (s, 1H), 3.25-3.13 (m, 2H), 3.06-2.90 (m, 2H), 2.40 (s, 3H), 2.25-2.05 (m, 4H), 1.93-1.74 (m, 3H), 1.64-1.50 (m, 1H), 0.87 (t, J=6.8 Hz, 1H) ppm. ¹⁹F NMR (376 MHz, DMSO_{d6}) δ −111 (Ar-F) ppm.

### P4

### (4S)-4-amino-4-{[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}butanoic acid (P4)

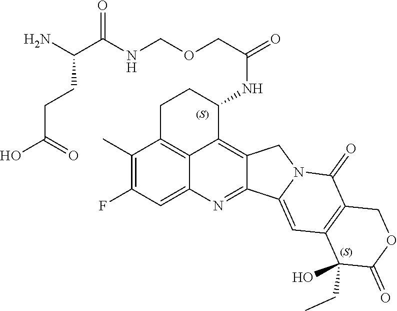

Following the general procedure, P4 (20 mg, 49% yield) was obtained as a light-yellow solid. ESI m/z: 652 (M+H)⁺. ¹H NMR (400 MHz, DMSO_{d6}) δ 8.95-8.82 (m, 1H), 8.55 (d, J=9.2 Hz, 1H), 8.30 (s, 1H), 7.79 (d, J=11.2 Hz, 1H), 7.31 (s, 1H), 5.65-5.56 (m, 1H), 5.42 (s, 2H), 5.21 (s, 2H), 4.62 (br s, 2H), 4.00 (s, 2H), 3.25-3.10 (m, 4H), 3.06-2.90 (m, 2H), 2.32 (s, 3H), 2.27-2.12 (m, 4H), 1.92-1.71 (m, 3H), 1.60-1.50 (m, 1H), 0.87 (t, J=7.2 Hz, 3H) ppm. ¹⁹F NMR (376 MHz, DMSO_{d6}) δ −111 (Ar-F) ppm.

### P5

### (2S)-2,6-diamino-N-[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]hexanamide (P5)

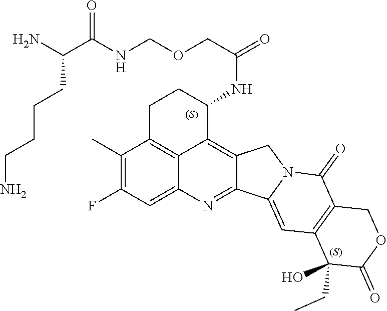

Following the general procedure, P5 (13 mg, 43% yield) was obtained as a white solid. ESI m/z: 651 (M+H)⁺. ¹H NMR (400 MHz, DMSO_{d6}) δ 9.33 (t, J=6.5 Hz, 1H), 8.61 (d, J=8.8 Hz, 1H), 8.18 (s, 3H), 7.81 (d, J=10.9 Hz, 1H), 7.72 (s, 2H), 7.34 (s, 1H), 6.57 (s, 1H), 5.63-5.57 (m, 1H), 5.43 (s, 2H), 5.26-5.15 (m, 2H), 4.79-4.67 (m, 2H), 4.11-4.01 (m, 2H), 3.86-3.80 (m, 1H), 3.25-3.10 (m, 2H), 2.81-2.72 (m, 2H), 2.40 (s, 3H), 2.22-2.13 (m, 2H), 1.92-1.83 (m, 2H), 1.80-1.71 (m, 2H), 1.60-1.50 (m, 2H), 1.40-1.31 (m, 2H), 0.88 (t, J=7.3 Hz, 3H) ppm. ¹⁹F NMR (376 MHz, DMSO_{d6}) δ −74 (TFA), −111 (Ar-F) ppm.

### P6

### (2S)-2-amino-6-azido-N-[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]hexanamide (P6)

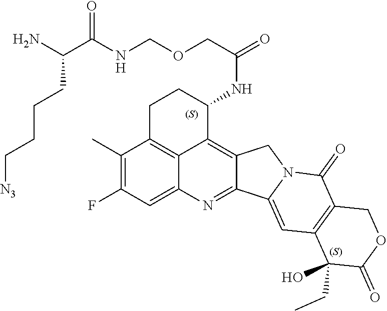

Following the general procedure, P6 (27 mg, 89% yield) was obtained as a white solid. ESI m/z: 677 (M+H)⁺. ¹H NMR (400 MHz, DMSO_{d6}) δ 8.92-8.78 (m, 1H), 8.57 (d, J=8.8 Hz, 1H), 8.23 (s, 1H), 7.77 (d, J=10.9 Hz, 1H), 7.30 (s, 1H), 6.54 (s, 1H), 5.66-5.54 (m, 1H), 5.41 (s, 2H), 5.19 (s, 2H), 4.60 (d, J=1.8 Hz, 2H), 4.00 (s, 2H), 3.26 (t, J=6.8 Hz, 3H), 3.21-3.11 (m, 3H), 2.38 (s, 3H), 2.24-2.12 (m, 2H), 1.92-1.80 (m, 2H), 1.54-1.41 (m, 3H), 1.36-1.24 (m, 3H), 0.87 (t, J=7.3 Hz, 3H) ppm. ¹⁹F NMR (376 MHz, DMSO_{d6}) 6-111 (Ar-F) ppm.

### P7

### (2S)-2-amino-N-[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]-3-phenylpropanamide (P7)

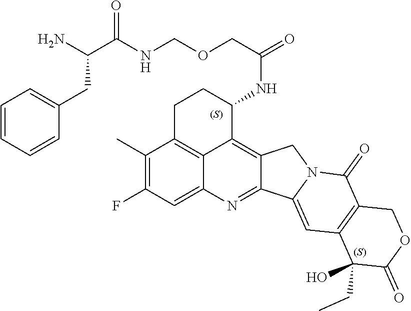

Following the general procedure, P7 (15 mg, 62% yield) was obtained as a white solid. ESI m/z: 670 (M+H)⁺. ¹H NMR (400 MHz, DMSO_{d6}) δ 8.86-8.77 (m, 1H), 8.56 (d, J=8.8 Hz, 1H), 8.29 (s, 1H), 7.78 (d, J=11.0 Hz, 1H), 7.29 (s, 1H), 7.21 (t, J=7.2 Hz, 2H), 7.12 (t, J=8.5 Hz, 3H), 6.52 (s, 1H), 5.64-5.56 (m, 1H), 5.45-5.34 (m, 2H), 5.25-5.12 (m, 2H), 4.58 (s, 2H), 3.96 (s, 2H), 3.25-3.08 (m, 4H), 2.85-2.70 (m, 1H), 2.39 (s, 3H), 2.28-2.08 (m, 3H), 1.87-1.76 (m, 2H), 0.84 (t, J=7.2 Hz, 3H) ppm. ¹⁹F NMR (376 MHz, DMSO_{d6}) 6-111 (Ar-F) ppm.

### P9

### 2-amino-N-[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]-N-methylacetamide (P9)

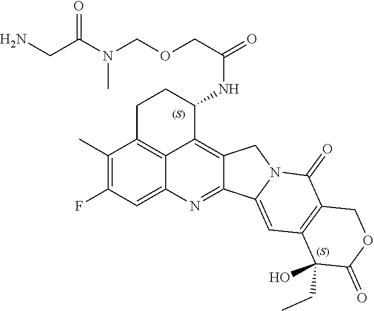

Following the general procedure, P9 (22 mg, 60% yield) was obtained as a white solid. ESI m/z: 594 (M+H)⁺. ¹H NMR (400 MHz, DMSO_{d6}) δ 8.70 (d, J=8.9 Hz, 0.5H), 8.61 (d, J=8.9 Hz, 0.5H), 8.07 (s, 2H), 7.82-7.78 (m, 1H), 7.33 (d, J=1.8 Hz, 1H), 6.55 (d, J=2.4 Hz, 1H), 5.67-5.53 (m, 1H), 5.43 (s, 2H), 5.29-5.10 (m, 2H), 4.96-4.87 (m, 2H), 4.20-3.90 (m, 4H), 3.19 (d, J=6.6 Hz, 2H), 3.02 (s, 1.5H), 2.99 (s, 1.5H), 2.40 (s, 3H), 2.19-2.17 (m, 2H), 1.93-1.81 (m, 2H), 0.88 (t, J=7.2 Hz, 3H) ppm. ¹⁹F NMR (376 MHz, DMSO_{d6}) δ −73 (TFA), −111 (Ar-F) ppm.

### P11

### 2-amino-N-[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]-2-methylpropanamide (P11)

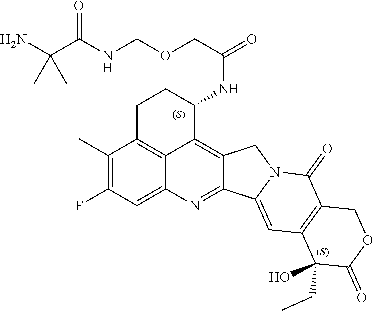

Following the general procedure, P11 (15 mg, 62% yield) was obtained as a white solid. ESI m/z: 608 (M+H)⁺. ¹H NMR (400 MHz, DMSO_{d6}) δ 9.17-9.14 (m, 1H), 8.56 (d, J=8.0 Hz, 1H), 8.17 (s, 2H), 7.80 (d, J=8.0 Hz, 1H), 7.33 (s, 1H), 6.56 (s, 1H), 5.63-5.68 (m, 1H), 5.42 (s, 2H), 5.21 (s, 2H), 4.73 (d, J=4.0 Hz, 2H), 4.04 (s, 2H), 3.24-3.13 (m, 2H), 2.40 (s, 3H), 2.18 (d, J=4.0 Hz, 2H), 1.89-1.83 (m, 2H), 1.475 (s, 3H), 1.473 (s, 3H), 0.89-0.86 (m, 3H) ppm. ¹⁹F NMR (376 MHz, DMSO_{d6}) δ −73 (TFA), −111 (Ar-F) ppm.

### P12

### (2R)-2-amino-N-[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]-3-phenylpropanamide (P12)

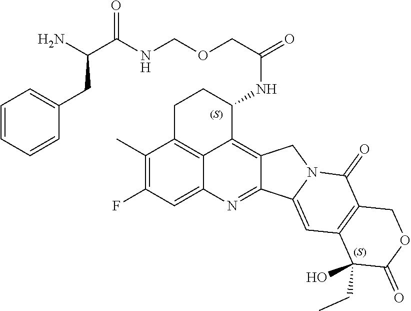

Following the general procedure, P12 (36 mg, 52% yield, TFA salt) was obtained as a white solid. ESI m/z: 670 (M+H)⁺. ¹H NMR (400 MHz, DMSO_{d6}) δ 9.29-9.26 (m, 1H), 8.55 (d, J=8.0 Hz, 1H), 8.19 (s, 3H), 7.80 (d, J=12 Hz, 1H), 7.33-7.23 (m, 6H), 6.55 (s, 1H), 5.62-5.57 (m, 1H), 5.45-5.35 (m, 2H), 5.20 (s, 2H), 4.69 (d, J=8.0 Hz, 1H), 4.07-3.96 (m, 3H), 3.11-2.96 (m, 3H), 2.40 (s, 3H), 2.21-2.16 (m, 2H), 1.92-1.82 (m, 2H), 1.27-1.23 (m, 1H), 0.88 (t, J=8.0 Hz, 3H) ppm. ¹⁹F NMR (376 MHz, DMSO_{d6}) δ −73 (TFA), −111 (Ar-F) ppm.

### (9H-fluoren-9-yl)methyl N-({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamate (Fmoc-ProDXd) (5a)

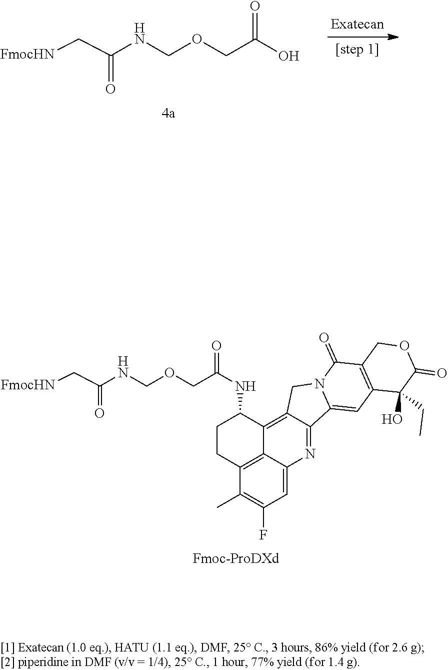

Exatecan is commercially available.

To a yellow solution of intermediate 4a (9.55 g, 24.86 mmol) in dry DMF (60 mL) were added HATU (9.45 g, 24.86 mmol) and DIPEA (2.91 g, 22.6 mmol), and the mixture was stirred at 25° C. for 15 minutes. To the reaction mixture was then added a mixed solution of Exatecan mesylate (12.0 g, 22.6 mmol) and DIPEA (5.82 g, 45.2 mmol) in dry DMF (60 mL). The reaction solution was stirred at 25° C. for 4 hours until Exatecan mesylate was consumed, which was monitored by LCMS. The resulting solution was diluted with ethyl acetate (0.90 L) and washed with brine (180 mL×2). The organic phase was dried over anhydrous sodium sulfate and concentrated in vacuo. The residue was co-evaporated with ethyl acetate (180 mL×4) in vacuo and the residue (50 g) was dissolved in ethyl acetate (400 mL). The suspension was refluxed for 20 minutes until it turned clear. And the solution was stood and white solid precipitated. The suspension was refluxed for an hour again and it was then cooled to 25° C. naturally and stood for half an hour. The white precipitates were collected by filtration and dried in vacuo to give compound Fmoc-proDxd (5a)(14.2 g, 78.3% yield, >99% purity) as a white solid. The filtrate was concentrated and purified by C18 column to provide (1.6 g, 8.8% yield, 97% purity). ESI m/z: 802.2 (M+H)⁺. ¹HNMR (400 MHz, DMSO_{d6}): δ 8.79 (t, J=6.4 Hz, 1H), 8.50 (d, J=9.6 Hz, 1H), 7.88 (d, J=7.6 Hz, 2H), 7.77 (d, J=10.8 Hz, 1H), 7.68 (d, J=7.2 Hz, 2H), 7.56 (t, J=6.0 Hz, 1H), 7.39 (t, J=7.6 Hz, 2H), 7.34 (s, 1H), 7.34-7.27 (m, 2H), 6.62-6.45 (m, 1H), 5.66-5.34 (m, 3H), 5.25-5.16 (m, 2H), 4.70-4.57 (m, 2H), 4.30-4.12 (m, 3H), 4.01 (s, 2H), 3.74-3.54 (m, 2H), 3.25-3.05 (m, 2H), 2.37 (s, 3H), 2.24-2.13 (m, 2H), 1.92-1.80 (m, 2H), 0.84 (t, J=7.6 Hz, 3H) ppm. ¹⁹FNMR (376 MHz, DMSO_{d6}) δ −111.33 ppm.

| | Raw material (Equivalent/Volume) | | | | | | | | | | | | | | | |
|---|---|---|---|---|---|---|---|---|---|---|---|---|---|---|---|---|
| | Exatecan | | Cpd C | | HATU | | DIPEA | | | Condition | | Purification | | | | |
| En- | mesylate | | (1.1 | | (1.1 | | (3.0 | | | Temp | Time | C18 column | Product | | | |
| try | (1.0 eq.) | | equiv.) | | equiv.) | | equiv.) | | DMF | (° C.) | (hr) | purification | Amount | Purity | Yield | Property |
| 1 | 1.0 | g | 0.80 | g | 0.79 | g | 0.73 | g | 15 ml | 25 | 16 | C18 column | 1.4 g | 97% | 93% | white |
| | | | | | | | | | | | | purification | | | | solid |
| 2 | 2.0 | g | 1.6 | g | 1.6 | g | 1.5 | g | 30 ml | 25 | 16 | C18 column | 2.6 g | 99% | 87% | white |
| | | | | | | | | | | | | purification | | | | solid |
| 3 | 2.0 | g | 1.6 | g | 1.6 | g | 1.5 | g | 30 ml | 25 | 16 | C18 column | 2.7 g | 97% | 90% | white |
| | | | | | | | | | | | | purification | | | | solid |
| 4 | 12 | g | 9.55 | g | 9.45 | g | 8.74 | g | 180 mL | 25 | 4 | Precipitation | 14.2 g | >99% | 78.3% | white |
| | 22.6 | mmol | 24.86 | mmol | 24.86 | mmol | 67.8 | mmol | | | | Mother | 1.6 | 97% | 8.8% | solid |

solution
purified
by C18
column

### Example 6. Exemplary Synthesis of ProDXds from DXd (Scheme 2B)

P1 (CP1190) synthesized from DXd

### 2-Amino-N-[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]acetamide (P1)

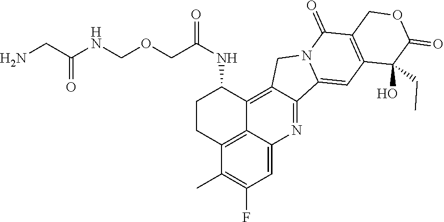 [SMILES: CC[C@@]1(O)C(=O)OCc2c1cc1n(c2=O)Cc2c-1nc1cc(F)c(C)c3c1c2[C@@H](NC(=O)COCNC(=O)CN)CC3]

To a solution of DXd (62 mg, 0.13 mmol) in THF (HPLC grade, 5 mL) were added compound 3a (0.23 g, 0.63 mmol) and 4 Å molecular sieves, and the mixture was stirred at room temperature for 5 minutes. To the mixture was then added Tf₂NH (0.18 g, 0.63 mmol) and the reaction mixture was stirred at room temperature for 10 minutes. Although there's still DXd remaining according to LCMS, the reaction was quenched by aq. TFA (0.1%, 0.05 mL). The mixture was directly separated by reversed phase flash chromatography (0-100% acetonitrile in aq. TFA (0.1%)) to give DXd (40 mg, 65% recovered yield) and 5a (Fmoc-P1) (31 mg, ESI m/z: 803 (M+H)⁺) as a light-yellow solid, which was dissolved in DMF (1 mL). To the 5a solution was added diethylamine (0.1 mL) and the reaction mixture was stirred at room temperature for an hour until Fmoc was totally removed according to LCMS. The resulting mixture was directly purified by prep-HPLC (5-95% acetonitrile in aq. TFA (0.1%)) to give P1 (15 mg, 17% yield, TFA salt) as a light-yellow solid. ESI m/z: 580.3 (M+H)⁺.

### Example 7. Exemplary Synthesis of di-aminoacid-ProDXd (Scheme 2C)

### P8

### 2-amino-N-({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)acetamide (P8)

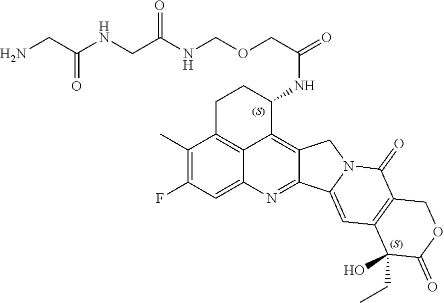

To a solution of N-Fmoc-glycine (16.2 mg, 0.054 mmol) in DMF (1 mL) were added HATU (30.9 mg, 0.081 mmol) and DIPEA (21 mg, 0.108 mmol), and the reaction mixture was stirred at room temperature for 15 minutes. To the stirred mixture was added compound P1 (30 mg, 0.054 mmol, TFA salt), and the reaction mixture was stirred at room temperature for an hour, which was monitored by LCMS. To the resulting mixture was then added diethylamine (1 mL) and the mixture was stirred at room temperature for an hour until Fmoc was totally removed according to LCMS. The volatiles were removed in vacuo and the residual mixture was directly separated by prep-HPLC (5-95% acetonitrile in aq. TFA (0.1%)) to give P8 (11 mg, 31% yield, TFA salt) as a light-yellow solid. ESI m/z: 637 (M+H)⁺. ¹H NMR (400 MHz, DMSO_{d6}) 68.88 (t, J=6.4 Hz, 1H), 8.65 (t, J=5.6 Hz, 1H), 8.54 (d, J=8.8 Hz, 1H), 8.01 (br s, 3H), 7.80 (d, J=10.4 Hz, 1H), 7.31 (s, 1H), 6.55 (s, 1H), 5.62-5.56 (m, 1H), 5.42 (s, 2H), 5.19 (s, 2H), 4.65 (d, J=6.4 Hz, 2H), 4.01 (s, 2H), 3.86 (d, J=5.6 Hz, 2H), 3.67 (s, 2H), 3.24-3.09 (m, 2H), 2.39 (s, 3H), 2.24-2.11 (m, 2H), 1.94-1.79 (m, 2H), 0.87 (t, J=7.2 Hz, 3H) ppm. ¹⁹F NMR (376 MHz, DMSO_{d6}) δ −73 (TFA), −111 (Ar-F) ppm.

### Example 8. Synthesis of 2-[2-({[(9H-fluoren-9-yl)methoxy]carbonyl}amino)ethanesulfonamido]acetic acid (2i)

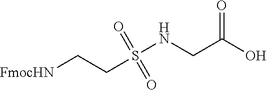

To a stirred solution of tert-butyl glycinate (0.42 g, 2.5 mmol) in DMF (8 mL) were added N-Fmoc-2-aminoethanesulfonyl chloride (0.83 g, 2.3 mmol) and DIPEA (0.88 g, 6.8 mmol) at 0° C. The reaction was stirred at room temperature for 2 hours, which was monitored by LCMS. The resulting mixture was directly purified by reversed phase flash chromatography (0-60% acetonitrile in aq. TFA (0.1%)) to give a white solid (0.23 g, ESI m/z: 483 (M+Na)+) which was dissolved in DCM (10 mL). To the solution was added TFA (1 mL), and the reaction mixture was stirred at room temperature for 8 hours, which was monitored by LCMS. The mixture was concentrated in vacuo to give compound 2i (0.19 g, 21% yield) as a yellow solid, which was used for the next step without further purification. ESI m/z: 427 (M+Na)+, 405 (M+H)⁺.

### Synthesis of Linker-Payloads

### Example 9. Exemplary Synthesis of Linker-Payloads by Route 1 (Scheme 4A)

### LP1 (M2980) Synthesized from Exatecan Reacted with Intermediate Fa

### {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamate (LP1)

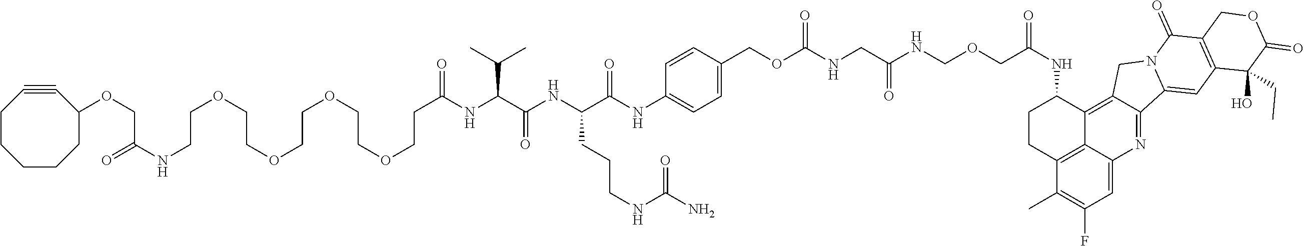 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

To a stirred solution of compound F (see example 26) (48 mg, 49 μmol, 88% purity) in DMF (1 mL) were added HATU (20 mg, 54 μmol) and DIPEA (13 mg, 98 μmol) at 25° C., and the mixture was stirred at 25° C. for 15 minutes. To a mixture of Exatecan mesylate (26 mg, 49 μmol) in DMF (0.8 mL) was added DIPEA (6.3 mg, 49 μmol) at 0° C., and the Exatecan solution was stirred at 25° C. for 15 minutes. The two solutions were mixed at 25° C. and the mixture was stirred at 25° C. for 16 hours, which was monitored by LCMS. The resulting mixture was directly purified by reversed phase flash chromatography (5-95% acetonitrile in aq. TFA (0.1%) in 60 minutes, flow rate 75 mL/min.) to give LP1 (35 mg, 51% yield, 98% purity in HPLC) as a white solid. ESI m/z: 1396 (M+H)⁺. ¹H NMR (400 MHz, DMSO_{d6}) δ 10.00 (s, 1H), 8.80 (t, J=6.4 Hz, 1H), 8.51 (d, J=8.8 Hz, 1H), 8.13 (d, J=7.6 Hz, 1H), 7.90 (d, J=8.8 Hz, 1H), 7.79 (d, J=10.8 Hz, 1H), 7.65-7.50 (m, 3H), 7.43 (t, J=6.0 Hz, 1H), 7.31 (s, 1H), 7.27 (d, J=8.8 Hz, 2H), 6.54 (s, 1H), 5.98 (t, J=5.2 Hz, 1H), 5.63-5.57 (m, 1H), 5.41 (s, 4H), 5.21 (s, 2H), 4.92 (s, 2H), 4.62 (d, J=6.4 Hz, 2H), 4.43-4.33 (m, 1H), 4.31-4.17 (m, 2H), 4.01 (s, 2H), 3.86 (d, J=14.4 Hz, 1H), 3.75 (d, J=14.8 Hz, 1H), 3.67-3.54 (m, 3H), 3.53-3.46 (m, 12H), 3.44-3.39 (m, 2H), 3.27-3.10 (m, 4H), 3.06-2.90 (m, 2H), 2.47-2.32 (m, 5H), 2.26-1.64 (m, 14H), 1.63-1.52 (m, 3H), 1.47-1.32 (m, 3H), 0.90-0.80 (m, 9H) ppm. ¹³C NMR (100 MHz, DMSO_{d6}) δ 174.77, 169.55, 168.95, 167.68, 167.16,161.10, 158.63, 157.39, 154.93, 150.50, 148.30, 146.11, 143.38, 138.89, 137.01, 134.57, 129.97, 123.39, 122.06, 121.87, 119.88, 117.41, 117.30, 108.08, 99.18, 95.12, 90.49, 70.73, 70.55, 68.18, 68.11, 67.89, 67.20, 66.23, 65.31, 63.69, 55.98, 51.52, 47.95, 43.14, 41.97, 40.04, 36.42, 34.34, 32.25, 28.95, 28.72, 27.63, 26.25, 25.18, 24.23, 22.21, 18.38, 17.55, 16.47, 9.22, 6.10 ppm. ¹⁹F NMR (376 MHz, DMSO_{d6}) δ −74.132 (0.3F, CF₃CO₂H), −111.314 (1F) ppm.

### Example 10. Exemplary Synthesis of Linker-payloads by route 2 (Scheme 4B)

### LP1 (M2980) synthesized from DXd reacted with intermediate Ea {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamate (LP1)

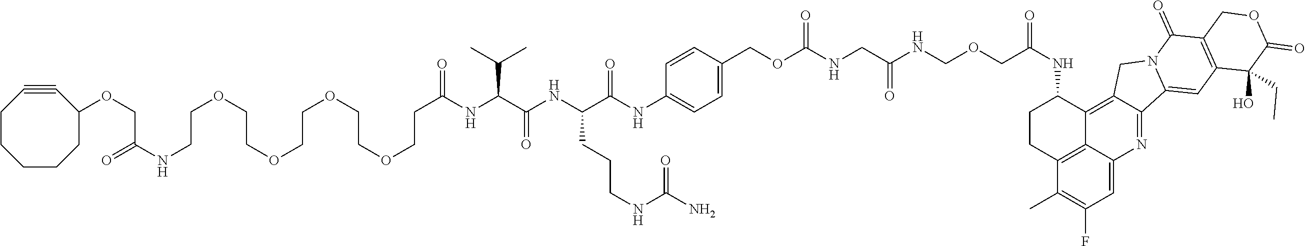 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

To a stirred mixture of intermediate Ea (see example 25) (50 mg, 52 μmol) in dry THF (5 mL) were added DXd (26 mg, 52 μmol) and 4A molecular sieves, and the mixture was stirred at 20° C. for 5 minutes before the addition of trifluoromethanesulfonimide (73 mg, 0.25 mmol). The reaction mixture was stirred at 20° C. for 10 minutes until intermediate Ea was mostly consumed, which was monitored by LCMS. The 4A molecular sieves was removed by filtration and the filtrate was concentrated in vacuo. The residue was purified by reversed phase flash chromatography (5-95% acetonitrile in aq. TFA (0.1%)) to give LP1 (29 mg, 39% yield) as a light-yellow solid. ESI m/z: 699.1 (M/2+H)⁺.

### Example 11. Synthesis of Linker-payloads by route 3a (Scheme 4C)

General procedure of linker-ProDXds by route 3a.

To a solution of intermediate D (1.0-1.2 equiv.) in DMF (0.15 mM) were added HOBt (0.5 equiv.) or HOAt (0.5 equiv.), DIPEA (3.0 equiv.) and payload (1.0 equiv.), and the reaction mixture was stirred at room temperature for 2 hours, which was monitored by LCMS. The resulting mixture was directly purified by reversed phase flash chromatography to give linker-ProDXd as a white solid.

### LP1

### {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamate (LP1)

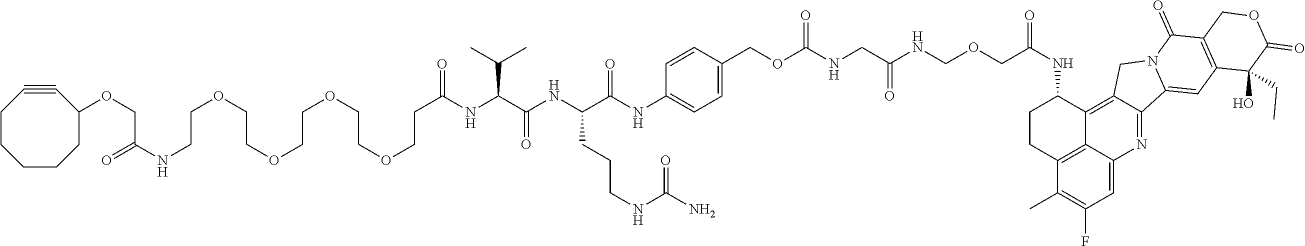 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

Following the general procedure starting from payload P1 (0.85 g, 1.2 mmol) catalyzed by HOBt, linker-payload LP1 (1.1 g, 62% yield) was obtained as a white solid after purification by prep-HPLC (5-60% acetonitrile in aq. formic acid (0.1%)). ESI m/z: 699.0 (M/2+H)+. ¹H NMR (400 MHz, DMSO_{d6}) δ 10.00 (s, 1H), 8.80 (t, J=6.4 Hz, 1H), 8.51 (d, J=8.8 Hz, 1H), 8.13 (d, J=7.6 Hz, 1H), 7.90 (d, J=8.8 Hz, 1H), 7.79 (d, J=10.8 Hz, 1H), 7.65-7.50 (m, 3H), 7.43 (t, J=6.0 Hz, 1H), 7.31 (s, 1H), 7.27 (d, J=8.8 Hz, 2H), 6.54 (s, 1H), 5.98 (t, J=5.2 Hz, 1H), 5.63-5.57 (m, 1H), 5.41 (s, 4H), 5.21 (s, 2H), 4.92 (s, 2H), 4.62 (d, J=6.4 Hz, 2H), 4.43-4.33 (m, 1H), 4.31-4.17 (m, 2H), 4.01 (s, 2H), 3.86 (d, J=14.4 Hz, 1H), 3.75 (d, J=14.8 Hz, 1H), 3.67-3.46 (m, 15H), 3.44-3.39 (m, 2H), 3.27-3.10 (m, 4H), 3.06-2.90 (m, 2H), 2.47-2.32 (m, 5H), 2.26-1.64 (m, 14H), 1.63-1.52 (m, 3H), 1.47-1.32 (m, 3H), 0.90-0.80 (m, 9H) ppm. ¹⁹F NMR (376 MHz, DMSO_{d6}) δ −111 ppm.

### LP2

### {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-[(1S)-1-{[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}-2-hydroxyethyl]carbamate (LP2)

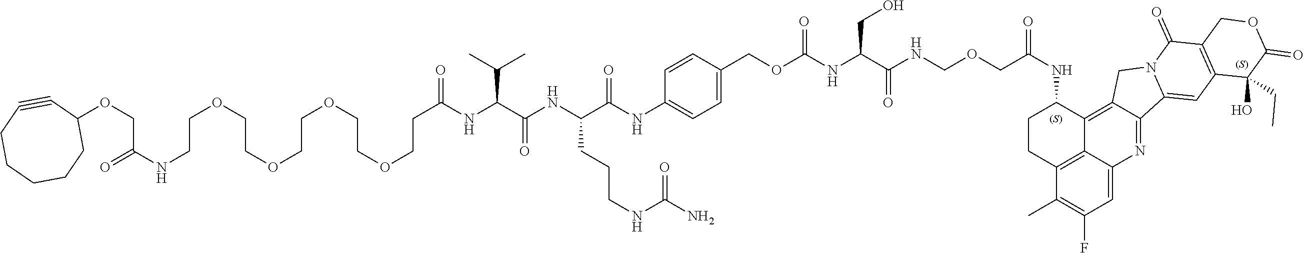

Following the general procedure starting from payload P2 (18 mg, 29 μmol) catalyzed by HOAt, linker-payload LP2 (19 mg, 45% yield) was obtained as a white solid after purification by prep-HPLC (5-95% acetonitrile in aq. TFA (0.1%)). ESI m/z: 714 (M/2+H)⁺. ¹H NMR (400 MHz, DMSO_{d6}) δ 10.00 (s, 1H), 8.82 (t, J=6.7 Hz, 1H), 8.50 (d, J=8.6 Hz, 1H), 8.14 (d, J=7.2 Hz, 1H), 7.89 (d, J=8.6 Hz, 1H), 7.79 (d, J=11.0 Hz, 1H), 7.66-7.54 (m, 3H), 7.35-7.24 (m, 3H), 7.18 (d, J=8.2 Hz, 1H), 6.59-6.46 (m, 1H), 5.99 (s, 1H), 5.65-5.55 (m, 1H), 5.42 (s, 3H), 5.21 (s, 2H), 4.97-4.83 (m, 2H), 4.67-4.57 (m, 2H), 4.41-4.34 (m, 1H), 4.30-4.21 (m, 2H), 4.00 (s, 2H), 3.99-3.65 (m, 3H), 3.62-3.57 (m, 2H), 3.53-3.46 (m, 12H), 3.28-3.16 (m, 4H), 3.06-2.81 (m, 3H), 2.39 (s, 3H), 2.26-2.02 (m, 6H), 2.02-1.80 (m, 6H), 1.78-1.66 (m, 3H), 1.62-1.52 (m, 3H), 1.48-1.22 (m, 8H), 0.92-0.77 (m, 9H) ppm.

### LP3

### {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-[(1S)-3-carbamoyl-1-{[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}propyl]carbamate (LP3)

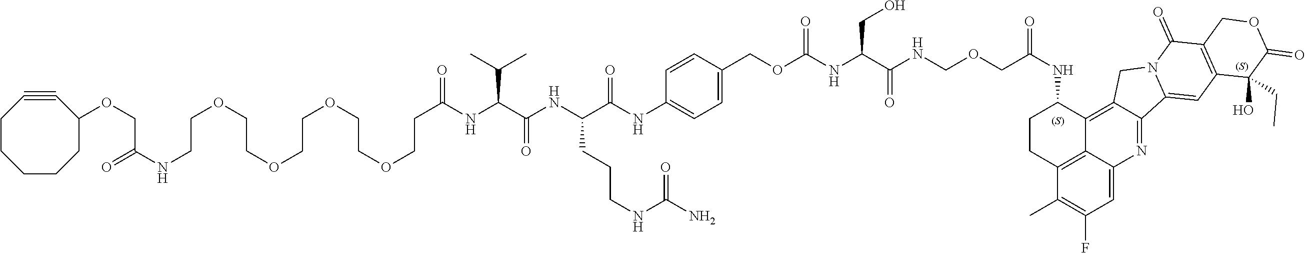

Following the general procedure starting from payload P3 (14 mg, 22 μmol) catalyzed by HOAt, linker-payload LP3 (16 mg, 49% yield) was obtained as a white solid after purification by prep-HPLC (5-95% acetonitrile in aq. TFA (0.1%)). ESI m/z: 734 (M/2+H)⁺. ¹H NMR (400 MHz, DMSO_{d6}) δ 10.00 (s, 1H), 8.83 (t, J=6.5 Hz, 1H), 8.54 (d, J=8.6 Hz, 1H), 8.14 (d, J=7.0 Hz, 1H), 7.89 (d, J=8.7 Hz, 1H), 7.79 (d, J=10.8 Hz, 1H), 7.66-7.51 (m, 3H), 7.44 (d, J=7.4 Hz, 1H), 7.31-7.23 (m, 3H), 6.78 (s, 1H), 6.59-6.48 (m, 1H), 6.03-5.94 (m, 1H), 5.64-5.56 (m, 1H), 5.42 (s, 3H), 5.22 (s, 2H), 4.95-4.81 (m, 2H), 4.68-4.56 (m, 2H), 4.42-4.34 (m, 1H), 4.31-4.19 (m, 2H), 4.01 (s, 2H), 3.87 (d, J=14.8 Hz, 2H), 3.75 (d, J=14.8 Hz, 1H), 3.64-3.55 (m, 2H), 3.53-3.44 (m, 12H), 3.28-3.20 (m, 4H), 3.08-2.90 (m, 3H), 2.40 (s, 3H), 2.24-2.02 (m, 8H), 2.01-1.50 (m, 16H), 1.46-1.29 (m, 4H), 0.90-0.76 (m, 9H) ppm.

### LP4

### (4S)-4-{[({4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methoxy)carbonyl]amino}-4-{[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}butanoic acid (LP4)

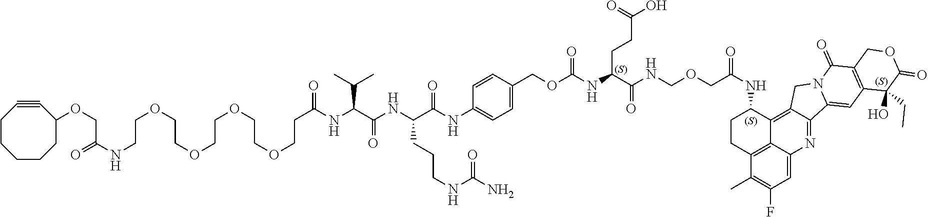

Following the general procedure starting from payload P4 (16 mg, 25 μmol) catalyzed by HOAt, linker-payload LP4 (12 mg, 35% yield) was obtained as a white solid after purification by prep-HPLC (5-95% acetonitrile in aq. TFA (0.1%)). ESI m/z: 735 (M/2+H)⁺. ¹H NMR (400 MHz, DMSO_{d6}) δ 9.99 (s, 1H), 8.89-8.82 (m, 1H), 8.53 (d, J=8.5 Hz, 1H), 8.12 (d, J=7.5 Hz, 1H), 7.88 (d, J=8.9 Hz, 1H), 7.78 (d, J=11.1 Hz, 1H), 7.65-7.51 (m, 3H), 7.44 (d, J=7.4 Hz, 1H), 7.33-7.22 (m, 2H), 6.53 (s, 1H), 6.04-5.95 (m, 1H), 5.64-5.54 (m, 1H), 5.42 (s, 3H), 5.21 (s, 2H), 4.97-4.81 (m, 2H), 4.67-4.56 (m, 2H), 4.41-4.33 (m, 1H), 4.31-4.18 (m, 2H), 4.00 (s, 2H), 3.99-3.65 (m, 3H), 3.63-3.55 (m, 2H), 3.54-3.42 (m, 12H), 3.27-3.17 (m, 4H), 3.09-2.87 (m, 3H), 2.39 (s, 3H), 2.27-1.13 (m, 28H), 0.98-0.64 (m, 9H) ppm.

### LP5

### {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-[(5S)-5-amino-5-{[({[(10S,23S)-10-ethyl-18-fluoro-10-hydroxy-19-methyl-5,9-dioxo-8-oxa-4,15-diazahexacyclo[14.7.1.0²,4.0^{4,13}.0^{6,11}.0^{20,24}]tetracosa-1,6(11),12,14,16,18,20(24)-heptaen-23-yl]carbamoyl}methoxy)methyl]carbamoyl}pentyl]carbamate (LP5)

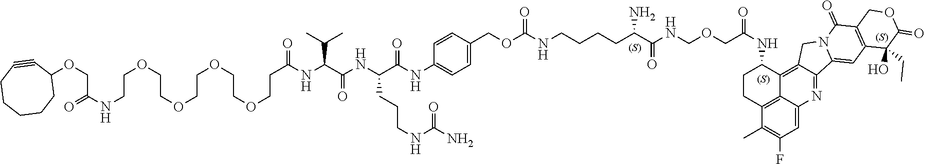

Following the general procedure starting from compound 5da (32 mg, 37 μmol) with intermediate D catalyzed by HOBt, Fmoc-LP5 (35 mg, 56% yield) was obtained as a white solid after purification by prep-HPLC (5-95% acetonitrile in aq. TFA (0.1%)). To a solution of Fmoc-LP5 (35 mg, 21 μmol) in DMF (2 mL) was added diethylamine (7.6 mg, 0.10 mmol), and the reaction mixture was stirred at room temperature for 2 hours until Fmoc was totally removed according to LCMS. The resulting mixture was directly purified by reversed phase flash chromatography (5-95% acetonitrile in aq. TFA (0.01%)) to give LP5 (9.6 mg, 31% yield) as a white solid. ESI m/z: 734 (M/2+H)⁺. ¹H NMR (400 MHz, DMSO_{d6}) δ 10.00 (d, J=13.5 Hz, 1H), 9.19-9.04 (m, 1H), 9.01-8.87 (m, 1H), 8.65-8.55 (m, 1H), 8.20-8.08 (m, 1H), 7.92-7.86 (m, 1H), 7.85-7.75 (m, 1H), 7.71 (s, 1H), 7.63-7.54 (m, 2H), 7.33-7.15 (m, 3H), 6.54 (s, 1H), 6.06-5.97 (m, 1H), 5.63-5.56 (m, 1H), 5.45-5.39 (m, 2H), 5.20 (s, 1H), 5.14-5.04 (m, 1H), 4.90 (d, J=6.1 Hz, 2H), 4.69-4.57 (m, 2H), 4.40-4.33 (m, 1H), 4.31-4.18 (m, 2H), 4.10-3.96 (m, 2H), 3.99-3.65 (m, 3H), 3.65-3.55 (m, 4H), 3.53-3.44 (m, 12H), 3.29-3.19 (m, 4H), 3.03-2.35 (m, 7H), 2.27-1.17 (m, 32H), 0.91-0.63 (m, 9H) ppm.

### LP5C

### {4-[(2S)-5-(carbamoylamino)-2-[(2S)-3-methyl-2-(1-{2-[4-(6-methyl-1,2,4,5-tetrazin-3-yl)phenyl]acetamido}-3,6,9,12-tetraoxapentadecan-15-amido)butanamido]pentanamido]phenyl}methyl N-[(5S)-5-amino-5-{[({[(10S,23S)-10-ethyl-18-fluoro-10-hydroxy-19-methyl-5,9-dioxo-8-oxa-4,15-diazahexacyclo[14.7.1.0²,4.0^{4,13}.0^{6,11}.0^{20,24}]tetracosa-1,6(11),12,14,16,18,20(24)-heptaen-23-yl]carbamoyl}methoxy)methyl]carbamoyl}pentyl]carbamate (LP5C)

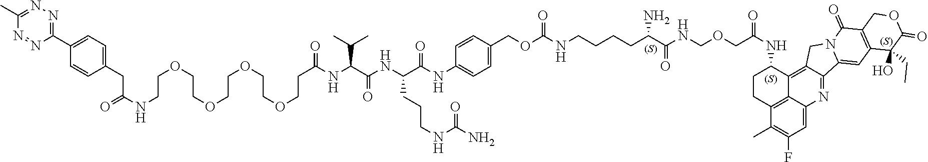

Following the similar procedure as LP5 except using intermediate Dd instead of intermediate Da, linker-payload LP5C (15 mg, 17% yield) was obtained as a red solid after purification by prep-HPLC (5-95% acetonitrile in aq. TFA (0.1%)). ESI m/z: 758 (M/2+H)⁺.

### LP7

### {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-[(1S)-1-{[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}-2-phenylethyl]carbamate (LP7)

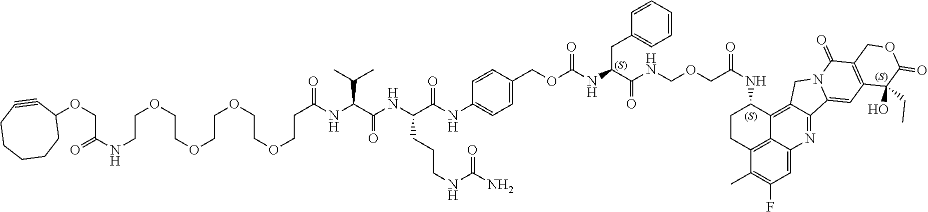

Following the general procedure starting from payload P7 (17 mg, 25 μmol) catalyzed by HOAt, linker-payload LP7 (17 mg, 46% yield) was obtained as a white solid after purification by prep-HPLC (5-95% acetonitrile in aq. TFA (0.1%)). ESI m/z: 744 (M/2+H)⁺. ¹H NMR (400 MHz, DMSO_{d6}) δ 9.98 (s, 1H), 8.97 (t, J=6.8 Hz, 1H), 8.54 (d, J=8.7 Hz, 1H), 8.13 (d, J=7.2 Hz, 1H), 7.89 (d, J=8.8 Hz, 1H), 7.79 (d, J=11.0 Hz, 1H), 7.62 (t, J=5.5 Hz, 1H), 7.55 (d, J=8.5 Hz, 2H), 7.49 (d, J=8.2 Hz, 1H), 7.30 (s, 1H), 7.26-7.20 (m, 4H), 7.18-7.12 (m, 2H), 6.03-5.95 (m, 1H), 5.64-5.56 (m, 1H), 5.47-5.34 (m, 3H), 5.26-5.14 (m, 2H), 4.86-4.75 (m, 2H), 4.70-4.57 (m, 2H), 4.41-4.33 (m, 1H), 4.31-4.12 (m, 4H), 3.99 (s, 2H), 3.99-3.65 (m, 3H), 3.63-3.57 (m, 3H), 3.50-3.47 (m, 12H), 3.28-3.10 (m, 4H), 2.98-2.90 (m, 1H), 2.39 (s, 3H), 2.28-1.16 (m, 25H), 0.87-0.80 (m, 9H) ppm.

### LP8

### {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-{[({[({[(10S,23S)-10-ethyl-18-fluoro-10-hydroxy-19-methyl-5,9-dioxo-8-oxa-4,15-diazahexacyclo[14.7.1.0²,4.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamoyl]methyl}carbamate (LP8)

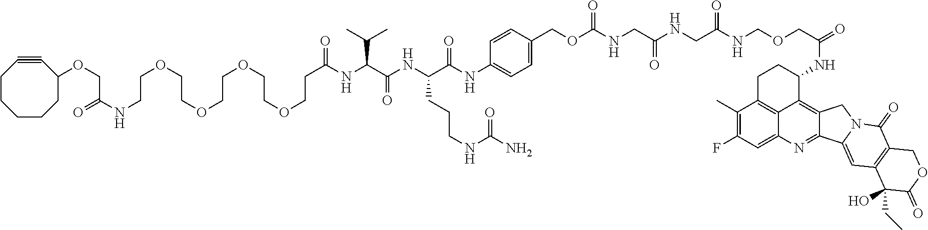 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

Following the general procedure starting from payload P8 (20 mg, 32 μmol) catalyzed by HOBt, linker-payload LP8 (14 mg, 29% yield) was obtained as a white solid after purification by prep-HPLC (5-95% acetonitrile in aq. TFA (0.1%)). ESI m/z: 727 (M/2+H)⁺. ¹H NMR (400 MHz, DMSO_{d6}) δ 9.99 (s, 1H), 8.72-8.68 (m, 1H), 8.51 (d, J=8.5 Hz, 1H), 8.19-8.12 (m, 2H), 7.88 (d, J=9.1 Hz, 1H), 7.80 (d, J=11.2 Hz, 1H), 7.63-7.55 (m, 3H), 7.47-7.43 (m, 1H), 7.31 (s, 1H), 7.28 (d, J=8.3 Hz, 2H), 6.53 (s, 1H), 6.00-5.95 (m, 1H), 5.62-5.58 (m, 1H), 5.42 (d, J=4.9 Hz, 3H), 5.21 (s, 2H), 4.94 (s, 2H), 4.63 (d, J=6.4 Hz, 2H), 4.39-4.35 (m, 1H), 4.28-4.22 (m, 2H), 4.01 (s, 2H), 3.85 (s, 1H), 3.77 (s, 1H), 3.74-3.70 (m, 2H), 3.66-3.57 (m, 4H), 3.52-3.39 (m, 14H), 3.28-3.21 (m, 4H), 3.04-2.97 (m, 2H), 2.40 (s, 3H), 2.25-2.13 (m, 6H), 2.01-1.82 (m, 9H), 1.61-1.55 (m, 3H), 1.49-1.36 (m, 5H), 0.91-0.78 (m, 9H) ppm.

### LP9

### {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl](methyl)carbamoyl}methyl)carbamate (LP9)

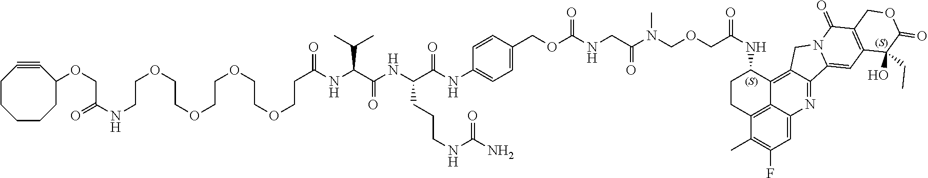

Following the general procedure starting from payload P9 (6.0 mg, 10 μmol) catalyzed by HOBt, linker-payload LP9 (5.0 mg, 36% yield) was obtained as a white solid after purification by prep-HPLC (5-95% acetonitrile in aq. TFA (0.1%)). ESI m/z: 706 (M/2+H)⁺. ¹H NMR (400 MHz, DMSO_{d6}) δ 9.99 (s, 1H), 8.63-8.50 (m, 1H), 8.15-8.08 (m, 1H), 7.88-7.78 (m, 2H), 7.59-7.57 (m, 3H), 7.30-7.22 (m, 4H), 6.52 (s, 1H), 5.98 (s, 1H), 5.61 (s, 1H), 5.42 (br, 4H), 5.22 (s, 2H), 4.88-4.82 (m, 4H), 4.42-4.20 (m, 4H), 4.09-3.74 (m, 8H), 3.59-3.49 (m, 13H), 3.25-3.23 (m, 4H), 3.00-2.88 (m, 6H), 2.39-2.38 (m, 4H), 2.20-2.16 (m, 3H), 1.97-1.73 (m, 9H), 1.56-1.35 (m, 7H), 0.86-0.83 (m, 9H) ppm.

### Example 12. Exemplary Synthesis of Linker-payloads by route 3b (Scheme 4D)

### LP1 synthesized from Fmoc-P1 (5a) reacted with intermediate D using HOBt {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamate (LP1)

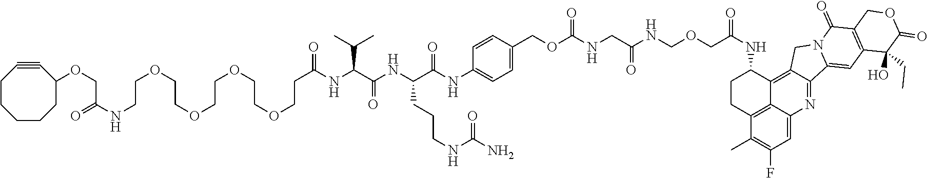 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

To a yellow solution of 5a (1.7 g, 2 mmol) in DMF (17 mL) were added DBU (30 mg, 0.20 mmol) and triethylamine (0.40 g, 4.0 mmol) at 25° C., and the mixture was stirred at 25° C. for 15 minutes. To the reaction mixture were added HOBt (0.14 g, 1.0 mmol) and intermediate D (2.0 g, 2.1 mmol), and the resulting clear solution was stirred at 25° C. for 16 hours. The resulting mixture was poured into MTBE (150 mL) and the heterogeneous mixture was stirred at room temperature for 5 minutes. The MTBE layer, containing most of Fmoc-ene side-product and bases, was then separated off. The black oil in the bottom was diluted with DMF (20 mL) and the solution was purified by prep-HPLC (5-95% acetonitrile in aq. TFA (0.01%)) to give LP1 (1.5 g, 55% yield) as a white solid. ESI m/z: 1396 (M+H)⁺. ¹H NMR (400 MHz, DMSO_{d6}) δ 10.00 (t, J=6.4 Hz, 1H), 8.80 (t, J=6.4 Hz, 1H), 8.51 (d, J=8.8 Hz, 1H), 8.13 (d, J=7.6 Hz, 1H), 7.90 (d, J=8.8 Hz, 1H), 7.79 (d, J=10.8 Hz, 1H), 7.65-5.50 (m, 3H), 7.43 (t, J=6.0 Hz, 1H), 7.31 (s, 1H), 7.27 (d, J=8.8 Hz, 2H), 6.54 (s, 1H), 5.98 (t, J=5.2 Hz, 1H), 5.63-5.57 (m, 1H), 5.41 (s, 4H), 5.21 (s, 2H), 4.92 (s, 2H), 4.62 (d, J=6.4 Hz, 2H), 4.43-4.33 (m, 1H), 4.31-4.17 (m, 2H), 4.01 (s, 2H), 3.86 (d, J=14.4 Hz, 1H), 3.75 (d, J=14.8 Hz, 1H), 3.67-3.54 (m, 4H), 3.53-3.46 (m, 12H), 3.44-3.39 (m, 2H), 3.27-3.10 (m, 4H), 3.06-2.90 (m, 2H), 2.47-2.32 (m, 5H), 2.26-1.64 (m, 14H), 1.63-1.52 (m, 3H), 1.47-1.32 (m, 3H), 0.90-0.80 (m, 9H) ppm. ¹⁹F NMR (376 MHz, DMSO_{d6}) δ −111 (Ar-F) ppm.

### LP1 synthesized from Fmoc-P1 (5a) reacted with intermediate D using MeHYQ (small scale)

### {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamate (LP1)

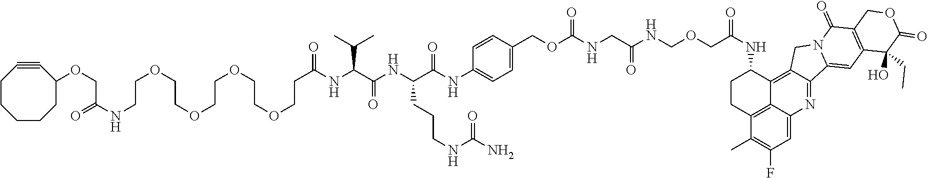 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

To a yellow solution of 5a (10 mg, 1 eq.), intermediate D (12.5 mg, 1.05 eq.) and 4-methyl-2-hydroxyquinoline (MeHYQ) (1.0 mg, 0.5 mmol) in DMF (130 uL) were added DBU (0.24 mg, 1.6 umol) and triethylamine (3.2 mg, 32 umol) at 25° C. The clear solution was stirred at 50° C. for 1.5 hours, which was monitored by LCMS. After cooled to room temperature, the resulting mixture was poured into stirred MTBE (600 uL) at 0-10° C. and brown oil appeared, which was collected after separation to remove the MTBE layer. The oil was then purified by reversed phase flash chromatography (5-95% acetonitrile in aq. TFA (0.01%)) to give LP1 (10 mg, 90% yield) as a white solid. ESI m/z: 699.0 (M/2+H)⁺.

### LP1 synthesized from Fmoc-P1 (5a) reacted with intermediate D using MeHYQ (large scale)

### {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamate (LP1)

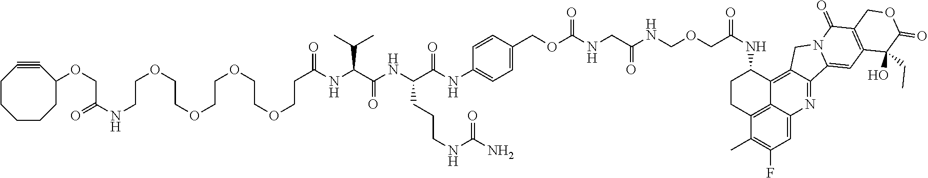 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

To a yellow solution of Fmoc-ProDXd (5a) (13 g, 16 mmol), intermediate Da (16 g, 17 mmol), and 4-methyl-2-hydroxyquinoline (MeHYQ) (1.3 g, 8.2 mmol) in DMF (130 mL) were added DBU (0.24 g, 1.6 mmol) and triethylamine (3.2 g, 32 mmol) at 25° C. The clear solution was stirred at 50° C. for 6 hours, which was monitored by LCMS. After cooled to room temperature, the resulting mixture was poured into stirred MTBE (600 mL) at 0-10° C. and brown oil appeared, which was collected after separation to remove the MTBE layer. The oil was then purified by reversed phase flash chromatography (5-95% acetonitrile in aq. TFA (0.01%)) to give LP1 (13 g, 58% yield) as a white solid. ESI m/z: 698.8 (M/2+H)⁺.

¹H NMR (400 MHz, DMSO_{d6}) δ 10.00 (t, J=6.4 Hz, 1H), 8.80 (t, J=6.4 Hz, 1H), 8.51 (d, J=8.8 Hz, 1H), 8.13 (d, J=7.6 Hz, 1H), 7.90 (d, J=8.8 Hz, 1H), 7.79 (d, J=10.8 Hz, 1H), 7.65-5.50 (m, 3H), 7.43 (t, J=6.0 Hz, 1H), 7.31 (s, 1H), 7.27 (d, J=8.8 Hz, 2H), 6.54 (s, 1H), 5.98 (t, J=5.2 Hz, 1H), 5.63-5.57 (m, 1H), 5.41 (s, 4H), 5.21 (s, 2H), 4.92 (s, 2H), 4.62 (d, J=6.4 Hz, 2H), 4.43-4.33 (m, 1H), 4.31-4.17 (m, 2H), 4.01 (s, 2H), 3.86 (d, J=14.4 Hz, 1H), 3.75 (d, J=14.8 Hz, 1H), 3.67-3.54 (m, 4H), 3.53-3.46 (m, 12H), 3.44-3.39 (m, 2H), 3.27-3.10 (m, 4H), 3.06-2.90 (m, 2H), 2.47-2.32 (m, 5H), 2.26-1.64 (m, 14H), 1.63-1.52 (m, 3H), 1.47-1.32 (m, 3H), 0.90-0.80 (m, 9H) ppm. ¹⁹F NMR (376 MHz, DMSO_{d6}) δ −111 (Ar-F) ppm.

¹³C NMR (100 MHz, DMSO_{d6}) δ 172.36, 171.11, 170.65, 170.61, 170.31, 169.20, 168.68, 162.70, 160.23, 158.90, 156.58, 156.43, 152.14, 149.89, 147.79, 147.65, 144.99, 140.48, 138.59, 136.24, 131.52, 128.56, 125.08, 123.65, 123.45, 121.50, 118.83, 109.81, 109.58, 100.73, 96.67, 92.06, 72.30, 72.10, 69.74, 69.48, 68.76, 67.80, 66.87, 65.31, 57.48, 53.07, 49.54, 44.63, 43.53, 41.62, 35.88, 33.83, 30.54, 30.32, 29.19, 27.80, 26.77, 25.80, 23.72, 19.96, 19.13, 18.05, 10.86, 7.69 ppm.

| | Reactant | | | | | | | | | | | | | | | | |
|---|---|---|---|---|---|---|---|---|---|---|---|---|---|---|---|---|---|
| | Fmoc- | | | | Reagents | | | | | | Sol | Temp. | time | puri- | Product LP1 | | |
| # | ProDXd | | Int D | | Et3N | | DBU | | MeHYQ | | DMF | (° C.) | (h) | fication | Amt | Yield | Purity |
| 1 | 100 | mg | 125 | mg | 24 | mg | 1.8 | mg | 9.9 | mg | 1 ml | 50 | 4 | C18 | 80 mg | 46% | 96.9% |
| | 0.12 | mmol | 0.13 | mmol | 0.24 | mmol | 0.012 | mmol | 0.06 | mmol | | | | column | | | in |
| | | | (1.05 eq.) | | (2.0 eq.) | | (0.1 eq.) | | (0.5 eq.) | | | | | | | | HPLC |
| 2 | 1.0 | g | 1.2 | g | 240 | mg | 18 | mg | 99 | mg | 10 ml | 50 | 5 | C18 | 1.0 g | 57% | 96.9% |
| | 1.2 | mmol | 1.3 | mmol | 2.4 | mmol | 0.12 | mmol | 0.60 | mmol | | | | column | | | in |
| | | | (1.05 eq.) | | (2.0 eq.) | | (0.1 eq.) | | (0.5 eq.) | | | | | | | | HPLC |
| 3 | 13 | g | 16 | g | 3.23 | g | 243 | mg | 1.27 | g | 13 ml | 50 | 6 | C18 | 13 g | 58% | 99.9% |
| | 16 | mmol | 16.8 | mmol | 32 | mmol | 1.6 | mmol | 8 | mmol | | | | column | | | in |

| | | | | |
|---|---|---|---|---|
| (1.05 eq.) | (2.0 eq.) | (0.1 eq.) | (0.5 eq.) | HPLC |

Certificate of Analysis of LP1 (1g lot):

### Chemical Structure:

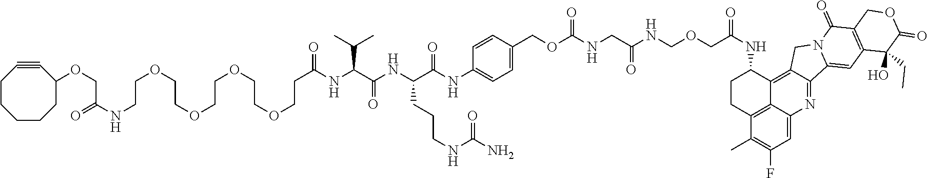 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

| Compound information | Results |
|---|---|
| Mol. Formula | C₆₉H₉₀FN₁₁O₁₉ |
| MW (with salt, solvent.) | 1396.54 |
| Salt/active Substance ratio | N/A |
| Appearance | white solid |
| LC-MS(exact mass) | [M + H]⁺ = 1396.7 |
| HPLC Purity | 99.99% |
| Chiral HPLC Purity | 99ee % |

Certificate of Analysis of LP1 (13g lot)

### Chemical Structure:

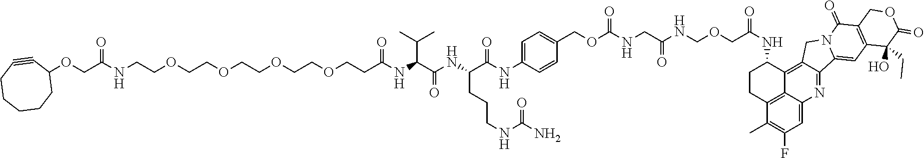 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

| Physical and | |
|---|---|
| Chemical Properties | Results |
| Salt | Free base |
| Mol. Formula | C₆₉H₉₀FN₁₁O₁₉ |
| Molecular Weight | 1396.54 |
| Storage | Store solid at −20° C. |
| Appearance | white solid at −20° C., but turns to be solid-oil |
| | at room temperature |
| Solubility | soluble in 10 mM DMSO and 26 uM water |
| Purity | >98% by HPLC and LC-MS |
| Special liability | Light sensitive, unstable at pH <3 or pH >11 |
| Absorbance | at 254 nm and 214 nm |
| Payload class | Topoisomerase I inhibitor |
| Hydrophobicity range | CLogP = 0.72 |

Analytical tests and results

| Item | Criterion |
|---|---|
| HPLC Purity | >98% |
| Chiral HPLC Purity | ee > 98% |

### Example 13. Exemplary 2-step-synthesis of Linker-payloads by route 4 (Scheme 4E)

### LP1 synthesized from P1 with Fmoc-vcPAB and then reacted with intermediate Ba {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamate (LP1)

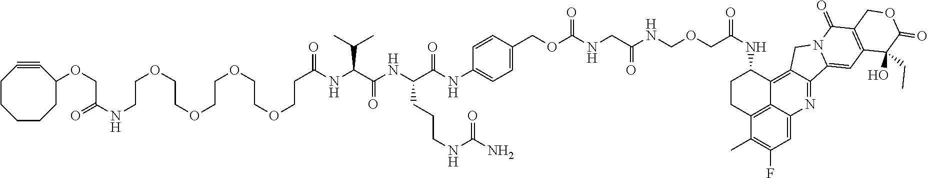 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

### [Step 1] vcPAB-P1

To a solution of compound Fmoc-vcPAB-PNP (0.36 g, 0.47 mmol, 1.0 eq., commercial) in DMF (2 mL) was added P1 (0.27 g, 0.47 mmol, 1.0 eq.), HOAt (95 mg, 0.70 mmol, 1.5 eq.) and DIPEA (0.12 mg, 0.94 mmol, 2.0 eq.), and the reaction mixture was stirred at room temperature for 4 hours, which was monitored by LCMS. The resulting mixture was directly purified by reversed phase flash chromatography to give the compound Fmoc-vcPAB-P1 (0.22 mg, ESI m/z: 1207 (M+H)⁺) as a yellow solid, which was dissolved in DMF (2 mL). To the solution was added diethylamine (0.2 mL), and the reaction mixture was stirred at room temperature for 2 hours, which was monitored by LCMS. The resulting mixture was directly purified by reversed phase flash chromatography to give vcPAB-P1 (0.16 g, 28% yield from P1) as a white solid. ESI m/z: 1085 (M+H)⁺.

### [Step 2]: LP1

To a solution of COT-PEG4-acid (intermediate Ba) (63 mg, 0.15 mmol, 1.0 eq., synthesized according to WO2018089373) in DMF (2 mL) were added HATU (83 mg, 0.22 mmol, 1.5 eq.) and DIPEA (58 mg, 0.45 mmol, 3.0 eq.), and the reaction mixture was stirred at room temperature for an hour before the addition of vcPAB-P1 (0.16 g, 0.15 mmol, 1.0 eq.). The reaction mixture was stirred at room temperature for 4 hours, which was monitored by LCMS. The resulting mixture was directly purified by prep-HPLC to give LP1 (20 mg, 10% yield) as a white solid. ESI m/z: 1396 (M+H)⁺.

LP1A synthesized from P1 with Fmoc-vcPAB and then reacted with intermediate Bb {4-[(2S)-5-(carbamoylamino)-2-[(2S)-2-[1-({[(4E)-cyclooct-4-en-1-yloxy]carbonyl}amino)-3,6,9,12-tetraoxapentadecan-15-amido]-3-methylbutanamido]pentanamido]phenyl}methyl N-({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamate (LP1A)

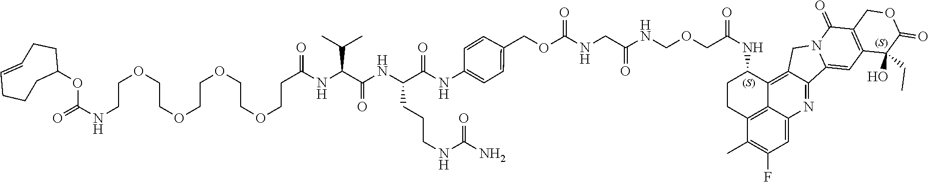

Following the similar procedure as LP1 except using TCO-PEG4-acid (Bb) instead of Ba, linker-payload LP1A (9.7 mg, 35% yield) was obtained as a white solid. ESI m/z: 692.8 (M/2+H)⁺. ¹H NMR (400 MHz, DMSO) δ 10.02 (s, 1H), 8.80 (s, 1H), 8.56-8.45 (m, 2H), 8.14 (d, J=7.0 Hz, 1H), 7.89 (d, J=8.7 Hz, 1H), 7.79 (d, J=10.7 Hz, 1H), 7.59 (d, J=8.3 Hz, 2H), 7.43 (s, 1H), 7.35-7.21 (m, 3H), 6.94 (s, 1H), 6.53 (s, 1H), 6.01 (s, 1H), 5.60 (s, 2H), 5.44 (m, 5H), 5.20 (s, 2H), 4.92 (s, 2H), 4.62 (d, J=6.6 Hz, 2H), 4.37 (s, 1H), 4.27-4.17 (m, 2H), 4.01 (s, 2H), 3.65-3.30 (m, 17H), 3.36 (s, 2H), 3.05-2.97 (m, 4H), 2.45-2.34 (m, 5H), 2.25 (s, 3H), 2.20-1.40 (m, 16H), 0.90-0.80 (m, 9H) ppm. ¹⁹F NMR (376 MHz, DMSO_{d6}) δ −74, −111 ppm.

### LP1B synthesized from P1 with Fmoc-vcPAB and then reacted with intermediate Bc {4-[(2S)-5-(carbamoylamino)-2-[(2S)-2-[1-({[(4E)-cyclooct-4-en-1-yloxy]carbonyl}amino)-3,6,9,12-tetraoxapentadecan-15-amido]-3-methylbutanamido]pentanamido]phenyl}methyl N-({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamate (LP1B)

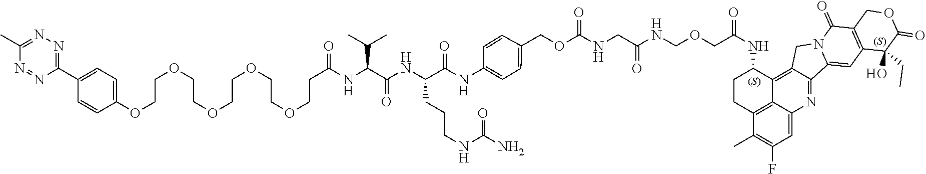

Following the similar procedure as LP1 except using MeTz-PEG4-acid (Bc) instead of Ba, linker-payload LP1B (20 mg, 55% yield) was obtained as a red solid. ESI m/z: 702.3 (M/2+H)⁺. ¹H NMR (400 MHz, DMSO_{d6}) δ 9.98 (s, 1H), 8.80 (d, J=6.6 Hz, 1H), 8.50 (d, J=9.0 Hz, 1H), 8.40 (d, J=8.9 Hz, 2H), 8.11 (d, J=7.5 Hz, 1H), 7.87 (d, J=8.7 Hz, 1H), 7.78 (d, J=11.1 Hz, 1H), 7.58 (d, J=8.5 Hz, 2H), 7.42 (t, J=5.7 Hz, 1H), 7.33-7.24 (m, 3H), 7.21 (t, J=7.1 Hz, 2H), 5.99 (br s, 1H), 5.60 (br s, 1H), 5.46-5.29 (m, 2H), 5.19 (s, 2H), 4.92 (s, 2H), 4.62 (d, J=6.4 Hz, 2H), 4.38 (d, J=5.1 Hz, 1H), 4.29-4.19 (m, 4H), 4.01 (br s, 2H), 3.83-3.76 (m, 3H), 3.63-3.54 (m, 6H), 3.52-3.46 (m, 8H), 3.25-3.13 (m, 2H), 3.08-2.80 (m, 5H), 2.41-2.33 (m, 5H), 2.18 (s, 2H), 1.91-1.71 (m, 4H), 1.64-1.50 (m, 3H), 1.50-1.20 (m, 4H), 0.88-0.80 (m, 9H) ppm. ¹⁹F NMR (376 MHz, DMSO_{d6}) δ −74, −111 ppm.

### Example 14. Exemplary Synthesis of Linker-payloads by route 5 (Scheme 4F)

### LP1 synthesized from PEG4-vcPAB-P1 with intermediate A {4-[(2S)-2-[(2S)-2-(1-amino-3,6,9,12-tetraoxapentadecan-15-amido)-3-methylbutanamido]-5-(carbamoylamino)pentanamido]phenyl}methyl N-({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamate (PEG4-vcPAB-P1)

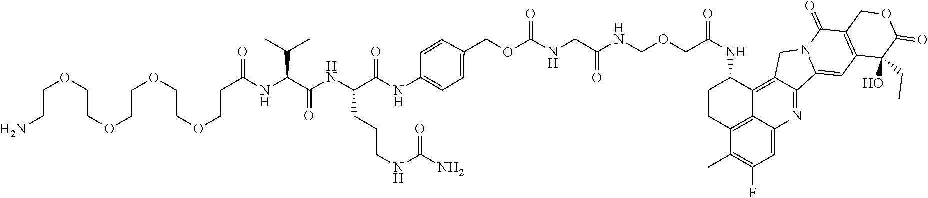 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCN)C(C)C)cc1)CC3]

To a solution of intermediate D-3 (85 mg, 0.10 mmol) in DMF (2 mL) were added DMAP (12 mg, 0.10 mmol), DIPEA (39 mg, 0.30 mmol) and bis(4-nitrophenyl) carbonate (91 mg, 0.30 mmol), and the reaction mixture was stirred at room temperature for an hour, which was monitored by LCMS. The resulting mixture was purified by reversed phase flash chromatography (0-60% acetonitrile in water) to give D-3-PNP as oil, which was dissolved in DMF (2 mL). To the solution were then added HOBt (6.8 mg, 50 μmol), DIPEA (39 mg, 0.30 mmol) and P1 (69 mg, 0.10 mmol, TFA salt), and the reaction mixture was stirred at room temperature for 2 hours, which was monitored by LCMS. The resulting mixture was directly purified by reversed phase flash chromatography (0-100% acetonitrile in aq. TFA (0.1%)) to give Fmoc-PEG4-vcPAB-P1 as a white solid, which was dissolved in DMF (1 mL). To the solution was added diethylamine (0.1 mL), and the mixture was stirred at room temperature for an hour until Fmoc was totally removed, which was monitored by LCMS. The resulting mixture was directly purified by reversed phase flash chromatography (0-100% acetonitrile in aq. TFA (0.01%)) to give PEG4-vcPAB-P1 (72 mg, 53% yield, TFA salt) as a light yellow solid. ESI m/z: 616.9 (M/2+H)⁺.

### {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamate (LP1)

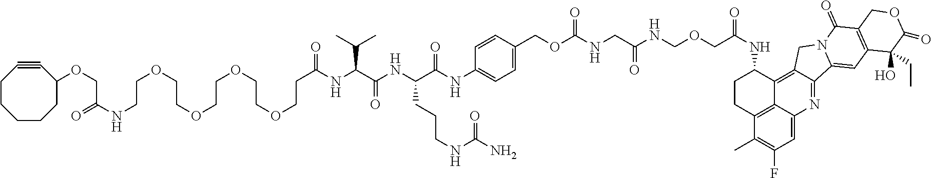 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

To a solution of PEG4-vcPAB-P1 (67 mg, 50 μmol) in DMF (1 mL) were added intermediate A (17 mg, 60 μmol) and DIPEA (19 mg, 0.15 mmol), and the reaction mixture was stirred at room temperature for an hour, which was monitored by LCMS. The reaction mixture was directly purified by prep-HPLC (5-95% acetonitrile in aq. TFA (0.1%)) to give LP1 (18 mg, 26% yield) as a white solid. ESI m/z: 699.0 (M/2+H)⁺.

### Example 15. Exemplary Synthesis of EvcPAB-Linker-payloads (Scheme 5A)

### LP5D

### (4S)-4-(1-amino-3,6,9,12-tetraoxapentadecan-15-amido)-4-{[(1S)-1-{[(1S)-4-(carbamoylamino)-1-{[4-({[(4-nitrophenoxy)carbonyl]oxy}methyl)phenyl]carbamoyl}butyl]carbamoyl}-2-methylpropyl]carbamoyl}butanoic acid (G-4)

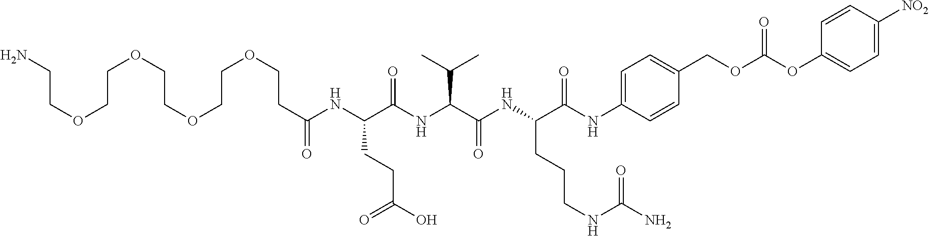 [SMILES: CC(C)[C@H](NC(=O)[C@H](CCC(=O)O)NC(=O)CCOCCOCCOCCOCCN)C(=O)N[C@@H](CCCNC(N)=O)C(=O)Nc1ccc(COC(=O)Oc2ccc([N+](=O)[O-])cc2)cc1]

To a solution of compound G-1 (0.30 g, 0.38 mmol) in dry DMF (4 mL) was added diethylamine (56 mg, 0.76 mmol), and the mixture was stirred at room temperature for 2 hours until Fmoc was totally removed, which was monitored by LCMS. The reaction mixture was purified by prep-HPLC to give a white solid (0.13 g, ESI m/z: 587.3 (M+Na)+), which was dissolved in dry DMF (3 mL). To the solution were added N-Boc-PEG4-acid (70 mg, 0.19 mmol), HATU (87 mg, 0.23 mmol) and DIPEA (99 mg, 0.23 mmol). The reaction mixture was stirred at room temperature for 2 hours, which was monitored by LCMS. The mixture was directly purified by prep-HPLC to give G-2 (0.15 g, 43% yield) as a white solid. ESI m/z: 913.3 (M+H)⁺.

To a solution of G-2 (0.14 g, 0.15 mmol) in DMF (1.5 mL) were added DIPEA (50 mg, 0.38 mmol) and bis(4-nitrophenyl) carbonate (70 mg, 0.23 mmol), and the reaction mixture was stirred at room temperature for 2 hours, which was monitored by LCMS. The mixture was separated by reversed phase flash chromatography (0-100% acetonitrile in aq. TFA (0.01%)) to give compound G-3 (0.19 g, 93% yield) as a white solid. ESI m/z: 1100 (M+Na)⁺.

To a solution of compound G-3 (0.18 g, 0.17 mmol) in acetonitrile (2.0 mL) was added HCl in ethyl acetate (4 M, 2 mL). The reaction mixture was stirred at room temperature for 3 hours. The mixture was purified by reversed phase flash chromatography (0-100% acetonitrile in aq. TFA (0.01%)) to give compound G-4 (0.10 g, 58% yield) as a white solid. ESI m/z: 943.5 (M+Na)⁺.

### (4S)-4-{[(1S)-1-{[(1S)-4-(carbamoylamino)-1-{[4-({[(4-nitrophenoxy)carbonyl]oxy}methyl)phenyl]carbamoyl}butyl]carbamoyl}-2-methylpropyl]carbamoyl}-4-{1-[2-(cyclooct-2-yn-1-yloxy)acetamido]-3,6,9,12-tetraoxapentadecan-15-amido}butanoic acid (Ga)

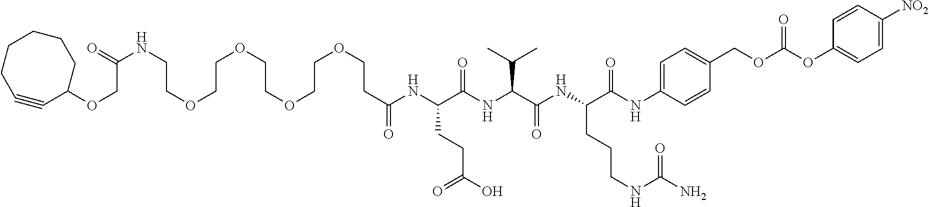 [SMILES: CC(C)[C@H](NC(=O)[C@H](CCC(=O)O)NC(=O)CCOCCOCCOCCOCCNC(=O)COC1C#CCCCCC1)C(=O)N[C@@H](CCCNC(N)=O)C(=O)Nc1ccc(COC(=O)Oc2ccc([N+](=O)[O-])cc2)cc1]

To a mixture of intermediate Aa (39 mg, 0.10 mmol) in DMF (1 mL) were added DIPEA (28 mg, 0.22 mmol) and a solution of compound G-4 (90 mg, 87 μmol) in DMF (1.5 mL). The reaction mixture was stirred at room temperature for an hour, which was monitored by LCMS. After the reaction completed, the mixture was immediately separated by prep-HPLC to give compound Ga (30 mg, 29% yield) as a white solid. ESI m/z: 1085 (M+H)+, 1107 (M+Na)⁺.

### (4S)-4-{[(1S)-1-{[(1S)-1-({4-[({[(5S)-5-amino-5-{[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}pentyl]carbamoyl}oxy)methyl]phenyl}carbamoyl)-4-(carbamoylamino)butyl]carbamoyl}-2-methylpropyl]carbamoyl}-4-{1-[2-(cyclooct-2-yn-1-yloxy)acetamido]-3,6,9,12-tetraoxapentadecan-15-amido}butanoic acid (LP5D)

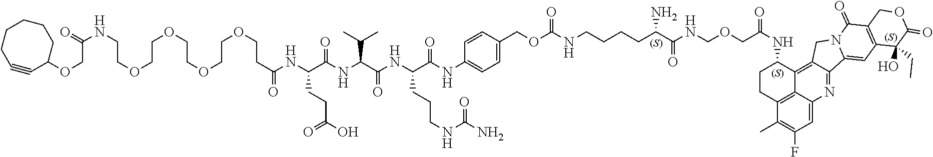

Following the similar procedure as LP5 in Example 7 except using compound Ga instead of intermediate D, linker-payload LP5D (7 mg, 14% yield) was obtained as a light yellow solid. ESI m/z: 799 (M/2+H). ¹H NMR (400 MHz, DMSO_{d6}) δ 12.09 (br s, 1H), 10.02 (s, 1H), 9.29 (br s, 1H), 8.59 (d, J=8.4 Hz, 1H), 8.25-7.99 (m, 5H), 7.80 (d, J=10.9 Hz, 1H), 7.72 (d, J=8.9 Hz, 1H), 7.62-7.58 (m, 3H), 7.34 (s, 1H), 7.31-7.10 (m, 3H), 6.55 (s, 1H), 5.98 (m, 1H), 5.61 (br s, 1H), 5.42 (br s, 4H), 5.21 (s, 2H), 4.91 (s, 2H), 4.81-4.64 (m, 2H), 4.38-4.14 (m, 4H), 4.06 (s, 2H), 3.82-3.77 (m, 2H), 3.62-3.58 (m, 2H), 3.53-3.44 (m, 12H), 3.41 (m, 2H), 3.28-3.14 (m, 4H), 3.05-2.96 (m, 4H), 2.44-2.29 (m, 6H), 2.28-2.13 (m, 6H), 2.07 (br s, 1H), 1.98-1.83 (m, 5H), 1.80-1.65 (m, 6H), 1.61-1.53 (m, 3H), 1.44-1.37 (m, 5H), 1.32-1.22 (m, 3H), 0.91-0.77 (m, 9H) ppm. ¹⁹F NMR (376 MHz, DMSO_{d6}) δ −73, −111 ppm.

### Example 16. Synthesis of Branched-linker-payloads

The synthesis of branch linker L13aE and L15aE were reported in WO2022015656. Compounds L13bE and L15bE were synthesized as described below.

| | | | |
|---|---|---|---|
| L13aE | 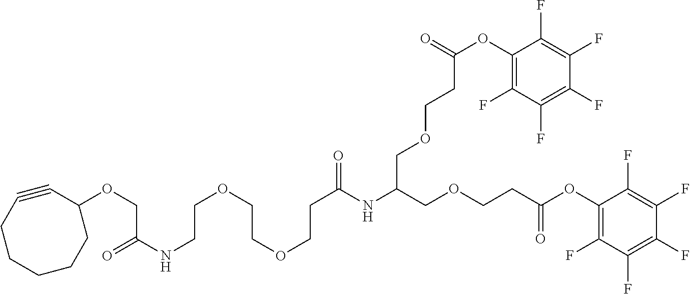 [SMILES: O=C(COC1C#CCCCCC1)NCCOCCOCCC(=O)NC(COCCC(=O)Oc1c(F)c(F)c(F)c(F)c1F)COCCC(=O)Oc1c(F)c(F)c(F)c(F)c1F] | 2758875- 40-8 | WO2022015656 |
| L13bE | 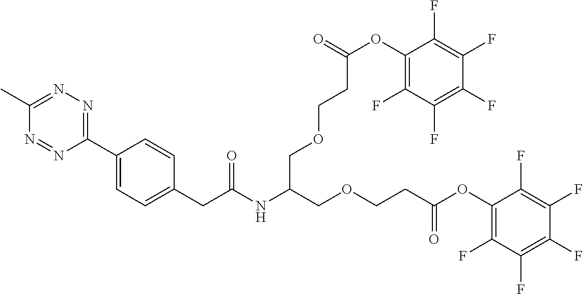 [SMILES: Cc1nnc(-c2ccc(CC(=O)NC(COCCC(=O)Oc3c(F)c(F)c(F)c(F)c3F)COCCC(=O)Oc3c(F)c(F)c(F)c(F)c3F)cc2)nn1] | | |
| L15aE | 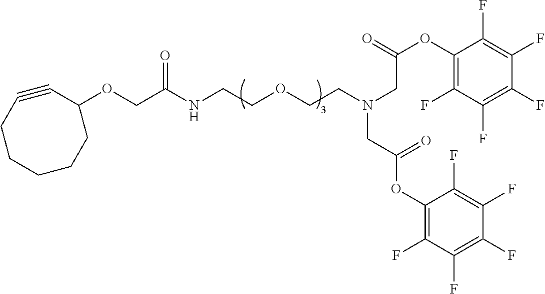 | 2758875- 37-3 | WO2022015656 |
| L15bE | 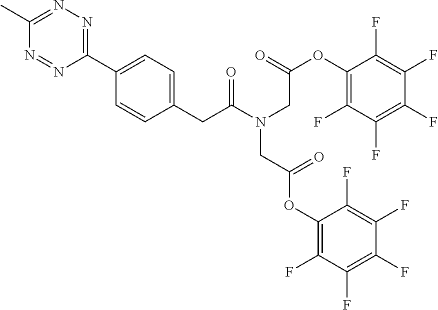 [SMILES: Cc1nnc(-c2ccc(CC(=O)N(CC(=O)Oc3c(F)c(F)c(F)c(F)c3F)CC(=O)Oc3c(F)c(F)c(F)c(F)c3F)cc2)nn1] | | |

### 2,3,4,5,6-pentafluorophenyl 3-(2-{2-[4-(6-methyl-1,2,4,5-tetrazin-3-yl)phenyl]acetamido}-3-[3-oxo-3-(2,3,4,5,6-pentafluorophenoxy)propoxy]propoxy)propanoate (L13bE)

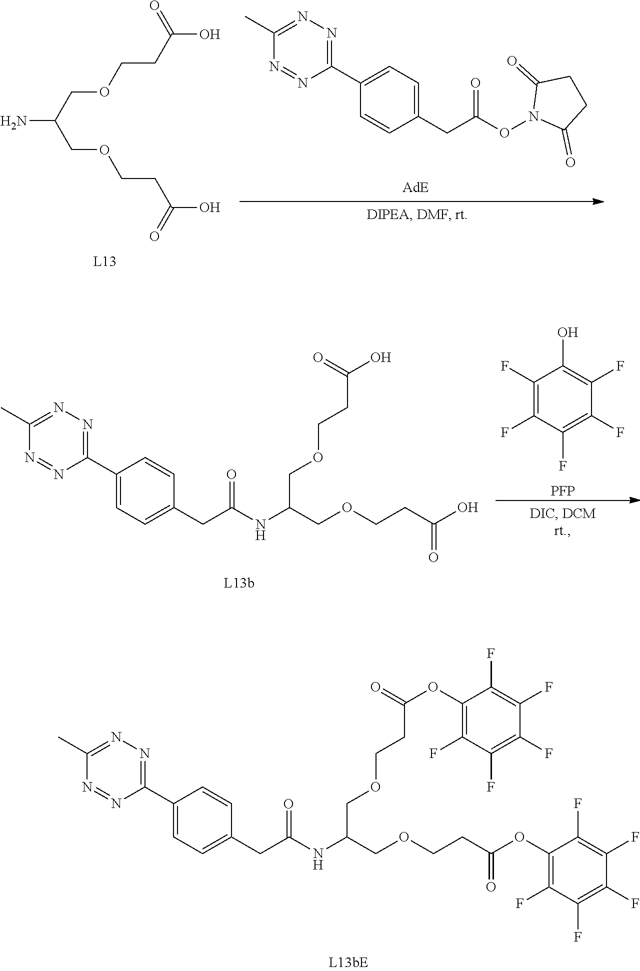

To a stirred mixture of compound AdE (0.28 g, 0.87 mmol) in DMF (10 ml-) were added commercial compound L13 (0.21 g, 0.87 mmol) and DIPEA (0.34 g, 2.6 mmol), and the reaction mixture was stirred at room temperature for an hour. Reaction was monitored by LCMS. The resulting mixture was directly separated by reversed phase flash chromatography (0-100% acetonitrile in aq. TFA (0.1%)) to give compound L13b (65 mg, 17% yield) as a red solid. ESI m/z 448 (M+H)⁺.

To a solution of compound L13b (65 mg, 0.15 mmol) in DCM (10 mL) were added pentafluorophenol (PFP) (54 mg, 0.29 mmol) and N,N′-diisopropylcarbodiimide (DIC) (37 mg, 0.29 mmol), and the mixture was stirred at room temperature for 2 hours. Reaction was monitored by LCMS. The resulting solution was concentrated in vacuo to give crude compound L13bE (65 mg, 57% yield) as a red solid, which was used for the next step without further purification. ESI m/z 780 (M+H)⁺.

### 2,3,4,5,6-pentafluorophenyl 2-{2-[4-(6-methyl-1,2,4,5-tetrazin-3-yl)phenyl]-N-[2-oxo-2-(2,3,4,5,6-pentafluorophenoxy)ethyl]acetamido}acetate (15bE)

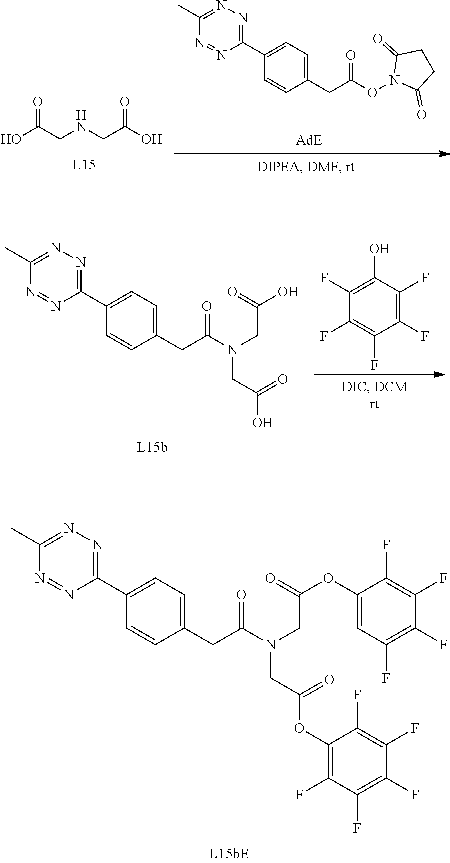 [SMILES: O=C(O)CNCC(=O)O ; Cc1nnc(-c2ccc(CC(=O)ON3C(=O)CCC3=O)cc2)nn1 ; Cc1nnc(-c2ccc(CC(=O)N(CC(=O)O)CC(=O)O)cc2)nn1 ; Oc1c(F)c(F)c(F)c(F)c1F ; Cc1nnc(-c2ccc(CC(=O)N(CC(=O)Oc3cc(F)c(F)c(F)c3F)CC(=O)Oc3c(F)c(F)c(F)c(F)c3F)cc2)nn1]

Following the similar procedure of L13bE except using L15 instead of L13, compound L15bE (30 mg, 6% yield) was obtained as a red solid. ESI m/z 678 (M+H)⁺.

### Example 17. Synthesis of linker-payload LP13 and LP13C (Scheme 5B)

The synthesis of linker-payload LP13 is described in WO2022015656. linker-payload LP13 and LP13C were prepared as described below (FIG. 5).

### tert-butyl (4S)-4-amino-4-{[(1S)-1-{[(1S)-4-(carbamoylamino)-1-{[4-(hydroxymethyl)phenyl]carbamoyl}butyl]carbamoyl}-2-methylpropyl]carbamoyl}butanoate (13-1)

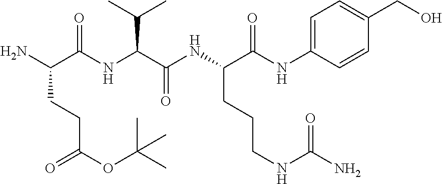 [SMILES: CC(C)[C@H](NC(=O)[C@@H](N)CCC(=O)OC(C)(C)C)C(=O)N[C@@H](CCCNC(N)=O)C(=O)Nc1ccc(CO)cc1]

To a solution of Fmoc-Glu(OtBu)-OH (CAS: 71989-18-9, 1.6 g, 3.8 mmol) in DMF (10 mL) were added HATU (2.9 g, 7.7 mmol) and DIPEA (0.99 g, 7.7 mmol), and the mixture was stirred at room temperature for 15 minutes before the addition of vcPAB (CAS: 159857-79-1, 1.6 g, 4.2 mmol). The reaction mixture was stirred at room temperature for an hour, which was monitored by LCMS. The resulting mixture was separated by reversed phase flash chromatography (0-100% acetonitrile in aq. ammonium bicarbonate (10 mM)) to give Fmoc-13-1 (1.8 g, ESI m/z 787 (M+H)⁺) as a white solid, which was dissolved in DCM (10 mL). To the solution was added diethylamine (0.65 g, 8.9 mmol), and the reaction mixture was stirred at room temperature for 18 hours until Fmoc was totally removed according to LCMS. The volatiles were removed in vacuo and the residue was purified by reversed phase flash chromatography (10-40% acetonitrile in aq. TFA (0.01%)) to give compound 13-1 (1.0 g, 47% yield) as a white solid. ESI m/z 565 (M+H)⁺.

### tert-butyl (4S)-4-{[(1S)-1-{[(1S)-4-(carbamoylamino)-1-{[4-(hydroxymethyl)phenyl]carbamoyl}butyl]carbamoyl}-2-methylpropyl]carbamoyl}-4-(3-{2-[2-({[(9H-fluoren-9-yl)methoxy]carbonyl}amino)ethoxy]ethoxy}propanamido)butanoate (13-2)

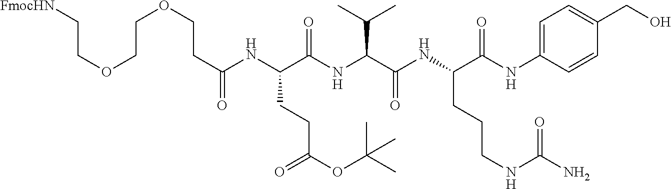

To a solution of Fmoc-PEG₂-acid (0.42 g, 1.0 mmol) in DMF (5 mL) were added HATU (0.48 g, 1.3 mmol) and DIPEA (0.27 g, 2.1 mmol), and the reaction mixture was stirred at room temperature for 15 minutes before the addition of compound 13-1 (0.59 g, 1.0 mmol). The reaction mixture was stirred at room temperature for an hour, which was monitored by LCMS. The resulting mixture as directly separated by reversed phase flash chromatography (0-100% acetonitrile in aq. ammonium bicarbonate (10 mM)) to give compound 13-2 (0.73 g, 73% yield) as a white solid. ESI m/z 946 (M+H)⁺.

### (4S)-4-{[(1S)-1-{[(1S)-4-(carbamoylamino)-1-{[4-({[(4-nitrophenoxy)carbonyl]oxy}methyl)phenyl]carbamoyl}butyl]carbamoyl}-2-methylpropyl]carbamoyl}-4-(3-{2-[2-({[(9H-fluoren-9-yl)methoxy]carbonyl}amino)ethoxy]ethoxy}propanamido)butanoic acid (13-3)

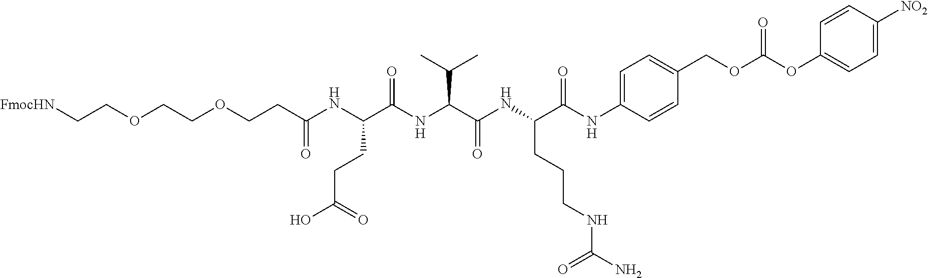

To a solution of compound 13-2 (0.50 g, 0.53 mmol) in DMF (5 mL) were added DMAP (0.13 g, 1.1 mmol), DIPEA (0.68 g, 5.3 mmol), and bis(4-nitrophenyl) carbonate (1.6 g, 5.3 mmol), and the reaction mixture was stirred at room temperature for an hour, which was monitored by LCMS. The resulting mixture was directly separated by reversed phase flash chromatography (40-80% acetonitrile in water) to give a white solid (0.44 g, ESI m/z 1112 (M+H)⁺), which was dissolved in acetonitrile (4 mL). To the solution was added a solution of hydrochloride in ethyl acetate (4 N, 4 mL) at 0° C. The reaction mixture was stirred at 0° C. for 2 hours, which was monitored by LCMS. The volatiles were removed in vacuo at room temperature and the residue was purified by reversed phase flash chromatography (0-100% acetonitrile in aq. ammonium bicarbonate (10 mM)) to give compound 13-3 (0.20 g, 36% yield) as a light yellow solid. ESI m/z 1055 (M+H)⁺.

### (4S)-4-{3-[2-(2-aminoethoxy)ethoxy]propanamido}-4-{[(1S)-1-{[(1S)-4-(carbamoylamino)-1-{[4-({[({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamoyl]oxy}methyl)phenyl]carbamoyl}butyl]carbamoyl}-2-methylpropyl]carbamoyl}butanoic acid (13-4)

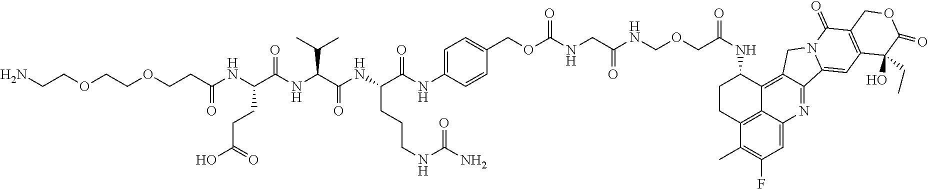 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)[C@H](CCC(=O)O)NC(=O)CCOCCOCCN)C(C)C)cc1)CC3]

To a solution of compound 13-3 (0.11 g, 0.10 mmol) in DMF (2 mL) were added ProDXd (70 mg, 0.12 mmol) and DIPEA (26 mg, 0.20 mmol). The reaction mixture was stirred at room temperature for an hour, which was monitored by LCMS. The resulting mixture was directly separated by reversed phase flash chromatography (30-75% acetonitrile in water) to give Fmoc-13-4 (0.15 g, ESI m/z 749 (M/2+H)⁺) as a yellow solid, which was dissolved in DMF (2 mL). To the solution was added diethylamine (26 mg, 0.35 mmol), and the reaction mixture was stirred at room temperature for 2 hours, which was monitored by LCMS. The volatiles were removed in vacuo and the residual solution was separated by reversed phase flash chromatography (0-100% acetonitrile in aq. ammonium bicarbonate (10 mM)) to give compound 13-4 (85 mg, 67% yield from 13-2, 56% yield from ProDXd) as a white solid. ESI m/z 637 (M/2+H)⁺. (4S)-4-{[(1S)-1-{[(1S)-4-(carbamoylamino)-1-{[4-({[({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamoyl]oxy}methyl)phenyl]carbamoyl}butyl]carbamoyl}-2-methylpropyl]carbamoyl}-4-(3-{2-[2-(3-{3-[2-({2-[2-(2-{[(1S)-1-{[(1S)-1-{[(1S)-4-(carbamoylamino)-1-{[4-({[({[({[(10S,23S)-10-ethyl-18-fluoro-10-hydroxy-19-methyl-5,9-dioxo-8-oxa-4,15-diazahexacyclo[14.7.1.0²,4.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamoyl]oxy}methyl)phenyl]carbamoyl}butyl]carbamoyl}-2-methylpropyl]carbamoyl}-3-carboxypropyl]carbamoyl}ethoxy)ethoxy]ethyl}carbamoyl)ethoxy]-2-[3-(2-{2-[2-(cyclooct-2-yn-1-yloxy)acetamido]ethoxy}ethoxy)propanamido]propoxy}propanamido)ethoxy]ethoxy}propanamido)butanoic acid (LP13)

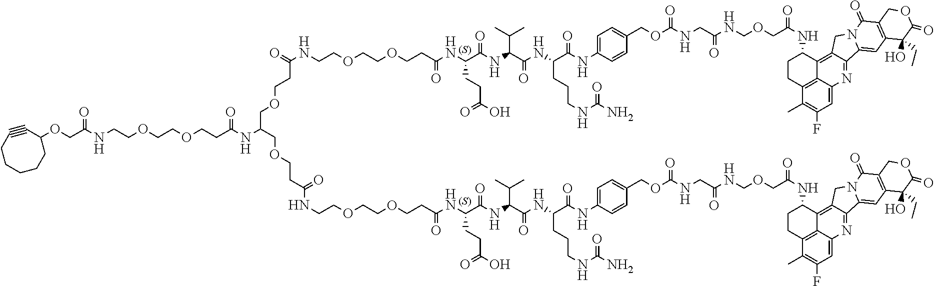

To a solution of compound 13-4 (90 mg, 71 μmol) in DMF (2 mL) were added compound L13aE (24 mg, 27 μmol) and DIPEA (21 mg, 0.16 mmol), and the reaction mixture was stirred at room temperature for 2 hours, which was monitored by LCMS. The resulting mixture was separated by reversed phase flash chromatography (0-100% acetonitrile in aq. ammonium bicarbonate (10 mM)) to give LP13 (45 mg, 21% yield) as a white solid. ESI m/z 1024 (M/3+H)+. ¹H NMR (400 MHz, DMSO_{d6}) δ 10.04 (s, 2H), 8.80 (t, J=6.5 Hz, 2H), 8.51 (d, J=8.8 Hz, 2H), 8.19 (d, J=7.2 Hz, 2H), 8.10 (d, J=7.8 Hz, 2H), 7.93 (t, J=5.4 Hz, 2H), 7.76 (s, 1H), 7.78-7.73 (m, 4H), 7.65-7.55 (m, 5H), 7.42 (d, J=5.6 Hz, 2H), 7.31 (s, 2H), 7.27 (d, J=8.4 Hz, 4H), 6.54 (s, 2H), 6.01 (br s, 2H), 5.62-5.57 (m, 2H), 5.45-5.36 (m, 8H), 5.26-5.10 (m, 4H), 4.92 (s, 4H), 4.63 (d, J=6.5 Hz, 4H), 4.41-4.30 (m, 4H), 4.29-4.25 (m, 1H), 4.22-4.15 (m, 2H), 4.02 (s, 4H), 3.89-3.84 (m, 1H), 3.78-3.73 (m, 1H), 3.66-3.54 (m, 17H), 3.50-3.14 (m, 22H), 3.08-2.99 (m, 2H), 2.96-2.92 (m, 2H), 2.38 (s, 8H), 2.36-2.29 (m, 10H), 2.25-2.13 (m, 12H), 1.99-1.94 (m, 2H), 1.90-1.81 (m, 8H), 1.76-1.67 (m, 6H), 1.61-1.55 (m, 4H), 1.46-1.35 (m, 6H), 0.89-0.81 (m, 22H) ppm.

### LP13C

### (4S)-4-{[(1S)-1-{[(1S)-4-(carbamoylamino)-1-{[4-({[({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamoyl]oxy}methyl)phenyl]carbamoyl}butyl]carbamoyl}-2-methylpropyl]carbamoyl}-4-(3-{2-[2-(3-{3-[2-({2-[2-(2-{[(1S)-1-{[(1S)-1-{[(1S)-4-(carbamoylamino)-1-{[4-({[({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamoyl]oxy}methyl)phenyl]carbamoyl}butyl]carbamoyl}-2-methylpropyl]carbamoyl}-3-carboxypropyl]carbamoyl}ethoxy)ethoxy]ethyl}carbamoyl)ethoxy]-2-{2-[4-(6-methyl-1,2,4,5-tetrazin-3-yl)phenyl]acetamido}propoxy}propanamido)ethoxy]ethoxy}propanamido)butanoic acid (LP13C)

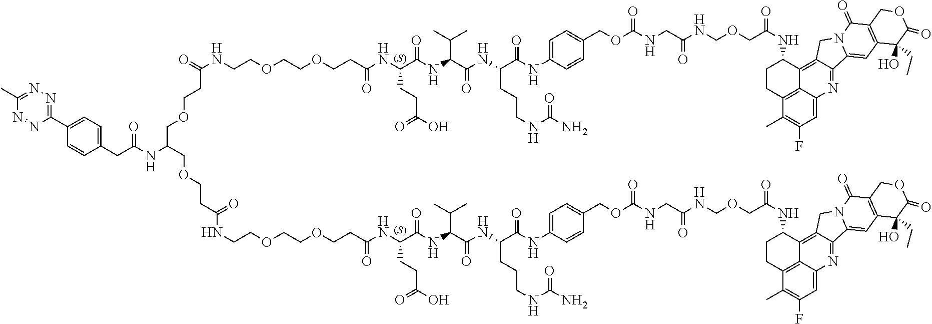

Following the similar procedure as LP13 except starting from L13bE (65 mg, 83 μmol) instead of L13aE, linker-payload LP13C (99 mg, 40% yield) was obtained as a red solid. ESI m/z 1479 (M/2+H)⁺. ¹H NMR (400 MHz, DMSO_{d6}) δ 10.04 (s, 2H), 8.80 (t, J=6.3 Hz, 2H), 8.50 (d, J=8.6 Hz, 2H), 8.39 (d, J=8.2 Hz, 2H), 8.20-8.12 (m, 3H), 8.08 (d, J=7.8 Hz, 2H), 7.94 (t, J=5.5 Hz, 2H), 7.77 (t, J=10.1 Hz, 4H), 7.61-7.49 (m, 6H), 7.43 (t, J=5.9 Hz, 2H), 7.32-7.21 (m, 6H), 6.53 (s, 2H), 5.98 (t, J=5.5 Hz, 2H), 5.63-5.56 (m, 2H), 5.48-5.35 (m, 8H), 5.19 (s, 4H), 4.92 (s, 4H), 4.62 (d, J=6.5 Hz, 4H), 4.41-4.30 (m, 4H), 4.21-4.16 (m, 2H), 4.01 (s, 4H), 3.98-3.92 (m, 1H), 3.66-3.52 (m, 16H), 3.47 (s, 12H), 3.21-3.17 (m, 6H), 2.98 (s, 3H), 2.26-2.14 (m, 9H), 2.08 (s, 9H), 2.03-1.79 (m, 12H), 1.77-1.52 (m, 9H), 1.50-1.34 (m, 6H), 1.24 (s, 6H), 0.88-0.80 (m, 18H) ppm.

### Example 18. Synthesis of LP15 and LP15C

LP15 was prepared as shown in Scheme 5C.

### (2S)-2-[2-(2-Aminoacetamido)acetamido]-N-({[({[(10S,23S)-10-ethyl-18-fluoro-10-hydroxy-19-methyl-5,9-dioxo-8-oxa-4,15-diazahexacyclo[14.7.1.02,14.04,13.06,11.020,24]tetracosa-1,6(11),12,14,16,18,20(24)-heptaen-23-yl]carbamoyl}methoxy)methyl]carbamoyl}methyl)-3-phenylpropanamide (15-1)

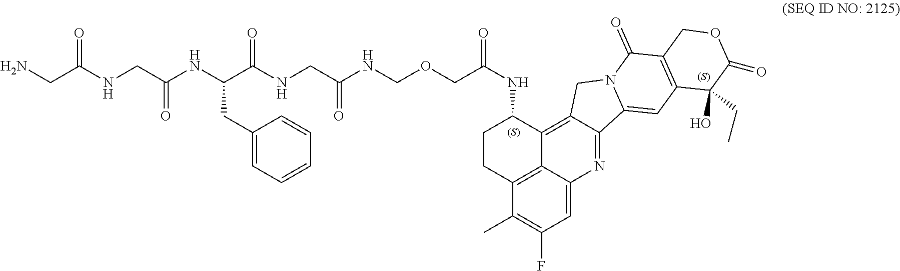

To a yellow solution of Fmoc-Gly-Gly-Phe-OH (CAS: 160036-44-2, 50 mg, 0.10 mmol) in dry DMF (1 mL) was added HATU (46 mg, 0.12 mmol), and the mixture was stirred at room temperature for 5 minutes until the mixture turned clear. To the mixture was then added a solution of ProDXd (TFA salt, 69 mg, 0.10 mmol) and DIPEA (39 mg, 0.30 mmol) in dry DMF (1 mL). The reaction mixture was stirred at room temperature for 2 hours, which was monitored by LCMS. To the resulting solution was added diethylamine (0.2 mL), and the reaction mixture was stirred at room temperature for half an hour, which was monitored by LCMS. The resulting mixture was separated by reversed phase flash chromatography (0-50% acetonitrile in aq. TFA (0.01%)) to give compound 15-1 (30 mg, 36% yield) as a white solid. ESI m/z: 841 (M+H)⁺. ¹H NMR (400 MHz, DMSO_{d6}) δ 8.70 (t, J=5.2 Hz, 1H), 8.54 (dd, J=6.8, 2.4 Hz, 1H), 8.49 (t, J=4.4 Hz, 1H), 8.39 (t, J=4.4 Hz, 1H), 8.35-8.31 (m, 1H), 8.28 (br s, 2H), 7.79 (d, J=8.8 Hz, 1H), 7.32 (s, 1H), 7.27-7.20 (m, 4H), 7.19-7.16 (m, 1H), 6.54 (s, 1H), 5.62-5.58 (m, 1H), 5.42 (s, 2H), 5.21 (s, 2H), 4.65 (d, J=5.2 Hz, 2H), 4.57-4.50 (m, 1H), 4.03 (s, 2H), 3.89-3.84 (m, 1H), 3.79-3.68 (m, 3H), 3.58 (s, 2H), 3.25-3.11 (m, 2H), 3.06-3.01 (m, 1H), 2.78-2.72 (m, 1H), 2.39 (s, 3H), 2.23-2.13 (m, 2H), 1.92-1.81 (m, 2H), 1.28-1.24 (m, 1H), 0.87 (t, J=5.6 Hz, 3H) ppm.

### (2S)-2-[2-(2-{3-[2-(2-Aminoethoxy)ethoxy]propanamido}acetamido)acetamido]-N-({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamoyl}methyl)-3-phenylpropanamide (15-2)

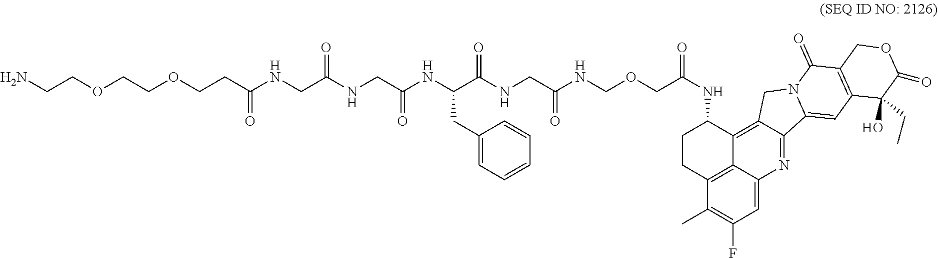 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCOCCOCCN)CC3]

To a solution of compound 15-1 (58 mg, 69 μmol) in DMF (5 mL) were successively added compound Fmoc-PEG₂-acid (CAS: 872679-70-4, 28 mg, 69 μmol), DIPEA (18 mg, 0.14 mmol) and HATU (40 mg, 0.10 mmol), and the reaction mixture was stirred at room temperature for 4 hours, which was monitored by LCMS. The resulting mixture was separated by prep-HPLC (0-100% acetonitrile in aq. TFA (0.05%)) to give a white solid (54 mg, ESI m/z: 729.3 (M−M_{Dxd}+H)+), which was dissolved in DMF (5 mL). To the solution was added diethylamine (16 mg, 0.22 mmol), and the mixture was stirred at room temperature for 2 hours until Fmoc was totally removed according to LCMS. The resulting mixture was directly purified by reversed phase flash chromatography (0-100% acetonitrile in aq. TFA (0.01%)) to give compound 15-2 (40 mg, 57% yield) as a white solid. ESI m/z: 1001 (M+H)+, 501 (M/2+H)⁺.

### 1-[2-(Cyclooct-2-yn-1-yloxy)acetamido]-N-(2-{2-[2-({[({[(1S)-1-[({[({[(10S,23S)-10-ethyl-18-fluoro-10-hydroxy-19-methyl-5,9-dioxo-8-oxa-4,15-diazahexacyclo[14.7.1.02,14.04,13.06,11.020,24]tetracosa-1,6(11),12,14,16,18,20(24)-heptaen-23-yl]carbamoyl}methoxy)methyl]carbamoyl}methyl)carbamoyl]-2-phenylethyl]carbamoyl}methyl)carbamoyl]methyl}carbamoyl)ethoxy]ethoxy}ethyl)-12-{[(2-{2-[2-({[({[(1S)-1-[({[({[(10S,23S)-10-ethyl-18-fluoro-10-hydroxy-19-methyl-5,9-dioxo-8-oxa-4,15-diazahexacyclo[14.7.1.02,14.04,13.06,11.020,24]tetracosa-1,6(11),12,14,16,18,20(24)-heptaen-23-yl]carbamoyl}methoxy)methyl]carbamoyl}methyl)carbamoyl]-2-phenylethyl]carbamoyl}methyl)carbamoyl]methyl}carbamoyl)ethoxy]ethoxy}ethyl)carbamoyl]methyl}-3,6,9-trioxa-12-azatetradecan-14-amide (LP15)

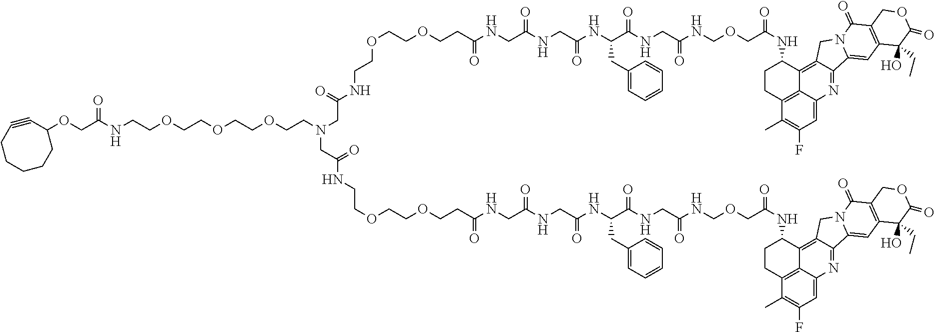 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCOCCOCCNC(=O)CN(CCOCCOCCOCCNC(=O)COC1C#CCCCCC1)CC(=O)NCCOCCOCCC(=O)NCC(=O)NCC(=O)N[C@@H](Cc1ccccc1)C(=O)NCC(=O)NCOCC(=O)N[C@H]1CCc2c(C)c(F)cc4nc5c(c1c24)Cn1c-5cc2c(c1=O)COC(=O)[C@]2(O)CC)CC3]

To a mixture of compound 15-2 (40 mg, 40 μmol) in DMF (5 mL) were added compound L15aE (16 mg, 20 μmol) and DIPEA (8.0 mg, 62 μmol), and the reaction mixture was stirred at room temperature for an hour, which was monitored by LCMS. the resulting mixture was directly separated by reversed phase flash chromatography (0-100% acetonitrile in aq. TFA (0.01%)) to give linker-payload LP15 (12 mg, 24% yield) as a white solid. ESI m/z: 813 (M/3+H)+. ¹H NMR (400 MHz, DMSO_{d6}) δ 8.64 (t, J=6.6 Hz, 2H), 8.52 (d, J=8.7 Hz, 2H), 8.31 (t, J=5.9 Hz, 2H), 8.19-8.09 (m, 4H), 8.05-7.98 (m, 3H), 7.77 (d, J=10.9 Hz, 2H), 7.65-7.56 (m, 2H), 7.31 (s, 2H), 7.29-7.12 (m, 8H), 6.53 (s, 2H), 5.65-5.56 (m, 3H), 5.42 (s, 3H), 5.20-5.15 (m, 3H), 4.70-4.60 (m, 3H), 4.50-4.44 (m, 2H), 4.30-4.22 (m, 2H), 4.02 (s, 3H), 3.86 (d, J=14.8 Hz, 2H), 3.78-3.66 (m, 9H), 3.63-3.55 (m, 6H), 3.53-3.40 (m, 27H), 3.27-3.19 (m, 6H), 3.15 (s, 4H), 3.09-3.00 (m, 2H), 2.81-2.72 (m, 2H), 2.70-2.63 (m, 2H), 2.42-2.32 (m, 8H), 2.25-2.11 (m, 6H), 2.09-1.97 (m, 3H), 1.94-1.68 (m, 9H), 1.63-1.51 (m, 3H), 1.43-1.34 (m, 2H), 1.29-1.20 (m, 3H), 0.87 (t, J=7.3 Hz, 6H) ppm. ¹⁹F NMR (376 MHz, DMSO_{d6}) 5-111.24 ppm. (no TFA signal).

### LP15C

### (2S)-N-({[({[(10S,23S)-10-ethyl-18-fluoro-10-hydroxy-19-methyl-5,9-dioxo-8-oxa-4,15-diazahexacyclo[14.7.1.0²,4.0^{4,13}.0^{6,11}.0^{20,24}]tetracosa-1,6(11),12,14,16,18,20(24)-heptaen-23-yl]carbamoyl}methoxy)methyl]carbamoyl}methyl)-2-(2-{2-[3-(2-{2-[2-(N-{[(2-{2-[2-({[({[(1S)-1-[({[({[(10S,23S)-10-ethyl-18-fluoro-10-hydroxy-19-methyl-5,9-dioxo-8-oxa-4,15-diazahexacyclo[14.7.1.0²,4.0^{4,13}.0^{6,11}.0^{20,24}]tetracosa-1,6(11),12,14,16,18,20(24)-heptaen-23-yl]carbamoyl}methoxy)methyl]carbamoyl}methyl)carbamoyl]-2-phenylethyl]carbamoyl}methyl)carbamoyl]methyl}carbamoyl)ethoxy]ethoxy}ethyl)carbamoyl]methyl}-2-[4-(6-methyl-1,2,4,5-tetrazin-3-yl)phenyl]acetamido)acetamido]ethoxy}ethoxy)propanamido]acetamido}acetamido)-3-phenylpropanamide (LP15C)

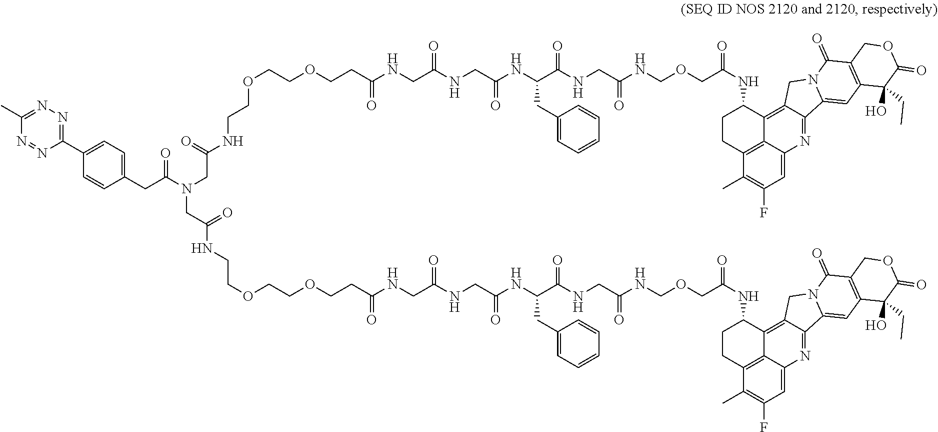 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCOCCOCCNC(=O)CN(CC(=O)NCCOCCOCCC(=O)NCC(=O)NCC(=O)N[C@@H](Cc1ccccc1)C(=O)NCC(=O)NCOCC(=O)N[C@H]1CCc2c(C)c(F)cc4nc5c(c1c24)Cn1c-5cc2c(c1=O)COC(=O)[C@]2(O)CC)C(=O)Cc1ccc(-c2nnc(C)nn2)cc1)CC3]

Following the similar procedures as LP15 except starting from L15bE (16 mg, 21 μmol) instead of L15aE, linker-payload LP15C (8 mg, 17% yield) was obtained as a red solid. ESI m/z 770 (M/3+H)+.

### Example 19. Synthesis of linker-DXd

LP16 was prepared as shown in Scheme 5D.

### (2S)-2-[(2S)-2-amino-3-methylbutanamido]-5-(carbamoylamino)-N-[4-({[({[(10S,23S)-10-ethyl-18-fluoro-10-hydroxy-19-methyl-5,9-dioxo-8-oxa-4,15-diazahexacyclo[14.7.1.0²,4.0^{4,13}.0^{6,11}.0^{20,24}]tetracosa-1,6(11),12,14,16,18,20(24)-heptaen-23-yl]carbamoyl}methoxy)carbonyl]oxy}methyl)phenyl]pentanamide (16-2)

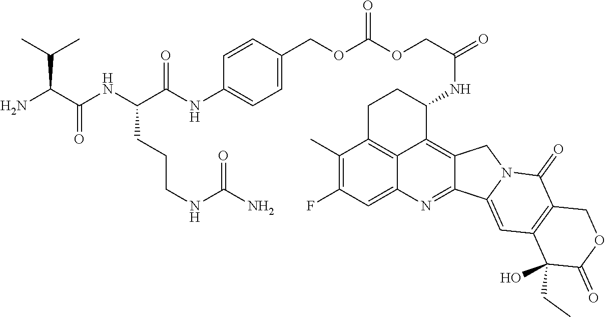 [SMILES: CC[C@@]1(O)C(=O)OCc2c1cc1n(c2=O)Cc2c-1nc1cc(F)c(C)c3c1c2[C@@H](NC(=O)COC(=O)OCc1ccc(NC(=O)[C@H](CCCNC(N)=O)NC(=O)[C@@H](N)C(C)C)cc1)CC3]

To a solution of DXd (100 mg, 0.20 mmol) in THF (5 mL) were added bis(4-nitrophenyl) carbonate (0.14 g, 0.46 mmol) and DIPEA (0.12 g, 0.92 mmol) successively at room temperature, and the reaction mixture was then stirred at this temperature for 16 hours. Most of DXd was consumed according to LCMS. The volatiles were then removed and the residue was purified by silica gel column chromatography (5-90% ethyl acetate in petroleum ether) to give activated ester 16-1 (80 g, ESI m/z: 659 (M+H)⁺) as a light yellow solid, which was dissolved in dry DMF (4 mL). To the DMF solution were added Boc-vc-PAB-OH (100 mg, 0.21 mmol), DMAP (40 mg, 0.32 mmol), and pyridine (40 mg, 0.51 mmol), successively. The mixture was stirred at room temperature for 16 hours. The reaction mixture was directly purified by reversed phase flash chromatography (5-95% acetonitrile in water) to give Boc-16-2 (70 mg, ESI m/z: 1020 (M+Na)+) as a white solid, which was dissolved in DCM (10 mL). To the DCM solution was added TFA (1 mL) dropwise at 0° C. After stirred at room temperature for 1.5 hours until the Boc was totally removed, which was monitored by LCMS, the resulting mixture was concentrated in vacuo to give crude title product 16-2 (26 mg, 13% yield from DXd, TFA salt) as light yellow solid. ESI m/z: 899 (M+H)⁺.

### N-[(1S)-1-{[(1S)-4-(carbamoylamino)-1-{[4-({[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)carbonyl]oxy}methyl)phenyl]carbamoyl}butyl]carbamoyl}-2-methylpropyl]-1-[2-(cyclooct-2-yn-1-yloxy)acetamido]-3,6,9,12-tetraoxapentadecan-15-amide (LP16)

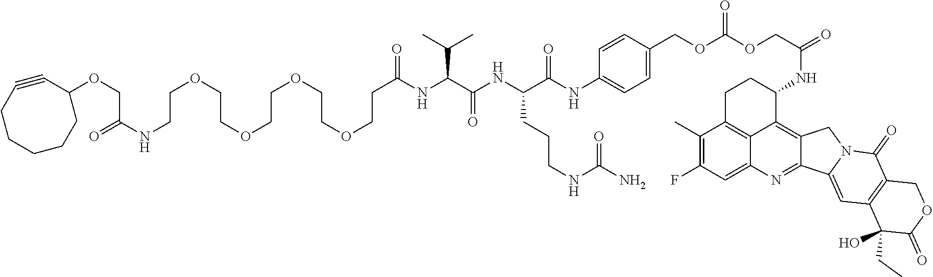 [SMILES: CC[C@@]1(O)C(=O)OCc2c1cc1n(c2=O)Cc2c-1nc1cc(F)c(C)c3c1c2[C@@H](NC(=O)COC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

To a solution of compound 16-2 (25 mg, 25 μmol) in dry DMF (1.5 mL) were added compound Ba (14 mg, 27 μmol) and DIPEA (14 mg, 0.11 mmol) successively, and the reaction mixture was stirred at room temperature for an hour, which was monitored by LCMS. The resulting mixture was directly separated by prep-HPLC (5-95% acetonitrile in aq. formic acid (0.05%)) to give linker-payload LP16 (3 mg, 9% yield) as a white solid. ESI m/z 656 (M/2+H)⁺.

LP17 was prepared as shown in Scheme 5E.

### 2-({[(4-azidophenyl)methoxy]carbonyl}amino)acetic acid (17-1)

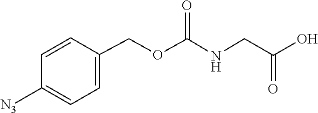 [SMILES: [N-]=[N+]=Nc1ccc(COC(=O)NCC(=O)O)cc1]

To a solution of 4-aminobenzyl alcohol (10 g, 81 mmol) and azido(trimethyl)silane (11 g, 97 mmol, 13 mL) in acetonitrile (600 mL) was added tert-butyl nitrite (13 g, 0.12 mol, 15 mL) dropwise at 0° C. The mixture was stirred at room temperature for 2 hours before the addition of a solution of bis(4-nitrophenyl) carbonate (32 g, 0.11 mol) and DIPEA (21 g, 0.16 mol, 28 mL) in THF (300 mL), and the mixture was stirred at room temperature for 12 hours which was monitored by TLC (25% ethyl acetate in petroleum ether). The volatiles were removed in vacuo and the residue was dissolved in acetonitrile (500 mL). To the solution were added glycine (15 g, 0.20 mol) and aq. sodium bicarbonate (0.8 M, 250 mL, 0.2 mol) dropwise, and the mixture was stirred at room temperature for 16 hours, which was monitored by TLC (25% ethyl acetate in petroleum ether). The resulting mixture was washed with ethyl acetate (150 mL×6). And the aqueous solution was acidified with conc. HCl to pH 2-3 and was then extracted with ethyl acetate (150 mL×3). The combined organic solution was dried over anhydrous sodium sulfate and concentrated in vacuo to give 17-1 (13 g, 61% yield) as a brown solid, which was used in the next step without further purification. ¹H NMR (400 MHz, MeODd₄) δ 7.46-7.34 (m, 2H), 7.12-6.97 (m, 2H), 5.08 (s, 2H), 3.83 (s, 2H) ppm.

### ({[(4-azidophenyl)methoxy]carbonyl}amino)methyl acetate (17-2)

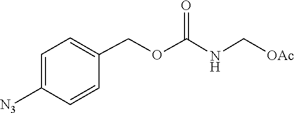 [SMILES: CC(=O)OCNC(=O)OCc1ccc(N=[N+]=[N-])cc1]

To a solution of compound 17-1 (13 g, 51 mmol) in THF (150 mL) lead acetate (45 g, 0.10 mol) and copper diacetate (0.93 g, 5.1 mmol) were added. The reaction mixture was stirred at 40° C. for an hour. The reaction was monitored by TLC (25% ethyl acetate in petroleum ether). The reaction was quenched with water (200 mL) and extracted with ethyl acetate (200 mL×2). The combined organic solution was washed with brine (150 mL×2), dried over anhydrous sodium sulfate, and concentrated in vacuo. The residue was purified by silica gel flash chromatography (0-25% ethyl acetate in petroleum ether) to give compound 17-2 (7.8 g, 55% yield) as yellow oil. ¹H NMR (400 MHz, CDCl₃) δ 7.36 (br d, J=8.2 Hz, 2H), 7.03 (d, J=8.4 Hz, 2H), 5.93 (br s, 1H), 5.21 (br d, J=7.5 Hz, 2H), 5.11 (s, 2H), 2.07 (s, 3H) ppm.

### 2-[({[(4-azidophenyl)methoxy]carbonyl}amino)methoxy]acetic acid (17-3)

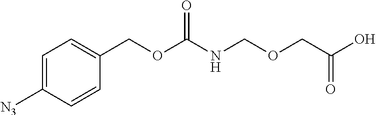 [SMILES: [N-]=[N+]=Nc1ccc(COC(=O)NCOCC(=O)O)cc1]

To a solution of 17-2 (0.20 g, 0.76 mmol) in DCM (3 mL) were added PPTS (38 mg, 0.15 mmol) and hydroxyacetic acid (0.17 g, 2.3 mmol), and the reaction mixture was stirred in a sealed tube at 50° C. for 16 hours, which was monitored by LCMS. The resulting mixture was cooled and the volatiles were removed in vacuo. The residue was purified by prep-HPLC (0-100% acetonitrile in aq. TFA (0.01%)) to give compound 17-3 (0.10 g, 47% yield) as a yellow solid. ESI m/z: 303 (M+Na)⁺.

### {4-[(2S)-2-[(2S)-2-amino-3-methylbutanamido]propanamido]phenyl}methyl N-[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamate (17-4)

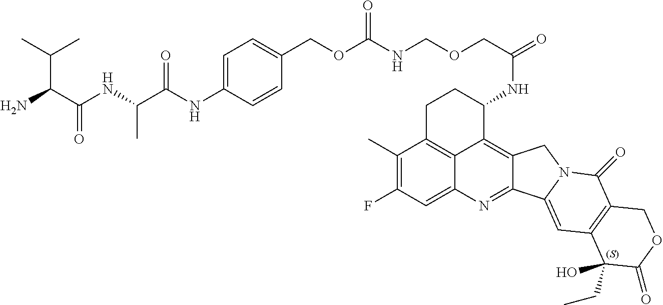

To a yellow solution of compound 17-3 (50 mg, 0.13 mmol) and DIPEA (49 mg, 0.38 mmol) in dry DMF (1.5 mL) was added HATU (59 mg, 0.15 mmol), and the mixture was stirred at room temperature for half an hour before the addition of exatecan (60 mg, 0.11 mmol). The reaction mixture was stirred at room temperature for 2 hours, which was monitored by LCMS. The resulting mixture was directly separated by prep-HPLC (5-95% acetonitrile in aq. TFA (0.01%)) to give compound 17-3b (58 mg, 65% yield) as a pale-yellow solid. ESI m/z: 698 (M+H)+.

To a 25 mL vial, charged with a stir bar and THF (3 mL), were added 17-3b (42 mg, 52 μmol), 4A molecular sieves (0.50 g), and then trimethylphosphine (0.16 mL, 0.16 mmol). The reaction mixture was stirred for 5 minutes before the addition of Fmoc-Val-Ala-OPFP (33 mg, 57 μmol). The reaction mixture was stirred at room temperature under nitrogen protection for half an hour, which was monitored by LCMS. The resulting mixture was directly separated by prep-HPLC (5-95% acetonitrile in aq. TFA (0.01%)) to give Fmoc-17-4 (53 mg, TFA salt) as a light-yellow solid, which was dissolved in dry DMF (1 mL). To the solution was added diethylamine (0.1 mL). The reaction mixture was stirred at room temperature for an hour until Fmoc was totally removed according to LCMS. The resulting solution was directly separated by prep-HPLC (5-95% acetonitrile in aq. TFA (0.01%)) to give compound 17-4 (28 mg, 57% yield, TFA salt) as a light-yellow solid. ESI m/z: 422 (M/2+H)⁺.

NMR for Fmoc-17-4: ¹H NMR (400 MHz, DMSO_{d6}) δ 10.03 (s, 1H), 8.48-8.29 (m, 1H), 8.26-8.11 (m, 1H), 7.89 (d, J=7.5 Hz, 2H), 7.79-7.72 (m, 3H), 7.70-7.52 (m, 2H), 7.44-7.41 (m, 3H), 7.34-7.25 (m, 3H), 7.20-7.17 (m, 2H), 6.54 (d, J=9.0 Hz, 1H), 5.59 (s, 2H), 5.42 (s, 2H), 5.21 (s, 1H), 4.87 (s, 1H), 4.54 (d, J=6.5 Hz, 2H), 4.41-4.32 (m, 1H), 4.35-4.17 (m, 3H), 4.00 (s, 1H), 3.98-3.83 (m, 1H), 3.15 (s, 2H), 2.38 (s, 3H), 2.18-2.11 (m, 3H), 2.09-1.95 (m, 2H), 1.93-1.71 (m, 2H), 1.30-1.14 (m, 6H), 0.89-0.77 (m, 9H) ppm. ¹⁹F NMR (376 MHz, DMSO_{d6}) δ −73.45, −111.33 ppm.

NMR for 17-4: ¹H NMR (400 MHz, DMSO_{d6}) δ 10.20 (br, 1H), 8.84-8.67 (m, 1H), 8.46 (br s, 1H), 8.21 (br s, 2H), 8.06 (s, 2H), 7.80 (d, J=10.5 Hz, 1H), 7.65-7.50 (m, 2H), 7.31 (s, 1H), 7.23 (t, J=8.2 Hz, 1H), 6.53 (s, 1H), 5.60 (s, 1H), 5.42 (s, 2H), 5.22 (s, 1H), 4.88 (d, J=5.8 Hz, 2H), 4.60-4.45 (m, 3H), 4.00 (s, 1H), 3.61 (br s, 1H), 3.17 (br s, 1H), 2.93-2.90 (m, 2H), 2.39 (s, 3H), 2.33-2.06 (m, 2H), 1.93-1.76 (m, 2H), 1.34 (t, J=7.4 Hz, 3H), 1.16 (t, J=7.3 Hz, 3H), 0.97-0.93 (m, 6H), 0.85 (t, J=7.4 Hz, 3H) ppm. ¹⁹F NMR (376 MHz, DMSO_{d6}) δ −73.44, −111.33 ppm.

### {4-[(2S)-2-[(2S)-2-{1-[2-(cyclooct-2-yn-1-yloxy)acetamido]-3,6,9,12-tetraoxapentadecan-15-amido}-3-methylbutanamido]propanamido]phenyl}methyl N-[({[(10S,23S)-10-ethyl-18-fluoro-10-hydroxy-19-methyl-5,9-dioxo-8-oxa-4,15-diazahexacyclo[14.7.1.0^{2,14}.0^{4,13}.0^{6,11}.0^{20,24}]tetracosa-1,6(11),12,14,16,18,20(24)-heptaen-23-yl]carbamoyl}methoxy)methyl]carbamate (LP17)

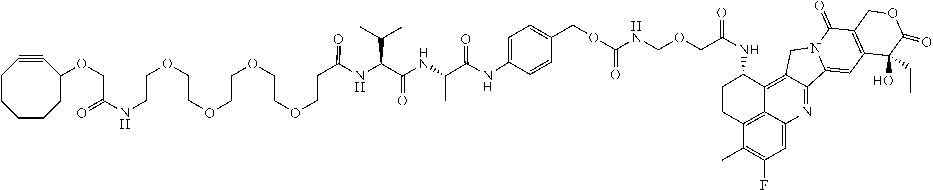 [SMILES: CC[C@@]1(O)C(=O)OCc2c1cc1n(c2=O)Cc2c-1nc1cc(F)c(C)c3c1c2[C@@H](NC(=O)COCNC(=O)OCc1ccc(NC(=O)[C@H](C)NC(=O)[C@@H](NC(=O)CCOCCOCCOCCOCCNC(=O)COC2C#CCCCCC2)C(C)C)cc1)CC3]

To a yellow solution of compound Ba (14 mg, 27 μmol) in dry DMF (1.5 mL) were added DIPEA (14 mg, 0.11 mmol) and compound 17-4 (23 mg, 24 μmol), and the reaction mixture was stirred at room temperature for an hour, which was monitored by LCMS. The resulting mixture was directly separated by prep-HPLC (5-95% acetonitrile in aq. formic acid (0.1%)) to give linker-payload LP17 (9.0 mg, 26% yield) as a white solid. ESI m/z: 1253 (M+H)⁺. ¹H NMR (400 MHz, DMSO_{d6}) δ 9.96 (s, 1H), 8.45 (m, 1H), 8.21 (m, 1H), 7.89 (d, J=8.6 Hz, 1H), 7.79 (d, J=10.9 Hz, 1H), 7.66-7.52 (m, 3H), 7.30 (s, 1H), 7.25-7.19 (m, 2H), 6.53 (s, 1H), 5.59 (s, 1H), 5.42 (s, 2H), 5.22 (s, 2H), 4.86 (s, 2H), 4.54 (d, J=6.7 Hz, 2H), 4.45-4.30 (m, 1H), 4.29-4.08 (m, 2H), 4.00 (s, 2H), 3.87 (d, J=14.8 Hz, 1H), 3.75 (d, J=14.7 Hz, 1H), 3.58 (d, J=6.2 Hz, 2H), 3.51-3.45 (m, 12H), 3.44-3.40 (m, 2H), 3.29-3.21 (m, 2H), 3.16 (br s, 2H), 2.45-2.36 (m, 5H), 2.24-2.02 (m, 5H), 2.02-1.70 (m, 7H), 1.61-1.52 (m, 2H), 1.44-1.22 (m, 5H), 0.92-0.77 (m, 9H) ppm. ¹⁹F NMR (376 MHz, DMSO_{d6}) δ −111.33 ppm.

### Synthesis of Intermediates

### Example 20. Synthesis of intermediate A (Scheme 6) 8,8-dibromobicyclo[5.1.0]octane (A-2)

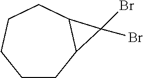

To a stirred hexane (12 mL) was added potassium tert-butoxide (2.3 kg, 21 mol) in batches under nitrogen atmosphere. The mixture was stirred and cooled to −10° C. To the cooled and stirred mixture was added cycloheptene (A-1) (0.80 kg, 8.3 mol, 0.96 L) dropwise during an hour. The resulting suspension was stirred at −10° C. to −5° C. and to the suspension was added a solution of bromoform (3.2 kg, 13 mol, 1.1 L) in hexane (4.0 L) over 1.5 hours, maintaining the temperature between −10° C. to −5° C. The resulting suspension was then allowed to warm to 25° C. and stirred at this temperature for 16 hours until most of A-1 (>90%) was consumed, which was monitored by TLC (eluting with petroleum ether, PMA, R_{f}=0.8). The reaction mixture was quenched by cold water at 20-25° C. and cold aq. hydrochloride (1.0 M, 8.0 L) below 25° C. The resulting biphasic mixture was diluted with hexane (2.4 L) and separated. The aqueous layer was extracted with hexane (2.4 L×2) and the combined organic solution was dried over anhydrous sodium sulfate and concentrated in vacuo to give compound A-2 (1.8 kg, crude, containing 2.4% of A-1) as black oil, which was used for the next step without further purification. ¹H NMR (400 MHz, CDCl₃) δ 2.37-2.20 (m, 2H), 1.95-1.79 (m, 3H), 1.77-1.66 (m, 2H), 1.41-1.31 (m, 2H), 1.25-1.13 (m, 3H) ppm. Alternative ¹H NMR (400 MHz, CDCl₃): 2.19-2.33 (m, 2H, H-1, H-7), 1.77-1.94 (m, 3H), 1.63-1.76 (m, 2H), 1.31-1.40 (m, 2H), 1.11-1.24 (m, 3H) ppm.

| | Raw material | | | | | | | | | |
|---|---|---|---|---|---|---|---|---|---|---|
| | (Equivalent/Volume) | | | Solvent | Temp. | Time | Product A-2 | | | Analytical data |
| # | A-1 | tBuOK | bromoform | hexane | (° C.) | h | Amount | Yield | purity | TLC |
| 1 | 50.0 g | 145 g | 68 mL | 1000 mL | −10~25 | 16 | 0.11 kg | 79% | crude | R_{f} = 0.8 |
| | (1.00 eq.) | (2.5 eq.) | (1.5 eq) | (20 Vol.) | | | | | | (Eluting with petroleum |
| | | | | | | | | | | ether, PMA) |
| 2 | 100.0 g*2 | 290 g | 136 mL | 2000 mL | −10~25 | 16 | 0.45 kg | 81% | crude | R_{f} = 0.8 |
| | (1.00 eq.) | (2.5 eq.) | (1.5 eq) | (20 Vol.) | | | | | | (Eluting with petroleum |
| | | | | | | | | | | ether, PMA) |
| 3 | 125 g*2 | 362 g | 170 mL | 2500 mL | −10~25 | 16 | 0.56 kg | 80% | crude | R_{f} = 0.8 |
| | (1.00 eq.) | (2.5 eq) | (1.5 eq) | (20 Vol.) | | | | | | (Eluting with petroleum |
| | | | | | | | | | | ether, PMA) |
| 4 | 800 g | 2.32 kg | 1.09 L | 2500 mL | −10~25 | 16 | 1.8 kg | 81% | crude | R_{f} = 0.8 |
| | (1.00 eq.) | (2.5 eq) | (1.5 eq) | (20 Vol.) | | | | | | (Eluting with petroleum |
| | | | | | | | | | | ether, PMA) |

### methyl 2-{[(2Z)-2-bromocyclooct-2-en-1-yl]oxy}acetate (A-3)

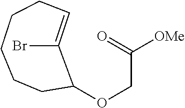 [SMILES: COC(=O)COC1CCCCC/C=C/1Br]

To a solution of compound A-2 (1.8 kg, 6.7 mol) in DCM (3.6 L) was added methyl glycolate (5.0 L, 65 mol) at 25° C. and the reactor was protected from light using aluminium foil. To the reaction mixture was added silver trifluoromethanesulfonate (3.5 kg, 13 mol) in one portion, and the mixture was stirred at 25° C. for an hour, which was monitored by TLC (eluting with petroleum ether, PMA, R_{f}=0.8 for A-2 consumed, R_{f}=0.15 for A-3 formed). The resulting mixture was quenched with sat. aq. sodium bicarbonate (5.0 L) and brine (2.5 L). The silver salts precipitated and was filtered off. The filter cake was washed with MTBE (3.0 L) and the filtrate was diluted with MTBE (3.0 L). The organic layer was washed with water (2.0 L×2), dried over anhydrous sodium sulfate and concentrated in vacuo to give crude A-3 (1.8 kg, crude, 97% yield) as black oil, which was used for the next step without further purification. ¹H NMR (400 MHz, CDCl₃) δ 6.22 (dd, J=11.63, 4.13 Hz, 1H), 4.24 (d, J=16.51 Hz, 1H), 4.16-4.08 (m, 1H), 3.98 (d, J=16.51 Hz, 1H), 3.79-3.72 (m, 3H), 2.84-2.65 (m, 1H), 2.35-2.24 (m, 1H), 2.14-1.86 (m, 4H), 1.77-1.76 (m, 1H), 1.55-1.42 (m, 1H), 1.35-1.24 (m, 1H), 0.88-0.71 (m, 1H) ppm. Alternative ¹H NMR (400 MHz, CDCl₃) δ 6.19 (dd, J=11.6 Hz, 4.1 Hz, 1H, H-2), 4.19 (d, J=16.4 Hz, 1H, H-9a), 4.06-4.12 (m, 1H, H-8), 3.94 (d, J=16.5 Hz, 1H, H-9b), 3.74 (s, 3H, H-11), 2.69 (qd, J=11.8, 5.4 Hz, 1H, H-3a), 2.20-2.32 (m, 1H, H-3b), 1.18-2.10 (m, 7H, H-4, H-5, H-6a, H-7), 0.70-0.88 (m, 1H, H-6b) ppm.

| | Raw material | | | | | | | | |
|---|---|---|---|---|---|---|---|---|---|
| | (Equivalent/Volume) | | | | | | | | |
| | | | Methyl | Solvent | Temp. | Time | Product A-3 | | Analytical data |
| # | A-2 | AgOTf | glycolate | DCM | ° C. | h | Amount | purity | TLC |
| 1 | 0.10 kg | 0.19 kg | 0.29 L | 0.20 L | 25 | 1 | 98 g | crude | R_{f} = 0.43 |
| | | | | | | | | | (petroleum ether/ethyl acetate, |
| | | | | | | | | | v/v = 5, PMA) |
| 2 | 0.45 kg | 0.86 kg | 1.3 L | 0.90 L | 25 | 1 | 0.45 kg | crude | R_{f} = 0.43 |
| | | | | | | | | | (petroleum ether/ethyl acetate, |
| | | | | | | | | | v/v = 5, PMA) |
| 3 | 0.56 kg | 1.1 kg | 1.6 L | 1.2 L | 25 | 1 | 0.55 kg | crude | R_{f} = 0.43 |
| | | | | | | | | | (petroleum ether/ethyl acetate, |
| | | | | | | | | | v/v = 5, PMA) |
| 4 | 1.8 kg | 3.4 kg | 5.1 L | 3.6 L | 25 | 1 | 1.8 kg | crude | R_{f} = 0.43 |
| | | | | | | | | | (petroleum ether/ethyl acetate, |
| | | | | | | | | | v/v = 5, PMA) |

### 2-(cyclooct-2-yn-1-yloxy)acetic acid (A-4, COT)

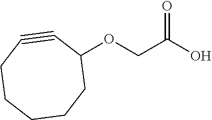 [SMILES: O=C(O)COC1C#CCCCCC1]

To a solution of compound A-3 (1.6 kg, crude) obtained above in DMSO (1.6 L) was added a solution of sodium methoxide in methanol (30%, 0.48 L) dropwise in 15 minutes, maintaining the temperature between 20° C. to 25° C. The resulting solution was stirred at 25° C. for an hour, which was monitored by TLC (eluting with petroleum ether/ethyl acetate, v/v=1, PMA, R_{f}=0.87 for compound A-3 was consumed and R_{f}=0.44 for compound A-4 was formed). The resulting mixture was quenched with cold water (16 L) and diluted with DCM (16 L). The aqueous layer was washed with DCM (4 L). The aqueous solution was acidified with aq. hydrochloride (1 M) to pH<2, and was then extracted with MTBE (8 L×2). The combined MTBE solution was washed with water (8 L), dried over anhydrous sodium sulfate, and concentrated in vacuo. The residue was purified by silica gel column chromatography (eluting with petroleum ether/ethyl acetate, v/v=10/1 to 5/1) to give crude product A-4, which was suspended in hexane (5 L) and stirred at 25° C. for 3 hours. The suspension was centrifuged for 10 minutes, and filtered to collect filter cake. The solid was dried in vacuum to give pure A-4 (0.63 kg, 47% yield in 3 steps from A-1) as a white solid. ESI m/z: 181.1 (M−H)+; ¹H NMR (400 MHz, CDCl₃) δ 10.26 (br s, 1H), 4.44-4.33 (m, 1H), 4.29-4.21 (m, 1H), 4.16 (br s, 1H), 2.31-2.22 (m, 1H), 2.22-2.12 (m, 2H), 2.10-2.00 (m, 1H), 1.97-1.78 (m, 3H), 1.70-1.61 (m, 2H), 1.54-1.43 (m, 1H) ppm. Alternative ¹H NMR (400 MHz, CDCl₃) δ 9.25 (br s, 1H, H-11), 4.39 (br dd, J=6.4, 5.3 Hz, 1H, H-6), 4.25 (d, J=16.8 Hz, 1H, H-9a), 4.09 (d, J=16.8 Hz, 1H, H-9b), 1.41-2.32 (m, 10H, 5×CH2). ¹³C NMR (400 MHz, CDCl3) δ ppm 175.4 (C-10), 101.8 (C-7), 91.0 (C-8), 73.0 (C-6), 65.5 (C-9), 42.1 (C-5), 34.2, 29.5, 26.1 (C-2, C-3, C-4), 20.6 (C-1) ppm.

| | Raw material | | | | | | | | | |
|---|---|---|---|---|---|---|---|---|---|---|
| | | NaOMe in | | | | | | | | |
| | | MeOH | Solvent | | Temp. | Time | Product A-4 | | | Analytical data |
| # | A-3 | (30%) | DMSO | | ° C. | h | Amount | Yield | purity | TLC |
| 1 | 98 g | 0.44 L | 98 | mL | 25 | 2 | 35 g | | >95% | petroleum ether/ethyl acetate, v/v = |
| | | | | | | | | | | 1, PMA, R_{f} = 0.38 |
| 2 | 0.46 kg | 2.0 L | 0.46 | L | 25 | 2 | 0.36 kg | | >95% | petroleum ether/ethyl acetate, v/v = |
| 3 | 0.55 kg | 2.4 L | 0.55 | L | 25 | 2 | | | | 1, PMA, R_{f} = 0.38 |
| 4 | 1.8 kg | 7.8 L | 1.8 | L | 25 | 2 | 0.63 kg | 47% in 3 | >95% | petroleum ether/ethyl acetate, v/v = |
| | | | | | | | | steps | | 1, PMA, R_{f} = 0.38 |

### 2,5-dioxopyrrolidin-1-yl 2-(cyclooct-2-yn-1-yloxy)acetate (Aa)

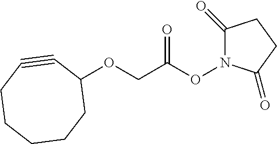 [SMILES: O=C(COC1C#CCCCCC1)ON1C(=O)CCC1=O]

To a solution of compound A-4 (COT) (0.31 kg, 2.2 mol) and HOSu (0.25 kg, 5.1 mol) in DCM (3.1 L) was added a solution of DCC (0.39 g, 1.9 mol) in DCM (0.30 L), and the mixture was stirred at 25° C. for 16 hours, which was monitored by TLC (eluting with petroleum ether/ethyl acetate, v/v=1, PMA, R_{f}=0.25 for COT was consumed and R_{f}=0.45 for compound Aa was formed). The mixture was filtered and the filtrate was concentrated in vacuo to give crude product Aa (0.53 kg, crude, containing DCU) as a white solid, which was used for the next step without further purification. ¹H NMR (400 MHz, DMSO_{d6}) δ 4.64-4.54 (m, 1H), 4.49-4.36 (m, 2H), 2.82 (br, 4H), 2.30-2.02 (m, 3H), 1.98-1.66 (m, 4H), 1.65-1.55 (m, 2H), 1.48-1.36 (m, 1H) ppm.

| | Raw material | | | Solvent | Temp | Time | Product Aa | | Product |
|---|---|---|---|---|---|---|---|---|---|
| # | A-4 | HOSu | DCC | DCM | ° C. | h | Amount | Yield | TLC |
| 1 | 30 g | 25 g | 37 g | 0.30 L + 30 mL | 0-25 | 16 | 51 g | Crude | petroleum ether/ethyl |
| | | | | | | | | | acetate, v/v = 1, PMA, R_{f} = 0.43 |
| 2 | 0.31 kg | 0.25 kg | 0.39 kg | 3.1 L + 0.3 L | 0-25 | 16 | 0.53 kg | Crude | petroleum ether/ethyl |
| | | | | | | | | | acetate, v/v = 1, PMA, R_{f} = 0.43 |

### perfluorophenyl 2-(cyclooct-2-yn-1-yloxy)acetate (COT-PFP) (Ab)

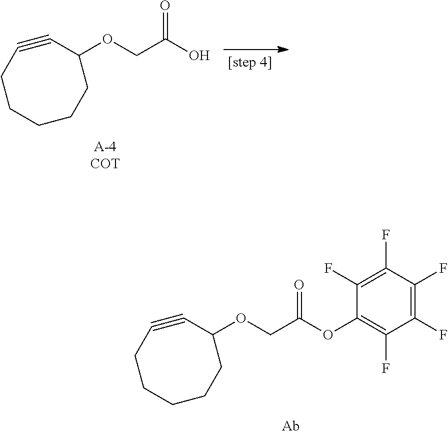 [SMILES: O=C(O)COC1C#CCCCCC1 ; O=C(COC1C#CCCCCC1)Oc1c(F)c(F)c(F)c(F)c1F]

To a solution of compound A-4 (COT) (90.0 g, 0.494 mol) and pentafluorophenol (100 g, 0.543 mol, 1.10 eq.) in DCM (0.45 L) was added a solution of DCC (112 g, 0.543 mol, 1.10 eq.) in DCM (0.45 L) within 30 minutes, and the mixture was stirred at 20° C. for 12 hours, which was monitored by TLC (eluting with petroleum ether/ethyl acetate, v/v=1, PMA, R_{f}=0.30 for COT was consumed and R_{f}=0.50 for compound Ab was formed). The mixture was filtered and the filtrate was concentrated in vacuo. The residue was purified by silica gel column chromatography (petroleum ether/ethyl acetate, v/v=50 to 10) to give crude product Ab (136 g, 375 mmol, 76% yield) as a white solid. ¹H NMR (400 MHz, CDCl₃) δ 4.59-4.54 (m, 1H), 4.50-4.43 (m, 2H), 2.35-2.14 (m, 3H), 2.14-2.04 (m, 1H), 2.02-1.79 (m, 3H), 1.68 (m, 2H), 1.58-1.46 (m, 1H) ppm.

### Example 21. Synthesis of intermediate B (Scheme 7)

The synthesis of intermediate B was reported in WO2019094395.

### 1-[2-(Cyclooct-2-yn-1-yloxy)acetamido]-3,6,9,12-tetraoxapentadecan-15-oic acid (B)

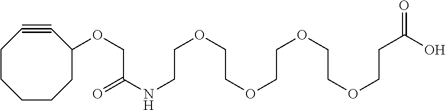 [SMILES: O=C(O)CCOCCOCCOCCOCCNC(=O)COC1C#CCCCCC1]

To a mixture of intermediate A (0.50 g, 1.8 mmol) and amido-PEG4-acid (B-1) (0.65 g, 1.8 mmol) in DMF (3 mL) was added DIPEA (1.2 g, 9.0 mmol) at room temperature. The mixture was stirred at room temperature for 30 minutes. The resulting mixture was directly purified by prep-HPLC (5-95% acetonitrile in aq. TFA (0.01%)) to give intermediate B (0.70 g, 91% yield) as light yellow oil. ESI m/z: 430 (M+H)⁺.

### Example 22. Synthesis of compound 4

Synthesis of compound 4 is described in Example 5.

### Example 23. Exemplary Synthesis of intermediate D by route a (Scheme 10)

### N-[(1S)-1-{[(1 S)-4-(carbamoylamino)-1-{[4-(hydroxymethyl)phenyl]carbamoyl}butyl]carbamoyl}-2-methylpropyl]-1-[2-(cyclooct-2-yn-1-yloxy)acetamido]-3,6,9,12-tetraoxapentadecan-15-amide (D1-2)

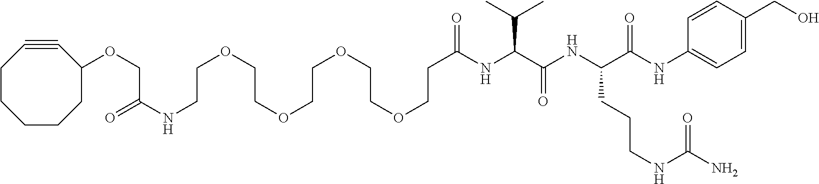 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(CO)cc1]

To a stirred solution of intermediate B (0.14 g, 0.33 mmol) in DCM (5 mL) were added HOSu (84 mg, 0.73 mmol) and EDCI (0.14 g, 0.73 mmol), and the reaction mixture was stirred at room temperature for 2 hours, which was monitored by LCMS. The mixture was diluted with DCM, washed with water (3×) and brine, dried over anhydrous sodium sulfate and concentrated in vacuo. The residue was dissolved in DMF (10 mL). To the solution were added DIPEA (0.13 g, 1.0 mmol) and vcPAB (0.13 g, 0.34 mmol), and the reaction mixture was stirred at RT for an hour. Reaction completion was monitored by LCMS. The resulting mixture was directly purified by reversed phase flash chromatography (0-80% acetonitrile in water) to give compound D1-2 (0.18 g, 70% yield) as colorless oil. ESI m/z: 791.3 (M+H)⁺. 1H NMR (400 MHz, DMSOd6) δ 9.91 (S, 1H), 8.11 (d, J=8.4 Hz, 1H), 7.89 (d, J=8.8 Hz, 1H), 7.61 (t, J=5.6 Hz, 1H), 7.55 (d, J=8.4 Hz, 2H), 7.23 (d, J=8.4 Hz, 2H), 5.98 (t, J=5.6 Hz, 1H), 5.42 (s, 2H), 5.10 (br s, 1H),), 4.43 (s, 2H), 4.39-4.37 (m, 1H), 4.30-4.21 (m, 2H), 3.87 (d, J=14.8 Hz, 1H), 3.75(d, J=14.8 101 WO 2021/174113 PCT/US2021/020074 Hz, 1H), 3.62-3.58 (m, 2H), 3.50-3.46 (m, 12H), 3.43 (t, J=6.0 Hz, 2H), 3.27-3.22 (m, 2H), 3.06-2.92 (m, 2H), 2.41-2.32 (m, 2H), 2.26-2.05 (m, 3H), 1.99-1.66 (m, 6H), 1.62-1.55 (m, 3H), 1.44-1.35 (m, 3H), 0.89 (d, J=6.8 Hz, 3H), 0.83 (d, J=6.8 Hz, 3H) ppm.

### {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl 4-nitrophenyl carbonate (Da)

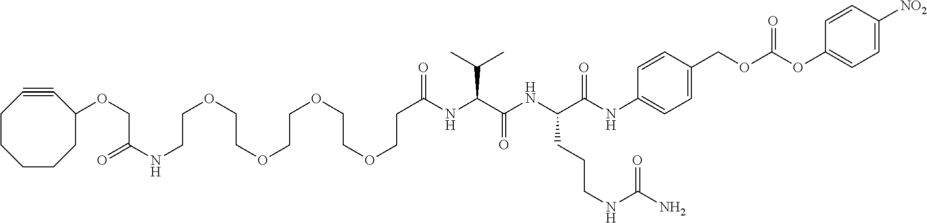 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(COC(=O)Oc2ccc([N+](=O)[O-])cc2)cc1]

A suspension of compound D1-2 (80 mg, 0.10 mmol), DMAP (12 mg, 0.10 mmol) and DIPEA (26 mg, 0.20 mmol) in dry DMF (5 ml.) was stirred at RT for 10 minutes before the addition of bis(4-nitrophenyl) carbonate (61 mg, 0.20 mmol). The reaction mixture was stirred at RT for 2 hours. Reaction completion was monitored by LCMS. The resulting mixture was directly purified by reversed phase flash chromatography (0-80% acetonitrile in water) to give intermediate Da (53 mg, 55% yield) as a white solid. ESI m/z: 956.3 (M+H)⁺.

### Example 24. Exemplary Synthesis of intermediate D by route b (Scheme 11) 2,5-dioxopyrrolidin-1-yl 1-({[(9H-fluoren-9-yl)methoxy]carbonyl}amino)-3,6,9,12-tetraoxapentadecan-15-oate (D-2) (small scale)

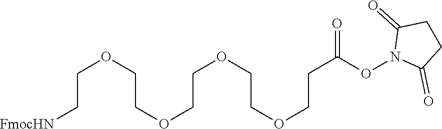

To a solution of Fmoc-N-amido-PEG4-acid (D-1) (0.74 kg, 1.5 mol, CAS: 557756-85-1) and HOSu (0.26 kg, 2.3 mol) in DCM (7.4 L) was added a solution of DCC (0.41 kg, 2.0 mol) in DCM (7.4 L) dropwise (40 mL per minute) under nitrogen atmosphere maintaining the temperature below 5° C. After the addition, the reaction mixture was stirred at 25° C. for 2 hours, which was monitored by TLC (eluting with ethyl acetate, R_{f}=0.40 for D-1 consumed and R_{f}=0.51 for D-2 formed). The resulting mixture was filtered, and the filtrate was concentrated in vacuo to give crude product D-2 (1.1 kg, crude, contaminated with DCU) as a white solid, which was used for the next step without further purification. ¹H NMR (400 MHz, CDC₃) δ 7.76 (d, J=7.5 Hz, 2H), 7.61 (d, J=7.4 Hz, 2H), 7.43-7.37 (m, 2H), 7.35-7.29 (m, 2H), 6.20 (br, 1H), 5.42 (br s, 1H), 4.41 (br d, J=6.9 Hz, 2H), 4.28-4.19 (m, 1H), 3.82 (br t, J=6.4 Hz, 2H), 3.66-3.61 (m, 12H), 3.57 (br t, J=4.9 Hz, 2H), 3.42-3.35 (m, 2H), 2.94-2.82 (m, 4H) ppm.

| | Raw material | | | Solvent | Temp. | Time | Product D-2 | | | Product |
|---|---|---|---|---|---|---|---|---|---|---|
| # | D-1 | HOSu | DCC | DMSO | ° C. | h | Amount | Yield | purity | TLC |
| 1 | 20 g | 7.1 g | 11 g | 0.40 L | 0-25 | 2 | 29 g | | Crude | ethyl acetate, |
| | | | | | | | | | | PMA, R_{f} = 0.51 |
| 2 | 0.74 kg | 0.26 kg | 0.41 kg | 15 L | 0-25 | 12 | 1.1 kg | | Crude | ethyl acetate, |
| | | | | | | | | | | PMA, R_{f} = 0.51 |

### 2,5-dioxopyrrolidin-1-yl 1-({[(9H-fluoren-9-yl)methoxy]carbonyl}amino)-3,6,9,12-tetraoxapentadecan-15-oate (D-2) (large scale)

To a solution of Fmoc-N-amido-PEG4-acid (D-1) (1.8 kg, 3.7 mol, CAS: 557756-85-1) and HOSu (0.64 kg, 5.6 mol) in DCM (9.0 L) was added a solution of DCC (0.99 kg, 4.8 mol) in DCM (9.0 L) dropwise (450 mL per minute) within 20 minutes under nitrogen atmosphere maintaining the temperature below 5° C. After the addition, the reaction mixture was stirred at 25° C. for 2 hours, which was monitored by TLC (eluting with ethyl acetate, R_{f}=0.35 for D-1 consumed and R_{f}=0.55 for D-2 formed). The resulting mixture was filtered and the filtrate was concentrated in vacuo to give crude product D-2 (2.1 kg, crude, contaminated with DCU) as light yellow oil, which was used for the next step without further purification.

### (9H-fluoren-9-yl)methyl N-(14-{[(1S)-1-{[(1S)-4-(carbamoylamino)-1-{[4-(hydroxymethyl)phenyl]carbamoyl}butyl]carbamoyl}-2-methylpropyl]carbamoyl}-3,6,9,12-tetraoxatetradecan-1-yl)carbamate (D-3)

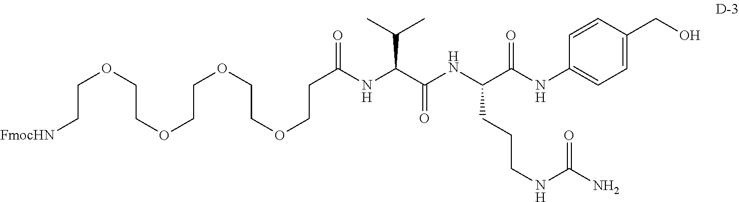

To a solution of compound D-2 (1.1 kg, crude, obtained above) in DMF (5.0 L) was added a solution of vcPAB (0.72 kg, 1.90 mol) in DMF (5.0 L) dropwise over 30 minutes, maintaining the temperature between 0° C. to 5° C. After the addition, the reaction mixture was stirred at 25° C. for 16 hours, which was monitored by TLC (eluting with DCM/methanol, v/v=8/1, PMA, R_{f}=0.57 for D-2 consumed and R_{f}=0.25 for D-3 formed). The resulting mixture was poured into a stirred aq. hydrochloride (1 M, 20 L) and the mixture was extracted with DCM/methanol (v/v=10/1, 20 L×3). The combined organic solution was washed with brine (5 L), dried over anhydrous sodium sulfate, and concentrated in vacuo. The residue was dissolved in DCM/methanol (v/v=10/1, 5 L) and the solution was dropwise added into MTBE (50 L) and stood for half an hour to precipitate a white solid, which was collected by filtration and dried in vacuum to give compound D-3 (0.94 kg, 73% yield in 2 steps from D-1) as a white solid. ¹H NMR (400 MHz, DMSO_{d6}) δ 9.92 (s, 1H), 8.87-8.50 (m, 1H), 8.11 (br d, J=7.4 Hz, 1H), 7.94-7.81 (m, 3H), 7.69 (br d, J=7.3 Hz, 2H), 7.57 (br d, J=8.4 Hz, 2H), 7.41 (br t, J=7.4 Hz, 2H), 7.38-7.28 (m, 3H), 7.24 (br d, J=8.4 Hz, 2H), 6.41-5.74 (m, 1H), 4.55-4.37 (m, 3H), 4.37-4.11 (m, 4H), 3.66-3.56 (m, 2H), 3.55-3.44 (m, 11H), 3.41 (br t, J=5.69 Hz, 2H), 3.14 (br d, J=5.50 Hz, 2H), 3.10-2.92 (m, 2H), 2.49-2.43 (m, 1H), 2.43-2.30 (m, 1H), 2.19-1.82 (m, 1H), 1.81-1.68 (m, 1H), 1.67-1.55 (m, 1H), 1.52-1.32 (m, 2H), 1.00-0.80 (m, 6H) ppm.

| | Raw material | | Solvent | Temp | Time | Product D-3 | | | Product |
|---|---|---|---|---|---|---|---|---|---|
| # | D-2 | vcPAB | DMF | ° C. | H | Amount | Yield | purity | TLC |
| 1 | 29 g | 20 g | 0.29 L | 0-25 | 16 | 25 g | | 96% | DCM/MeOH, v/v = 8/1, PMA, R_{f} = 0.25 |
| 2 | 0.15 kg | 98 g | 1.5 L | 0-25 | 16 | 0.14 kg | | 96% | DCM/MeOH, v/v = 8/1, PMA, R_{f} = 0.25 |
| 3 | 1.1 kg | 0.72 kg | 11 L | 0-25 | 16 | 0.94 kg | 73% in 2 | 95% | DCM/MeOH, v/v = 8/1, PMA, R_{f} = 0.25 |
| | | | | | | | steps | | |

### N-[(1S)-1-{[(1S)-4-(carbamoylamino)-1-{[4-(hydroxymethyl)phenyl]carbamoyl}butyl]carbamoyl}-2-methylpropyl]-1-[2-(cyclooct-2-yn-1-yloxy)acetamido]-3,6,9,12-tetraoxapentadecan-15-amide (D-4a)

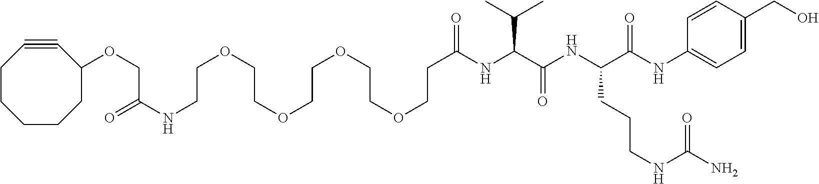 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(CO)cc1]

To a solution of Fmoc-N-amido-PEG4-vcPAB (D-3) (0.74 kg, 0.87 mol) in DMF (5.9 L) were added DBU (11 mL, 87 mmol) and triethylamine (190 mL, 1.74 mol) dropwise maintaining the temperature between 20-25° C. The reaction mixture was then stirred at 25° C. for 16 hours, and to the solution was added a solution of intermediate Aa (0.344 kg, 1.05 mol, 84.8% purity, 1.2 eq) in DMF (1.5 L). The reaction mixture was stirred for another hour and was monitored by TLC (eluting with petroleum ether/ethyl acetate, v/v=3/1, PMA, R_{f}=0.15 for D-3 consumed and R_{f}=0.40 for D-4a formed). The resulting mixture was poured into cold water (9.0 L) and washed with ethyl acetate (9.0 L). The aqueous phase was extracted with DCM (9.0 L×3) and the combined DCM solution was dried over anhydrous sodium sulfate and concentrated in vacuo. The residue was purified by prep-HPLC to give compound D-4a (0.37 kg, 52.6% yield) as a white solid. ¹H NMR (400 MHz, DMSO_{d6}) δ 9.89 (s, 1H), 8.09 (br d, J=7.4 Hz, 1H), 7.88 (br d, J=8.5 Hz, 1H), 7.68-7.45 (m, 3H), 7.24 (br d, J=7.9 Hz, 2H), 5.98 (br s, 2H), 5.09 (br t, J=5.2 Hz, 1H), 4.53-4.34 (m, 3H), 4.32-4.16 (m, 2H), 3.94-3.82 (m, 1H), 3.81-3.71 (m, 1H), 3.60 (br s, 2H), 3.56-3.25 (m, 14H), 3.30-3.20 (m, 2H), 3.07-2.89 (m, 2H), 2.50-2.33 (m, 2H), 2.29-2.04 (m, 3H), 2.02-1.82 (m, 3H), 1.81-1.65 (m, 3H), 1.64-1.50 (m, 3H), 1.49-1.29 (m, 3H), 1.00-0.80 (m, 6H) ppm.

| | Raw material | | | | | Solvent | Temp. | Time | Product D-4a | | | Product |
|---|---|---|---|---|---|---|---|---|---|---|---|---|
| # | D-3 | Int A | DBU | Et3N | | DMF | ° C. | H | Amount | Yield | purity | TLC |
| 1 | 15 g | 10 g | 0.23 mL | 4.9 | mL | 0.15 L | 0-25 | 16 + 1 | 6.0 g | | 97% | DCM/MeOH, v/v = |
| | | | | | | | | | | | | 10/1, PMA, R_{f} = 0.51 |
| 2 | 0.10 kg | 68 g | 1.5 mL | 13 | mL | 1.0 L | 0-25 | 16 + 1 | 51 g | | 97% | DDCM/MeOH, v/v = |
| | | | | | | | | | | | | 10/1, PMA, R_{f} = 0.51 |
| 3 | 0.74 kg | 0.50 kg | 11 mL | 95 | mL | 7.4 L | 0-25 | 16 + 1 | 0.32 kg | 54% | 96% | DCM/MeOH, v/v = |
| | | | | 1.7 | mol | | | | | | | 10/1, PMA, R_{f} = 0.51 |
| | | | | 87 | mmol | | | | 50 g | | 86% | DCM/MeOH, v/v = |

10/1, PMA, R_{f} = 0.51

### N-[(1S)-1-{[(1S)-4-(carbamoylamino)-1-{[4-(hydroxymethyl)phenyl]carbamoyl}butyl]carbamoyl}-2-methylpropyl]-1-[2-(cyclooct-2-yn-1-yloxy)acetamido]-3,6,9,12-tetraoxapentadecan-15-amide (D-4a) (2-step method)

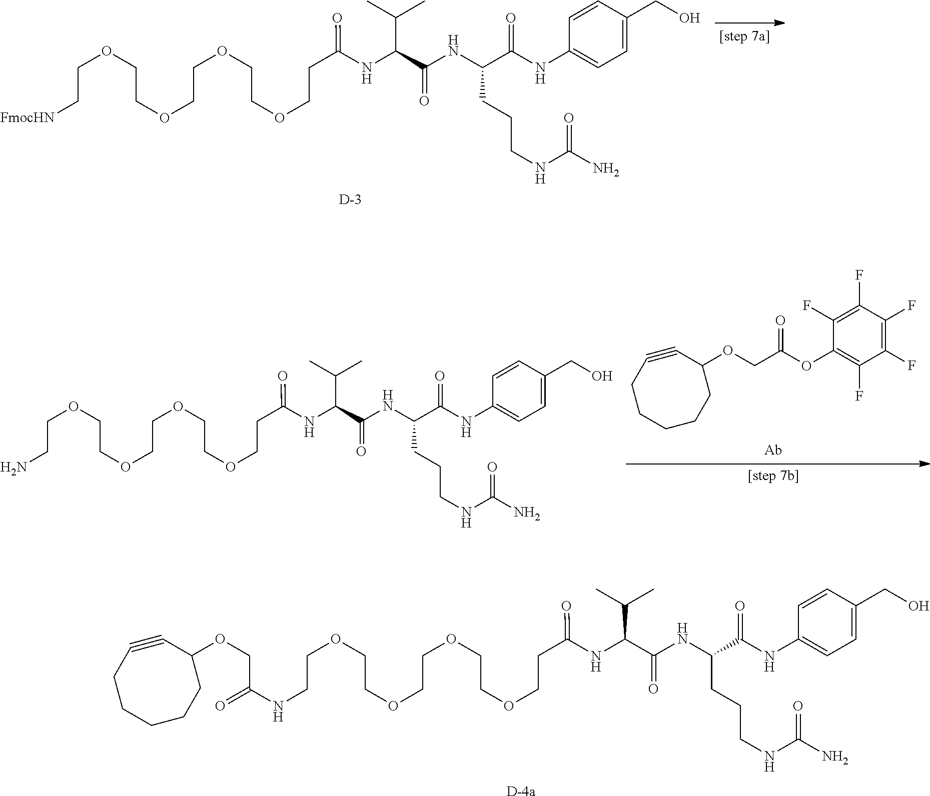

To a solution of Fmoc-N-amido-PEG4-vcPAB (0-3) (2.7 kg, 3.2 mol, 1.0 eq.) in THF (13.5 L) and water (13.5 L) was added DBU (0.70 kg, 0.63 L, 4.5 mol, 1.4 eq.) within 20 minutes maintaining the temperature between 10-20° C. The reaction mixture was then stirred at 20° C. for an hour, which was monitored by LCMS. The resulting mixture was extracted with 2-Me-THF (10 L×2) at 20° C. to remove fluorene olefin. The crude product in the aqueous solution (1.9 kg, crude) was used for the next step without further purification.

To a solution of the crude product (1.9 kg), obtained as described above, in water (10 L) was added a solution of COT-PFP (Ab) (1.1 kg, 3.1 mol) in THF (10 L) dropwise within 30 minutes maintaining the temperature below 25° C. The reaction mixture was stirred at 25° C. for an hour, which was monitored by LCMS. The resulting mixture was washed with ethyl acetate (5 L×2) to remove impurities. The aqueous phase was acidified with aq. HCl (0.5 M) until pH 3-4 at 20° C., and then extracted with DCM/MeOH (v/v=10, 10 L×3). The combined organic solution was dried over anhydrous sodium sulfate and concentrated in vacuo below 40° C. The residue was dissolved in DCM/MeOH (v/v=10, 8.0 L), and the solution was added into MTBE (150 L) dropwise for an hour. Solid was precipitated and the suspension was stood for 10 hours. The mixture was filtered. The solid was washed with MTBE (5.0 L) and dried in vacuo below 40° C. to give compound D-4a (2.0 kg, 94.8% purity, 2.4 mol, 80% yield) as a white solid. ¹H NMR (DMSO-d6, Bruker_G_400 MHz) δ 9.89 (s, 1H), 8.09 (d, J=7.6 Hz, 1H), 7.87 (d, J=8.6 Hz, 1H), 7.63-7.51 (m, 3H), 7.23 (d, J=8.4 Hz, 2H), 5.98 (br t, J=5.7 Hz, 1H), 5.40 (s, 2H), 5.09 (t, J=5.7 Hz, 1H), 4.42 (d, J=5.6 Hz, 2H), 4.38 (br d, J=5.5 Hz, 1H), 4.28 (br dd, J=5.2, 6.7 Hz, 1H), 4.23 (dd, J=6.8, 8.5 Hz, 1H), 3.92-3.84 (m, 1H), 3.78-3.72 (m, 1H), 3.59 (tt, J=3.3, 6.4 Hz, 2H), 3.52-3.47 (m, 12H), 3.42 (t, J=5.9 Hz, 2H), 3.25 (q, J=5.7 Hz, 2H), 3.06-2.90 (m, 2H), 2.48-2.44 (m, 1H), 2.42-2.34 (m, 1H), 2.28-2.13 (m, 2H), 2.01-1.82 (m, 3H), 1.81-1.64 (m, 4H), 1.64-1.52 (m, 3H), 1.50-1.31 (m, 3H), 0.85 (dd, J=6.8, 12.6 Hz, 6H) ppm.

### {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl 4-nitrophenyl carbonate (Da)

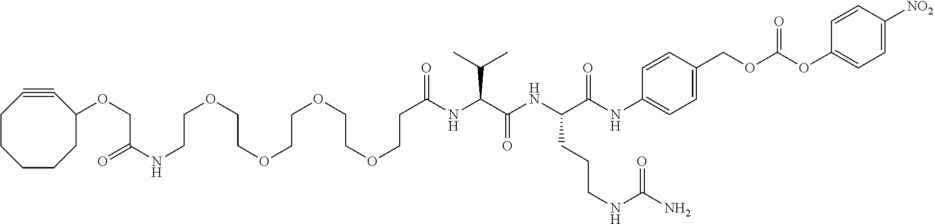 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(COC(=O)Oc2ccc([N+](=O)[O-])cc2)cc1]

To a solution of compound D-4a (0.18 kg, 0.22 mol) in DMF (1.8 L) at 22-25° C. was added DIPEA (0.12 L, 0.67 mol) dropwise during 2 minutes. The solution was cooled to 0-5° C. and to the cold solution were added bis(4-nitrophenyl) carbonate (PNP) (0.10 kg, 0.34 mol) portionwise in 15 minutes and DMAP (14 g, 110 mmol) in one portion successively. The mixture was slowly warmed and stirred at 22-25° C. for 3 hours, which was monitored by TLC (eluting with DCM/methanol, v/v=10/1, PMA, R_{f}=0.25 for D-4a consumed and R_{f}=0.45 for Da formed). The mixture was cooled to 0-5° C. and diluted with ethyl acetate (1.8 L) and poured into a stirred and cold aq. hydrochloride (0.5 M, 1.8 L). The mixture was stirred for a minute and separated. The aqueous phase was extracted with ethyl acetate (0.90 L). The combined organic solution was washed with brine, dried over anhydrous sodium sulfate, and concentrated in vacuo to around 1 L, keeping the temperature of water bath below 35° C. The residue was added dropwise to isopropyl ether (10 L) over 5 minutes and stood for half an hour. The precipitates were collected by filtration and triturated in MTBE (2 L) twice and in petroleum ether (2 L) once. The precipitates were collected and dried to give crude Da as an off-white solid, which was purified by prep-HPLC (5-95% acetonitrile in aq. formic acid (0.1%)) to give Da (80 g, 37% yield) as a white solid. ¹H NMR (400 MHz, DMSO_{d6}) δ 10.05 (s, 1H), 8.31 (d, J=8.8 Hz, 2H), 8.13 (br d, J=6.8 Hz, 1H), 7.87 (br d, J=8.5 Hz, 1H), 7.65 (d, J=8.13 Hz, 2H), 7.62-7.48 (m, 3H), 7.41 (d, J=8.3 Hz, 2H), 5.98 (br t, J=5.6 Hz, 1H), 5.42 (s, 2H), 5.24 (s, 2H), 4.44-4.34 (m, 1H), 4.32-4.19 (m, 2H), 3.91-3.83 (m, 1H), 3.79-3.72 (m, 1H), 3.64-3.56 (m, 2H), 3.56-3.45 (m, 12H), 3.45-3.40 (m, 2H), 3.30-3.20 (m, 2H), 3.09-2.89 (m, 2H), 2.48-2.44 (m, 1H), 2.42-2.31 (m, 1H), 2.27-2.02 (m, 3H), 2.02-1.82 (m, 3H), 1.81-1.66 (m, 3H), 1.65-1.52 (m, 3H), 1.49-1.31 (m, 3H), 0.90-0.80 (m, 6H) ppm.

| | Raw material | | | | | | Solvent | | Temp | Time | Product Da | | | Product |
|---|---|---|---|---|---|---|---|---|---|---|---|---|---|---|
| # | D-4a | PNP | DIPEA | | DMAP | | DMF | | ° C. | H | Amt | Yield | purity | TLC |
| 1 | 5.0 g | 2.9 g | 3.3 | mL | 77 | mg | 50 | mL | 0-25 | 4 | 22 g | | 98% | DCM/MeOH, v/v = 10/1, |
| 2 | 45 g | 26 g | 30 | mL | 3.5 | g | 0.45 | L | 0-25 | 4 | | | | PMA, R_{f} = 0.43 |
| 3 | 0.18 kg | 0.10 kg | 0.12 | L | 14 | g | 1.8 | L | 0-25 | 4 | 85 g | | 98% | DCM/MeOH, v/v = 10/1, |

PMA, R_{f} = 0.43

### {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl 4-nitrophenyl carbonate (Da) (alternative method)

To a solution of compound D-4a (0.40 kg, 94.8% purity, 0.48 mol) in DMF (4 L) was added DIPEA (186 g, 1.44 mol) dropwise during 10 minutes. To the solution were added bis(4-nitrophenyl) carbonate (PNP) (0.22 kg, 0.72 mol) portionwise (4 batches within an hour) and DMAP (5.9 g, 48 mmol) in one portion successively at 5° C. The mixture was stirred at 20° C. for 3 hours, which was monitored by LCMS.

4 batches (total 1.6 kg D-4a) of parallel reaction mixtures were combined. The mixture was diluted with ethyl acetate (20 L), and a cold aq. hydrochloride (0.5 M, 20 L) was added into the mixture. The mixture was stirred for 5 minutes and separated. The aqueous phase was extracted with ethyl acetate (10 L). The combined organic solution was washed with brine (15 L), dried over anhydrous sodium sulfate, and concentrated in vacuo to around 6 L. The residue was added dropwise to MTBE (55 L), which was slowly stirred at 20° C., during 2 hours. The precipitates were collected by filtration and triturated in MTBE (40 L) twice. The precipitates were collected and dried to give crude Da as an off-white solid, which was purified by prep-HPLC (5-95% acetonitrile in aq. formic acid (0.1%)) to give Da (1.0 kg, 98.7% purity, 38% yield) as a white solid.

RT=14.6 min in HPLC; RT=4.17 min, 3.54 min in chiral SFC; ESI m/z: 956.7 (M+H)+.

¹H NMR (DMSO-d6, Bruker_G_400 MHz) δ 10.06 (s, 1H), 8.35-8.29 (m, 2H), 8.14 (d, J=7.4 Hz, 1H), 7.88 (d, J=8.6 Hz, 1H), 7.66 (d, J=8.5 Hz, 2H), 7.62-7.54 (m, 3H), 7.42 (d, J=8.5 Hz, 2H), 5.98 (s, 1H), 5.42 (s, 2H), 5.25 (s, 2H), 4.39 (br d, J=5.8 Hz, 1H), 4.30-4.20 (m, 2H), 3.90-3.84 (m, 1H), 3.79-3.72 (m, 1H), 3.60 (dt, J=3.4, 6.3 Hz, 2H), 3.54-3.47 (m, 12H), 3.42 (t, J=5.9 Hz, 2H), 3.28-3.21 (m, 2H), 3.08-2.90 (m, 2H), 2.47 (s, 1H), 2.40 (s, 1H), 2.25-2.03 (m, 3H), 2.02-1.83 (m, 3H), 1.81-1.67 (m, 3H), 1.65-1.52 (m, 3H), 1.50-1.31 (m, 3H), 0.85 (dd, J=6.8, 13.4 Hz, 6H) ppm.[

### N-[(1S)-1-{[(1S)-4-(carbamoylamino)-1-{[4-(hydroxymethyl)phenyl]carbamoyl}butyl]carbamoyl}-2-methylpropyl]-1-{2-[4-(6-methyl-1,2,4,5-tetrazin-3-yl)phenyl]acetamido}-3,6,9,12-tetraoxapentadecan-15-amide (D-4d)

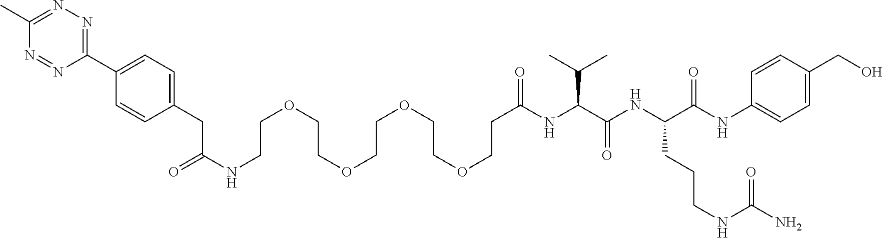 [SMILES: Cc1nnc(-c2ccc(CC(=O)NCCOCCOCCOCCOCCC(=O)N[C@H](C(=O)N[C@@H](CCCNC(N)=O)C(=O)Nc3ccc(CO)cc3)C(C)C)cc2)nn1]

To a yellow solution of intermediate Ad (0.11 g, 0.48 mmol) and DIPEA (0.19 g, 1.44 mmol) in dry DMF (2 mL) was added HATU (0.22 g, 0.57 mmol) and the mixture was stirred at room temperature for half an hour. To the stirred solution was then added amino-PEG4-vcPAB (0.27 g, 0.43 mmol). The reaction solution was stirred for 2 hours, which was monitored by LCMS. The resulting mixture was directly separated by prep-HPLC (5-95% acetonitrile in aq. TFA (0.01%)) to afford compound D-4d (0.44 g, 93% yield) as a red solid. ESI m/z: 839 (M+H)⁺.

### {4-[(2S)-5-(carbamoylamino)-2-[(2S)-3-methyl-2-(1-{2-[4-(6-methyl-1,2,4,5-tetrazin-3-yl)phenyl]acetamido}-3,6,9,12-tetraoxapentadecan-15-amido)butanamido]pentanamido]phenyl}methyl 4-nitrophenyl carbonate (Dd)

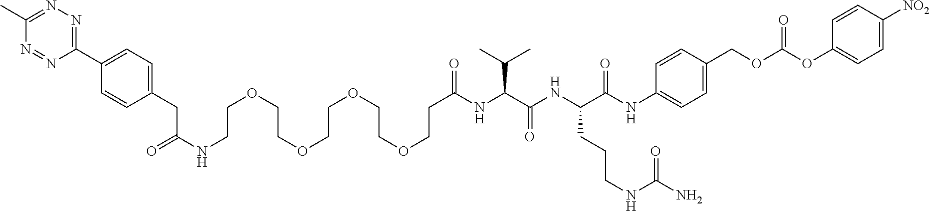 [SMILES: Cc1nnc(-c2ccc(CC(=O)NCCOCCOCCOCCOCCC(=O)N[C@H](C(=O)N[C@@H](CCCNC(N)=O)C(=O)Nc3ccc(COC(=O)Oc4ccc([N+](=O)[O-])cc4)cc3)C(C)C)cc2)nn1]

Following the similar procedure as intermediate Da except using D-4d instead of D-4a, intermediate Dd (0.24 g, 46% yield) was obtained as a red solid. ESI m/z: 1004 (M+H)+, 1026 (M+Na)⁺. ¹H NMR (400 MHz, DMSO_{d6}) δ 10.07 (s, 1H), 8.40 (d, J=8.3 Hz, 2H), 8.34-8.30 (m, 2H), 8.26 (t, J=5.6 Hz, 1H), 8.19-8.09 (m, 2H), 7.89 (d, J=8.6 Hz, 1H), 7.66 (d, J=8.6 Hz, 2H), 7.61-7.51 (m, 4H), 7.41 (d, J=8.6 Hz, 2H), 6.96-6.90 (m, 1H), 6.00 (t, J=5.6 Hz, 1H), 5.43 (br, 2H), 5.24 (br, 2H), 4.41-4.38 (m, 1H), 4.25-4.21 (m, 1H), 3.64-3.55 (m, 4H), 3.54-3.45 (m, 12H), 3.30-3.20 (m, 2H), 3.14-3.10 (m, 1H), 3.09-2.88 (m, 5H), 2.39-2.36 (m, 1H), 1.99-1.94 (m, 1H), 1.76-1.54 (m, 2H), 1.44-1.26 (m, 2H), 0.87-0.82 (m, 6H) ppm.

### Example 25. Exemplary Synthesis of intermediate E (Scheme 12)

### Synthesis of Intermediate Ea

### (2-{[({4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methoxy)carbonyl]amino}acetamido)methyl acetate (Ea)

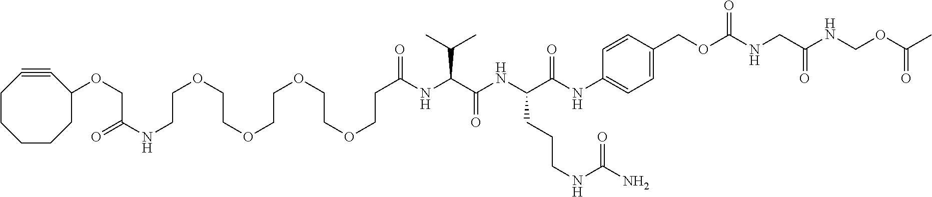 [SMILES: CC(=O)OCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1]

To a mixture of DBU (13 mg, 85 μmol) and triethylamine (0.17 g, 1.7 mmol) in DMF (10 mL) was added Fmoc-GG-OH (2a) (0.30 g, 0.85 mmol), and the mixture was stirred at 25° C. for 16 hours until Fmoc was totally removed, which was monitored by LCMS. To the solution were added intermediate D (0.81 g, 0.85 mmol), HOAt (58 mg, 0.43 mmol) and DIPEA (0.22 g, 1.7 mmol), and the reaction mixture was stirred at 25° C. for 4 hours, which was monitored by LCMS. The reaction mixture was directly purified by prep-HPLC (5-95% acetonitrile in aq. TFA (0.05%)) to give compound E-1a (0.48 g, 59% yield) as a white solid. ESI m/z: 949.5 (M+H)⁺. To a solution of compound E-1a (0.40 g, 0.42 mmol) in DMF (5 mL) were added lead(IV) acetate (0.93 g, 2.1 mmol) and acetic acid (93 mg, 1.6 mmol), and the reaction mixture was stirred at 25° C. for 16 hours under nitrogen atmosphere, which was monitored by LCMS. The resulting mixture was quenched with sat. aq. sodium bicarbonate to pH 7.0 and filtered. The filtrate was directly purified by prep-HPLC (5-95% acetonitrile in aq. TFA (0.05%)) to give intermediate Ea (0.20 g, 48% yield) as a white solid. ESI m/z: 485.3 (M+Na)⁺.

### Example 26. Exemplary Synthesis of intermediate F (Scheme 13)

### Synthesis of Intermediate F using HOAt

### 2-[(2-{[({4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methoxy)carbonyl]amino}acetamido)methoxy]ac etic acid (F)

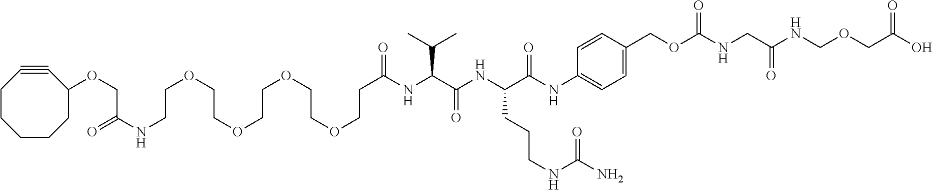 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(COC(=O)NCC(=O)NCOCC(=O)O)cc1]

To a stirred yellow solution of compound 4a (2.5 g, 6.5 mmol) in DMF (25 mL) were added DBU (0.10 g, 0.65 mmol) and triethylamine (1.3 g, 13 mmol) at 25° C., and the mixture was stirred at 25° C. for 16 hours until Fmoc was totally removed, which was monitored by LCMS. To the reaction mixture were then added a solution of intermediate D (5.0 g, 5.2 mmol) in DMF (5 mL) and HOAt (powder, 0.45 g, 3.3 mmol), and the mixture was stirred at 25° C. for 16 hours, which was monitored by LCMS. To the resulting mixture was added MTBE (2.5 L), and black oil (containing product F and DMF) was formed in the bottom. The MTBE layer (containing most Fmoc-ene and part of bases) was poured out and the layer was collected and purified directly by reversed phase flash chromatography (5-95% acetonitrile in aq. TFA (0.1%) in 60 minutes, flow rate 75 mL/min) to provide compound F (3.8 g, 62% yield, 88% purity in HPLC) as a white solid, 20 mg of which was further purified by prep-HPLC to give compound F (10 mg, >99% purity) as a white solid. ESI m/z: 979 (M+H)⁺.

### Synthesis of Intermediate F without HOAt

### 2-[(2-{[({4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methoxy)carbonyl]amino}acetamido)methoxy]ac etic acid (F)

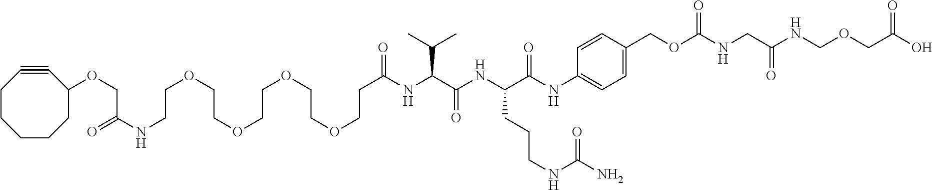 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(COC(=O)NCC(=O)NCOCC(=O)O)cc1]

To a solution of compound 4a (73 g, 0.19 mol, 1.0 eq.) in DMF (0.73 L) was added DBU (43 g, 0.28 mol, 43 mL, 1.5 eq.). The mixture was stirred at 20° C. for an hour until Fmoc was totally removed according to LCMS spectra. The resulting mixture was poured into water (0.73 L). The aqueous solution was washed with DCM (0.70 L×3) to remove the fluorene olefin, and the residual aqueous phase was used directly.

To a solution of compound Da (0.14 kg, 0.15 mmol, 0.80 eq.) in THF (0.73 L) was added the aqueous solution obtained above (0.73 L), and the mixture was stirred at 20° C. for 24 hours. The reaction mixture was then washed with ethyl acetate (0.70 L×2) and the aqueous layer was directly separated by reversed phase flash chromatography (0-100% acetonitrile in aq. formic acid (0.1%)) to give compound F (91 g, 61% yield) as a white solid. ESI m/z: 979.6 (M+H)+.

### Synthesis of Intermediate F and LP1

### Step 1

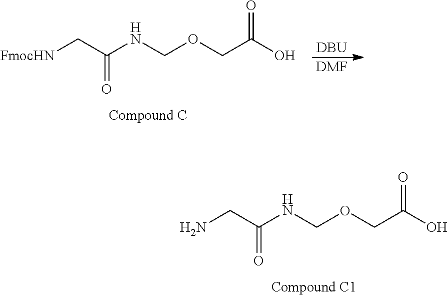

Procedures: To a solution of compound C (73 g, 189 mmol, 1.00 eq) in DMF (730 mL) was added DBU (43.3 g, 284 mmol, 42.9 mL, 1.50 eq). The mixture was stirred at 20° C. for 1 hr. LCMS (EW46238-12-P1A1) showed the compound C was consumed completely. The reaction mixture was poured into H₂O (730 mL), then washed with DCM (700 mL*3) to remove the Fluorene olefin, the aqueous phase was used for the next step directly.

### Step 2

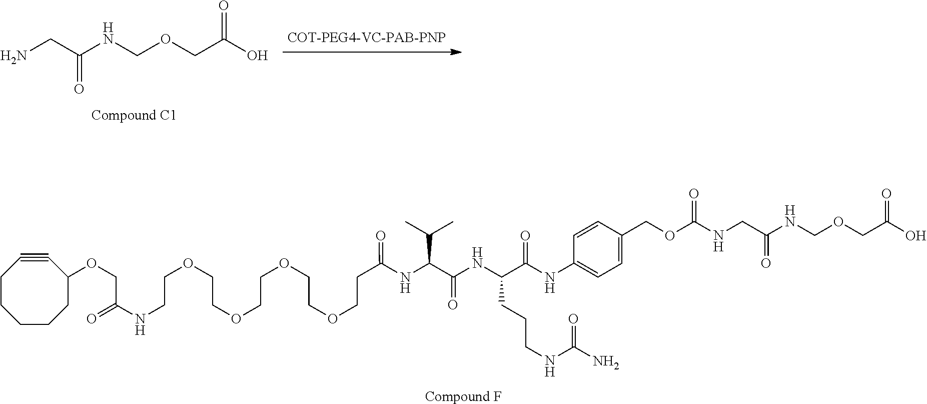 [SMILES: NCC(=O)NCOCC(=O)O ; CC(C)[C@H](NC(=O)CCOCCOCCOCCOCCNC(=O)COC1C#CCCCCC1)C(=O)N[C@@H](CCCNC(N)=O)C(=O)Nc1ccc(COC(=O)NCC(=O)NCOCC(=O)O)cc1 ; "Compound F"]

Procedures: To a solution of COT-PEG4-VC-PAB-PNP (144 g, 151 mmol, 0.80 eq) in THF (730 mL) was added and compound C1 (30.7 g, 189 mmol, 1.00 eq) in H₂O (730 mL). The mixture was stirred at 20° C. for 24 hrs. The reaction mixture was washed with EtOAc (700 mL*2). The crude product in the water layer was purified directly by reversed-phase HPLC or flash reverse phase chromatography using water/acetonitrile (0.1% TFA) mobile phases to afford Compound F with a purity of 98%. ESI m/z MH+ 979.6; Proton NMR: HNMR (DMSO-d6, Bruker_G_400 MHz) δ: 10.00 (s, 1H), 8.72 (br t, J=6.7 Hz, 1H), 8.12 (d, J=7.4 Hz, 1H), 7.88 (d, J=8.6 Hz, 1H), 7.60 (br d, J=8.4 Hz, 3H), 7.45 (t, J=6.1 Hz, 1H), 7.30 (br d, J=8.5 Hz, 2H), 6.14-5.90 (m, 1H), 4.97 (s, 2H), 4.61 (d, J=6.8 Hz, 2H), 4.45-4.35 (m, 2H), 4.31-4.21 (m, 3H), 3.99 (s, 2H), 3.91-3.85 (m, 1H), 3.79-3.73 (m, 1H), 3.65-3.56 (m, 4H), 3.52-3.47 (m, 12H), 3.46-3.41 (m, 2H), 3.25 (q, J=5.7 Hz, 2H), 3.09-2.89 (m, 2H), 2.50-2.44 (m, 1H), 2.42-2.34 (m, 1H), 2.28-2.03 (m, 3H), 2.02-1.81 (m, 3H), 1.80-1.65 (m, 3H), 1.64-1.51 (m, 3H), 1.47-1.30 (m, 3H), 0.85 (dd, J=6.8, 13.1 Hz, 6H). Intermediate F is then used to prepare LP1 by following Scheme 4A described above and further detailed showing below.

### Step 3

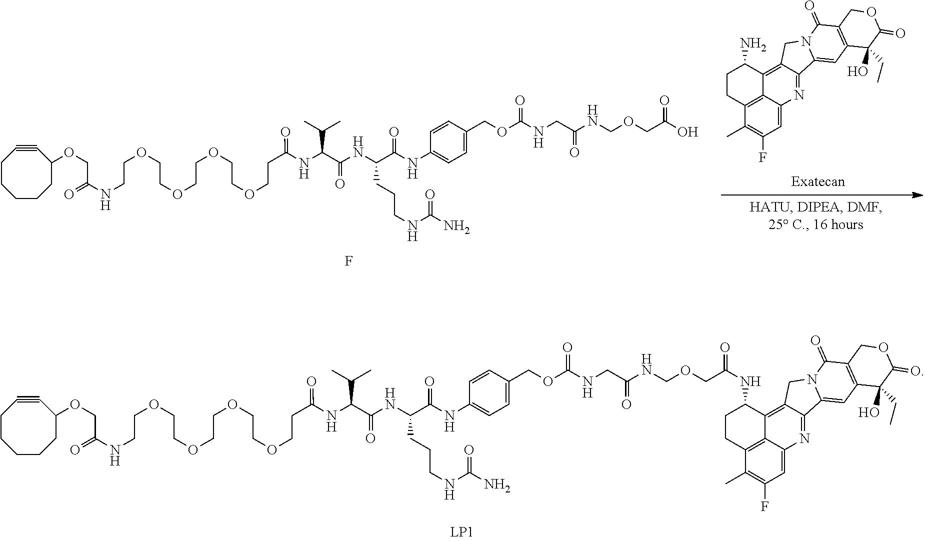

### Conjugations

### Example 27. Exemplary Conjugates

Generic procedures for making site-specific conjugates

Aglycosylated human antibody IgG (IgG1, IgG4, etc.) containing an N297Q or N297D mutation were used in ADC conjugations. Two Approaches (I & II) were conducted via two-step process (Scheme 19), and the conjugation results with the MS-DAR values are summarized in Table 10.

Step 1: site-specific conjugation of Handle-functionalized amine with an Antibody generated a drug conjugate containing 2, 4 or 8 handles per antibody.

Aglycosylated human antibody IgG containing an N297Q mutation or N297D mutation in BupH buffer (pH7.4) was mixed with >=100 molar equivalents of non-branched Handle-amine (AL) or branched Handle-amine (BL). The resulting solution was mixed with transglutaminase (25U/mL; 1 U mTG per mg of antibody, Zedira, Darmstadt, Germany; or 10 U/mL; 5.5U MTG per mg of antibody, Modernist Pantry-ACTIVA TI contains Maltodextrin from Ajinomoto, Japan) resulting in a final concentration of the antibody at 0.5-20 mg/mL. The reaction mixture was incubated at 25-37° C. for 24 hours while gently shaking while monitored by ESI-MS. Upon the completion, the excess amine and mTG were removed by size exclusion chromatography (SEC) or protein A column chromatography. The conjugate was characterized by UV-Vis, SEC and ESI-MS.

TABLE 9
List of Handles

| Cpd# | name | Structures | CAS | Availability |
|---|---|---|---|---|
| AL1 | TCO—(CH₂)₃NH₂ | 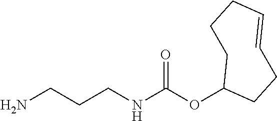 [SMILES: NCCCNC(=O)OC1CC/C=C/CCC1] | 134179- 38-7 | Commercial |
| AL2a, n = 3 AL2b, n = 4 AL2c, n = 5 AL2d, n = 8 | TCO— PEG₃—NH₂ (CP2414) TCO— PEG₄—NH₂ TCO— PEG₅—NH₂ (CP2415) TCO— PEG₈—NH₂ | 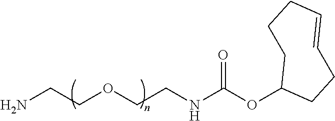 | 1800507- 93-0 2243569- 24-4 2755509- 50-1 2353409- 92-2 | Commercial |
| AL3a, n = 3 AL3b, n = 4 | BCN— PEG₃—NH₂ (CP2373) BCN— PEG₄—NH₂ | 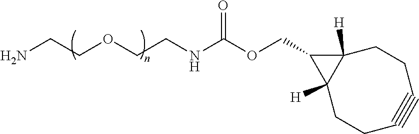 | 1883512- 27-3 1898221- 77-6 | Commercial |
| AL4a, n = 0 AL4b, n = 1 AL4c, n = 3 | COT— (CH₂)₂—NH₂ COT— PEG—NH₂ COT— PEG₃—NH₂ (CP2345), | 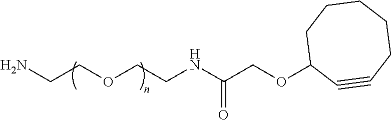 | 1809789- 76-1 2401876- 51-3 New | WO2022015656 WO2019240219 |
| AL5 | NBE— OCH₂CH₂—NH₂ | 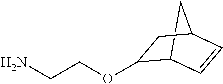 [SMILES: NCCOC1CC2C=CC1C2] | 92877- 26-4 | Commercial |
| AL6 | TzH—NH₂ | 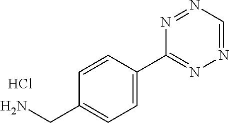 [SMILES: Cl ; NCc1ccc(-c2nncnn2)cc1] | 1416711- 59-5 | Commercial |
| AL7 | Tz—NH₂ | 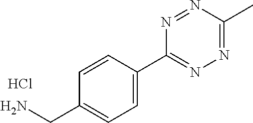 | 1596117- 29-1 | Commercial |
| AL8 | TzO— (CH₂)₃—NH₂ | 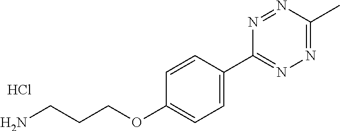 [SMILES: Cl ; Cc1nnc(-c2ccc(OCCCN)cc2)nn1] | 1802908- 03-7 | Commercial |
| AL9 | TzO— PEG₃—NH₂ | 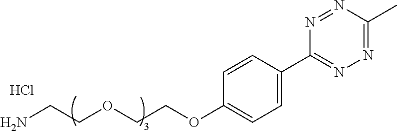 | 2245150- 18-7 | Commercial |
| AL10a, n = 4 AL10b, n = 8 AL10c, n = 12 | Tz—NHCO— PEG₄—NH₂ Tz—NHCO— PEG₈—NH₂ Tz—NHCO— PEG₁₂—NH₂ | 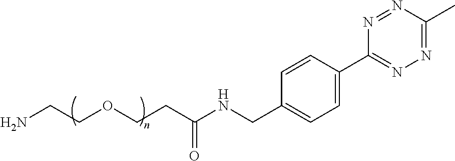 | 2055646- 21-2 2143958- 58-9 New | Commercial Commercial |
| AL11a, n = 3 AL11b, n = 4 AL11c, n = 6 AL11d, n = 8 | Az— PEG₃—NH₂ Az— PEG₄—NH₂ Az— PEG₆—NH₂ Az— PEG₈—NH₂ | 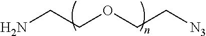 | 134179- 38-7 951671- 92-4 957486- 82-7 857891- 82-8 | Commercial |
| BL1 | (Tz—NH)₂— BR₂—PEG₃— NH₂. | 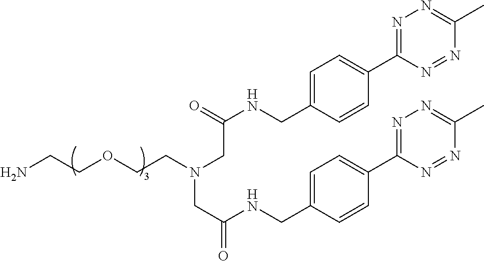 | NEW | See Scheme 3 |
| BL2 | (Tz—NH) (Az—PEG₂)— BR₂—PEG₃—NH₂ | 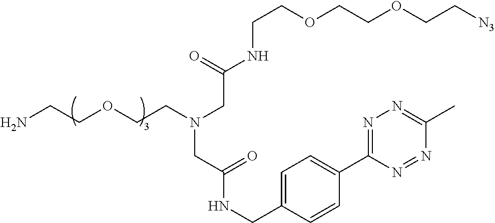 | NEW | See Scheme 4 |
| BL3a, n = 0 BL3b, n = 3 | (Az— CH₂CH₂)₂—BR₂— PEG₃—NH₂ (Az— PEG₃)₂—BR₂— PEG₃—NH₂ | 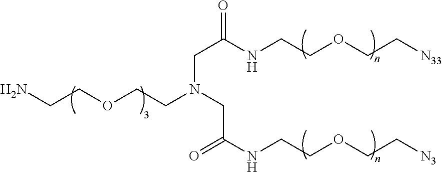 | 2758876- 09-2 2758876- 11-6 | WO2022015656 |

| Code | Structure | MF | MW |
|---|---|---|---|
| AL2a | 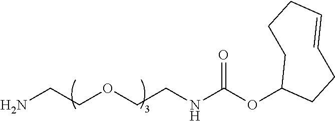 | C₁₇H₃₂N₂O₅ | 344.5 |
| AL3a | 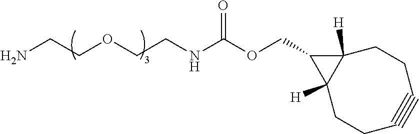 | C₁₉H₃₂N₂O₅ | 368.5 |
| AL4c | 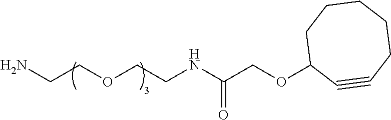 | C₁₈H₃₂N₂O₅ | 356.5 |
| AL7 | 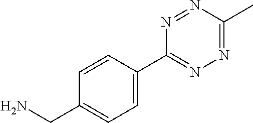 | C₁₀H₁₁N₅ | 201.2 |
| AL6 | 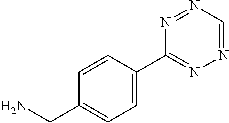 [SMILES: NCc1ccc(-c2nncnn2)cc1] | C₉H₉N₅ | 187.2 |
| AL11a | NH₂—(CH₂CH₂O)₃—N₃ | C₈H₁₈N₄O₃ | 218 |
| BL1 | 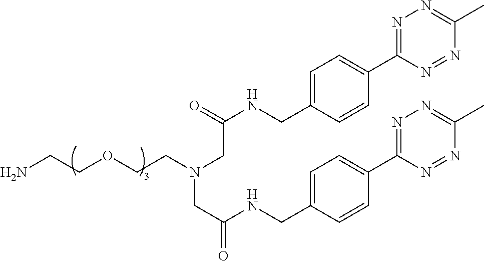 | C₃₂H₄₂N₁₂O₅ | 674.8 |
| BL2 | 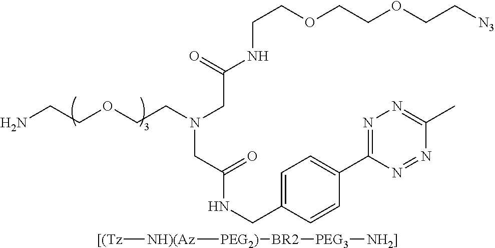 | C₂₈H₄₅N₁₁O₇ | 647.7 |
| BL3b | 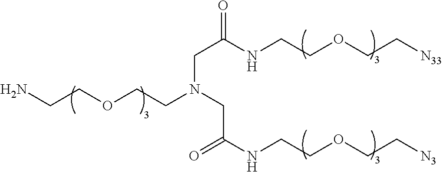 | C₂₈H₅₆N₁₀O₁₁ | 708.8 |

Step 2: click reactions between Handle-functionalized antibodies and a Linker-Payload in Table 3 to generate the site-specific ADCs.

The Handle-functionalized antibody (Ab-(AL)ₙ or Ab-(BL)ₙ, 1-20 mg/mL) in PBS (pH7.4) was incubated with >2-10 molar equivalents of a linker-payload (LP) dissolved in an organic solvent such as DMSO or DMA (10 mg/mL) to have the reaction mixture containing 5-15% organic solvent (v/v), at 25-37° C. for 1-48 hours while gently shaking. The reaction was monitored by ESI-MS. Upon completion, the excess amount of LP and organic solvent were removed by desalting column with BupH (pH 7.4) and protein aggregates (if any) were removed by size exclusion chromatography (SEC). The purified conjugate, Ab-(AL-LP)ₙ ADC or Ab-(BL-LP)₄ ADC, was concentrated, sterile filtered and characterized by UV-Vis, SEC and ESI-MS. Conjugates monomer purity was >95% by SEC.

T-DXd was conjugated using our in-house Trastuzumab; both T-DXd and Isotype Ab-DXd ADCs were conjugated with Daiichi's maleimide-tetrapeptide GGFG-linker (“GGFG” disclosed as SEQ ID NO: 2142) DXd, Antibody interchain cysteine conjugations with the maleimide linker payload were accomplished using conventional procedures. Under physiological conditions, the imide ring of the linker connecting an antibody and the payload exists as an equilibrium between an opened ring and a closed five-membered imide ring. Those ADC species with an opened ring and a closed five-membered imide ring show the same or comparable activities in our research.

All ADCs were purified by SEC using an ÄKTA instrument from Cytiva, using a 16/600 Superdex® 200 column, eluting with DPBS, at a flow rate of 1.5 mL/min at pH 7.4. The DAR values of the ADCs were measured by ESI-MS. A mass increase of 4×LP from Ab-[AL]₄ was observed, correlating to 4DAR ADC. An additional mass increase of 7 to 8×LP from Ab-[BL]₄ was observed, indicating a 7 to 8DAR ADC.

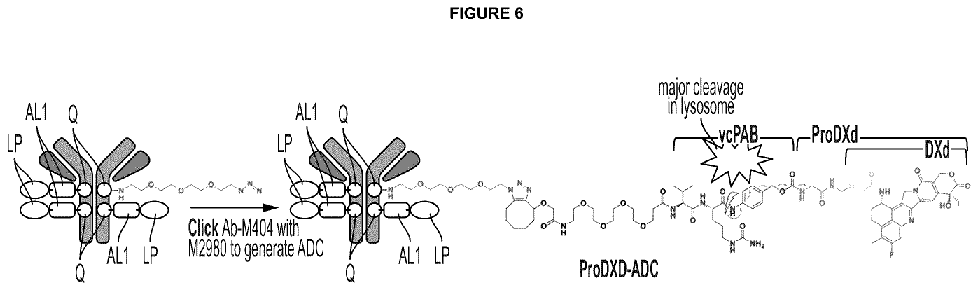

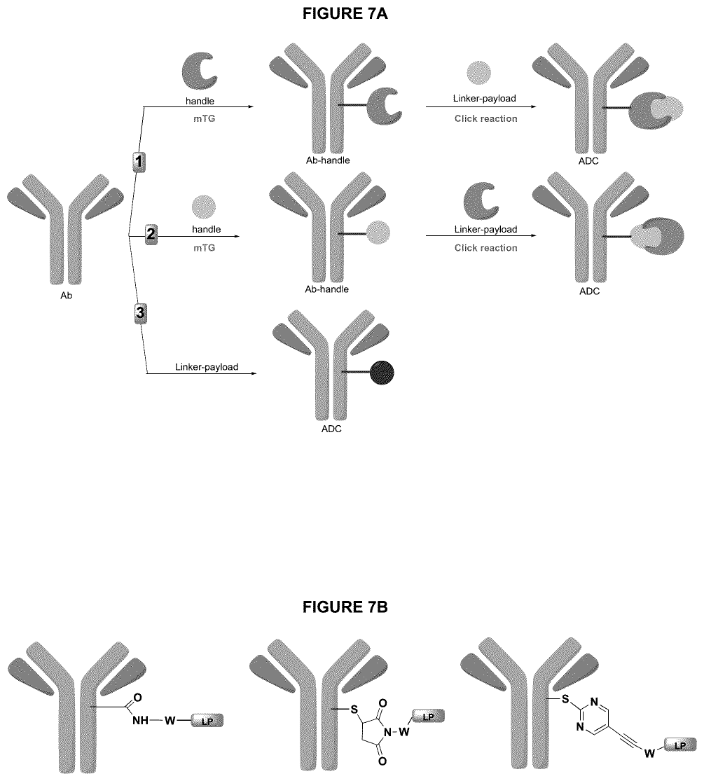

A detailed conjugation procedure is described in FIG. 6.

A representative 4DAR ADC from Approach I is exemplified following. The aglycosylated anti-Her2 human IgG antibody containing an N297Q mutation was mixed with >200 molar equivalents of a azido-dPEG3-amine (AL1, MW 708.41 g/moL). The resulting solution was mixed with microbial transglutaminase (10U/mL; 5,5U mTG per mg of antibody, Modernist Pantry-ACTIVA TI contains Maltodextrin from Ajinomoto, Japan) resulting in a final concentration of the antibody at 5 mg/mL. The reaction mixture was incubated at 37° C. for 24 hours while gently shaking while monitored by ESI-MS. Upon the completion, the excess amine and mTG were removed by size exclusion chromatography (SEC). The conjugate was characterized by UV-Vis, SEC and ESI-MS. The azido linkers attached antibody resulted in a 808 Da mass increase compared to mAb, indicating 4 AL1 was conjugated to the antibody (Ab-(AL1)₄) with 4 azido handles. The site-specific antibody azido conjugate (2.1 mg/mL) in PBS (pH7.4) was mixed with 7 molar equivalents of linker-payload (LP1) in 2 mM of DMSO to have the reaction mixture containing 5% organic solvent (v/v), and the solution was set at 32° C. for 36 hours while gently shaking. The reaction was monitored by ESI-MS. Upon completion, the excess amount of linker-payload and protein aggregates were removed by size exclusion chromatography (SEC). The purified conjugate was concentrated, sterile filtered and characterized by UV-Vis, SEC and ESI-MS. Conjugates monomer purity was 99.8% by SEC. The drug attached antibody resulting in a 6388 Da mass increase for the DAR4 conjugate. Conjugates monomer purity was >99% by SEC.

Exemplary non-limiting embodiments of antibody-linkers and ADCs of the present disclosure are shown in Table 10, below. NTC stands for non-targeting control antibody.

TABLE 10
Listing of Antibodies, Antibody-Handles, and ADCs According to the Present Disclosure

| # | ADC# | DAR | Structures |
|---|---|---|---|
| 1 | aHer2 | | |
| | Ab | | |
| 2 | FelD1 | | |
| | Ab | | |
| 3 | aHer2- (AL11a)n | 4 | 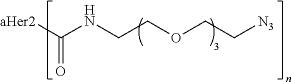 |
| 4 | FelD1- (AL11a)n | 4 | 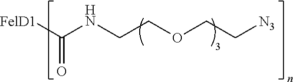 |
| 5 | aHer2- (AL2a)n | 4 | 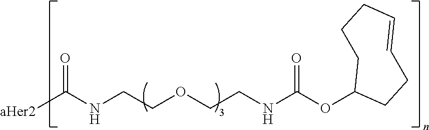 |
| 6 | FelD1- (AL2a)n | 4 | 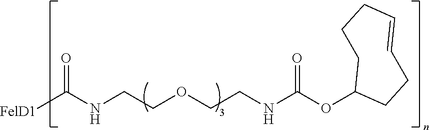 |
| 7 | aHer2- (AL3a)n | 3.94 | 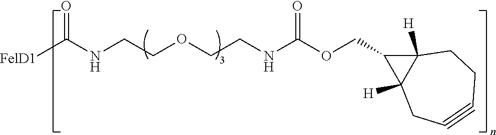 |
| 8 | FelD1- (AL3a)n | 4 | 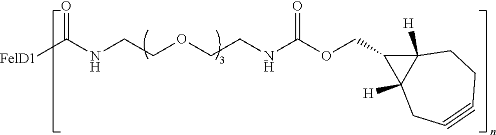 |
| 9 | aHER2- (AL7)n | 4 | 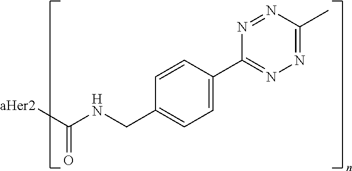 |
| 10 | FelD1- (AL7)n | 4 | 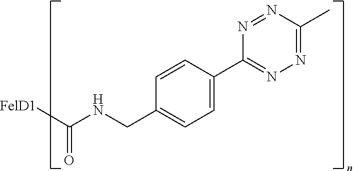 |
| 11 | aHER2- (AL6)n | 3.6 | 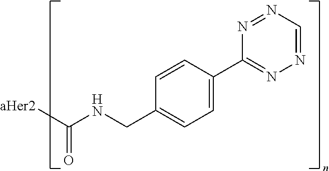 |
| 12 | aHer2- (BL1)n | 3.9 | 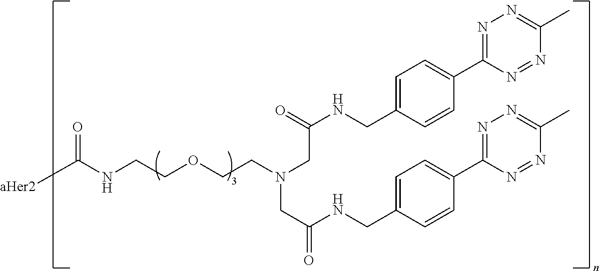 |
| 13 | FelD1- (BL1)n | 3.9 | 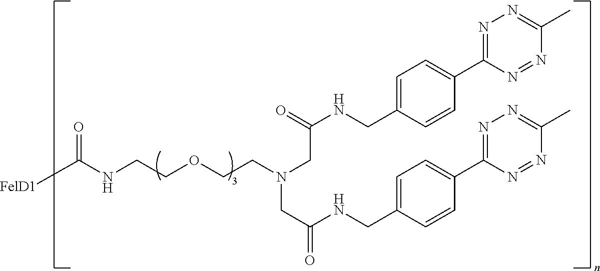 |
| 14 | aHer2- (BL3b)n | 3.8 | 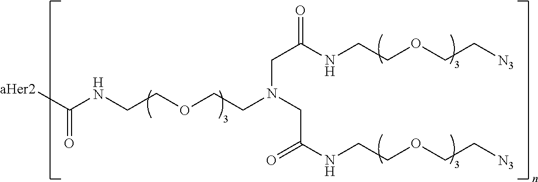 |
| 15 | FelD1- (BL3b)n | 3.8 | 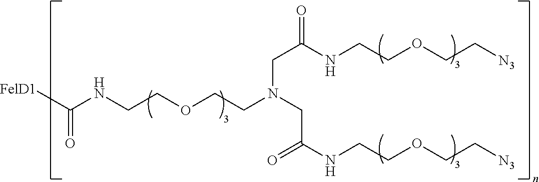 |
| 16 | aHer2- (AL6- LP1)n | 3.6 | 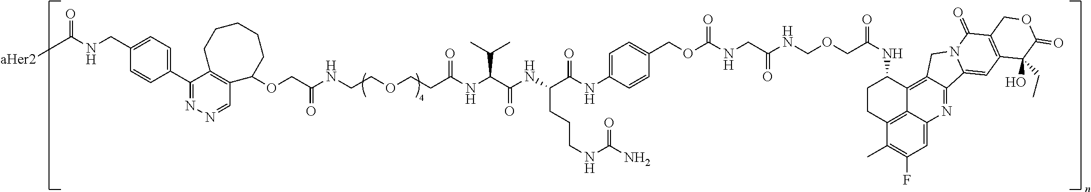 |
| 17 | aHer2- (AL6- LP13D)n | 6.2 | 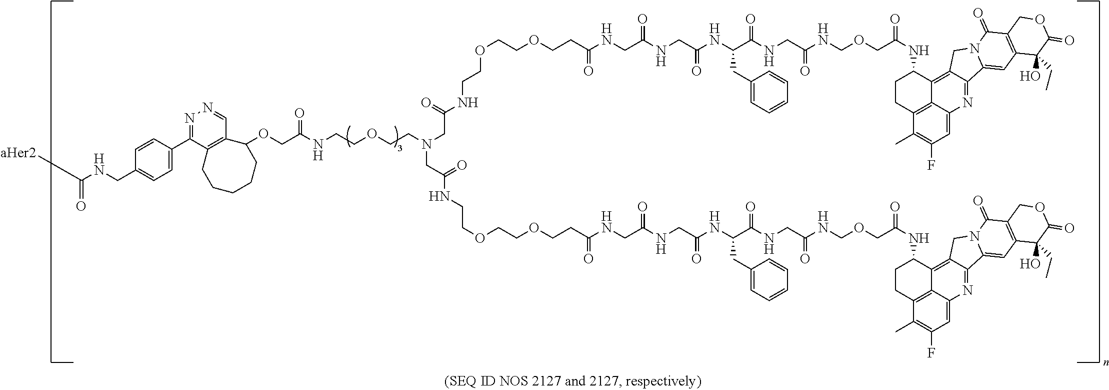 |
| 18 | aHer2- (AL6- LP13)n | 6.5 | 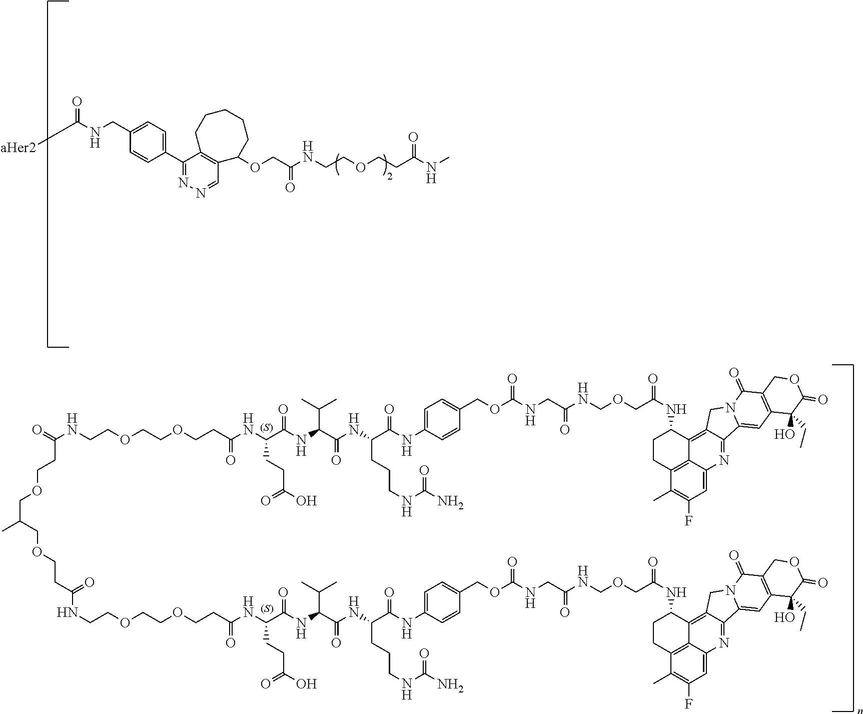 |
| 19 | aHer2- (AL7- LP1)n | 3.7 | 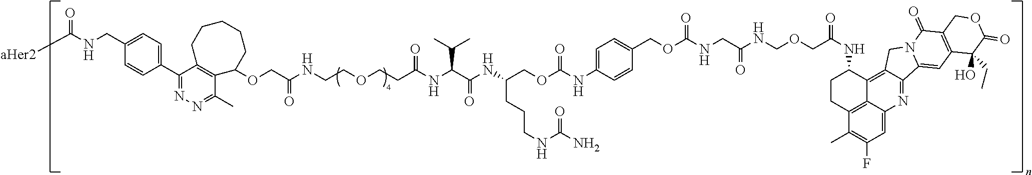 |
| 20 | FelD1- (AL7- LP1)n | 3.9 | 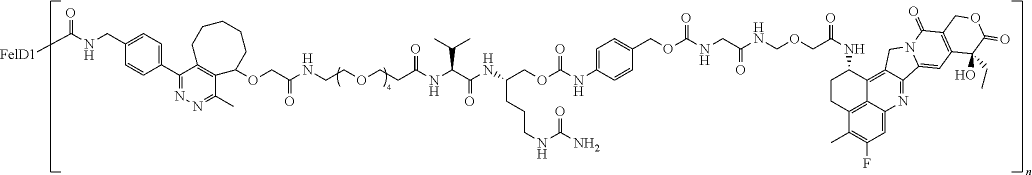 |
| 21 | aHer2- (AL7- LP13)n | 7.3 | 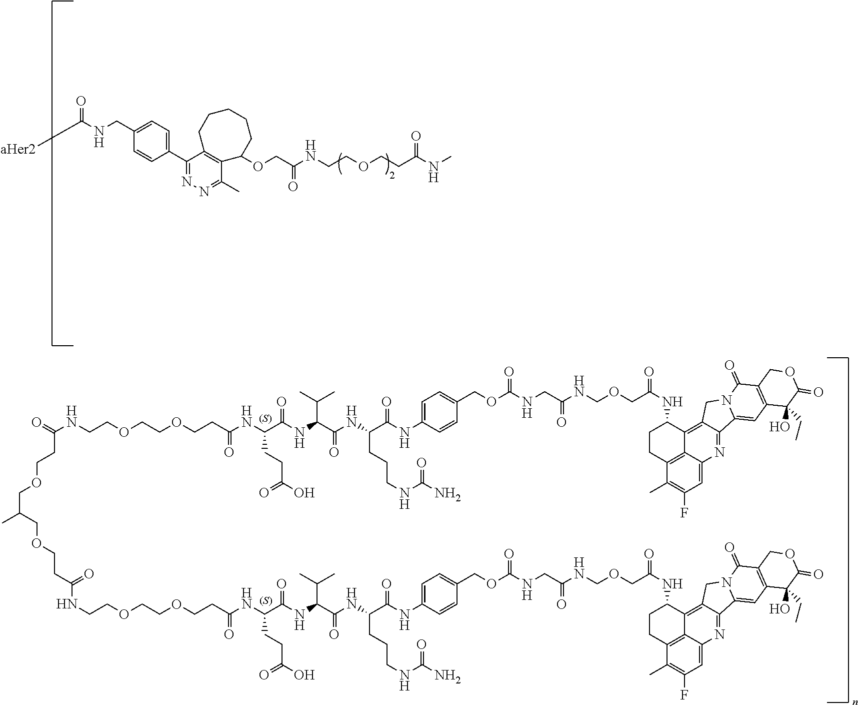 |
| 22 | FelD1- (AL7- LP13)n | 7.3 | 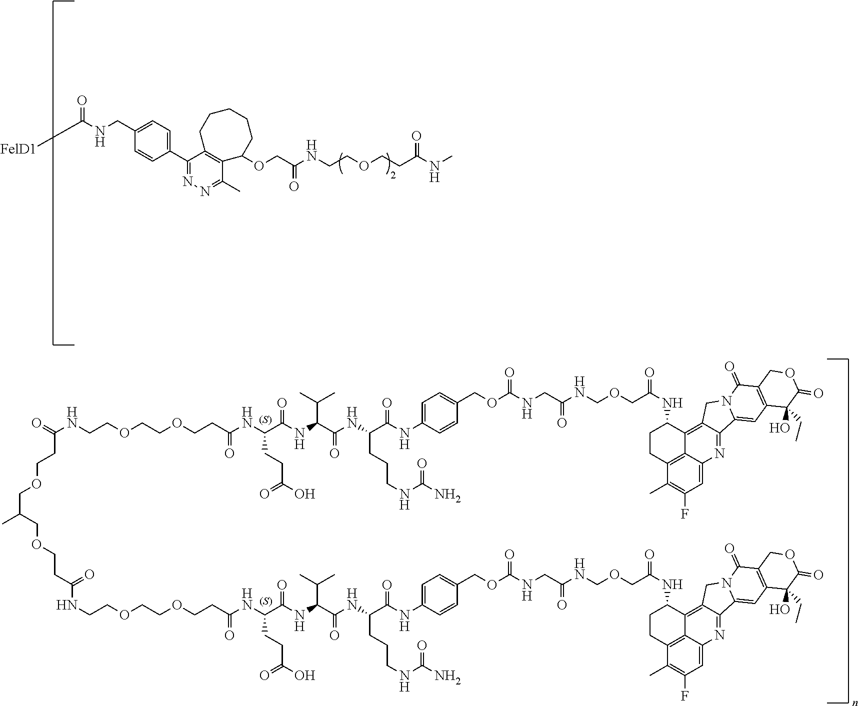 |
| 23 | aHer2- (AL7- LP2)n | 3.83 | 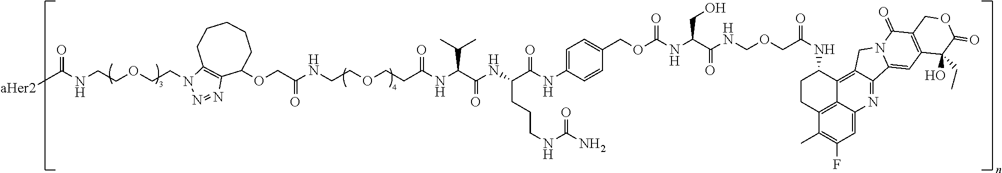 |
| 24 | FelD1- (AL7- LP2)n | 3.80 | 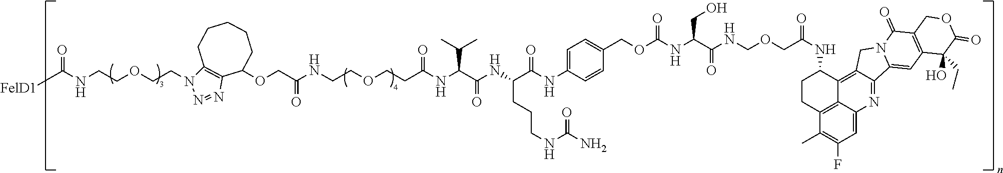 |
| 25 | aHer2- (AL7- LP3)n | 3.88 | 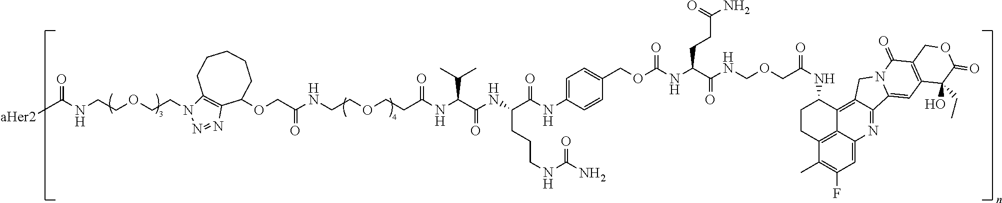 |
| 26 | FelD1- (AL7- LP3)n | 3.82 | 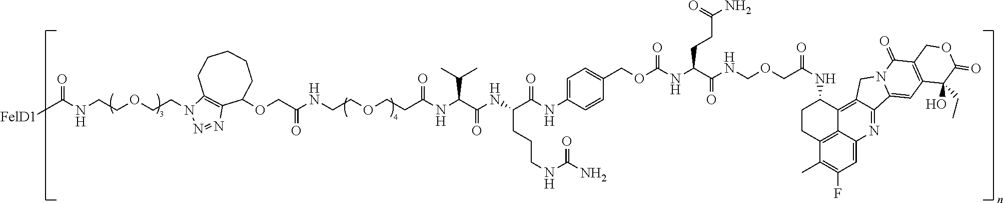 |
| 27 | aHer2- (AL7- LP4)n | 3.84 | 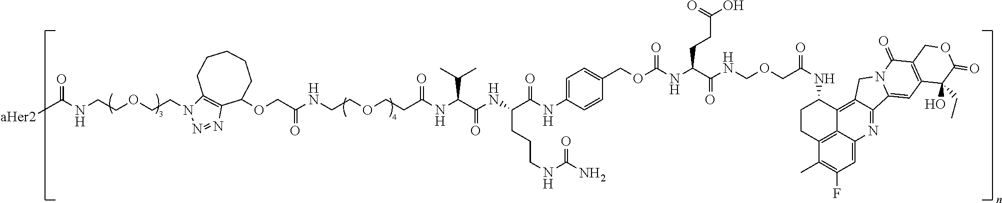 |
| 28 | FelD1- (AL7- LP4)n | 3.82 | 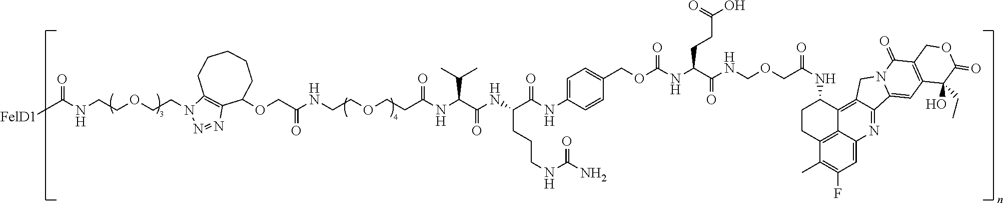 |
| 29 | aHer2- (AL7- LP5)n | 3.80 | 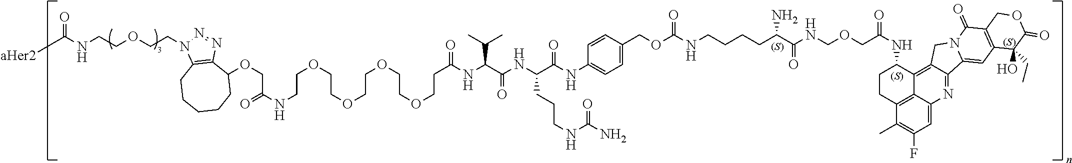 |
| 30 | FelD1- (AL7- LP5)n | 3.69 | 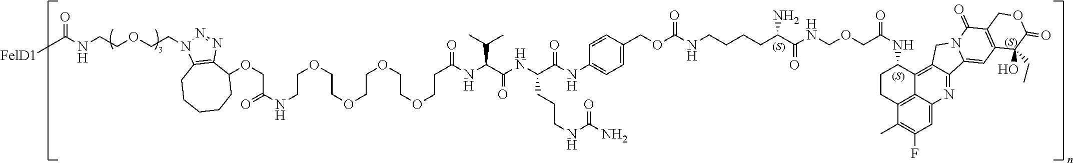 |
| 31 | aHer2- (AL7- LP7)n | 3.70 | 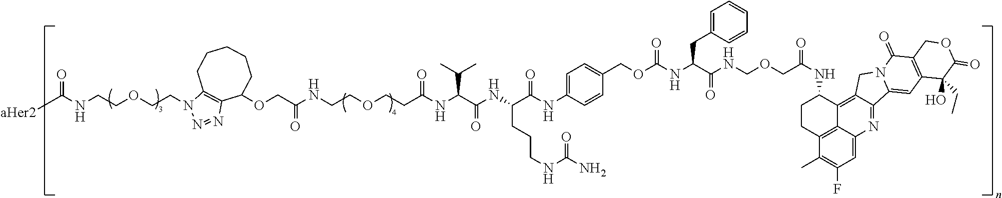 |
| 32 | FelD1- (AL7- LP7)n | 3.68 | 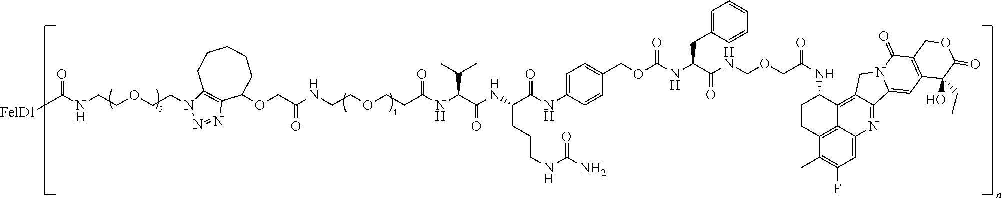 |
| 33 | aHer2- (AL7- LP9)n | 3.81 | 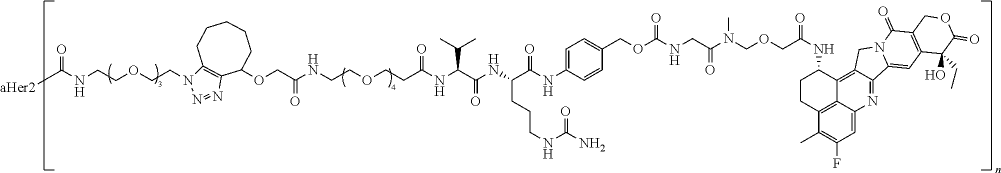 |
| 34 | FelD1- (AL7- LP9)n | 3.89 | 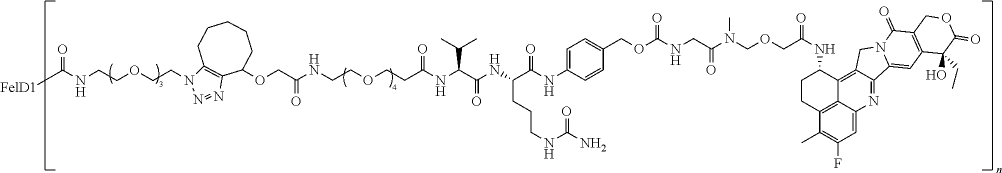 |
| 35 | aHer2- (AL7- LP12)n | 3.82 | 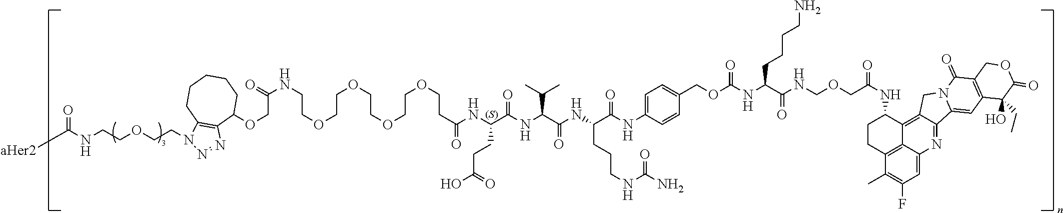 |
| 36 | FelD1- (AL7- LP12)n | 3.83 | 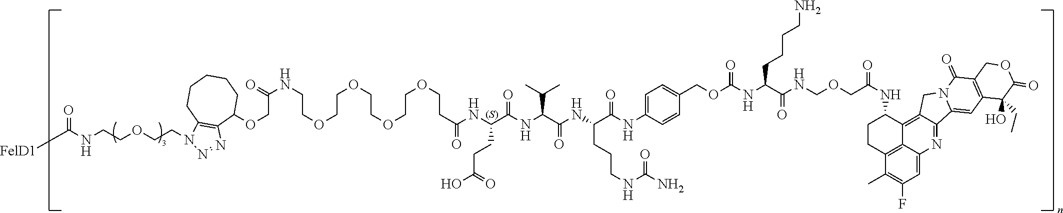 |
| 37 | aHer2- (AL7- LP5D)n | 3.89 | 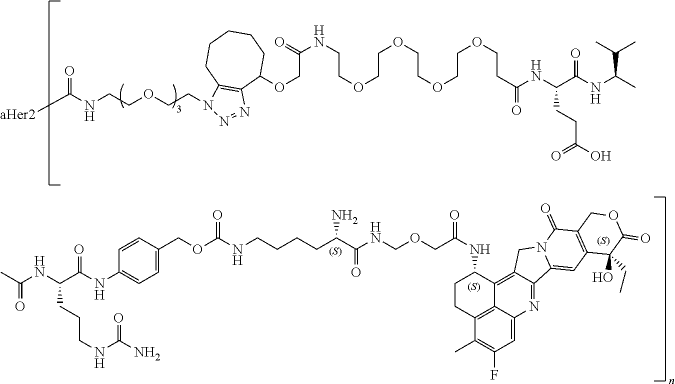 |
| 38 | aHer2- (AL7- LP5D)n | 3.90 | 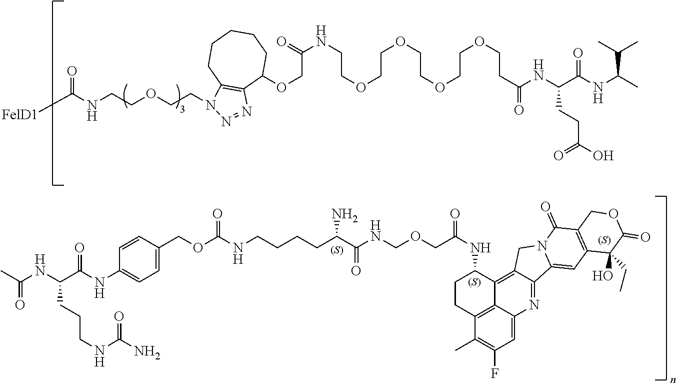 |
| 39 | aHer2- (AL4c- LP1B)n | 3.49 | 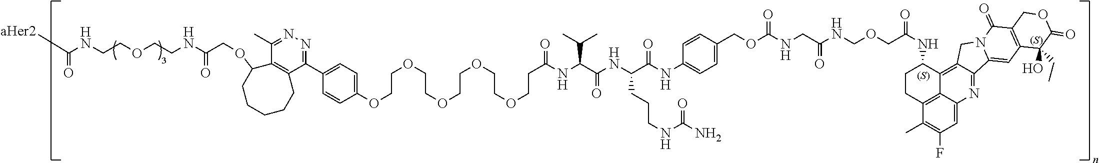 |
| 40 | FelD1- (AL4c- LP1B)n | 3.45 | 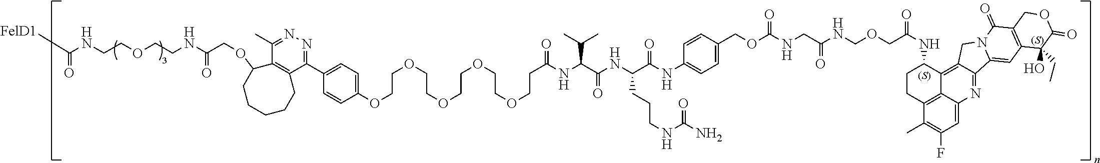 |
| 41 | aHer2- (AL4c- LP13C)n | 7.07 | 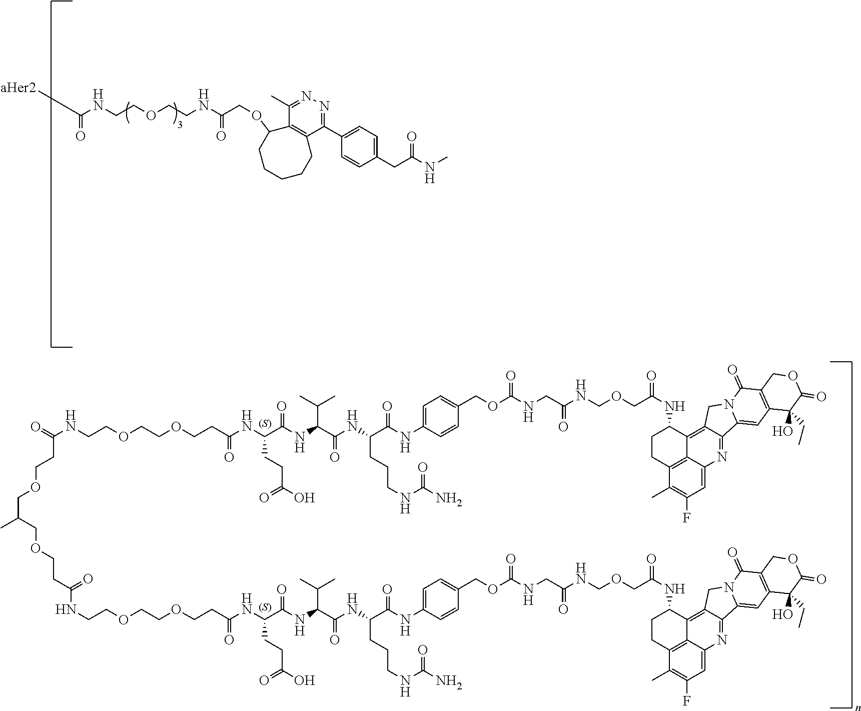 |
| 42 | aHer2- (AL4c- LP13C)n | 7.11 | 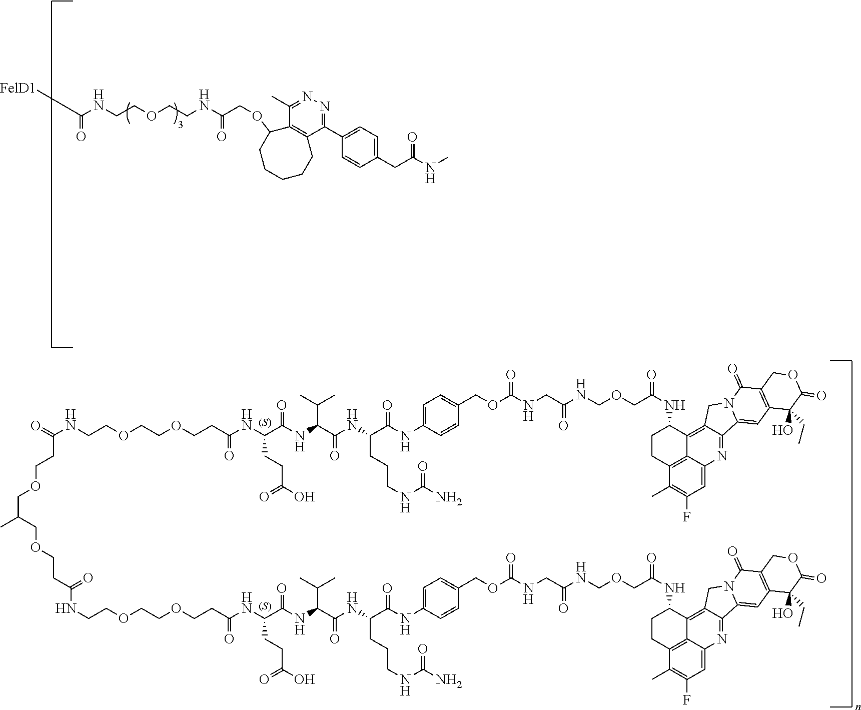 |
| 43 | aHer2- (AL11a- LP1)n | 4 | 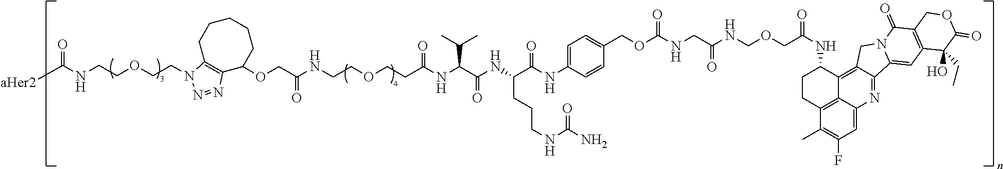 |
| 44 | FelD1- (AL11a- LP1)n | 4 | 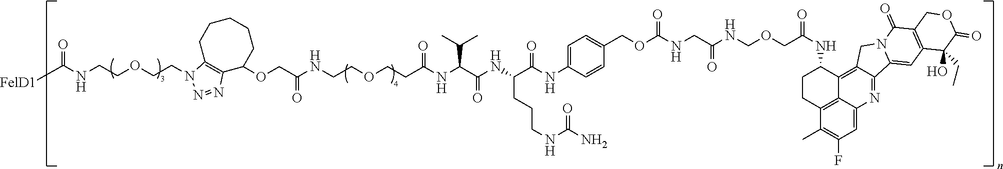 |
| 45 | aHer2- (AL11a- LP13)n | 7.8 | 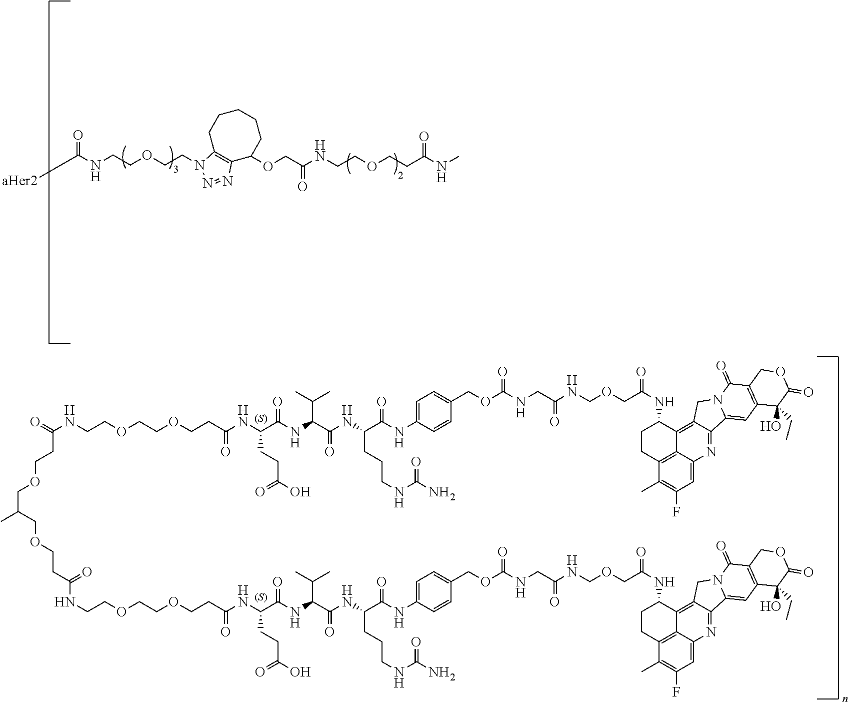 |
| 46 | FelD1- (AL11a- LP13)n | 7.3 | 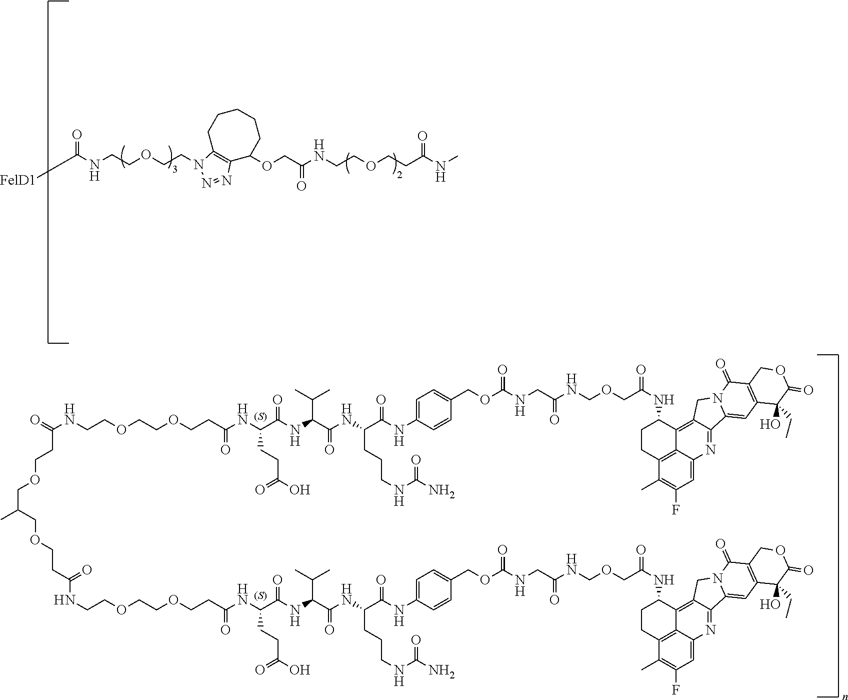 |
| 47 | aHer2- (AL11a- LP15)n | 7.6 | 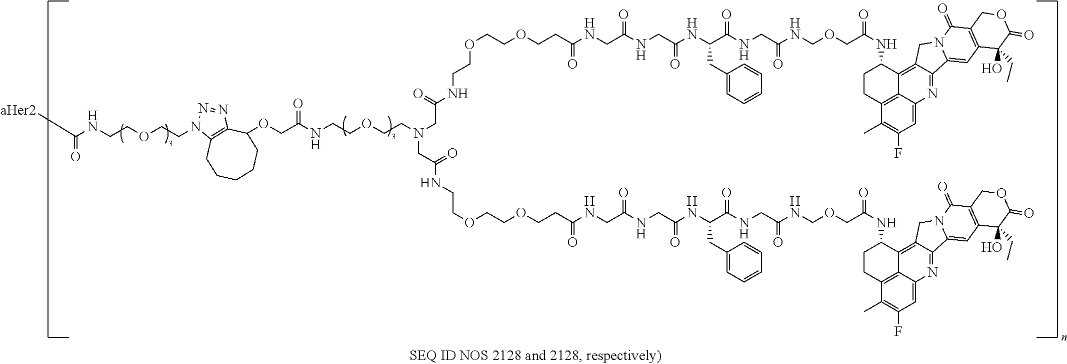 |
| 48 | FelD1- (AL11a- LP15)n | 7.0 | 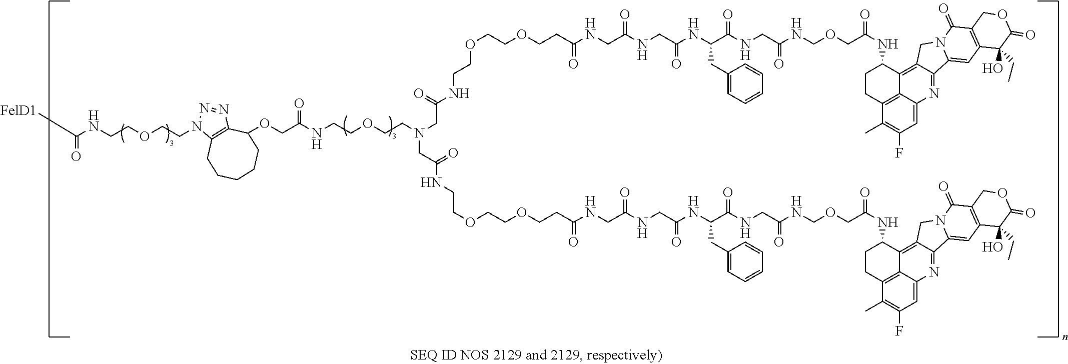 |
| 49 | aHer2- (BL1- LP1)n | 7.7 | 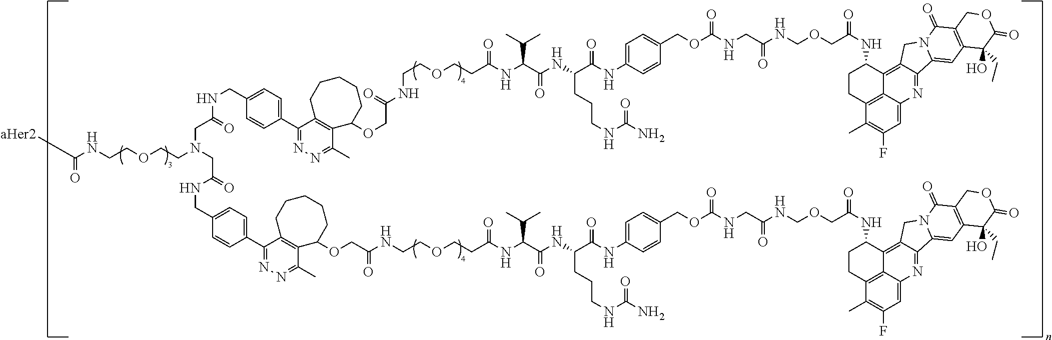 |
| 50 | FelD1- (BL1- LP1)n | 7.6 | 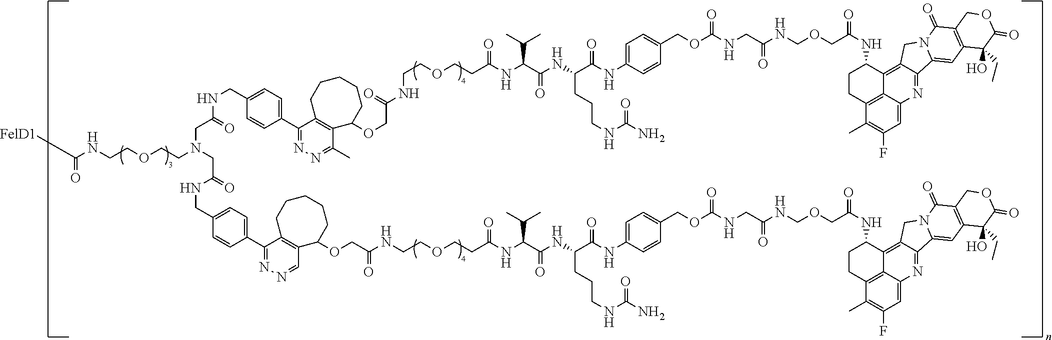 |
| 51 | aHer2- (BL1- LP13)n | 14.88 | 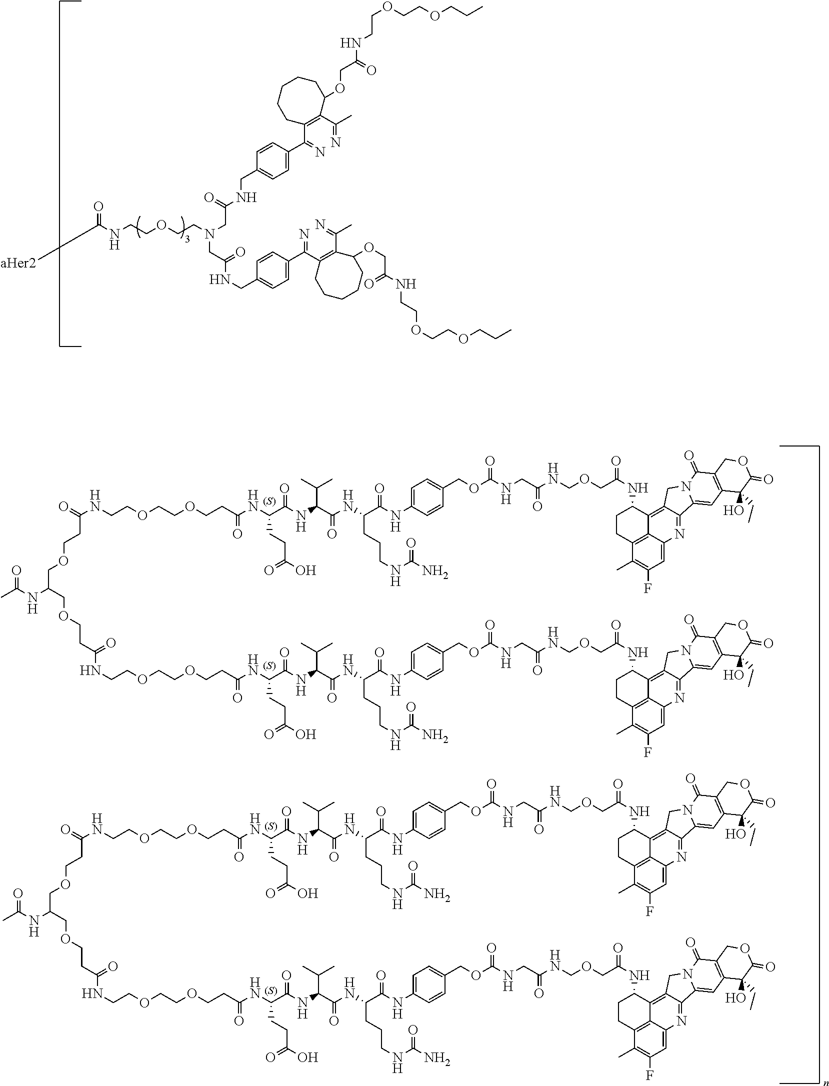 |
| 52 | FelD1- (BL1- LP13)n | 15.00 | 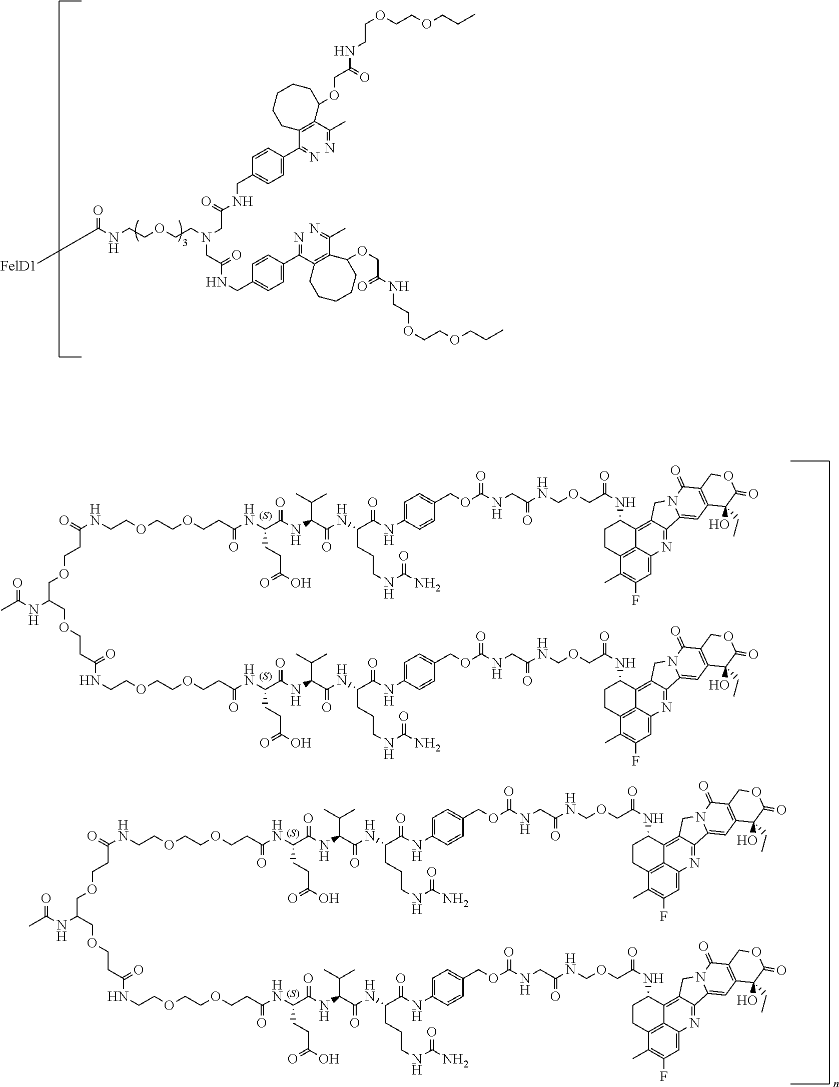 |
| 53 | aHer2- (BL3b- LP1)n | 7.5 | 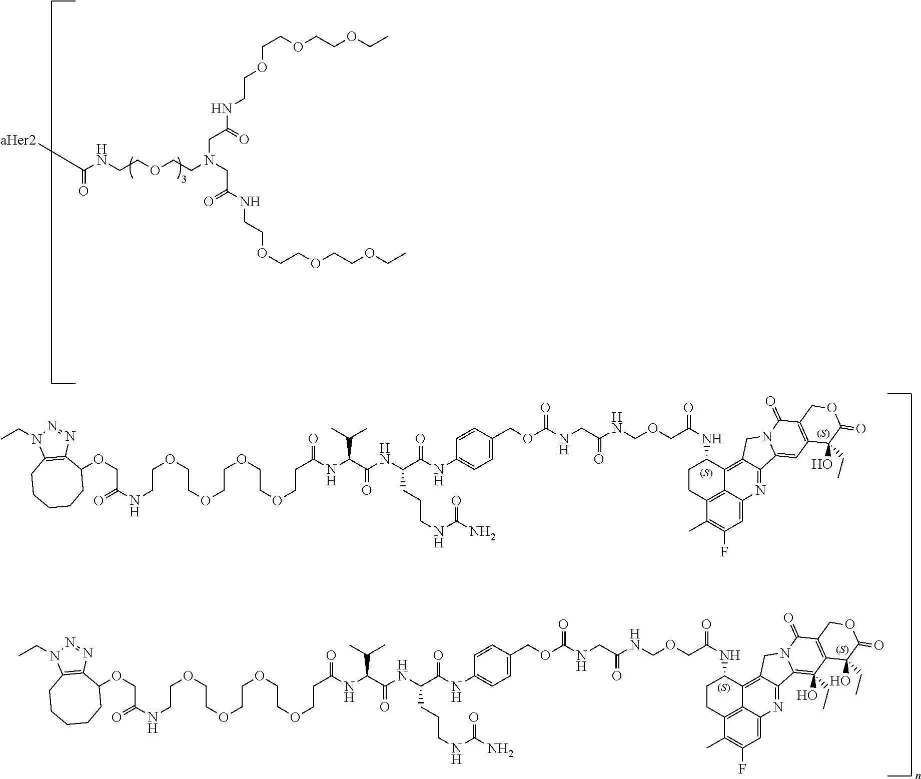 |
| 54 | FelD1- (BL3b- LP1)n | 7.1 | 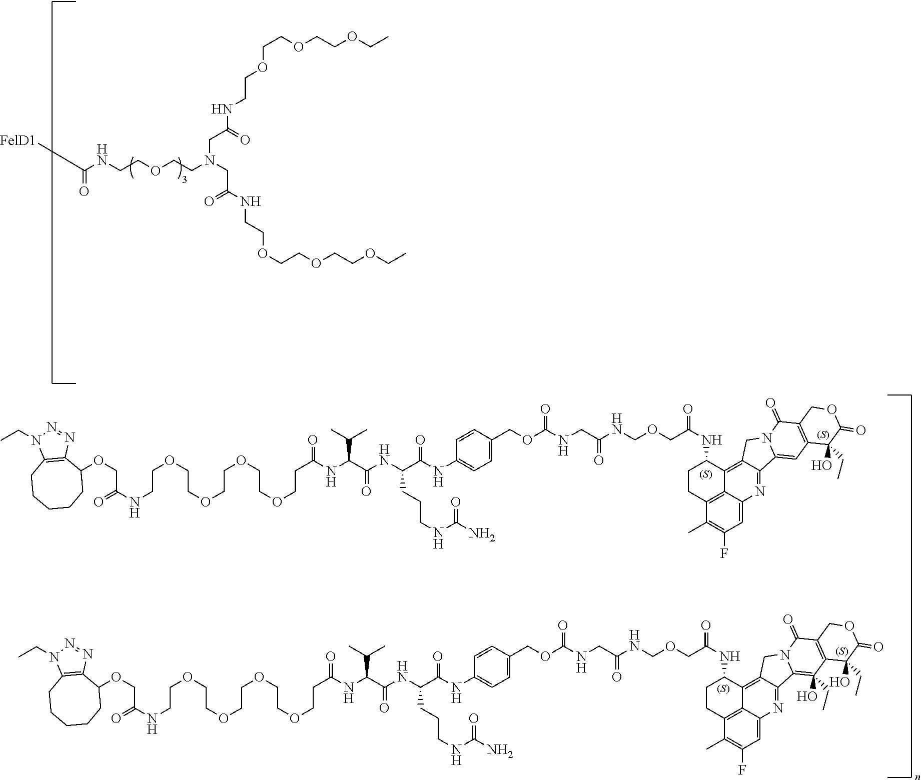 |
| 55 | aHer2- (Mc- GGFG- DXD)n | 7.7 | 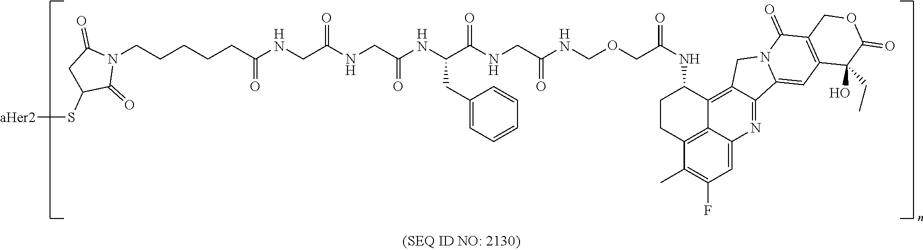 |
| 55-1 | aHer2- (Mc- GGFG- DXD)n | 7.7 | 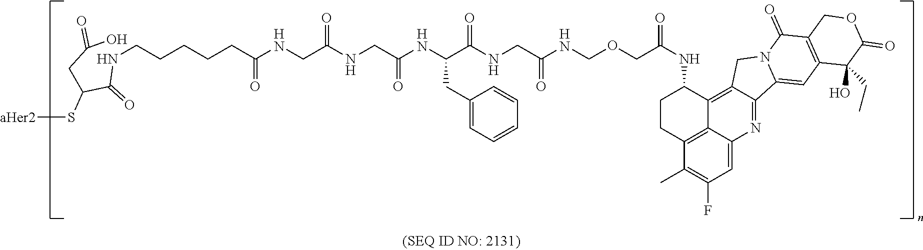 |
| 55-2 | aHer2- (Mc- GGFG- DXD)n | 7.7 | 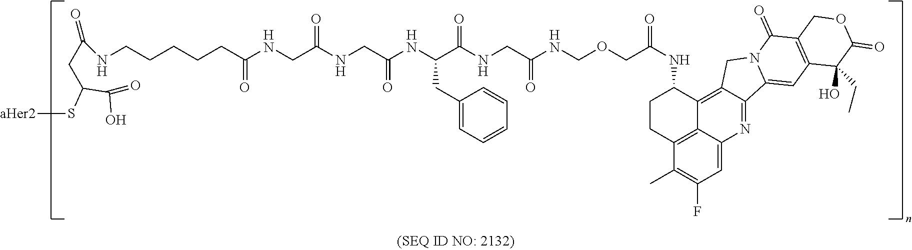 |
| 56 | FelD1- (Mc- GGFG- DXD)n | 7.5 | 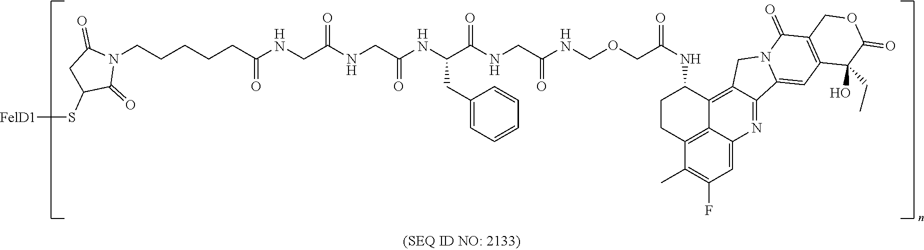 |
| 56-1 | FelD1- (Mc- GGFG- DXD)n | 7.5 | 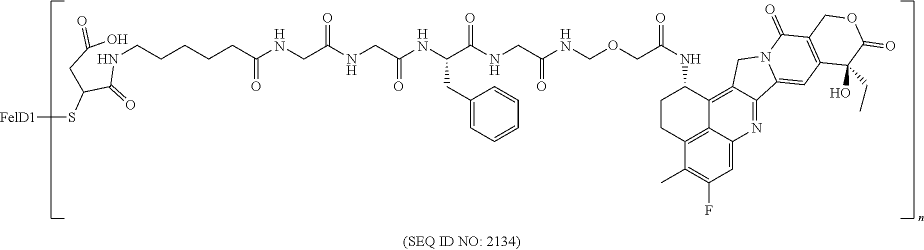 |
| 56-2 | FelD1- (Mc- GGFG- DXD)n | 7.5 | 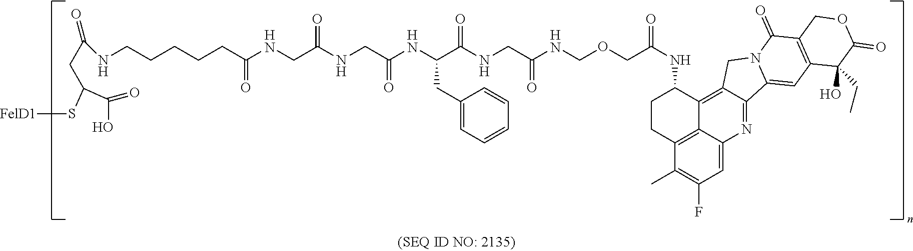 |
| 57 | aHer2- (Mc- GGFG- DXD)n | 7.1 | 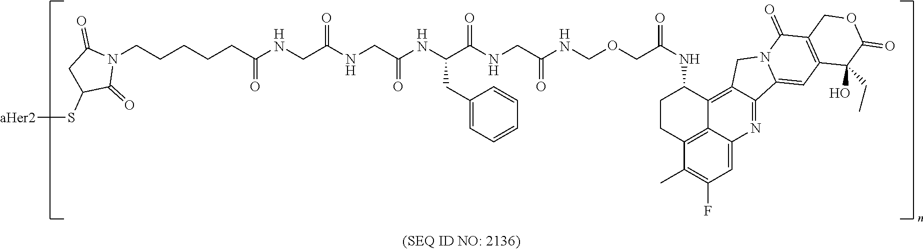 |
| 57-1 | aHer2- (Mc- GGFG- DXD)n | 7.1 | 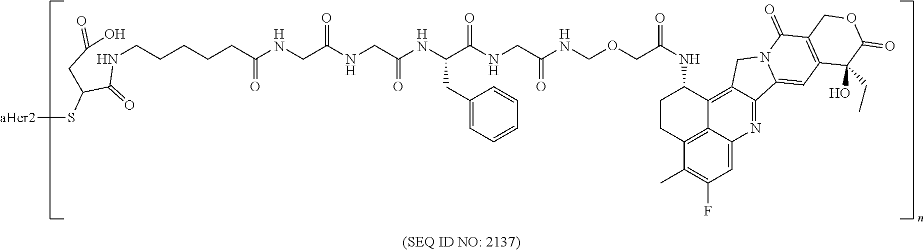 |
| 57-2 | aHer2- (Mc- GGFG- DXD)n | 7.1 | 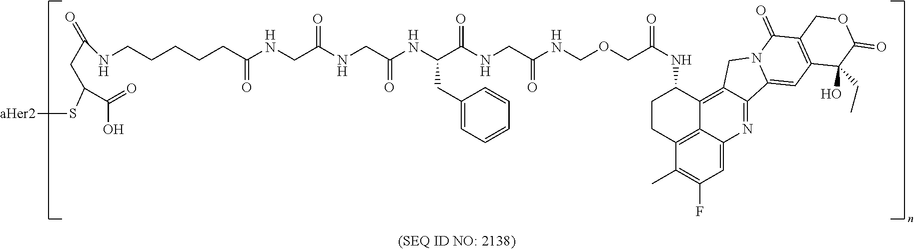 |
| 58 | FelD1- (Mc- GGFG- DXD)n | 7.0 | 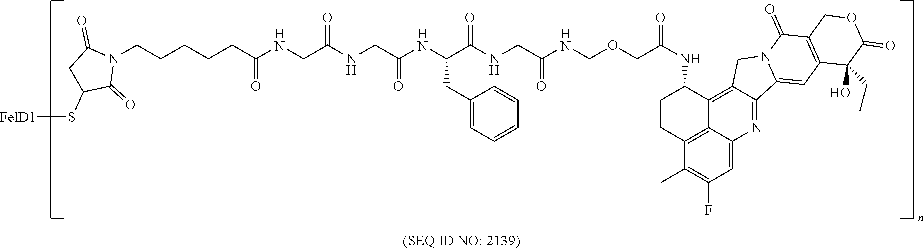 |
| 58-1 | FelD1- (Mc- GGFG- DXD)n | 7.0 | 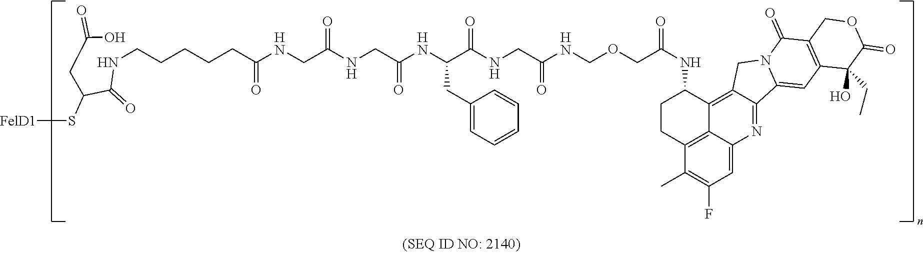 |
| 58-2 | FelD1- (Mc- GGFG- DXD)n | 7.0 | 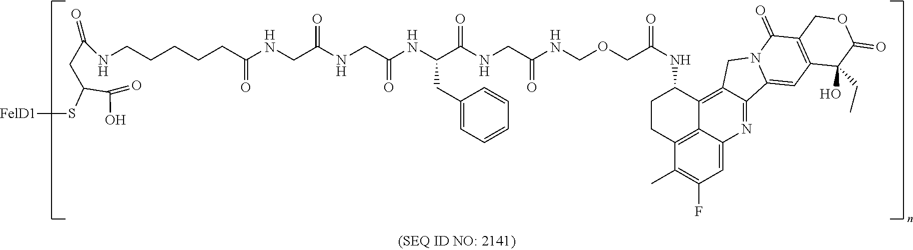 |

### List of ADO OAR and MS, as Well Associated Handle and Linker-Payload

| ADC | | | | Handle | | Linker-payload | |
|---|---|---|---|---|---|---|---|
| # | Code# | DAR | ES-MS | Code# | MS of handle | code# | MS of LP |
| 1 | aHer2 Ab | | 145143 | / | / | / | / |
| 2 | FeID1 Ab | | 145452 | / | / | / | / |
| 3 | aHer2-(AL11a)n | 4 | 145951 | AL11a | 218.14 | / | / |
| 4 | FeID1-(AL11a)n | 4 | 146254 | AL11a | 218.14 | / | / |
| 5 | aHer2-(AL2a)n | 4 | 146626 | AL2a | 344.23 | / | / |
| 6 | FeID1-(AL2a)n | 4 | 146935 | AL2a | 344.23 | / | / |
| 7 | aHer2-(AL3a)n | 3.94 | 146722 | AL3a | 368.23 | / | / |
| 8 | FeID1-(AL3a)n | 4 | 147031 | AL3a | 368.23 | / | / |
| 9 | aHER2-(AL7)n | 3.93 | 145868 | AL7 | 201.10 | / | / |
| 10 | FeID1-(AL7)n | 3.92 | 146179 | AL7 | 201.10 | / | / |
| 11 | aHER2-(AL6)n | 3.6 | 145818 | AL6 | 187.09 | / | / |
| 12 | aHer2-(BL1)n | 3.9 | 147770 | BL1 | 674.34 | / | / |
| 13 | FeID1-(BL1)n | 3.9 | 148082 | BL1 | 674.34 | / | / |
| 14 | aHer2-(BL3b)n | 3.8 | 147915 | BL3b | 708.80 | / | / |
| 15 | FeID1-(BL3b)n | 3.8 | 148231 | BL3b | 708.80 | / | / |
| 16 | aHer2-(AL6-LP1)n | 3.6 | Reduced ESI-MS | AL6 | 187.09 | LP1 | 1395.64 |
| 17 | aHer2-(AL6-LP15)n | 6.2 | Reduced ESI-MS | AL6 | 187.09 | LP15 | 2435.05 |
| 18 | aHer2-(AL6-LP13)n | 6.5 | Reduced ESI-MS | AL6 | 187.09 | LP13 | 3067.35 |
| 19 | aHer2-(AL7-LP1)n | 3.7 | 151343 | AL7 | 201.10 | LP1 | 1395.64 |
| 20 | FeID1-(AL7-LP1)n | 3.9 | 151666 | AL7 | 201.10 | LP1 | 1395.64 |
| 21 | aHer2-(AL7-LP13)n | 7.3 | 158032 | AL7 | 201.10 | LP13 | 3067.35 |
| 22 | FeID1-(AL7-LP13)n | 7.3 | 158351 | AL7 | 201.10 | LP13 | 3067.35 |
| 23 | aHer2-(AL7-LP2)n | 3.83 | 151473 | AL7 | 201.10 | LP2 | 1425.65 |
| 24 | FeID1-(AL7-LP2)n | 3.80 | 151784 | AL7 | 201.10 | LP2 | 1425.65 |
| 25 | aHer2-(AL7-LP3)n | 3.88 | 151636 | AL7 | 201.10 | LP3 | 1466.68 |
| 26 | FeID1-(AL7-LP3)n | 3.82 | 151949 | AL7 | 201.10 | LP3 | 1466.68 |
| 27 | aHer2-(AL7-LP4)n | 3.84 | 151639 | AL7 | 201.10 | LP4 | 1467.66 |
| 28 | FeID1-(AL7-LP4)n | 3.82 | 151952 | AL7 | 201.10 | LP4 | 1467.66 |
| 29 | aHer2-(AL7-LP5)n | 3.80 | 151642 | AL7 | 201.10 | LP5 | 1466.71 |
| 30 | FeID1-(AL7-LP5)n | 3.69 | 151954 | AL7 | 201.10 | LP5 | 1466.71 |
| 31 | aHer2-(AL7-LP7)n | 3.70 | 151713 | AL7 | 201.10 | LP7 | 1485.69 |
| 32 | FeID1-(AL7-LP7)n | 3.68 | 152027 | AL7 | 201.10 | LP7 | 1485.69 |
| 33 | aHer2-(AL7-LP9)n | 3.81 | 151401 | AL7 | 201.10 | LP9 | 1409.66 |
| 34 | FeID1-(AL7-LP9)n | 3.89 | 151712 | AL7 | 201.10 | LP9 | 1409.66 |
| 35 | aHer2-(AL7-LP12)n | 3.82 | 152150 | AL7 | 201.10 | LP12 | 1595.76 |
| 36 | FeID1-(AL7-LP12)n | 3.83 | 152462 | AL7 | 201.10 | LP12 | 1595.76 |
| 37 | aHer2-(AL7-LP5D)n | 3.89 | 152152 | AL7 | 201.10 | LP5D | 1595.76 |
| 38 | aHer2-(AL7-LP5D)n | 3.90 | 152465 | AL7 | 201.10 | LP5D | 1595.76 |
| 39 | aHer2-(AL4c-LP1B)n | 3.49 | 151990 | AL4c | 356.23 | LP1B | 1402.60 |
| 40 | FeID1-(AL4c-LP1B)n | 3.45 | 152300 | AL4c | 356.23 | LP1B | 1402.60 |
| 41 | aHer2-(AL4c-LP13C)n | 7.07 | 158211 | AL4c | 356.23 | LP13C | 2956.25 |
| 42 | aHer2-(AL4c-LP13C)n | 7.11 | 158521 | AL4c | 356.23 | LP13C | 2956.25 |
| 43 | aHer2-(AL11a-LP1)n | 3.68 | 146502 | AL11a | 218.14 | LP1 | 1395.64 |
| 44 | FeID1-(AL11a-LP1)n | 4 | 151841 | AL11a | 218.14 | LP1 | 1395.64 |
| 45 | aHer2-(AL11a-LP13)n | 7.8 | 158223 | AL11a | 218.14 | LP13 | 3067.35 |
| 46 | FeID1-(AL11a-LP13)n | 7.3 | 158529 | AL11a | 218.14 | LP13 | 3067.35 |
| 47 | a Her2-(AL11a-LP15)n | 7.6 | 155695 | AL11a | 218.14 | LP15 | 2435.05 |
| 48 | FeID1-(AL11a-LP15)n | 7.0 | 155998 | AL11a | 218.14 | LP15 | 2435.05 |
| 49 | aHer2-(BL1-LP1)n | 7.7 | 158723 | BL1 | 674.34 | LP1 | 1395.64 |
| 50 | FeID1-(BL1-LP1)n | 7.6 | 159038 | BL1 | 674.34 | LP1 | 1395.64 |
| 51 | aHer2-(BL1-LP13)n | 14.88 | 172107 | BL1 | 674.34 | LP13 | 3067.35 |
| 52 | FeID1-(BL1-LP13)n | 15.00 | 172419 | BL1 | 674.34 | LP13 | 3067.35 |
| 53 | aHer2-(BL3b-LP1)n | 7.5 | 159093 | BL3b | 708.80 | LP1 | 1395.64 |
| 54 | FeID1-(BL3b-LP1)n | 7.1 | 159398 | BL3b | 708.80 | LP1 | 1395.64 |
| 55 | aHer2-(Mc-GGFG-DXD)n | 7.7 | Reduced ESI-MS | / | | / | |
| | (SEQ ID NO: 2130) | | | | | | |
| 56 | FeID1-(Mc-GGFG-DXD)n | 7.5 | Reduced ESI-MS | / | | / | |
| | (SEQ ID NO: 2133) | | | | | | |
| 57 | aHer2-(Mc-GGFG-DXD)n | 7.1 | Reduced ESI-MS | / | | / | |
| | (SEQ ID NO: 2136) | | | | | | |
| 58 | FeID1-(Mc-GGFG-DXD)n | 7.0 | Reduced ESI-MS | / | | / | |
| | (SEQ ID NO: 2139) | | | | | | |
| 59 | DS8201a | 7.8 | | | | | |

### Characterization of ADCs

### SDS-PAGE for Analysis of ADC Integrity and Purity

In one method, SDS-PAGE running conditions include non-reduced and reduced samples (1-2pg) along with Precision Plus Protein Dual Color Standards (Bio-rad, 500 μl, Cat #1610374) are loaded per lane in (1.0 mm×10 well) Novex 4-20% No Tris-Glycine Gel and is run at 180V, 300 mA, for 80 minutes. A non-reduced sample is prepared using NuPAGE® LDS Sample Buffer (4×) (Thermo Fisher Scientific, Cat #1887691) and the reduced sample is prepared with SDS sample buffer (4×) containing 10% sample reducing agent (10×) (Thermo Fisher Scientific, Cat #1769410).

Molecular weights of the antibodies and ADCs on SDS-PAGE are determined under non-reducing and reducing conditions. The mass shifts may not be obvious under non-reducing conditions due to relatively small percentages of mass changes. However, the masses of the heavy chains are increased from the naked antibodies to the azido-functionalized antibodies, and further to the ADC conjugates.

### Size Exclusion Chrmoatography (SEC) for ADC Analysis and Purification

To determine the purity of antibody drug conjugates, size exclusion chromatography is performed. Analytical SEC experiments are run using a Thermo UltiMate™ 3000 instrument, on a XBridge Protein BEH SEC Column (Waters, 200 Å, 3.5 μm, 7.8 mm×300 mm), and each sample (30-40 μg, 20 μL) are run at flow rate of 0.5 mL/min using PBS pH 7.4 with 15% 2-propanol and monitored at λ280 nm using Thermo DAD-3000 RS Rapid Separation Diode Array Detector.

ADCs are purified by Size Exclusion Chromatography (SEC) and concentrated by using ultra centrifugation. To separate the antibody drug conjugates from the reaction mixture, preparative SEC purifications are performed using the ÄKTA instrument from GE Healthcare, on a Superdex® 200 increase 10/300 GL (1.0×30 cm) column, at the flow rate of 0.6 mL/min eluting with BupH at pH 7.4, and monitored at λ280 nm. To concentrate the product Amicon @ Ultra-4 Centrifugal Filters (Ultracel-10K) are used in Allegra x-12r centrifuge and the solution is stirred after each concentration to avoid high aggregation.

### LC-ESI-MS for Intact Mass Analysis of Antibody and ADC

Measurement of intact mass for the ADC samples by LC-ESI-MS was performed to determine payload distribution profile and to calculate the average DAR. Each testing sample (0.5-1 μg) was loaded onto Waters Protein BEH C4 Column (300 Å, 1.7 μm, 2.1 mm×50 mm; Cat No. 186004495) with different gradients of the Mobile Phase A (ddH2O with 0.1% FA) and Mobile Phase B (ACN with 0.1% FA) (as shown in the table below), at the flow rate of 0.25 μL/min, and monitored at λ280 nm. Then the product was eluted, and the mass spectra was acquired by Thermo Q EXACTIVE HF-X.

| No. | Time (min) | Flow (μL/min) | % B | Curve |
|---|---|---|---|---|
| 1 | 0.000 | Equilibration | | |
| 2 | 0.000 | 0.250 | 10.0 | 5 |
| 3 | New Row | | | |
| 4 | 0.000 | Run | | |
| 5 | 0.000 | 0.250 | 10.0 | 5 |
| 6 | 3.000 | 0.250 | 10.0 | 5 |
| 7 | 7.000 | 0.250 | 90.0 | 5 |
| 8 | 8.000 | 0.250 | 90.0 | 5 |
| 9 | 8.100 | 0.250 | 10.0 | 5 |
| 10 | 10.000 | 0.250 | 10.0 | 5 |
| 11 | New Row | | | |

| | | |
|---|---|---|
| 12 | 10.000 | Stop Run |

### Example 28. In Vitro Cell Based Assays and Results

The EC50 values for the ADCs and control ADCs, as well the free payload (DXD and Gly-NH2-CH2DXD) are summarized in Table 11, below. The anti-Her2 viability assay protocol is following.

### Materials

96 well, BioCoat cellware, poly-D-lysine, white, opaque bottom [Thermo #136101]. 96 well deep well plates, 1.1 mL round wells [Axygen Scientific, cat #P-DW-11-C-S, VWR #47734-788]—large volume dilution plate. Corning 75 cm2 flask [Corning cat #430641U]. Reagent reservoirs, 50 mL, white, individually wrapped [VWR, cat #89094-682]. Reagent reservoirs, 25 mL, white, polystyrene, 5/bag [VWR, cat #89094-662]. Centrifuge conical tube, PP, 50 mL [Corning, cat #352098]. Centrifuge conical tube, PP, 15 mL [Corning, cat #352097]. Envision plate reader [Perkin Elmer Model #2104]. Top Seal A Plus [Perkin Elmer, cat #6050185]. McCoy's Medium 5A [Irvine Scientific, cat #9090]. DME High Glucose [Irvine Scientific, cat #9033]. MEM Earle's Salts, [Irvine Scientific, cat #9126]. RPMI medium 1640 [Irvine Scientific, cat #9160]. Penicillin-Streptomycin L-glutamine Solution 100× [ThermoFisher Scientific, cat #10378016]. PBS 1× without calcium and magnesium salts [Irvine Scientific, cat #9240]. Trypsin-EDTA, 0.025% Trypsin & 0.75 mM EDTA (1×), w/o Ca2+& Mg2+[Millipore, cat #SM-2004-C]. Fetal Bovine Serum [Saradigm, cat #1500-500]. DMSO Dimethyl sulfoxide, cell culture tested [ATCC, cat #4-X]. CellTiter-Glo 2.0 [Promega, cat #G9243]. Bovine serum albumin solution, protease free [Sigma, cat #A8577]. Opti-MEM reduced serum media [Gibco, cat #31985-070].

### Preparation of the Assay Media

0.1% BSA was added to the Opti-MEM reduced serum media [Gibco, cat #31985-070]. This assay media was used for the dilution of ADC and free drug.

### Procedures

Cells were seeded in their growth media into 96 well plates (Thermo #136101) one day prior to adding ADCs: 1000/well SKBR3 cells in 80 uL media (target positive cells), 1000/well NCI-N87 cells in 80 uL media (target positive cells), 1000/well Calu-3 in 80 uL media (target positive cells), and 800/well NCI-H1975 cells in 80ul media (target negative cells).

Serial dilutions of free drug in 100% DMSO (1:4 10 pts starting from 100 uM) were prepared. Serially diluted payload was transferred into Opti-Mem with 0.1% BSA (10 uL into 990 uL media). Serial dilutions of ADCs/isotype controls at 1:4 in Opti-Mem with 0.1% BSA (starting from 5×1000 nM) were prepared. 20ul assay media diluted ADC/isotype conjugates as well as free drug were transferred to above cells. The plates were incubated at 37° C., 5% CO₂ for 6 days. Plates were developed by adding 100ul CellTiter Glo 2.0. RT 5-10 min. Plates were read with Envision plate reader. Data was analyzed using Prism. Only inner 60 well are used for the assay. The edge wells were filled with media to prevent dry out.

### SKBR3 Cell-Based Assay

The cell line used in the anti-proliferation assays was SK-BR-3, a human breast, adenocarcinoma (pleural effusion) cell line; The cells were grown in McCoy's 5a Medium+10% FBS. To run the assay, the cells (80 μl, 1000 cells) were added to each well in a 96-well plate and incubated for 24 hours at 37° C. with CO₂. Next, the cells were treated with test compounds (20 μl) at various concentrations in appropriate cell culture medium (total volume, 0.1 mL). The control wells contain cells and the medium but lack the test compounds. The plates were incubated for 144 hours at 37° C. with CO₂. CTG reagent was then added to the wells (100 μl). After the plates were shaken for 10 min and then incubated for 10 min at room temperature, paste the clear bottom with white back seal and record luminescence with Envision. The inhibition % was calculated according to the following equation: inhibition %=[1-(assay-blank)/(control-blank)]×100.

TABLE 11
List of ADCs and Payloads in vitro Cell Killing Activity

| | | | SKBR3 | | N87 | | Calu-3 | | JIMT-1 | | H1975 | |
|---|---|---|---|---|---|---|---|---|---|---|---|---|
| | | | IC50 | % | | % | IC50 | % | IC50 | % | IC50 | % |
| # | ADC# | DAR | (nM) | Kill | IC50 (nM) | Kill | (nM) | Kill | (nM) | Kill | (nM) | Kill |
| | DXd | | 1.02 | 97.0 | 2.59 | 98.6 | 1.30 | 94.9 | 4.41 | 94.5 | 3.45 | 99.3 |
| 59 | DS8201a | 7.7 | 0.197 | 92.9 | 1.07 | 98.6 | 0.301 | 94.5 | 306 | 83.6 | 263 | 80.4 |
| | Cont. | 7.5 | 95.3 | 88.0 | 168.00 | 83.4 | 157 | 87.9 | >400 | 44.7 | 689 | 51.6 |
| | DS8201a | | | | | | | | | | | |
| | P1 | / | 5.81 | 96.0 | 9.25 | 98.0 | 6.78 | 93.1 | 26.6 | 89.8 | 23.9 | 96.4 |
| 19 | aHer2- | 3.7 | 0.114 | 91.9 | 1.17 | 94.4 | 0.256 | 92.9 | 248 | 51.7 | 322 | 57.0 |
| | (AL7- | | | | | | | | | | | |
| | LP1)₄ | | | | | | | | | | | |
| 20 | FeID1- | 3.9 | 156 | 84.1 | 273.30 | 59.7 | >400 | 83.1 | >400 | 31.6 | >400 | 33.6 |
| | (AL7- | | | | | | | | | | | |
| | LP1)₄ | | | | | | | | | | | |
| 49 | aHer2- | 7.7 | 0.160 | 92.1 | 0.682 | 97.9 | 0.282 | 92.8 | >400 | 47.4 | 366 | 50.6 |
| | (BL1- | | | | | | | | | | | |
| | (LP1)₂)₄ | | | | | | | | | | | |
| 50 | FeID1- | 7.6 | 89.1 | 88.5 | 210 | 75.3 | 142 | 89.8 | >400 | 49.0 | 331 | 56.2 |
| | (BL1- | | | | | | | | | | | |
| | (LP1)₂)₄ | | | | | | | | | | | |
| 43 | aHer2- | 3.68 | 0.152 | 90.1 | 0.594 | 89.4 | 0.293 | 91.6 | >400 | 16.4 | >400 | 12.6 |
| | (AL11a- | | | | | | | | | | | |
| | LP1)₄ | | | | | | | | | | | |
| 44 | FeID1- | 4.00 | >400 | 26.6 | >400 | 15.3 | >400 | 31.7 | >400 | 14.9 | >400 | 4.0 |
| | (AL11a- | | | | | | | | | | | |
| | LP1)₄ | | | | | | | | | | | |
| 21 | aHer2- | 7.3 | 0.123 | 90.8 | 0.648 | 94.9 | 0.216 | 91.2 | >400 | 9.3 | >400 | 7.9 |
| | (AL7- | | | | | | | | | | | |
| | LP13)n | | | | | | | | | | | |
| 22 | FeID1- | 7.3 | >400 | 12.5 | >400 | 11.1 | >400 | 25.6 | >400 | 7.8 | >400 | 1.7 |
| | (AL7- | | | | | | | | | | | |
| | LP13)n | | | | | | | | | | | |
| 45 | aHer2- | 7.8 | 0.151 | 91.7 | 0.607 | 95.6 | 0.262 | 91.5 | >400 | 14.7 | >400 | 8.3 |
| | (AL11a- | | | | | | | | | | | |
| | LP13)n | | | | | | | | | | | |
| 46 | FeID1- | 7.3 | >400 | 9.4 | >400 | 4.8 | >400 | 15.9 | >400 | 10.4 | >400 | 4.1 |
| | (AL11a- | | | | | | | | | | | |
| | LP13)n | | | | | | | | | | | |
| 39 | aHer2- | 3.49 | 0.256 | 86.6 | 10.4 | 72.0 | 1.83 | 80.4 | >400 | 12.3 | >400 | 11.5 |
| | (AL4c- | | | | | | | | | | | |
| | LP1B)n | | | | | | | | | | | |
| 40 | FeID1- | 3.45 | 11.4 | 91.5 | 71.5 | 96.0 | 25.1 | 92.5 | 252 | 63.5 | 263 | 68.0 |
| | (AL4c- | | | | | | | | | | | |
| | LP1B)n | | | | | | | | | | | |
| 41 | aHer2- | 7.07 | 0.106 | 92.1 | 0.638 | 91.2 | 0.241 | 90.0 | >400 | 9.6 | >400 | 9.7 |
| | (AL4c- | | | | | | | | | | | |
| | LP13C)n | | | | | | | | | | | |
| 42 | aHer2- | 7.11 | 13.3 | 90.5 | 49.3 | 85.6 | 24.6 | 88.6 | >400 | 18.3 | >400 | 7.9 |
| | (AL4c- | | | | | | | | | | | |
| | LP13C)n | | | | | | | | | | | |
| | P2 | / | 5.89 | 97.0 | 8.93 | 98.4 | 5.68 | 93.2 | 23.8 | 92.7 | 19.9 | 98.0 |
| 23 | aHer2- | 3.83 | 0.164 | 92.8 | 1.71 | 97.8 | 0.265 | 93.8 | 192 | 68.5 | 223 | 72.1 |
| | (AL7- | | | | | | | | | | | |
| | LP2)₄ | | | | | | | | | | | |
| 24 | FeID1- | 3.80 | 47.8 | 91.0 | 147.40 | 89.5 | 70.4 | 90.8 | 183 | 64.5 | 233 | 71.4 |
| | (AL7- | | | | | | | | | | | |
| | LP2)₄ | | | | | | | | | | | |
| | P3 | / | 11.6 | 94.3 | 30.4 | 95.5 | 15.9 | 90.7 | 88.6 | 81.3 | 54.5 | 88.5 |
| 25 | aHer2- | 3.88 | 0.172 | 88.4 | 4.73 | 80.6 | 0.31 | 88.4 | >400 | 9.7 | >400 | 5.3 |
| | (AL7- | | | | | | | | | | | |
| | LP3)₄ | | | | | | | | | | | |
| 26 | FeID1- | 3.82 | 121 | 85.7 | >400 | 45.6 | 341 | 73.9 | >400 | 8.9 | >400 | 7.2 |
| | (AL7- | | | | | | | | | | | |
| | LP3)₄ | | | | | | | | | | | |
| | P4 | / | 6.63 | 96.6 | 9.84 | 97.7 | 5.67 | 93.7 | 20.7 | 92.0 | 24.1 | 96.8 |
| 27 | aHer2- | 3.84 | 0.186 | 90.3 | 2.07 | 87.5 | 0.222 | 91.9 | >400 | 22.0 | >400 | 10.9 |
| | (AL7- | | | | | | | | | | | |
| | LP4)₄ | | | | | | | | | | | |
| 28 | FeID1- | 3.82 | 212 | 57.7 | >400 | 24.8 | >400 | 38.9 | >400 | 15.5 | >400 | 8.4 |
| | (AL7- | | | | | | | | | | | |
| | LP4)₄ | | | | | | | | | | | |
| | P5 | / | 4.51 | | | | | | | | | |
| 29 | aHer2- | 3.80 | 0.216 | 90.6 | 2.19 | 91.7 | 0.327 | 90.7 | >400 | 34.1 | >400 | 24.7 |
| | (AL7- | | | | | | | | | | | |
| | LP5)₄ | | | | | | | | | | | |
| 30 | FeID1- | 3.69 | 204 | 79.9 | >400 | 41.9 | 178 | 75.3 | >400 | 30.1 | >400 | 19.5 |
| | (AL7- | | | | | | | | | | | |
| | LP5)₄ | | | | | | | | | | | |
| 37 | aHer2- | 3.89 | 0.139 | 93.0 | 1.46 | 98.2 | 0.190 | 94.0 | 218 | 84.8 | 209 | 83.1 |
| | (AL7- | | | | | | | | | | | |
| | LP5D)₄ | | | | | | | | | | | |
| 38 | FeID1- | 3.90 | 88.2 | 87.7 | 169 | 82.3 | 95.3 | 89.0 | 315 | 57.0 | 280 | 66.7 |
| | (AL7- | | | | | | | | | | | |
| | LP5D)₄ | | | | | | | | | | | |
| 35 | aHer2- | 3.82 | 0.210 | 90.7 | 3.14 | 95.0 | 0.302 | 92.2 | >400 | 46.3 | 355 | 52.1 |
| | (AL7- | | | | | | | | | | | |
| | LP12)₄ | | | | | | | | | | | |
| 36 | FeID1- | 3.83 | 184 | 81.7 | >400 | 44.2 | 121 | 73.8 | >400 | 27.3 | >400 | 21.7 |
| | (AL7- | | | | | | | | | | | |
| | LP12)₄ | | | | | | | | | | | |
| | P7 | / | 1.98 | 97.0 | 4.41 | 98.6 | 3.32 | 93.7 | 13.2 | 93.4 | 8.96 | 98.8 |
| 31 | aHer2- | 3.70 | 0.303 | 95.1 | 2.04 | 99.0 | 0.581 | 94.1 | 158 | 90.1 | 81.6 | 90.8 |
| | (AL7- | | | | | | | | | | | |
| | LP7)₄ | | | | | | | | | | | |
| 32 | FeID1- | 3.68 | 21.1 | 94.0 | 51.69 | 96.4 | 31.5 | 92.8 | 127 | 86.0 | 109 | 86.0 |
| | (AL7- | | | | | | | | | | | |
| | LP7)₄ | | | | | | | | | | | |
| | P9 | / | 69.50 | | | | | | | | | |
| 33 | aHer2- | 3.81 | 0.14 | 91.6 | 0.82 | 92.1 | 0.661 | 83.7 | >400 | 10.3 | >400 | 14.9 |
| | (AL7- | | | | | | | | | | | |
| | LP9)₄ | | | | | | | | | | | |
| 34 | FeID1- | 3.89 | 33.1 | 88.4 | 85.56 | 81.7 | 47.7 | 90.6 | >400 | 20.6 | >400 | 20.3 |
| | (AL7- | | | | | | | | | | | |
| | LP9)₄ | | | | | | | | | | | |

Metabolic studies of a drug candidate shed lights on the pharmacological and pharmacokinetic pathways. It may also provide essential information on drug safety and its potential toxicity, in addition to its in vivo and clinical efficacy.

In vitro stability of Linker-Prodrug in mouse whole blood Scheme 15. Metabolism of LP1 (M2980) in whole blood.

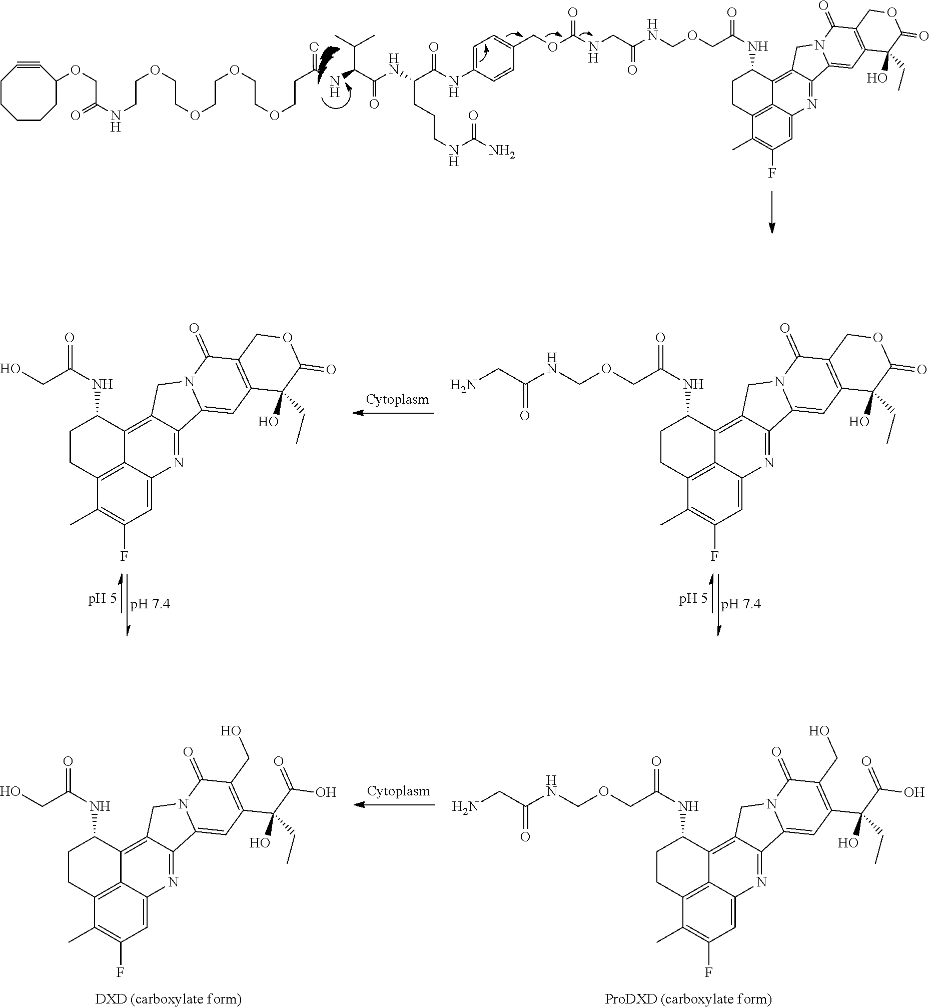

### Assay Protocol of Whole Blood Stability Test

Stock solution of test compound was provided in 100% DMSO. The stock solution for each compound was diluted into 500 μM with mixture of 50% acetonitrile, then diluted into mouse blood to achieve a final concentration of 1.0 μM. 1.0 μM of test compound in duplicate was incubated in blood at 37° C. Aliquots of 50 uL sample was collected at 0, 15, 30, 60, 120, 240, 480 min, and 24 hours. Reactions were terminated at various time points (0, 15, 30, 60, 120, 240, 480 min, 24 hours) by adding 200 μL of ice-cold acetonitrile containing internal standard with 1% formic acid and then ultrasound for 30 seconds. Plate was centrifuged (4000 rpm, 15 min). 50 μL of supernatants were transferred into a daughter plate containing 200 μL of H₂O in each well. The samples were mixed well and analyzed with UPLC-MS/MS.

### LC-MS/MS Analysis

A Waters liquid chromatographic system was used. Detection was performed on AP14000 Q-Trap and AP15500 mass spectrometer equipped with TurbolonSpray (ESI) Interface (Applied Biosystems, Concord, Ontario, Canada). Analyst 1.5 and 1.6.2 software packages (Applied Biosystems) were used to control the LC-MS/MS system, as well as for data acquisition and processing.

Chromatographic separation was achieved on the Wates BEH C18 column (50*2.1 mm ID, 1.7 μm). The column temperature was maintained at ambient temperature (25° C.). The flow rate was maintained at 0.6 mL/min and the following mobile phases were used: water (0.1% formic acid, v/v) (phase A) and acetonitrile (0.1% formic acid, v/v) (phase B). LC-MS conditions are shown in Table 12 below.

TABLE 12
LC-MS Conditions

| Time | A % | B % |
|---|---|---|
| Initial | 95 | 5 |
| 1.20 | 20 | 80 |
| 1.40 | 20 | 80 |
| 1.41 | 95 | 5 |
| 1.60 | 95 | 5 |

Test compounds (1.0 μM) were incubated in blood at 37° C. in duplicate. Aliquots of 50 uL sample was collected at 0, 15, 30, 60, 120, 240, 480 min, and 24 hours. Reactions were terminated at various time points (0, 15, 30, 60, 120, 240, 480 min, 24 hours by adding 200 μL of ice-cold acetonitrile containing internal standard with 1% formic acid, then ultrasound for 30 seconds. The plate was centrifuged (4000 rpm, 15 min). 50 μL of supernatants were transferred into a daughter plate containing 200 μL of water in each well. Samples were mixed well and analyzed with UPLC-MS/MS.

TABLE 13
Test Results of LP1 (M2980) Stability in Whole Bood

| | | | | | | ProDXd |
|---|---|---|---|---|---|---|
| | | | | ProDXd | DXd | released + |
| | | | | released | released | DXd |
| Time | | | | from | from | released + |
| (hr) | DXd | ProDXd | M2980 | M2980 | M2980 | M2980 |
| 0 | 100.0% | 100.0% | 100.0% | 1.8% | 3.0% | 104.8% |
| 0.25 | 95.9% | 63.9% | 88.8% | 4.9% | 3.7% | 97.4% |
| 0.5 | 103.6% | 39.0% | 82.6% | 8.6% | 4.0% | 95.2% |
| 1 | 111.9% | 30.7% | 72.3% | 22.1% | 8.6% | 103.0% |
| 2 | 97.6% | 14.8% | 52.8% | 32.3% | 22.7% | 107.8% |
| 4 | 94.8% | 2.7% | 30.5% | 33.1% | 43.2% | 106.8% |
| 8 | 94.2% | 0.3% | 16.5% | 31.6% | 74.9% | 123.0% |
| 24 | 94.2% | 0.0% | 0.0% | 4.0% | 80.1% | 84.0% |

The % was calculated based on initial 1 uM as 100% and the standard concentration curve.

The result indicated M2980 (LP1) is metabolized to ProDXD, and then to DXd, which provides its unique pharmacokinetic and pharmacodynamic properties, which contributes to, at least partially, and result in its superior in vivo efficacy.

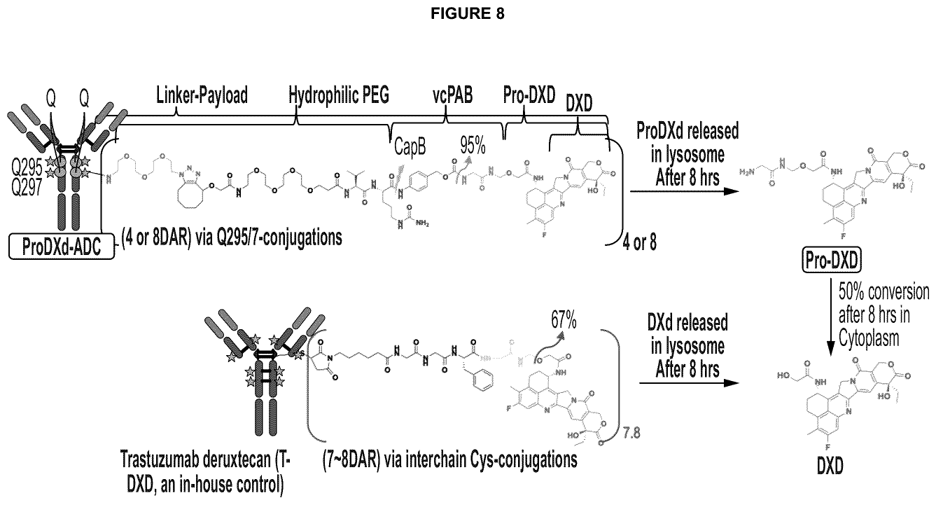

The therapeutic molecules as disclosed herein could be considered as a double prodrug approach. ADC is a prodrug of its payload, most commonly a cytotoxic agent, and a prodrug of DXd (ProDXd) was designed for conjugation to an antibody as an ADC payload. As it was schematically shown in FIG. 8, a specifically built-in cathepsin B cleavable linker in our ADC molecule was cleaved by cathepsin B to release ProDXd in lysosome. ProDXd is slowly metabolized to DXd in targeting cell—Cytoplasm; both DXd and ProDXd demonstrate Bystander effect in TME (tumor micro-environment). ProDXd has Lower pGP and BCRP efflux rates than DXd that reduced Cmax of DXD in circulation that reduced in vivo toxicity related to the payload. This double prodrug-payload releasing mechanism resulted in further improvement of the tolerability and safety and increase therapeutic index or therapeutic window.

To mimicry the LP1 (M2980) in a conjugated molecule, model compound M3385 was prepared using the straightforward Click chemistry as shown the scheme below:

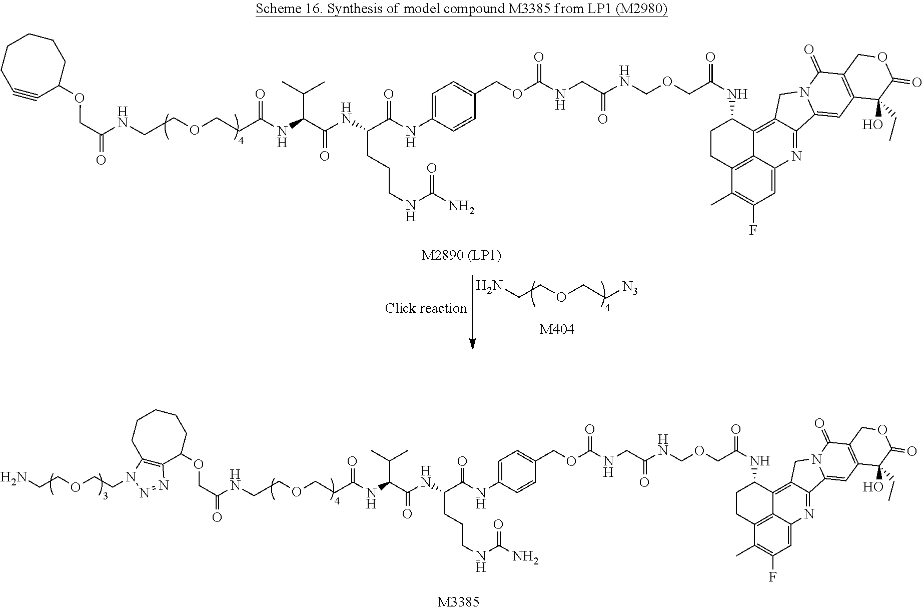

### Model M3385 Metabolism Studies Using Hepatocytes, Liver Microsomes or Liver S9, the Soluble Fractions of Homogenate of Hepatocytes.

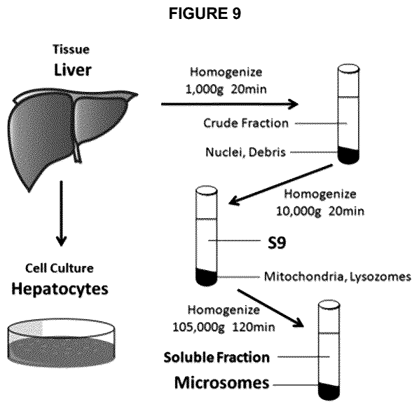

FIG. 9 shows the schematic process for the preparation of the liver S9 and the liver microsomes from hepatocytes. Briefly, hepatocytes are complete liver cells, containing various first-phase and second-phase enzymes that can mediate various metabolic reactions; therefore, it is a better in vitro model to test metabolites. Liver S9 is the supernatant obtained by grinding and centrifuging the liver cells. The enzymes content is lower compared to Hepatocytes. It mainly contains CYP enzymes and some biphasic enzymes (but no biphasic coenzyme), so additional coenzyme (NADPH and UDPGA, etc.) is needed to mediate the biphasic reaction.

Liver microsomes are the lower part obtained by grinding and centrifuging the liver cells and mainly mediate a phase reaction. Because the cell membrane is destroyed, the test-compound has no limitation to pass through the cell membrane and is directly exposed to the liver enzymes that are also in liver microsome and S9 (Fonsi et al., “High-Throughput Microsomal Stability Assay for Screening New Chemical Entities in Drug Discovery,” Journal of Biomolecular Screening 13(9):862-869 (2008), which is incorporated by reference herein in its entirety).

The metabolism of model compound M3385 was evaluated in human liver S9 with NADPH and UDPGA as follows:

Assay buffer: 100 mM potassium phosphate buffer (K+/Mg2+ buffer, pH7.4):

| | K₂HPO₄•3H₂O | KH₂PO₄ | MgCl₂•6H₂O | |
|---|---|---|---|---|
| | (g) | (g) | (g) | dH₂O |
| Reagent | FW: 228.22 | FW: 136.09 | FW: 203.3 | (mL) |
| 100 mM | 9.243 | 1.298 | 0.511 | 500 |

### Preparation of Cofactor Solution in the K+/Mg2+ Buffer:

| | NADPH | UDPGA | K⁺/Mg²⁺ | Total |
|---|---|---|---|---|
| | (MW: 833.4) | (MW: 646.2) | Buffer | volume |
| Cofactor solution | (mg) | (mg) | (mL) | (mL) |
| 8 mM NADPH | 14.24 | — | 2.136 | 2.136 |
| 20 mM UDPGA | — | 26 | 2.012 | 2.012 |

The initial stock of liver S9 was diluted in K+/Mg²⁺ buffer, pH 7.4 from 20 mg/ml to 2× Liver S9 (2 mg/mL) with alamethicin (50 μg/mL):

| Buffer | liver microsome | Alamethicin | Total |
|---|---|---|---|
| (μL) | (μL) | (10 mg/mL, μL) | (μL) |
| 4117 | 460 | 23 | 4600 |

### Assay Procedure:

2×LS9/compound M3385 solution was prepared.

Time zero/T0: 199 μL 2 mg/mL liver S9 solution; 100 μL of 8 mM NADPH solution; 100 μL of 20 mM UDPGA solution; and 1200 μL of ACN were added, vortexed at 1000 rpm for 5 min, then 1 μL of 4 mM test compound solution was added.

T240 with a co-factor: 199 μL 2 mg/mL liver S9 solution; 100 μL of 8 mM NADPH solution; and 100 μL of 20 mM UDPGA solution were added. The T240 sample was prewarmed at 37° C. for 5 min and 1 μL of 4 mM test compound solution was added. After 240 min incubation, 1200 μL of ACN was added and then vortexed at 1000 rpm for 5 min.

T240-without a co-factor: 199 μL 2 mg/mL liver S9 solution and 200 μL of buffer were added. The T240-w/o sample was prewarmed at 37° C. for 5 min and 1 μL of 4 mM test compound solution was added. After 240 min incubation, 1200 μL of ACN was added and then vortexed at 1000 rpm for 5 min.

Protein precipitation: quenched samples were centrifuged at 14000 rpm for 5 min.

Sample preparation: an aliquot of 1200 μL of the supernatant was evaporated under N2 stream until dry. Dried extracts were reconstituted with 200 μL of 25% aqueous ACN, vortexed for 2 minutes, centrifuged at 14000 rpm for 5 min, and then 3 μL of reconstituted supernatant were injected for LC-UV-MS analysis.

Results. Metabolite profiling of M3385 in human liver S9 with or without two different cofactors NADPH and UDPGA (Fonsi et al., “High-Throughput Microsomal Stability Assay for Screening New Chemical Entities in Drug Discovery,” Journal of Biomolecular Screening 13(9):862-869 (2008), which is incorporated by reference herein in its entirety) showed that 88.6% of M3385 was still intact after 4 hours incubation. No EXT (payload) but 3.9% ProDXd and 3.5% DXd were detected (including both E-ring open and close forms as shown in Schemes 17-19).

A summary, including observed m/z value, retention time and MS peak area of model compound M3385 and its metabolites in human liver S9 with NADPH and UDPGA are presented in Tables 14 and 15 below.

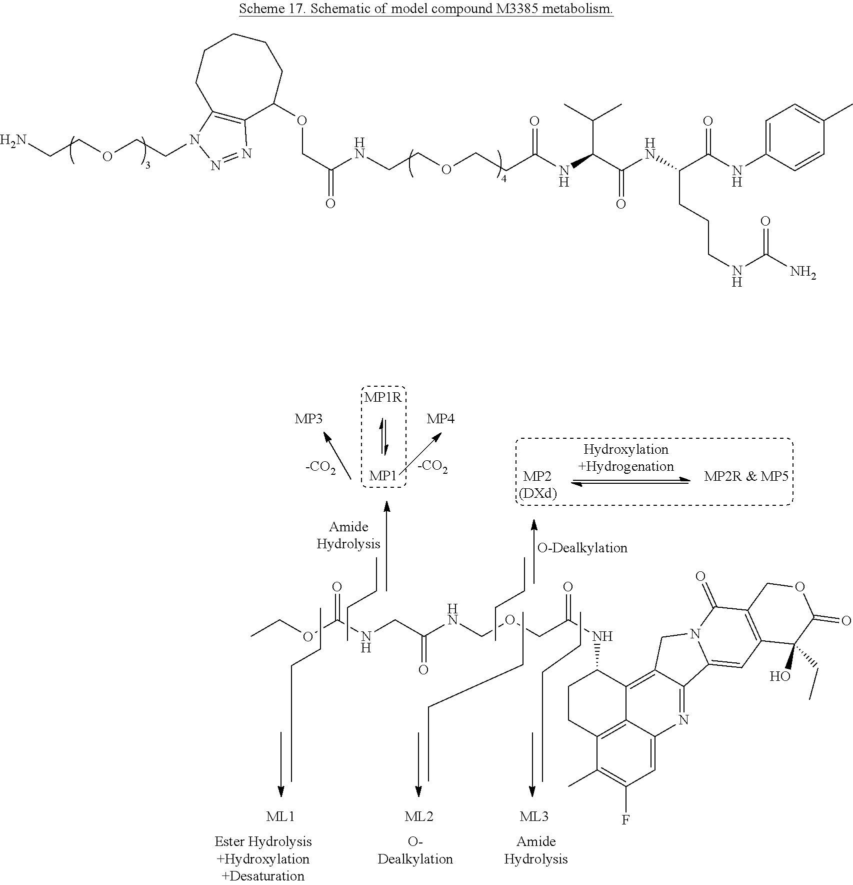

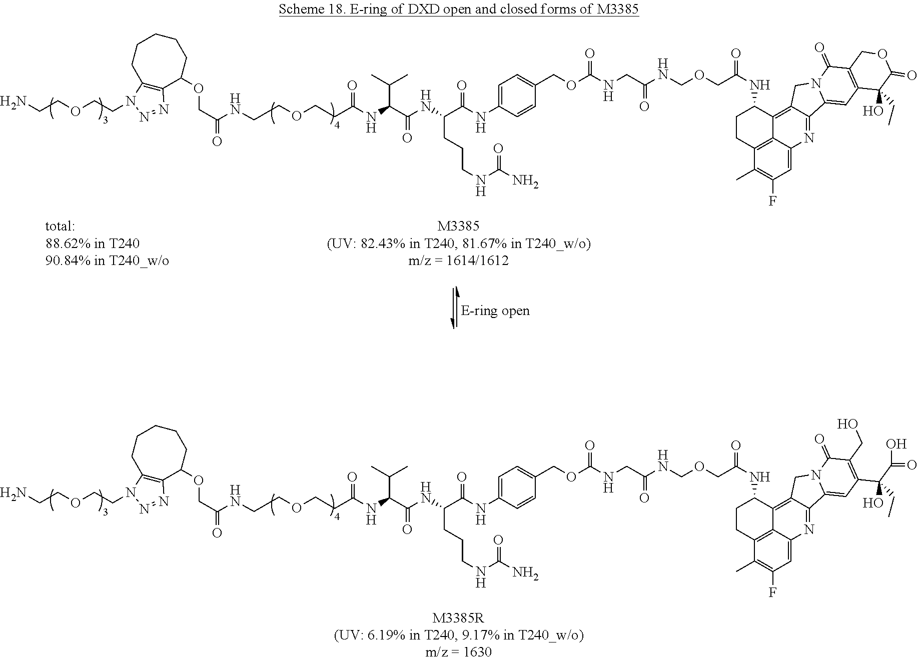

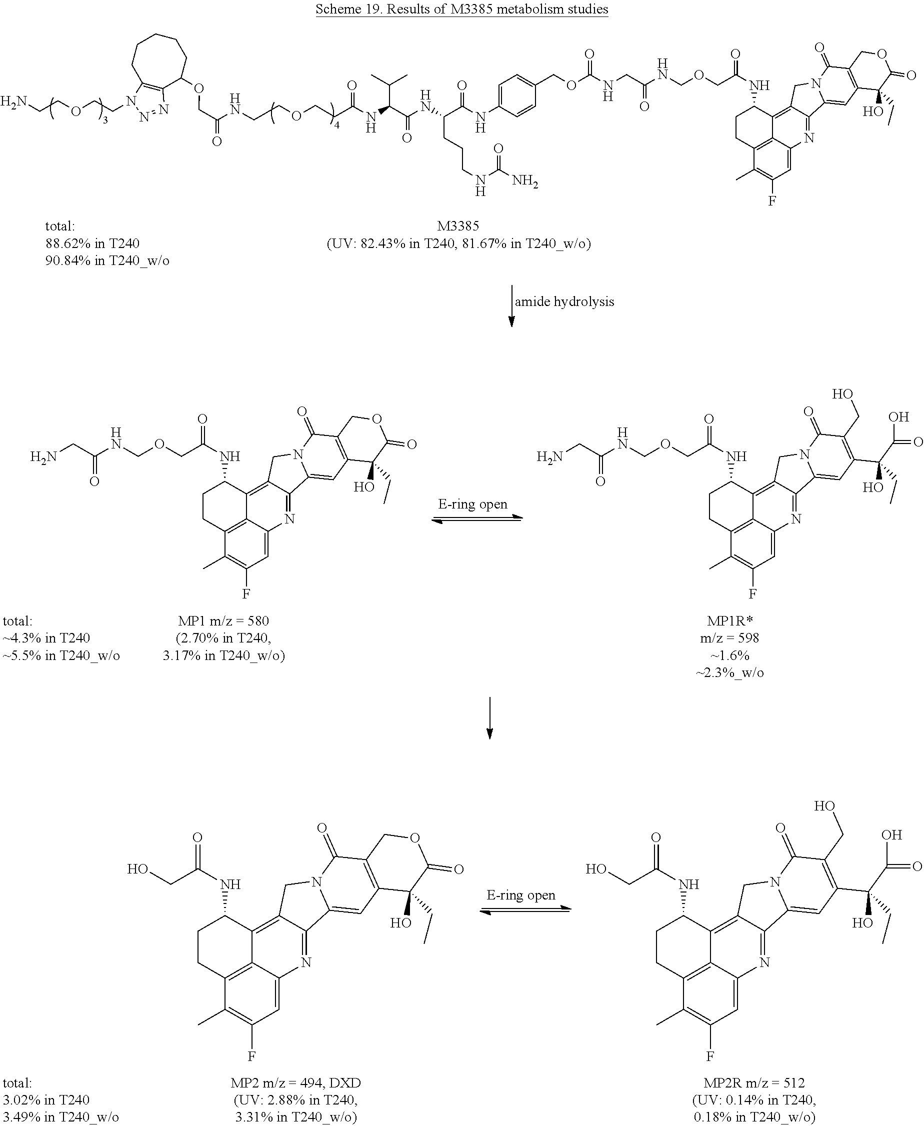

TABLE 14
M3385 Metabolism in Human Liver S9 with cofactors NADPH and UDPGA (240 min)

| | | | | Human Liver S9 with NADPH | | |
|---|---|---|---|---|---|---|
| | | | | and UDPGA (240 min) | | |
| | | | | Relative UV | | |
| | | | R.T. | Abundance* | | MS peak |
| Peak ID | Found m/z | Biotransformation | (min) | (%) | | area |
| Parent(T240) | 1614.7884[M + H]⁺ | E-ring close form | 5.66 | 82.43% | 88.62% | 1.01E+06 |
| M3385 | 807.8961[M + 2H]²⁺ | | | | M3385 | 8.70E+06 |
| | 1612.7701[M − H]⁻ | | | | | 4.19E+05 |
| M3385R (E- | 816.9016[M + 2H]²⁺ | Hydroxylation + | 5.60 | 6.19% | | 2.02E+05 |
| ring open) | 1630.7828[M − H]⁻ | Hydrogenation | 5.60 | | | 1.10E+05 |
| | | E-ring open form | | | | |
| ML1 | 1023.5715[M + H]⁺ | Ester Hydrolysis + | 4.51 | + | + | 1.65E+04 |
| | | Hydroxylation + | | | | |
| | | Desaturation | | | | |
| MP1 | 580.2186[M + H]⁺ | Amide Hydrolysis | 4.56 | 2.70% | 3.94% | 2.45E+05 |
| MP1R | 598.2317[M + H]⁺ | E-ring open form | 4.61 | 0.29% | ProDXd | 3.98E+04 |
| | | from MP1 | | | | |
| MP4 | 534.2162[M − H]⁻ | MP1 + (—CO2) | 4.62 | 0.95% | | 1.31E+05 |
| MP3 | 550.2102[M − H]⁻ | MP1 + (—CO) | 4.63 | + | | 2.44E+04 |
| ML2 | + 1139.6295[M + H]⁺ | O-Dealkylation | 4.34 | + | + | 1.17E+05 |
| MP2 | 494.1710[M + H]⁺ | O-Dealkylation | 5.39 | 2.88% | 3.45% | 5.97E+05 |
| | | (DXd) | | | Dxd | |
| MP2R | 512.1829[M + H]⁺ | (E-ring open form | 5.19 | 0.14% | | 1.39E+04 |
| | | from DXd) | | | | |
| MP5 | 512.1816[M + H]⁺ | MP2 + | 4.61 | 0.43% | | 5.84E+04 |
| | | Hydroxylation + | | | | |
| | | Hydrogenation | | | | |
| ML3 | 1195.6258[M − H]⁻ | Amide Hydrolysis | 4.43 | + | + | 7.26E+03 |

TABLE 15
M3385 Metabolism in Human Liver S9 Without Cofactors NADPH and UDPGA
(240 min)

| | | | | Human Liver S9 without NADPH | | |
|---|---|---|---|---|---|---|
| | | | | and UDPGA (240 min) | | |
| | | | R.T. | Relative UV | | MS peak |
| Peak ID | Found m/z | Biotransformation | (min) | Abundance* (%) | | area |
| Parent(T240) | 1614.7884[M + H]⁺ | E-ring close form | 5.66 | 81.67% | 90.84% | 9.02E+05 |
| M3385 | 807.8961[M + 2H]²⁺ | | | | | 8.79E+06 |
| | 1612.7701[M − H]⁻ | | | | | 4.64E+05 |
| M3385R (E- | 816.9016[M + 2H]²⁺ | Hydroxylation + | 5.60 | 9.17% | | 3.39E+05 |
| ring open) | 1630.7828[M − H]⁻ | Hydrogenation | 5.60 | | | 1.87E+05 |
| | | E-ring open form | | | | |
| ML1 | 1023.5715[M + H]⁺ | Ester Hydrolysis + | 4.51 | + | + | 2.23E+04 |
| | | Hydroxylation + | | | | |
| | | Desaturation | | | | |
| MP1 | 580.2186[M + H]⁺ | Amide Hydrolysis | 4.56 | 3.17% | 4.93% | 3.00E+05 |
| MP1R | 598.2317[M + H]⁺ | E-ring open form | 4.61 | 0.38% | | 5.08E+04 |
| | | from MP1 | | | | |
| MP4 | 534.2162[M − H]⁻ | MP1 + (—CO2) | 4.62 | 1.38% | | 1.81E+05 |
| MP3 | 550.2102[M − H]⁻ | MP1 + (—CO) | 4.63 | + | | 3.45E+04 |
| ML2 | 1139.6295[M + H]⁺ | O-Dealkylation | 4.34 | + | + | 1.21E+05 |
| MP2 | 494.1710[M + H]⁺ | O-Dealkylation | 5.39 | 3.31% | | 7.60E+05 |
| | | (DXd) | | | | |
| MP2R | 512.1829[M + H]⁺ | (E-ring open form | 5.19 | 0.18% | 4.05% | 2.48E+04 |
| | | from DXd) | | | | |
| MP5 | 512.1816[M + H]⁺ | MP2 + | 4.61 | 0.56% | | 7.28E+04 |
| | | Hydroxylation + | | | | |
| | | Hydrogenation | | | | |
| ML3 | 1195.6258[M − H]⁻ | Amide Hydrolysis | 4.43 | + | + | 7.41E+03 |

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

1.-99. (canceled)

100. An antibody-drug conjugate comprising an antibody or an antigen-binding fragment thereof conjugated to a compound having Formula (I) 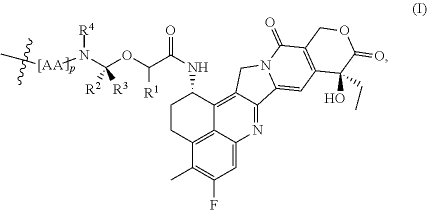
or a pharmaceutically acceptable salt thereof,
wherein R¹, R², R³, and R⁴ are independently hydrogen, a C₁₋₅ alkyl, or aryl;
AA is a natural or a nonnatural amino acid;
p is an integer from 1 to 6, and 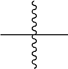
indicates the point of attachment to the antibody or the antigen-binding fragment thereof, directly or via a linker.

101. The antibody-drug conjugate according to claim 100, wherein said compound of Formula (I) comprises

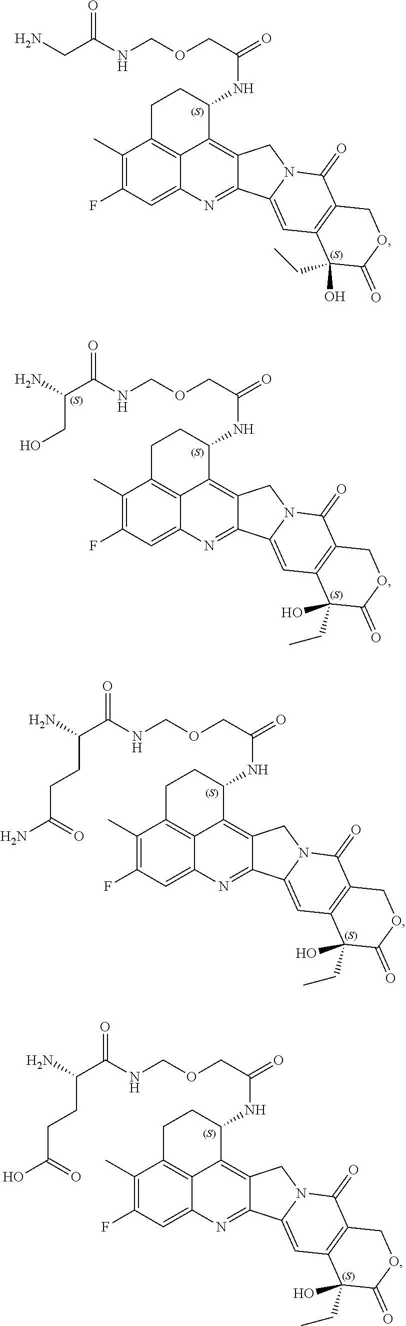

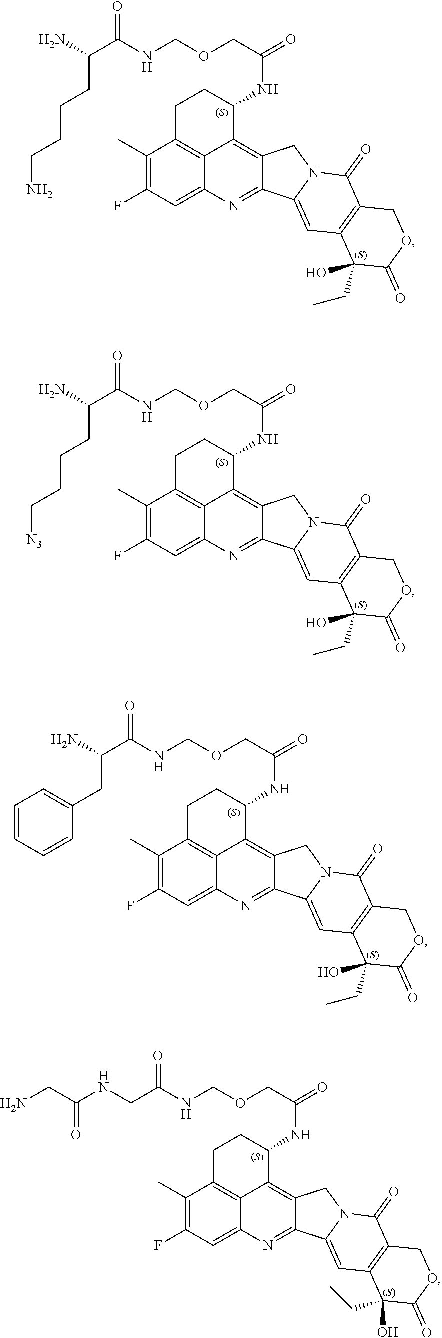

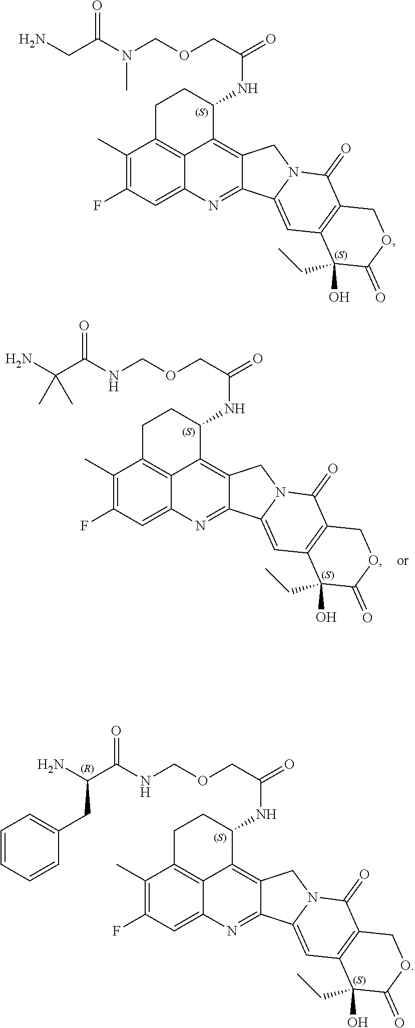

102. The antibody-drug conjugate according to claim 100, wherein said antibody or said antigen-binding fragment thereof is conjugated to a compound having a structure according to Formula (II) 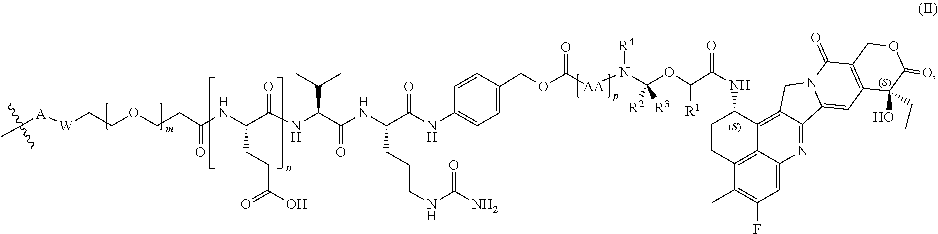
or a pharmaceutically acceptable salt thereof, wherein R¹, R², R³, and R⁴ are independently hydrogen or a C₁₋₅ alkyl;
A is a Click chemistry adduct;
W is NH, O, CO, CH₂, a phenyl, or a combination of two or more thereof,
AA is a natural or a nonnatural amino acid;
m is an integer from 0 to 8;
n is 0 or 1;
p is an integer from 1 to 6, and 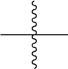
indicates the point of attachment to the antibody or the antigen-binding fragment thereof, directly or via a linker.

103. The antibody-drug conjugate according to claim 102, wherein the click chemistry adduct is a product of a copper-free click chemistry reaction selected from: (a) strain-promoted azide/dibenzocyclooctyne-amine (DBCO) click chemistry; (b) inverse electron demand Diels-Alder (IED-DA) tetrazine/trans-cyclooctene (TCO) click chemistry; (c) inverse electron demand Diels-Alder (IED-DA) tetrazine/norbonene click chemistry; (d) Diels-Alder maleimide/furan click-chemistry; (e) Staudinger ligation; and (f) nitrile-oxide/norbonene cycloaddition click chemistry.

104. The antibody-drug conjugate according to claim 102, wherein the click chemistry adduct comprises a triazole or a diazine.

105. The antibody-drug conjugate according to claim 102, wherein the click chemistry adduct is selected from the group consisting of: 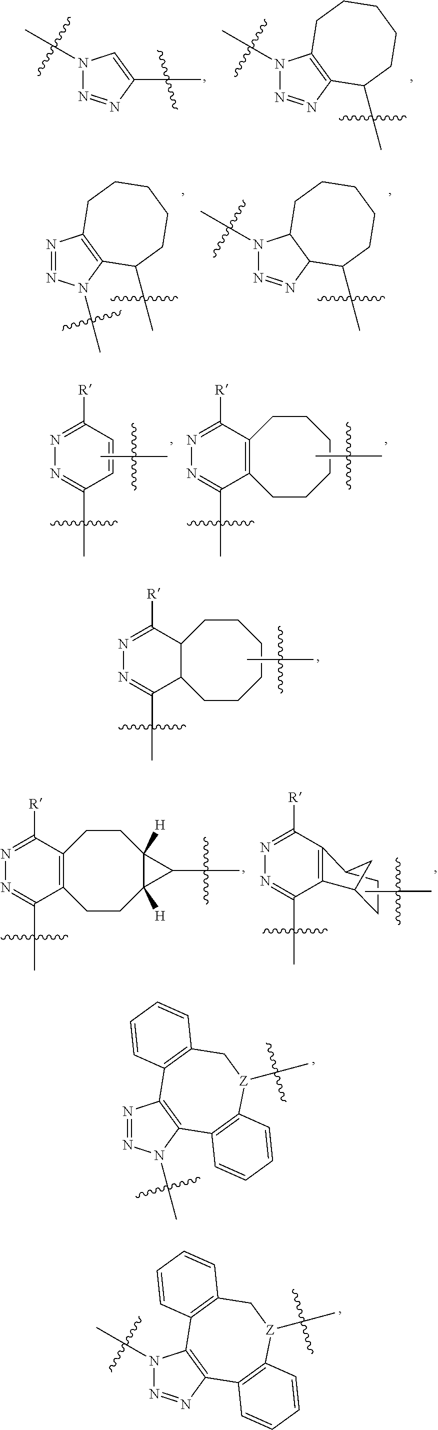
and any regio-isomers or entantiomers thereof, where R′ is H or a C₁₋₃ alkyl and Z is C or N.

106. The antibody-drug conjugate according to claim 102, wherein AA comprises a natural amino acid selected from glycine, alanine, valine, leucine, isoleucine, phenylalanine, tyrosine, threonine, lysine, asparagine, glutamine, aspartic acid, and glutamic acid.

107. The antibody-drug conjugate according to claim 102, wherein AA comprises a nonnatural amino acid selected from the group consisting of an R-amino acid, an N-methyl amino acid,

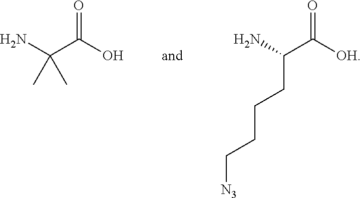

108. The antibody-drug conjugate according to claim 102, wherein said compound of Formula (II) comprises

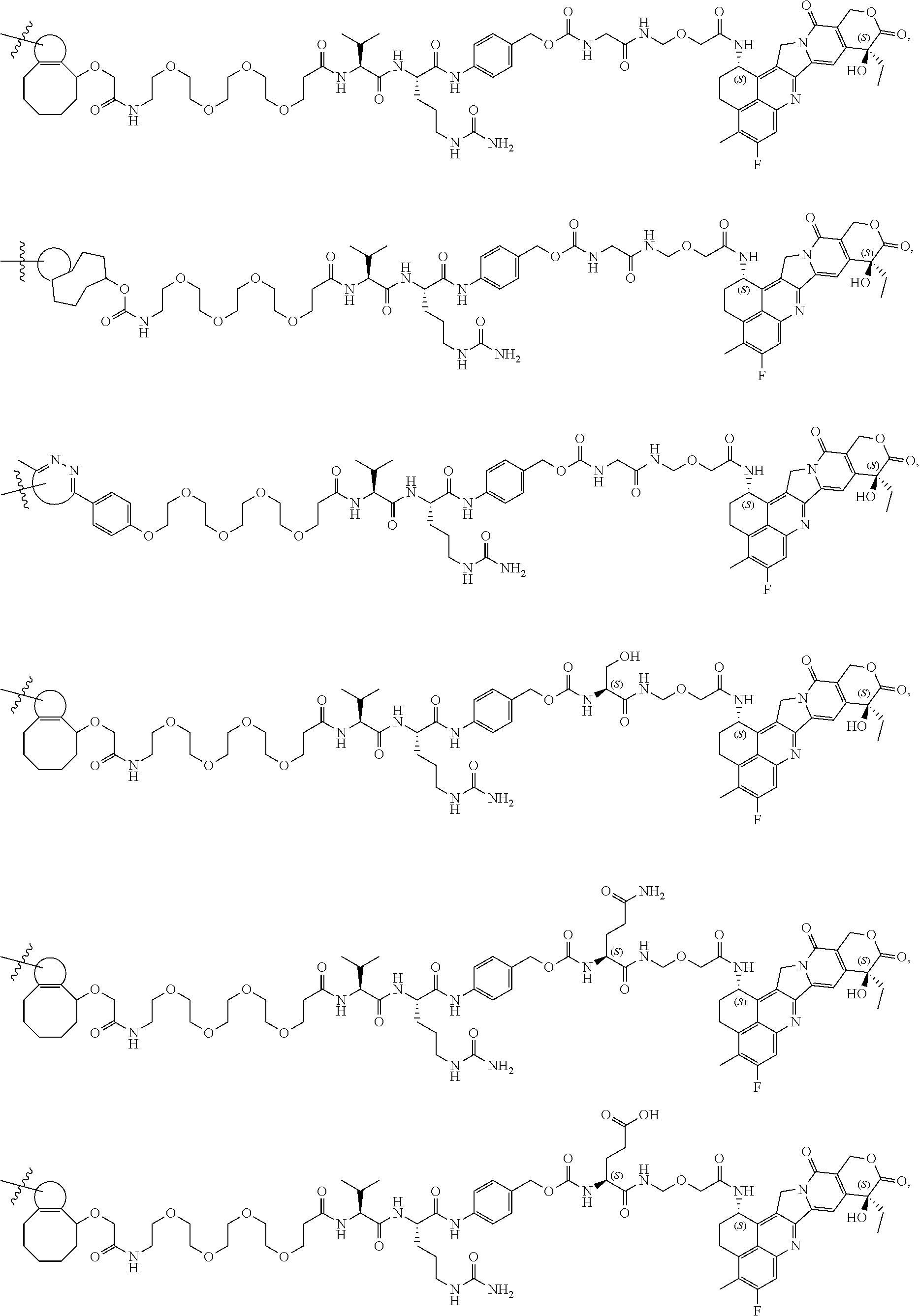

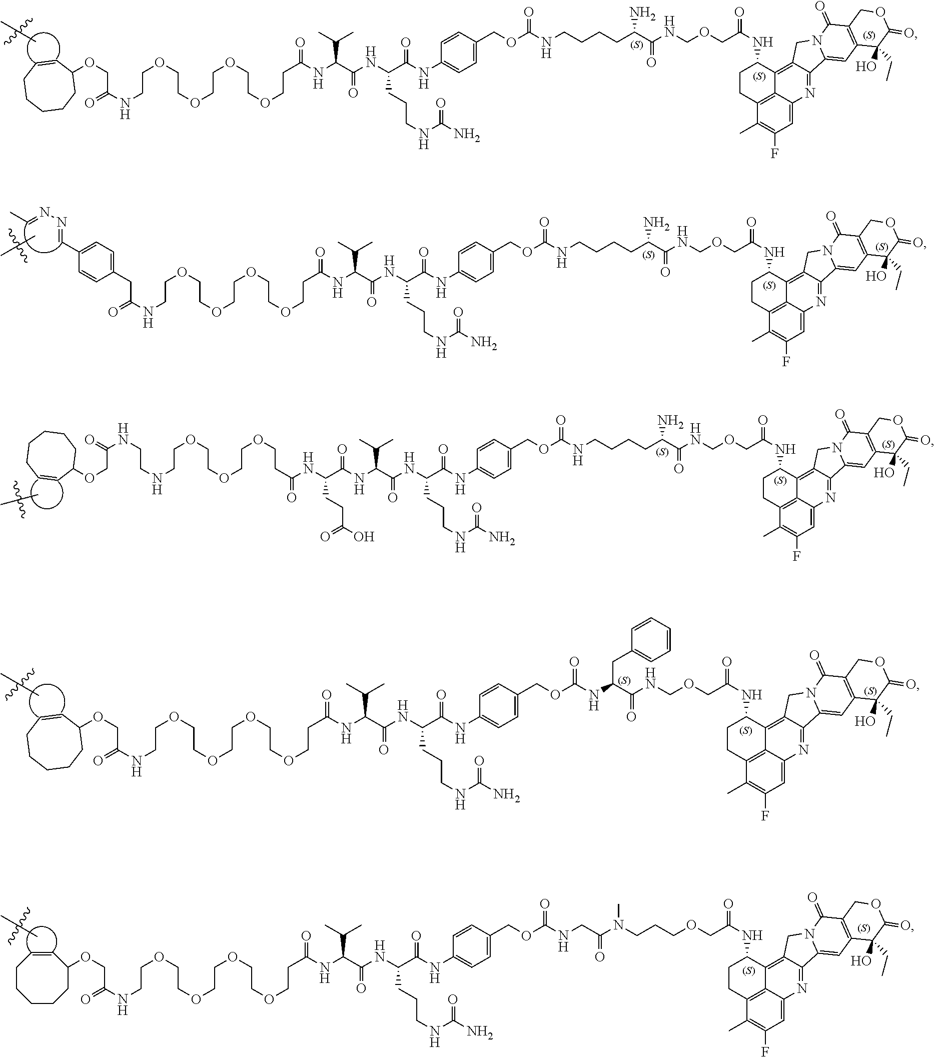

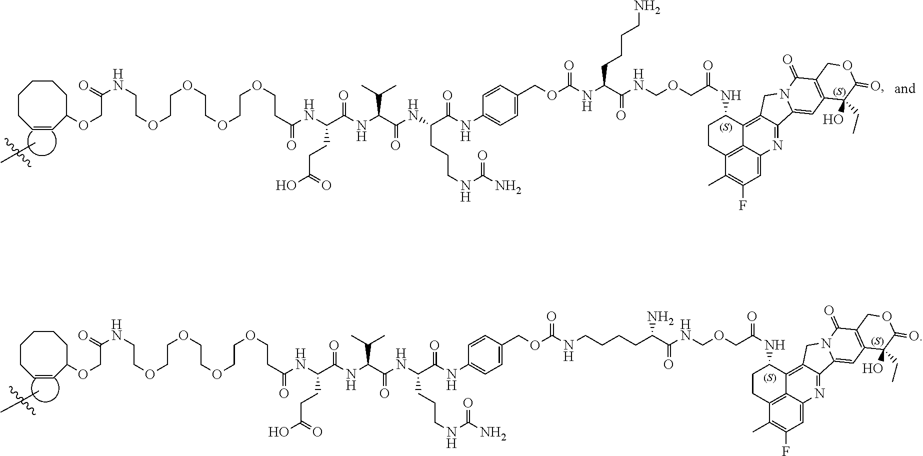

109. The antibody-drug conjugate according to claim 100 having a structure according to Formula (III) 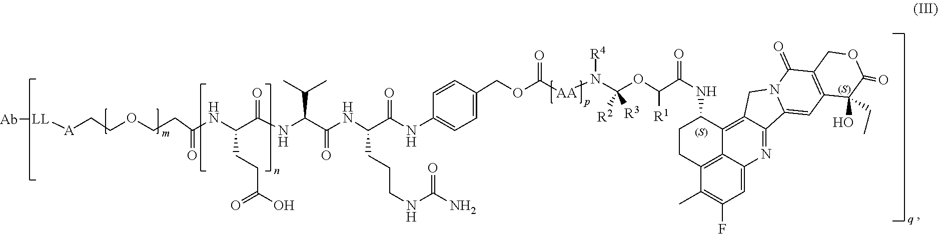
or a pharmaceutically acceptable salt thereof, wherein
Ab is an antibody or an antigen-binding fragment thereof,
R¹, R², R³, and R⁴ are independently hydrogen or a C₁₋₅ alkyl;
A is a Click chemistry adduct;
LL is a linker or a bond connecting said Ab and said A;
AA is a natural or a nonnatural amino acid;
m is an integer from 0 to 8;
n is 0 or 1;
p is an integer from 1 to 6;
and q is an integer from 1 to 10.

110. The antibody-drug conjugate according to claim 100 having a structure according to a Formula selected from the group consisting of IVa, IVb, IVc, IVd, IVe, IVf, IVg IVh, IVi, IVj, and IVk: 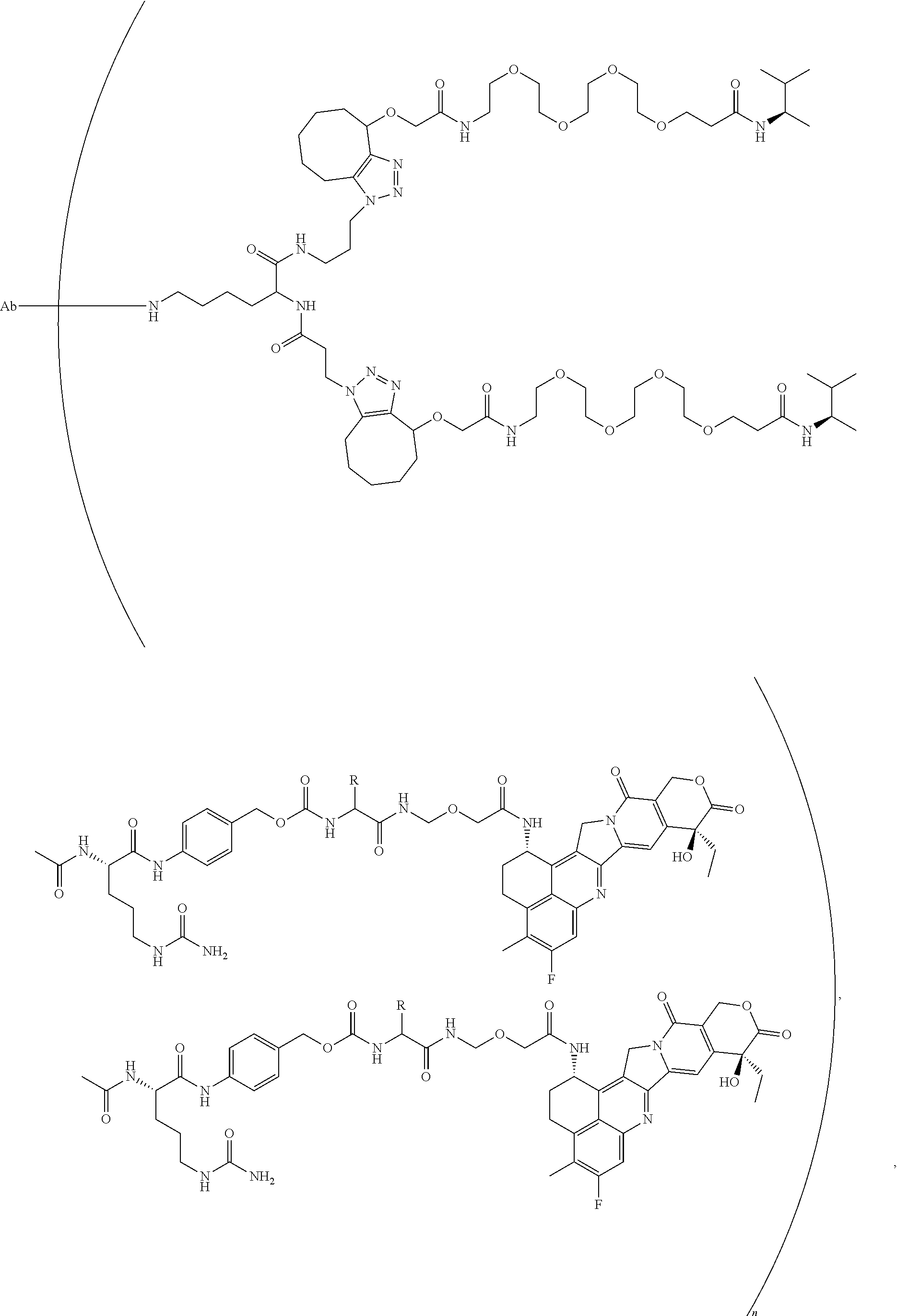 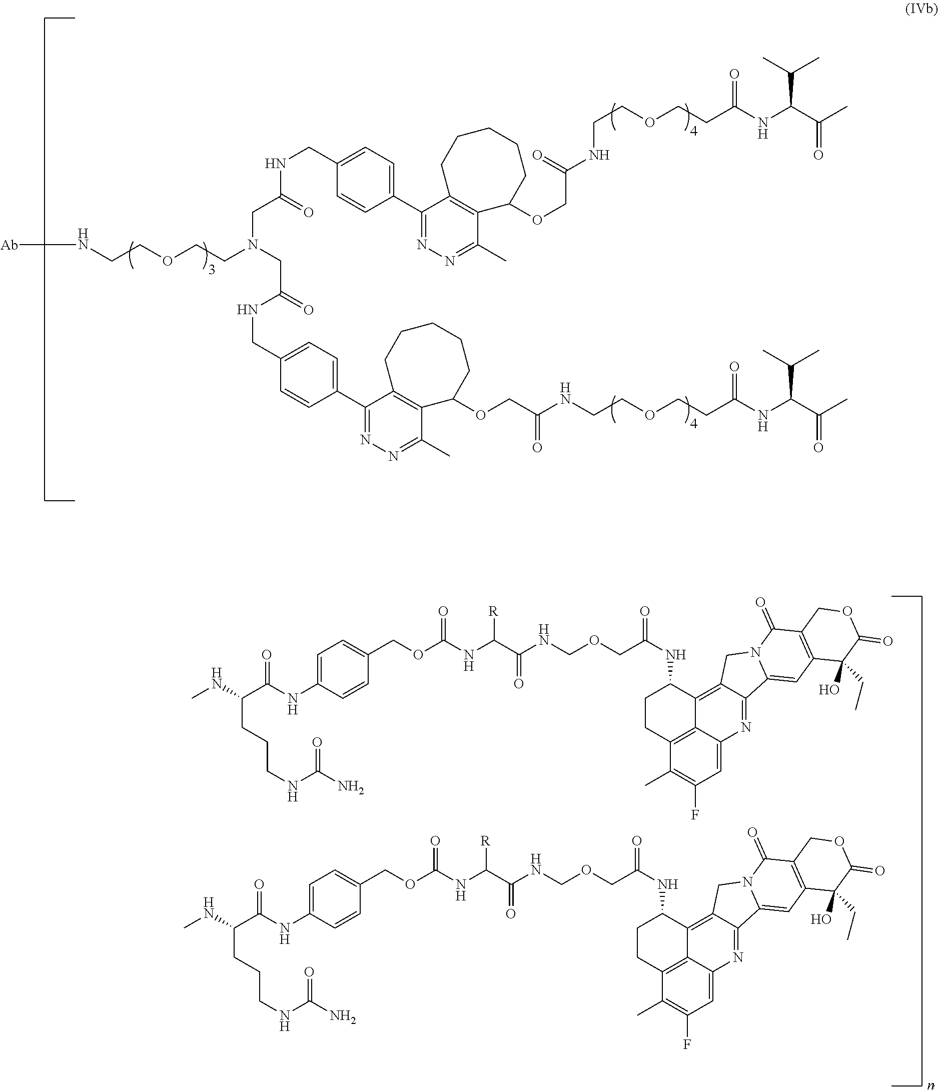 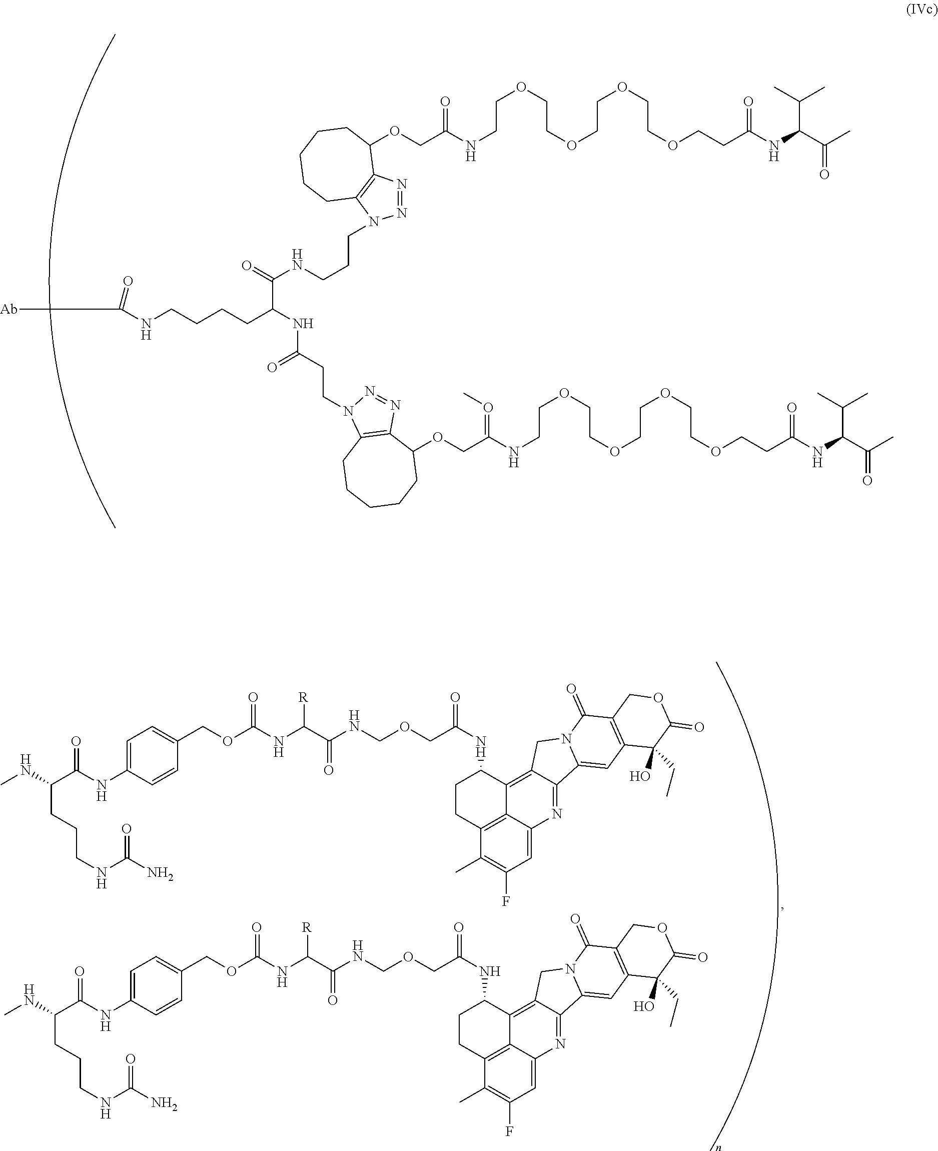 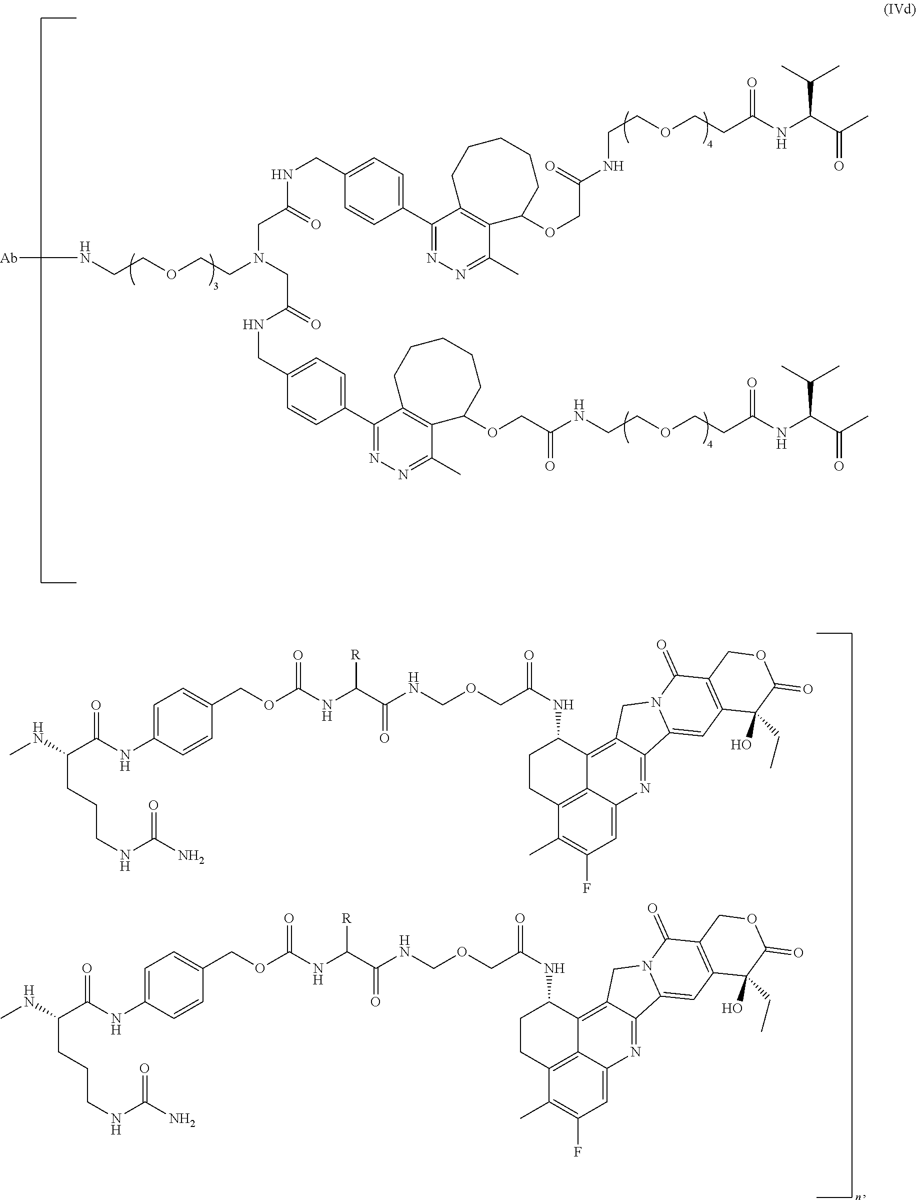 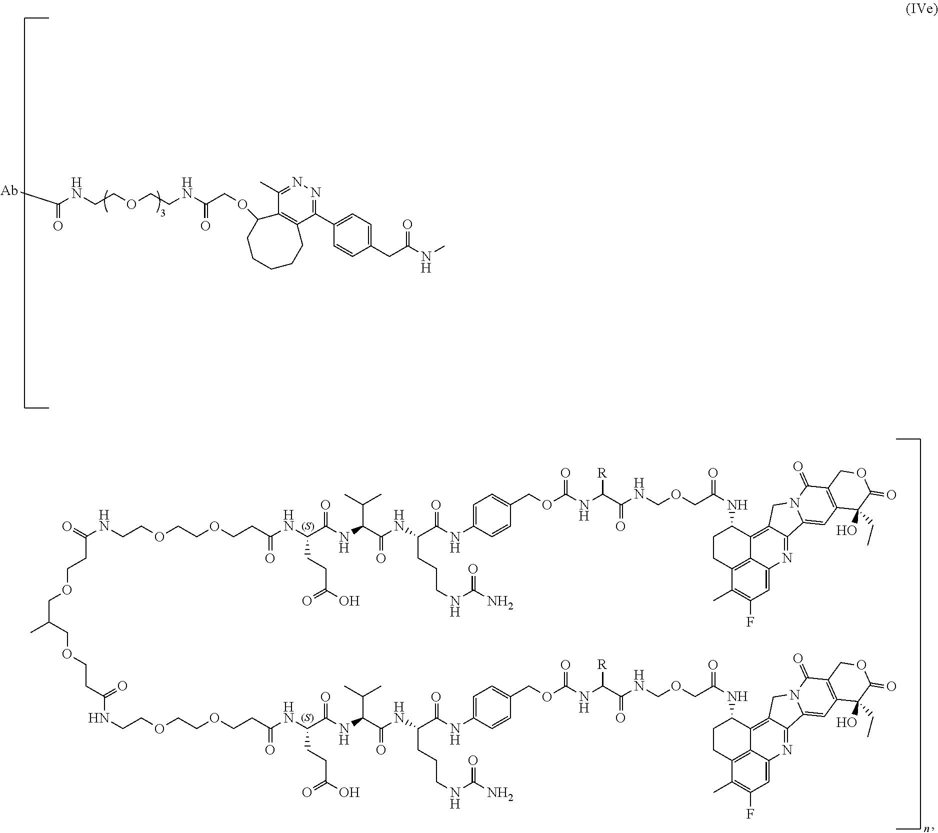 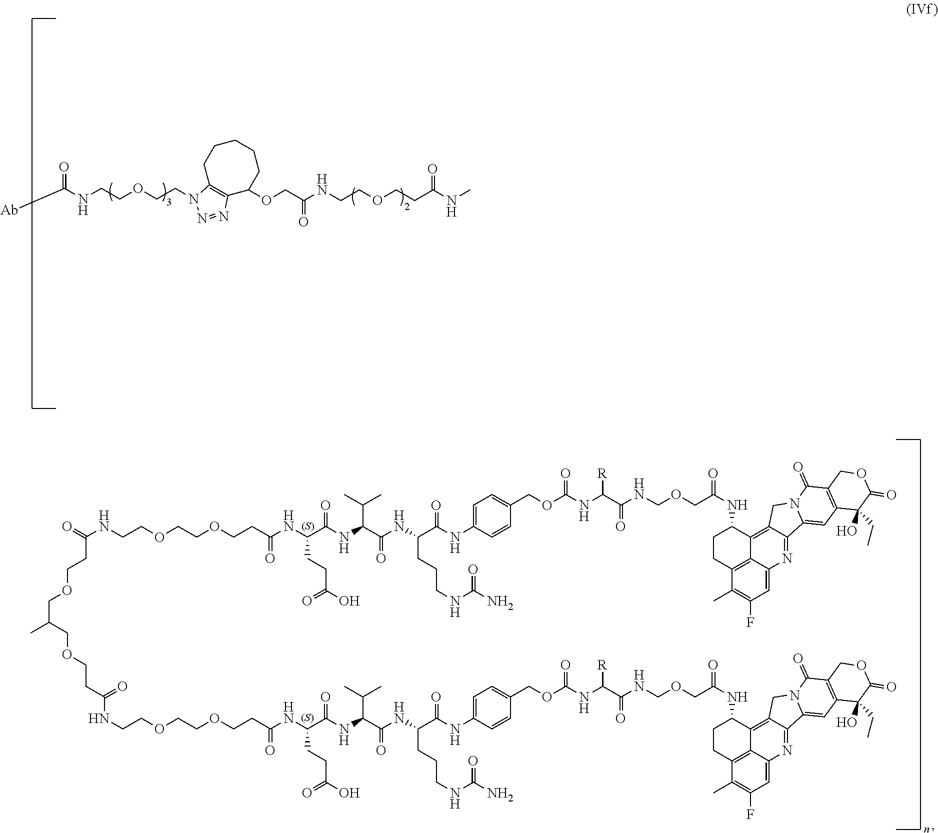 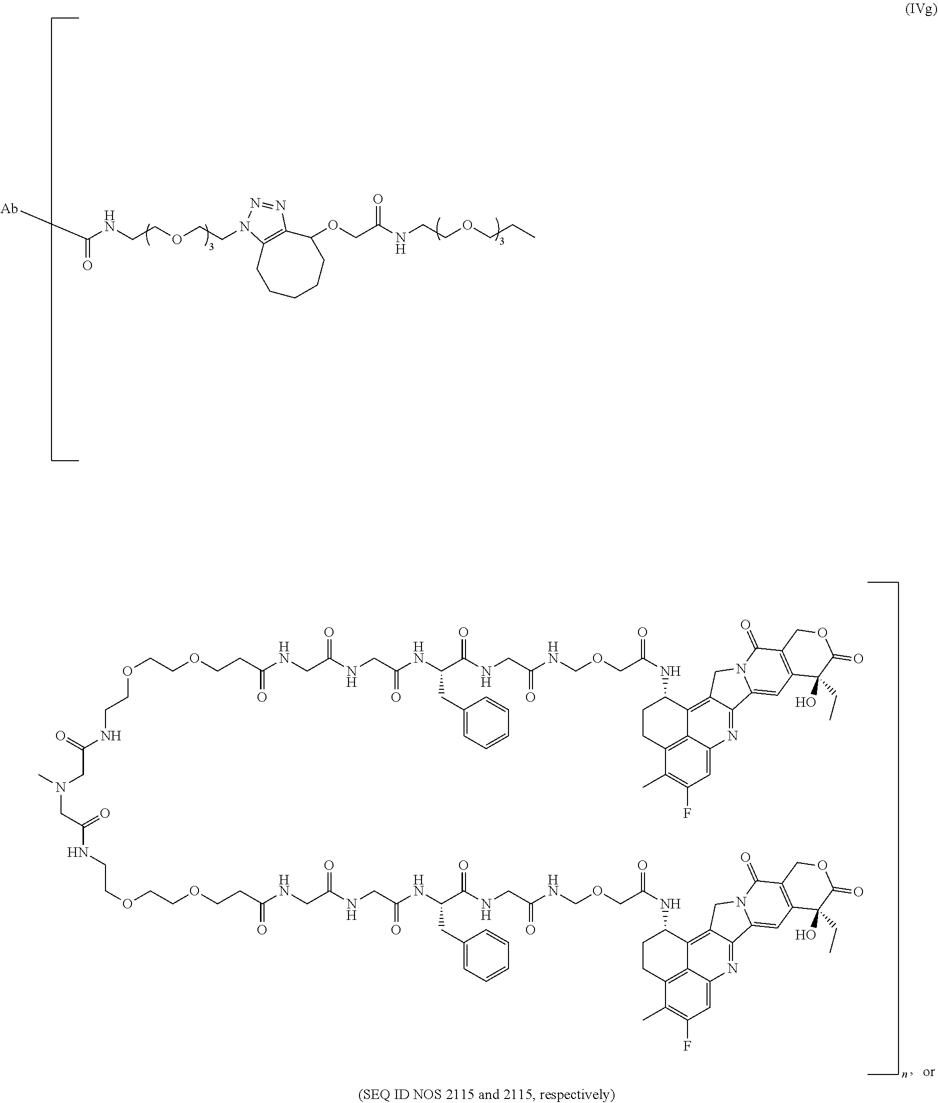 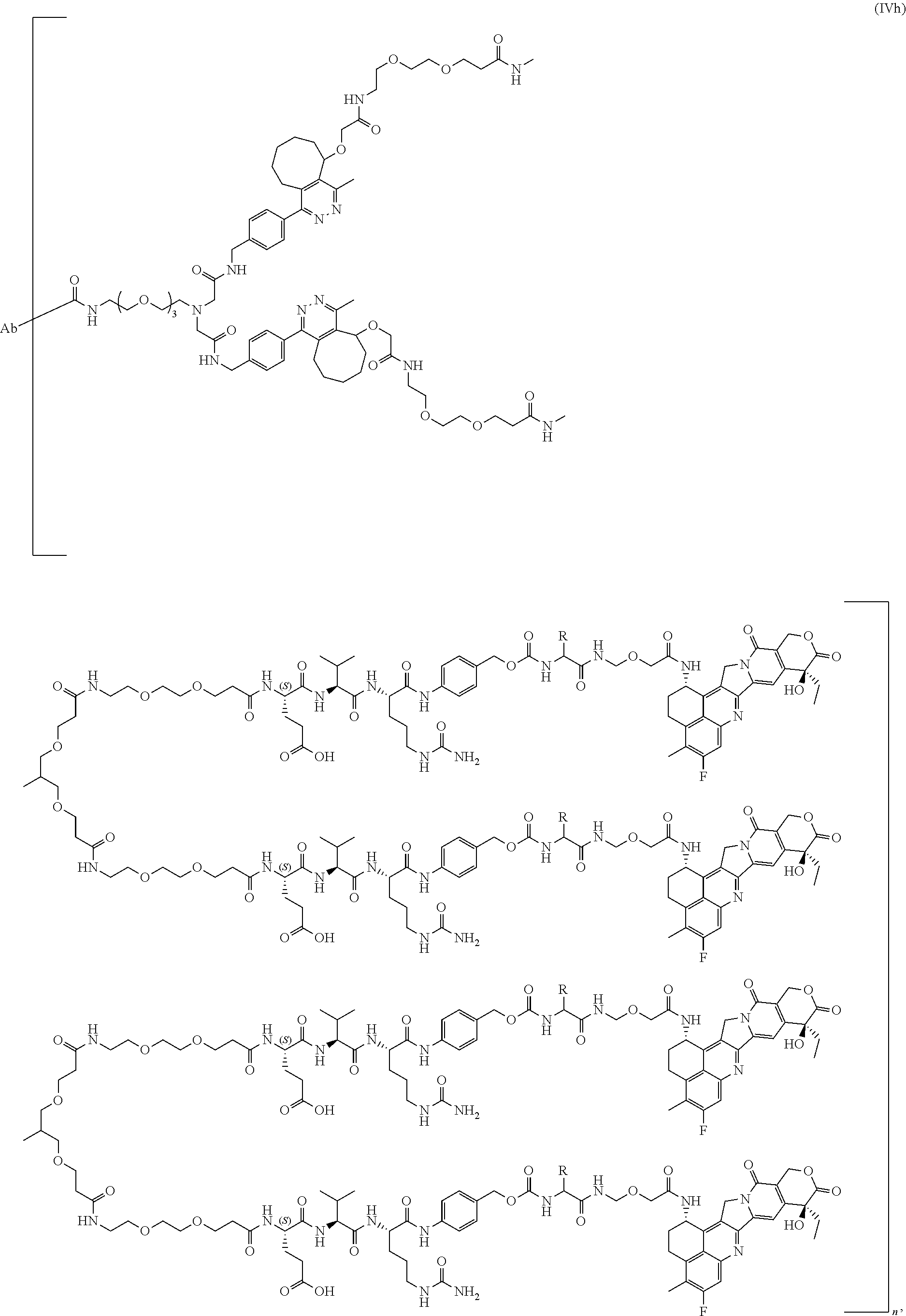 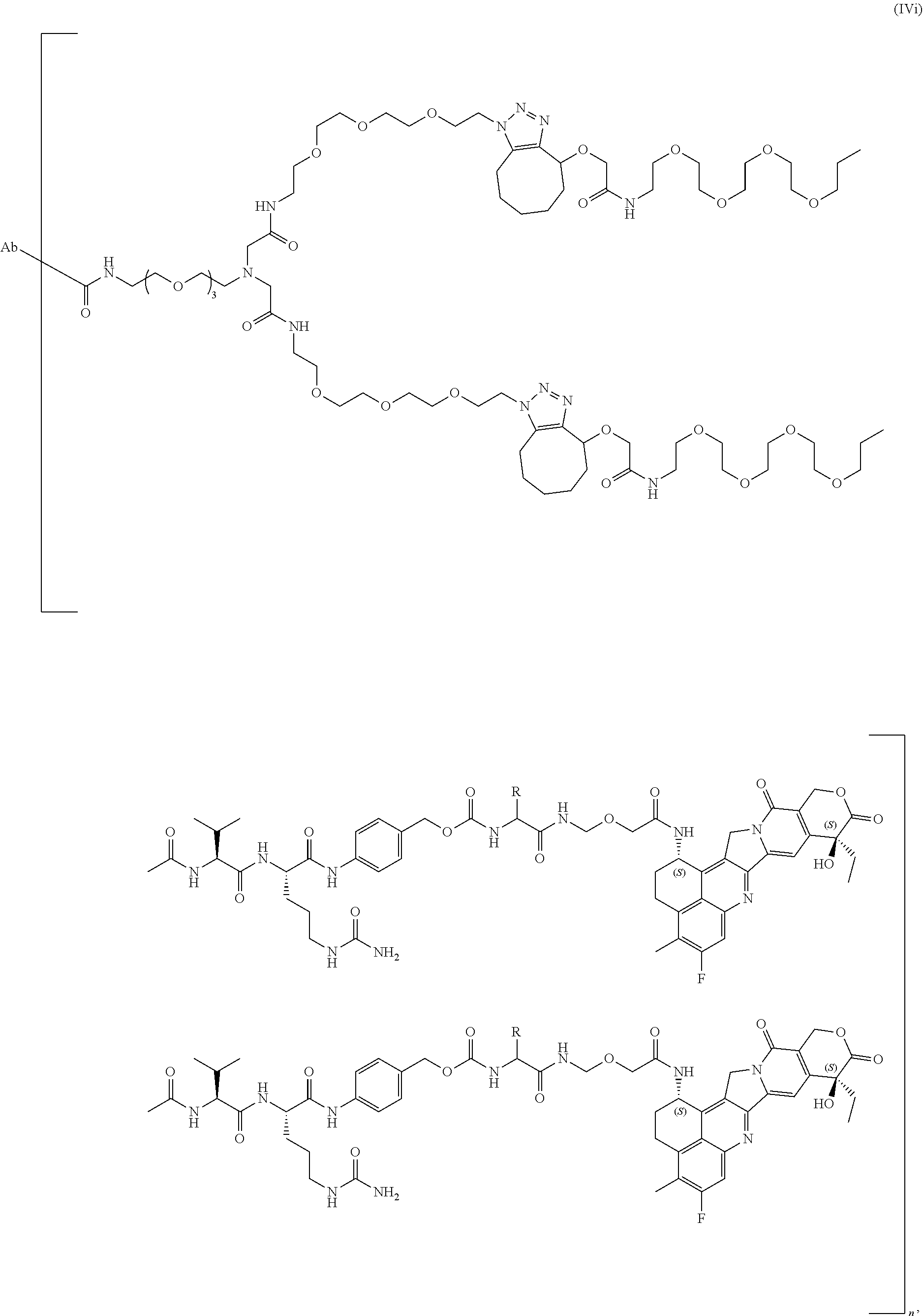 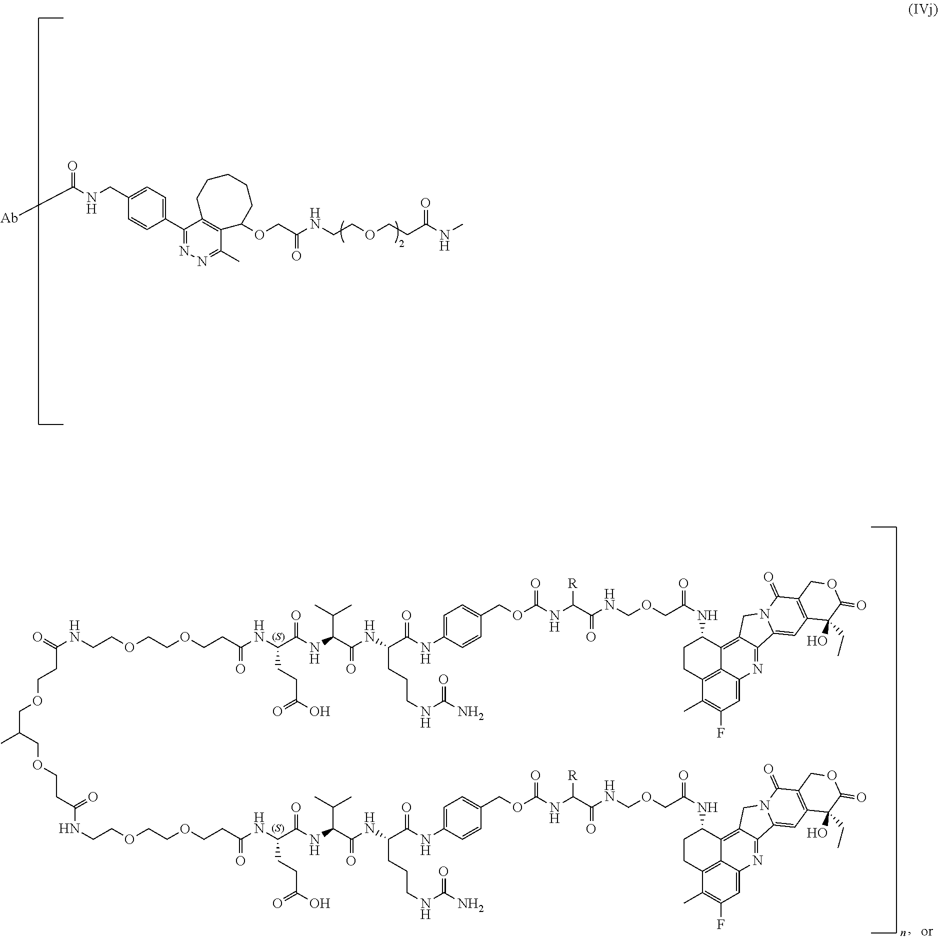 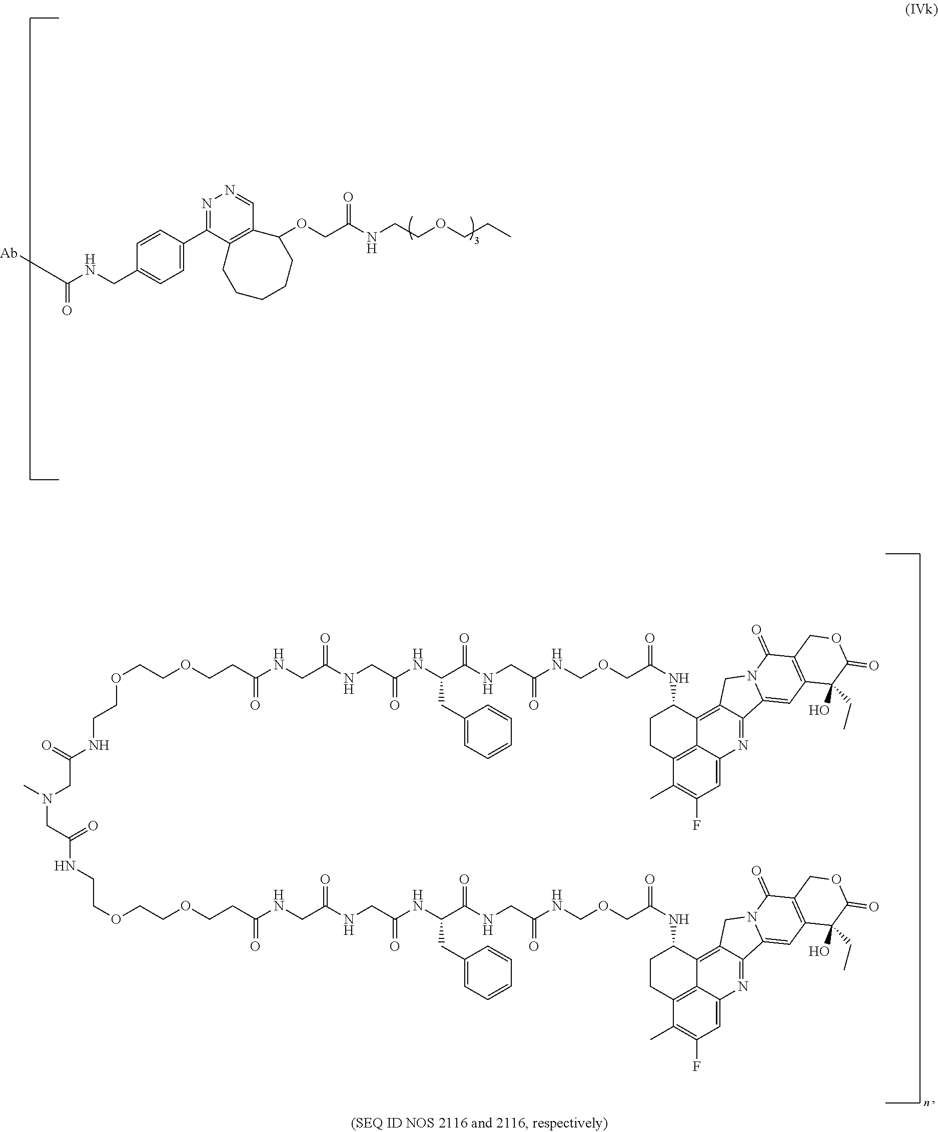
or a pharmaceutically acceptable salt thereof, wherein
Ab is an antibody or an antigen-binding fragment thereof,
R is a side chain of any natural or nonnatural amino acid;
and n is an integer from 1 to 5.

111. A pharmaceutical composition comprising an antibody-drug conjugate according to claim 100, co-formulated together with one or more pharmaceutically acceptable diluents, excipients, and/or additives.

112. A composition comprising a population of the antibody-drug conjugates according to claim 100, having a drug-antibody ratio (DAR) of about 0.5 to about 30.0.

113. A method for treating cancer in a subject in need thereof comprising the step of administering to the subject a therapeutically effective amount of the antibody-drug conjugate according to claim 100.

114. A linker-payload compound having the formula selected from the group consisting of (D′) to (N′): 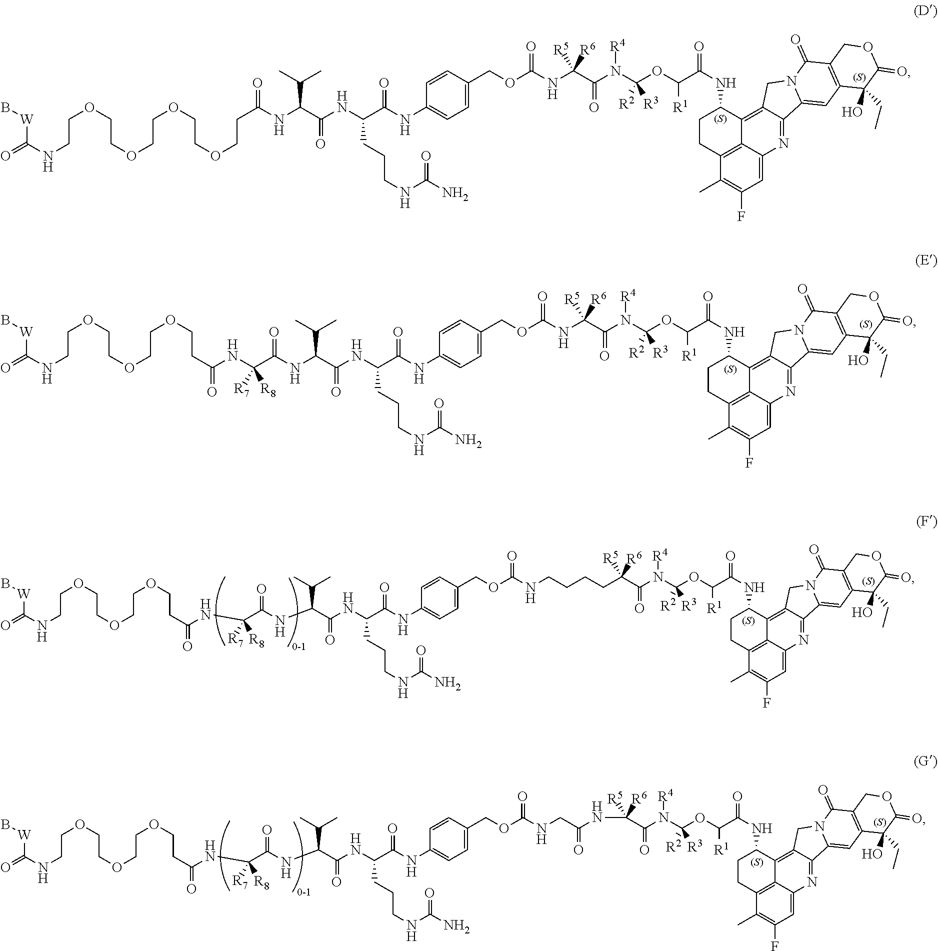 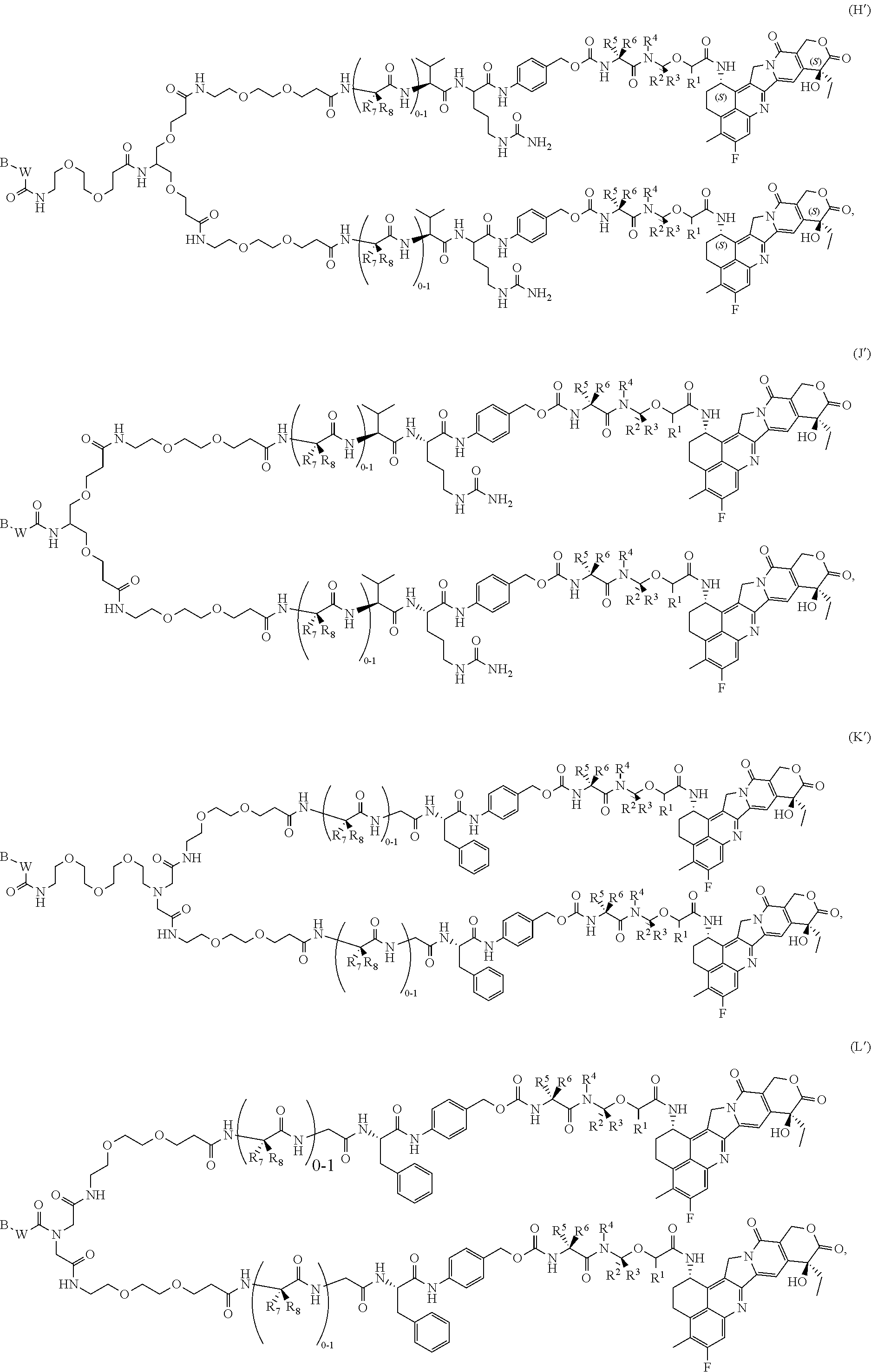 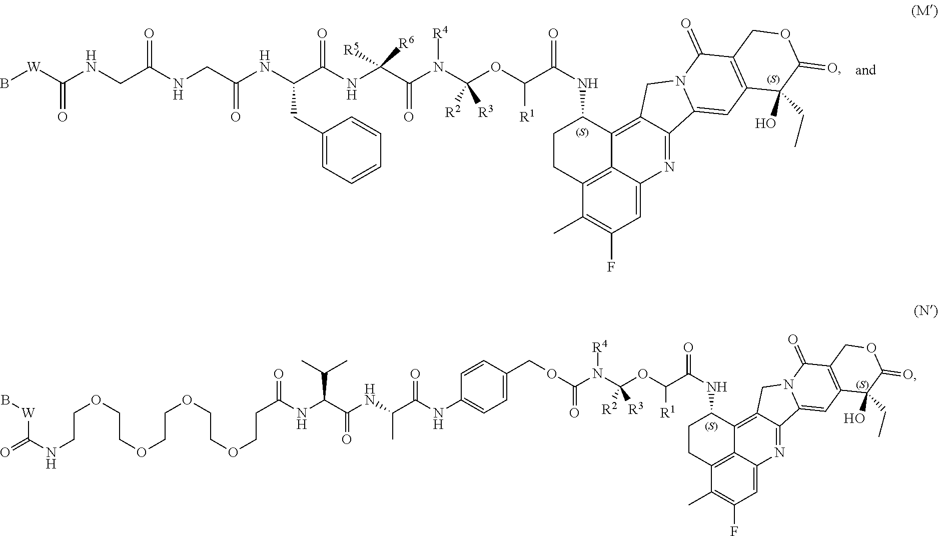
or a pharmaceutically acceptable salt thereof,
wherein R¹, R², R³, and R⁴ are independently hydrogen or a C₁₋₅ alkyl;
B is selected from the group consisting of 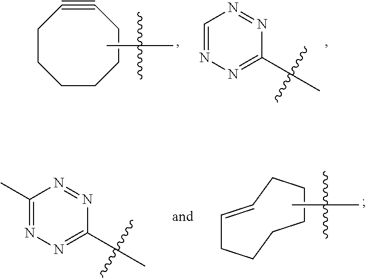
W is NH, O, CO, CH₂, a phenyl, or a combination of two or more thereof, and
R⁵, R⁶, R⁷ and R⁸ are independently hydrogen, —NH₂, or a side chain of any natural or non-natural amino acid,
the method comprising a step of exposing a payload having an amino group to an activated intermediate having a para-nitro-phenyl carbonate in the presence of a base and a coupling catalyst to afford said linker-payload compound (D′)-(G′), wherein said coupling catalyst is 4-Hydroxy-2-methylquinoline (MeHYQ).

115. The linker-payload compound of claim 114 having the structure of formula (D), 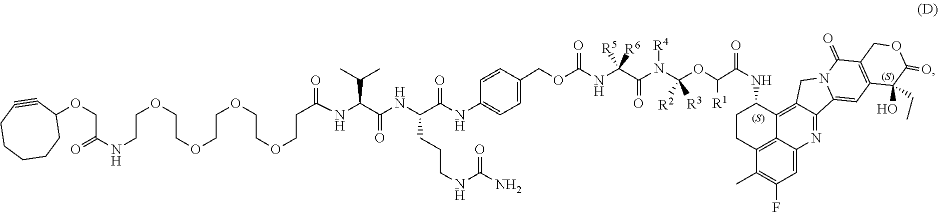
or a pharmaceutically acceptable salt thereof,
wherein R¹, R², R³, and R⁴ are independently hydrogen or a C₁₋₅ alkyl, and
R⁵ and R⁶ are independently hydrogen, —NH₂, or a side chain of any natural or non-natural amino acid.

116. The linker-payload compound of claim 114 having the structure selected from the group consisting of:

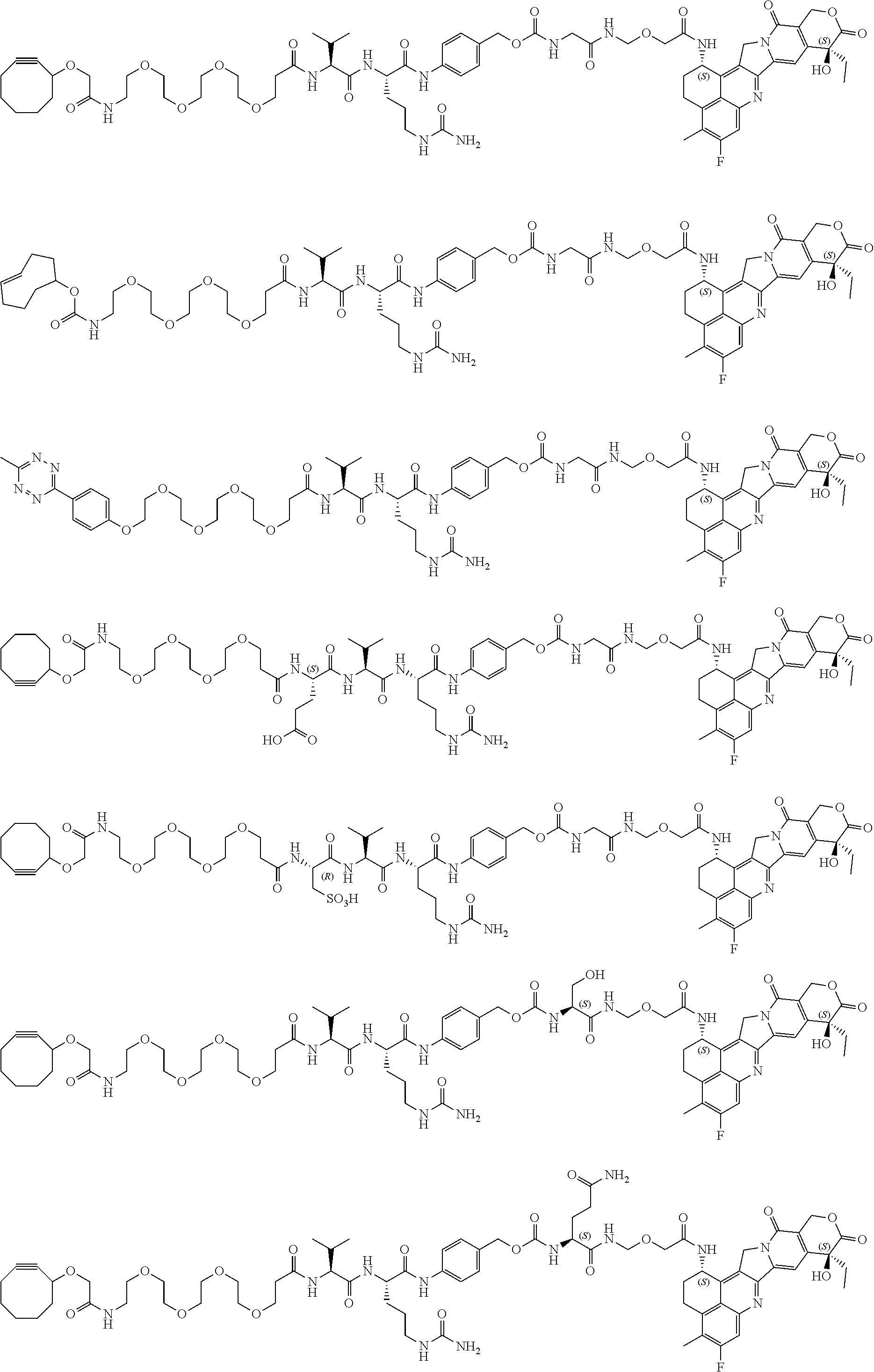

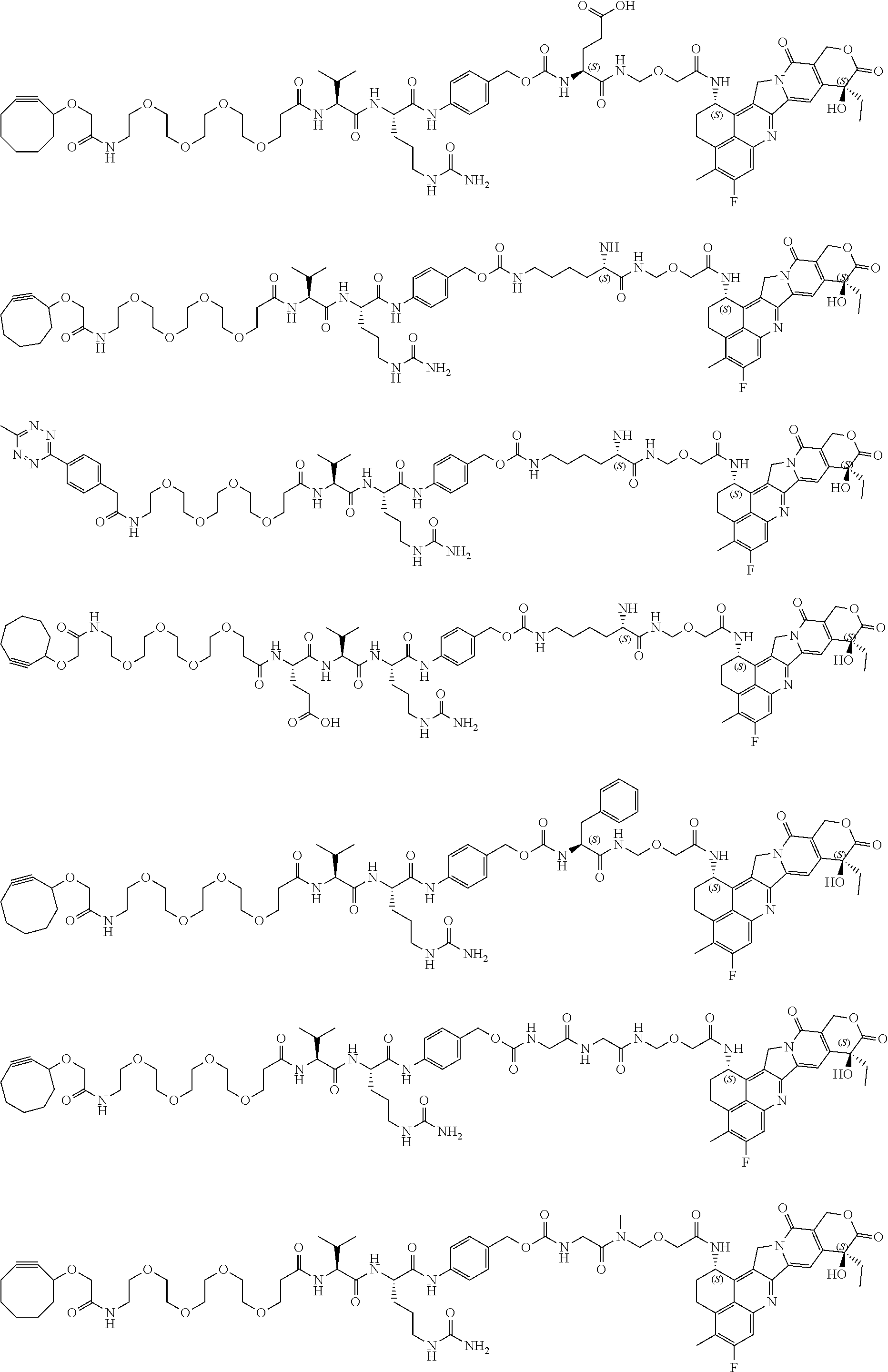

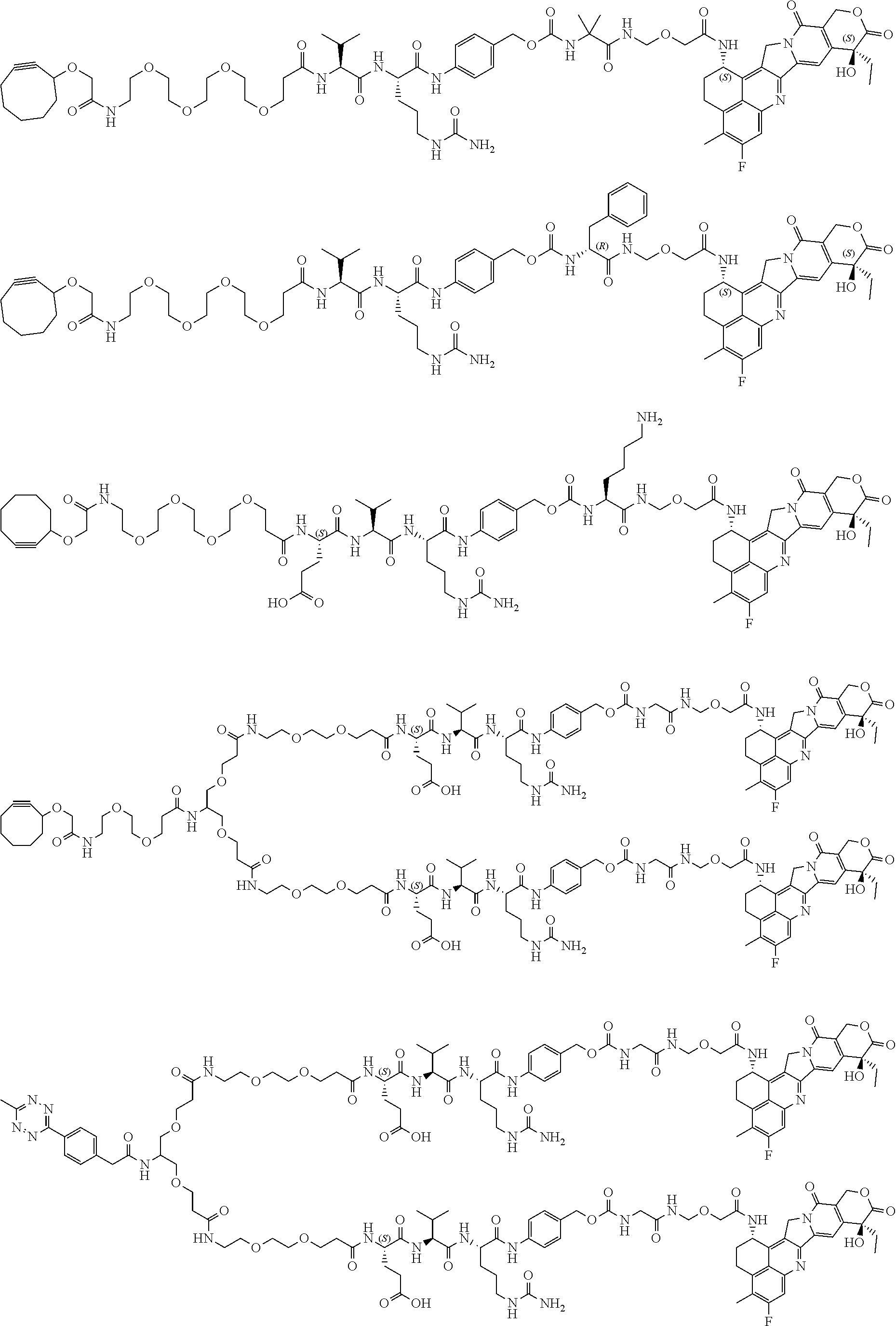

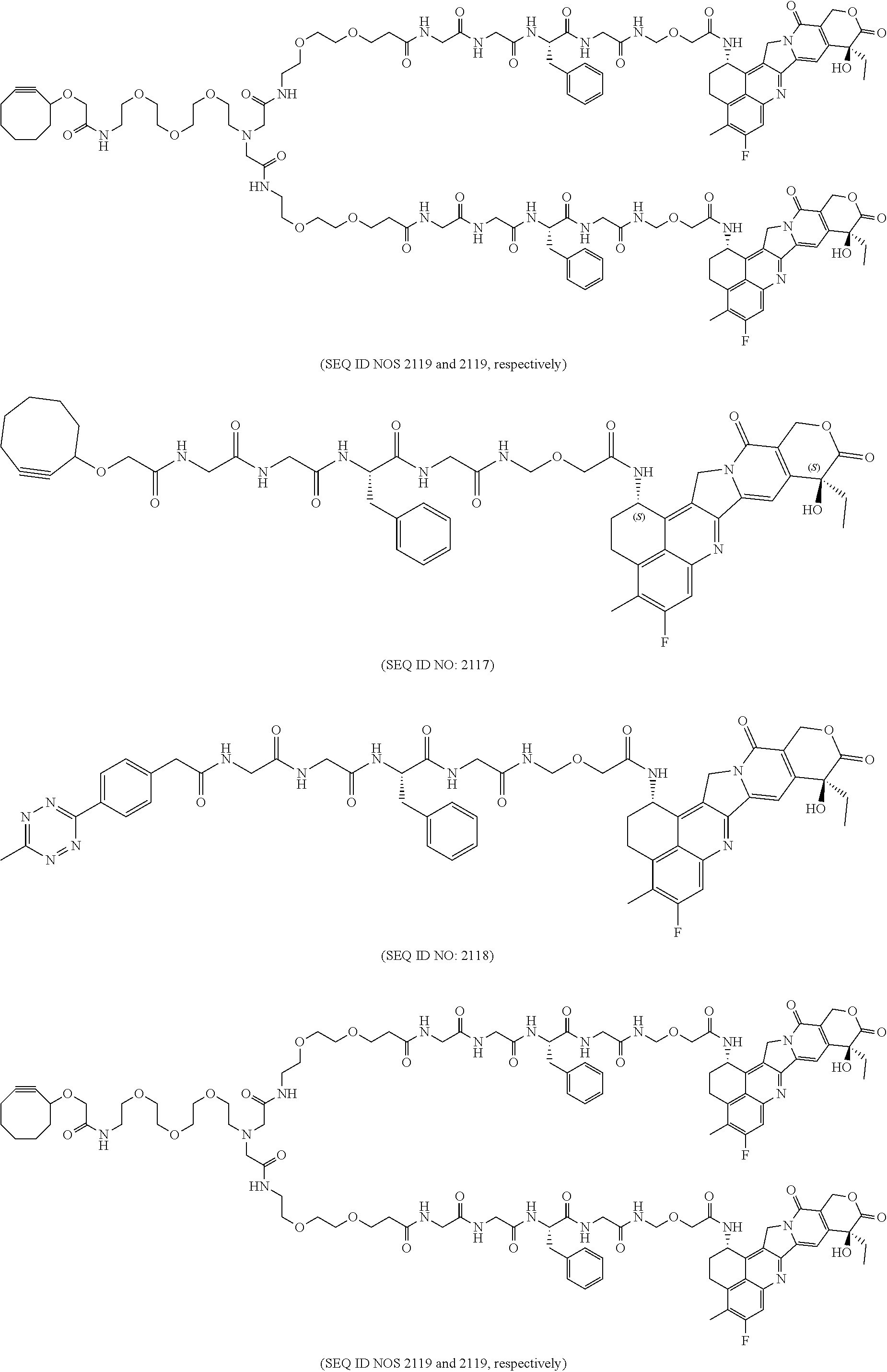

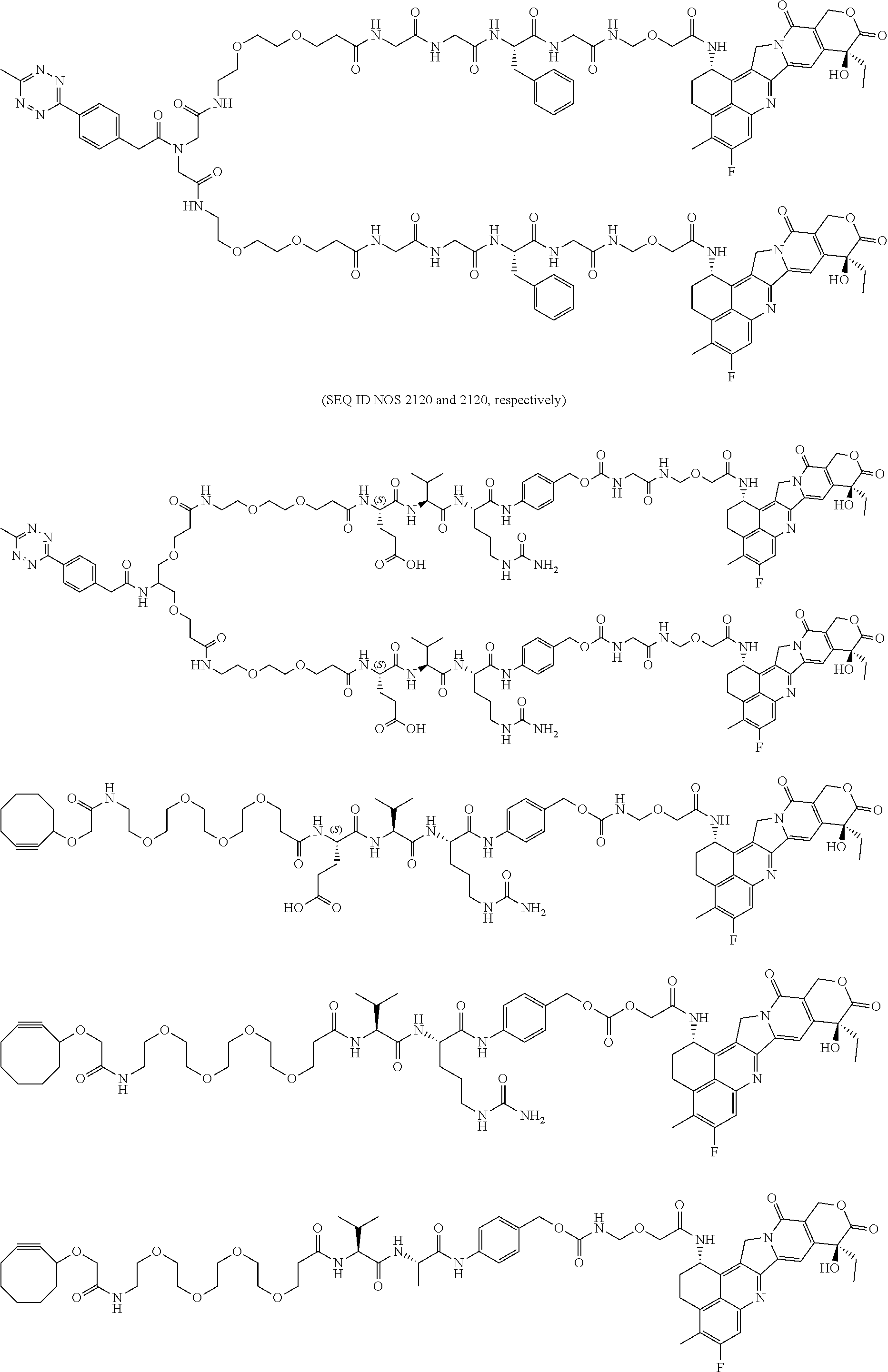

117. A process for manufacturing a linker-payload compound having the formula selected from the group consisting of (D′) to (N′): 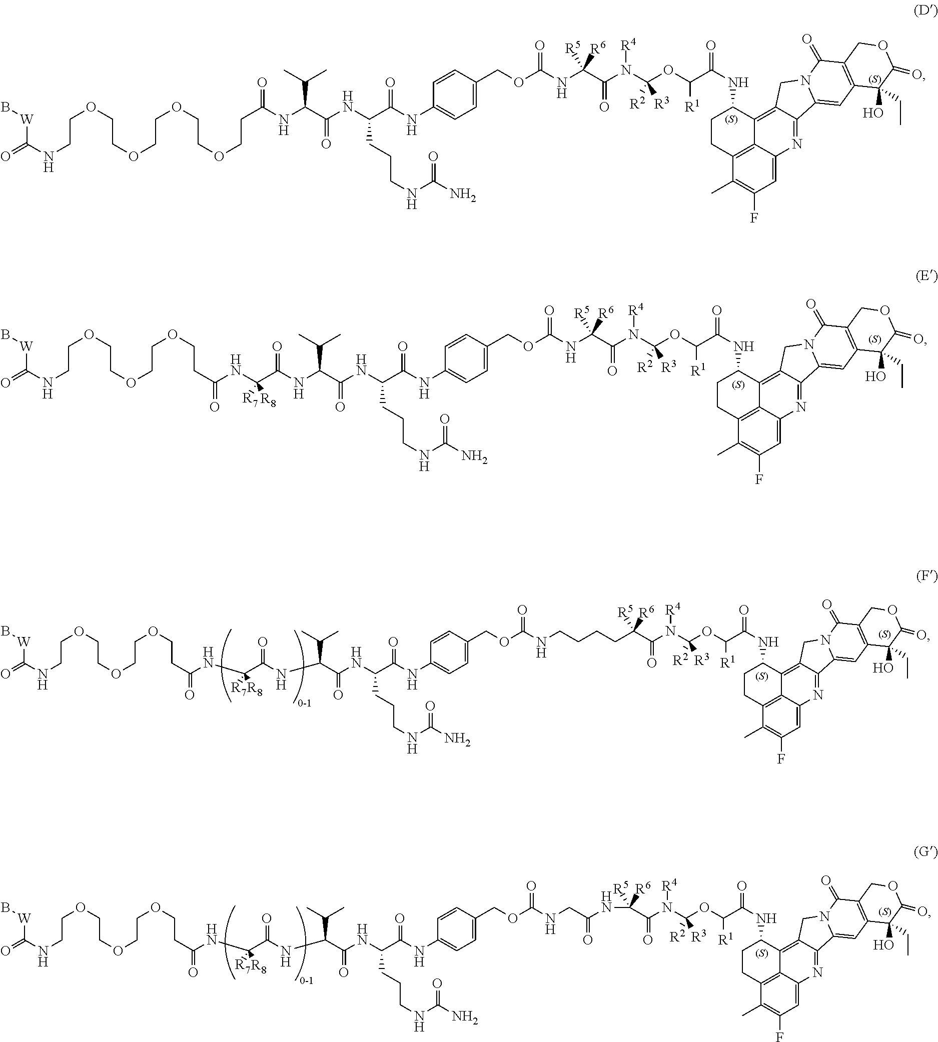 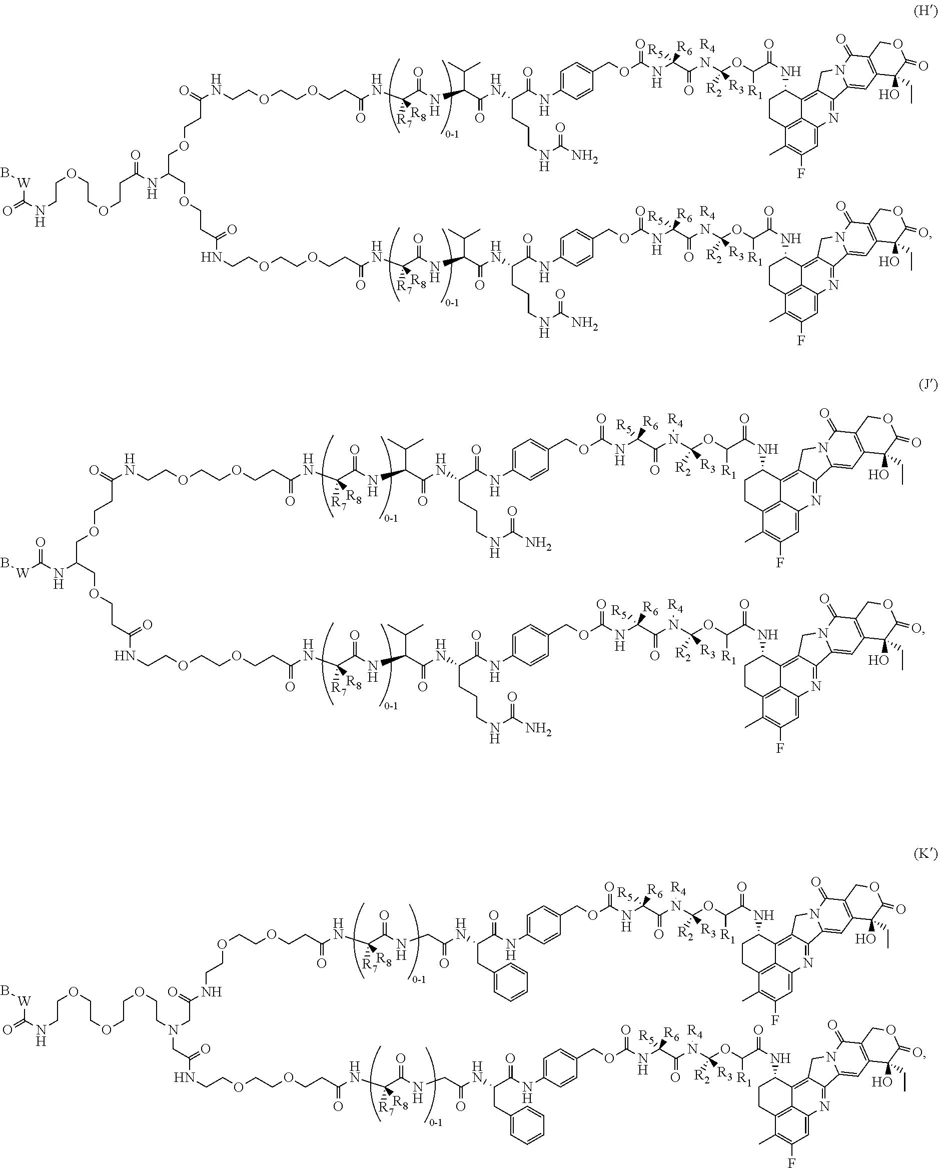 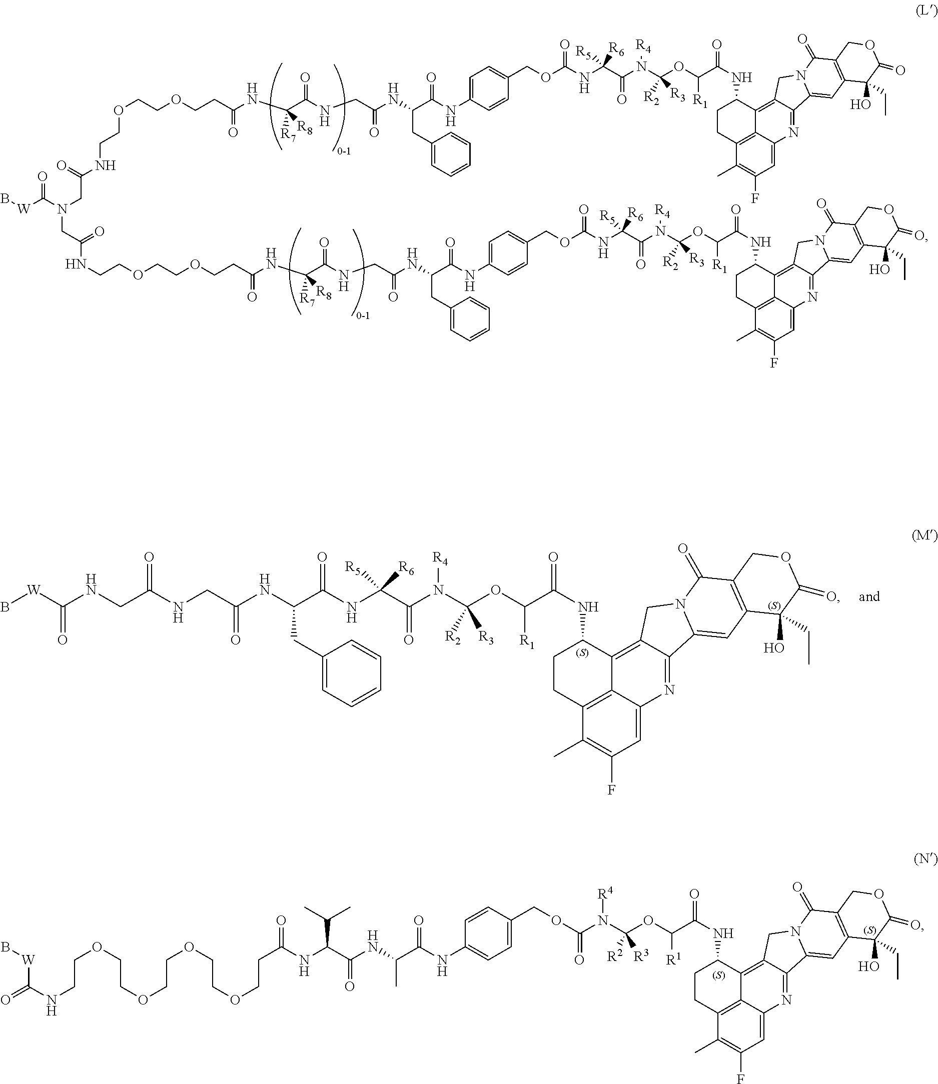
or a pharmaceutically acceptable salt thereof,
wherein R¹, R², R³, and R⁴ are independently hydrogen or a C₁₋₅ alkyl;
B is selected from the group consisting of 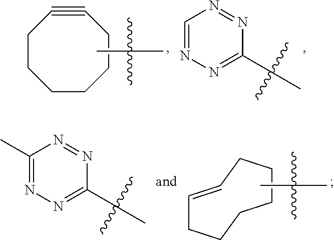
W is NH, O, CO, CH₂, a phenyl, or a combination of two or more thereof, and
R⁵, R⁶, R⁷ and R⁸ are independently hydrogen, —NH₂, or a side chain of any natural or non-natural amino acid,
the method comprising a step of exposing a payload having an amino group to an activated intermediate having a para-nitro-phenyl carbonate in the presence of a base and a coupling catalyst to afford said linker-payload compound (D′)-(N′), wherein said coupling catalyst is 4-Hydroxy-2-methylquinoline (MeHYQ).

118. The process of claim 117, wherein the linker-payload compound has the structure of formula (D-1) 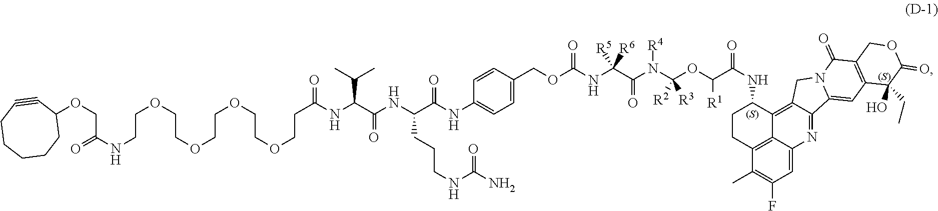
or a pharmaceutically acceptable salt thereof,
wherein R¹, R², R³, and R⁴ are independently hydrogen or a C₁₋₅ alkyl, and
R⁵ and R⁶ are independently hydrogen, —NH₂, or a side chain of any natural or non-natural amino acid,
the process comprising a step of exposing a drug payload having an amino group to an activated intermediate having a para-nitro-phenyl carbonate in the presence of a base and a coupling catalyst to afford said linker-payload compound (D-1), wherein said coupling catalyst is 4-Hydroxy-2-methylquinoline (MeHYQ).

119. The process of claim 117, wherein the linker-payload compound has the structure of Formula (D-1): 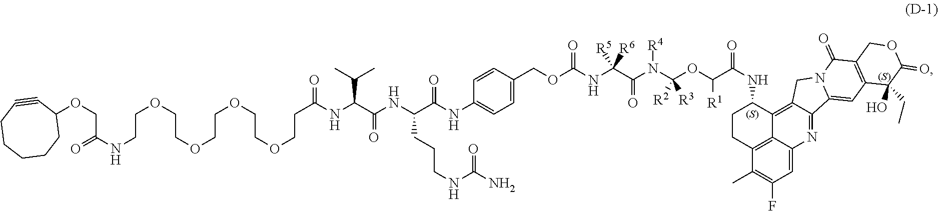
or a pharmaceutically acceptable salt thereof,
wherein R¹, R², R³, and R⁴ are independently hydrogen or a C₁₋₅ alkyl, and
R⁵ and R⁶ are independently hydrogen, —NH₂, or a side chain of any natural or non-natural amino acid,
said process comprising:
(a) providing a compound of Formula (I-1) having the structure: 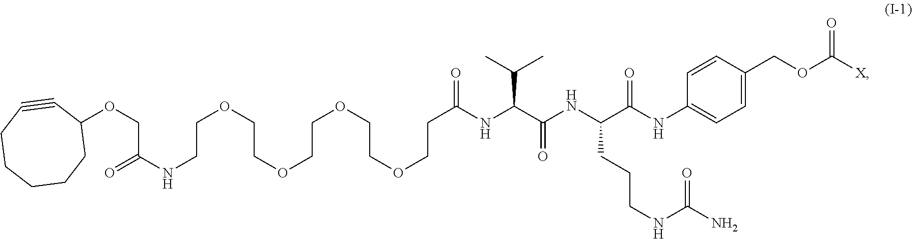
wherein
X is selected from the group consisting of 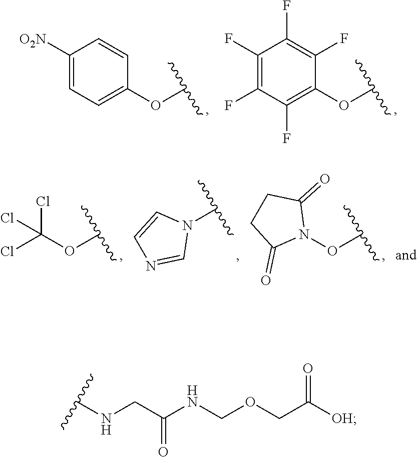
and
(b) reacting the compound of Formula (I-1) with a compound of Formula (P-I): 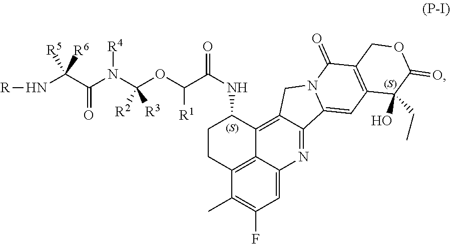
wherein
R is H or PG; and
PG is a suitable protecting group;
to produce the compound of Formula (D-1).

120. The process of claim 119, wherein the compound of Formula (D-1) has the following structure:

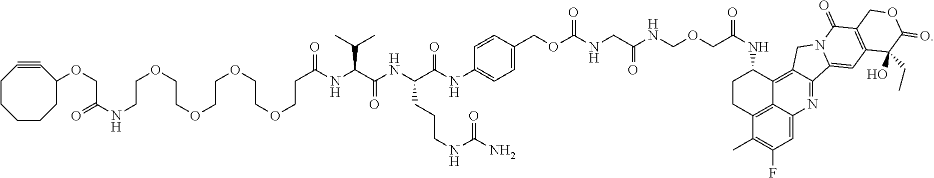 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

121. The process of claim 119, wherein the compound of Formula (I-1) has the following structure:

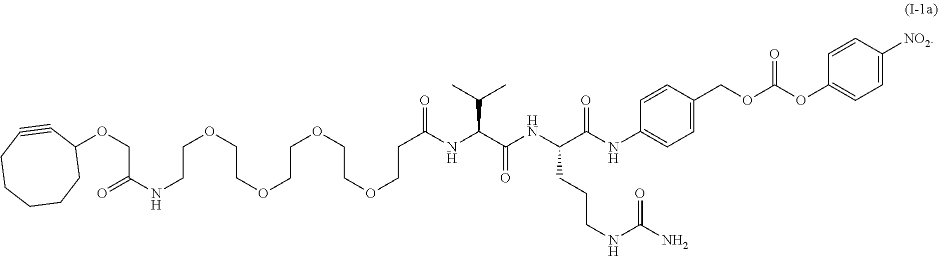 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(COC(=O)Oc2ccc([N+](=O)[O-])cc2)cc1]

122. The process of claim 119, wherein the compound of Formula (P-I) has the following structure:

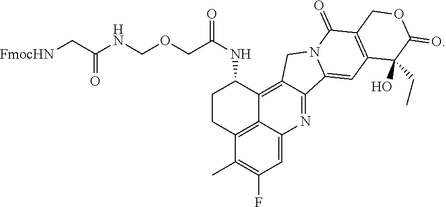

123. The process according to claim 119 further comprising the steps of:
providing a compound of Formula (V) having the structure: 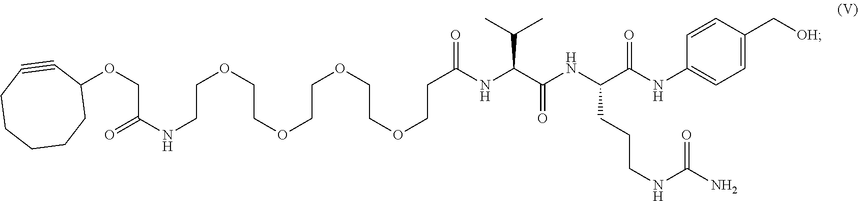 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(CO)cc1]
and
forming the compound of Formula (I-1) from the compound of Formula (V) prior to the step (a).

124. The process according to claim 119 wherein the compound of Formula (I-1) is prepared according to the steps of:
(a) providing a compound of Formula (V) having the structure: 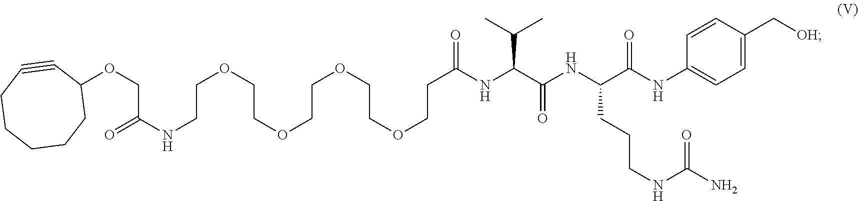 [SMILES: CC(C)[C@H](NC(=O)CCOCCOCCOCCOCCNC(=O)COC1C#CCCCCC1)C(=O)N[C@@H](CCCNC(N)=O)C(=O)Nc1ccc(CO)cc1]
and
(b) forming the compound of Formula (I-1) from the compound of Formula (V).

125. The process according to claim 124, wherein said step (b) of forming the compound of Formula (I-1) comprises:
reacting the compound of Formula (V) with a compound of Formula (VIa) or Formula (VIb): 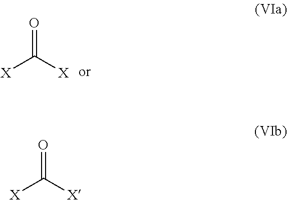
wherein
X′ is halogen,
to produce the compound of Formula (I-1).

126. The process according to claim 124 further comprising the steps of:
providing a compound of Formula (VII) having the structure: 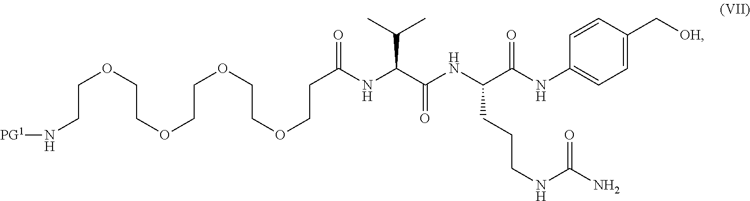
wherein PG¹ is a suitable protecting group protecting group, and
forming the compound of Formula (V) from the compound of Formula (VII).

127. The process of claim 126, wherein the compound of Formula (VII) has the following structure:

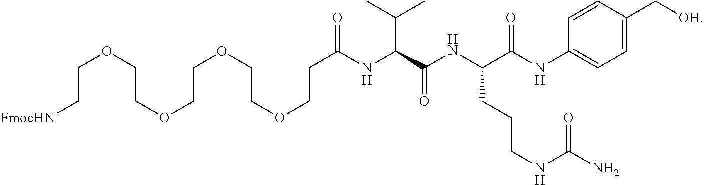

128. The process according to claim 126, wherein said step of forming the compound of Formula (V) comprises:
reacting the compound of Formula (VII) with a compound of Formula (VIII): 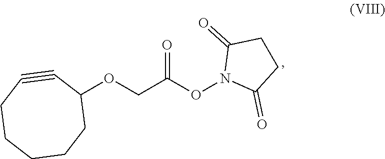 [SMILES: O=C(COC1C#CCCCCC1)ON1C(=O)CCC1=O]
to produce the compound of Formula (V).

129. The process according to claim 126 further comprising the steps of:
providing a compound of Formula (IX) having the structure: 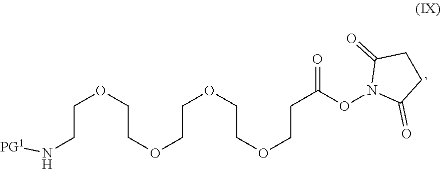
and
forming the compound of Formula (VII) from the compound of Formula (IX).

130. The process of claim 129, wherein the compound of Formula (IX) has the following structure:

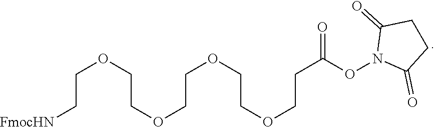

131. The process according to claim 129, wherein said step of forming the compound of Formula (VII) comprises:
reacting the compound of Formula (IX) with a compound of Formula (X): 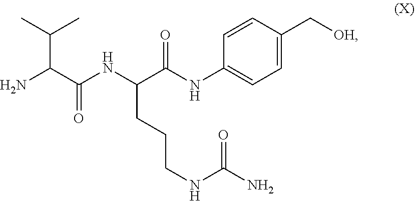 [SMILES: CC(C)C(N)C(=O)NC(CCCNC(N)=O)C(=O)Nc1ccc(CO)cc1]
to produce the compound of Formula (VII).

132. The process according to claim 129 further comprising the steps of:
providing a compound of Formula (XI) having the structure: 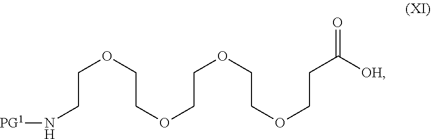
and
forming the compound of Formula (IX) from the compound of Formula (XI).

133. The process of claim 132, wherein the compound of Formula (XI) has the following structure:

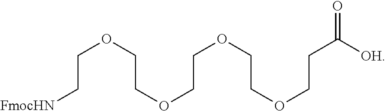

134. The process according to claim 132, wherein said step of forming the compound of Formula (IX) comprises:
reacting the compound of Formula (XI) with a compound of Formula (XII): 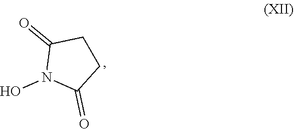
to produce the compound of Formula (IX).

135. The process of claim 119, wherein the compound of Formula (D-1) has the following structure:

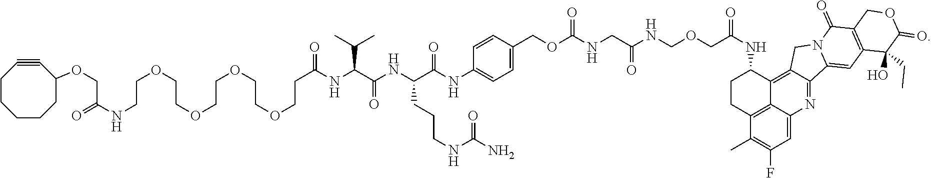 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

136. The method of claim 135, wherein said step (b) of reacting the compound of Formula (I-1) with the compound of Formula (P-I) further comprises:
reacting the compound of Formula (P-I), wherein R is PG, with a protecting group removing agent prior to said reacting with the compound of Formula (I-1).

137. The process according to claim 135, wherein the PG is selected from the group consisting of allyloxycarbonyl (Alloc), benzyloxycarbonyl (Cbz), tert-butyloxycarbonyl (Boc), and 9-fluorenylmethoxycarbonyl (Fmoc).

138. The process according to claim 135, wherein the protecting group removing agent is selected from the group consisting of Pd(PPh)₃, PhSiH₃, H₂, piperidine, and trifluoroacetic acid (TFA).

139. The process of claim 135, wherein the compound of Formula (P-I) has the following structure:

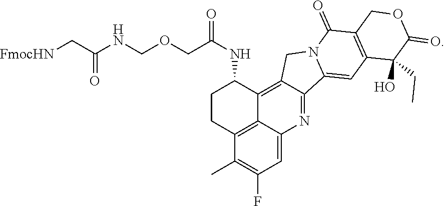

140. The process according to claim 135, further comprising the steps of:
providing a compound of Formula (XVIII) having the structure: 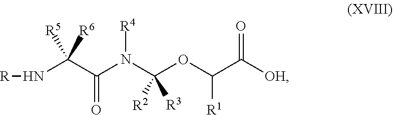
and
forming the compound of Formula (P-I) from the compound of Formula (XVIII).

141. The process of claim 140, wherein the compound of Formula (XVIII) has the following structure:

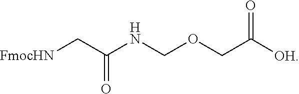

142. The process according to claim 140, wherein the step of forming the compound of Formula (P-I) comprises:
reacting the compound of Formula (XVIII) with a compound of Formula (XIX): 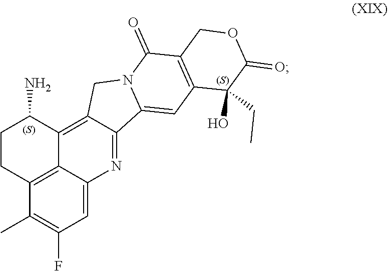
to produce the compound of Formula (P-I).

143. A process for preparation of a compound of Formula (D-1): 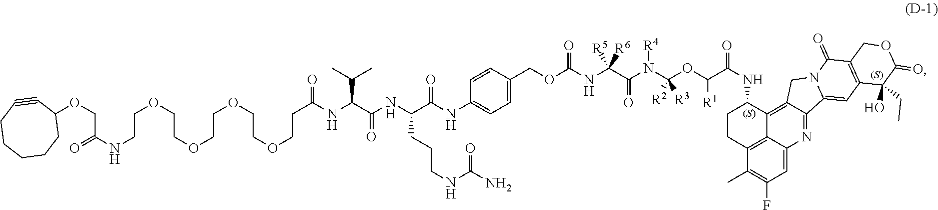
or a pharmaceutically acceptable salt thereof,
wherein R¹, R², R³, and R⁴ are independently hydrogen or a C₁₋₅ alkyl, and
R⁵ and R⁶ are independently hydrogen, —NH₂, or a side chain of any natural or non-natural amino acid,
said process comprising:
(a) providing a compound of Formula (XXIII): 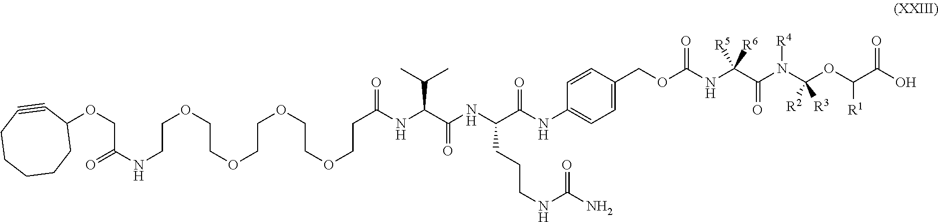
and
(b) reacting the compound of Formula (XXIII) with a compound having the structure: 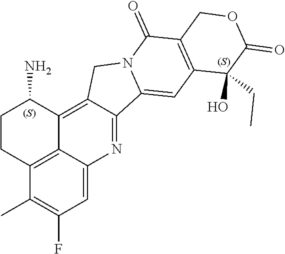
in the presence of an activating reagent and a base to produce the compound of Formula (D-1).

144. The process of claim 143, wherein the compound of Formula (D-1) has the following structure: 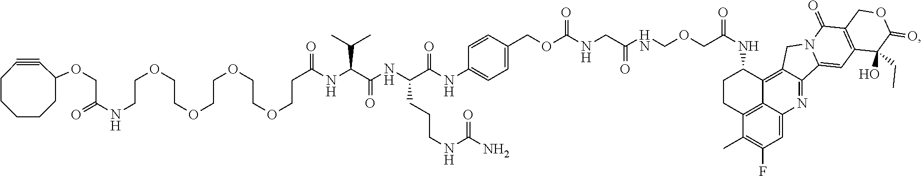 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]
